(19) United States
(12) Patent Application Publication
(10) Pub. No.: US 2024/0182445 A1
(43) Pub. Date: Jun. 6, 2024

(54) MODULATORS OF TREK (TWIK RELATED K+ CHANNELS) CHANNEL FUNCTION

(71) Applicants: ONO PHARMACEUTICAL CO., LTD., Osaka-shi, Osaka (JP); VANDERBILT UNIVERSITY, Nashville, TN (US)

(72) Inventors: Craig W LINDSLEY, Brentwood, TN (US); Darren W ENGERS, Brentwood, TN (US); Elizabeth S CHILDRESS, Franklin, TN (US); Sean R BOLLINGER, Chapel Hill, TN (US); Joza SCHMITT, Nashville, TN (US); Aaron T GARRISON, Franklin, TN (US); Charles K PERRY, Franklin, TN (US); Jerod S DENTON, Nashville, TN (US); Motoyuki TANAKA, Mishima-gun (JP)

(73) Assignees: ONO PHARMACEUTICAL CO., LTD., Osaka-shi, Osaka (JP); VANDERBILT UNIVERSITY, Nashville, TN (US)

(21) Appl. No.: 17/769,293

(22) PCT Filed: Oct. 23, 2020
(86) PCT No.: PCT/JP2020/040001

§ 371 Date: Apr. 14, 2022

Related U.S. Application Data
(63) Continuation-in-part of application No. PCT/CN2020/117116, filed on Sep. 23, 2020.
(60) Provisional application No. 62/925,470, filed on Oct. 24, 2019.

(30) Foreign Application Priority Data
Sep. 23, 2020 (WO) PCT/CN2020/117116

(51) Int. Cl. C07D 401/14; A61P 25/28; C07D 401/12; C07D 403/04; C07D 405/14; C07D 409/14; C07D 413/06; C07D 413/14; C07D 471/04

(52) U.S. Cl. CPC C07D 401/14; A61P 25/28; C07D 401/12; C07D 403/04; C07D 405/14; C07D 409/14; C07D 413/06; C07D 413/14; C07D 471/04

(57) ABSTRACT

Disclosed is a compound of formula (I): in which all symbols are defined in the description. Also disclosed are pharmaceutical compositions including the compounds, methods of making the compounds, kits comprising the compounds and methods of using the compounds to prevent and/or treat disorders associated with dysregulation of TREK-1, TREK-2 or both TREK-1 and TREK-2 in a mammal.

### TECHNICAL FIELD

The present disclosure relates to compounds, compositions, and methods for preventing and/or treating disorders associated with K2P K⁺ channels, specifically TREK (TWIK RElated K+ Channel) dysfunction for which modulators of TREK-1, TREK-2 or both TREK-1 and TREK-2 would provide therapeutic benefit.

### BACKGROUND

Potassium (K⁺) channels are membrane proteins that are expressed in virtually every cell of the organism. K⁺ channel subunits (˜80 genes) can be divided into three main structural classes comprising shaker type voltage-gated (Kv), inward rectifier (Kir) and K⁺ channels with two-pore domains (K2P) (Kubo et al., Pharmacol Rev. 2005, 57, 509, Gutman, et al. Pharmacol Rev. 2005, 57, 473, Goldstein et al. Pharmacol Rev. 2005, 57, 527). The third family of K⁺ channels was discovered 20 years ago (Leasge et al. EMBO J. 1996, 15, 1004). The 15 human K2P K⁺ channels have been identified so far and classified into 6 structural subgroups: TWIK, TREK (TWIK RElated K⁺ channels), TASK (TWIK related Acid-Sensitive K⁺ channels), TALK (TWIK related ALkaline pH-activated K⁺ channels), THIK (Tandem pore domain Halothane Inhibited K⁺ channels) and TRESK (TWIK RElated Spinal cord K⁺ channel) (Enyedi et al. Physiol. Rev. 2010, 90, 559). K2P K⁺ channels are responsible for background or ‘leak’ K⁺ currents. These channels are regulated by various physical and chemical stimuli, including membrane stretch, temperature, acidosis, lipids and inhalational anaesthetics. Furthermore, channel activity is tightly controlled by membrane receptor stimulation and second messenger phosphorylation pathways. Several members of this novel family of K⁺ channels are highly expressed in the central and peripheral nervous systems in which they are proposed to play an important physiological role (TRENDs in Neurosci. 2001).

TREK-1,TREK-2, which belong to TREK subgroup, are thermo- and mechano-gated K⁺ channel that is activated by lysophospholipids and PUFAs including arachidonic acid. They are regulated by G-protein-coupled receptors through PKA and PKC phosphorylation (Channels (Austin). 2011 September-October; 5(5):402-9). TREK-1 gene is widely expressed in the CNS with limited distribution in the periphery. In the CNS, TREK-1 expression is highest in the striatal tissues, the caudate and the putamen, as well as in spinal cord, foetal brain, amygdala and thalamus. In the periphery, TREK-1 expression is observed in heart, stomach and small intestine. TREK-2 gene has quite a similar expression profile compared to TREK-1 with high expression in particularly caudate, putamen and foetal brain. However, in contrast to TREK-1, TREK-2 is also highly expressed in cerebellum and corpus callosum well as in several peripheral tissues, particularly kidney (Mol. Brain Res. 2001, 86, 101).

TREK-1 deficient mice display an increased efficacy of serotonin (5-HT) neurotransmission, and a depression-resistant phenotype (Nature Neurosci. 2006, 9, 1134). Spadin, a naturally occurring peptide, blocks TREK-1 and results in a rapid onset of antidepressant efficacy (Br. J. Pharmacol. 2014, 172, 771). Moreover, anti-depressants such as fluoxetine and paroxetine directly inhibit TREK channels (Nat. Neurosci. 2006, 9, 1134; Br. J. Pharmacol. 2005, 144, 821). Thus, inhibition of TREK-1 with a small molecule holds promise for the treatment of depression, as well as other mood disorders (Front. Pharmacol. 2018, 9, 863).

Inhibition of TREK-1 protects mice from cognitive impairment induced by anesthesia and, coupled with a high density in the hippocampus, TREK-1 is a potential therapeutic target against memory impairment induced by volatile anesthetics and in other CNS disorders with cognitive deficits (Neurobiology of Learning and Memory, 2017, 145, 199). TREK-1 gene expression is increased in hippocampus of patients with schizophrenia compared to healthy control (Neuropsychopharmacology 2010, 35, 239-57.). Intrathecal injection of microRNA targeting to TREK-1 a meliorates neuropathic pain induced by chronic constriction sciatic nerve injury (Neurochem Res. 2018, 43, 1143), suggesting that inhibition of TREK-1 may be efficacious in cognitive disorders and neuropathic pain. Knockdown of TREK-1 significantly inhibits prostatic cancer cell proliferation in vitro and in vivo, and induces a G1/S cell cycle arrest (Cancer Res. 2008, 68, 1197-203, Oncotarget. 2015, 6, 18460-8.). TREK-1 is also overexpressed in human ovarian cancer tissues, and the treatment of TREK-1 inhibitors (curcumin and L-methionine) suppress ovarian cancer cell proliferation and increase late apoptosis (Clin. Transl. Oncol. 2013, 15, 910-8.). Thus, TREK-1 inhibitors can be useful for the treatment of prostatic and ovarian cancer.

Suppression of TREK-1 channels have been shown to be contributed to the potentiating action of AVP on CRH evoked ACTH secretion. Thus, an increase in the opening of the TREK-1 channel will oppose the stimulatory effect of CRH and AVP on the electrical excitability of corticotropes and will, in turn, reduce the stress-induced ACTH release, which leads to the belief that TREK-1 activators are also useful for the disease with abnormally high levels of cortisol, e.g., Cushing's syndrome (Endocrinology 2015, 156, 3661). TREK-1 activators are also useful for nasal inflammation (Sci Rep. 2015, 5, 9191), acute respiratory distress syndrome (acute lung injury) (Am. J. Physiol. Lung Cell Mol. Physiol. 2015, 308, L731), overactive bladder (J. Pharmacol. Exp. Ther. 2005, 313250), amyotrophic lateral sclerosis (Mol. Pharmacol. 2000, 57, 906), sepsis (J. Surg. Res. 2015, 193, 816), pancreatic cancer (Biochim. Biophys. Acta. 2016, 1862, 1994).

Neurotensin (NT) suppresses TREK-2 current through NT receptor 1-mediated activation of PLC/PKC pathway in entorhinal cortex layer II stellate neurons, leading to depolarization of membrane potential and enhancement of neuronal excitability. Furthermore, NT-induced enhancement of spatial learning is diminished in TREK-2 KO mice, suggesting that TREK-2 inhibitors may be useful for the treatment of cognitive impairment, such as Alzheimer's disease (J. Neurosci. 2014, 34, 7027-42.). TREK-2 is expressed in human bladder carcinoma cell in which TREK-2 contributes to the regulation of resting membrane potential. TREK-2 KD decreases the cell proliferation (Korean J. Physiol. Pharmacol. 2013, 17, 511-6.). Thus, TREK-2 inhibitors also may be efficacious in the treatment of bladder carcinoma.

TREK-2 channels are expressed in the kidney, proximal convoluted tubule epithelial cells, and that polycystins protect renal epithelial cells against apoptosis in response to mechanical stress, and this function is mediated through the opening of TREK-2 (Cell Rep. 2012, 1, 241). Thus, TREK-2 activators are useful for autosomal dominant polycystic kidney disease. TREK-2 channels are functionally upregulated in astrocytes after ischemia and rescue astrocytic buffering of glutamate, which leads to the belief that TREK-2 activators are also useful for ischemia (Open Neurosci J. 2009, 3, 40). The entorhinal cortex is closely associated with the consolidation and recall of memories, Alzheimer disease, schizophrenia, and temporal lobe epilepsy. Norepinephrine is a neurotransmitter that plays a significant role in these physiological functions and neurological diseases. Norepinephrine activates TREK-2 via alpha 2A adrenergic receptors-mediated inhibition of the protein kinase A pathway, which leads to hyperpolarizes membrane potential and depresses neuronal excitability (J. Biol Chem. 2009, 284, 10980, ACS Chem Neurosci. 2016 Sep. 22 (WEB ASAP)).

Selective inhibition of TREK-1, by a small inhibitor, has potential therapeutic benefit for: depression, schizophrenia, cognitive disorders including dementia, neuropathic pain, stroke, prostatic and ovarian cancer (Nat. Neurosci. 2006, 9, 1134, Neuropsychopharmacology 2010, 35, 239, Neurobiol. Learn Mem. 2017, 145, 199, Neurochem Res. 2018, 43, 1143, Neurosci Lett. 2018, 671, 93, Cancer Res. 2008, 68, 1197, Clin. Transl. Oncol. 2013, 15, 910).

Selective activation of TREK-1, by a small molecule activator, has potential therapeutic benefit for: pain, nasal inflammation, atrial fibrillation, acute respiratory distress syndrome, cerebreal ischemia, overactive bladder, epilepsy, amyotrophic lateral sclerosis, anaesthesia, neuronal degeneration diseases, sepsis, pancreatic cancer and Cushing's syndrome (Nat. Commun. 2013, 4, 2941, Sci. Rep. 2015, 5, 9191, Life Sci. 2014, 97, 107, EMBO J. 2004, 23, 2684, Mol. Pharmacol. 2000, 57, 906, Biochim. Biophys. Acta. 2016, 1862, 1994, Endocrinology 2015, 156, 3661).

Selective inhibition of TREK-2, by a small molecule inhibitor, has potential therapeutic benefit for: cognitive disorders including dementia, stroke and bladder carcinoma (J. Neurosci. 2014, 34, 7027, Biochem. Biophys. Res. Commun. 2005, 327, 1163, Korean J. Physiol. Pharmacol. 2013, 17, 511). The expression level of TREK-2 is increased in cortex and hippocampus of acute rat cerebral ischemia model (Biochem. Biophys. Res. Commun. 2005, 327, 1163-9.) Thus, TREK-2 inhibitors may be useful for the treatment of stroke.

Selective activation of TREK-2, by a small molecule activator, has potential therapeutic benefit for: pain, ischemia, autosomal dominant polycystic kidney disease, osteoporosis, anaesthesia, temporal lobe epilepsy and schizophrenia (J. Neurosci. 2014, 34, 1494, Open Neurosci J. 2009, 3, 40, Cell Rep. 2012, 1, 41, J. Bone Miner. Res. 2005, 20, 1454, Neurosci. Lett. 2016, 619, 54, J. Biol. Chem. 2009, 284, 10980, ACS Chem Neurosci. 2016 WEB ASAP).

All of the above mentioned disorders may also be effectively treated by a dual TREK1/TREK2 inhibitor or activator with varying degrees of TREK1 and TREK2 preference.

Despite advances in K2P channel research and TREK1/TREK2 pharmacology channel research, there is still a scarcity of compounds that are potent, efficacious, and selective inhibitors of the either TREK1, TREK2 or both TREK1 and TREK2 and also effective in the treatment of neurological, inflammatory, respiratory, renal and cardiovascular disorders associated with K2P K⁺ channels, specifically TREK (TWIK RElated K⁺ channels) dysfunction for which inhibitors of TREK1, TREK2 or both TREK1 and TREK2 would offer therapeutic benefit, or a scarcity of compounds that are potent, efficacious, and selective activators of the either TREK1, TREK2 or both TREK1 and TREK2 and also effective in the treatment of disorders associated with K2P K⁺ channels, specifically TREK (TWIK RElated K⁺ channels) dysfunction for which activators of TREK1, TREK2 or both TREK1 and TREK2 would offer therapeutic benefit.

PTL 1 discloses that the compound represented of formula (A):

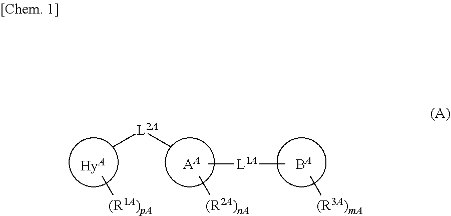

or a pharmaceutically acceptable salt thereof, wherein;
Hy^{A} is 5-6 membered heteroaryl ring having 1-2 nitrogens or the like;
Ring A^{A} is phenyl, pyridyl or the like;
L^{2A} is

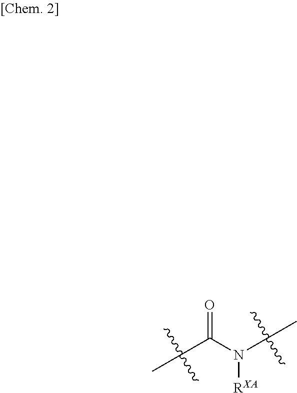

or the like;
each R^{XA} is independently hydrogen or the like;
Ring B^{A} is phenyl, 5-6 membered monocyclic heteroaromatic ring having 1-4 heteroatoms independently selected from nitrogen, oxygen, or sulfur, or the like;
each R^{1A} is independently selected from RA or the like;
each R^{A} is independently hydrogen, an optionally substituted group selected from C1-6 aliphatic, a 3-8 membered saturated or partially unsaturated monocyclic carbocyclic ring or the like;
L^{1A} is an optionally substituted C1-4 membered straight or branched saturated or unsaturated bivalent hydrocarbon chain or the like;
each of R^{2A} and R^{3A} is independently selected from R^{A}, halogen, cyano, OR^{A} or the like;
each of m and n is independently 1, 2, 3, 4, or 5; and p is 0, 1, 2, or 3;
is an inhibitor of the aryl hydrocarbon receptor and is useful for treating cancer.

### CITATION LIST

### Patent Literature

PTL 1: WO2019/036657

### SUMMARY

In one aspect, disclosed are compounds of formula (I),

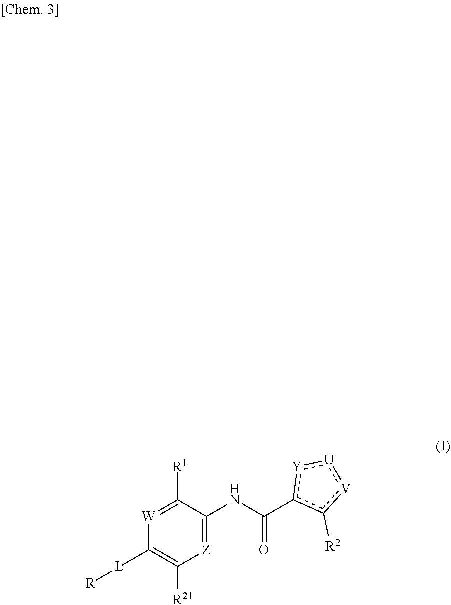

or a pharmaceutically acceptable salt thereof;
wherein: all symbols are defined as below.

Also disclosed are pharmaceutical compositions comprising the compounds, methods of making the compounds, kits comprising the compounds, and methods of using the compounds, compositions and kits for prevention and/or treatment of disorders, such as neurological and/or psychiatric disorders, associated with TREK1, TREK2 or both TREK1/TREK2 dysfunction which inhibitors of TREK1, TREK2 or both TREK1 and TREK2 would offer therapeutic benefit in a mammal, or disorders associated with TREK1, TREK2 or both TREK1/TREK2 dysfunction for which activators of TREK1, TREK2 or both TREK1 and TREK2 would offer therapeutic benefit in mammal.

### BRIEF DESCRIPTION OF DRAWINGS

FIG. 1; Shows that the Compound 12 caused reversal of MK-801 induced deficits in the Novel Object Recognition Task. The vertical axis shows the Recognition Index, and the horizontal axis shows the group to which vehicle, vehicle+MK-801 or the test compound was administered. (*p<0.05 compared to vehicle+MK-801-treated group (Dunnett's test), #p<0.07 compared to vehicle+MK-801-treated group (Dunnett's test))

### DESCRIPTION OF EMBODIMENTS

Disclosed herein are modulators of TREK1, TREK2 or both TREK1/TREK2.

That is, the invention relates to;

[1] A compound of formula (I):

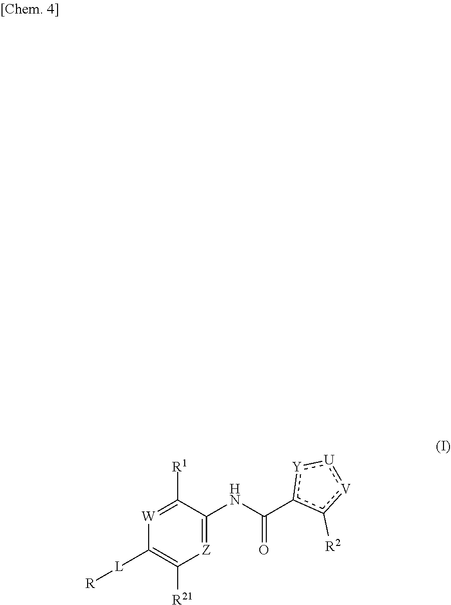

or a pharmaceutically acceptable salt thereof;
wherein:
L is selected from (1) bond, (2) C2-C4-alkynylene, (3) C2-C4-alkenylene, (4) —(C1-C10-alkylene)-O—, (5) —O—(C1-C10-alkylene)-, (6) -(6 to 15 membered aryl)-, (7)-(5 to 15 membered heteroaryl)-, (8) -(3 to 15 membered heterocycle)- and (9) —(C3-C10-cycloalkane)-
W is selected from (1) CH, (2) CR⁴ and (3) N;
Z is selected from (1) CH, (2) CR⁵ and (3) N;
R⁴ and R⁸ is each independently selected from (1) cyano, (2) halogen, (3) pentahalosulfanyl, (4) C1-C10-thioalkyl, (5) C1-C10-alkoxy, (6) C1-C10-alkyl, (7) C2-C10-alkenyl, (8) C2-C10-alkynyl, (9) —OR^{b}, (10) 6 to 15 membered aryl, (11) 5 to 15 membered heteroaryl, (12) C3-C10-cycloalkyl, (13) C2-C10-heteroalkyl, (14) 3 to 15 membered heterocycle, and (15) —(CR^{c}R^{d})ₙ-Q,
wherein each of (4)-(8) in R⁴ or R⁸ may be optionally substituted with 1 to 10 halogen, and each of (10)-(14) in R⁴ or R⁸ may be optionally substituted with 1 to 10 substituents selected from (1) halogen, (2) C1-C10-alkyl, and (3) C1-C10-haloalkyl;
R^{b} is selected from (1) hydrogen, (2) C1-C10-alkyl, and (3) C1-C10-haloalkyl;
R^{c} is selected from (1) hydrogen, (2) halogen, (3) C1-C10-alkyl and (4) C1-C10-haloalkyl;
R^{d} is selected from (1) hydrogen, (2) halogen, (3) C1-C10-alkyl and (4) C1-C10-haloalkyl;
or when R^{c} and R^{d} are C1-C10-alkyl, R^{c} and R^{d} may optionally form a C3-C10-cycloalkyl together with the carbon atom to which they are attached;
n is 0, 1, 2, 3 or 4;
Q is selected from (1) halogen, (2) cyano, (3) —OR²⁰¹, (4) —SR²⁰², (5) —C(═O)R²⁰³, (6) —C(═O)OR²⁰⁴, (7) —S(═O)R²⁰⁵, (8) —SO₂R²⁰⁶, (9) —N(R²⁰⁷)₂, (10) —C(═O)N(R²⁰⁸)₂, (11) —SO₂N(R²⁰⁹)₂, (12) 6 to 15 membered aryl, (13) 5 to 15 membered heteroaryl, (14) C3-C10-cycloalkyl, and (15) 3 to 15 membered heterocycle, wherein each of (12) -(15) in Q may be optionally substituted with 1 to 10 substituents selected from (1) halogen, (2) C1-C10-alkyl and (3) C1-C10-haloalkyl;
R²⁰¹, R²⁰², R²⁰³, R²⁰⁴, R²⁰⁵, R²⁰⁶, R²⁰⁷, R²⁰⁸ and R²⁰⁹ are each independently selected from (1)hydrogen, (2) C1-C10-alkyl, and (3) C1-C10-haloalkyl, and multiple R²⁰⁷, R²⁰⁸ and R²⁰⁹ may be the same as or different from each other;

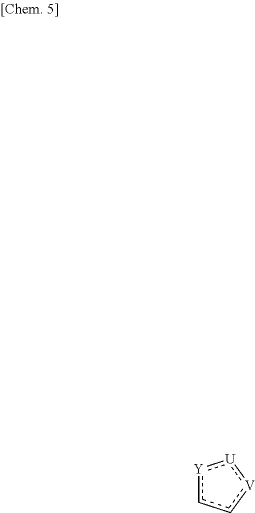

is selected from (1) 1,2,3-triazole substituted with R³, (2) pyrrole substituted with R³, (3) pyrazole substituted with R³, (4) isoxazole substituted with R³, (5) isothiazole substituted with R³, (6) imidazole substituted with R³, (7) furan substituted with R³, (8) thiophene substituted with R³;
wherein Y is selected from (1) CH, (2) CR³, (3) N, (4) NH, (5) NR³, (6) O and (7) S, U is selected from (1) CH, (2) CR³, (3) CR^{x}, (4) N, (5) NH, (6) NR³, (7) NR^{x}, (8) O and (9) S,
V is independently selected from (1) CH, (2) CR^{x}, (3) N, (4) NH, (5) NR^{x}, (6) O and (7) S, one of Y and U is NR³ or CR³, and the other is not NR³ or CR³,
[Chem. 6] [symbol]
is single bond or double bond,
R^{x} is selected from (1) NH₂, (2)halogen, (3) C1-C4-alkyl and (4) C1-C4-haloalkyl;
R is selected from (1)₆ to 15 membered aryl and (2)₅ to 15 membered heteroaryl, each of which may be optionally substituted with 1 to 5 R⁶;
wherein multiple R⁶ may be the same as or different from each other;
R⁶ is selected from (1) halogen, (2) cyano, (3) pentahalosulfanyl, (4) C1-C10-alkyl, (5) C1-C10-thioalkyl, (6) C1-C10-alkoxy, (7) C2-C10-alkenyl, (8) C2-C10-alkynyl and (9) —OR^{b}, wherein each of (4)-(6) in R⁶ is may be optionally substituted with 1 to 10 halogen;
R¹ is selected from (1) C1-C10-alkyl, (2) halogen, (3) C1-C10-alkoxy, (4) C1-C10-haloalkyl, (5) C1-C10-haloalkoxy and (6) cyano;
R²¹ is selected from (1) hydrogen, (2) C1-C10-alkyl, (3) halogen, (4) C1-C10-alkoxy, (5) C1-C10-haloalkyl, (6) C1-C10-haloalkoxy and (7) cyano;
R² is selected from (1) halogen, (2) C1-C10-alkyl, (3) C1-C10-haloalkyl, (4) cyano and (5) C3-C10-cycloalkyl which may be optionally substituted with 1 to 10 substituents selected from (1) halogen (2) C1-C10-alkyl and (3) C1-C10-haloalkyl;
R³ is selected from (1) R⁷ or

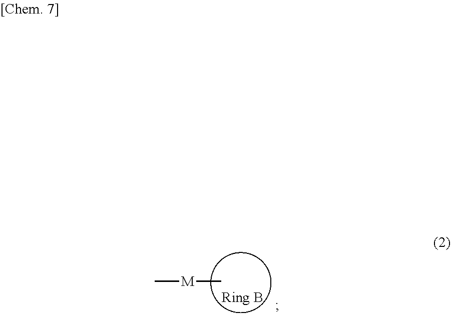

wherein R⁷ is selected from (1) C3-C10-alkyl, (2) -(C2-C10-alkylene)-cyano, (3) -(C1-C10-alkylene)-NH—C(═O)—O—(C1-C10-alkyl), (4) -(C1-C10-alkylene)-O—(C1-C10-alkyl) and (5) -(C1-C10-alkylene)-(CR⁷¹R⁷²)ₚ—R⁷³ each of (1)-(5) in R⁷ is may be optionally substituted with 1 to 10 halogen;
wherein R⁷¹ is selected from (1)hydrogen, (2)halogen, (3) C1-C10-alkyl and (4) C1-C10-haloalkyl;
R⁷² is selected from (1)hydrogen, (2)halogen, (3) C1-C10-alkyl and (4) C1-C10-haloalkyl;
or when R⁷¹ and R⁷² are C1-C10-alkyl, R⁷¹ and R⁷² may optionally form a C3-C10-cycloalkyl together with the carbon atom to which they are attached, wherein C3-C10-cycloalkyl may be optionally substituted with 1-10 substituents selected from (1) halogen and (2) cyano;
p is 0, 1, 2, 3 or 4;
R⁷³ is selected from (1) —OR¹⁰¹, (2) —SR¹⁰², (3) —C(═O)R¹⁰³, (4) —C(═O)OR¹⁰⁴, (5) —S(═O)R¹⁰⁵, (6) —SO₂R¹⁰⁶, (7) —N(R¹⁰⁷)₂, (8) —C(═O)N(R^{10′})₂, (9) —SO₂N(R¹⁰⁹)₂, (10)₆ to 15 membered aryl, (11)₅ to 15 membered heteroaryl, (12) C3-C10-cycloalkyl, and (13)₃ to 15 membered heterocycle, wherein each of (10)-(13) in R⁷³ may be optionally substituted with 1-10 substituents selected from (1) halogen, (2) C1-C10-alkyl and (3) C1-C10-haloalkyl;
R¹⁰¹, R¹⁰², R¹⁰³, R¹⁰⁴, R¹⁰⁵, R¹⁰⁶, R¹⁰⁷, R¹⁰⁸ and R¹⁰⁹ are each independently selected from (1)hydrogen, (2) C1-C10-alkyl, and (3) C1-C10-haloalkyl, and multiple R¹⁰⁷, R¹⁰⁸ and R¹⁰⁹ may be the same as or different from each other;
M is selected from (1) bond or (2) C1-C10-alkylene which may be optionally substituted with 1 to 3 halogens;
Ring B is selected from (1) C3-C10-cycloalkyl which may be optionally substituted with 1 to 5 R¹, (2) 3 to 15 membered heterocycle which may be optionally substituted with 1 to 5 R⁹, (3) 6 to 15 membered aryl which may be optionally substituted with 1 to 5 R¹⁰ and (4) 5 to 15 membered heteroaryl which may be optionally substituted with 1 to 5 R¹¹;
wherein multiple R⁸, R⁹, R¹⁰ or R¹¹ may be the same as or different from each other;
R⁸, R⁹, R¹⁰ and R¹¹ are each independently selected from (1) halogen, (2) C1-C10-alkyl, (3) cyano, (4) —C(═O)—(C1-C10-alkyl), (5) —C(═O)—(C3-C10-cycloalkyl), (6) —C(═O)-(3 to 15 membered heterocycle), (7) —C(═O)—O—(C1-C10-alkyl), (8) —C(═O)—O—(C3-C10-cycloalkyl), (9) —C(═O)—O-(3 to 15 membered heterocycle), (10) —C(═O)—NR⁸¹—(C1-C10-alkyl), (11) —C(═O)—NR⁸²—(C3-C10-cycloalkyl), (12) —C(═O)—NR⁸³-(3 to 15 membered heterocycle), (13) —NR⁸⁴—C(═O)—(C1-C10-alkyl), (14) —NR⁸⁵C(═O)—(C3-C10-cycloalkyl), (15) —NR⁸⁶—C(═O)—(3 to 15 membered heterocycle), (16) —NR⁸⁷—C(═O)—O—(C1-C10-alkyl), (17) —NR^{II}—C(═O)—O—(C3-C10-cycloalkyl), (18) —NR⁸⁹—C(═O)—O-(3 to 15 membered heterocycle), (19) —SO₂—(C1-C10-alkyl) and (20) oxo;
wherein each of (2), (4), (7), (10), (13), (16) and (19) in R⁸, R⁹, R¹⁰ or R¹¹ may be optionally substituted with 1 to 10 substituents selected from (1) halogen, (2) —OH and (3) C1-C10-alkoxy, and each of (5), (6), (8), (9), (11), (12), (14), (15), (17) and (18) in R⁸, R⁹, R¹⁰ or R¹¹ may be optionally substituted with 1 to 10 substituents selected from (1) halogen, (2) —OH, (3) C1-C10-alkyl, (4) C1-C10-alkoxy and (5) C1-C10-haloalkyl;
R⁸¹, R⁸², R⁸³, R⁸⁴, R⁸⁵, R⁸⁶, R⁸⁷, R⁸⁸ and R⁸⁹ are independently selected from (1)hydrogen and (2) C1-C4-alkyl.

[2] The compound or a pharmaceutically acceptable salt thereof according to [1], which is a compound of formula (Ia):

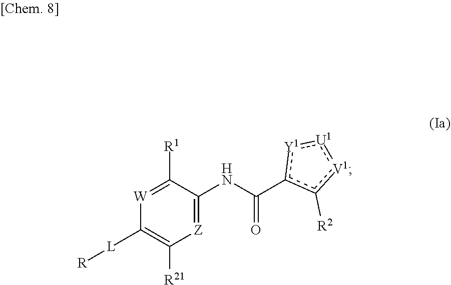

wherein

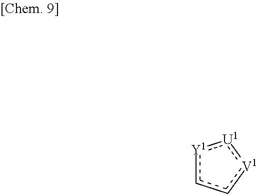

is selected from (1) 1,2,3-triazole substituted with R³, (2) pyrrole substituted with R³, (3) pyrazole substituted with R³ and (4) imidazole substituted with R³; wherein Y¹ is independently selected from CR³ or NR³;
U¹ is selected from (1) CH, (2) NR^{x} and (3) N;
V¹ is selected from (1) CH and (2) N;
the other symbols are as defined in [1].

[3] The compound or a pharmaceutically acceptable salt thereof according to [2], which is a compound of formula (Ia-1):

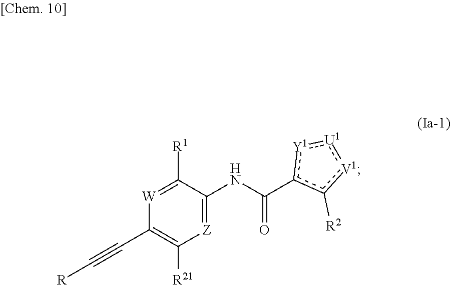

wherein all symbols are as defined in [1] or [2].

[4-1] The compound or a pharmaceutically acceptable salt thereof according to [3], wherein R¹ is C1-C4-alkyl, halogen, C1-C4-alkoxy, C1-C4-haloalkyl or C1-C4-haloalkoxy (preferably C1-C4-alkyl, C1-C4-haloalkyl or halogen, more preferably C1-C4-alkyl or halogen, most preferably methyl or halogen);
R² is halogen, C1-C4-alkyl or C1-C4-haloalkyl (preferably C1-C4-alkyl or halogen, more preferably halogen);
R²¹ is hydrogen, C1-C4-alkyl, halogen, C1-C4-alkoxy, C1-C4-haloalkyl or C1-C4-haloalkoxy (preferably hydrogen);

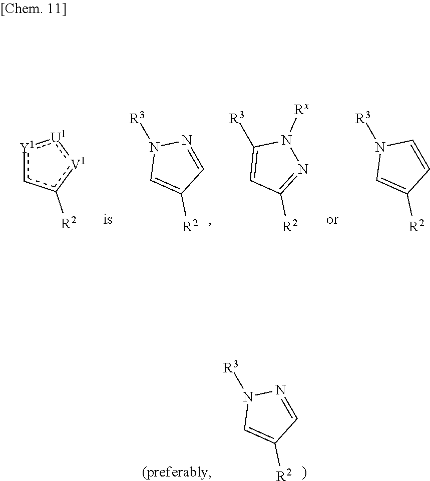

R^{x} is NH₂, halogen or methyl (preferably NH₂ or methyl);
R³ is —(C2-C6-alkylene)-cyano which may be optionally substituted with 1 to 10 halogen, —(C1-C6-alkylene)-NH—C(═O)—O—(C1-C6-alkyl) which may be optionally substituted with 1 to 10 halogen, —(C1-C6-alkylene)-O—(C1-C6-alkyl) which may be optionally substituted with 1 to 10 halogen, C3-C8-cycloalkyl which may be optionally substituted with 1 to 5 R¹, 3 to 10 membered heterocycle which may be optionally substituted with 1 to 5 R⁹, 6 to 10 membered aryl which may be optionally substituted with 1 to 5 R¹⁰, 5 to 10 membered heteroaryl which may be optionally substituted with 1 to 5 R¹¹, —(C1-C4-alkylene)—(C3-C8-cycloalkyl which may be optionally substituted with 1 to 5 R¹), —(C1-C4-alkylene)-(3 to 10 membered heterocycle which may be optionally substituted with 1 to 5 R⁹), —(C1-C4-alkylene)-(6 to 10 membered aryl which may be optionally substituted with 1 to 5 R¹⁰) or —(C1-C4-alkylene)-(5 to 10 membered heteroaryl which may be optionally substituted with 1 to 5 R¹⁰) (preferably —(C2-C4-alkylene)-cyano which may be optionally substituted with 1 to 5 halogen, —(C3-C6-alkylene)-NH—C(═O)—O—(C3-C6-alkyl) which may be optionally substituted with 1 to 10 halogen, —(C3-C6-alkylene)-O—(C3-C6-alkyl) which may be optionally substituted with 1 to 10 halogen, C3-C8-cycloalkyl which may be optionally substituted with 1 to 5 R¹, 3 to 10 membered heterocycle which may be optionally substituted with 1 to 5 R⁹, —(C1-C4-alkylene)—(C3-C8-cycloalkyl which may be optionally substituted with 1 to 5 R¹), or —(C1-C4-alkylene)-(3 to 10 membered heterocycle which may be optionally substituted with 1 to 5 R⁹), more preferably —(C2-C4-alkylene)-cyano which may be optionally substituted with 1 to 5 halogen, —(C3-C6-alkylene)-NH—C(═O)—O—(C3-C6-alkyl) which may be optionally substituted with 1 to 10 halogen, —(C3-C6-alkylene)-O—(C3-C6-alkyl) which may be optionally substituted with 1 to 10 halogen, C3-C8-cycloalkyl which may be optionally substituted with 1 to 5 R¹, 3 to 10 membered heterocycle which may be optionally substituted with 1 to 5 R⁹, —(C1-C2-alkylene)—(C3-C8-cycloalkyl which may be optionally substituted with 1 to 5 R¹), or —(C1-C2-alkylene)-(3 to 10 membered heterocycle which may be optionally substituted with 1 to 5 R⁹), most preferably —(C2-C4-alkylene)-cyano which may be optionally substituted with 1 to 5 halogen (preferably —(C2-C4-alkylene)-cyano), —(C3-C6-alkylene)-NH—C(═O)—O—(C3-C6-alkyl) which may be optionally substituted with 1 to 10 halogen, —(C3-C6-alkylene)-O—(C3-C6-alkyl) which may be optionally substituted with 1 to 10 halogen (preferably —(C3-C6-alkylene)-O—(C3-C6-alkyl)), C3-C8-cycloalkyl which may be optionally substituted with 1 to 5 R⁸, 4 to 7 membered heterocycle which may be optionally substituted with 1 to 5 R⁹, —(C1-C2-alkylene)—(C3-C8-cycloalkyl which may be optionally substituted with 1 to 5 R⁸), or —(C1-C2-alkylene)-(4 to 7 membered heterocycle which may be optionally substituted with 1 to 5 R⁹);
R⁸ is halogen, cyano, C1-C4-alkyl which may be optionally substituted with 1 to 5 substituents selected from (1) halogen, (2) —OH and (3) C1-C4-alkoxy, —C(═O)—(C1-C4-alkyl) which may be optionally substituted with 1 to 5 substituents selected from (1) halogen, (2) —OH and (3) C1-C4-alkoxy, —C(═O)—(C3-C7-cycloalkyl which may be optionally substituted with 1 to 5 substituents selected from (1) halogen, (2) —OH, (3) C1-C4-alkyl, (4) C1-C4-alkoxy and (5) C1-C4-haloalkyl), —C(═O)—(3 to 10 membered heterocycle which may be optionally substituted with 1 to 5 substituents selected from (1) halogen, (2) —OH, (3) C1-C4-alkyl, (4) C1-C4-alkoxy and (5) C1-C4-haloalkyl), —C(═O)—O—(C1-C4-alkyl) which may be optionally substituted with 1 to 5 substituents selected from (1) halogen, (2) —OH and (3) C1-C4-alkoxy, —C(═O)—NH—(C3-C7-cycloalkyl which may be optionally substituted with 1 to 5 substituents selected from (1) halogen, (2) —OH, (3) C1-C4-alkyl, (4) C1-C4-alkoxy and (5) C1-C4-haloalkyl), —NH—C(═O)—(C1-C4-alkyl) which may be optionally substituted with 1 to 5 substituents selected from (1) halogen, (2) —OH and (3) C1-C4-alkoxy, —NH—C(═O)—(C3-C7-cycloalkyl which may be optionally substituted with 1 to 5 substituents selected from (1) halogen, (2) —OH, (3) C1-C4-alkyl, (4) C1-C4-alkoxy and (5) C1-C4-haloalkyl), —NH—C(═O)—(3 to 10 membered heterocycle which may be optionally substituted with 1 to 5 substituents selected from (1) halogen, (2) —OH, (3) C1-C4-alkyl, (4) C1-C4-alkoxy and (5) C1-C4-haloalkyl), —NH—C(═O)—O—(C1-C4-alkyl) which may be optionally substituted with 1 to 5 substituents selected from (1) halogen, (2) —OH and (3) C1-C4-alkoxy, or —SO₂—(C1-C4-alkyl) which may be optionally substituted with 1 to 5 substituents selected from (1) halogen, (2) —OH and (3) C1-C4-alkoxy (preferably halogen, cyano, C1-C4-alkyl which may be optionally substituted with 1 to 5 substituents selected from (1) halogen, (2) —OH and (3) C1-C4-alkoxy, —NH—C(═O)—(C1-C4-alkyl) which may be optionally substituted with 1 to 5 substituents selected from (1) halogen, (2) —OH and (3) C1-C4-alkoxy, —NH—C(═O)—(C3-C7-cycloalkyl which may be optionally substituted with 1 to 5 substituents selected from (1) halogen, (2) —OH, (3) C1-C4-alkyl, (4) C1-C4-alkoxy and (5) C1-C4-haloalkyl), —NH—C(═O)—(4 to 6 membered heterocycle which may be optionally substituted with 1 to 5 substituents selected from (1) halogen, (2) —OH, (3) C1-C4-alkyl, (4) C1-C4-alkoxy and (5) C1-C4-haloalkyl), or —NH—C(═O)—O—(C1-C4-alkyl) which may be optionally substituted with 1 to 5 substituents selected from (1) halogen, (2) —OH and (3) C1-C4-alkoxy, more preferably cyano, —NH—C(═O)-(methyl which may be optionally substituted with 1 to 3 substituents selected from (1) halogen, (2) —OH and (3) C1-C4-alkoxy), or —NH—C(═O)—O—(C1-C4-alkyl) which may be optionally substituted with 1 to 5 substituents selected from (1) halogen, (2) —OH and (3) C1-C4-alkoxy);
R⁹ is halogen, cyano, C1-C4-alkyl which may be optionally substituted with 1 to 5 substituents selected from (1) halogen, (2) —OH and (3) C1-C4-alkoxy, —C(═O)—(C1-C4-alkyl) which may be optionally substituted with 1 to 5 substituents selected from (1) halogen, (2) —OH and (3) C1-C4-alkoxy, —C(═O)—(C3-C7-cycloalkyl which may be optionally substituted with 1 to 5 substituents selected from (1) halogen, (2) —OH, (3) C1-C4-alkyl, (4) C1-C4-alkoxy and (5) C1-C4-haloalkyl), —C(═O)—(3 to 10 membered heterocycle which may be optionally substituted with 1 to 5 substituents selected from (1) halogen, (2) —OH, (3) C1-C4-alkyl, (4) C1-C4-alkoxy and (5) C1-C4-haloalkyl), —C(═O)—O—(C1-C4-alkyl) which may be optionally substituted with 1 to 5 substituents selected from (1) halogen, (2) —OH and (3) C1-C4-alkoxy, —C(═O)—NH—(C1-C4-alkyl) which may be optionally substituted with 1 to 5 substituents selected from (1) halogen, (2) —OH and (3) C1-C4-alkoxy, —C(═O)—NH—(C3-C7-cycloalkyl which may be optionally substituted with 1 to 5 substituents selected from (1) halogen, (2) —OH, (3) C1-C4-alkyl, (4) C1-C4-alkoxy and (5) C1-C4-haloalkyl), —NH—C(═O)—(C1-C4-alkyl) which may be optionally substituted with 1 to 5 substituents selected from (1) halogen, (2) —OH and (3) C1-C4-alkoxy, —NH—C(═O)—(C3-C7-cycloalkyl which may be optionally substituted with 1 to 5 substituents selected from (1) halogen, (2) —OH, (3) C1-C4-alkyl, (4) C1-C4-alkoxy and (5) C1-C4-haloalkyl), —NH—C(═O)—(3 to 10 membered heterocycle which may be optionally substituted with 1 to 5 substituents selected from (1) halogen, (2) —OH, (3) C1-C4-alkyl, (4) C1-C4-alkoxy and (5) C1-C4-haloalkyl), —NH—C(═O)—O—(C1-C4-alkyl) which may be optionally substituted with 1 to 5 substituents selected from (1) halogen, (2) —OH and (3) C1-C4-alkoxy, or —SO₂ (C1-C4-alkyl) which may be optionally substituted with 1 to 5 substituents selected from (1) halogen, (2) —OH and (3) C1-C4-alkoxy (preferably halogen, cyano, C1-C4-alkyl which may be optionally substituted with 1 to 5 substituents selected from (1) halogen, (2) —OH and (3) C1-C4-alkoxy, —C(═O)—(C1-C4-alkyl) which may be optionally substituted with 1 to 5 substituents selected from (1) halogen, (2) —OH and (3) C1-C4-alkoxy, —C(═O)—(C3-C7-cycloalkyl which may be optionally substituted with 1 to 5 substituents selected from (1) halogen, (2) —OH, (3) C1-C4-alkyl, (4) C1-C4-alkoxy and (5) C1-C4-haloalkyl), —C(═O)—(4 to 6 membered heterocycle which may be optionally substituted with 1 to 5 substituents selected from (1) halogen, (2) —OH, (3) C1-C4-alkyl, (4) C1-C4-alkoxy and (5) C1-C4-haloalkyl), —C(═O)—O—(C1-C4-alkyl) which may be optionally substituted with 1 to 5 substituents selected from (1) halogen, (2) —OH and (3) C1-C4-alkoxy, —C(═O)—NH—(C1-C4-alkyl) which may be optionally substituted with 1 to 5 substituents selected from (1) halogen, (2) —OH and (3) C1-C4-alkoxy, —NH—C(═O)—(C1-C4-alkyl) which may be optionally substituted with 1 to 5 substituents selected from (1) halogen, (2) —OH and (3) C1-C4-alkoxy, —NH—C(═O)—(C3-C7-cycloalkyl which may be optionally substituted with 1 to 5 substituents selected from (1) halogen, (2) —OH, (3) C1-C4-alkyl, (4) C1-C4-alkoxy and (5) C1-C4-haloalkyl), —NH—C(═O)—(4 to 6 membered heterocycle which may be optionally substituted with 1 to 5 substituents selected from (1) halogen, (2) —OH, (3) C1-C4-alkyl, (4) C1-C4-alkoxy and (5) C1-C4-haloalkyl), —NH—C(═O)—O—(C1-C4-alkyl) which may be optionally substituted with 1 to 5 substituents selected from (1) halogen, (2) —OH and (3) C1-C4-alkoxy, or —SO₂—(C1-C4-alkyl) which may be optionally substituted with 1 to 5 substituents selected from (1) halogen, (2) —OH and (3) C1-C4-alkoxy, more preferably halogen, cyano, methyl which may be optionally substituted with 1 to 3 substituents selected from (1) halogen, (2) —OH and (3) C1-C4-alkoxy, —C(═O)—(C1-C4-alkyl) which may be optionally substituted with 1 to 5 substituents selected from (1) halogen, (2) —OH and (3) C1-C4-alkoxy, —C(═O)—(C3-C7-cycloalkyl which may be optionally substituted with 1 to 5 substituents selected from (1) halogen, (2) —OH, (3) C1-C4-alkyl, (4) C1-C4-alkoxy and (5) C1-C4-haloalkyl), —C(═O)—(4 to 6 membered heterocycle which may be optionally substituted with 1 to 5 substituents selected from (1) halogen, (2) —OH, (3) C1-C4-alkyl, (4) C1-C4-alkoxy and (5) C1-C4-haloalkyl), —C(═O)—O—(C1-C4-alkyl) which may be optionally substituted with 1 to 5 substituents selected from (1) halogen, (2) —OH and (3) C1-C4-alkoxy, —NH—C(═O)—(C1-C4-alkyl) which may be optionally substituted with 1 to 5 substituents selected from (1) halogen, (2) —OH and (3) C1-C4-alkoxy, —NH—C(═O)—(C3-C7-cycloalkyl which may be optionally substituted with 1 to 5 substituents selected from (1) halogen, (2) —OH, (3) C1-C4-alkyl, (4) C1-C4-alkoxy and (5) C1-C4-haloalkyl), —NH—C(═O)—(4 to 6 membered heterocycle which may be optionally substituted with 1 to 5 substituents selected from (1) halogen, (2) —OH, (3) C1-C4-alkyl, (4) C1-C4-alkoxy and (5) C1-C4-haloalkyl), or —NH—C(═O)—O—(C1-C4-alkyl) which may be optionally substituted with 1 to 5 substituents selected from (1) halogen, (2) —OH and (3) C1-C4-alkoxy);
R¹⁰ is halogen, cyano, C1-C4-alkyl which may be optionally substituted with 1 to 5 substituents selected from (1) halogen, (2) —OH and (3) C1-C4-alkoxy, —C(═O)—(C1-C4-alkyl) which may be optionally substituted with 1 to 5 substituents selected from (1) halogen, (2) —OH and (3) C1-C4-alkoxy, —C(═O)—(C3-C7-cycloalkyl which may be optionally substituted with 1 to 5 substituents selected from (1) halogen, (2) —OH, (3) C1-C4-alkyl, (4) C1-C4-alkoxy and (5) C1-C4-haloalkyl), —C(═O)—(3 to 10 membered heterocycle which may be optionally substituted with 1 to 5 substituents selected from (1) halogen, (2) —OH, (3) C1-C4-alkyl, (4) C1-C4-alkoxy and (5) C1-C4-haloalkyl), —C(═O)—O—(C1-C4-alkyl) which may be optionally substituted with 1 to 5 substituents selected from (1) halogen, (2) —OH and (3) C1-C4-alkoxy, —C(═O)—NH—(C1-C4-alkyl) which may be optionally substituted with 1 to 5 substituents selected from (1) halogen, (2) —OH and (3) C1-C4-alkoxy, —C(═O)—NH—(C3-C7-cycloalkyl which may be optionally substituted with 1 to 5 substituents selected from (1) halogen, (2) —OH, (3) C1-C4-alkyl, (4) C1-C4-alkoxy and (5) C1-C4-haloalkyl), —NH—C(═O)—(C1-C4-alkyl) which may be optionally substituted with 1 to 5 substituents selected from (1) halogen, (2) —OH and (3) C1-C4-alkoxy, —NH—C(═O)—(C3-C7-cycloalkyl which may be optionally substituted with 1 to 5 substituents selected from (1) halogen, (2) —OH, (3) C1-C4-alkyl, (4) C1-C4-alkoxy and (5) C1-C4-haloalkyl), —NH—C(═O)—(3 to 10 membered heterocycle which may be optionally substituted with 1 to 5 substituents selected from (1) halogen, (2) —OH, (3) C1-C4-alkyl, (4) C1-C4-alkoxy and (5) C1-C4-haloalkyl), —NH—C(═O)—O—(C1-C4-alkyl) which may be optionally substituted with 1 to 5 substituents selected from (1) halogen, (2) —OH and (3) C1-C4-alkoxy, or —SO₂—(C1-C4-alkyl) which may be optionally substituted with 1 to 5 substituents selected from (1) halogen, (2) —OH and (3) C1-C4-alkoxy (preferably halogen, cyano, C1-C4-alkyl which may be optionally substituted with 1 to 5 substituents selected from (1) halogen, (2) —OH and (3) C1-C4-alkoxy, —C(═O)—(C1-C4-alkyl) which may be optionally substituted with 1 to 5 substituents selected from (1) halogen, (2) —OH and (3) C1-C4-alkoxy, —C(═O)—(C3-C7-cycloalkyl which may be optionally substituted with 1 to 5 substituents selected from (1) halogen, (2) —OH, (3) C1-C4-alkyl, (4) C1-C4-alkoxy and (5) C1-C4-haloalkyl), —C(═O)—(4 to 6 membered heterocycle which may be optionally substituted with 1 to 5 substituents selected from (1) halogen, (2) —OH, (3) C1-C4-alkyl, (4) C1-C4-alkoxy and (5) C1-C4-haloalkyl), —C(═O)—O—(C1-C4-alkyl) which may be optionally substituted with 1 to 5 substituents selected from (1) halogen, (2) —OH and (3) C1-C4-alkoxy, —C(═O)—NH—(C1-C4-alkyl) which may be optionally substituted with 1 to 5 substituents selected from (1) halogen, (2) —OH and (3) C1-C4-alkoxy, —NH—C(═O)—(C1-C4-alkyl) which may be optionally substituted with 1 to 5 substituents selected from (1) halogen, (2) —OH and (3) C1-C4-alkoxy, —NH—C(═O)—(C3-C7-cycloalkyl which may be optionally substituted with 1 to 5 substituents selected from (1) halogen, (2) —OH, (3) C1-C4-alkyl, (4) C1-C4-alkoxy and (5) C1-C4-haloalkyl), —NH—C(═O)—(4 to 6 membered heterocycle which may be optionally substituted with 1 to 5 substituents selected from (1) halogen, (2) —OH, (3) C1-C4-alkyl, (4) C1-C4-alkoxy and (5) C1-C4-haloalkyl), —NH—C(═O)—O—(C1-C4-alkyl) which may be optionally substituted with 1 to 5 substituents selected from (1) halogen, (2) —OH and (3) C1-C4-alkoxy, or —SO₂—(C1-C4-alkyl) which may be optionally substituted with 1 to 5 substituents selected from (1) halogen, (2) —OH and (3) C1-C4-alkoxy, more preferably halogen, cyano, or C1-C4-alkyl which may be optionally substituted with 1 to 5 substituents selected from (1) halogen, (2) —OH and (3) C1-C4-alkoxy);
R¹¹ is halogen, cyano, C1-C4-alkyl which may be optionally substituted with 1 to 5 substituents selected from (1) halogen, (2) —OH and (3) C1-C4-alkoxy, —C(═O)—(C1-C4-alkyl) which may be optionally substituted with 1 to 5 substituents selected from (1) halogen, (2) —OH and (3) C1-C4-alkoxy, —C(═O)—(C3-C7-cycloalkyl which may be optionally substituted with 1 to 5 substituents selected from (1) halogen, (2) —OH, (3) C1-C4-alkyl, (4) C1-C4-alkoxy and (5) C1-C4-haloalkyl), —C(═O)—(3 to 10 membered heterocycle which may be optionally substituted with 1 to 5 substituents selected from (1) halogen, (2) —OH, (3) C1-C4-alkyl, (4) C1-C4-alkoxy and (5) C1-C4-haloalkyl), —C(═O)—O—(C1-C4-alkyl) which may be optionally substituted with 1 to 5 substituents selected from (1) halogen, (2) —OH and (3) C1-C4-alkoxy, —C(═O)—NH—(C1-C4-alkyl) which may be optionally substituted with 1 to 5 substituents selected from (1) halogen, (2) —OH and (3) C1-C4-alkoxy, —C(═O)—NH—(C3-C7-cycloalkyl which may be optionally substituted with 1 to 5 substituents selected from (1) halogen, (2) —OH, (3) C1-C4-alkyl, (4) C1-C4-alkoxy and (5) C1-C4-haloalkyl), —NH—C(═O)—(C1-C4-alkyl) which may be optionally substituted with 1 to 5 substituents selected from (1) halogen, (2) —OH and (3) C1-C4-alkoxy, —NH—C(═O)—(C3-C7-cycloalkyl which may be optionally substituted with 1 to 5 substituents selected from (1) halogen, (2) —OH, (3) C1-C4-alkyl, (4) C1-C4-alkoxy and (5) C1-C4-haloalkyl), —NH—C(═O)—(3 to 10 membered heterocycle which may be optionally substituted with 1 to 5 substituents selected from (1) halogen, (2) —OH, (3) C1-C4-alkyl, (4) C1-C4-alkoxy and (5) C1-C4-haloalkyl), —NH—C(═O)—O—(C1-C4-alkyl) which may be optionally substituted with 1 to 5 substituents selected from (1) halogen, (2) —OH and (3) C1-C4-alkoxy, or —SO₂ (C1-C4-alkyl) which may be optionally substituted with 1 to 5 substituents selected from (1) halogen, (2) —OH and (3) C1-C4-alkoxy (preferably halogen, cyano, C1-C4-alkyl which may be optionally substituted with 1 to 5 substituents selected from (1) halogen, (2) —OH and (3) C1-C4-alkoxy, —C(═O)—(C1-C4-alkyl) which may be optionally substituted with 1 to 5 substituents selected from (1) halogen, (2) —OH and (3) C1-C4-alkoxy, —C(═O)—(C3-C7-cycloalkyl which may be optionally substituted with 1 to 5 substituents selected from (1) halogen, (2) —OH, (3) C1-C4-alkyl, (4) C1-C4-alkoxy and (5) C1-C4-haloalkyl), —C(═O)—(4 to 6 membered heterocycle which may be optionally substituted with 1 to 5 substituents selected from (1) halogen, (2) —OH, (3) C1-C4-alkyl, (4) C1-C4-alkoxy and (5) C1-C4-haloalkyl), —C(═O)—O—(C1-C4-alkyl) which may be optionally substituted with 1 to 5 substituents selected from (1) halogen, (2) —OH and (3) C1-C4-alkoxy, —C(═O)—NH—(C1-C4-alkyl) which may be optionally substituted with 1 to 5 substituents selected from (1) halogen, (2) —OH and (3) C1-C4-alkoxy, —NH—C(═O)—(C1-C4-alkyl) which may be optionally substituted with 1 to 5 substituents selected from (1) halogen, (2) —OH and (3) C1-C4-alkoxy, —NH—C(═O)—(C3-C7-cycloalkyl which may be optionally substituted with 1 to 5 substituents selected from (1) halogen, (2) —OH, (3) C1-C4-alkyl, (4) C1-C4-alkoxy and (5) C1-C4-haloalkyl), —NH—C(═O)—(4 to 6 membered heterocycle which may be optionally substituted with 1 to 5 substituents selected from (1) halogen, (2) —OH, (3) C1-C4-alkyl, (4) C1-C4-alkoxy and (5) C1-C4-haloalkyl), —NH—C(═O)—O—(C1-C4-alkyl) which may be optionally substituted with 1 to 5 substituents selected from (1) halogen, (2) —OH and (3) C1-C4-alkoxy, or —SO₂—(C1-C4-alkyl) which may be optionally substituted with 1 to 5 substituents selected from (1) halogen, (2) —OH and (3) C1-C4-alkoxy, more preferably halogen, cyano or C1-C4-alkyl which may be optionally substituted with 1 to 5 substituents selected from (1) halogen, (2) —OH and (3) C1-C4-alkoxy); R is 6 to 10 membered aryl which may be optionally substituted with 1 to 5 R⁶ or 5 to 10 membered heteroaryl which may be optionally substituted with 1 to 5 R⁶ (preferably 6 to 10 membered aryl or 5 membered heteroaryl which may be optionally substituted with 1 to 3 R⁶, more preferably thiophenyl or phenyl which may be optionally substituted with 1 to 3 R⁶, most preferably phenyl which may be optionally substituted with 1 to 3 R⁶);
R⁶ is halogen, cyano, C1-C4-alkyl which may be optionally substituted with 1 to 5 halogen or C1-C4-alkoxy which may be optionally substituted with 1 to 5 halogen (preferably halogen, C1-C4-alkyl which may be optionally substituted with 1 to 5 halogen, C1-C4-alkoxy which may be optionally substituted with 1 to 5 halogen, more preferably halogen or methyl which may be optionally substituted with 1 to 3 halogen), and multiple R⁶, R⁸, R⁹, R¹⁰ or R¹¹ may be the same as or different from each other.

[4-2] The compound or a pharmaceutically acceptable salt thereof according to [3], wherein R² is halogen.

[5] The compound or a pharmaceutically acceptable salt thereof according to [3], which is a compound of formula (Ia-2):

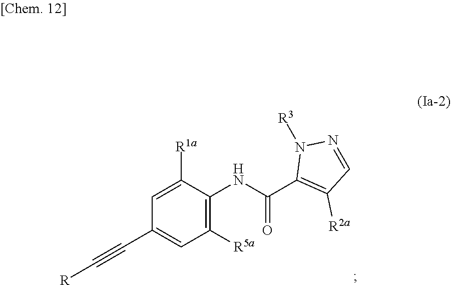

wherein
R^{1a} is selected from (1) halogen and (2) C1-C10-alkyl (preferably C1-4 alkyl);
R^{5a} is selected from (1)hydrogen, (2)halogen and (3) C1-C10-alkyl (preferably C1-4 alkyl);
R^{2a} is halogen;
and the other symbols are as defined in [1].

[6] The compound or a pharmaceutically acceptable salt thereof according to [5], which is a compound of formula (Ia-3):

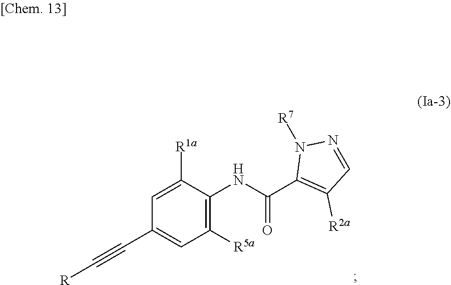

wherein all symbols are as defined in [1] or [5].

[7] The compound or a pharmaceutically acceptable salt thereof according to [5], which is a compound of formula (Ia-4):

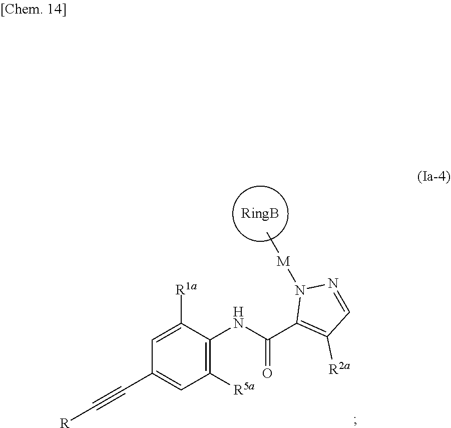

wherein all symbols are as defined in [1] or [5].

[8] The compound or a pharmaceutically acceptable salt thereof according to [3], which is a compound of formula (Ia-5):

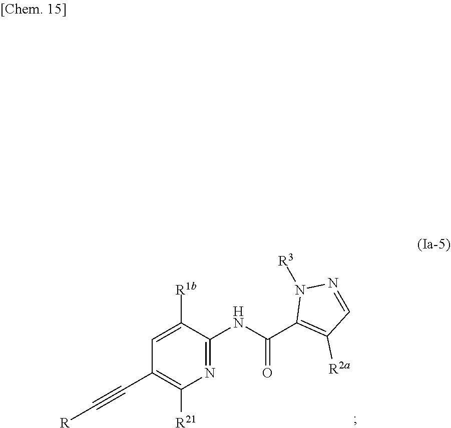

wherein R^{1b} is selected from (1) halogen and (2) C1-C10-alkyl (preferably C1-4 alkyl); and the other symbols are as defined in [1] or [5].

[9] The compound or a pharmaceutically acceptable salt thereof according to [8], which is a compound of formula (Ia-6):

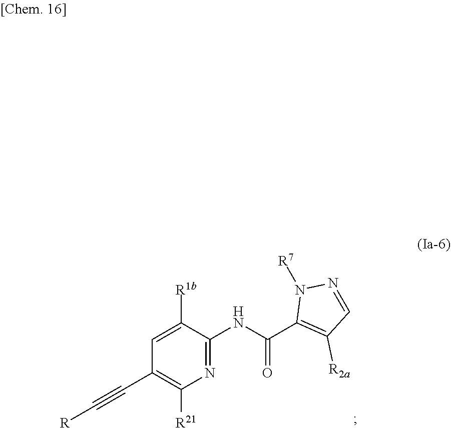

wherein all symbols are as defined in [1], [5] or [8].

[10] The compound or a pharmaceutically acceptable salt thereof according to [8], which is a compound of formula (Ia-7):

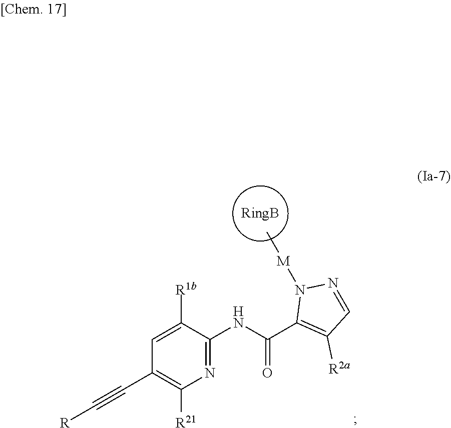

wherein all symbols are as defined in [1], [5] or [8].

[11] The compound or a pharmaceutically acceptable salt thereof according to any one of [1] to [10], wherein R is phenyl which may be optionally substituted with 1 to 5 R⁶.

[12] The compound or a pharmaceutically acceptable salt thereof according to [2], which is a compound of formula (Ia-1-7b):

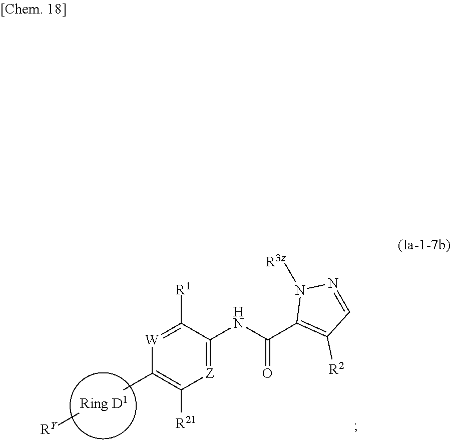

wherein R^{Y} is phenyl which may be optionally substituted with 1 to 5 R⁶;
Ring D¹ is selected from (1) benzene and (2) 5 membered heteroaryl;
R^{3z} is

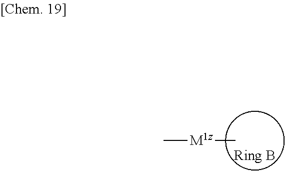

wherein M^{1z} is selected from (1) bond or (2) C1-C4-alkylene which may be optionally substituted with 1 to 3 halogens;
and the other symbols are as defined in [1].

[13] The compound according to [12], wherein Ring D¹ is selected from

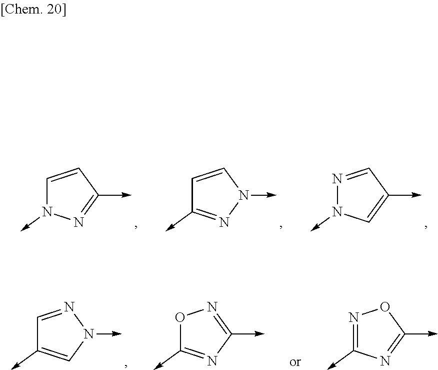

wherein right arrow is connecting position with 6 membered ring and left arrow is connecting position with R^{Y}.

[14] The compound or a pharmaceutically acceptable salt thereof according to [2], which is a compound of formula (Ia-1-2b):

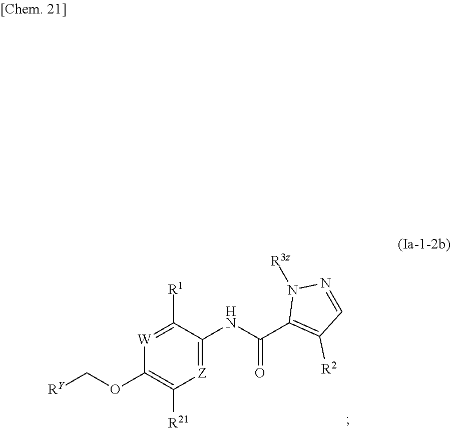

wherein all symbols are as defined in [1] or [12].

[15] The compound or a pharmaceutically acceptable salt thereof according to [1], which is a compound of formula (Ib):

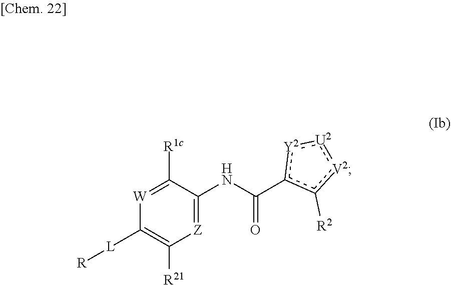

wherein

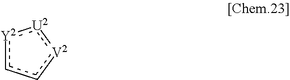

is selected from (1) 1,2,3-triazole substituted with R³, (2) pyrrole substituted with R³, (3) pyrazole substituted with R³ and (4) imidazole substituted with R³;
wherein Y² is selected from (1) CH and (2) N;
U² is selected from (1) CR³ and (2) NR³;
V² is selected from (1) CH, (2) CR^{x} and (3) N;
R^{1c} is selected from (1) C1-C10-alkyl, (2) halogen and (3) C1-C10-haloalkyl;
and the other symbols are as defined in [1].

[16] The compound or a pharmaceutically acceptable salt thereof according to [15], which is a compound of formula (Ib-1):

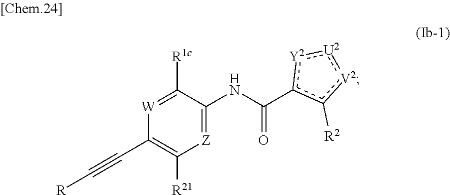

wherein all symbols are as defined [1] or [15].

[17] The compound or a pharmaceutically acceptable salt thereof according to [16], which is a compound of formula (Ib-2):

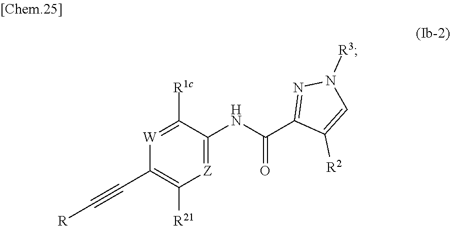

wherein all symbols are as defined [1] or [15].

[18] The compound or a pharmaceutically acceptable salt thereof according to [17], wherein R² is halogen.

[19] The compound or a pharmaceutically acceptable salt thereof according to [18], which is a compound of formula (Ib-3):

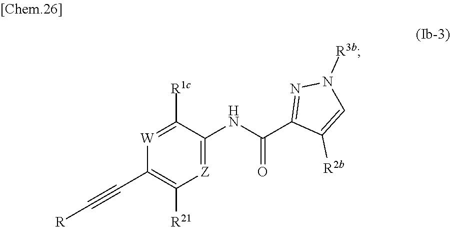

wherein R^{2b} is halogen;
R^{3b} is

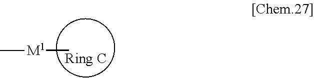

wherein M¹ is selected from (1) bond and (2) C1-C10-alkylene which may be optionally substituted with 1 to 3 halogens,
Ring C is selected from (1) C3-C10-cycloalkyl which may be optionally substituted with 1 to 5 R¹³, (2) 3 to 15 membered heterocycle which may be optionally substituted with 1 to 5 R¹⁴ and (3) 5 to 15 membered heteroaryl which may be optionally substituted with 1 to 5 R¹⁵;
wherein multiple R¹³, R¹⁴ or R¹⁵ may be the same as or different from each other;
R¹³, R¹⁴ and R¹⁵ are each independently selected from (1) halogen, (2) C1-C10-alkyl, (3) —C(═O)—(C1-C10-haloalkyl), (4) cyano, (5) —C(═O)—(C1-C10-alkyl), (6) —C(═O)—(C3-C10-cycloalkyl), (7) —C(═O)—(3 to 15 membered heterocycle) and (8)oxo; the other symbols are defined as [1] or [15].

[20] The compound or a pharmaceutically acceptable salt thereof according to any one of [15] to [19], wherein R is phenyl which may be optionally substituted with 1 to 5 R⁶.

[21] The compound or a pharmaceutically acceptable salt thereof according to [1], wherein the compound is
(1) 1-((1,4-dioxan-2-yl)methyl)-4-chloro-N-(3-methyl-5-(phenylethynyl)pyridin-2-yl)-1H-pyrazole-5-carboxamide,
(2) 4-chloro-N-(3-methyl-5-(phenylethynyl)pyridin-2-yl)-1-((tetrahydro-2H-pyran-4-yl)methyl)-1H-pyrazole-5-carboxamide,
(3) (R)-4-chloro-N-(3-methyl-5-(phenylethynyl)pyridin-2-yl)-1-(tetrahydrofuran-3-yl)-1H-pyrazole-5-carboxamide,
(4) (S)-1-((1,4-dioxan-2-yl)methyl)-4-chloro-N-(3-fluoro-5-(phenylethynyl)pyridin-2-yl)-1H-pyrazole-5-carboxamide,
(5) 1-(1-acetylpiperidin-4-yl)-4-chloro-N-(3-methyl-5-(phenylethynyl)pyridin-2-yl)-1H-pyrazole-5-carboxamide,
(6) 1-(1-acetylpiperidin-4-yl)-4-chloro-N-(3-fluoro-5-(phenylethynyl)pyridin-2-yl)-1H-pyrazole-5-carboxamide,
(7) 1-(1-acetylpiperidin-4-yl)-4-chloro-N-(3-fluoro-5-((4-fluorophenyl)ethynyl)pyridin-2-yl)-1H-pyrazole-5-carboxamide,
(8) 1-(1-acetylpiperidin-4-yl)-4-chloro-N-(5-((4-fluorophenyl)ethynyl)-3-methylpyridin-2-yl)-1H-pyrazole-5-carboxamide,
(9) 4-chloro-N-(3-methyl-5-(phenylethynyl)pyridin-2-yl)-1-(4,5,6,7-tetrahydropyrazolo[1,5-a]pyridin-5-yl)-1H-pyrazole-5-carboxamide,
(10) 1-((3R,4R)-1-acetyl-3-fluoropiperidin-4-yl)-4-chloro-N-(5-((4-fluorophenyl)ethynyl)-3-methylpyridin-2-yl)-1H-pyrazole-5-carboxamide,
(11) 1-(1-(1,4-dioxane-2-carbonyl)piperidin-4-yl)-4-chloro-N-(3-fluoro-5-(phenylethynyl)pyridin-2-yl)-1H-pyrazole-5-carboxamide,
(12) 1-(cis-4-acetamidocyclohexyl)-4-chloro-N-(3-methyl-5-(phenylethynyl)pyridin-2-yl)-1H-pyrazole-5-carboxamide,
(13) 1-(cis-4-acetamidocyclohexyl)-4-chloro-N-(3-fluoro-5-(phenylethynyl)pyridin-2-yl)-1H-pyrazole-5-carboxamide,
(14) 1-(cis-4-acetamidocyclohexyl)-4-chloro-N-(5-((4-fluorophenyl)ethynyl)-3-methylpyridin-2-yl)-1H-pyrazole-5-carboxamide,
(15) 1-((1-acetyl-4-fluoropiperidin-4-yl)methyl)-4-chloro-N-(3-methyl-5-(phenylethynyl)pyridin-2-yl)-1H-pyrazole-5-carboxamide,
(16) (S)-1-(1-(1-acetylpiperidin-4-yl)ethyl)-4-chloro-N-(3-methyl-5-(phenylethynyl)pyridin-2-yl)-1H-pyrazole-5-carboxamide,
(17) 1-((1-acetyl-3-fluoroazetidin-3-yl)methyl)-4-chloro-N-(3-fluoro-5-(phenylethynyl)pyridin-2-yl)-1H-pyrazole-5-carboxamide,
(18) 1-((1-acetyl-3-fluoroazetidin-3-yl)methyl)-4-chloro-N-(3-methyl-5-(phenylethynyl)pyridin-2-yl)-1H-pyrazole-5-carboxamide,
(19) 4-chloro-1-[(3S)-1-(cyclopropanecarbonyl)pyrrolidin-3-yl]-N-[3-methyl-5-(phenylethynyl)pyridin-2-yl]-1H-pyrazole-5-carboxamide,
(20) 4-chloro-1-[(3R)-1-(cyclopropanecarbonyl)pyrrolidin-3-yl]-N-[3-methyl-5-(phenylethynyl)pyridin-2-yl]-1H-pyrazole-5-carboxamide.

[22] A pharmaceutical composition comprising a compound of formula (I), or a pharmaceutically acceptable salt thereof, and a pharmaceutically acceptable carrier.

[23] The pharmaceutical composition according to [22], wherein the compound of formula (I) is a compound of formula (Ia).

[24] The pharmaceutical composition according to [23], which is a TREK1, TREK2 or both TREK1 and TREK2 inhibitor.

[25] The pharmaceutical composition according to [22], wherein the compound of formula (I) is a compound of formula (Ib).

[26] The pharmaceutical composition according to [25], which is a TREK1, TREK2 or both TREK1 and TREK2 activator.

[27] The pharmaceutical composition according to [23], which is a preventive and/or therapeutic agent for a disorder associated with TREK1, TREK2 or dual TREK1/TREK2 dysfunction in which inhibitors of TREK1, TREK2 or both TREK1 and TREK2 would offer therapeutic benefit.

[28] The pharmaceutical composition according to [27], wherein the disorder associated with TREK1, TREK2 or dual TREK1/TREK2 dysfunction in which inhibitors of TREK1, TREK2 or both TREK1 and TREK2 would offer therapeutic benefit is a neurological and/or psychiatric disorder.

[29] The pharmaceutical composition according to [28], wherein the neurological and/or psychiatric disorder is selected from depression, schizophrenia, anxiety disorders, bipolar disorder, Alzheimer's disease, Parkinson's disease, Huntington's disease, Amyotrophic lateral sclerosis, 22q11.2 deletion syndrome, neuropathic pain or cerebral infarction.

[30] The pharmaceutical composition according to [25], which is a preventive and/or therapeutic agent for a disorder associated with TREK1, TREK2 or dual TREK1/TREK2 dysfunction in which activators of TREK1, TREK2 or both TREK1 and TREK2 would offer therapeutic benefit.

[31] The pharmaceutical composition according to [30], wherein the disorder associated with TREK1, TREK2 or dual TREK1/TREK2 dysfunction in which activators of TREK1, TREK2 or both TREK1 and TREK2 would offer therapeutic benefit is selected from pain, migraine, nasal inflammation, atrial fibrillation, acute respiratory distress syndrome, acute lung injury, overactive bladder, cerebral ischemia, epilepsy, amyotrophic lateral sclerosis, neuronal degenerative diseases (e.g. Alzheimer's disease), sepsis, pancreatic cancer, Cushing's syndrome, autosomal dominant polycystic kidney disease, bone fracture, osteoporosis, temporal lobe epilepsy, schizophrenia, colitis, or addiction.

[32] A medicament comprising the compound of formula (Ta), or a pharmaceutically acceptable salt thereof with at least one selected from typical antipsychotics and atypical antipsychotics.

[33] A medicament comprising the compound of formula (Ta), or a pharmaceutically acceptable salt thereof with at least one selected from benzodiazepine antianxiety drug, thienodiazepine antianxiety drug, non-benzodiazepine antianxiety drug, CRF antagonist, neurokinin-1 (NK1) antagonist, tricyclic antidepressant, tetracyclic antidepressant, monoamine oxidase (MAO) inhibitor, triazolopyridine antidepressant, serotonin and noradrenaline reuptake inhibitor (SNRI), selective serotonin reuptake inhibitor (SSRI), serotonin reuptake inhibitor, noradrenergic and specific serotonergic antidepressant (NaSSA), noradrenaline and dopamine disinhibition drug (NDDI), selective serotonin reuptake enhancer (SSRE).

[34] A medicament comprising the compound of formula (Ib), or a pharmaceutically acceptable salt thereof with at least one selected from an alkylating agent, an antimetabolite, an anti-cancer antibiotic, a plant-based preparation, a hormonal agent, a platinum compound, a topoisomerase inhibitor, a kinase inhibitor, an anti-CD20 antibody, an anti-HER2 antibody, an anti-EGFR antibody, an anti-VEGF antibody, a proteasome inhibitor, an HDAC inhibitor, an immune-checkpoint inhibitor and immunomodulator.

[35] A method for preventing and/or treating a disorder associated with TREK1, TREK2 or dual TREK1/TREK2 dysfunction in which inhibitors of TREK1, TREK2 or both TREK1 and TREK2 would offer therapeutic benefit in a mammal, comprising a step of administering to the mammal in need thereof a therapeutically effective amount a compound of any one of [2] to [14] and [21], or pharmaceutically acceptable salt thereof.

[36] The method according to [35], wherein a disorder associated with TREK1, TREK2 or dual TREK1/TREK2 dysfunction in which inhibitors of TREK1, TREK2 or both TREK1 and TREK2 would offer therapeutic benefit in a mammal is a neurological and/or psychiatric disorder associated with TREK1, TREK2 or dual TREK1/TREK2 dysfunction in which inhibitors of TREK1, TREK2 or both TREK1 and TREK2 would offer therapeutic benefit in a mammal.

[37] The method according to [36], wherein the neurological and/or psychiatric disorder associated with TREK1, TREK2 or dual TREK1/TREK2 dysfunction in which inhibitors of TREK1, TREK2 or both TREK1 and TREK2 would offer therapeutic benefit is selected from depression, schizophrenia, anxiety disorders, bipolar disorder, Alzheimer's disease, Parkinson's disease, Huntington's disease, Amyotrophic lateral sclerosis, 22q11.2 deletion syndrome, neuropathic pain or cerebral infarction.

[38] A method for preventing and/or treating a disorder associated with TREK1, TREK2 or dual TREK1/TREK2 dysfunction in which activators of TREK1, TREK2 or both TREK1 and TREK2 would offer therapeutic benefit, comprising a step of administering to a mammal in need thereof a therapeutically effective amount a compound of any one of claims [15] to [20] or a pharmaceutically acceptable salt thereof.

[39] The method according to [38], wherein the disorder associated with TREK1, TREK2 or dual TREK1/TREK2 dysfunction in which activators of TREK1, TREK2 or both TREK1 and TREK2 would offer therapeutic benefit is selected from pain, migraine, nasal inflammation, atrial fibrillation, acute respiratory distress syndrome, acute lung injury, overactive bladder, cerebral ischemia, epilepsy, amyotrophic lateral sclerosis, neuronal degenerative diseases (e.g. Alzheimer's disease), sepsis, pancreatic cancer, Cushing's syndrome, autosomal dominant polycystic kidney disease, bone fracture, osteoporosis, temporal lobe epilepsy, schizophrenia, colitis, or addiction.

[40] A compound of formula (Ia), or a pharmaceutically acceptable salt thereof for use in preventing and/or treating of a neurological and/or psychiatric disorder associated with TREK1, TREK2 or dual TREK1/TREK2 dysfunction in which inhibitors of TREK1, TREK2 or both TREK1 and TREK2 would offer therapeutic benefit.

[41] The compound of formula (Ib), or a pharmaceutically acceptable salt thereof for use in preventing and/or treating of a disorder associated with TREK1, TREK2 or dual TREK1/TREK2 dysfunction in which activators of TREK1, TREK2 or both TREK1 and TREK2 would offer therapeutic benefit.

[42] Use of the compound of formula (Ta), or a pharmaceutically acceptable salt thereof for production of a preventive and/or therapeutic agent against a neurological and/or psychiatric disorder associated with TREK1, TREK2 or dual TREK1/TREK2 dysfunction in which inhibitors of TREK1, TREK2 or both TREK1 and TREK2 would offer therapeutic benefit.

[43] Use of the compound of formula (Tb), or a pharmaceutically acceptable salt thereof for production of a preventive and/or therapeutic agent against a disorder associated with TREK1, TREK2 or dual TREK1/TREK2 dysfunction in which activators of TREK1, TREK2 or both TREK1 and TREK2 would offer therapeutic benefit.

[44] A kit comprising a compound or a pharmaceutically acceptable salt thereof according to any one of [2] to [14] or [21], and one or more of: (a) at least one agent known to decrease TREK1 channel activity; (b) at least one agent known to decrease TREK2 channel activity; (c) at least one agent known to prevent and/or treat a disorder associated with TREK channel dysfunction in which inhibitors of TREK1, TREK2 or both TREK1/TREK2 would offer therapeutic benefit in a mammal; (d) instructions for preventing and/or treating a disorder associated with TREK dysfunction in which inhibitors of TREK1, TREK2 or both TREK1/TREK2 would offer therapeutic benefit in a mammal; and (e) instructions for administering the compound in connection with cognitive behavioral therapy.

[45] A kit comprising a compound or a pharmaceutically acceptable salt thereof according to any one of [15] to [20], and one or more of: (a) at least one agent known to increase TREK1 channel activity; (b) at least one agent known to increase TREK2 channel activity; (c) at least one agent known to prevent and/or treat a disorder associated with TREK channel activity in which activators of TREK1, TREK2 or both TREK1/TREK2 would offer therapeutic benefit in a mammal; and (d) instructions for preventing and/or treating a disorder associated with TREK activity in which activators of TREK1, TREK2 or both TREK1/TREK2 would offer therapeutic benefit in a mammal.

Disclosed herein are modulators, especially inhibitors or activators, of the TREK (TWIK RElated K⁺ channels)-subtypes 1 and 2 (TREK1 and TREK2), methods of making same, pharmaceutical compositions comprising same, and methods of preventing and/or treating neurological, psychiatric, inflammatory, respiratory, renal and cardiovascular disorders associated with TREK channel dysfunction using same. The compounds include, but not limited to,
(1) 1-((1,4-dioxan-2-yl)methyl)-4-chloro-N-(3-methyl-5-(phenylethynyl)pyridin-2-yl)-1H-pyrazole-5-carboxamide,
(2) 4-chloro-N-(3-methyl-5-(phenylethynyl)pyridin-2-yl)-1-((tetrahydro-2H-pyran-4-yl)methyl)-1H-pyrazole-5-carboxamide,
(3) (R)-4-chloro-N-(3-methyl-5-(phenylethynyl)pyridin-2-yl)-1-(tetrahydrofuran-3-yl)-1H-pyrazole-5-carboxamide
(4) (S)-1-((1,4-dioxan-2-yl)methyl)-4-chloro-N-(3-fluoro-5-(phenylethynyl)pyridin-2-yl)-1H-pyrazole-5-carboxamide,
(5) 1-(1-acetylpiperidin-4-yl)-4-chloro-N-(3-methyl-5-(phenylethynyl)pyridin-2-yl)-1H-pyrazole-5-carboxamide,
(6) 1-(1-acetylpiperidin-4-yl)-4-chloro-N-(3-fluoro-5-(phenylethynyl)pyridin-2-yl)-1H-pyrazole-5-carboxamide,
(7) 1-(1-acetylpiperidin-4-yl)-4-chloro-N-(3-fluoro-5-((4-fluorophenyl)ethynyl)pyridin-2-yl)-1H-pyrazole-5-carboxamide,
(8) 1-(1-acetylpiperidin-4-yl)-4-chloro-N-(5-((4-fluorophenyl)ethynyl)-3-methylpyridin-2-yl)-1H-pyrazole-5-carboxamide,
(9) 4-chloro-N-(3-methyl-5-(phenylethynyl)pyridin-2-yl)-1-(4,5,6,7-tetrahydropyrazolo[1,5-a]pyridin-5-yl)-1H-pyrazole-5-carboxamide,
(10) 1-((3R,4R)-1-acetyl-3-fluoropiperidin-4-yl)-4-chloro-N-(5-((4-fluorophenyl)ethynyl)-3-methylpyridin-2-yl)-1H-pyrazole-5-carboxamide,
(11) 1-(1-(1,4-dioxane-2-carbonyl)piperidin-4-yl)-4-chloro-N-(3-fluoro-5-(phenylethynyl)pyridin-2-yl)-1H-pyrazole-5-carboxamide,
(12) 1-(cis-4-acetamidocyclohexyl)-4-chloro-N-(3-methyl-5-(phenylethynyl)pyridin-2-yl)-1H-pyrazole-5-carboxamide,
(13) 1-(cis-4-acetamidocyclohexyl)-4-chloro-N-(3-fluoro-5-(phenylethynyl)pyridin-2-yl)-1H-pyrazole-5-carboxamide,
(14) 1-(cis-4-acetamidocyclohexyl)-4-chloro-N-(5-((4-fluorophenyl)ethynyl)-3-methylpyrid in-2-yl)-1H-pyrazole-5-carboxamide,
(15) 1-((1-acetyl-4-fluoropiperidin-4-yl)methyl)-4-chloro-N-(3-methyl-5-(phenylethynyl)pyridin-2-yl)-1H-pyrazole-5-carboxamide,
(16) (S)-1-(1-(1-acetylpiperidin-4-yl)ethyl)-4-chloro-N-(3-methyl-5-(phenylethynyl)pyridin-2-yl)-1H-pyrazole-5-carboxamide,
(17) 1-((1-acetyl-3-fluoroazetidin-3-yl)methyl)-4-chloro-N-(3-fluoro-5-(phenylethynyl)pyridin-2-yl)-1H-pyrazole-5-carboxamide,
(18) 1-((1-acetyl-3-fluoroazetidin-3-yl)methyl)-4-chloro-N-(3-methyl-5-(phenylethynyl)pyridin-2-yl)-1H-pyrazole-5-carboxamide,
(19) 4-chloro-1-[(3S)-1-(cyclopropanecarbonyl)pyrrolidin-3-yl]-N-[3-methyl-5-(phenylethynyl)pyridin-2-yl]-1H-pyrazole-5-carboxamide,
(20) 4-chloro-1-[(3R)-1-(cyclopropanecarbonyl)pyrrolidin-3-yl]-N-[3-methyl-5-(phenylethynyl)pyridin-2-yl]-1H-pyrazole-5-carboxamide, or a pharmaceutically acceptable salt thereof.

### Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modem Methods of Organic Synthesis, 3^{rd} Edition, Cambridge University Press, Cambridge, 1987; the entire contents of each of which are incorporated herein by reference.

In some instances, the number of carbon atoms in a hydrocarbyl substituent (e.g., alkyl, alkoxy, alkenyl, alkenylene, alkynyl, alkylene, alkynylene, cycloalkyl, cycloalkane, haloalkyl, haloalkoxy, heteroalkyl or thioalkyl) is indicated by the prefix “Cx-Cy-”, wherein x is the minimum and y is the maximum number of carbon atoms in the substituent. Thus, for example, “C1-C3-alkyl” refers to an alkyl substituent containing from 1 to 3 carbon atoms, “C1-C10-alkoxy” refers to an alkoxy substituent containing from 1 to 10 carbon atoms, “C2-C10-alkenyl” refers to an alkenyl substituent containing from 2 to 10 carbon atoms, “C2-C4-alkenylene” refers to an alkenylene substituent containing from 2 to 4 carbon atoms, “C2-C10-alkynyl” refers to an alkynyl substituent containing from 2 to 10 carbon atoms, “C2-C10-alkylene” refers to an alkylene substituent containing from 2 to 10 carbon atoms, “C2-C4-alkynylene” refers to an alkynylene substituent containing from 2 to 4 carbon atoms, “C3-C10-cycloalkyl” refers to a cycloalkyl substituent containing from 3 to 10 carbon atoms, “C3-C10-cycloalkane” refers to a cycloalkane containing from 3 to 10 carbon atoms, “C1-C10-haloalkyl” refers to a haloalkyl substituent containing from 1 to 10 carbon atoms, “C1-C10-haloalkoxy” refers to a haloalkoxy substituent containing from 1 to 10 carbon atoms, “C2-C10-heteroalkyl” refers to a heteroalkyl substituent containing from 2 to 10 carbon atoms or “C1-C10-thioalkyl” refers to a thioalkyl substituent containing from 2 to 10 carbon atoms.

The term “alkyl,” as used herein, means a straight or branched, saturated hydrocarbon chain containing from 1 to 10 carbon atoms. The term “lower alkyl” or “C1-C6-alkyl” means a straight or branched chain hydrocarbon containing from 1 to 6 carbon atoms. The term “C1-C4-alkyl” means a straight or branched chain hydrocarbon containing from 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

As C1-C10 alkyl of R⁴, C1-C4-alkyl is preferred. More preferable C1-C10-alkyl of R⁴ is methyl.

As C1-C10-alkyl of substituents in R⁴, C1-C4-alkyl is preferred.

As C1-C10 alkyl of R⁵, C1-C4-alkyl is preferred. More preferable C1-C10-alkyl of R⁵ is methyl.

As C1-C10-alkyl of substituents in R⁵, C1-C4-alkyl is preferred.

As C1-C10 alkyl of R^{b}, C1-C4-alkyl is preferred.

As C1-C10 alkyl of R^{c}, C1-C4-alkyl is preferred.

As C1-C10 alkyl of R^{d}, C1-C4-alkyl is preferred.

As C1-C10-alkyl of substituents in Q, C1-C4-alkyl is preferred.

As C1-C10-alkyl of R²⁰¹, C1-C4-alkyl is preferred.

As C1-C10-alkyl of R²⁰², C1-C4-alkyl is preferred.

As C1-C10-alkyl of R²⁰³, C1-C4-alkyl is preferred.

As C1-C10-alkyl of R²⁰⁴, C1-C4-alkyl is preferred.

As C1-C10-alkyl of R²⁰⁵, C1-C4-alkyl is preferred.

As C1-C10-alkyl of R²⁰⁶, C1-C4-alkyl is preferred.

As C1-C10-alkyl of R²⁰⁷, C1-C4-alkyl is preferred.

As C1-C10-alkyl of R²⁰⁸, C1-C4-alkyl is preferred.

As C1-C10-alkyl of R²⁰⁹, C1-C4-alkyl is preferred.

As C1-C4-alkyl of R^{x}, methyl is preferred.

As C3-C10-alkyl of R⁷, C4-C8-alkyl is preferred.

As C1-C10-alkyl of —(C1-C10-alkylene)-NH—C(═O)—O—(C1-C10-alkyl) in R⁷, C1-C6-alkyl is preferred. More preferable C1-C10-alkyl of —(C1-C10-alkylene)-NH—C(═O)—O—(C1-C10-alkyl) in R⁷ is C3-C6-alkyl.

As C1-C10-alkyl of —(C1-C10-alkylene)-O—(C1-C10-alkyl) in R⁷, C1-C6-alkyl is preferred. More preferable C1-C10-alkyl of —(C1-C10-alkylene)-O—(C1-C10-alkyl) in R⁷ is C3-C6-alkyl.

As C1-C10-alkyl of R⁷¹, C1-C4-alkyl is preferred.

As C1-C10-alkyl of R⁷², C1-C4-alkyl is preferred.

As C1-C10-alkyl of substituents in R⁷³, C1-C4-alkyl is preferred.

As C1-C10-alkyl of R¹⁰¹, C1-C4-alkyl is preferred.

As C1-C10-alkyl of R¹⁰², C1-C4-alkyl is preferred.

As C1-C10-alkyl of R¹⁰³, C1-C4-alkyl is preferred.

As C1-C10-alkyl of R¹⁰⁴, C1-C4-alkyl is preferred.

As C1-C10-alkyl of R^{10′}, C1-C4-alkyl is preferred.

As C1-C10-alkyl of R¹⁰⁶, C1-C4-alkyl is preferred.

As C1-C10-alkyl of R¹⁰⁷, C1-C4-alkyl is preferred.

As C1-C10-alkyl of R^{10′}, C1-C4-alkyl is preferred.

As C1-C10-alkyl of R¹⁰⁹, C1-C4-alkyl is preferred.

As C1-C10-alkyl of R⁶, C1-C4-alkyl is preferred. More preferable C1-C10-alkyl of R⁶ is methyl.

As C1-C10-alkyl of R¹, C1-C4-alkyl is preferred. More preferable C1-C10-alkyl of R¹ is methyl.

As C1-C10-alkyl of R²¹ C1-C4-alkyl is preferred.

As C1-C10-alkyl of R², C1-C4-alkyl is preferred.

As C1-C10-alkyl of substituents in C3-C10-cycloalkyl of R², C1-C4-alkyl is preferred.

As C1-C10-alkyl of R¹, C1-C4-alkyl is preferred.

As C1-C10-alkyl of R⁹, C1-C4-alkyl is preferred.

As C1-C10-alkyl of R¹⁰, C1-C4-alkyl is preferred.

As C1-C10-alkyl of R¹¹, C1-C4-alkyl is preferred.

As C1-C10-alkyl of —C(═O)—(C1-C10-alkyl) in R⁸, C1-C4-alkyl is preferred.

As C1-C10-alkyl of —C(═O)—(C1-C10-alkyl) in R⁹, C1-C4-alkyl is preferred. More preferable C1-C10-alkyl of —C(═O)—(C1-C10-alkyl) in R⁹ is methyl.

As C1-C10-alkyl of —C(═O)—(C1-C10-alkyl) in R¹⁰, C1-C4-alkyl is preferred.

As C1-C10-alkyl of —C(═O)—(C1-C10-alkyl) in R¹¹, C1-C4-alkyl is preferred.

As C1-C10-alkyl of —C(═O)—O—(C1-C10-alkyl) in R⁸, C1-C4-alkyl is preferred.

As C1-C10-alkyl of —C(═O)—O—(C1-C10-alkyl) in R⁹, C1-C4-alkyl is preferred.

As C1-C10-alkyl of —C(═O)—O—(C1-C10-alkyl) in R¹⁰, C1-C4-alkyl is preferred.

As C1-C10-alkyl of —C(═O)—O—(C1-C10-alkyl) in R¹¹, C1-C4-alkyl is preferred.

As C1-C10-alkyl of —C(═O)—NR⁸¹—(C1-C10-alkyl) in R⁸, C1-C4-alkyl is preferred.

As C1-C10-alkyl of —C(═O)—NR⁸¹—(C1-C10-alkyl) in R⁹, C1-C4-alkyl is preferred.

As C1-C10-alkyl of —C(═O)—NR⁸¹—(C1-C10-alkyl) in R¹⁰, C1-C4-alkyl is preferred.

As C1-C10-alkyl of —C(═O)—NR⁸¹—(C1-C10-alkyl) in R¹¹, C1-C4-alkyl is preferred.

As C1-C10-alkyl of —NR⁸⁴—C(═O)—(C1-C10 alkyl) in R⁸, C1-C4-alkyl is preferred.

More preferable C1-C10-alkyl of —NR⁸⁴—C(═O)—(C1-C10 alkyl) in R⁸ is methyl.

As C1-C10-alkyl of —NR⁸⁴—C(═O)—(C1-C10 alkyl) in R⁹, C1-C4-alkyl is preferred.

As C1-C10-alkyl of —NR⁸⁴—C(═O)—(C1-C10 alkyl) in R¹⁰, C1-C4-alkyl is preferred.

As C1-C10-alkyl of —NR⁸⁴—C(═O)—(C1-C10 alkyl) in R¹¹, C1-C4-alkyl is preferred.

As C1-C10-alkyl of —NR⁸⁷—C(═O)—O—(C1-C10-alkyl) in R⁸, C1-C4-alkyl is preferred.

As C1-C10-alkyl of —NR⁸⁷—C(═O)—O—(C1-C10-alkyl) in R⁹, C1-C4-alkyl is preferred.

As C1-C10-alkyl of —NR⁸⁷—C(═O)—O—(C1-C10-alkyl) in R¹⁰, C1-C4-alkyl is preferred.

As C1-C10-alkyl of —NR⁸⁷—C(═O)—O—(C1-C10-alkyl) in R¹¹, C1-C4-alkyl is preferred.

As C1-C10-alkyl of —SO₂—(C1-C10-alkyl) in R⁸, C1-C4-alkyl is preferred.

As C1-C10-alkyl of —SO₂—(C1-C10-alkyl) in R⁹, C1-C4-alkyl is preferred.

As C1-C10-alkyl of —SO₂—(C1-C10-alkyl) in R¹⁰, C1-C4-alkyl is preferred.

As C1-C10-alkyl of —SO₂—(C1-C10-alkyl) in R¹¹, C1-C4-alkyl is preferred.

As C1-C10-alkyl of substituents in R⁸, C1-C4-alkyl is preferred.

As C1-C10-alkyl of substituents in R⁹, C1-C4-alkyl is preferred.

As C1-C10-alkyl of substituents in R¹⁰, C1-C4-alkyl is preferred.

As C1-C10-alkyl of substituents in R¹¹, C1-C4-alkyl is preferred.

As C1-C4-alkyl of R⁸¹, methyl is preferred.

As C1-C4-alkyl of R⁸², methyl is preferred.

As C1-C4-alkyl of R⁸³, methyl is preferred.

As C1-C4-alkyl of R⁸⁴, methyl is preferred.

As C1-C4-alkyl of R⁸⁵, methyl is preferred.

As C1-C4-alkyl of R⁸⁶, methyl is preferred.

As C1-C4-alkyl of R⁸⁷, methyl is preferred.

As C1-C4-alkyl of R⁸⁸, methyl is preferred.

As C1-C4-alkyl of R⁸⁹, methyl is preferred.

As C1-C10-alkyl of R^{1a}, C1-C4-alkyl is preferred. More preferable C1-C10-alkyl of R^{1a} is methyl.

As C1-C10-alkyl of R^{5a}, C1-C4-alkyl is preferred. More preferable C1-C10-alkyl of R^{5a} is methyl.

As C1-C10-alkyl of R^{1b}, C1-C4-alkyl is preferred. More preferable C1-C10-alkyl of R^{1b} is methyl.

As C1-C10-alkyl of R^{1c}, C1-C4-alkyl is preferred. More preferable C1-C10-alkyl of R^{1c} is methyl.

As C1-C10-alkyl of R¹³, C1-C4-alkyl is preferred.

As C1-C10-alkyl of R¹⁴, C1-C4-alkyl is preferred.

As C1-C10-alkyl of R¹⁵, C1-C4-alkyl is preferred.

As C1-C10-alkyl of —C(═O)—(C1-C10-alkyl) in R¹³, C1-C4-alkyl is preferred.

As C1-C10-alkyl of —C(═O)—(C1-C10-alkyl) in R¹⁴, C1-C4-alkyl is preferred.

As C1-C10-alkyl of —C(═O)—(C1-C10-alkyl) in R¹⁵, C1-C4-alkyl is preferred.

The term “alkoxy,” as used herein, refers to an alkyl group, as defined herein, appended to the parent molecular moiety through an oxygen atom. Representative examples of alkoxy include, but are not limited to, methoxy, ethoxy, propoxy, 2-propoxy, butoxy and tert-butoxy.

As C1-C10-alkoxy of R⁴, C1-C4-alkoxy is preferred.

As C1-C10-alkoxy of R⁵, C1-C4-alkoxy is preferred.

As C1-C10-alkoxy of R⁶, C1-C4-alkoxy is preferred.

As C1-C10-alkoxy of R¹, C1-C4-alkoxy is preferred.

As C1-C10-alkoxy of R²¹, C1-C4-alkoxy is preferred.

As C1-C10-alkoxy substituents in R⁸, C1-C4-alkoxy is preferred.

As C1-C10-alkoxy substituents in R⁹, C1-C4-alkoxy is preferred.

As C1-C10-alkoxy substituents in R¹⁰, C1-C4-alkoxy is preferred.

As C1-C10-alkoxy substituents in R¹¹, C1-C4-alkoxy is preferred.

The term “alkenyl,” as used herein, means a straight or branched, hydrocarbon chain containing at least one carbon-carbon double bond and from 2 to 10 carbon atoms.

As C2-C10-alkenyl of R⁴, C2-C4-alkenyl is preferred.

As C2-C10-alkenyl of R⁵, C2-C4-alkenyl is preferred.

As C2-C10-alkenyl of R⁶, C2-C4-alkenyl is preferred.

The term “alkenylene” as used herein, refers to a divalent group derived from a straight or branched hydrocarbon chain containing at least one carbon-carbon double bond and from 2 to 10 carbon atoms.

As C2-C4-alkenylene of L, ethenylene is preferred.

The term “alkynyl,” as used herein, means a straight or branched, hydrocarbon chain containing at least one carbon-carbon triple bond and from 2 to 10 carbon atoms.

As C2-C10-alkynyl of R⁴, C2-C4-alkynyl is preferred.

As C2-C10-alkynyl of R⁵, C2-C4-alkynyl is preferred.

As C2-C10-alkynyl of R⁶, C2-C4-alkynyl is preferred.

The term “alkoxyalkyl,” as used herein, refers to an alkoxy group, as defined herein, appended to the parent molecular moiety through an alkyl group, as defined herein.

The term “alkoxyfluoroalkyl,” as used herein, refers to an alkoxy group, as defined herein, appended to the parent molecular moiety through a fluoroalkyl group, as defined herein.

The term “alkylene,” as used herein, refers to a divalent group derived from a straight or branched chain hydrocarbon of 1 to 10 carbon atoms, for example, of 2 to 5 carbon atoms. Representative examples of alkylene include, but are not limited to, —CH₂—, —CH₂CH₂—, —CH₂CH₂CH₂—, —CH₂CH₂CH₂CH₂—, —C(CH₃)₂—CH₂—, —CH₂CH₂CH₂CH₂CH₂—, —CH₂—C(CH₃)₂—, —CH(CH₃)—CH₂—, —CH₂—CH(CH₃)— and —CH(CH₃)—.

As C1-C10-alkylene of —(C1-C10-alkylene)-O— in L, —C1-C4-alkylene is preferred. More preferable C1-C10-alkylene of —(C1-C10-alkylene)-O— in L is —CH₂—.

As C1-C10-alkylene of —O—(C1-C10-alkylene)- in L, —C1-C4-alkylene is preferred. More preferable C1-C10-alkylene of —O—(C1-C10-alkylene)- in L is —CH₂—.

As C2-C10-alkylene of C2-C10-alkylene-cyano in R⁷, C2-C6-alkylene is preferred. More preferable C2-C10-alkylene of —(C2-C10-alkylene)-cyano in R⁷ is C2-C4-alkylene.

As C1-C10-alkylene of —(C1-C10-alkylene)-NH—C(═O)—O—(C1-C10-alkyl) in R⁷, C1-C6-alkylene is preferred. More preferable C1-C10-alkylene of —(C1-C10-alkylene)-NH—C(═O)—O—(C1-C10-alkyl) in R⁷ is C3-C6-alkylene.

As C1-C10-alkylene of —(C1-10-alkylene)-O—(C1-C10-alkyl) in R⁷, C1-C6-alkylene is preferred. More preferable C1-C10-alkylene of —(C1-10-alkylene)-O—(C1-C10-alkyl) in R⁷ is C3-C6-alkylene.

As C1-C10-alkylene of —(C1-C10-alkylene)-(CR⁷¹R⁷²)ₚ—R⁷³ in R⁷, C1-C6-alkylene is preferred. More preferable C1-C10-alkylene of —(C1-C10-alkylene)-(CR⁷¹R⁷²)ₚ—R⁷³ in R⁷ is C3-C6-alkylene.

As C1-C10-alkylene of M, C1-C4-alkylene is preferred. More preferable C1-C10-alkylene of M is C1-C2-alkylene.

As C1-C10-alkylene of M¹, C1-C4-alkylene is preferred. More preferable C1-C10-alkylene of M¹ is C1-C2-alkylene.

As C1-C4-alkylene of M^{1z}, C1-2-alkylene is preferred. More preferable C1-C4-alkylene of M^{1z} is methylene or ethylene.

As C1-C10-alkylene of M^{a}, C1-C6-alkylene is preferred. More preferable C1-C4-alkylene of M^{a} is C1-C2-alkylene. More preferable C1-C10-alkylene of M^{a} is methylene or ethylene.

The term “alkynylene” as used herein, refers to a divalent group derived from a straight or branched hydrocarbon chain containing at least one carbon-carbon triple bond and from 2 to 10 carbon atoms.

As C2-C4-alkynylene of L, ethynylene is preferred.

The term “alkylamino,” as used herein, means at least one alkyl group, as defined herein, is appended to the parent molecular moiety through an amino group, as defined herein.

The term “amide,” as used herein, means —C(O)NR^{E}— or —NR^{E}C(O)—, wherein R^{E} may be hydrogen, alkyl, cycloalkyl, aryl, heteroaryl, heterocycle, alkenyl, or heteroalkyl.

The term “aminoalkyl,” as used herein, means at least one amino group, as defined herein, is appended to the parent molecular moiety through an alkylene group, as defined herein.

The term “amino,” as used herein, means —NR^{F}R^{G}, wherein R^{F} and R^{G} may be hydrogen, alkyl, cycloalkyl, aryl, heteroaryl, heterocycle, alkenyl, or heteroalkyl. In the case of an aminoalkyl group or any other moiety where amino appends together two other moieties, amino may be —NR^{H}—, wherein R^{H} may be hydrogen, alkyl, cycloalkyl, aryl, heteroaryl, heterocycle, alkenyl, or heteroalkyl.

The term “aryl,” as used herein, refers to a phenyl group, or a bicyclic fused ring system. Bicyclic fused ring systems are exemplified by a phenyl group appended to the parent molecular moiety and fused to a cycloalkyl group, as defined herein, a phenyl group, a heteroaryl group, as defined herein, or a heterocycle, as defined herein. Such a bicyclic fused ring system is comprised of no more than fifteen atoms. The term “6 to 15 membered aryl” means the ring system comprising of 6 to 15 atoms. The term “6 to 10 membered aryl” means the ring system comprising of 6 to 10 atoms. Representative examples of aryl include, but are not limited to, indolyl, naphthyl, phenyl, tetrahydroquinolinyl, 2,3-dihydrobenzo[1,4]dioxine-6-yl, indazole-5-yl, and benzo[1,3]dioxole-5-yl, benzofuran-5-yl.

As -(6 to 15 membered aryl)- of L, 6 to 10 membered aryl is preferred.

As 6 to 15 membered aryl of R⁴, 6 to 10 membered aryl is preferred.

As 6 to 15 membered aryl of R⁵, 6 to 10 membered aryl is preferred.

As 6 to 15 membered aryl of Q, 6 to 10 membered aryl is preferred.

As 6 to 15 membered aryl of R, 6 to 10 membered aryl is preferred. More preferable 6 to 15 membered aryl of R is phenyl.

As 6 to 15 membered aryl of R⁷³, 6 to 10 membered aryl is preferred.

As 6 to 15 membered aryl of Ring B, 6 to 10 membered aryl is preferred.

As 6 to 15 membered aryl of Ring D, 6 to 10 membered aryl is preferred. More preferable Ring D is phenyl.

The term “cyanoalkyl,” as used herein, means at least one —CN group, is appended to the parent molecular moiety through an alkylene group, as defined herein.

The term “cyanofluoroalkyl,” as used herein, means at least one —CN group, is appended to the parent molecular moiety through a fluoroalkyl group, as defined herein.

The term “cycloalkoxy,” as used herein, refers to a cycloalkyl group, as defined herein, appended to the parent molecular moiety through an oxygen atom.

The term “cycloalkyl,” as used herein, refers to a monocarbocyclic ring system or a bicarbocyclic ring system containing three to ten carbon atoms, zero heteroatoms and zero double bonds. Representative examples of cycloalkyl include, but are not limited to, cyclopropyl, cyclobutyl, cyclopentyl, cyclohexyl, cycloheptyl, cyclooctyl, cyclononyl and cyclodecyl. “Cycloalkyl” also includes carbocyclic ring systems in which a cycloalkyl group is appended to the parent molecular moiety and is fused to an aryl group as defined herein (e.g., a phenyl group), a heteroaryl group as defined herein, or a heterocycle as defined herein. Representative examples of cycloalkyl also include, but are not limited to, 4,5,6,7-tetrahydro-1H-indazolyl. The bicarbocyclic ring system is a monocarobocyclic ring system fused to a monocarboxylic ring system, a spiro cycloalkyl group or a bridged monocarboxylic ring system in which two non-adjacent atoms of the ring are linked by an alkylene bridge of 1, 2, 3, 4 carbon atoms. Representative example bicarbocyclic ring systems include, but are not limited, spiro[2.2]pentanyl, spiro[2.4]heptanyl, spiro[3.5]nonanyl, bicyclo[2.2.1]heptanyl or bicyclo[2.2.2]octanyl.

As C3-C10-cycloalkyl of R⁴, C3-C6-cycloalkyl is preferred.

As C3-C10-cycloalkyl of R⁵, C3-C6-cycloalkyl is preferred.

As C3-C10-cycloalkyl which is formed R^{c} and R^{d} together with the carbon atom to which they are attached, C3-C7-cycloalkyl is preferred.

As C3-C10-cycloalkyl of Q, C3-C8-cycloalkyl is preferred.

As C3-C10-cycloalkyl of R², C3-C6-cycloalkyl is preferred.

As C3-C10-cycloalkyl which is formed R⁷¹ and R⁷² together with the carbon atom to which they are attached, C3-C7-cycloalkyl is preferred.

As C3-C10-cycloalkyl of R⁷³, C3-C8-cycloalkyl is preferred.

As C3-C10-cycloalkyl of Ring B, C3-C8-cycloalkyl is preferred.

As C3-C10-cycloalkyl of —C(═O)—(C3-C10-cycloalkyl) in R¹, C3-C7-cycloalkyl is preferred. More preferable C3-C10-cycloalkyl of —C(═O)—(C3-C10-cycloalkyl) in R¹ is cyclopropyl, cyclobutyl, cyclopentyl, cyclohexyl or cycloheptyl. Most preferable C3-C10-cycloalkyl of —C(═O)—(C3-C10-cycloalkyl) in R⁸ is cyclopropyl.

As C3-C10-cycloalkyl of —C(═O)—(C3-C10-cycloalkyl) in R⁹, C3-C7-cycloalkyl is preferred. More preferable C3-C10-cycloalkyl of —C(═O)—(C3-C10-cycloalkyl) in R⁹ is cyclopropyl, cyclobutyl, cyclopentyl, cyclohexyl or cycloheptyl. Most preferable C3-C10-cycloalkyl of —C(═O)—(C3-C10-cycloalkyl) in R⁹ is cyclopropyl.

As C3-C10-cycloalkyl of —C(═O)—(C3-C10-cycloalkyl) in R¹⁰, C3-C7-cycloalkyl is preferred. More preferable C3-C10-cycloalkyl of —C(═O)—(C3-C10-cycloalkyl) in R¹⁰ is cyclopropyl, cyclobutyl, cyclopentyl, cyclohexyl or cycloheptyl. Most preferable C3-C10-cycloalkyl of —C(═O)—(C3-C10-cycloalkyl) in R¹⁰ is cyclopropyl.

As C3-C10-cycloalkyl of —C(═O)—(C3-C10-cycloalkyl) in R¹¹, C3-C7-cycloalkyl is preferred. More preferable C3-C10-cycloalkyl of —C(═O)—(C3-C10-cycloalkyl) in R¹⁰ is cyclopropyl, cyclobutyl, cyclopentyl, cyclohexyl or cycloheptyl. Most preferable C3-C10-cycloalkyl of —C(═O)—(C3-C10-cycloalkyl) in R¹¹ is cyclopropyl.

As C3-C10-cycloalkyl of —C(═O)—O—(C3-C10-cycloalkyl) in R⁸, C3-C7-cycloalkyl is preferred.

As C3-C10-cycloalkyl of —C(═O)—O—(C3-C10-cycloalkyl) in R⁹, C3-C7-cycloalkyl is preferred.

As C3-C10-cycloalkyl of —C(═O)—O—(C3-C10-cycloalkyl) in R¹⁰, C3-C7-cycloalkyl is preferred.

As C3-C10-cycloalkyl of —C(═O)—O—(C3-C10-cycloalkyl) in R¹¹, C3-C7-cycloalkyl is preferred.

As C3-C10-cycloalkyl of —C(═O)—NR⁸²—(C3-C10-cycloalkyl) in R⁹, C3-C7-cycloalkyl is preferred.

As C3-C10-cycloalkyl of —C(═O)—NR⁸²—(C3-C10-cycloalkyl) in R⁹, C3-C7-cycloalkyl is preferred.

As C3-C10-cycloalkyl of —C(═O)—NR⁸²—(C3-C10-cycloalkyl) in R¹⁰, C3-C7-cycloalkyl is preferred.

As C3-C10-cycloalkyl of —C(═O)—NR⁸²—(C3-C10-cycloalkyl) in R¹¹, C3-C7-cycloalkyl is preferred.

As C3-C10-cycloalkyl of —NR⁸⁵—C(═O)—(C3-C10-cycloalkyl) in R⁸, C3-C7-cycloalkyl is preferred.

As C3-C10-cycloalkyl of —NR⁸⁵—C(═O)—(C3-C10-cycloalkyl) in R⁹, C3-C7-cycloalkyl is preferred.

As C3-C10-cycloalkyl of —NR⁸⁵—C(═O)—(C3-C10-cycloalkyl) in R¹⁰, C3-C7-cycloalkyl is preferred.

As C3-C10-cycloalkyl of —NR⁸⁵—C(═O)—(C3-C10-cycloalkyl) in R¹¹, C3-C7-cycloalkyl is preferred.

As C3-C10-cycloalkyl of —NR⁸⁸—C(═O)—O—(C3-C10-cycloalkyl) in R⁸, C3-C7-cycloalkyl is preferred.

As C3-C10-cycloalkyl of —NR⁸⁸—C(═O)—O—(C3-C10-cycloalkyl) in R⁹, C3-C7-cycloalkyl is preferred.

As C3-C10-cycloalkyl of —NR⁸⁸—C(═O)—O—(C3-C10-cycloalkyl) in R¹⁰. C3-C7-cycloalkyl is preferred.

As C3-C10-cycloalkyl of —NR⁸⁸—C(═O)—O—(C3-C10-cycloalkyl) in R¹¹, C3-C7-cycloalkyl is preferred.

As C3-C10-cycloalkyl of Ring C, C3-C8-cycloalkyl is preferred.

As C3-C10-cycloalkyl of —C(═O)—(C3-C10-cycloalkyl) in R¹³, C3-C7-cycloalkyl is preferred.

As C3-C10-cycloalkyl of —C(═O)—(C3-C10-cycloalkyl) in R¹⁴, C3-C7-cycloalkyl is preferred.

As C3-C10-cycloalkyl of —C(═O)—(C3-C10-cycloalkyl) in R¹⁵, C3-C7-cycloalkyl is preferred.

As C3-C10-cycloalkyl of Ring B^{a}, C3-C8-cycloalkyl is preferred.

The term “cycloalkane,” as used herein, refers to a monocarbocyclic ring system or a bicarbocyclic ring system containing three to ten carbon atoms, zero heteroatoms and zero double bonds. Representative examples of cycloalkane include, but are not limited to, cyclopropane, cyclobutane, cyclopentane, cyclohexane, cycloheptane, cyclooctane, cyclononane and cyclodecane. “Cycloalkane” also includes carbocyclic ring systems in which a cycloalkane group is appended to the parent molecular moiety and is fused to an aryl group as defined herein (e.g., a phenyl group), a heteroaryl group as defined herein, or a heterocycle as defined herein. Representative examples of cycloalkane also include, but are not limited to, 4,5,6,7-tetrahydro-1H-indazole. The bicarbocyclic ring system is a monocarobocyclic ring system fused to a monocarbocyclic ring system, a spiro cycloalkane group or a bridged monocarbocyclic ring system in which two nonadjacent atoms of the ring are linked by an alkylene bridge of 1, 2, 3, 4 carbon atoms. Representative example bicarbocyclic ring systems include, but are not limited, spiro[2.2]pentane, spiro[2.4]heptane, spiro[3.5]nonane, bicyclo[2.2.1]heptane or bicyclo[2.2.2]octane.

As C3-C10-cycloalkane of —(C3-C10-cycloalkane)- in L, C3-C6-cycloalkane is preferred.

The term “cycloalkenyl,” as used herein, means a non-aromatic monocyclic or multicyclic ring system containing at least one carbon-carbon double bond and preferably having from 5-10 carbon atoms per ring. Exemplary monocyclic cycloalkenyl rings include cyclopentenyl, cyclohexenyl or cycloheptenyl.

The term “fluoroalkyl,” as used herein, means an alkyl group, as defined herein, in which one, two, three, four, five, six, seven or eight hydrogen atoms are replaced by fluorine. Representative examples of fluoroalkyl include, but are not limited to, 2-fluoroethyl, 2,2,2-trifluoroethyl, trifluoromethyl, difluoromethyl, pentafluoroethyl, and trifluoropropyl such as 3,3,3-trifluoropropyl.

The term “fluoroalkoxy,” as used herein, means at least one fluoroalkyl group, as defined herein, is appended to the parent molecular moiety through an oxygen atom. Representative examples of fluoroalkoxy include, but are not limited to, difluoromethoxy, trifluoromethoxy and 2,2,2-trifluoroethoxy.

The term “halogen” or “halo,” as used herein, means C1, Br, I, or F.

The term “haloalkyl,” as used herein, means an alkyl group, as defined herein, in which one, two, three, four, five, six, seven or eight hydrogen atoms are replaced by a halogen.

As C1-C10-haloalkyl of substituents in R⁴, C1-C4-haloalkyl is preferred.

As C1-C10-haloalkyl of substituents in R⁵, C1-C4-haloalkyl is preferred.

As C1-C10-haloalkyl of R^{b}, C1-C4-haloalkyl is preferred.

As C1-C10-haloalkyl of R, C1-C4-haloalkyl is preferred.

As C1-C10-haloalkyl of R^{d}, C1-C4-haloalkyl is preferred.

As C1-C10-haloalkyl of substituents in Q, C1-C4-haloalkyl is preferred.

As C1-C10-haloalkyl of R²⁰¹, C1-C4-haloalkyl is preferred.

As C1-C10-haloalkyl of R²⁰², C1-C4-haloalkyl is preferred.

As C1-C10-haloalkyl of R²⁰³, C1-C4-haloalkyl is preferred.

As C1-C10-haloalkyl of R²⁰⁴, C1-C4-haloalkyl is preferred.

As C1-C10-haloalkyl of R²⁰⁵, C1-C4-haloalkyl is preferred.

As C1-C10-haloalkyl of R²⁰⁶, C1-C4-haloalkyl is preferred.

As C1-C10-haloalkyl of R²⁰⁷, C1-C4-haloalkyl is preferred.

As C1-C10-haloalkyl of R²⁰⁸, C1-C4-haloalkyl is preferred.

As C1-C10-haloalkyl of R²⁰⁹, C1-C4-haloalkyl is preferred.

As C1-C4-haloalkyl of R^{x}, methyl which may be optionally substituted with 1 to 3 halogen is preferred.

As C1-C10-haloalkyl of R¹, C1-C4-haloalkyl is preferred.

As C1-C10-haloalkyl of R²¹, C1-C4-haloalkyl is preferred.

As C1-C10-haloalkyl of R², C1-C4-haloalkyl is preferred.

As C1-C10-haloalkyl of substituents in C3-C10-cycloalkyl of R², C1-C4-haloalkyl is preferred.

As C1-C10-haloalkyl of R⁷¹, C1-C4-haloalkyl is preferred.

As C1-C10-haloalkyl of R⁷², C1-C4-haloalkyl is preferred.

As C1-C10-haloalkyl of substituents in R⁷³, C1-C4-haloalkyl is preferred.

As C1-C10-haloalkyl of R¹⁰¹, C1-C4-haloalkyl is preferred.

As C1-C10-haloalkyl of R¹⁰², C1-C4-haloalkyl is preferred.

As C1-C10-haloalkyl of R¹⁰³, C1-C4-haloalkyl is preferred.

As C1-C10-haloalkyl of R¹⁰⁴, C1-C4-haloalkyl is preferred.

As C1-C10-haloalkyl of R¹⁰⁵, C1-C4-haloalkyl is preferred.

As C1-C10-haloalkyl of R¹⁰⁶. C1-C4-haloalkyl is preferred.

As C1-C10-haloalkyl of R¹⁰⁷, C1-C4-haloalkyl is preferred.

As C1-C10-haloalkyl of R^{10′}, C1-C4-haloalkyl is preferred.

As C1-C10-haloalkyl of R¹⁰⁹, C1-C4-haloalkyl is preferred.

As C1-C10-haloalkyl of substituents in R¹, C1-C4-haloalkyl is preferred.

As C1-C10-haloalkyl of substituents in R⁹, C1-C4-haloalkyl is preferred.

As C1-C10-haloalkyl of substituents in R¹⁰, C1-C4-haloalkyl is preferred.

As C1-C10-haloalkyl of substituents in R¹¹, C1-C4-haloalkyl is preferred.

As C1-C10-haloalkyl of R^{1c}, C1-C4-haloalkyl is preferred.

As C1-C10-haloalkyl of —C(═O)—(C1-C10-haloalkyl) in R¹³, C1-C4-haloalkyl is preferred.

As C1-C10-haloalkyl of —C(═O)—(C1-C10-haloalkyl) in R¹⁴, C1-C4-haloalkyl is preferred.

As C1-C10-haloalkyl of —C(═O)—C1-C10-haloalkyl in R¹⁵, C1-C4-haloalkyl is preferred.

The term “haloalkoxy,” as used herein, means at least one haloalkyl group, as defined herein, is appended to the parent molecular moiety through an oxygen atom.

As C1-C10-haloalkoxy of R¹, C1-C4-haloalkoxy is preferred.

As C1-C10-haloalkoxy of R²¹, C1-C4-haloalkoxy is preferred.

The term “halocycloalkyl,” as used herein, means a cycloalkyl group, as defined herein, in which one or more hydrogen atoms are replaced by a halogen.

The term “heteroalkyl,” as used herein, means an alkyl group, as defined herein, in which one or more of the carbon atoms has been replaced by a heteroatom selected from S, O, P and N. Representative examples of heteroalkyls include, but are not limited to, alkyl ethers, secondary and tertiary alkyl amines, amides, and alkyl sulfides.

As C2-C10-heteroalkyl of R⁴, C2-C4-heteroalkyl is preferred.

As C2-C10-heteroalkyl of R⁵, C2-C4-heteroalkyl is preferred.

The term “heteroaryl,” as used herein, refers to an aromatic monocyclic ring or an aromatic bicyclic ring system. The term “5 to 15 membered heteroaryl” means the ring system comprising of 5 to 15 atoms. The term “5 to 10 membered heteroaryl” means the ring system comprising of 5 to 10 atoms. The term “5 membered heteroaryl” means the ring system comprising of 5 atoms. The aromatic monocyclic rings are five or six membered rings containing at least one heteroatom independently selected from the group consisting of N, O and S (e.g. 1, 2, 3, or 4 heteroatoms independently selected from O, S, and N). The five membered aromatic monocyclic rings have two double bonds and the six membered six membered aromatic monocyclic rings have three double bonds. The bicyclic heteroaryl groups are exemplified by a monocyclic heteroaryl ring appended to the parent molecular moiety and fused to a monocyclic cycloalkyl group, as defined herein, a monocyclic aryl group, as defined herein, a monocyclic heteroaryl group, as defined herein, or a monocyclic heterocycle, as defined herein. Such an aromatic bicyclic ring system is comprised of no more than fifteen atoms. Representative examples of heteroaryl include, but are not limited to, indolyl, pyridinyl (including pyridin-2-yl, pyridin-3-yl, pyridin-4-yl), pyrimidinyl, pyrimidine-5-yl, pyrazinyl, pyridazinyl, pyrazolyl, pyrazole-1,3-yl, pyrazole-1,4-yl, pyrrolyl, benzopyrazolyl, 1,2,3-triazolyl, thiophenyl, thiophene-2-yl, 1,3,4-thiadiazolyl, 1,2,4-thiadiazolyl, 1,3,4-oxadiazolyl, 1,2,4-oxadiazolyl, imidazolyl, thiazolyl, isothiazolyl, triazolyl, thiazole-5-yl, tetrazolyl, thienyl, benzimidazolyl, benzothiazolyl, benzoxazolyl, benzoxadiazolyl, benzothienyl, benzofuranyl, isobenzofuranyl, furanyl, oxazolyl, isoxazolyl, purinyl, isoindolyl, quinoxalinyl, indazolyl, quinazolinyl, 1,2,4-triazinyl, 1,3,5-triazinyl, isoquinolinyl, quinolinyl, quinoline-3-yl, 6,7-dihydro-1,3-benzothiazolyl, imidazo[1,2-a]pyridinyl, naphthyridinyl, pyridoimidazolyl, thiazolo[5,4-b]pyridin-2-yl, thiazolo[5,4-d]pyrimidin-2-yl, [1,2,4]triazolo[1,5-a]pyridine, [1,2,4]triazolo[4,3-a]pyridine, [1,2,4]triazolo[1,5-a]pyridine and 4,5,6,7-tetrahydro-1H-indazolyl.

As -(5 to 15 membered heteroaryl)- of L, 5 to 10 membered heteroaryl is preferred.

More preferable (5 to 15 membered heteroaryl)- of L is 5-membered heteroaryl.

As 5 to 15 membered heteroaryl of R⁴, 5 to 10 membered heteroaryl is preferred.

As 5 to 15 membered heteroaryl of R⁵, 5 to 10 membered heteroaryl is preferred.

As 5 to 15 membered heteroaryl of Q, 5 to 10 membered heteroaryl is preferred.

As 5 to 15 membered heteroaryl of R, 5 to 10 membered heteroaryl is preferred. More preferable 5 to 15 membered heteroaryl of R is 5 membered heteroaryl. Most preferable 5 to 15 membered heteroaryl of R is thiophenyl.

As 5 to 15 membered heteroaryl of R⁷³, 5 to 10 membered heteroaryl is preferred.

As 5 to 15 membered heteroaryl of Ring B, 5 to 10 membered heteroaryl is preferred.

As 5 to 15 membered heteroaryl of Ring C, 5 to 10 membered heteroaryl is preferred.

As 5 to 15 membered heteroaryl of Ring D, 5 to 10 membered heteroaryl is preferred.

More preferable 5 to 15 membered heteroaryl of Ring D is 5 membered heteroaryl.

As 5 membered heteroaryl of Ring D¹, pyrazolyl or oxadiazolyl is preferred. More preferable 5 membered heteroaryl of Ring D¹ is

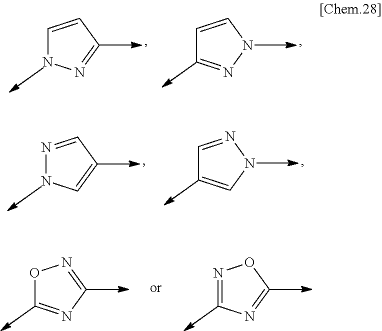 [SMILES: c1cn[nH]c1 ; c1cn[nH]c1 ; c1cn[nH]c1 ; c1cn[nH]c1 ; c1ncon1 ; c1ncon1]

wherein right arrow is connecting position with 6 membered ring and left arrow is connecting position with R^{Y}.

Most preferable 5 membered heteroaryl of Ring D¹ is

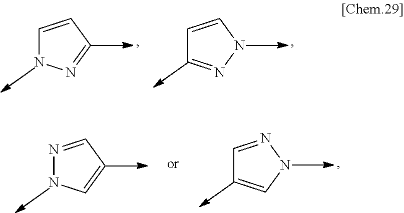 [SMILES: c1cn[nH]c1 ; c1cn[nH]c1 ; c1cn[nH]c1 ; c1cn[nH]c1]

wherein right arrow is connecting position with 6 membered ring and left arrow is connecting position with R^{Y}.

As 5 to 15 membered heteroaryl of Ring B^{a}, 5 to 10 membered heteroaryl is preferred.

The term “heterocycle” or “heterocyclic,” as used herein, means a monocyclic heterocycle, a bicyclic heterocycle, or a tricyclic heterocycle that is comprised of three to fifteen atoms. The term “3 to 15 membered heterocycle” means the ring system comprising of 3 to 15 atoms. The term “3 to 10 membered heterocycle” means the ring system comprising of 3 to 10 atoms. The term “4 to 6 membered heterocycle” means the ring system comprising of 4 to 6 atoms. The monocyclic heterocycle is a three-, four-, five-, six-, seven-, or eight-membered ring containing at least one heteroatom independently selected from the group consisting of O, N, and S. The three- or four-membered ring contains zero or one double bond, and one heteroatom selected from the group consisting of O, N, and S. The five-membered ring contains zero or one double bond and one, two or three heteroatoms selected from the group consisting of O, N and S. The six-membered ring contains zero, one or two double bonds and one, two, or three heteroatoms selected from the group consisting of O, N, and S. The seven- and eight-membered rings contains zero, one, two, or three double bonds and one, two, or three heteroatoms selected from the group consisting of O, N, and S. Representative examples of monocyclic heterocycles include, but are not limited to, azetidinyl, azetidine-3-yl, azepanyl, azepane-4-yl, aziridinyl, diazepanyl, 1,3-dioxanyl, 1,4-dioxane-2-yl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, morpholine-2-yl, 2-oxo-3-piperidinyl, 2-oxoazepan-3-yl, oxadiazolinyl, oxadiazolidinyl, oxazolinyl, oxazolidinyl, oxetanyl, oxepanyl, oxocanyl, piperazinyl, piperazine-1-yl, piperidinyl, piperidine-3-yl, piperidine-4-yl, pyranyl, pyrazolinyl, pyrazolidinyl, pyrrolinyl, pyrrolidine-3-yl, pyrrolidinyl, tetrahydrofuranyl, tetrahydrofuran-2-yl, tetrahydrofuran-3-yl, tetrahydropyranyl, tetrahydropyran-2-yl, tetrahydropyran-3-yl, tetrahydropyridinyl, tetrahydrothienyl, thiadiazolinyl, thiadiazolidinyl, 1,2-thiazinanyl, 1,3-thiazinanyl, thiazolinyl, thiazolidinyl, thiomorpholinyl, 1,1-dioxidothiomorpholinyl (thiomorpholine sulfone), thiopyranyl, trithianyl, 3-azabicyclo[3.1.1]heptanyl, and 7-oxabicyclo[2.2.1]heptanyl. The bicyclic heterocycle is a monocyclic heterocycle fused to a phenyl group, or a monocyclic heterocycle fused to a monocyclic cycloalkyl, or a monocyclic heterocycle fused to a monocyclic cycloalkenyl, or a monocyclic heterocycle fused to a monocyclic heterocycle, or a spiro heterocycle group, or a bridged monocyclic heterocycle ring system in which two non-adjacent atoms of the ring are linked by an alkylene bridge of 1, 2, 3, or 4 carbon atoms, or an alkenylene bridge of 2, 3, or 4 carbon atoms, an alkoxy bridge of 1, 2, 3, or 4 carbon atoms and 1, 2 oxygen atoms. Representative examples of bicyclic heterocycles include, but are not limited to, benzopyranyl, benzothiopyranyl, chromanyl, 2,3-dihydrobenzofuranyl, 2,3-dihydrobenzothienyl, 2,3-dihydroisoquinoline, 2-azaspiro[3.3]heptan-2-yl, 2-azaspiro[3.3]heptane-6-yl, 2-oxa-6-azaspiro[3.3]heptan-6-yl, azabicyclo[2.2.1]heptyl (including 2-azabicyclo[2.2.1]hept-2-yl), azabicyclo[3.1.0]hexanyl (including 3-azabicyclo[3.1.0]hexan-3-yl), 2,3-dihydro-1H-indolyl, 6-azaspiro[3.4]octane-2-yl, 7-azaspiro[3.5]nonane-2-yl, isoindolinyl, octahydrocyclopenta[c]pyrrolyl, octahydrocyclopenta[c]pyrrole-5-yl, octahydropyrrolopyridinyl, tetrahydroisoquinolinyl, 1,2,4]triazolo[1,5-a]pyridine, [1,2,4]triazolo[4,3-a]pyridine, [1,2,4]triazolo[1,5-a]pyridine and oxabicyclo[2.2.1]heptane-2-yl, 3-azabicyclo[3.1.1]heptane-6-yl.

Tricyclic heterocycles are exemplified by a bicyclic heterocycle fused to a phenyl group, or a bicyclic heterocycle fused to a monocyclic cycloalkyl, or a bicyclic heterocycle fused to a monocyclic cycloalkenyl, or a bicyclic heterocycle fused to a monocyclic heterocycle, or a bicyclic heterocycle in which two non-adjacent atoms of the bicyclic ring are linked by an alkylene bridge of 1, 2, 3, or 4 carbon atoms, or an alkenylene bridge of two, three, or four carbon atoms. Examples of tricyclic heterocycles include, but are not limited to, octahydro-2,5-epoxypentalene, hexahydro-2H-2,5-methanocyclopenta[b]furan, hexahydro-1H-1,4-methanocyclopenta[c]furan, aza-adamantane (1-azatricyclo[3.3.1.13,7]decane), and oxa-adamantane (2-oxatricyclo[3.3.1.13,7]decane). The monocyclic, bicyclic, and tricyclic heterocycles are connected to the parent molecular moiety through any carbon atom or any nitrogen atom contained within the rings, and can be unsubstituted or substituted.

As 3 to 15 membered heterocycle of -(3 to 15 membered heterocycle)- in L, 3 to 10 membered heterocycle is preferred. More preferable 3 to 15 membered heterocycle of -(3 to 15 membered heterocycle)- in L is 4 to 6 membered heterocycle.

As 3 to 15 membered heterocycle of R⁴, 4 to 6 membered heterocycle is preferred.

As 3 to 15 membered heterocycle of R⁵, 4 to 6 membered heterocycle is preferred.

As 3 to 15 membered heterocycle of Q, 4 to 6 membered heterocycle is preferred.

As 3 to 15 membered heterocycle of R⁷³, 4 to 6 membered heterocycle is preferred.

As 3 to 15 membered heterocycle of Ring B, 3 to 10 membered heterocycle is preferred. More preferable 3 to 15 membered heterocycle of Ring B is 4 to 7 membered heterocycle. Furthermore preferable 3 to 15 membered heterocycle of Ring B is azetidine, pyrrolidine, piperidine, piperazine, azepane, 1, 4-diazepane, morpholine, tetrahydrofuran, tetrahydropyran, 1,4-dioxane, 1,4-diazepane, homomorpholine, 7-oxabicyclo[2.2.1]heptane or 3-azabicyclo[3.1.1]heptane. Most preferable 3 to 15 membered heterocycle of Ring B is azetidine, pyrrolidine, piperidine, piperazine, azepane, morpholine, tetrahydrofuran, tetrahydropyran, 1,4-dioxane. Especially most preferable 3 to 15 membered heterocycle of Ring B is azetidine, pyrrolidine, piperidine.

As 3 to 15 membered heterocycle of —C(═O)—(3 to 15 membered heterocycle) in R⁸, 3 to 10 membered heterocycle is preferred. More preferable 3 to 15 membered heterocycle of —C(═O)—(3 to 15 membered heterocycle) in R¹ is 4 to 6 membered heterocycle.

As 3 to 15 membered heterocycle of —C(═O)—(3 to 15 membered heterocycle) in R⁹, 3 to 10 membered heterocycle is preferred. More preferable 3 to 15 membered heterocycle of —C(═O)—(3 to 15 membered heterocycle) in R⁹ is 4 to 6 membered heterocycle.

As 3 to 15 membered heterocycle of —C(═O)—(3 to 15 membered heterocycle) in R¹⁰, 3 to 10 membered heterocycle is preferred. More preferable 3 to 15 membered heterocycle of —C(═O)—(3 to 15 membered heterocycle) in R¹⁰ is 4 to 6 membered heterocycle.

As 3 to 15 membered heterocycle of —C(═O)—(3 to 15 membered heterocycle) in R¹¹, 3 to 10 membered heterocycle is preferred. More preferable 3 to 15 membered heterocycle of —C(═O)—(3 to 15 membered heterocycle) in R¹⁰ is 4 to 6 membered heterocycle.

As 3 to 15 membered heterocycle of —C(═O)—O-(3 to 15 membered heterocycle) in R¹, 3 to 10 membered heterocycle is preferred. More preferable 3 to 15 membered heterocycle of —C(═O)—O-(3 to 15 membered heterocycle) in R⁸ is 4 to 6 membered heterocycle.

As 3 to 15 membered heterocycle of —C(═O)—O-(3 to 15 membered heterocycle) in R⁹, 3 to 10 membered heterocycle is preferred. More preferable 3 to 15 membered heterocycle of —C(═O)—O-(3 to 15 membered heterocycle) in R⁹ is 4 to 6 membered heterocycle.

As 3 to 15 membered heterocycle of —C(═O)—O-(3 to 15 membered heterocycle) in R¹⁰ 3 to 10 membered heterocycle is preferred. More preferable 3 to 15 membered heterocycle of —C(═O)—O-(3 to 15 membered heterocycle) in R¹⁰ is 4 to 6 membered heterocycle.

As 3 to 15 membered heterocycle of —C(═O)—O-(3 to 15 membered heterocycle) in R, 3 to 10 membered heterocycle is preferred. More preferable 3 to 15 membered heterocycle of —C(═O)—O-(3 to 15 membered heterocycle) in R¹¹ is 4 to 6 membered heterocycle.

As 3 to 15 membered heterocycle of —C(═O)—NR⁸³-(3 to 15 membered heterocycle) in R⁸, 3 to 10 membered heterocycle is preferred. More preferable 3 to 15 membered heterocycle of —C(═O)—NR⁸³-(3 to 15 membered heterocycle) in R⁸ is 4 to 6 membered heterocycle.

As 3 to 15 membered heterocycle of —C(═O)—NR⁸³-(3 to 15 membered heterocycle) in R⁹, 3 to 10 membered heterocycle is preferred. More preferable 3 to 15 membered heterocycle of —C(═O)—NR⁸³-(3 to 15 membered heterocycle) in R⁹ is 4 to 6 membered heterocycle.

As 3 to 15 membered heterocycle of —C(═O)—NR⁸³-(3 to 15 membered heterocycle) in R¹⁰, 3 to 10 membered heterocycle is preferred. More preferable 3 to 15 membered heterocycle of —C(═O)—NR⁸³-(3 to 15 membered heterocycle) in R¹⁰ is 4 to 6 membered heterocycle.

As 3 to 15 membered heterocycle of —C(═O)—NR⁸³-(3 to 15 membered heterocycle) in R¹¹, 3 to 10 membered heterocycle is preferred. More preferable 3 to 15 membered heterocycle of —C(═O)—NR⁸³-(3 to 15 membered heterocycle) in R¹¹ is 4 to 6 membered heterocycle.

As 3 to 15 membered heterocycle of —NR⁸⁶—C(═O)—(3 to 15 membered heterocycle) in R⁸, 3 to 10 membered heterocycle is preferred. More preferable 3 to 15 membered heterocycle of —NR⁸⁶—C(═O)—(3 to 15 membered heterocycle) in R⁸ is 4 to 6 membered heterocycle.

As 3 to 15 membered heterocycle of —NR⁸⁶—C(═O)—(3 to 15 membered heterocycle) in R⁹, 3 to 10 membered heterocycle is preferred. More preferable 3 to 15 membered heterocycle of —NR⁸⁶—C(═O)—(3 to 15 membered heterocycle) in R⁹ is 4 to 6 membered heterocycle.

As 3 to 15 membered heterocycle of —NR⁸⁶—C(═O)—(3 to 15 membered heterocycle) in R¹⁰, 3 to 10 membered heterocycle is preferred. More preferable 3 to 15 membered heterocycle of —NR⁸⁶—C(═O)—(3 to 15 membered heterocycle) in R¹⁰ is 4 to 6 membered heterocycle.

As 3 to 15 membered heterocycle of —NR⁸⁶—C(═O)—(3 to 15 membered heterocycle) in R¹¹, 3 to 10 membered heterocycle is preferred. More preferable 3 to 15 membered heterocycle of —NR⁸⁶—C(═O)—(3 to 15 membered heterocycle) in R¹⁰ is 4 to 6 membered heterocycle.

As 3 to 15 membered heterocycle of —NR⁸³—C(═O)—(3 to 15 membered heterocycle) in R¹¹, 3 to 10 membered heterocycle is preferred. More preferable 3 to 15 membered heterocycle of —NR⁸³—C(═O)—(3 to 15 membered heterocycle) in R¹¹ is 4 to 6 membered heterocycle.

As 3 to 15 membered heterocycle of —NR⁸⁹—C(═O)—O-(3 to 15 membered heterocycle) in R⁸, 3 to 10 membered heterocycle is preferred. More preferable 3 to 15 membered heterocycle of —NR⁸⁹—C(═O)—O-(3 to 15 membered heterocycle) in R⁸ is 4 to 6 membered heterocycle.

As 3 to 15 membered heterocycle of —NR⁸⁹—C(═O)—O-(3 to 15 membered heterocycle) in R⁹, 3 to 10 membered heterocycle is preferred. More preferable 3 to 15 membered heterocycle of —NR⁸⁹—C(═O)—O-(3 to 15 membered heterocycle) in R⁹ is 4 to 6 membered heterocycle.

As 3 to 15 membered heterocycle of —NR⁸⁹—C(═O)—O-(3 to 15 membered heterocycle) in R¹⁰, 3 to 10 membered heterocycle is preferred. More preferable 3 to 15 membered heterocycle of —NR⁸⁹—C(═O)—O-(3 to 15 membered heterocycle) in R¹⁰ is 4 to 6 membered heterocycle.

As 3 to 15 membered heterocycle of —NR⁸⁹—C(═O)—O-(3 to 15 membered heterocycle) in R¹¹, 3 to 10 membered heterocycle is preferred. More preferable 3 to 15 membered heterocycle of —NR⁸⁹—C(═O)—O-(3 to 15 membered heterocycle) in R¹¹ is 4 to 6 membered heterocycle.

As 3 to 15 membered heterocycle of —C(═O)—(3 to 15 membered heterocycle) in R¹³, 3 to 10 membered heterocycle is preferred. More preferable 3 to 15 membered heterocycle of —C(═O)—(3 to 15 membered heterocycle) in R¹³ is 4 to 6 membered heterocycle.

As 3 to 15 membered heterocycle of —C(═O)—(3 to 15 membered heterocycle) in R¹⁴, 3 to 10 membered heterocycle is preferred. More preferable 3 to 15 membered heterocycle of —C(═O)—(3 to 15 membered heterocycle) in R¹⁴ is 4 to 6 membered heterocycle.

As 3 to 15 membered heterocycle of —C(═O)—(3 to 15 membered heterocycle) in R¹⁵, 3 to 10 membered heterocycle is preferred. More preferable 3 to 15 membered heterocycle of —C(═O)—(3 to 15 membered heterocycle) in R¹⁵ is 4 to 6 membered heterocycle.

As 3 to 15 membered heterocycle of Ring B^{a}, 3 to 10 membered heterocycle is preferred. More preferable 3 to 15 membered heterocycle of Ring B^{a} is 4 to 7 membered heterocycle. Furthermore preferable 3 to 15 membered heterocycle of Ring B^{a} is azetidine, pyrrolidine, piperidine, piperazine, azepane, 1, 4-diazepane, morpholine, tetrahydrofuran, tetrahydropyran, 1,4-dioxane, 1,4-diazepane, homomorpholine, 7-oxabicyclo[2.2.1]heptane or 3-azabicyclo[3.1.1]heptane. Most preferable 3 to 15 membered heterocycle of Ring B^{a} is azetidine, pyrrolidine, piperidine, piperazine, azepane, morpholine, tetrahydrofuran, tetrahydropyran, 1,4-dioxane. Especially most preferable 3 to 15 membered heterocycle of Ring B^{a} is azetidine, pyrrolidine, piperidine.

The term “hydroxyl” or “hydroxy,” as used herein, means an —OH group.

The term “hydroxyalkyl,” as used herein, means at least one —OH group, is appended to the parent molecular moiety through an alkylene group, as defined herein.

The term “hydroxyfluoroalkyl,” as used herein, means at least one —OH group, is appended to the parent molecular moiety through a fluoroalkyl group, as defined herein.

The term “Pentahalosulfanyl”, as used herein, includes, but is not limited, SFS.

The term “thioalkyl”, as used herein, means a alkyl group, as defined herein, is appended to the parent moiety through a sulfur atom.

As C1-C10-thioalkyl of R⁴, C1-C4-thioalkyl is preferred.

As C1-C10-thioalkyl of R⁵, C1-C4-thioalkyl is preferred.

As C1-C10-thioalkyl of R⁶, C1-C4-thioalkyl is preferred.

The term “sulfonamide,” as used herein, means —S(O)₂ NR^{K}— or —NR^{K}S(O)—, wherein R^{K} may be hydrogen, alkyl, cycloalkyl, aryl, heteroaryl, heterocycle, alkenyl, or heteroalkyl.

The term

### [Chem. 30]

[symbol]

designates a single bond

### [Chem. 31]

(—)
or a double bond

### [Chem. 32]

(═).

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

The term “activator” as used herein refers to a molecular entity (e.g., but not limited to, a ligand and a disclosed compound) that enhances the activity of the target receptor protein.

The term “inhibitor” as used herein refers to a molecular entity (e.g., but not limited to, a disclosed compound) that decreases or disappears the activity of the target receptor protein.

The term “ligand” as used herein refers to a natural or synthetic molecular entity that is capable of associating or binding to a receptor to form a complex and mediate, prevent or modify a biological effect. Thus, the term “ligand” encompasses allosteric modulators, inhibitors, activators, agonists, antagonists, natural substrates and analogs of natural substrates.

The terms “natural ligand” and “endogenous ligand” as used herein are used interchangeably, and refer to a naturally occurring ligand, found in nature, which binds to a receptor.

The term “thallium flux assay” herein refers to a fluorescence-based assay used to monitor the activity of TREK channels. Thallium is a congener of potassium that readily fluxes through the pore of TREK channels. Thallium flux is measured using a commercially available, thallium-sensitive fluorescent dye called Thallos. The detail method is described below.

The term “patch clamp technique” herein refers to the “gold standard” technique for evaluating TREK channel pharmacology. The detail method is described below.

The term “MK-801-induced novel object recognition test” herein refers to the experiment to evaluate in vivo efficacy in the schizophrenic cognitive impairment animal model. The detail method is described below. MK-801 is also known as dizocilpine.

The term “TREK inhibitor” as used herein refers to any exogenously administered compound or agent that directly or indirectly inhibits the channel in an animal, in particular a mammal, for example a human.

The term “TREK activator” as used herein refers to any exogenously administered compound or agent that directly or indirectly activates the channel in an animal, in particular a mammal, for example a human.

The term “TREK modulator” as used herein refers to any exogenously administered compound or agent that directly or indirectly activates or inhibits the channel in an animal, in particular a mammal, for example a human. For clarity, the term “TREK modulator” as used herein refers to any TREK activator or TREK inhibitor.

The term “dysfunction” as used herein refers to any abnormal functions that induce activation or inhibition of the channel in an animal, in particular a mammal, for example a human.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Compounds

In one aspect, disclosed is a compound of formula (I):

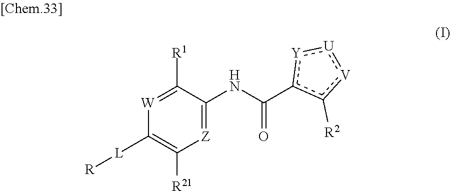

or a pharmaceutically acceptable salt thereof, wherein: all symbols are defined as below.

The compound of formula (I) is a modulator of TREK1, TREK2 or both TREK1/TREK2.

In some embodiments, the compound is a compound of formula (Ia):

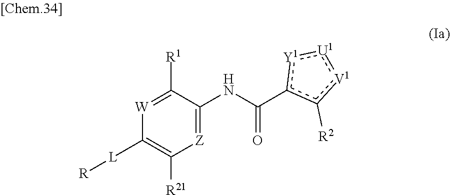

or a pharmaceutically acceptable salt thereof,
wherein all symbols are defined as below.

The compound of formula (Ia) is an inhibitor of TREK1, TREK2 or both TREK1/TREK2.

In some embodiments, the formula (Ia) is preferably the formula (Ia-1):

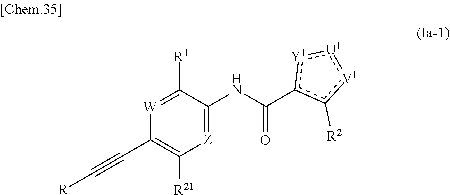

(wherein all symbols are as defined as below).

In some embodiments, the formula (Ia) is preferably the formula (Ia-1-4):

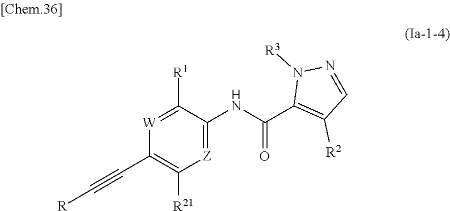

(wherein all symbols are as defined as below).

In some embodiments, the formula (Ia) is preferably the formula (Ia-1-5):

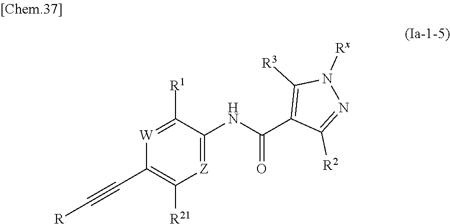

(wherein all symbols are as defined as below).

In some embodiments, the formula (Ia) is preferably the formula (Ia-2):

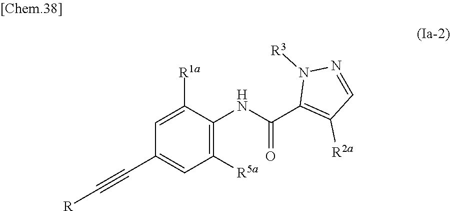

(wherein all symbols are as defined as below).

In some embodiments, the formula (Ia) is preferably the formula (Ia-3):

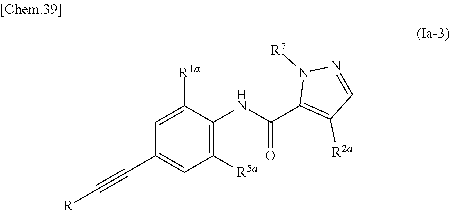

(wherein a symbols are as defined as below).

In some embodiments, the formula (Ia) is preferably the formula (Ia-4):

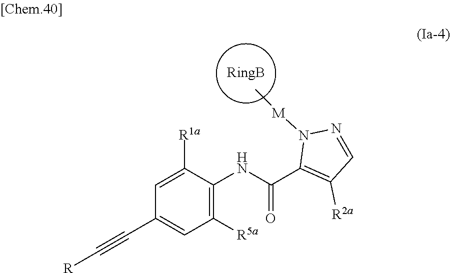

(wherein all symbols are as defined as below).

In some embodiments, the formula (Ia) is preferably the formula (Ia-5):

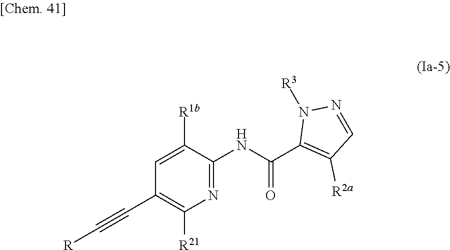

(wherein all symbols are as defined as below).

In some embodiments, the formula (Ia) is preferably the formula (Ia-6):

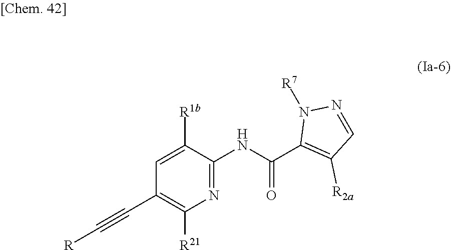

(wherein all symbols are as defined as below).

In some embodiments, the formula (Ia) is preferably the formula (Ia-7):

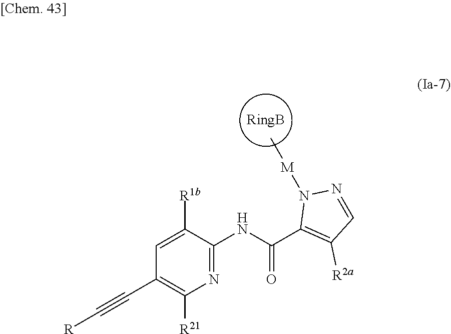

(wherein all symbols are as defined as below).

In some embodiments, the formula (Ta) is preferably the formula (Ia-1-1):

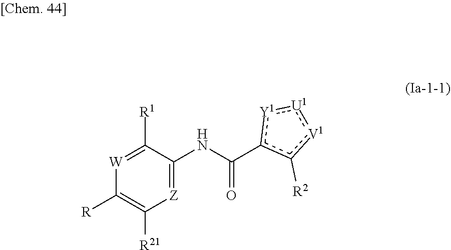

(wherein all symbols are as defined as below).

In some embodiments, the formula (Ia) is preferably the formula (Ia-1-1a):

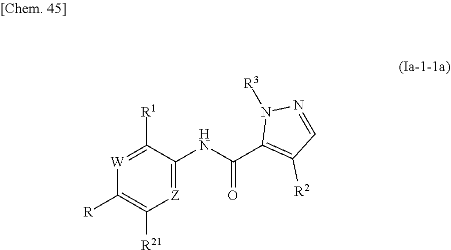

(wherein a symbols are as defined as below).

In some embodiments, the formula (Ia) is preferably the formula (Ia-1-2):

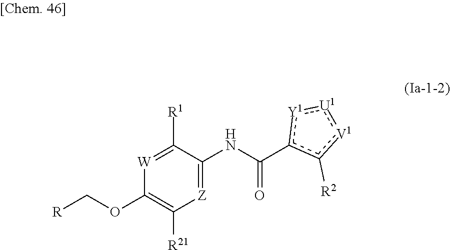

(wherein all symbols are as defined as below).

In some embodiments, the formula (Ia) is preferably the formula (Ia-1-2a):

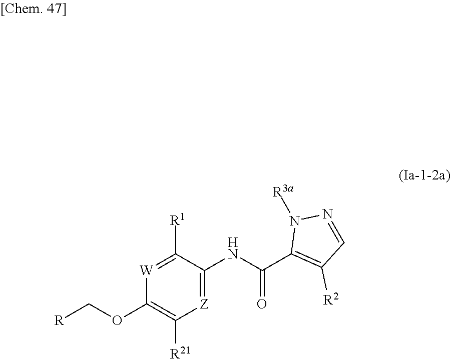

(wherein R^{3a} is selected form (1) R^{7a} or

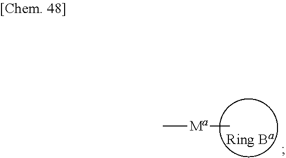

wherein R^{7a} is selected from (1) C1-C10-alkylene-cyano, (2) C1-C10-alkylene-NH—C(═O)—O—C1-C10-alkyl, (3) C1-10-alkylene-O—C1-C10-alkyl and (4) C1-C10-alkylene-(CR⁷¹R⁷²)—R⁷³ each of (1)-(4) is may be optionally substituted with 1 to 10 halogen,
M^{a} is selected from (1) bond or (2) C1-C10-alkylene which may be optionally substituted with 1 to 3 halogens;
Ring B^{a} is selected from (1) C3-C10-cycloalkyl which may be optionally substituted with 1 to 5 R⁸, (2) 3 to 15 membered heterocycle which may be optionally substituted with 1 to 5 R⁹, (3) 6 to 15 membered aryl which may be optionally substituted with 1 to 5 R¹⁰ and (4) 5 to 15 membered heteroaryl which may be optionally substituted with 1 to 5 R¹¹;
and the other symbols are as defined as below).

In some embodiments, the formula (Ia) is preferably the formula (Ia-1-3):

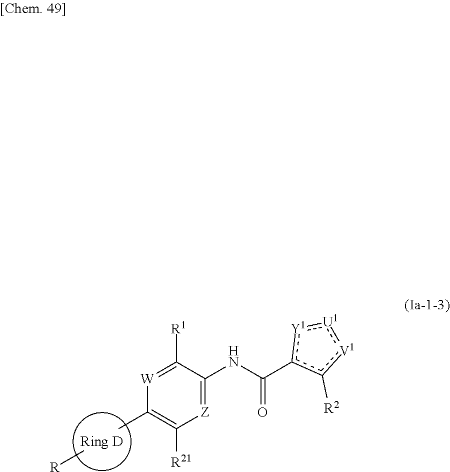

(wherein Ring D is selected from (1) 6 to 15 membered aryl and (2) 5 to 15 membered heteroaryl, and the other symbols are as defined as below).

In some embodiments, the formula (Ia) is preferably the formula (Ia-1-7a):

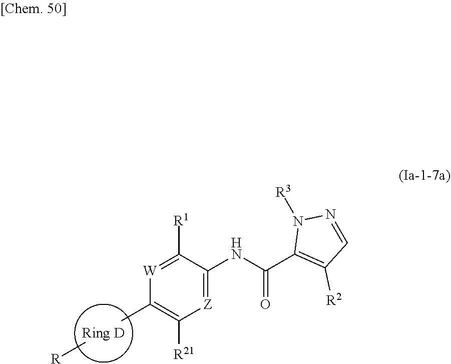

(wherein all symbols are as defined as below).

In some embodiments, the formula (Ia) is preferably the formula (Ia-1-3a):

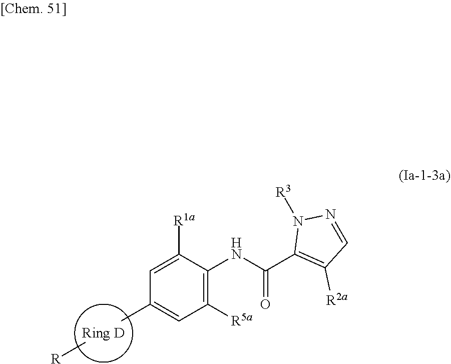

(wherein all symbols are as defined as below).

In some embodiments, the formula (Ia) is preferably the formula (Ia-1-3b):

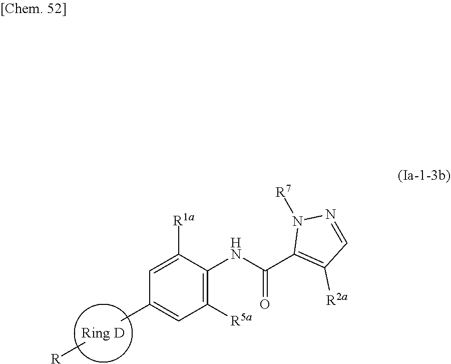

(wherein all symbols are as defined as below).

In some embodiments, the formula (Ia) is preferably the formula (Ia-1-3c):

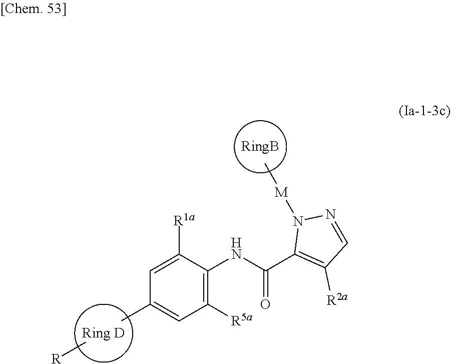

(wherein all symbols are as defined as below).

In some embodiments, the formula (Ia) is preferably the formula (Ia-1-3d):

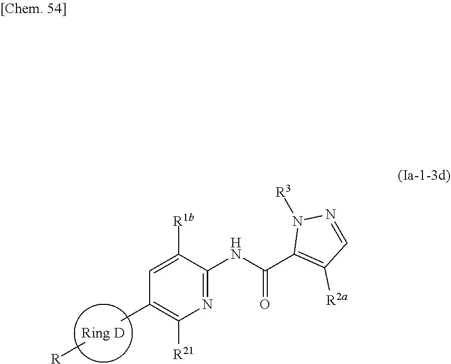

(wherein all symbols are as defined as below).

In some embodiments, the formula (Ia) is preferably the formula (Ia-1-3e):

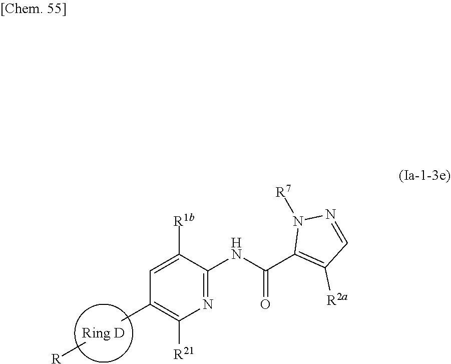

(wherein all symbols are as defined as below).

In some embodiments, the formula (Ia) is preferably the formula (Ia-1-3f):

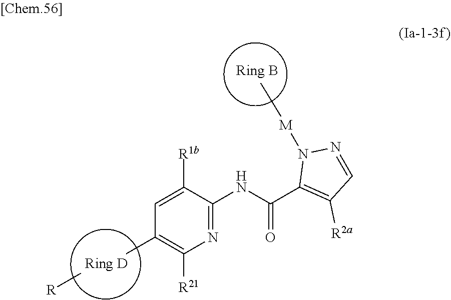

(wherein all symbols are as defined as below).

In some embodiments, the formula (Ia) is preferably the formula (Ia-1-6):

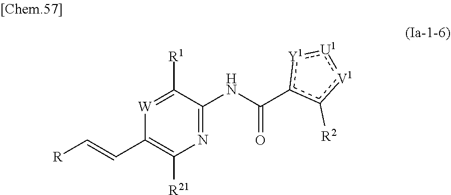

(wherein all symbols are as defined as below).

In some embodiments, the formula (Ia) is preferably the formula (1a-1-6a):

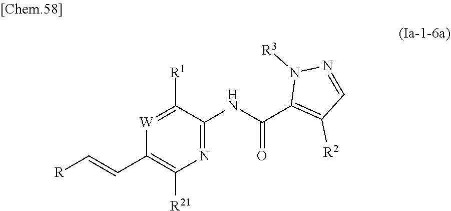

(wherein all symbols are as defined as below).

In some embodiments, the formula (I) is preferably the formula (Ia-1-4a), (Ia-1-7b) or (Ia-1-2b):

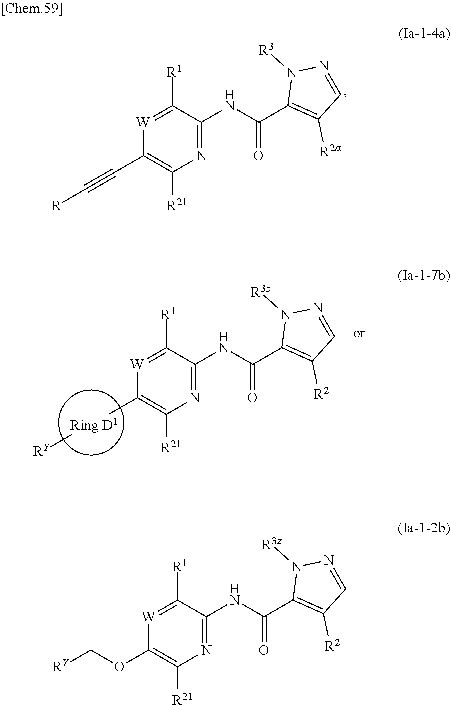

or a pharmaceutically acceptable salt thereof,
wherein Ring D¹ is selected from

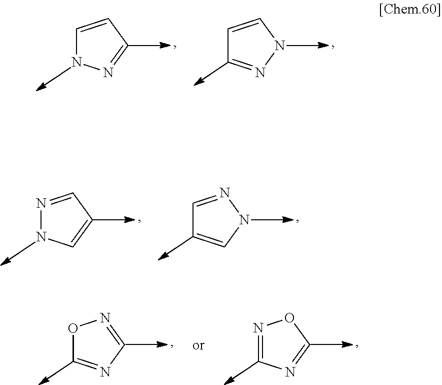 [SMILES: c1cn[nH]c1 ; c1cn[nH]c1 ; c1cn[nH]c1 ; c1cn[nH]c1 ; c1ncon1 ; c1ncon1]

wherein right arrow is connecting position with 6 membered ring and left arrow is connecting position with R^{Y} and the other symbols are defined as below.

In some embodiments, the formula (I) is preferably the formula (Ia-1-4b), (Ja-1-7b) or (Ia-1-2b):

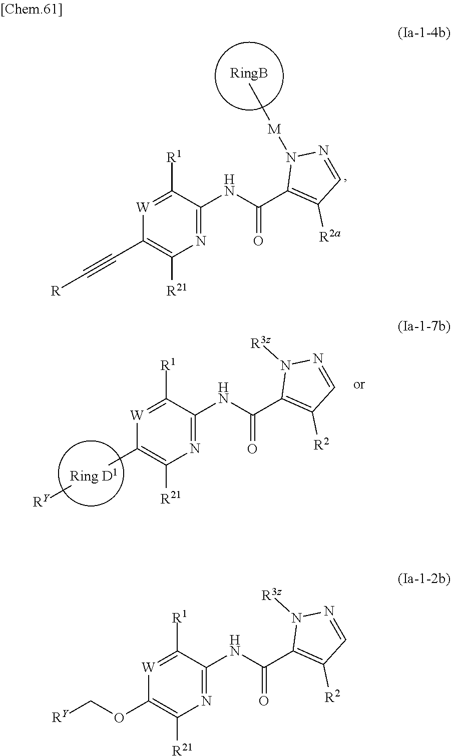

or a pharmaceutically acceptable salt thereof,
wherein Ring D¹ is selected from

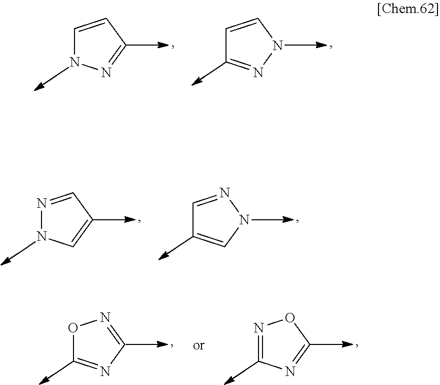 [SMILES: c1cn[nH]c1 ; c1cn[nH]c1 ; c1cn[nH]c1 ; c1cn[nH]c1 ; c1ncon1 ; c1ncon1]

wherein right arrow is connecting position with 6 membered ring and left arrow is connecting position with R^{Y} and the other symbols are defined as below.

In some embodiments, the compound of formula (I) is a compound of formula (Ib):

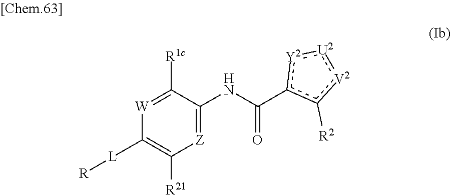

or a pharmaceutically acceptable salt thereof,
wherein all symbols are defined as below.

The compound of formula (Ib) is an activator of TREK1, TREK2 or both TREK1/TREK2.

In some embodiments, the formula (Ib) is preferably the formula (Ib-1):

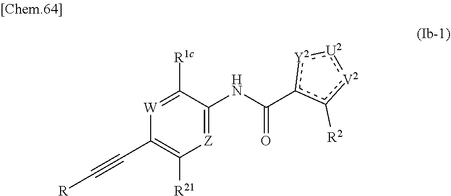

(wherein all symbols are as defined as below).

In some embodiments, the formula (Ib) is preferably the formula (Ib-2):

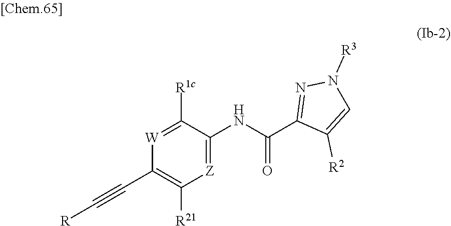

(wherein all symbols are as defined as below).

In some embodiments, the formula (Tb) is preferably the formula (Ib-2-1):

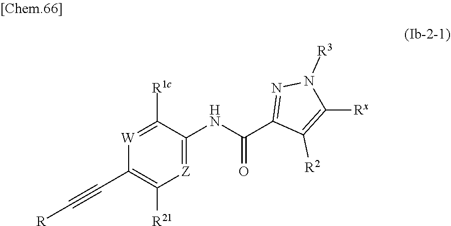

(wherein all symbols are as defined as below).

In some embodiments, the formula (Tb) is preferably the formula (Ib-3):

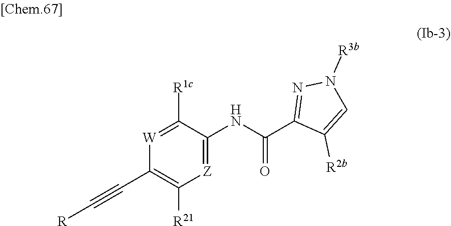

(wherein all symbols are as defined as below).

L is preferably bond, C2-C4-alkynylene (preferably, ethynylene), C2-C4-alkenylene(preferably, ethenylene), —(C1-C4-alkylene)-O— (preferably, —CH₂—O—), -(6 to 10 membered aryl)- or -(5 to 10 membered heteroaryl)-. More preferable L is C2-C4-alkynylene (preferably, ethynylene), —(C1-C4-alkylene)-O— (preferably, —CH₂—O—) or -(5 to 10 membered heteroaryl)-(preferably, 5 membered heteroaryl). Most preferable L is C2-C4-alkynylene (preferably, ethynylene). Most preferable L is also -(5 to 10 membered heteroaryl)-.

W is preferably CH, or CR⁴. More preferable W is CH.

Z is preferably CR⁸ or N. More preferable Z is N.

R⁴ is preferably cyano, halogen or C1-C4-alkyl which may be optionally substituted with 1 to 5 halogen. More preferable R⁴ is halogen or C1-C4-alkyl which may be optionally substituted with 1 to 5 halogen. Most preferable R⁴ is halogen or methyl.

R⁸ is preferably cyano, halogen or C1-C4-alkyl which may be optionally substituted with 1 to 5 halogen. More preferable R⁸ is halogen or C1-C4-alkyl which may be optionally substituted with 1 to 5 halogen. Most preferable R⁸ is halogen or methyl. Especially preferable R⁸ is methyl.

R^{b} is preferably hydrogen, C1-C4-alkyl or C1-C4-haloalkyl. More preferable R^{b} is hydrogen or C1-C4-alkyl.

R^{c} is preferably hydrogen, C1-C4-alkyl or C1-C4-haloalkyl.

R^{d} is preferably hydrogen, C1-C4-alkyl or C1-C4-haloalkyl.

R^{c} and R^{d} are preferably attached with the carbon atom together and formed a C3-C10-cycloalkyl. More preferable R^{c} and R^{d} are attached with the carbon atom together and formed a C3-C7-cycloalkyl.

n is preferably 1, 2, 3 or 4.

Q is preferably halogen, cyano, 6 to 10 membered aryl which may be may be optionally substituted with 1 to 5 substituents selected from (1) halogen, (2) C1-C4-alkyl and (3) C1-C4-haloalkyl, 5 to 10 membered heteroaryl which may be may be optionally substituted with 1 to 5 substituents selected from (1) halogen, (2) C1-C4-alkyl and (3) C1-C4-haloalkyl, C3-C8-cycloalkyl which may be may be optionally substituted with 1 to 5 substituents selected from (1) halogen, (2) C1-C4-alkyl and (3) C1-C4-haloalkyl, or 4 to 6 membered heterocycle which may be may be optionally substituted with 1 to 3 substituents selected from (1) halogen, (2) C1-C4-alkyl and (3) C1-C4-haloalkyl.

More preferable Q is halogen, cyano, C3-C8-cycloalkyl which may be may be optionally substituted with 1 to 5 substituents selected from (1) halogen, (2) C1-C4-alkyl and (3) C1-C4-haloalkyl, or 4 to 6 membered heterocycle which may be may be optionally substituted with 1 to 3 substituents selected from (1) halogen, (2) C1-C4-alkyl and (3) C1-C4-haloalkyl.

R²⁰¹ is preferably hydrogen, C1-C4-alkyl or C1-C4-haloalkyl.

R²⁰² is preferably hydrogen, C1-C4-alkyl or C1-C4-haloalkyl.

R²⁰³ is preferably hydrogen, C1-C4-alkyl or C1-C4-haloalkyl.

R²⁰⁴ is preferably hydrogen, C1-C4-alkyl or C1-C4-haloalkyl.

R²⁰¹ is preferably hydrogen, C1-C4-alkyl or C1-C4-haloalkyl.

R²⁰⁶ is preferably hydrogen, C1-C4-alkyl or C1-C4-haloalkyl.

R²⁰⁷ is preferably hydrogen, C1-C4-alkyl or C1-C4-haloalkyl.

R²⁰⁸ is preferably hydrogen, C1-C4-alkyl or C1-C4-haloalkyl.

R²⁰⁹ is preferably hydrogen, C1-C4-alkyl or C1-C4-haloalkyl.

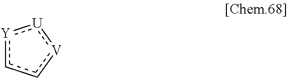

is preferably pyrrole substituted with R³ or pyrazole substituted with R³. More preferable

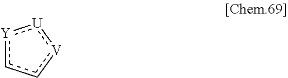

is pyrazole substituted with R³.

Y is preferably CH, CR³, N, NH or NR³. More preferable Y is CR³ or NR³. More preferable Y is also CH, N, or NH. Most preferable Y is NR³. Most preferable Y is also N or NH.

U is preferably CH, CR³, CR^{x}, N, NH, NR³ or NR^{x}. More preferable U is CH, CR^{x}, N, NH or NR^{x}. More preferable U is also CR³ or NR³. Most preferable U is NR³.

V is preferably CH, CR^{x}, N, NH or NR^{x}. More preferable V is CH, CR^{x}, N, NH or NR^{x}. Most preferable V is CH, N or NH.

R^{x} is preferably NH₂, halogen or methyl. More preferable R^{x} is NH₂ or methyl.

R is preferably 6 to 10 membered aryl which may be optionally substituted with 1 to 5 R⁶ or 5 to 10 membered heteroaryl which may be optionally substituted with 1 to 5 R⁶. More preferable R is 6 to 10 membered aryl which may be optionally substituted with 1 to 3 R⁶. More preferable R is also 5 membered heteroaryl which may be optionally substituted with 1 to 3 R⁶. Furthermore preferable R is thiophenyl or phenyl which may be optionally substituted with 1 to 3 R⁶. Most preferable R is phenyl which may be optionally substituted with 1 to 3 R⁶.

R⁶ is preferably halogen, cyano, C1-C4-alkyl which may be optionally substituted with 1 to 5 halogen or C1-C4-alkoxy which may be optionally substituted with 1 to 5 halogen. More preferable R⁶ is halogen, C1-C4-alkyl which may be optionally substituted with 1 to 5 halogen, C1-C4-alkoxy which may be optionally substituted with 1 to 5 halogen. Most preferable R⁶ is halogen or methyl which may be optionally substituted with 1 to 3 halogen.

R¹ is preferably C1-C4-alkyl, halogen, C1-C4-alkoxy, C1-C4-haloalkyl or C1-C4-haloalkoxy. More preferable R¹ is C1-C4-alkyl, C1-C4-haloalkyl or halogen. Further preferable R¹ is C1-C4-alkyl or halogen. Most preferable R¹ is methyl or halogen.

R²¹ is preferably hydrogen, C1-C4-alkyl, halogen, C1-C4-alkoxy, C1-C4-haloalkyl or C1-C4-haloalkoxy. More preferable R²¹ is hydrogen.

R² is preferably halogen, C1-C4-alkyl or C1-C4-haloalkyl. More preferable R² is C1-C4-alkyl or halogen. Most preferable R² is halogen.

R³ is preferably
—(C2-C6-alkylene)-cyano which may be optionally substituted with 1 to 10 halogen,
—(C1-C6-alkylene)-NH—C(═O)—O—(C1-C6-alkyl) which may be optionally substituted with 1 to 10 halogen,
—(C1-C6-alkylene)-O—(C1-C6-alkyl) which may be optionally substituted with 1 to 10 halogen,
C3-C8-cycloalkyl which may be optionally substituted with 1 to 5 R¹,
3 to 10 membered heterocycle which may be optionally substituted with 1 to 5 R⁹,
6 to 10 membered aryl which may be optionally substituted with 1 to 5 R¹⁰ or
5 to 10 membered heteroaryl which may be optionally substituted with 1 to 5 R¹¹,
—(C1-C4-alkylene)—(C3-C8-cycloalkyl which may be optionally substituted with 1 to 5 R⁸),
—(C1-C4-alkylene)-(3 to 10 membered heterocycle which may be optionally substituted with 1 to 5 R⁹),
—(C1-C4-alkylene)-(6 to 10 membered aryl which may be optionally substituted with 1 to 5 R¹⁰) or
—(C1-C4-alkylene)-(5 to 10 membered heteroaryl which may be optionally substituted with 1 to 5 R¹¹).

More preferable R³ is
—(C2-C4-alkylene)-cyano which may be optionally substituted with 1 to 5 halogen,
—(C3-C6-alkylene)-NH—C(═O)—O—(C3-C6-alkyl) which may be optionally substituted with 1 to 10 halogen,
—(C3-C6-alkylene)-O—(C3-C6-alkyl) which may be optionally substituted with 1 to 10 halogen,
C3-C8-cycloalkyl which may be optionally substituted with 1 to 5 R⁸,
3 to 10 membered heterocycle which may be optionally substituted with 1 to 5 R⁹,
—(C1-C4-alkylene)—(C3-C8-cycloalkyl which may be optionally substituted with 1 to 5 R⁸), or
—(C1-C4-alkylene)-(3 to 10 membered heterocycle which may be optionally substituted with 1 to 5 R⁹).

Furthermore preferable R³ is
—(C2-C4-alkylene)-cyano which may be optionally substituted with 1 to 5 halogen,
—(C3-C6-alkylene)-NH—C(═O)—O—(C3-C6-alkyl) which may be optionally substituted with 1 to 10 halogen,
—(C3-C6-alkylene)-O—(C3-C6-alkyl) which may be optionally substituted with 1 to 10 halogen,
C3-C8-cycloalkyl which may be optionally substituted with 1 to 5 R⁸,
3 to 10 membered heterocycle which may be optionally substituted with 1 to 5 R⁹, —(C1-C2-alkylene)—(C3-C8-cycloalkyl which may be optionally substituted with 1 to 5 R⁸), or
—(C1-C2-alkylene)-(3 to 10 membered heterocycle which may be optionally substituted with 1 to 5 R⁹).

Most preferable R³ is

—(C2-C4-alkylene)-cyano which may be optionally substituted with 1 to 5 halogen (preferably —(C2-C4-alkylene)-cyano),

—(C3-C6-alkylene)-NH—C(═O)—O—(C3-C6-alkyl) which may be optionally substituted with 1 to 10 halogen,

—(C3-C6-alkylene)-O—(C3-C6-alkyl) which may be optionally substituted with 1 to 10 halogen (preferably —(C3-C6-alkylene)-O—(C3-C6-alkyl)),

C3-C8-cycloalkyl which may be optionally substituted with 1 to 5 R¹, 4 to 7 membered heterocycle which may be optionally substituted with 1 to 5 R⁹,

—(C1-C2-alkylene)—(C3-C8-cycloalkyl which may be optionally substituted with 1 to 5 R⁸), or

—(C1-C2-alkylene)-(4 to 7 membered heterocycle which may be optionally substituted with 1 to 5 R⁹).

R⁷ is preferably
—(C2-C6-alkylene)-cyano which may be optionally substituted with 1 to 10 halogen,
—(C1-C6-alkylene)-NH—C(═O)—O—(C1-C6-alkyl) which may be optionally substituted with 1 to 10 halogen or
—(C1-C6-alkylene)-O—(C1-C6-alkyl) which may be optionally substituted with 1 to 10 halogen.

More preferable R⁷ is
—(C2-C4-alkylene)-cyano,
—(C3-C6-alkylene)-NH—C(═O)—O—(C3-C6-alkyl) which may be optionally substituted with 1 to 10 halogen or
—(C3-C6-alkylene)-O—(C3-C6-alkyl).

Most preferable R⁷ is —(C2-C4-alkylene)-cyano.
R⁷¹ is preferably hydrogen, C1-C4-alkyl or C1-C4-haloalkyl. More preferable R⁷¹ is C1-C4-alkyl.
R⁷² is preferably hydrogen, C1-C4-alkyl or C1-C4-haloalkyl. More preferable R⁷² is C1-C4-alkyl.
R⁷¹ and R⁷² are preferably attached with the carbon atom together and formed a C3-C7-cycloalkyl.
p is preferably 1, 2, 3 or 4. More preferable p is 1.
R⁷ is preferably
C3-C8-cycloalkyl which may be optionally substituted with 1-10 substituents selected from (1) halogen, (2) C1-C4-alkyl and (3) C1-C4-haloalkyl, or
4 to 6 membered heterocycle which may be optionally substituted with 1-5 substituents selected from (1) halogen, (2) C1-C4-alkyl and (3) C1-C4-haloalkyl.

R¹⁰¹ is preferably C1-C4-alkyl, or C1-C4-haloalkyl.

R¹⁰² is preferably hydrogen, C1-C4-alkyl, or C1-C4-haloalkyl.

R¹⁰³ is preferably hydrogen, C1-C4-alkyl, or C1-C4-haloalkyl.

R¹⁰⁴ is preferably hydrogen, C1-C4-alkyl, or C1-C4-haloalkyl.

R¹⁰⁵ is preferably hydrogen, C1-C4-alkyl, or C1-C4-haloalkyl.

R¹⁰⁶ is preferably hydrogen, C1-C4-alkyl, or C1-C4-haloalkyl.

R¹⁰⁷ is preferably hydrogen, C1-C4-alkyl, or C1-C4-haloalkyl.

R¹⁰⁸ is preferably hydrogen, C1-C4-alkyl, or C1-C4-haloalkyl.

R¹⁰⁹ is preferably hydrogen, C1-C4-alkyl, or C1-C4-haloalkyl.

M of

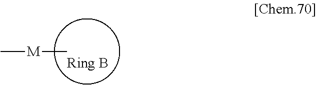

is preferably bond or C1-C4-alkylene which may be optionally substituted with 1 to 3 halogen.

More preferable M is bond or C1-C2-alkylene which may be optionally substituted with 1 to 3 halogen.

Ring B of

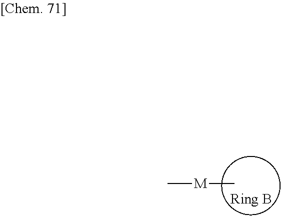

is preferably
C3-C8-cycloalkyl which may be optionally substituted with 1 to 5 R¹,
3 to 10 membered heterocycle which may be optionally substituted with 1 to 5 R⁹ or 5 to 10 membered heteroaryl which may be optionally substituted with 1 to 5 R¹¹.

More preferable Ring B is
C3-C8-cycloalkyl which may be optionally substituted with 1 to 5 R¹ or

3 to 10 membered heterocycle which may be optionally substituted with 1 to 5 R⁹. Furthermore preferable Ring B is 4 to 7 membered heterocycle which may be optionally substituted with 1 to 3 R⁹.

Furthermore preferable Ring B is also azetidine, pyrrolidine, piperidine, piperazine, azepane, 1, 4-diazepane, morpholine, tetrahydrofuran, tetrahydropyran, 1,4-dioxane, 1,4-diazepane, homomorpholine, 7-oxabicyclo[2.2.1]heptane or 3-azabicyclo[3.1.1]heptane which may be optionally substituted with 1 to 3 R⁹

Most preferable Ring B is azetidine, pyrrolidine, piperidine, piperazine, azepane, 1,4-diazepane, morpholine, tetrahydrofuran, tetrahydropyran or 1,4-dioxane which may be optionally substituted with 1 to 3 R⁹.

Especially most preferable Ring B is azetidine, pyrrolidine, piperidine which may be optionally substituted with 1 to 3 R⁹.
R⁸ of Ring B is preferably halogen,
C1-C4-alkyl which may be optionally substituted with 1 to 5 substituents selected from (1) halogen, (2) —OH and (3) C1-C4-alkoxy,
cyano,
—C(═O)—(C1-C4-alkyl) which may be optionally substituted with 1 to 5 substituents selected from (1) halogen, (2) —OH and (3) C1-C4-alkoxy,
—C(═O)—(C3-C7-cycloalkyl which may be optionally substituted with 1 to 5 substituents selected from (1) halogen, (2) —OH, (3) C1-C4-alkyl, (4) C1-C4-alkoxy and (5) C1-C4-haloalkyl),
—C(═O)—(3 to 10 membered heterocycle which may be optionally substituted with 1 to 5 substituents selected from (1) halogen, (2) —OH, (3) C1-C4-alkyl, (4) C1-C4-alkoxy and (5) C1-C4-haloalkyl),
—C(═O)—O—(C1-C4-alkyl) which may be optionally substituted with 1 to 5 substituents selected from (1) halogen, (2) —OH and (3) C1-C4-alkoxy,
—C(═O)—NH—(C3-C7-cycloalkyl which may be optionally substituted with 1 to 5 substituents selected from (1) halogen, (2) —OH, (3) C1-C4-alkyl, (4) C1-C4-alkoxy and (5) C1-C4-haloalkyl),
—NH—C(═O)—(C1-C4-alkyl) which may be optionally substituted with 1 to 5 substituents selected from (1) halogen, (2) —OH and (3) C1-C4-alkoxy,
—NH—C(═O)—(C3-C7-cycloalkyl which may be optionally substituted with 1 to 5 substituents selected from (1) halogen, (2) —OH, (3) C1-C4-alkyl, (4) C1-C4-alkoxy and (5) C1-C4-haloalkyl),
—NH—C(═O)—(3 to 10 membered heterocycle which may be optionally substituted with 1 to 5 substituents selected from (1) halogen, (2) —OH, (3) C1-C4-alkyl, (4) C1-C4-alkoxy and (5) C1-C4-haloalkyl),
—NH—C(═O)—O—(C1-C4-alkyl) which may be optionally substituted with 1 to 5 substituents selected from (1) halogen, (2) —OH and (3) C1-C4-alkoxy, or
—SO₂—(C1-C4-alkyl) which may be optionally substituted with 1 to 5 substituents selected from (1) halogen, (2) —OH and (3) C1-C4-alkoxy.

More preferable R¹ is
halogen,
C1-C4-alkyl which may be optionally substituted with 1 to 5 substituents selected from (1) halogen, (2) —OH and (3) C1-C4-alkoxy,
cyano,
—NH—C(═O)—(C1-C4-alkyl) which may be optionally substituted with 1 to 5 substituents selected from (1) halogen, (2) —OH and (3) C1-C4-alkoxy,
—NH—C(═O)—(C3-C7-cycloalkyl which may be optionally substituted with 1 to 5 substituents selected from (1) halogen, (2) —OH, (3) C1-C4-alkyl, (4) C1-C4-alkoxy and (5) C1-C4-haloalkyl),
—NH—C(═O)—(4 to 6 membered heterocycle which may be optionally substituted with 1 to 5 substituents selected from (1) halogen, (2) —OH, (3) C1-C4-alkyl, (4) C1-C4-alkoxy and (5) C1-C4-haloalkyl), or
—NH—C(═O)—O—(C1-C4-alkyl) which may be optionally substituted with 1 to 5 substituents selected from (1) halogen, (2) —OH and (3) C1-C4-alkoxy.

Most preferable R⁸ is
cyano,
—NH—C(═O)-(methyl which may be optionally substituted with 1 to 3 substituents selected from (1) halogen, (2) —OH and (3) C1-C4-alkoxy), or
—NH—C(═O)—O—(C1-C4-alkyl) which may be optionally substituted with 1 to 5 substituents selected from (1) halogen, (2) —OH and (3) C1-C4-alkoxy.

R⁹ of Ring B is preferably halogen,
C1-C4-alkyl which may be optionally substituted with 1 to 5 substituents selected from (1) halogen, (2) —OH and (3) C1-C4-alkoxy,
cyano,
—C(═O)—(C1-C4-alkyl) which may be optionally substituted with 1 to 5 substituents selected from (1) halogen, (2) —OH and (3) C1-C4-alkoxy,
—C(═O)—(C3-C7-cycloalkyl which may be optionally substituted with 1 to 5 substituents selected from (1) halogen, (2) —OH, (3) C1-C4-alkyl, (4) C1-C4-alkoxy and (5) C1-C4-haloalkyl),
—C(═O)—(3 to 10 membered heterocycle which may be optionally substituted with 1 to 5 substituents selected from (1) halogen, (2) —OH, (3) C1-C4-alkyl, (4) C1-C4-alkoxy and (5) C1-C4-haloalkyl),
—C(═O)—O—(C1-C4-alkyl) which may be optionally substituted with 1 to 5 substituents selected from (1) halogen, (2) —OH and (3) C1-C4-alkoxy,
—C(═O)—NH—(C1-C4-alkyl) which may be optionally substituted with 1 to 5 substituents selected from (1) halogen, (2) —OH and (3) C1-C4-alkoxy,
—C(═O)—NH—(C3-C7-cycloalkyl which may be optionally substituted with 1 to 5 substituents selected from (1) halogen, (2) —OH, (3) C1-C4-alkyl, (4) C1-C4-alkoxy and (5) C1-C4-haloalkyl),
—NH—C(═O)—(C1-C4-alkyl) which may be optionally substituted with 1 to 5 substituents selected from (1) halogen, (2) —OH and (3) C1-C4-alkoxy,
—NH—C(═O)—(C3-C7-cycloalkyl which may be optionally substituted with 1 to 5 substituents selected from (1) halogen, (2) —OH, (3) C1-C4-alkyl, (4) C1-C4-alkoxy and (5) C1-C4-haloalkyl),
—NH—C(═O)—(3 to 10 membered heterocycle which may be optionally substituted with 1 to 5 substituents selected from (1) halogen, (2) —OH, (3) C1-C4-alkyl, (4) C1-C4-alkoxy and (5) C1-C4-haloalkyl),
—NH—C(═O)—O—(C1-C4-alkyl) which may be optionally substituted with 1 to 5 substituents selected from (1) halogen, (2) —OH and (3) C1-C4-alkoxy, or
—SO—(C1-C4-alkyl) which may be optionally substituted with 1 to 5 substituents selected from (1) halogen, (2) —OH and (3) C1-C4-alkoxy.

More preferable R⁹ is
halogen,
C1-C4-alkyl which may be optionally substituted with 1 to 5 substituents selected from (1) halogen, (2) —OH and (3) C1-C4-alkoxy,
cyano,
—C(═O)—(C1-C4-alkyl) which may be optionally substituted with 1 to 5 substituents selected from (1) halogen, (2) —OH and (3) C1-C4-alkoxy,
—C(═O)—(C3-C7-cycloalkyl which may be optionally substituted with 1 to 5 substituents selected from (1) halogen, (2) —OH, (3) C1-C4-alkyl, (4) C1-C4-alkoxy and (5) C1-C4-haloalkyl),
—C(═O)—(4 to 6 membered heterocycle which may be optionally substituted with 1 to 5 substituents selected from (1) halogen, (2) —OH, (3) C1-C4-alkyl, (4) C1-C4-alkoxy and (5) C1-C4-haloalkyl),
—C(═O)—O—(C1-C4-alkyl) which may be optionally substituted with 1 to 5 substituents selected from (1) halogen, (2) —OH and (3) C1-C4-alkoxy,
—C(═O)—NH—(C1-C4-alkyl) which may be optionally substituted with 1 to 5 substituents selected from (1) halogen, (2) —OH and (3) C1-C4-alkoxy,
—NH—C(═O)—(C1-C4-alkyl) which may be optionally substituted with 1 to 5 substituents selected from (1) halogen, (2) —OH and (3) C1-C4-alkoxy,
—NH—C(═O)—(C3-C7-cycloalkyl which may be optionally substituted with 1 to 5 substituents selected from (1) halogen, (2) —OH, (3) C1-C4-alkyl, (4) C1-C4-alkoxy and (5) C1-C4-haloalkyl),
—NH—C(═O)—(4 to 6 membered heterocycle which may be optionally substituted with 1 to 5 substituents selected from (1) halogen, (2) —OH, (3) C1-C4-alkyl, (4) C1-C4-alkoxy and (5) C1-C4-haloalkyl),
—NH—C(═O)—O—(C1-C4-alkyl) which may be optionally substituted with 1 to 5 substituents selected from (1) halogen, (2) —OH and (3) C1-C4-alkoxy, or
—SO₂—(C1-C4-alkyl) which may be optionally substituted with 1 to 5 substituents selected from (1) halogen, (2) —OH and (3) C1-C4-alkoxy.

Most preferable R⁹ is
halogen,
methyl which may be optionally substituted with 1 to 3 substituents selected from (1) halogen, (2) —OH and (3) C1-C4-alkoxy,
cyano,
—C(═O)—(C1-C4-alkyl) which may be optionally substituted with 1 to 5 substituents selected from (1) halogen, (2) —OH and (3) C1-C4-alkoxy,
—C(═O)—(C3-C7-cycloalkyl which may be optionally substituted with 1 to 5 substituents selected from (1) halogen, (2) —OH, (3) C1-C4-alkyl, (4) C1-C4-alkoxy and (5) C1-C4-haloalkyl),
—C(═O)—(4 to 6 membered heterocycle which may be optionally substituted with 1 to 5 substituents selected from (1) halogen, (2) —OH, (3) C1-C4-alkyl, (4) C1-C4-alkoxy and (5) C1-C4-haloalkyl),
—C(═O)—O—(C1-C4-alkyl) which may be optionally substituted with 1 to 5 substituents selected from (1) halogen, (2) —OH and (3) C1-C4-alkoxy,
—NH—C(═O)—(C1-C4-alkyl) which may be optionally substituted with 1 to 5 substituents selected from (1) halogen, (2) —OH and (3) C1-C4-alkoxy,
—NH—C(═O)—(C3-C7-cycloalkyl which may be optionally substituted with 1 to 5 substituents selected from (1) halogen, (2) —OH, (3) C1-C4-alkyl, (4) C1-C4-alkoxy and (5) C1-C4-haloalkyl),
—NH—C(═O)—(4 to 6 membered heterocycle which may be optionally substituted with 1 to 5 substituents selected from (1) halogen, (2) —OH, (3) C1-C4-alkyl, (4) C1-C4-alkoxy and (5) C1-C4-haloalkyl), or
—NH—C(═O)—O—(C1-C4-alkyl) which may be optionally substituted with 1 to 5 substituents selected from (1) halogen, (2) —OH and (3) C1-C4-alkoxy.

R¹⁰ of Ring B is preferably
halogen,
C1-C4-alkyl which may be optionally substituted with 1 to 5 substituents selected from (1) halogen, (2) —OH and (3) C1-C4-alkoxy,
cyano,
—C(═O)—(C1-C4-alkyl) which may be optionally substituted with 1 to 5 substituents selected from (1) halogen, (2) —OH and (3) C1-C4-alkoxy,
—C(═O)—(C3-C7-cycloalkyl which may be optionally substituted with 1 to 5 substituents selected from (1) halogen, (2) —OH, (3) C1-C4-alkyl, (4) C1-C4-alkoxy and (5) C1-C4-haloalkyl),
—C(═O)—(3 to 10 membered heterocycle which may be optionally substituted with 1 to 5 substituents selected from (1) halogen, (2) —OH, (3) C1-C4-alkyl, (4) C1-C4-alkoxy and (5) C1-C4-haloalkyl),
—C(═O)—O—(C1-C4-alkyl) which may be optionally substituted with 1 to 5 substituents selected from (1) halogen, (2) —OH and (3) C1-C4-alkoxy,
—C(═O)—NH—(C1-C4-alkyl) which may be optionally substituted with 1 to 5 substituents selected from (1) halogen, (2) —OH and (3) C1-C4-alkoxy,
—C(═O)—NH—(C3-C7-cycloalkyl which may be optionally substituted with 1 to 5 substituents selected from (1) halogen, (2) —OH, (3) C1-C4-alkyl, (4) C1-C4-alkoxy and (5) C1-C4-haloalkyl),
—NH—C(═O)—(C1-C4-alkyl) which may be optionally substituted with 1 to 5 substituents selected from (1) halogen, (2) —OH and (3) C1-C4-alkoxy,
—NH—C(═O)—(C3-C7-cycloalkyl which may be optionally substituted with 1 to 5 substituents selected from (1) halogen, (2) —OH, (3) C1-C4-alkyl, (4) C1-C4-alkoxy and (5) C1-C4-haloalkyl),
—NH—C(═O)—(3 to 10 membered heterocycle which may be optionally substituted with 1 to 5 substituents selected from (1) halogen, (2) —OH, (3) C1-C4-alkyl, (4) C1-C4-alkoxy and (5) C1-C4-haloalkyl),
—NH—C(═O)—O—(C1-C4-alkyl) which may be optionally substituted with 1 to 5 substituents selected from (1) halogen, (2) —OH and (3) C1-C4-alkoxy, or
—SO₂—(C1-C4-alkyl) which may be optionally substituted with 1 to 5 substituents selected from (1) halogen, (2) —OH and (3) C1-C4-alkoxy.

More preferable R¹⁰ is
halogen,
C1-C4-alkyl which may be optionally substituted with 1 to 5 substituents selected from (1) halogen, (2) —OH and (3) C1-C4-alkoxy,
cyano,
—C(═O)—(C1-C4-alkyl) which may be optionally substituted with 1 to 5 substituents selected from (1) halogen, (2) —OH and (3) C1-C4-alkoxy, —C(═O)—(C3-C7-cycloalkyl which may be optionally substituted with 1 to 5 substituents selected from (1) halogen, (2) —OH, (3) C1-C4-alkyl, (4) C1-C4-alkoxy and (5) C1-C4-haloalkyl),
—C(═O)—(4 to 6 membered heterocycle which may be optionally substituted with 1 to 5 substituents selected from (1) halogen, (2) —OH, (3) C1-C4-alkyl, (4) C1-C4-alkoxy and (5) C1-C4-haloalkyl),
—C(═O)—O—(C1-C4-alkyl) which may be optionally substituted with 1 to 5 substituents selected from (1) halogen, (2) —OH and (3) C1-C4-alkoxy,
—C(═O)—NH—(C1-C4-alkyl) which may be optionally substituted with 1 to 5 substituents selected from (1) halogen, (2) —OH and (3) C1-C4-alkoxy,
—NH—C(═O)—(C1-C4-alkyl) which may be optionally substituted with 1 to 5 substituents selected from (1) halogen, (2) —OH and (3) C1-C4-alkoxy,
—NH—C(═O)—(C3-C7-cycloalkyl which may be optionally substituted with 1 to 5 substituents selected from (1) halogen, (2) —OH, (3) C1-C4-alkyl, (4) C1-C4-alkoxy and (5) C1-C4-haloalkyl),
—NH—C(═O)—(4 to 6 membered heterocycle which may be optionally substituted with 1 to 5 substituents selected from (1) halogen, (2) —OH, (3) C1-C4-alkyl, (4) C1-C4-alkoxy and (5) C1-C4-haloalkyl),
—NH—C(═O)—O—(C1-C4-alkyl) which may be optionally substituted with 1 to 5 substituents selected from (1) halogen, (2) —OH and (3) C1-C4-alkoxy, or
—SO₂—(C1-C4-alkyl) which may be optionally substituted with 1 to 5 substituents selected from (1) halogen, (2) —OH and (3) C1-C4-alkoxy.

Most preferable R¹⁰ is
halogen,
C1-C4-alkyl which may be optionally substituted with 1 to 5 substituents selected from (1) halogen, (2) —OH and (3) C1-C4-alkoxy, or
cyano.

R¹¹ of Ring B is preferably
halogen,
C1-C4-alkyl which may be optionally substituted with 1 to 5 substituents selected from (1) halogen, (2) —OH and (3) C1-C4-alkoxy,
cyano,
—C(═O)—(C1-C4-alkyl) which may be optionally substituted with 1 to 5 substituents selected from (1) halogen, (2) —OH and (3) C1-C4-alkoxy,
—C(═O)—(C3-C7-cycloalkyl which may be optionally substituted with 1 to 5 substituents selected from (1) halogen, (2) —OH, (3) C1-C4-alkyl, (4) C1-C4-alkoxy and (5) C1-C4-haloalkyl),
—C(═O)—(3 to 10 membered heterocycle which may be optionally substituted with 1 to 5 substituents selected from (1) halogen, (2) —OH, (3) C1-C4-alkyl, (4) C1-C4-alkoxy and (5) C1-C4-haloalkyl),
—C(═O)—O—(C1-C4-alkyl) which may be optionally substituted with 1 to 5 substituents selected from (1) halogen, (2) —OH and (3) C1-C4-alkoxy,
—C(═O)—NH—(C1-C4-alkyl) which may be optionally substituted with 1 to 5 substituents selected from (1) halogen, (2) —OH and (3) C1-C4-alkoxy,
—C(═O)—NH—(C3-C7-cycloalkyl which may be optionally substituted with 1 to 5 substituents selected from (1) halogen, (2) —OH, (3) C1-C4-alkyl, (4) C1-C4-alkoxy and (5) C1-C4-haloalkyl),
—NH—C(═O)—(C1-C4-alkyl) which may be optionally substituted with 1 to 5 substituents selected from (1) halogen, (2) —OH and (3) C1-C4-alkoxy,
—NH—C(═O)—(C3-C7-cycloalkyl which may be optionally substituted with 1 to 5 substituents selected from (1) halogen, (2) —OH, (3) C1-C4-alkyl, (4) C1-C4-alkoxy and (5) C1-C4-haloalkyl),
—NH—C(═O)—(3 to 10 membered heterocycle which may be optionally substituted with 1 to 5 substituents selected from (1) halogen, (2) —OH, (3) C1-C4-alkyl, (4) C1-C4-alkoxy and (5) C1-C4-haloalkyl),
—NH—C(═O)—O—(C1-C4-alkyl) which may be optionally substituted with 1 to 5 substituents selected from (1) halogen, (2) —OH and (3) C1-C4-alkoxy, or
—SO—(C1-C4-alkyl) which may be optionally substituted with 1 to 5 substituents selected from (1) halogen, (2) —OH and (3) C1-C4-alkoxy.

More preferable R¹¹ is
halogen,
C1-C4-alkyl which may be optionally substituted with 1 to 5 substituents selected from (1) halogen, (2) —OH and (3) C1-C4-alkoxy,
cyano,
—C(═O)—(C1-C4-alkyl) which may be optionally substituted with 1 to 5 substituents selected from (1) halogen, (2) —OH and (3) C1-C4-alkoxy,
—C(═O)—(C3-C7-cycloalkyl which may be optionally substituted with 1 to 5 substituents selected from (1) halogen, (2) —OH, (3) C1-C4-alkyl, (4) C1-C4-alkoxy and (5) C1-C4-haloalkyl),
—C(═O)—(4 to 6 membered heterocycle which may be optionally substituted with 1 to 5 substituents selected from (1) halogen, (2) —OH, (3) C1-C4-alkyl, (4) C1-C4-alkoxy and (5) C1-C4-haloalkyl),
—C(═O)—O—(C1-C4-alkyl) which may be optionally substituted with 1 to 5 substituents selected from (1) halogen, (2) —OH and (3) C1-C4-alkoxy,
—C(═O)—NH—(C1-C4-alkyl) which may be optionally substituted with 1 to 5 substituents selected from (1) halogen, (2) —OH and (3) C1-C4-alkoxy,
—NH—C(═O)—(C1-C4-alkyl) which may be optionally substituted with 1 to 5 substituents selected from (1) halogen, (2) —OH and (3) C1-C4-alkoxy,
—NH—C(═O)—(C3-C7-cycloalkyl which may be optionally substituted with 1 to 5 substituents selected from (1) halogen, (2) —OH, (3) C1-C4-alkyl, (4) C1-C4-alkoxy and (5) C1-C4-haloalkyl),
—NH—C(═O)—(4 to 6 membered heterocycle which may be optionally substituted with 1 to 5 substituents selected from (1) halogen, (2) —OH, (3) C1-C4-alkyl, (4) C1-C4-alkoxy and (5) C1-C4-haloalkyl),
—NH—C(═O)—O—(C1-C4-alkyl) which may be optionally substituted with 1 to 5 substituents selected from (1) halogen, (2) —OH and (3) C1-C4-alkoxy, or
—SO₂—(C1-C4-alkyl) which may be optionally substituted with 1 to 5 substituents selected from (1) halogen, (2) —OH and (3) C1-C4-alkoxy.

Most preferable R¹¹ is
halogen,
C1-C4-alkyl which may be optionally substituted with 1 to 5 substituents selected from (1) halogen, (2) —OH and (3) C1-C4-alkoxy, or
cyano.

R⁸¹ is preferably hydrogen.

R⁸² is preferably hydrogen.

R⁸³ is preferably hydrogen.

R⁸⁴ is preferably hydrogen.

R⁸⁵ is preferably hydrogen.

R⁸⁶ is preferably hydrogen.

R⁸⁷ is preferably hydrogen.

R⁸⁸ is preferably hydrogen.

R⁸⁹ is preferably hydrogen.

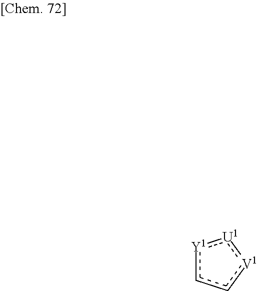

is preferably pyrrole substituted with R³ or pyrazole substituted with R³. More preferable

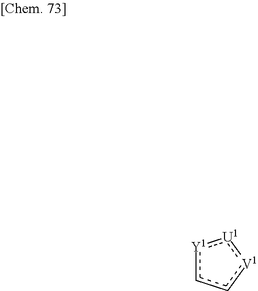

is pyrazole substituted with R³.

Y is preferably CR³ or NR³. More preferable Y¹ is NR³

U¹ is preferably CH, NR^{x} or N. More preferable U¹ is N.

V¹ is preferably CH or N. More preferable V¹ is CH.

R^{1a} is preferably halogen.

R^{1a} is also preferably C1-C4-alkyl. More preferable Ria is methyl.

R^{5a} is preferably hydrogen.

R^{5a} is also preferably halogen or C1-C4-alkyl. More preferable R^{5a} is halogen or methyl. Furthermore preferable R^{5d} is methyl.

R^{1b} is preferably halogen.

R^{1b} is also preferably C1-C4-alkyl. More preferable R^{1b} is methyl.

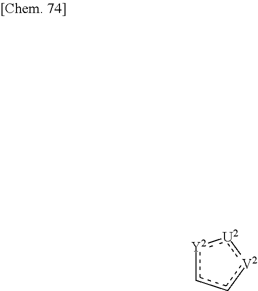

is preferably pyrazole substituted with R³.

Y² is preferably N.

U² is preferably NR³.

V² is preferably CH or CR^{x}. More preferable V² is CH.

R^{1c} is preferably C1-C4-alkyl or halogen. More preferable R^{1c} is methyl or halogen.

R^{3b} is preferably

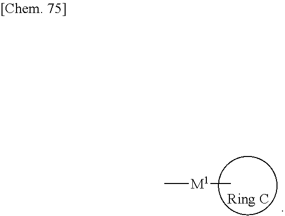

More preferable R^{3b} is —(C1-C10-alkylene)-(3 to 15 membered heterocycle which may be optionally substituted with 1 to 5 R¹⁴).

Furthermore preferable R^{3b} is —(C1-C4-alkylene)-(3 to 10 membered heterocycle which may be optionally substituted with 1 to 5 R¹⁴).

Most preferable R^{3b} is —(C1-C2-alkylene)-(3 to 10 membered heterocycle which may be optionally substituted with 1 to 5 R¹⁴)

M¹ of

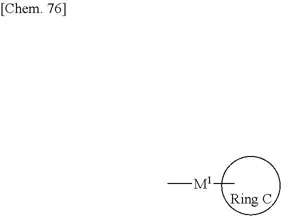

is preferably C1-C4-alkylene. More preferable M¹ is C1-C2-alkylene.

Ring C of

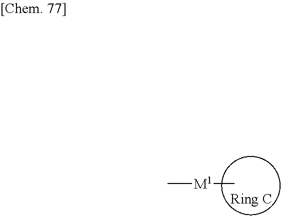

is preferably
C3-C7-cycloalkyl which may be optionally substituted with 1 to 5 R¹³, or 3 to 10 membered heterocycle which may be optionally substituted with 1 to 5 R¹⁴.

R¹³ is preferably C1-C4-alkyl, —C(═O)—(C1-C4-alkyl), —C(═O)—(C3-C7-cycloalkyl), —C(═O)—(3 to 10 membered heterocycle) or oxo.

R¹⁴ is preferably C1-C4-alkyl, —C(═O)—(C1-C4-alkyl), —C(═O)—(C3-C7-cycloalkyl), —C(═O)—(3 to 10 membered heterocycle) or oxo.

R¹⁵ is preferably C1-C4-alkyl, —C(═O)—(C1-C4-alkyl), —C(═O)—(C3-C7-cycloalkyl), —C(═O)—(3 to 10 membered heterocycle) or oxo.

R^{7a} of R^{3a} is preferably
—(C1-C6-alkylene)-cyano which may be optionally substituted with 1 to 5 halogen,
—(C1-C4-alkylene)-NH—C(═O)—O—(C1-C4-alkyl) which may be optionally substituted with 1 to 5 halogen, or
—(C1-C4-alkylene)-O—(C1-C4-alkyl) which may be optionally substituted with 1 to 5 halogen.

M^{a} of

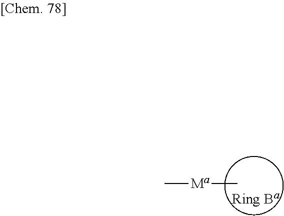

is preferably bond or —(C1-C6-alkylene)-. More preferable M^{a} is bond or —(C1-C4-alkylene)-.
Ring B^{a} of

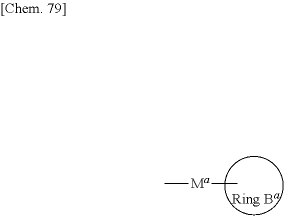

is preferably
C3-C10-cycloalkyl which may be optionally substituted with 1 to 5 R¹,
3 to 10 membered heterocycle which may be optionally substituted with 1 to 5 R⁹ or 5 to 10 membered heteroaryl which may be optionally substituted with 1 to 5 R¹¹.

More preferable Ring B^{a} is
C3-C10-cycloalkyl which may be optionally substituted with 1 to 5 R¹ or

3 to 10 membered heterocycle which may be optionally substituted with 1 to 5 R⁹.

Most preferable Ring B^{a} is 3 to 10 membered heterocycle which may be optionally substituted with 1 to 3 R⁹.

Ring D is preferably 5 to 10 membered heteroaryl. More preferable Ring D is pyrazolyl or oxadiazolyl is preferred. Most preferable Ring D is

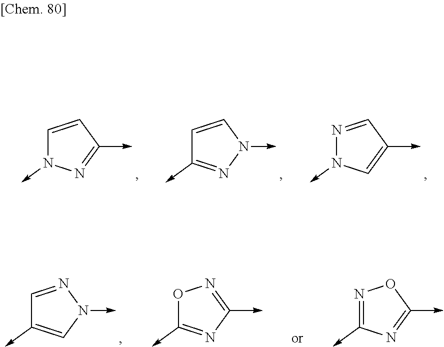

wherein right arrow is connecting position with 6 membered ring and left arrow is connecting position with R.

Ring D¹ is preferably pyrazolyl or oxadiazolyl. More preferable Ring D¹ is

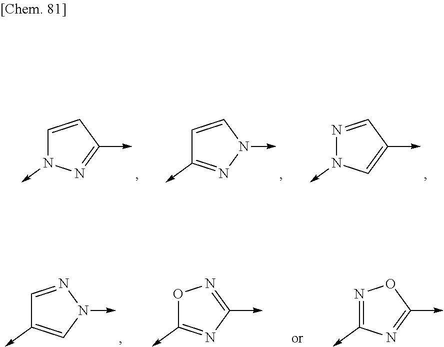

wherein right arrow is connecting position with 6 membered ring and left arrow is connecting position with R^{Y}. Most preferable 5 membered heteroaryl of Ring D¹ is

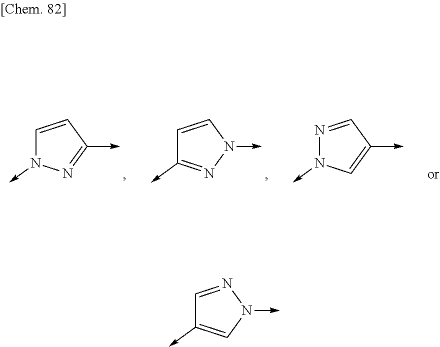

wherein right arrow is connecting position with 6 membered ring and left arrow is connecting position with R^{Y}.

In some embodiments, the formula (I) is also preferably the formula (Ia).

In some embodiments, the formula (I) is also preferably the formula (Ia-1).

In some embodiments, the formula (I) is also preferably the formula (Ia-1-1).

In some embodiments, the formula (I) is also preferably the formula (Ia-1-1a).

In some embodiments, the formula (I) is also preferably the formula (Ia-1-2).

In some embodiments, the formula (I) is also preferably the formula (Ia-1-2a).

In some embodiments, the formula (I) is also preferably the formula (Ia-1-2b).

In some embodiments, the formula (I) is also preferably the formula (Ia-1-3).

In some embodiments, the formula (I) is also preferably the formula (Ia-1-3a).

In some embodiments, the formula (I) is also preferably the formula (Ia-1-3b).

In some embodiments, the formula (I) is also preferably the formula (Ia-1-3c).

In some embodiments, the formula (I) is also preferably the formula (Ia-1-3d).

In some embodiments, the formula (I) is also preferably the formula (Ia-1-3e).

In some embodiments, the formula (I) is also preferably the formula (Ia-1-3f).

In some embodiments, the formula (I) is also preferably the formula (Ia-1-4).

In some embodiments, the formula (I) is also preferably the formula (Ia-1-4a).

In some embodiments, the formula (I) is also preferably the formula (Ia-1-4b).

In some embodiments, the formula (I) is also preferably the formula (Ia-1-5).

In some embodiments, the formula (I) is also preferably the formula (Ia-1-6).

In some embodiments, the formula (I) is also preferably the formula (Ia-1-6a).

In some embodiments, the formula (I) is also preferably the formula (Ia-1-7a).

In some embodiments, the formula (I) is also preferably the formula (Ia-1-7b).

In some embodiments, the formula (I) is also preferably the formula (Ia-2).

In some embodiments, the formula (I) is also preferably the formula (Ia-3).

In some embodiments, the formula (I) is also preferably the formula (Ia-4).

In some embodiments, the formula (I) is also preferably the formula (Ia-5).

In some embodiments, the formula (I) is also preferably the formula (Ia-6).

In some embodiments, the formula (I) is also preferably the formula (Ia-7).

In some embodiments, the formula (I) is also preferably the formula (Ib).

In some embodiments, the formula (I) is also preferably the formula (Ib-1).

In some embodiments, the formula (I) is also preferably the formula (Ib-2).

In some embodiments, the formula (I) is also preferably the formula (Ib-2-1).

In some embodiments, the formula (I) is also preferably the formula (Ib-3).

In some embodiments, the compound is preferably, but is not limited to:
(1) 1-((1,4-dioxan-2-yl)methyl)-4-chloro-N-(3-methyl-5-(phenylethynyl)pyridin-2-yl)-1H-pyrazole-5-carboxamide,
(2) 4-chloro-N-(3-methyl-5-(phenylethynyl)pyridin-2-yl)-1-((tetrahydro-2H-pyran-4-yl)methyl)-1H-pyrazole-5-carboxamide,
(3) (R)-4-chloro-N-(3-methyl-5-(phenylethynyl)pyridin-2-yl)-1-(tetrahydrofuran-3-yl)-1H-pyrazole-5-carboxamide
(4) (S)-1-((1,4-dioxan-2-yl)methyl)-4-chloro-N-(3-fluoro-5-(phenylethynyl)pyridin-2-yl)-1H-pyrazole-5-carboxamide,
(5) 1-(1-acetylpiperidin-4-yl)-4-chloro-N-(3-methyl-5-(phenylethynyl)pyridin-2-yl)-1H-pyrazole-5-carboxamide,
(6) 1-(1-acetylpiperidin-4-yl)-4-chloro-N-(3-fluoro-5-(phenylethynyl)pyridin-2-yl)-1H-pyrazole-5-carboxamide,
(7) 1-(1-acetylpiperidin-4-yl)-4-chloro-N-(3-fluoro-5-((4-fluorophenyl)ethynyl)pyridin-2-yl)-1H-pyrazole-5-carboxamide,
(8) 1-(1-acetylpiperidin-4-yl)-4-chloro-N-(5-((4-fluorophenyl)ethynyl)-3-methylpyridin-2-yl)-1H-pyrazole-5-carboxamide,
(9) 4-chloro-N-(3-methyl-5-(phenylethynyl)pyridin-2-yl)-1-(4,5,6,7-tetrahydropyrazolo[1,5-a]pyridin-5-yl)-1H-pyrazole-5-carboxamide,
(10) 1-((3R,4R)-1-acetyl-3-fluoropiperidin-4-yl)-4-chloro-N-(5-((4-fluorophenyl)ethynyl)-3-methylpyridin-2-yl)-1H-pyrazole-5-carboxamide,
(11) 1-(1-(1,4-dioxane-2-carbonyl)piperidin-4-yl)-4-chloro-N-(3-fluoro-5-(phenylethynyl)pyridin-2-yl)-1H-pyrazole-5-carboxamide,
(12) 1-(cis-4-acetamidocyclohexyl)-4-chloro-N-(3-methyl-5-(phenylethynyl)pyridin-2-yl)-1H-pyrazole-5-carboxamide,
(13) 1-(cis-4-acetamidocyclohexyl)-4-chloro-N-(3-fluoro-5-(phenylethynyl)pyridin-2-yl)-1H-pyrazole-5-carboxamide,
(14) 1-(cis-4-acetamidocyclohexyl)-4-chloro-N-(5-((4-fluorophenyl)ethynyl)-3-methylpyridin-2-yl)-1H-pyrazole-5-carboxamide,
(15) 1-((1-acetyl-4-fluoropiperidin-4-yl)methyl)-4-chloro-N-(3-methyl-5-(phenylethynyl)pyridin-2-yl)-1H-pyrazole-5-carboxamide,
(16) (S)-1-(1-(1-acetylpiperidin-4-yl)ethyl)-4-chloro-N-(3-methyl-5-(phenylethynyl)pyridin-2-yl)-1H-pyrazole-5-carboxamide,
(17) 1-((1-acetyl-3-fluoroazetidin-3-yl)methyl)-4-chloro-N-(3-fluoro-5-(phenylethynyl)pyridin-2-yl)-1H-pyrazole-5-carboxamide,
(18) 1-((1-acetyl-3-fluoroazetidin-3-yl)methyl)-4-chloro-N-(3-methyl-5-(phenylethynyl)pyridin-2-yl)-1H-pyrazole-5-carboxamide,
(19) 4-chloro-1-[(3S)-1-(cyclopropanecarbonyl)pyrrolidin-3-yl]-N-[3-methyl-5-(phenylethynyl)pyridin-2-yl]-1H-pyrazole-5-carboxamide,
(20) 4-chloro-1-[(3R)-1-(cyclopropanecarbonyl)pyrrolidin-3-yl]-N-[3-methyl-5-(phenylethynyl)pyridin-2-yl]-1H-pyrazole-5-carboxamide, or a pharmaceutically acceptable salt thereof.

In some embodiments, more preferable compound is
1-((1,4-dioxan-2-yl)methyl)-4-chloro-N-(3-methyl-5-(phenylethynyl)pyridin-2-yl)-1H-pyrazole-5-carboxamide,
or a pharmaceutically acceptable salt thereof.

In some embodiments, more preferable compound is
4-chloro-N-(3-methyl-5-(phenylethynyl)pyridin-2-yl)-1-((tetrahydro-2H-pyran-4-yl) methyl)-1H-pyrazole-5-carboxamide,
or a pharmaceutically acceptable salt thereof.

In some embodiments, more preferable compound is
(R)-4-chloro-N-(3-methyl-5-(phenylethynyl)pyridin-2-yl)-1-(tetrahydrofuran-3-yl)-1H-pyrazole-5-carboxamide,
or a pharmaceutically acceptable salt thereof.

In some embodiments, more preferable compound is
(S)-1-((1,4-dioxan-2-yl)methyl)-4-chloro-N-(3-fluoro-5-(phenylethynyl)pyridin-2-yl)-1H-pyrazole-5-carboxamide,
or a pharmaceutically acceptable salt thereof.

In some embodiments, more preferable compound is
1-(1-acetylpiperidin-4-yl)-4-chloro-N-(3-methyl-5-(phenylethynyl)pyridin-2-yl)-1H-pyrazole-5-carboxamide,
or a pharmaceutically acceptable salt thereof.

In some embodiments, more preferable compound is
1-(1-acetylpiperidin-4-yl)-4-chloro-N-(3-fluoro-5-(phenylethynyl)pyridin-2-yl)-1H-pyrazole-5-carboxamide,
or a pharmaceutically acceptable salt thereof.

In some embodiments, more preferable compound is
1-(1-acetylpiperidin-4-yl)-4-chloro-N-(3-fluoro-5-((4-fluorophenyl)ethynyl)pyridin-2-yl)-1H-pyrazole-5-carboxamide,
or a pharmaceutically acceptable salt thereof.

In some embodiments, more preferable compound is
1-(1-acetylpiperidin-4-yl)-4-chloro-N-(5-((4-fluorophenyl)ethynyl)-3-methylpyridin-2-yl)-1H-pyrazole-5-carboxamide,
or a pharmaceutically acceptable salt thereof.

In some embodiments, more preferable compound is
4-chloro-N-(3-methyl-5-(phenylethynyl)pyridin-2-yl)-1-(4,5,6,7-tetrahydropyrazolo[1,5-a]pyridin-5-yl)-1H-pyrazole-5-carboxamide,
or a pharmaceutically acceptable salt thereof.

In some embodiments, more preferable compound is
1-((3R,4R)-1-acetyl-3-fluoropiperidin-4-yl)-4-chloro-N-(5-((4-fluorophenyl)ethynyl)-3-methylpyridin-2-yl)-1H-pyrazole-5-carboxamide,
or a pharmaceutically acceptable salt thereof.

In some embodiments, more preferable compound is
1-(1-(1,4-dioxane-2-carbonyl)piperidin-4-yl)-4-chloro-N-(3-fluoro-5-(phenylethynyl) pyridin-2-yl)-1H-pyrazole-5-carboxamide,
or a pharmaceutically acceptable salt thereof.

In some embodiments, more preferable compound is
1-(cis-4-acetamidocyclohexyl)-4-chloro-N-(3-methyl-5-(phenylethynyl)pyridin-2-yl)-1H-pyrazole-5-carboxamide,
or a pharmaceutically acceptable salt thereof.

In some embodiments, more preferable compound is
1-(cis-4-acetamidocyclohexyl)-4-chloro-N-(3-fluoro-5-(phenylethynyl)pyridin-2-yl)-1H-pyrazole-5-carboxamide,
or a pharmaceutically acceptable salt thereof.

In some embodiments, more preferable compound is
1-(cis-4-acetamidocyclohexyl)-4-chloro-N-(5-((4-fluorophenyl)ethynyl)-3-methylpyridin-2-yl)-1H-pyrazole-5-carboxamide,
or a pharmaceutically acceptable salt thereof.

In some embodiments, more preferable compound is
1-((1-acetyl-4-fluoropiperidin-4-yl)methyl)-4-chloro-N-(3-methyl-5-(phenylethynyl) pyridin-2-yl)-1H-pyrazole-5-carboxamide,
or a pharmaceutically acceptable salt thereof.

In some embodiments, more preferable compound is
(S)-1-(1-(1-acetylpiperidin-4-yl)ethyl)-4-chloro-N-(3-methyl-5-(phenylethynyl)pyrid in-2-yl)-1H-pyrazole-5-carboxamide,
or a pharmaceutically acceptable salt thereof.

In some embodiments, more preferable compound is
1-((1-acetyl-3-fluoroazetidin-3-yl)methyl)-4-chloro-N-(3-fluoro-5-(phenylethynyl)pyridin-2-yl)-1H-pyrazole-5-carboxamide,
or a pharmaceutically acceptable salt thereof.

In some embodiments, more preferable compound is
1-((1-acetyl-3-fluoroazetidin-3-yl)methyl)-4-chloro-N-(3-methyl-5-(phenylethynyl)pyridin-2-yl)-1H-pyrazole-5-carboxamide,
or a pharmaceutically acceptable salt thereof.

In some embodiments, more preferable compound is
4-chloro-1-[(3S)-1-(cyclopropanecarbonyl)pyrrolidin-3-yl]-N-[3-methyl-5-(phenylethynyl)pyridin-2-yl]-1H-pyrazole-5-carboxamide,
or a pharmaceutically acceptable salt thereof.

In some embodiments, more preferable compound is
4-chloro-1-[(3R)-1-(cyclopropanecarbonyl)pyrrolidin-3-yl]-N-[3-methyl-5-(phenylethynyl)pyridin-2-yl]-1H-pyrazole-5-carboxamide,
or a pharmaceutically acceptable salt thereof.

The compound of the formula (I) is preferably such a compound that some or all of the above-mentioned preferred examples for R, L, W, Z, Y, U, V, R¹, R² and R²¹ are combined.

The compound of the formula (Ia) is preferably such a compound that some or all the above-mentioned preferred examples for R, L, W, Z, Y¹, U¹, V¹, R¹, R² and R²¹ are combined.

The compound of the formula (Ia-1) is preferably such a compound that some or all the above mentioned preferred examples for R, W, Z, Y¹, U¹, V¹, R¹, R² and R²¹ are combined.

The compound of the formula (Ia-1-1) is preferably such a compound that some or all of the above-mentioned preferred examples for R, W, Z, Y¹, U¹, V¹, R¹, R² and R²¹ are combined.

The compound of the formula (Ia-1-Ia) is preferably such a compound that some or all of the above-mentioned preferred examples for R, W, Z, R¹, R², R²¹ and R³ are combined.

The compound of the formula (Ia-1-2) is preferably such a compound that some or all of the above-mentioned preferred examples for R, W, Z, Y¹, U¹, V¹, R¹, R² and R²¹ are combined.

The compound of the formula (Ia-1-2a) is preferably such a compound that some or all of the above-mentioned preferred examples for R, W, Z, R¹, R², R²¹ and R^{3d} are combined.

The compound of the formula (Ia-1-2a) is preferably such a compound that some or all of the above-mentioned preferred examples for R^{Y}, W, Z, R¹, R², R²¹ and R^{3z} are combined.

The compound of the formula (Ia-1-3) is preferably such a compound that some or all of the above-mentioned preferred examples for R, Ring D, W, Z, Y¹, U¹, V¹, R¹, R² and R²¹ are combined.

The compound of the formula (Ia-1-3a) is preferably such a compound that some or all of the above-mentioned preferred examples for R, Ring D, Ria, R^{2a}, R³ and R^{5a} are combined.

The compound of the formula (Ia-1-3b) is preferably such a compound that some or all of the above-mentioned preferred examples for R, Ring D, Ria, R^{2a}, R^{5a} and R⁷ are combined.

The compound of the formula (Ia-1-3c) is preferably such a compound that some or all of the above-mentioned preferred examples for R, Ring B, Ring D, M, Ria, R^{2a} and R^{5a} are combined.

The compound of the formula (Ia-1-3d) is preferably such a compound that some or all of the above-mentioned preferred examples for R, Ring D, R^{1b}, R^{2a}, R²¹ and R³ are combined.

The compound of the formula (Ia-1-3e) is preferably such a compound that some or all of the above-mentioned preferred examples for R, Ring D, R^{1b}, R^{2a}, R²¹ and R⁷ are combined.

The compound of the formula (Ia-1-3f) is preferably such a compound that some or all of the above-mentioned preferred examples for R, Ring D, M, R^{1b}, R^{2a} and R²¹ are combined.

The compound of the formula (Ia-1-4) is preferably such a compound that some or all of the above-mentioned preferred examples for R, W, Z, R¹, R², R²¹ and R³ are combined.

The compound of the formula (Ia-1-5) is preferably such a compound that some or all of the above-mentioned preferred examples for R, W, Z, R¹, R², R²¹, R³ and R^{x} are combined.

The compound of the formula (Ia-1-6) is preferably such a compound that some or all of the above-mentioned preferred examples for R, W, Z, Y¹, U¹, V¹, R¹, R², R²¹, R³ and R^{x} are combined.

The compound of the formula (Ia-1-6a) is preferably such a compound that some or all of the above-mentioned preferred examples for R, W, Z, R¹, R^{2a}, R²¹, R³ and R^{x} are combined.

The compound of the formula (Ia-2) is preferably such a compound that some or all of the above-mentioned preferred examples for R, R^{1a}, R^{2a}, R³ and R^{5a} are combined.

The compound of the formula (Ia-3) is preferably such a compound that some or all of the above-mentioned preferred examples for R, R^{1a}, R^{2a}, R³, R^{5a} and R⁷ are combined.

The compound of the formula (Ia-4) is preferably such a compound that some or all of the above-mentioned preferred examples for R, Ring B, M, R^{1a}, R^{2a} and R^{5a} are combined.

The compound of the formula (Ia-5) is preferably such a compound that some or all of the above-mentioned preferred examples for R, R^{1b}, R^{2a} R²¹ and R³ are combined.

The compound of the formula (Ia-6) is preferably such a compound that some or all of the above-mentioned preferred examples for R, R^{1b}, R^{2a} R²¹ and R⁷ are combined.

The compound of the formula (Ia-7) is preferably such a compound that some or all of the above-mentioned preferred examples for R, Ring B, M, R^{1b}, R^{2a} and R²¹ are combined.

The compound of the formula (Ia-1-7a) is preferably such a compound that some or all of the above-mentioned preferred examples for R, Ring D, W, Z, R¹, R² and R²¹ are combined.

The compound of the formula (Ia-1-7b) is preferably such a compound that some or all of the above-mentioned preferred examples for R^{Y}, Ring D¹, W, Z, R¹, R², R²¹ and R^{3z} are combined.

The compound of the formula (Ib) is preferably such a compound that some or all of the above-mentioned preferred examples for R, L, W, Z, Y², U², V², R^{1c}, R², and R²¹ are combined.

The compound of the formula (Ib-1) is preferably such a compound that some or all of the above-mentioned preferred examples for R, W, Z, Y², U², V², R^{1c}, R², and R²¹ are combined.

The compound of the formula (Ib-2) is preferably such a compound that some or all of the above-mentioned preferred examples for R, W, Z, R^{1c}, R², R²¹ and R³ are combined.

The compound of the formula (Ib-2-1) is preferably such a compound that some or all of the above-mentioned preferred examples for R, W, Z, R^{1c}, R², R²¹, R³ and R^{x} are combined.

The compound of the formula (Ib-3) is preferably such a compound that some or all of the above-mentioned preferred examples for R, W, Z, R^{1c}, R^{2b}, R²¹ and R^{3b} are combined.

As inhibitor of TREK, the formula (I) is preferably the formula (Ia).

As activator of TREK, the formula (I) is preferably the formula (Ib).

As inhibitor of TREK, a disorder associated with TREK1, TREK2 or dual TREK1/TREK2 dysfunction in which inhibitors of TREK1, TREK2 or both TREK1 and TREK2 would offer therapeutic benefit is preferably neurological and/or psychiatric disorder.

More preferable disorder associated with TREK1, TREK2 or dual TREK1/TREK2 dysfunction in which inhibitors of TREK1, TREK2 or both TREK1 and TREK2 would offer therapeutic benefit is depression, schizophrenia, anxiety disorders, bipolar disorder, Alzheimer's disease, Parkinson's disease, Huntington's disease, Amyotrophic lateral sclerosis, 22q11.2 deletion syndrome, neuropathic pain or cerebral infarction.

Furthermore preferable disorder associated with TREK1, TREK2 or dual TREK1/TREK2 dysfunction in which inhibitors of TREK1, TREK2 or both TREK1 and TREK2 would offer therapeutic benefit is depression, schizophrenia, anxiety disorders, bipolar disorder. Most preferable disorder associated with TREK1, TREK2 or dual TREK1/TREK2 dysfunction in which inhibitors of TREK1, TREK2 or both TREK1 and TREK2 would offer therapeutic benefit is depression or schizophrenia.

As activator of TREK, a disorder associated with TREK1, TREK2 or dual TREK1/TREK2 dysfunction in which activators of TREK1, TREK2 or both TREK1 and TREK2 would offer therapeutic benefit is preferably pain, migraine, nasal inflammation, atrial fibrillation, acute respiratory distress syndrome, acute lung injury, overactive bladder, cerebral ischemia, epilepsy, amyotrophic lateral sclerosis, 22q11.2 deletion syndrome, neuronal degenerative diseases (e.g. Alzheimer's disease), sepsis, pancreatic cancer, Cushing's syndrome, autosomal dominant polycystic kidney disease, bone fracture, osteoporosis, temporal lobe epilepsy, schizophrenia, colitis, or addiction.

Compound names are assigned by using the Struct=Name naming algorithm as part of CHEMDRAW® ULTRA of PerkinElmer or ACD/Name® of Advanced Chemistry Development. These programs generally denominate a compound according to the IUPAC nomenclature. A compound names used in the present specification also are named according to the IUPAC nomenclature.

In the present invention, unless otherwise specified, the symbol:

### [Chem. 83]

[symbol]

represents that a substituent binds to the back side on the paper surface (in other words, α-configuration),
the symbol:

### [Chem. 84]

[symbol]

represents that a substituent binds to the front side on the paper surface (in other words, β-configuration),
the symbol:

### [Chem. 85]

[symbol]

represents α-configuration or β-configuration,
the symbol:

### [Chem. 86]

[symbol]

represents a mixture of α-configuration and β-configuration as long as the symbol is substituted at an asymmetric carbon atom.

The compound may exist as a stereoisomer wherein asymmetric or chiral centers are present. The stereoisomer is “R” or “S” depending on the configuration of substituents around the chiral carbon atom. The terms “R” and “S” used herein are configurations as defined in IUPAC 1974 Recommendations for Section E, Fundamental Stereochemistry, in Pure Appl. Chem., 1976, 45: 13-30. The disclosure contemplates various stereoisomers and mixtures thereof and these are specifically included within the scope of this invention. Stereoisomers include enantiomers and diastereomers, and mixtures of enantiomers or diastereomers. Individual stereoisomers of the compounds may be prepared synthetically from commercially available starting materials, which contain asymmetric or chiral centers or by preparation of racemic mixtures followed by methods of resolution well-known to those of ordinary skill in the art. These methods of resolution are exemplified by (1) attachment of a mixture of enantiomers to a chiral auxiliary, separation of the resulting mixture of diastereomers by recrystallization or chromatography and optional liberation of the optically pure product from the auxiliary as described in Furniss, Hannaford, Smith, and Tatchell, “Vogel's Textbook of Practical Organic Chemistry,” 5th edition (1989), Longman Scientific & Technical, Essex CM20 2JE, England, or (2) direct separation of the mixture of optical enantiomers on chiral chromatographic columns, or (3) fractional recrystallization methods.

It should be understood that the compound may possess tautomeric forms, as well as geometric isomers, and that these also constitute embodiments of the disclosure.

The present disclosure also includes an isotopically-labeled compound, which is identical to those recited in formula (I), but for the fact that one or more atoms are replaced by an atom having an atomic mass or mass number different from the atomic mass or mass number usually found in nature. Examples of isotopes suitable for inclusion in the compounds of the invention are hydrogen, carbon, nitrogen, oxygen, phosphorus, sulfur, fluorine, and chlorine, such as, but not limited to ²H, ³H, ¹³C, ¹⁴C, ¹⁵N, ¹⁸O, ¹⁷, ³¹p, ³²p, 35S, IF, and ³⁶Cl, respectively. Substitution with heavier isotopes can afford certain therapeutic advantages resulting from greater metabolic stability, for example increased in vivo half-life or reduced dosage requirements and, hence, may be preferred in some circumstances. The compound may incorporate positron-emitting isotopes for medical imaging and positron-emitting tomography (PET) studies for determining the distribution of receptors. Suitable positron-emitting isotopes that can be incorporated in compounds of formula (I) are ¹¹C, ¹³N, ¹⁵O, and ¹⁸F. Isotopically-labeled compounds of formula (I) can generally be prepared by conventional techniques known to those skilled in the art or by processes analogous to those described in the accompanying Examples using appropriate isotopically-labeled reagent in place of non-isotopically-labeled reagent.

Pharmaceutically Acceptable Salts

The disclosed compounds may exist as pharmaceutically acceptable salts. The term “pharmaceutically acceptable salt” refers to salts or zwitterions of the compounds which are water or oil-soluble or dispersible, suitable for treatment of disorders without undue toxicity, irritation, and allergic response, commensurate with a reasonable benefit/risk ratio and effective for their intended use. The salts may be prepared during the final isolation and purification of the compounds or separately by reacting an amino group of the compounds with a suitable acid. For example, a compound may be dissolved in a suitable solvent, such as but not limited to methanol and water and treated with at least one equivalent of an acid, like hydrochloric acid. The resulting salt may precipitate out and be isolated by filtration and dried under reduced pressure. Alternatively, the solvent and excess acid may be removed under reduced pressure to provide a salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetate, trifluoroacetate, glutamate, para-toluenesulfonate, undecanoate, hydrochloric, hydrobromic, sulfuric, phosphoric and the like. The amino groups of the compounds may also be quaternized with alkyl chlorides, bromides and iodides such as methyl, ethyl, propyl, isopropyl, butyl, lauryl, myristyl, stearyl and the like.

Basic addition salts may be prepared during the final isolation and purification of the disclosed compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts can be prepared, such as those derived from methylamine, dimethylamine, trimethylamine, triethylamine, diethylamine, ethylamine, tributylamine, pyridine, N,N-dimethylaniline, N-methylpiperidine, Nmethylmorpholine, dicyclohexylamine, procaine, dibenzylamine, N,N-dibenzylphenethylamine, 1-ephenamine and N,N′-dibenzylethylenediamine, ethylenediamine, ethanolamine, diethanolamine, piperidine, piperazine, and the like.

In the present invention, all the mentions of the compound of the present invention include a compound represented by formula (I), or a salt, a solvate, or a cocrystal thereof.

The compound represented by formula (I) and a salt thereof may be present in a not-solvation form, or in a solvation form with pharmaceutically acceptable solvent such as water or ethanol. Preferable solvates include hydrate. The compound represented by formula (I) and a salt thereof can be converted into a solvate by a well-known method.

The compound represented by formula (I) can form a cocrystal with an appropriate cocrystal former. As the cocrystal, pharmaceutically acceptable cocrystal that is formed with a pharmaceutically acceptable cocrystal former is preferable. The cocrystal is typically defined as a crystal that is formed of two or more different molecules by intermolecular interaction that is different from ionic bond. Furthermore, the cocrystal may be a composite of a neutral molecule and a salt. The cocrystal can be prepared by recrystallization from a solvent by a well-known method, for example, melting crystallization, or physically pulverizing the components together. Appropriate cocrystal formers include ones described in WO2006/007448.

The compound represented by the formula (I) can be administered as a prodrug. The prodrug of the compound represented by the formula (I) refers to a compound which is converted in vivo to the compound represented by the formula (I) by the reaction with enzymes, gastric acid and the like. Examples of the prodrug of the compound represented by the formula (I) include, when the compound represented by the formula (I) has an amino group, compounds in which the amino group is acylated, alkylated or phosphorylated (e.g. compounds represented by the formula (I) in which the amino group thereof is converted to eicosanoyl, aranyl, pentylaminocarbonyl, (5-methyl-2-oxo-1,3-dioxolen-4-yl)methoxycarbonyl, tetrahydrofuranyl, pyrrolidylmethyl, pivaloyloxymethyl, acetoxymethyl, tert-butyl or the like); when the compound represented by the formula (I) has a hydroxy group, compounds in which the hydroxy group is acylated, alkylated, phosphorylated or converted to borate (e.g. compounds represented by the formula (I) in which the hydroxy group thereof is converted to acetyl, palmitoyl, propanoyl, pivaloyl, succinyl, fumaryl, alanyl, dimethylaminomethylcarbonyl or the like); when the compound represented by the formula (I) has a carboxy group, compounds in which the carboxy group is esterified or amidated (e.g. compounds represented by the formula (I) in which the carboxy group thereof is converted to methyl ester, ethyl ester, isopropyl ester, phenyl ester, carboxymethyl ester, dimethylaminomethyl ester, pivaloyloxymethyl ester, phthalidyl ester, 1-{(ethoxycarbonyl)oxy}ethyl ester, (5-methyl-2-oxo-1,3-dioxolen-4-yl)methyl ester, 1-{[(cyclohexyloxy)carbonyl]oxy}ethyl ester, methylamide or the like) and the like. The prodrug of the compound represented by the formula (I) may be the one which is converted to the compound represented by the formula (I) under the physiological condition such as those disclosed in “Iyakuhin no Kaihatsu”, vol. 7 “Bunshi Sekkei”, p. 163-198, 1990, Hirokawa Shoten Co.

General Synthesis

Compounds of formula (I) may be prepared by synthetic processes or by metabolic processes. Preparation of the compounds by metabolic processes includes those occurring in the human or animal body (in vivo) or processes occurring in vitro.

Abbreviations which have been used in the descriptions of the Schemes that follow are: AcOH is acetic acid; DCE is 1,2-Dichloroethane; DIEA or DIPEA is N,N-diisopropylethylamine; DMF is N,N-dimethylformamide; IPA is isopropyl alcohol; HATU for 1-[bis(dimethylamino)methylene]-1H-1,2,3-triazolo[4,5-b]pyridinium 3-oxide hexafluorophosphate; MW is microwave (referring to a microwave reactor); and PyClU is 1-(chloro-1-pyrrolidinylmethylene)pyrrolidinium hexafluorophosphate.

Compounds of formula (I), specifically ethynylene-pyrazole compounds of A6, A8, A11, A12, A15 and A17, can be synthesized as shown in Scheme I to X.

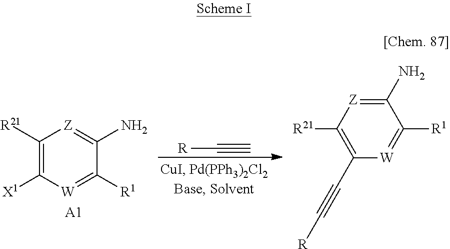

wherein X¹ is selected from (1) halogen, (2) tolsylate, (3) mesylate and (4)triflate and the other symbols are defined as below.

As shown in Scheme I, compounds A1 may be coupled with a variety of alkynes under Sonogashira coupling conditions, generally known in the art, to provide intermediates A2. For example the reaction may be conducted with a palladium catalyst such as Pd(PPh₃)₂Cl₂ in the presence of a base (e.g, DIEA) and a copper source (e.g., Copper (I) Iodide) in a solvent such as dimethylformamide with heating up to around 60° C.

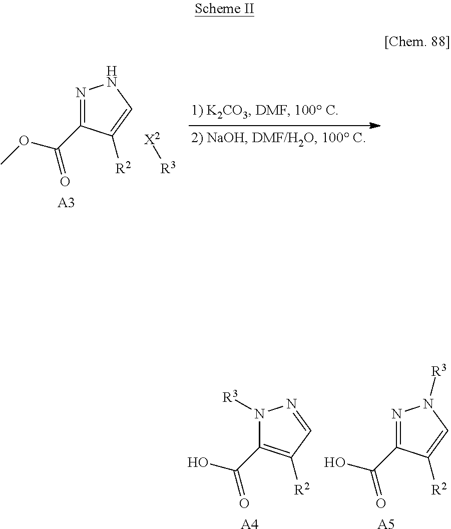

wherein X² is selected from (1) halogen, (2) tosylate, (3) mesylate and (4)triflate and the other symbols are defined as below.

Scheme II illustrates a general route to the intermediates A4 and regioisomer A5. Intermediate A3 may be alkylated with appropriate coupling partner (e.g. R³—X², where X² is halogen, tosylate, mesylate or triflate), a base (e.g. K₂CO₃) in solvent (e.g. DMF) under heat (e.g. around 100° C.) to provide esters, which may be separated by those skilled in the art. The ester may be hydrolyzed under standard conditions (e.g. NaOH, DMF/water) to provide the carboxylic acid A4 or A5.

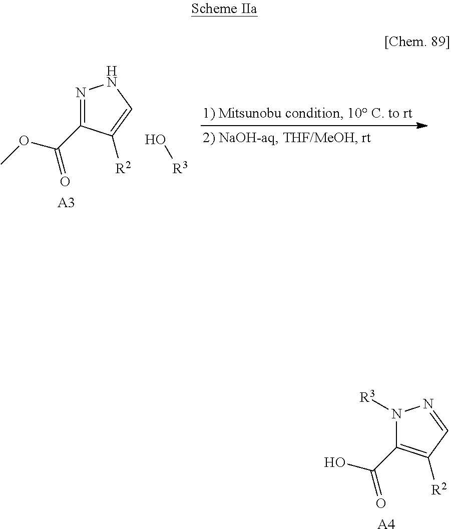

wherein all symbols are defined as below.

Scheme IIa illustrates a general route to the intermediates A4. Intermediate A3 may be alkylated with an alcohol (e.g. R³—OH) under standard Mitsunobu conditions (e.g. PPh₃ and Diisopropyl azodicarboxylate) in solvent (e.g. THF) to provide esters. The ester may be hydrolyzed under standard conditions (e.g. NaOH-aq, THF/MeOH) to provide the carboxylic acid A4.

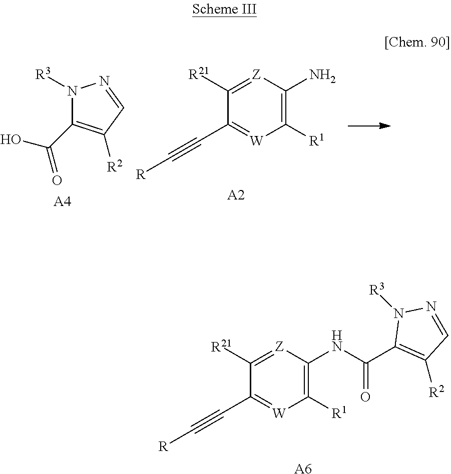

wherein all symbols are defined as below.

Scheme III illustrates a general route to compounds of formula (A6). Compounds A6 may be formed by standard amide coupling conditions (e.g. PyClU, pyridine, DCE) heating to around 60° C.

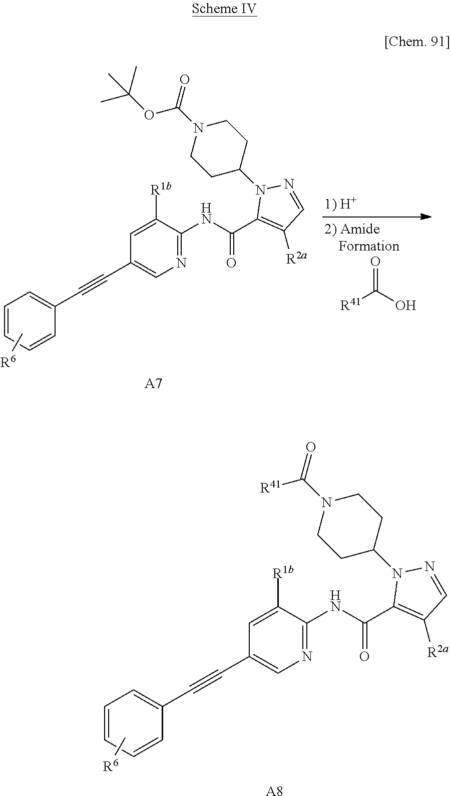

wherein R⁴¹ is selected from (1) C1-C10-alkyl (2) C3-C10-cycloalkyl and (3)₃ to 15 membered heterocycle and the other symbols are defined as below.

As shown in Scheme IV, the intermediate A7 under acidic conditions (e.g. TFA/DCM) may provide the secondary amine that subsequently may be coupled under standard amide coupling conditions (e.g. HATU, DIEA, Carboxylic acid) to form amides A8.

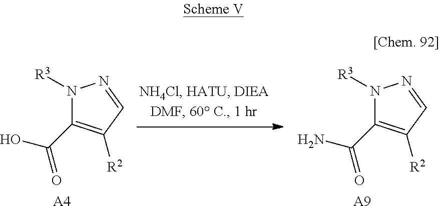

wherein all symbols are defined as below.

Scheme V illustrates a general route to intermediates A9. Compounds A9 may be formed by standard amide formation conditions (e.g. Ammonium Chloride, HATU, DIEA and DMF) and intermediate A4 under heating conditions up to 60° C.

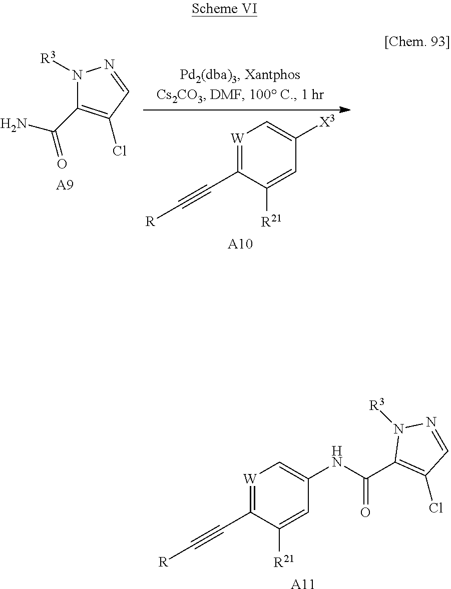

wherein X³ is selected from (1) halogen, (2) tolsylate, (3) mesylate and (4)triflate, and the other symbols are defined as below.

Scheme VI illustrates a general route to Compounds A11. Compounds A11 may be formed by coupling amides A9 with intermediates A10 under standard Palladium cross coupling conditions (e.g. Pd₂(dba)₃, Xantphos, Cs₂CO₃, DMF, up to 100° C.).

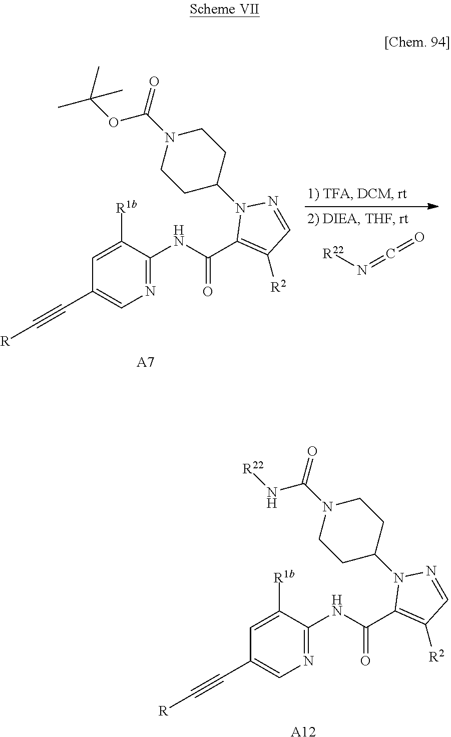

wherein R²² is selected from (1) -C1-C10-alkyl, (2) -C3-C10-cycloalkyl and (3)-3 to 15 membered heterocycle, and the other symbols are defined as below.

As shown in Scheme VII, the intermediate A7 under acidic conditions (e.g. TFA/DCM) may provide the secondary amine that subsequently may be coupled with isonitriles and base (e.g DIEA, THF, rt) to form ureas A12.

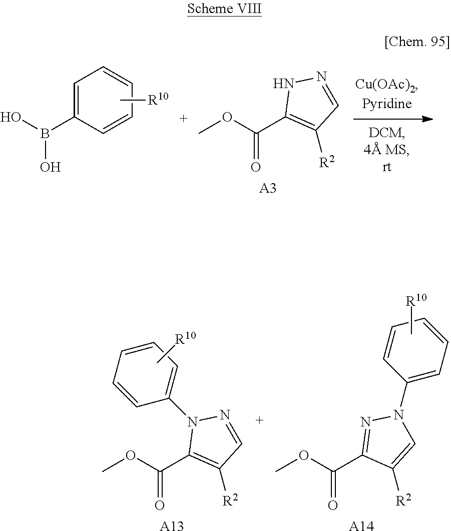

wherein all symbols are defined as below.

Scheme VIII illustrates a general route to aryl esters A13 and A14. Intermediates A13 and A14 may be formed by organometallic cross coupling conditions (e.g. Cu(OAc)₂, pyridine and DCM) in combination with Boronic acids or esters and ester A3.

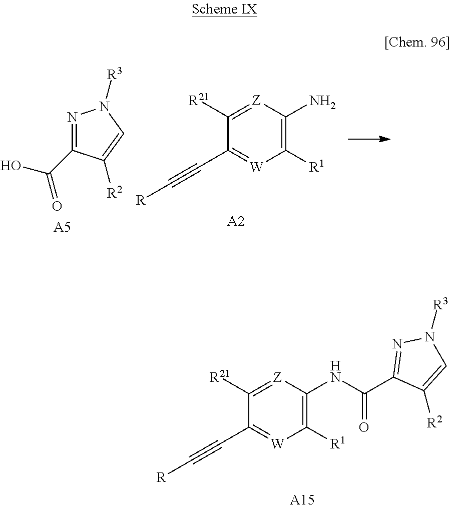

wherein all symbols are defined as below.

Scheme IX illustrates a general route to compounds of formula (A15). Compounds A15 may be formed by standard amide coupling conditions (e.g. PyClU, pyridine, DCE) heating to around 60° C.

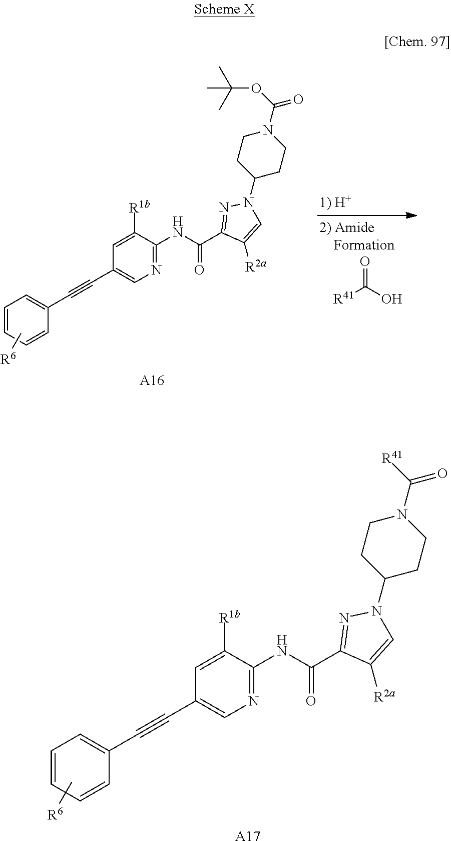

wherein R⁴¹ is C1-C10-alkyl; and the other symbols are defined as below.

As shown in Scheme X, the intermediate A16 under acidic conditions (e.g. TFA/DCM) may provide the secondary amine that subsequently may be coupled under standard amide coupling conditions (e.g. HATU, DIEA, Carboxylic acid) to form amides A17.

The compound substituted with pyrrolidine at the nitrogen atom on the pyrazole can also be synthesized under the same reaction condition as described in Scheme IV, VII or X for the compound with piperidine at the nitrogen atom on the pyrazole.

Compounds of formula (I), specifically bond-pyrazole compounds of A20, can be synthesized as shown in Scheme XI to XIII.

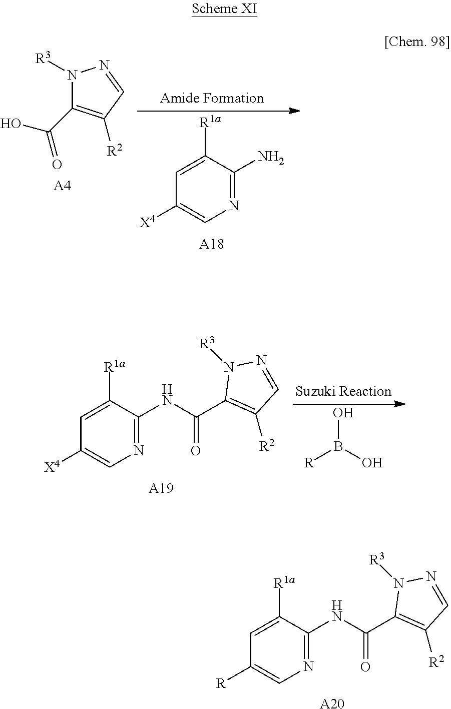

wherein X⁴ is selected from (1) halogen, (2) tolsylate, (3) mesylate and (4)triflate, and the other symbols are defined as below.

As shown in Scheme XI, intermediate A19 may be formed from standard amide coupling conditions (e.g. PyClU, pyridine, DCE) between Intermediate A4 and amine A18. Compounds A20 may be formed under standard Suzuki conditions (e.g. Palladium source, Cs₂CO₃ and dioxane/water).

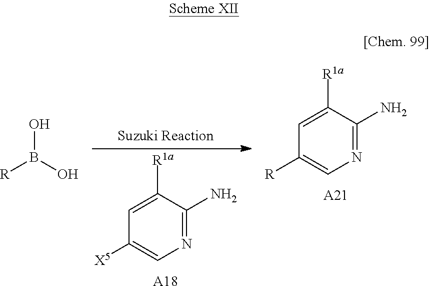

wherein X⁵ is selected from (1) halogen, (2) tosylate, (3) mesylate and (4)triflate, and the other symbols are defined as below.

Compounds A21 may be formed under standard Suzuki conditions (e.g. Palladium source, Cs₂CO₃ and dioxane/water).

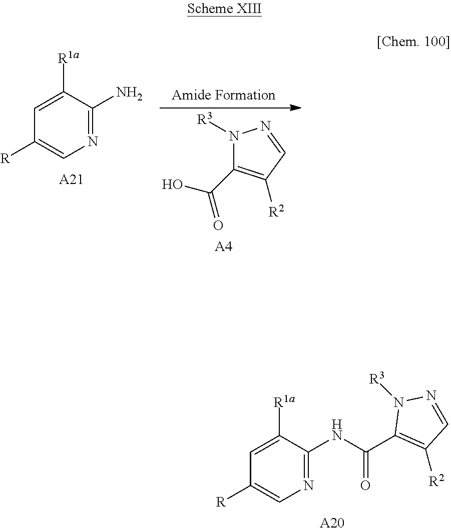

wherein all symbols are defined as below.

Scheme XIII illustrates an alternative route to compounds of formula (A20). Compounds A20 may be formed by standard amide coupling conditions (e.g. PyClU, pyridine, DCE) heating to around 60° C. with amine A21 and carboxylic acid A4.

Compounds of formula (I), specifically —CH₂O-pyrazole compounds of A23, can be synthesized as shown in Scheme XIV.

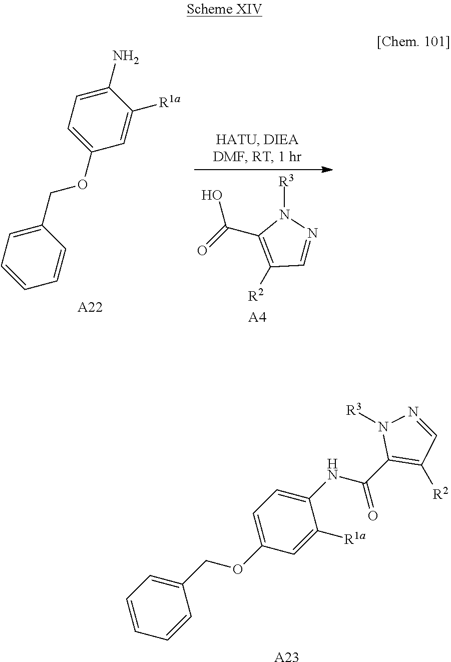

wherein all symbols are defined as below.

As shown in Scheme XIV, compounds A23 may be formed by standard amide coupling conditions (e.g. HATU, DIEA, DMF) with amine A22 and carboxylic acid A4.

Compounds of formula (I), specifically ethynylene-aminopyrazole compounds of A27, can be synthesized as shown in Scheme XV to XVI.

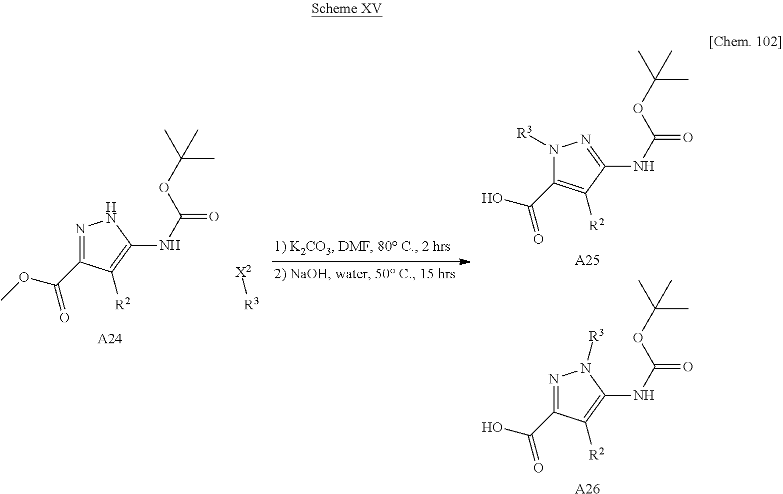

wherein X² is selected from (1) halogen, (2)tosylate, (3) mesylate and (4)triflate and the other symbols are defined as below.

Scheme XV illustrates a general route to the intermediates A25 and regioisomer A26. Intermediate A24 may be alkylated with appropriate coupling partner (e.g. R³—X², where X² is halogen, tosylate, mesylate or triflate), a base (e.g. K₂CO₃) in solvent (e.g. DMF) under heat (e.g. around 100° C.) to provide esters, which may be separated by those skilled in the art. The ester may be hydrolyzed under standard conditions (e.g. NaOH, DMF/water) to provide the carboxylic acid A25 or A26.

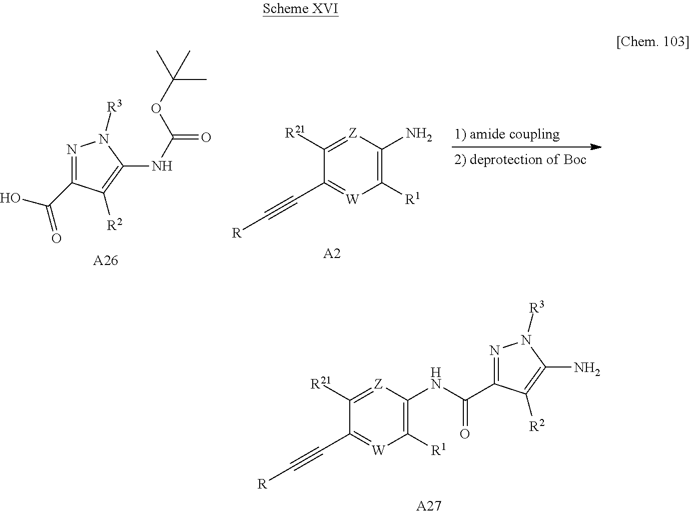

wherein all symbols are defined as below.

As shown in Scheme XVI, compound A27 may be formed from standard amide coupling conditions (e.g. PyClU, pyridine, DCE) between Intermediate A26 and amine A2, followed by standard deprotection condition of Boc group under acidic conditions (e.g. TFA/DCM).

The compounds used as the starting material in each of the reactions is known or can be easily prepared by known method.

The compounds and intermediates may be isolated and purified by methods well-known to those skilled in the art of organic synthesis. Examples of conventional methods for isolating and purifying compounds can include, but are not limited to, chromatography on solid supports such as silica gel, alumina, or silica derivatized with alkylsilane groups, by recrystallization at high or low temperature with an optional pretreatment with activated carbon, thin-layer chromatography, distillation at various pressures, sublimation under vacuum, and trituration, as described for instance in “Vogel's Textbook of Practical Organic Chemistry”, 5th edition (1989), by Furniss, Hannaford, Smith, and Tatchell, pub. Longman Scientific & Technical, Essex CM20 2JE, England.

A disclosed compound may have at least one basic nitrogen whereby the compound can be treated with an acid to form a desired salt. For example, a compound may be reacted with an acid at or above room temperature to provide the desired salt, which is deposited, and collected by filtration after cooling. Examples of acids suitable for the reaction include, but are not limited to tartaric acid, lactic acid, succinic acid, as well as mandelic, atrolactic, methanesulfonic, ethanesulfonic, toluenesulfonic, naphthalenesulfonic, benzenesulfonic, carbonic, fumaric, maleic, gluconic, acetic, propionic, salicylic, hydrochloric, hydrobromic, phosphoric, sulfuric, citric, hydroxybutyric, camphorsulfonic, malic, phenylacetic, aspartic, or glutamic acid, and the like.

Reaction conditions and reaction times for each individual step can vary depending on the particular reactants employed and substituents present in the reactants used. Specific procedures are provided in the Examples section. Reactions can be worked up in the conventional manner, e.g. by eliminating the solvent from the residue and further purified according to methodologies generally known in the art such as, but not limited to, crystallization, distillation, extraction, trituration and chromatography. Unless otherwise described, the starting materials and reagents are either commercially available or can be prepared by one skilled in the art from commercially available materials using methods described in the chemical literature. Starting materials, if not commercially available, can be prepared by procedures selected from standard organic chemical techniques, techniques that are analogous to the synthesis of known, structurally similar compounds, or techniques that are analogous to the above described schemes or the procedures described in the synthetic examples section.

Routine experimentations, including appropriate manipulation of the reaction conditions, reagents and sequence of the synthetic route, protection of any chemical functionality that cannot be compatible with the reaction conditions, and deprotection at a suitable point in the reaction sequence of the method are included in the scope of the invention. Suitable protecting groups and the methods for protecting and deprotecting different substituents using such suitable protecting groups are well known to those skilled in the art; examples of which can be found in PGM Wuts and TW Greene, in Greene's book titled Protective Groups in Organic Synthesis (4^{th} ed.), John Wiley & Sons, NY (2006), which is incorporated herein by reference in its entirety. Synthesis of the compounds of the invention can be accomplished by methods analogous to those described in the synthetic schemes described hereinabove and in specific examples.

When an optically active form of a disclosed compound is required, it can be obtained by carrying out one of the procedures described herein using an optically active starting material (prepared, for example, by asymmetric induction of a suitable reaction step), or by resolution of a mixture of the stereoisomers of the compound or intermediates using a standard procedure (such as chromatographic separation, recrystallization or enzymatic resolution).

Similarly, when a pure geometric isomer of a compound is required, it can be obtained by carrying out one of the above procedures using a pure geometric isomer as a starting material, or by resolution of a mixture of the geometric isomers of the compound or intermediates using a standard procedure such as chromatographic separation.

It can be appreciated that the synthetic schemes and specific examples as described are illustrative and are not to be read as limiting the scope of the invention as it is defined in the appended claims. All alternatives, modifications, and equivalents of the synthetic methods and specific examples are included within the scope of the claims.

TREK1 and TREK2 Activator or Inhibitor Activity

Measurement conditions of TREK1 and TREK2 Activator or Inhibitor Activity are as below. The activity data in Examples were measured by Method 1 unless otherwise specified.

TREK1 Thalium flux Assay

Method 1;

CHO-K1 cells stably expressing human TREK-1 were plated in 384-well plates, cultured overnight, loaded with Thallos dye the following day. Test compounds or control compound (tert-butyl (3-((4-(benzyloxy)-2-methylphenyl)carbamoyl)-4-chlorophenyl)carbamate) or 0.3% DMSO (vehicle control) which were prepared on separate plates were added to the cell plates and incubated for 10 min, and then the cells were treated with thallium stimulus buffer to initiate thallium flux. To measure the efficacy and potency of test compounds, the change in fluorescence intensity (ΔRatio) and % inhibition were calculated using the following equations:

ΔRatio=(fluorescence intensity at 25 seconds after thallium addition)/(average of fluorescent intensity before thallium addition)

% inhibition={1−(ΔRatio of test compound−ΔRatio of 10 μM control compound)/(ΔRatio of 0.3% DMSO−ΔRatio of 10 μM control compound)}×100

Method 2;

CHO-K1 cells stably expressing human TREK-1 were plated in 384-well plates on the day of assay, loaded with Thallos dye. Test compounds or control compound (tert-butyl (3-((4-(benzyloxy)-2-methylphenyl)carbamoyl)-4-chlorophenyl)carbamate) or 0.3% DMSO (vehicle control) were added directly to the cell plates and incubated for 10 min, and then treated with thallium stimulus buffer to initiate thallium flux. To measure the efficacy and potency of test compounds, the change in fluorescence intensity (ΔRatio) and % inhibition were calculated using the following equations:

ΔRatio=(fluorescence intensity at 25 seconds after thallium addition)/(average of fluorescent intensity before thallium addition)

% inhibition={1−(ΔRatio of test compound−ΔRatio of 10 μM control compound)/(ΔRatio of 0.3% DMSO−ΔRatio of 10 μM control compound)}×100

TREK-2 Thallium Flux Assay

Method 1;

HEK293 cells stably expressing human TREK-2 were plated in 384-well plates, cultured overnight, loaded with Thallos dye the following day. Test compounds or control compound (tert-butyl (3-((4-(benzyloxy)-2-methylphenyl)carbamoyl)-4-chlorophenyl)carbamate) or 0.3% DMSO (vehicle control) which were prepared on separate plates were added to the cell plates and incubated for 10 min, and then the cells were treated with thallium stimulus buffer to initiate thallium flux.

To measure the efficacy and potency of test compounds, the change in fluorescence intensity (ΔRatio) and % inhibition were calculated using the following equations:

ΔRatio=(fluorescence intensity at 25 seconds after thallium addition)/(average of fluorescent intensity before thallium addition)

% inhibition={1−(ΔRatio of test compound−ΔRatio of 10 μM control compound)/(ΔRatio of 0.3% DMSO−ΔRatio of 10 μM control compound)}×100

Patch Clamp Technique

Method 1:

CHO-K1 cells stably expressing human TREK-1 or HEK293 cells stably expressing human TREK-2 are plated on glass coverslips, and voltage clamped in the whole-cell configuration of the patch clamp technique. Cells were voltage clamped at a holding potential of −80 mV and the stepped to 0 mV for 500 msec. The voltage was subsequently ramped from −120 mV to +80 mV over a 500 msec duration. This step-ramp protocol was repeated every 10 sec. The bathing solution contained the following: 135 mM NaCl, 5 mM KCl, 2 mM CaCl₂, 1 mM MgCl₂, 5 mM D-Glucose, 10 mM HEPES, 10 mM sucrose (adjusted to pH 7.4 with NaOH, 300 mosmol/kg H₂O). The pipette solution contained the following: 135 mM KCl, 2 mM MgCl₂, 1 mM EGTA, 10 mM HEPES, 2 mM Na₂ATP (adjusted to pH 7.35 with KOH, 285 mosmol/kg H₂O). Test compounds were dissolved into the bathing solution. Experiments on TREK-2 were terminated with the addition of the control compound (tert-butyl (3-((4-(benzyloxy)-2-methylphenyl)carbamoyl)-4-chlorophenyl)carbamate) so that maximal inhibition could be determined. The effects of test compound on the currents were calculated at 0 mV using the following equations:

For CHO cells expressing TREK-1 channel: % inhibition=(1-post current/pre current)×100

For HEK293 cells expressing TREK-2 channel: % inhibition={1−(post current−current in the presence of 10 μM control compound)/(pre current−current in the presence of 10 μM control compound)}×100

In some experiments, the disclosed compounds activate or inhibit TREK1 channel response as an increase or a decrease in thallium fluorescence in thallium flux assay or an increase or a decrease in current measured at 0 mV in patch clamp electrophysiology assays.

The disclosed compounds may activate or inhibit TREK-1 and/or TREK-2 via an activate or an inhibit mechanism or through an allosteric modulation mechanism.

MK-801-Induced Novel Object Recognition Test:

Testing was performed using 6-week-old male Sprague Dawley rats. On the testing day, rats were habituated at the empty box for 10 minutes individually. The disclosed compound was administered orally 1 h prior, followed by administration of MK-801 (0.2 mg/kg, s.c.) 30 min prior to the acquisition trial for 10 min. The retention trial for 10 min was performed with the intertrial interval for 80 min. The rat was allowed to explore the same objects in the acquisition trial. In the retention trial, one of the objects used in the acquisition trial was replaced with a novel one. The exploration time of licking, sniffing or touching was measured in the retention trial. Efficacy of each compound at the dose of 0.3, 1, 3 or 10 mg/kg p.o. (n=12-15) was determined by the recognition index (exploration time to the novel object/total exploration time). The scores of MK-801/vehicle and MK-801/compound-treated groups were statistically analyzed by Dunnett's test.

The disclosed compounds may inhibit TREK1 selectively. The disclosed compounds may inhibit TREK2 selectively. The disclosed compounds may inhibit both TREK1 and TREK2 to varying degrees. The disclosed compounds may inhibit TREK1 and/or TREK2 via a competitive antagonist mechanism or through an allosteric, non-competitive mechanism.

The disclosed compounds may inhibit TREK1 and/or TREK2 response in TREK1 or TREK2-transfected CHO-K1 cells with an IC₅₀ less than, or equivalent to the IC₅₀ for TREK1 or TREK2. That is, a disclosed compound can have selectivity for the TREK1 vis-a-vis TREK2, a disclosed compound can have selectivity for the TREK2 vis-a-vis TREK1, or no selectivity. For example, in some embodiments, a disclosed compound can inhibit TREK1 response with an IC₅₀ of about 5-fold less, about 10-fold less, about 20-fold less, about 30-fold less, about 50-fold less, about 100-fold less, about 200-fold less, about 300-fold less, about 400-fold less, or greater than about 500-fold less than that for TREK2. In some embodiments, a disclosed compound can inhibit TREK2 response with an IC₅₀ of about 5-fold less, about 10-fold less, about 20-fold less, about 30-fold less, about 50-fold less, about 100-fold less, about 200-fold less, about 300-fold less, about 400-fold less, or greater than about 500-fold less than that for TREK1. In some embodiments, a disclosed compound can inhibit TREK1 and TREK2 responses with comparable IC₅₀ values.

The disclosed compounds may activate TREK1 selectively. The disclosed compounds may activate TREK2 selectively. The disclosed compounds may activate both TREK1 and TREK2 to varying degrees. The disclosed compounds may activate TREK1 and/or TREK2 via a competitive agonist mechanism or through an allosteric, non-competitive mechanism.

The disclosed compounds may activate TREK1 and/or TREK2 response in TREK1 or TREK2-transfected CHO-K1 cells with an EC₅₀ less than, or equivalent to the EC₅₀ for TREK1 or TREK2. That is, a disclosed compound can have selectivity for the TREK1 vis-a-vis TREK2, a disclosed compound can have selectivity for the TREK2 vis-a-vis TREK1, or no selectivity. For example, in some embodiments, a disclosed compound can activate TREK1 response with an EC₅₀ of about 5-fold less, about 10-fold less, about 20-fold less, about 30-fold less, about 50-fold less, about 100-fold less, about 200-fold less, about 300-fold less, about 400-fold less, or greater than about 500-fold less than that for TREK2. In some embodiments, a disclosed compound can activate TREK2 response with an EC₅₀ of about 5-fold less, about 10-fold less, about 20-fold less, about 30-fold less, about 50-fold less, about 100-fold less, about 200-fold less, about 300-fold less, about 400-fold less, or greater than about 500-fold less than that for TREK1. In some embodiments, a disclosed compound can activate TREK1 and TREK2 responses with comparable EC₅₀ values.

Pharmaceutical Compositions and Formulations

The disclosed compounds may be incorporated into pharmaceutical compositions suitable for administration to a subject (such as a patient, which may be a human or non-human). The disclosed compounds may also be provided as formulations, such as spray-dried dispersion formulations.

The pharmaceutical compositions and formulations may include a “therapeutically effective amount” or a “prophylactically effective amount” of the agent. A “therapeutically effective amount” refers to an amount effective, at dosages and for periods of time necessary, to achieve the desired therapeutic result. A therapeutically effective amount of the composition may be determined by a person skilled in the art and may vary according to factors such as the disease state, age, sex, and weight of the individual, and the ability of the composition to elicit a desired response in the individual. A therapeutically effective amount is also one in which any toxic or detrimental effects of a compound of the invention (e.g., a compound of formula (I)) are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

For example, a therapeutically effective amount of a compound of formula (I), may be about 0.01 mg to about 1000 mg at a time, about 0.05 mg to about 500 mg at a time, about 0.1 mg to about 500 mg at a time, about 0.5 mg to about 300 mg at a time, about 1 mg to about 250 mg at a time, about 5 mg to about 200 mg at a time and about 10 mg to about 150 mg at a time, by oral administration to a patient for once to several times per day, or about 0.01 mg to about 1000 mg at a time, about 0.05 mg to about 500 mg at a time, about 0.1 mg to about 500 mg at a time, about 0.5 mg to about 300 mg at a time, about 1 mg to about 250 mg at a time, about 5 mg to about 200 mg at a time and about 10 mg to about 150 mg at a time, by parenteral administration to a patient, or continuous administration to a patient for 30 minutes to 24 hours per day intravenously. It may be administrated to patients once to several times per day.

Needless to say, as mentioned above, the effective amount to be used vary dependent upon various conditions. Therefore, effective amount lower than the ranges specified above may be sufficient in some cases, and effective amount higher than the ranges specified above are needed in some cases.

The pharmaceutical compositions and formulations may include pharmaceutically acceptable carriers. The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Thus, the compounds and their physiologically acceptable salts may be formulated for administration by, for example, solid dosing, eye drop, in a topical oil-based formulation, injection, inhalation (either through the mouth or the nose), implants, or oral, buccal, parenteral, or rectal administration. Techniques and formulations may generally be found in “Remington's Pharmaceutical Sciences,” (Meade Publishing Co., Easton, Pa.). Therapeutic compositions must typically be sterile and stable under the conditions of manufacture and storage.

The route by which the disclosed compounds are administered and the form of the composition will dictate the type of carrier to be used. The composition may be in a variety of forms, suitable, for example, for systemic administration (e.g., oral, rectal, nasal, sublingual, buccal, implants, or parenteral) or topical administration (e.g., dermal, pulmonary, nasal, aural, ocular, liposome delivery systems, or iontophoresis).

Carriers for systemic administration typically include at least one of diluents, lubricants, binders, disintegrants, colorants, flavors, sweeteners, antioxidants, preservatives, glidants, solvents, suspending agents, wetting agents, surfactants, combinations thereof, and others. All carriers are optional in the compositions.

Suitable diluents include sugars such as glucose, lactose, dextrose, and sucrose; diols such as propylene glycol; calcium carbonate; sodium carbonate; sugar alcohols, such as glycerin; mannitol; and sorbitol. The amount of diluent(s) in a systemic or topical composition is typically about 50 to about 90%.

Suitable lubricants include silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil and oil of theobroma. The amount of lubricant(s) in a systemic or topical composition is typically about 5 to about 10%.

Suitable binders include polyvinyl pyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose, and sodium carboxymethylcellulose. The amount of binder(s) in a systemic composition is typically about 5 to about 50%.

Suitable disintegrants include agar, alginic acid and the sodium salt thereof, effervescent mixtures, croscarmellose, crospovidone, sodium carboxymethyl starch, sodium starch glycolate, clays, and ion exchange resins. The amount of disintegrant(s) in a systemic or topical composition is typically about 0.1 to about 10%.

Suitable colorants include a colorant such as an FD&C dye. When used, the amount of colorant in a systemic or topical composition is typically about 0.005 to about 0.1%.

Suitable flavors include menthol, peppermint, and fruit flavors. The amount of flavor(s), when used, in a systemic or topical composition is typically about 0.1 to about 1.0%.

Suitable sweeteners include aspartame and saccharin. The amount of sweetener(s) in a systemic or topical composition is typically about 0.001 to about 1%.

Suitable antioxidants include butylated hydroxyanisole (“BHA”), butylated hydroxytoluene (“BHT”), and vitamin E. The amount of antioxidant(s) in a systemic or topical composition is typically about 0.1 to about 5%.

Suitable preservatives include benzalkonium chloride, methyl paraben and sodium benzoate. The amount of preservative(s) in a systemic or topical composition is typically about 0.01 to about 5%.

Suitable glidants include silicon dioxide. The amount of glidant(s) in a systemic or topical composition is typically about 1 to about 5%.

Suitable solvents include water, isotonic saline, ethyl oleate, glycerine, hydroxylated castor oils, alcohols such as ethanol, and phosphate buffer solutions. The amount of solvent(s) in a systemic or topical composition is typically from about 0 to about 100%.

Suitable suspending agents include AVICEL RC-591 (from FMC Corporation of Philadelphia, PA) and sodium alginate. The amount of suspending agent(s) in a systemic or topical composition is typically about 1 to about 8%.

Suitable surfactants include lecithin, Polysorbate 80, and sodium lauryl sulfate, and the TWEENS from Atlas Powder Company of Wilmington, Delaware. Suitable surfactants include those disclosed in the C.T.F.A. Cosmetic Ingredient Handbook, 1992, pp. 587-592; Remington's Pharmaceutical Sciences, 15th Ed. 1975, pp. 335-337; and McCutcheon's Volume 1, Emulsifiers & Detergents, 1994, North American Edition, pp. 236-239. The amount of surfactant(s) in the systemic or topical composition is typically about 0.1% to about 5%.

Although the amounts of components in the systemic compositions may vary depending on the type of systemic composition prepared, in general, systemic compositions include 0.01% to 50% of an active compound (e.g., a compound of formula (I)) and 50% to 99.99% of one or more carriers. Compositions for parenteral administration typically include 0.1% to 10% of actives and 90% to 99.9% of a carrier including a diluent and a solvent.

Compositions for oral administration can have various dosage forms. For example, solid forms include tablets, capsules, granules, and bulk powders. These oral dosage forms include a safe and effective amount, usually at least about 5%, and more particularly from about 25% to about 50% of actives. The oral dosage compositions include about 50% to about 95% of carriers, and more particularly, from about 50% to about 75%.

Tablets can be compressed, tablet triturates, enteric-coated, sugar-coated, filmcoated, or multiple-compressed. Tablets typically include an active component, and a carrier comprising ingredients selected from diluents, lubricants, binders, disintegrants, colorants, flavors, sweeteners, glidants, and combinations thereof. Specific diluents include calcium carbonate, sodium carbonate, mannitol, lactose and cellulose. Specific binders include starch, gelatin, and sucrose. Specific disintegrants include alginic acid and croscarmellose. Specific lubricants include magnesium stearate, stearic acid, and talc. Specific colorants are the FD&C dyes, which can be added for appearance. Chewable tablets preferably contain sweeteners such as aspartame and saccharin, or flavors such as menthol, peppermint, fruit flavors, or a combination thereof.

Capsules (including implants, time release and sustained release formulations) typically include an active compound (e.g., a compound of formula (I)), and a carrier including one or more diluents disclosed above in a capsule comprising gelatin. Granules typically comprise a disclosed compound, and preferably glidants such as silicon dioxide to improve flow characteristics. Implants can be of the biodegradable or the non-biodegradable type.

The selection of ingredients in the carrier for oral compositions depends on secondary considerations like taste, cost, and shelf stability, which are not critical for the purposes of this invention.

Solid compositions may be coated by conventional methods, typically with pH or time-dependent coatings, such that a disclosed compound is released in the gastrointestinal tract in the vicinity of the desired application, or at various points and times to extend the desired action. The coatings typically include one or more components selected from the group consisting of cellulose acetate phthalate, polyvinyl acetate phthalate, hydroxypropyl methyl cellulose phthalate, ethyl cellulose, EUDRAGIT® coatings (available from Evonik Industries of Essen, Germany), waxes and shellac.

Compositions for oral administration can have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid orally administered compositions typically include a disclosed compound and a carrier, namely, a carrier selected from diluents, colorants, flavors, sweeteners, preservatives, solvents, suspending agents, and surfactants. Peroral liquid compositions preferably include one or more ingredients selected from colorants, flavors, and sweeteners.

Other compositions useful for attaining systemic delivery of the subject compounds include sublingual, buccal and nasal dosage forms. Such compositions typically include one or more of soluble filler substances such as diluents including sucrose, sorbitol and mannitol; and binders such as acacia, microcrystalline cellulose, carboxymethyl cellulose, and hydroxypropyl methylcellulose. Such compositions may further include lubricants, colorants, flavors, sweeteners, antioxidants, and glidants.

The disclosed compounds can be topically administered. Topical compositions that can be applied locally to the skin may be in any form including solids, solutions, oils, creams, ointments, gels, lotions, shampoos, leave-on and rinse-out hair conditioners, milks, cleansers, moisturizers, sprays, skin patches, and the like. Topical compositions include: a disclosed compound (e.g., a compound of formula (I)), and a carrier. The carrier of the topical composition preferably aids penetration of the compounds into the skin. The carrier may further include one or more optional components.

The amount of the carrier employed in conjunction with a disclosed compound is sufficient to provide a practical quantity of composition for administration per unit dose of the compound. Techniques and compositions for making dosage forms useful in the methods of this invention are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2nd Ed., (1976).

A carrier may include a single ingredient or a combination of two or more ingredients. In the topical compositions, the carrier includes a topical carrier. Suitable topical carriers include one or more ingredients selected from phosphate buffered saline, isotonic water, deionized water, monofunctional alcohols, symmetrical alcohols, aloe vera gel, allantoin, glycerin, vitamin A and E oils, mineral oil, propylene glycol, PPG-2 myristyl propionate, dimethyl isosorbide, castor oil, combinations thereof, and the like. More particularly, carriers for skin applications include propylene glycol, dimethyl isosorbide, and water, and even more particularly, phosphate buffered saline, isotonic water, deionized water, monofunctional alcohols, and symmetrical alcohols.

The carrier of a topical composition may further include one or more ingredients selected from emollients, propellants, solvents, humectants, thickeners, powders, fragrances, pigments, and preservatives, all of which are optional.

Suitable emollients include stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, din-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate, and combinations thereof. Specific emollients for skin include stearyl alcohol and polydimethylsiloxane. The amount of emollient(s) in a skin-based topical composition is typically about 5% to about 95%.

Suitable propellants include propane, butane, isobutane, dimethyl ether, carbon dioxide, nitrous oxide, and combinations thereof. The amount of propellant(s) in a topical composition is typically about 0% to about 95%.

Suitable solvents include water, ethyl alcohol, methylene chloride, isopropanol, castor oil, ethylene glycol monoethyl ether, diethylene glycol monobutyl ether, diethylene glycol monoethyl ether, dimethylsulfoxide, dimethyl formamide, tetrahydrofuran, and combinations thereof. Specific solvents include ethyl alcohol and homotopic alcohols. The amount of solvent(s) in a topical composition is typically about 0% to about 95%.

Suitable humectants include glycerin, sorbitol, sodium 2-pyrrolidone-5-carboxylate, soluble collagen, dibutyl phthalate, gelatin, and combinations thereof. Specific humectants include glycerin. The amount of humectant(s) in a topical composition is typically 0% to 95%.

The amount of thickener(s) in a topical composition is typically about 0% to about 95%.

Suitable powders include beta-cyclodextrins, hydroxypropyl cyclodextrins, chalk, talc, fullers earth, kaolin, starch, gums, colloidal silicon dioxide, sodium polyacrylate, tetra alkyl ammonium smectites, trialkyl aryl ammonium smectites, chemicallymodified magnesium aluminum silicate, organically-modified montmorillonite clay, hydrated aluminum silicate, fumed silica, carboxyvinyl polymer, sodium carboxymethyl cellulose, ethylene glycol monostearate, and combinations thereof. The amount of powder(s) in a topical composition is typically 0% to 95%.

The amount of fragrance in a topical composition is typically about 0% to about 0.5%, particularly, about 0.001% to about 0.1%.

Suitable pH adjusting additives include HCl or NaOH in amounts sufficient to adjust the pH of a topical pharmaceutical composition.

In some embodiments, the following components are mixed with each other in a usual method and punched out to obtain 10,000 tablets each containing 5 mg of the active ingredient.
1-(cis-4-acetamidocyclohexyl)-4-chloro-N-(3-methyl-5-(phenylethynyl)pyridin-2-yl)-1H-pyrazole-5-carboxamide (50 g);
carboxymethylcellulose calcium (disintegrating agent) (20 g);
magnesium stearate (lubricant) (10 g);
microcrystalline cellulose (920 g).

Spray-Dried Dispersion Formulations

The disclosed compounds may be formulated as a spray-dried dispersion (SDD). An SDD is a single-phase, amorphous molecular dispersion of a drug in a polymer matrix. It is a solid solution with the compound molecularly “dissolved” in a solid matrix. SDDs are obtained by dissolving drug and a polymer in an organic solvent and then spray-drying the solution. The use of spray drying for pharmaceutical applications can result in amorphous dispersions with increased solubility of Biopharmaceutics Classification System (BCS) class II (high permeability, low solubility) and class IV (low permeability, low solubility) drugs. Formulation and process conditions are selected so that the solvent quickly evaporates from the droplets, thus allowing insufficient time for phase separation or crystallization. SDDs have demonstrated long-term stability and manufacturability. For example, shelf lives of more than 2 years have been demonstrated with SDDs. Advantages of SDDs include, but are not limited to, enhanced oral bioavailability of poorly water-soluble compounds, delivery using traditional solid dosage forms (e.g., tablets and capsules), a reproducible, controllable and scalable manufacturing process and broad applicability to structurally diverse insoluble compounds with a wide range of physical properties.

This in one embodiment, the disclosure may provide a spray-dried dispersion formulation comprising a compound of formula (I).

Methods of Use

The disclosed compounds, pharmaceutical compositions and formulations may be used in methods for treatment of disorders, such as neurological, pshychiatric, inflammatory, respiratory, renal and cardiovascular disorders associated with K₂P K⁺ channels, specifically TREK (TWIK RElated K⁺ channels) dysfunction for which activators or inhibitors of TREK1, TREK2 or both TREK1 and TREK2 would offer therapeutic benefit.

Treating Disorders

The disclosed compounds, pharmaceutical compositions and formulations may be used in methods for prevention and/or treatment of disorders, such as neurological and/or psychiatric disorders, associated with TREK channel dysfunction in which inhibitors of TREK1, TREK2 or both TREK1 and TREK2 would offer therapeutic benefit. The methods of prevention and/or treatment may comprise administering to a subject in need of such prevention and/or treatment a therapeutically effective amount of the compound of formula (Ia), or a pharmaceutical composition comprising a therapeutically effective amount of a compound of formula (Ia).

In some embodiments, the disclosure provides to a method for enhancing cognition and/or treating, preventing, ameliorating, controlling or reducing the risk of psychiatric symptoms such as schizophrenia and depression in a mammal comprising the step of administering to the mammal a therapeutically effective amount of the compound of formula (Ia), or a pharmaceutical composition comprising a therapeutically effective amount of a compound of formula (Ia).

The compounds and compositions disclosed herein may be useful for treating, preventing, ameliorating, controlling or reducing the risk of a variety of disorders associated with selective TREK channel inhibition. Thus, provided is a method of treating or preventing a disorder in a subject comprising the step of administering to the subject at least one disclosed compound or at least one disclosed pharmaceutical composition, in an amount effective to treat the disorder in the subject.

Also provided is a method for the prevention and/or treatment of one or more disorders associated with TREK channel activity in a subject comprising the step of administering to the subject a therapeutically effective amount of the compound of formula (Ia), or a pharmaceutical composition comprising a therapeutically effective amount of a compound of formula (Ia).

In some embodiments, the disclosure provides a method for the prevention and/or treatment of a disorder associated with TREK channel dysfunction in which inhibitors of TREK1, TREK2 or both TREK1 and TREK2 would offer therapeutic benefit in a mammal, comprising the step of administering to the mammal an effective amount of at least one disclosed compound or a pharmaceutically acceptable salt thereof, or a pharmaceutical composition comprising at least one disclosed compound or pharmaceutically acceptable salt thereof.

In some embodiments, the disclosed compounds and compositions activating TREK1 and/or 2 have utility in preventing and/or treating pain, migraine, nasal inflammation, atrial fibrillation, acute respiratory distress syndrome, acute lung injury, overactive bladder, cerebral ischemia, epilepsy, amyotrophic lateral sclerosis, neuronal degenerative diseases (e.g. Alzheimer's disease), sepsis, pancreatic cancer, Cushing's syndrome, autosomal dominant polycystic kidney disease, bone fracture, osteoporosis, temporal lobe epilepsy, schizophrenia or colitis or addiction.

In some embodiments, the disclosed compounds and compositions have utility in preventing and/or treating a variety of neurological, psychiatric and cognitive disorders or cancer associated with the TREK-1 and/or 2 inhibition in which inhibitors of TREK1, TREK2 or both TREK1 and TREK2 would offer therapeutic benefit, including one or more of the following conditions or diseases: schizophrenia, psychotic disorder NOS, brief psychotic disorder, schizophreniform disorder, schizoaffective disorder, delusional disorder, shared psychotic disorder, catastrophic schizophrenia, postpartum psychosis, psychotic depression, psychotic break, tardive psychosis, myxedematous psychosis, occupational psychosis, menstrual psychosis, secondary psychotic disorder, bipolar I disorder with psychotic features, substance-induced psychotic disorder, neuropathic pain, prostatic and ovarian cancer. In some embodiments, the psychotic disorder is a psychosis associated with an illness selected from major depressive disorder, postpartum depression, treatment-resistant depression, affective disorder, bipolar disorder, electrolyte disorder, Alzheimer's disease, neurological disorder, hypoglycemia, AIDS, lupus, and post-traumatic stress disorder and 22q11.2 deletion disorder.

In some embodiments, the neurological disorder is selected from brain tumor, dementia with Lewy bodies, cerebrovascular dementia, multiple sclerosis, sarcoidosis, Lyme disease, syphilis, Alzheimer's disease, Parkinson's disease, and anti-NMDA receptor encephalitis.

In some embodiments, the psychotic disorder is selected from schizophrenia, brief psychotic disorder, schizophreniform disorder, schizoaffective disorder, delusional disorder, and shared psychotic disorder. In some embodiments, the schizophrenia is selected from catastrophic schizophrenia, catatonic schizophrenia, paranoid schizophrenia, residual schizophrenia, disorganized schizophrenia, and undifferentiated schizophrenia. In some embodiments, the disorder is selected from schizoid personality disorder, schizotypal personality disorder, and paranoid personality disorder. In some embodiments, the psychotic disorder is due to a general medical condition and is substance-induced or drug-induced (phencyclidine, ketamine and other dissociative anesthetics, amphetamine and other psychostimulants, and cocaine).

In some embodiments, schizophrenia, brief psychotic disorder, schizophreniform disorder, schizoaffective disorder, delusional disorder, and shared psychotic disorder are preferred for the neurological disorder.

The term “schizophrenia” used herein includes negative symptoms of schizophrenia and cognitive impairment associated with schizophrenia (CIAS).

In some embodiments, the present disclosure provides a method for preventing and/or treating a cognitive disorder, comprising administering to a patient in need thereof an effective amount of a compound or a composition of the present disclosure. In some embodiments, cognitive disorders include dementia (associated with Alzheimer's disease, ischemia, multi-infarct dementia, trauma, vascular problems or stroke, HIV disease, Parkinson's disease, Huntington's disease, Pick's disease, Creutzfeldt-Jacob disease, perinatal hypoxia, other general medical conditions or substance abuse), delirium, amnestic disorder, substance-induced persisting delirium, dementia due to HIV disease, dementia due to Huntington's disease, dementia due to Parkinson's disease, Parkinsonian-ALS demential complex, dementia of the Alzheimer's type, age-related cognitive decline, and mild cognitive impairment.

The text revision of the fourth edition of the Diagnostic and Statistical Manual of Mental Disorders (DSM-IV-TR) (2000, American Psychiatric Association, Washington DC) provides a diagnostic tool that includes cognitive disorders including dementia, delirium, amnestic disorders and age-related cognitive decline. The fifth edition of the Diagnostic and Statistical Manual of Mental Disorders (DSM-5) (2013, American Psychiatric Association, Washington DC) provides a diagnostic tool for neurocognitive disorders (NCDs) that include delirium, followed by the syndromes of major NCD, mild NCD, and their etiological subtypes. The major or mild NCD subtypes include NCD due to Alzheimer's disease, vascular NCD, NCD with Lewy bodies, NCD due to Parkinson's disease, frontotemporal NCD, NCD due to traumatic brain injury, NCD due to HIV infection, substance/medication-induced NCD, NCD due to Huntington's disease, NCD due to prion disease, NCD due to another medical condition, NCD due to multiple etiologies, and unspecified NCD. The NCD category in DSM-5 encompasses the group of disorders in which the primary clinical deficit is in cognitive function, and that are acquired rather than developmental. As used herein, the term “cognitive disorders” includes prevention and/or treatment of those cognitive disorders and neurocognitive disorders as described in DSM-IV-TR or DSM-5. The skilled artisan will recognize that there are alternative nomenclatures, nosologies and classification systems for mental disorders, and that these systems evolve with medical and scientific progress. Thus the term “cognitive disorders” is intended to include like disorders that are described in other diagnostic sources.

In some embodiments, the present disclosure provides a method for preventing and/or treating schizophrenia or psychosis, comprising administering to a patient in need thereof an effective amount of a compound or composition of the present disclosure. Particular schizophrenia or psychosis pathologies are paranoid, disorganized, catatonic or undifferentiated schizophrenia and substance-induced psychotic disorder. DSM-IV-TR provides a diagnostic tool that includes paranoid, disorganized, catatonic, undifferentiated or residual schizophrenia, and substance-induced psychotic disorder. DSM-5 eliminated the subtypes of schizophrenia, and instead includes a dimensional approach to rating severity for the core symptoms of schizophrenia, to capture the heterogeneity in symptom type and severity expressed across individuals with psychotic disorders. As used herein, the term “schizophrenia or psychosis” includes prevention and/or treatment of those mental disorders as described in DSM-IV-TR or DSM-5. The skilled artisan will recognize that there are alternative nomenclatures, nosologies and classification systems for mental disorders, and that these systems evolve with medical and scientific progress. Thus the term “schizophrenia or psychosis” is intended to include like disorders that are described in other diagnostic sources.

In some embodiments, the present disclosure provides a method for preventing and/or treating pain, comprising administering to a patient in need thereof an effective amount of a compound or composition of the present disclosure. Particular pain embodiments are bone and joint pain (osteoarthritis), repetitive motion pain, dental pain, cancer pain, myofascial pain (muscular injury, fibromyalgia), perioperative pain (general surgery, gynecological), chronic pain and neuropathic pain.

The compounds and compositions may be further useful in a method for the prevention, treatment, control, amelioration, or reduction of risk of the diseases, disorders and conditions noted herein. The compounds and compositions may be further useful in a method for the prevention, treatment, control, amelioration, or reduction of risk of the aforementioned diseases, disorders and conditions, in combination with other agents.

In the prevention and/or treatment of conditions which require inhibition of a TREK channel (TREK1, TREK2 or dual TREK1/2), an appropriate dosage level may be about 0.01 to 500 mg per day, which can be administered to a patient in single or multiple doses. The dosage level may be about 1 to about 300 mg per day, or about 5 to about 200 mg per day, which can be administered to a patient in single or multiple doses. A suitable dosage level can be about 1 to 250 mg per day, about 5 to 200 mg per day, or about 10 to 150 mg per day, which can be administered to a patient in single or multiple doses. Within this range the dosage can be 0.05 to 0.5, 0.5 to 5 or 5 to 50 mg per day, which can be administered to a patient in single or multiple doses. For oral administration, the compositions may be provided in the form of tablets containing 1.0 to 500 milligrams of the active ingredient, particularly 1.0, 5.0, 10, 15, 20, 25, 50, 75, 100, 150, 200, 250, 300, 400 or 500 milligrams of the active ingredient for the symptomatic adjustment of the dosage to the patient to be treated. The compounds can be administered on a regimen of 1 to 4 times per day, preferably once or twice per day. This dosage regimen can be adjusted to provide the optimal therapeutic response. It will be understood, however, that the specific dose level and frequency of dosage for any particular patient can be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

In some embodiments, the disorder in which inhibitors of TREK1, TREK2 or both TREK1 and TREK2 would offer therapeutic benefit can be selected from psychosis, schizophrenia, conduct disorder, disruptive behavior disorder, bipolar disorder, psychotic episodes of anxiety, anxiety associated with psychosis, psychotic mood disorders such as severe major depressive disorder; mood disorders associated with psychotic disorders, acute mania, depression associated with bipolar disorder, mood disorders associated with schizophrenia, behavioral manifestations of mental retardation, autistic disorder, movement disorders, Tourette's syndrome, akinetic-rigid syndrome, movement disorders associated with Parkinson's disease, tardive dyskinesia, drug induced and neurodegeneration based dyskinesias, attention deficit hyperactivity disorder, cognitive disorders, dementias, and memory disorders.

In some embodiments, the disorder in which inhibitors of TREK1, TREK2 or both TREK1 and TREK2 would offer therapeutic benefit is Alzheimer's disease.

The disclosed compounds, pharmaceutical compositions and formulations may be used in methods for prevention and/or treatment of disorders associated with TREK channel dysfunction in which activators of TREK1, TREK2 or both TREK1 and TREK2 would offer therapeutic benefit. The methods of prevention and/or treatment may comprise administering to a subject in need of such prevention and/or treatment a therapeutically effective amount of the compound of formula (Ib), or a pharmaceutical composition comprising a therapeutically effective amount of a compound of formula (Ib).

In some embodiments, the disclosure provides to a method for enhancing cognition in a mammal comprising the step of administering to the mammal a therapeutically effective amount of the compound of formula (Ib), or a pharmaceutical composition comprising a therapeutically effective amount of a compound of formula (Ib).

The compounds and compositions disclosed herein may be useful for treating, preventing, ameliorating, controlling or reducing the risk of a variety of disorders associated with TREK channel dysfunction in which activators of TREK1, TREK2 or both TREK1 and TREK2 would offer therapeutic benefit. Thus, provided is a method of preventing and/or treating a disorder in a subject comprising the step of administering to the subject at least one disclosed compound or at least one disclosed pharmaceutical composition, in an amount effective to prevent and/or treat the disorder in the subject.

Also provided is a method for the prevention and/or treatment of one or more disorders associated with TREK channel dysfunction in a subject comprising the step of administering to the subject a therapeutically effective amount of the compound of formula (Ib), or a pharmaceutical composition comprising a therapeutically effective amount of a compound of formula (Ib).

In some embodiments, the disclosure provides a method for the prevention and/or treatment of a disorder associated with TREK channel dysfunction in which activators of TREK1, TREK2 or both TREK1 and TREK2 would offer therapeutic benefit in a mammal, comprising the step of administering to the mammal an effective amount of at least one disclosed compound or a pharmaceutically acceptable salt thereof, or a pharmaceutical composition comprising at least one disclosed compound or pharmaceutically acceptable salt thereof.

In the prevention and/or treatment of conditions which require activation of a TREK channel (TREK-1, TREK-2 or dual TREK-1 and 2), an appropriate dosage level may be about 0.01 to 500 mg per day, which can be administered to a patient in single or multiple doses. The dosage level may be about 1 to about 300 mg per day, or about 5 to about 200 mg per day, which can be administered to a patient in single or multiple doses. A suitable dosage level can be about 1 to 250 mg per day, about 5 to 200 mg per day, or about 10 to 150 mg per day, which can be administered to a patient in single or multiple doses. Within this range the dosage can be 0.05 to 0.5, 0.5 to 5 or 5 to 50 mg per day, which can be administered to a patient in single or multiple doses. For oral administration, the compositions may be provided in the form of tablets containing 1.0 to 500 milligrams of the active ingredient, particularly 1.0, 5.0, 10, 15, 20, 25, 50, 75, 100, 150, 200, 250, 300, 400 or 500 milligrams of the active ingredient for the symptomatic adjustment of the dosage to the patient to be treated. The compounds can be administered on a regimen of 1 to 4 times per day, preferably once or twice per day. This dosage regimen can be adjusted to provide the optimal therapeutic response. It will be understood, however, that the specific dose level and frequency of dosage for any particular patient can be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

In some embodiments, the disorder in which activators of TREK1, TREK2 or both TREK1 and TREK2 would offer therapeutic benefit can be selected from pain, migraine, nasal inflammation, atrial fibrillation, acute respiratory distress syndrome, acute lung injury, overactive bladder, cerebral ischemia, epilepsy, amyotrophic lateral sclerosis, neuronal degenerative diseases (e.g. Alzheimer's disease), sepsis, pancreatic cancer, Cushing's syndrome, autosomal dominant polycystic kidney disease, bone fracture, osteoporosis, temporal lobe epilepsy, schizophrenia, colitis, or addiction.

Co-Therapeutic Methods

The present invention is further directed to administration of a selective TREK channel inhibitor for improving treatment outcomes in the context of cognitive or behavioral therapy. That is, in some embodiments, the invention relates to a co-therapeutic method comprising a step of administering to a mammal an effective amount and dosage of at least one disclosed compound, or a pharmaceutically acceptable salt thereof.

In some embodiments, administration improves treatment outcomes in the context of cognitive or behavioral therapy. Administration in connection with cognitive or behavioral therapy can be continuous or intermittent. Administration need not be simultaneous with therapy and can be before, during, and/or after therapy. For example, cognitive or behavioral therapy can be provided within 1, 2, 3, 4, 5, 6, 7 days before or after administration of the compound. As a further example, cognitive or behavioral therapy can be provided within 1, 2, 3, or 4 weeks before or after administration of the compound. As a still further example, cognitive or behavioral therapy can be provided before or after administration within a period of time of 1, 2, 3, 4, 5, 6, 7, 8, 9, or 10 half-lives of the administered compound.

It is understood that the disclosed cotherapeutic methods can be used in connection with the disclosed compounds, compositions, kits, and uses.

Combination Therapies

In the methods of use described herein, additional therapeutic agent(s) may be administered simultaneously or sequentially with the disclosed compounds and compositions. Sequential administration includes administration before or after the disclosed compounds and compositions. In some embodiments, the additional therapeutic agent or agents may be administered in the same composition as the disclosed compounds. In other embodiments, there may be an interval of time between administration of the additional therapeutic agent and the disclosed compounds. In some embodiments, administration of an additional therapeutic agent with a disclosed compound may allow lower doses of the other therapeutic agents and/or administration at less frequent intervals. When used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound of Formula (I). The above combinations include combinations of a compound of the present invention not only with one other active compound, but also with two or more other active compounds.

The disclosed compounds can be used as single agents or in combination with one or more other drugs in the treatment, prevention, control, amelioration or reduction of risk of the aforementioned diseases, disorders and conditions for which the compound or the other drugs have utility, where the combination of drugs together are safer or more effective than either drug alone. The other drug(s) can be administered by a route and in an amount commonly used therefor, contemporaneously or sequentially with a disclosed compound. When a disclosed compound is used contemporaneously with one or more other drugs, a pharmaceutical composition in unit dosage form containing such drugs and the disclosed compound may be used. However, the combination therapy can also be administered on overlapping schedules. It is also envisioned that the combination of one or more active ingredients and a disclosed compound can be more efficacious than either as a single agent. Thus, when used in combination with one or more other active ingredients, the disclosed compounds and the other active ingredients can be used in lower doses than when each is used singly.

The pharmaceutical compositions and methods of the present invention can further comprise other therapeutically active compounds as noted herein which are usually applied in the prevention and/or treatment of the above mentioned pathological conditions.

The above combinations include combinations of a disclosed compound not only with one other active compound, but also with two or more other active compounds. Likewise, disclosed compounds can be used in combination with other drugs that are used in the prevention, treatment, control, amelioration, or reduction of risk of the diseases or conditions for which disclosed compounds are useful. Such other drugs can be administered, by a route and in an amount commonly used therefor, contemporaneously or sequentially with a compound of the present invention. When a compound of the present invention is used contemporaneously with one or more other drugs, a pharmaceutical composition containing such other drugs in addition to a disclosed compound is preferred. Accordingly, the pharmaceutical compositions include those that also contain one or more other active ingredients, in addition to a compound of the present invention.

The weight ratio of a disclosed compound to the second active ingredient can be varied and will depend upon the effective dose of each ingredient. Generally, an effective dose of each will be used. Thus, for example, when a compound of the present invention is combined with another agent, the weight ratio of a disclosed compound to the other agent will generally range from about 1000:1 to about 1:1000, preferably about 200:1 to about 1:200. Combinations of a compound of the present invention and other active ingredients will generally also be within the aforementioned range, but in each case, an effective dose of each active ingredient should be used.

In such combinations a disclosed compound and other active agents can be administered separately or in conjunction. In addition, the administration of one element can be prior to, concurrent to, or subsequent to the administration of other agent(s).

Accordingly, the disclosed compounds can be used alone or in combination with other agents which are known to be beneficial in the subject indications or other drugs that affect receptors or enzymes that either increase the efficacy, safety, convenience, or reduce unwanted side effects or toxicity of the disclosed compounds. The subject compound and the other agent can be coadministered, either in concomitant therapy or in a fixed combination.

In some embodiments, the compound can be employed in combination with antiAlzheimer's agents, beta-secretase inhibitors, cholinergic agents, gamma-secretase inhibitors, HMG-CoA reductase inhibitors, M₁ allosteric agonists, M₁ positive allosteric modulators, NSAIDs including ibuprofen, vitamin E, and anti-amyloid antibodies. In another embodiment, the subject compound can be employed in combination with sedatives, hypnotics, anxiolytics, antipsychotics (typical and atypical), anti-anxiety agents, cyclopyrrolones, imidazopyridines, pyrazolopyrimidines, minor tranquilizers, melatonin agonists and antagonists, melatonergic agents, benzodiazepines, barbiturates, 5HT-2 antagonists, and the like, such as: adinazolam, allobarbital, alonimid, alprazolam, amisulpride, amitriptyline, amobarbital, amoxapine, aripiprazole, bentazepam, benzoctamine, brotizolam, bupropion, busprione, butabarbital, butalbital, capuride, carbocloral, chloral betaine, chloral hydrate, clomipramine, clonazepam, cloperidone, clorazepate, chlordiazepoxide, clorethate, chlorpromazine, clozapine, cyprazepam, desipramine, dexclamol, diazepam, dichloralphenazone, divalproex, diphenhydramine, doxepin, estazolam, ethchlorvynol, etomidate, fenobam, flunitrazepam, flupentixol, fluphenazine, flurazepam, fluvoxamine, fluoxetine, fosazepam, glutethimide, halazepam, haloperidol, hydroxyzine, imipramine, lithium, lorazepam, lormetazepam, maprotiline, mecloqualone, melatonin, mephobarbital, meprobamate, methaqualone, midaflur, midazolam, nefazodone, nisobamate, nitrazepam, nortriptyline, olanzapine, oxazepam, paraldehyde, paroxetine, pentobarbital, perlapine, perphenazine, phenelzine, phenobarbital, prazepam, promethazine, propofol, protriptyline, quazepam, quetiapine, reclazepam, risperidone, roletamide, secobarbital, sertraline, suproclone, temazepam, thioridazine, thiothixene, tracazolate, tranylcypromaine, trazodone, triazolam, trepipam, tricetamide, triclofos, trifluoperazine, trimetozine, trimipramine, uldazepam, venlafaxine, zaleplon, ziprasidone, zolazepam, zolpidem, and salts thereof, and combinations thereof, and the like, or the subject compound can be administered in conjunction with the use of physical methods such as with light therapy or electrical stimulation.

In some embodiments, the compound can be employed in combination with levodopa (with or without a selective extracerebral decarboxylase inhibitor such as carbidopa or benserazide), anticholinergics such as biperiden (optionally as its hydrochloride or lactate salt) and trihexyphenidyl (benzhexol) hydrochloride, COMT inhibitors such as entacapone, MOA-B inhibitors, antioxidants, A2a adenosine receptor antagonists, cholinergic agonists, NMDA receptor antagonists, serotonin receptor antagonists and dopamine receptor agonists such as alentemol, bromocriptine, fenoldopam, lisuride, naxagolide, pergolide and pramipexole. It will be appreciated that the dopamine agonist can be in the form of a pharmaceutically acceptable salt, for example, alentemol hydrobromide, bromocriptine mesylate, fenoldopam mesylate, naxagolide hydrochloride and pergolide mesylate. Lisuride and pramipexol are commonly used in a non-salt form.

In some embodiments, the compound can be employed in combination with a compound from the phenothiazine, thioxanthene, heterocyclic dibenzazepine, butyrophenone, diphenylbutylpiperidine and indolone classes of neuroleptic agent. Suitable examples of phenothiazines include chlorpromazine, mesoridazine, thioridazine, acetophenazine, fluphenazine, perphenazine and trifluoperazine. Suitable examples of thioxanthenes include chlorprothixene and thiothixene. An example of a dibenzazepine is clozapine. An example of a butyrophenone is haloperidol. An example of a diphenylbutylpiperidine is pimozide. An example of an indolone is molindolone. Other neuroleptic agents include loxapine, sulpiride and risperidone. It will be appreciated that the neuroleptic agents when used in combination with the subject compound can be in the form of a pharmaceutically acceptable salt, for example, chlorpromazine hydrochloride, mesoridazine besylate, thioridazine hydrochloride, acetophenazine maleate, fluphenazine hydrochloride, flurphenazine enathate, fluphenazine decanoate, trifluoperazine hydrochloride, thiothixene hydrochloride, haloperidol decanoate, loxapine succinate and molindone hydrochloride. Perphenazine, chlorprothixene, clozapine, haloperidol, pimozide and risperidone are commonly used in a non-salt form. Thus, the subject compound can be employed in combination with acetophenazine, alentemol, aripiprazole, amisulpride, benzhexol, bromocriptine, biperiden, chlorpromazine, chlorprothixene, clozapine, diazepam, fenoldopam, fluphenazine, haloperidol, levodopa, levodopa with benserazide, levodopa with carbidopa, lisuride, loxapine, mesoridazine, molindolone, naxagolide, olanzapine, pergolide, perphenazine, pimozide, pramipexole, quetiapine, risperidone, sulpiride, tetrabenazine, trihexyphenidyl, thioridazine, thiothixene, trifluoperazine or ziprasidone.

In some embodiments, the compound can be employed in combination with an antidepressant or anti-anxiety agent, including norepinephrine reuptake inhibitors (including tertiary amine tricyclics and secondary amine tricyclics), selective serotonin reuptake inhibitors (SSRIs), monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, α-adrenoreceptor antagonists, neurokinin-1 receptor antagonists, atypical anti-depressants, benzodiazepines, 5-HT1A agonists or antagonists, especially 5-HT1A partial agonists, and corticotropin releasing factor (CRF) antagonists. Specific agents include: amitriptyline, clomipramine, doxepin, imipramine and trimipramine; amoxapine, desipramine, maprotiline, nortriptyline and protriptyline; fluoxetine, fluvoxamine, paroxetine and sertraline; isocarboxazid, phenelzine, tranylcypromine and selegiline; moclobemide: venlafaxine; duloxetine; aprepitant; bupropion, lithium, nefazodone, trazodone and viloxazine; alprazolam, chlordiazepoxide, clonazepam, chlorazepate, diazepam, halazepam, lorazepam, oxazepam and prazepam; buspirone, flesinoxan, gepirone and ipsapirone, and pharmaceutically acceptable salts thereof.

In some embodiments, the compounds can be coadministered with orthosteric muscarinic agonists, muscarinic potentiators, or cholinesterase inhibitors. In some embodiments, the compounds can be coadministered with GlyT1 inhibitors and the like such as, but not limited to: risperidone, clozapine, haloperidol, fluoxetine, prazepam, xanomeline, lithium, phenobarbitol, and salts thereof and combinations thereof.

In some embodiments, the other drugs for the prevention and/or treatment of schizophrenia is at least one drug selected from typical antipsychotics, atypical antipsychotics, and the like.

The typical antipsychotics may include, for example, chlorpromazine, fluphenazine, haloperidol, sulpiride and the like.

The atypical antipsychotics may include, for example, serotonin-dopamine antagonist, multi-acting receptor targeted antipsychotics, dopamine partial agonist and the like.

The serotonin-dopamine antagonist may include, for example, risperidone, perospirone, ziprasidone, blonanserin and the like.

The multi-acting receptor targeted antipsychotics may include, for example, olanzapine, quetiapine, clozapine, lurasidone and the like.

The dopamine partial agonist may include, for example, aripiprazole, cariprazine and the like.

In some embodiments, the other drugs for the prevention and/or treatment of depression is at least one drug selected from benzodiazepine antianxiety drug, thienodiazepine antianxiety drug, non-benzodiazepine antianxiety drug, neurokinin-1 (NK1) antagonist, tricyclic antidepressant, tetracyclic antidepressant, monoamine oxidase (MAO) inhibitor, triazolopyridine antidepressant, serotonin and noradrenaline reuptake inhibitor (SNRI), selective serotonin reuptake inhibitor (SSRI), serotonin reuptake inhibitor, noradrenergic and specific serotonergic antidepressant (NaSSA), noradrenaline and dopamine disinhibition drug (NDDI), selective serotonin reuptake enhancer (SSRE), and the like.

The benzodiazepine antianxiety drug may include, for example, alprazolam, oxazepam, oxazolam, cloxazolam, clorazepate dipotassium, chlordiazepoxide, diazepam, tofisopam, triazolam, prazepam, fludiazepam, flutazolam, flutoprazepam, bromazepam, mexazolam, medazepam, ethyl loflazepate lorazepam and the like.

The thienodiazepine antianxiety drug may include, for example, etizolam, clotiazepam and the like.

The non-benzodiazepine antianxiety drug may include, for example, citric acid tandospirone, hydroxyzine hydrochloride and the like.

The neurokinin-1 (NK1) antagonist may include, for example, aprepitant, fosaprepitant meglumine and the like.

The tricyclic antidepressant may include, for example, amitriptyline hydrochloride, imipramine hydrochloride, clomipramine hydrochloride, dosulepin hydrochloride, nortriptyline hydrochloride, lofepramine hydrochloride, trimipramine maleate, amoxapine and the like.

The tetracyclic antidepressant may include, for example, maprotiline hydrochloride, mianserin hydrochloride, setiptiline maleate and the like.

The monoamine oxidase (MAO) inhibitor may include, for example, safrazine hydrochloride and the like.

The triazolopyridine antidepressant may include, for example, Trazodone Hydrochloride and the like.

The serotonin and noradrenaline reuptake inhibitor (SNRI) may include, for example, milnacipran hydrochloride, venlafaxine hydrochloride, duloxetine hydrochloride and the like.

The selective serotonin reuptake inhibitor (SSRI) may include, for example, fluvoxamine maleate, paroxetine hydrochloride, fluoxetine hydrochloride, citalopram hydrochloride, sertraline hydrochloride, escitalopram oxalate and the like.

The serotonin reuptake inhibitor may include, for example, trazodone hydrochloride and the like.

The noradrenergic and specific serotonergic antidepressant (NaSSA) may include, for example, mirtazapine and the like.

The noradrenaline and dopamine disinhibition drug (NDDI) may include, for example, agomelatine and the like.

The selective serotonin reuptake enhancer (SSRE) may include, for example, tianeptine and the like.

In some embodiments, the other drugs for the prevention and/or treatment of artrial fibrillation is at least one drug selected from β-blockers, digoxin and the like.

In some embodiments, the other drugs for the prevention and/or treatment of pain is at least one drug selected from acetaminophen, a nonsteroid antiinflammatory drug, an opioid, an antidepressant, an antiepileptic agent, an N-methyl-D-aspartate antagonist, a muscle relaxant, an antiarrhythmic agent, a steroid, a bisphosphonate and the like.

The β-blockers may include, for example, alprenolol hydrochloride, bupranolol hydrochloride, bufetolol hydrochloride, oxprenolol hydrochloride, atenolol, bisoprolol fumarate, betaxolol hydrochloride, bevantolol hydrochloride, metoprolol succinate, metoprolol tartrate, acebutolol hydrochloride, celiprolol hydrochloride, nipradilol, tilisolol hydrochloride, nadorol, propranolol hydrochloride, indenolol hydrochloride, carteolol hydrochloride, pindolol, bunitrolol hydrochloride, landiolol hydrochloride, esmolol hydrochloride, arotinolol hydrochloride, carvedilol, timolol maleate and the like.

The antiarrhythmic agent may include, for example, lidocaine, mexiletine and the like.

The nonsteroid antiinflammatory drug may include, for example, sasapyrine, sodium salicylate, aspirin, aspirin formulations such as those containing aspirin-dialuminate, diflunisal, indomethacin, suprofen, ufenamate, dimethylisopropylazulene, bufexamac, felbinac, diclofenac, tolmetin sodium, Clinoril, fenbufen, nabumetone, proglumetacin, indomethacin farnesil, acemetacin, proglumetacin maleate, amfenac sodium, mofezolac, etodolac, ibuprofen, ibuprofen piconol, naproxen, flurbiprofen, flurbiprofen axetil, ketoprofen, fenoprofen calcium, tiaprofen, oxaprozin, pranoprofen, loxoprofen sodium, alminoprofen, zaltoprofen, mefenamic acid, aluminium mefenamate, tolfenamic acid, floctafenine, ketophenylbutazone, oxyphenbutazone, piroxicam, tenoxicam, ampiroxicam, Napageln ointment, epirizole, tiaramide hydrochloride, tinoridine hydrochloride, emorfazone, sulpyrine, Migrenin, Saridon, Sedes G, Amipylo-N, Sorbon, pilin cold remedies, acetaminophen, phenacetin, dimetotiazine mesilate, meloxicam, celecoxib, rofecoxib, valdecoxib, simetride-containing formulations and non-pilin cold remedies and the like.

The opioid may include, for example, codeine, fentanyl, hydromorphone, levorphanol, meperidine, methadone, morphine, oxycodone, oxymorphone, propoxyphene and the like.

The antidepressant may include, for example, tricyclic antidepressants (e.g., imipramine hydrochloride, clomipramine hydrochloride, dosulepin hydrochloride, nortriptyline hydrochloride, lofepramine hydrochloride, trimipramine maleate, amoxapine), tetracyclic antidepressants (e.g., maprotiline hydrochloride, mianserin hydrochloride, setiptiline maleate), monoamine oxidase (MAO) inhibitors (safrazine hydrochloride), serotonin and noradrenaline reuptake inhibitors (SNRIs) (e.g., milnacipran hydrochloride, venlafaxine hydrochloride), selective serotonin reuptake inhibitors (SSRIs) (e.g., fluvoxamine maleate, paroxetine hydrochloride,), serotonin reuptake inhibitors (e.g., trazodone hydrochloride) and the like.

The antiepileptic agent may include, for example, phenobarbital, Puridomin, phenytoin, ethosuximide, zonisamide, nitrazepam, clonazepam, carbamazepine, sodium valproate, acetazolamide, sulthiame, gabapentin, pregabalin and the like.

The N-methyl-D-aspartate antagonist may include, for example, ketamine hydrochloride, amantadine hydrochloride, memantine hydrochloride, dextromethorphan, methadone and the like.

The muscle relaxant may include, for example, succinylcholine, suxamethonium, vecuronium bromide, pancronium bromide, dantrolene sodium and the like.

The steroid may include, for example, as topical agents, clobetasol propionate, diflorasone diacetate, fluocinonide, mometasone furoate, betamethasone dipropionate, betamethasone butyrate propionate, betamethasone valerate, difluprednate, budesonide, diflucortolone valerate, amcinonide, halcinonide, dexamethasone, dexamethasone propionate, dexamethasone valerate, dexamethasone acetate, hydrocortisone acetate, hydrocortisone butyrate, hydrocortisone butyrate propionate, deprodone propionate, prednisolone valerate acetate, fluocinolone acetonide, beclometasone propionate, triamcinolone acetonide, flumetasone pivalate, alclometasone dipropionate, clobetasone butyrate, prednisolone, beclometasone propionate, fludroxycortide and the like.

The bisphosphonate may include, for example, etidronate, pamidronate, alendronate, risedronate, zoledronate, minodronate and the like.

Modes of Administration

Methods of prevention and/or treatment may include any number of modes of administering a disclosed composition. Modes of administration may include tablets, pills, dragees, hard and soft gel capsules, granules, pellets, aqueous, lipid, oily or other solutions, emulsions such as oil-in-water emulsions, liposomes, aqueous or oily suspensions, syrups, elixirs, solid emulsions, solid dispersions or dispersible powders. For the preparation of pharmaceutical compositions for oral administration, the agent may be admixed with commonly known and used adjuvants and excipients such as for example, gum arabic, talcum, starch, sugars (such as, e.g., mannitose, methyl cellulose, lactose), gelatin, surface-active agents, magnesium stearate, aqueous or non-aqueous solvents, paraffin derivatives, cross-linking agents, dispersants, emulsifiers, lubricants, conserving agents, flavoring agents (e.g., ethereal oils), solubility enhancers (e.g., benzyl benzoate or benzyl alcohol) or bioavailability enhancers (e.g. Gelucire™). In the pharmaceutical composition, the agent may also be dispersed in a microparticle, e.g. a nanoparticulate composition.

For parenteral administration, the agent can be dissolved or suspended in a physiologically acceptable diluent, such as, e.g., water, buffer, oils with or without solubilizers, surface-active agents, dispersants or emulsifiers. As oils for example and without limitation, olive oil, peanut oil, cottonseed oil, soybean oil, castor oil and sesame oil may be used. More generally spoken, for parenteral administration, the agent can be in the form of an aqueous, lipid, oily or other kind of solution or suspension or even administered in the form of liposomes or nano-suspensions.

The term “parenterally,” as used herein, refers to modes of administration which include intravenous, intramuscular, intraperitoneal, intrasternal, subcutaneous and intraarticular injection and infusion.

The present compound has low toxicity and thus can be safely used as a medicament.

Kits

In one aspect, the disclosure provides kits comprising at least one disclosed compound or a pharmaceutically acceptable salt thereof, and one or more of:
(a) at least one agent known to decrease TREK1 activity;
(b) at least one agent known to decrease TREK2 activity;
(c) at least one agent known to prevent and/or treat a disorder associated with TREK dysfunction in which inhibitors of TREK1, TREK2 or both TREK1/TREK2 would offer therapeutic benefit in a mammal;
(d) instructions for preventing and/or treating a disorder associated with TREK dysfunction in which inhibitors of TREK1, TREK2 or both TREK1/TREK2 would offer therapeutic benefit in a mammal; or
(e) instructions for administering the compound in connection with cognitive or behavioral therapy.

In one aspect, the disclosure provides kits comprising at least one disclosed compound or a pharmaceutically acceptable salt thereof, and one or more of:
(a) at least one agent known to increase TREK1 activity;
(b) at least one agent known to increase TREK2 activity;
(c) at least one agent known to prevent and/or treat a disorder associated with TREK activity in which activators of TREK1, TREK2 or both TREK1/TREK2 would offer therapeutic benefit in a mammal; or
(d) instructions for preventing and/or treating a disorder associated with TREK activity.

In some embodiments, the at least one disclosed compound and the at least one agent are co-formulated. In some embodiments, the at least one disclosed compound and the at least one agent are co-packaged. The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

That the disclosed kits can be employed in connection with disclosed methods of use.

The kits may include information, instructions, or both that use of the kit will provide prevention and/or treatment for medical conditions in mammals (particularly humans). The information and instructions may be in the form of words, pictures, or both, and the like. In addition or in the alternative, the kit may include the compound, a composition, or both; and information, instructions, or both, regarding methods of application of compound, or of composition, preferably with the benefit of preventing and/or treating medical conditions in mammals (e.g., humans).

The compounds and processes of the invention will be better understood by reference to the following examples, which are intended as an illustration of and not a limitation upon the scope of the invention.

### EXAMPLES

All NMR spectra were recorded on a 400 MHz AMX Bruker NMR spectrometer. ¹H chemical shifts are reported in o values in ppm downfield with the deuterated solvent as the internal standard. Data are reported as follows: chemical shift, multiplicity (s=singlet, bs=broad singlet, d=doublet, t=triplet, q=quartet, dd=doublet of doublets, m=multiplet, ABq=AB quartet), coupling constant, integration.

LCMS analysis conditions are as below. The MS data in Examples were measured by Reversed-phase LCMS method (1) unless otherwise specified.

Reversed-Phase LCMS (1):

Reversed-phase LCMS analysis was performed using an Agilent 1200 system comprised of a binary pump with degasser, high-performance autosampler, thermostatted column compartment, C18 column, diode-array detector (DAD) and an Agilent 6150 MSD with the following parameters. The gradient conditions were 5% to 95% acetonitrile with the aqueous phase 0.1% TFA in water over 1.4 minutes. Samples were separated on a Waters Acquity UPLC BEH C18 column (1.7 m, 1.0×50 mm) at 0.5 mL/min, with column and solvent temperatures maintained at 55° C. The DAD was set to scan from 190 to 300 nm, and the signals used were 220 nm and 254 nm (both with a band width of 4 nm). The MS detector was configured with an electrospray ionization source, and the low-resolution mass spectra were acquired by scanning from 140 to 700 AMU with a step size of 0.2 AMU at 0.13 cycles/second, and peak width of 0.008 minutes. The drying gas flow was set to 13 liters per minute at 300° C. and the nebulizer pressure was set to 30 psi. The capillary needle voltage was set at 3000 V, and the fragmentor voltage was set at 100V. Data acquisition was performed with Agilent Chemstation and Analytical Studio Reviewer software.

Reversed-Phase LCMS (2)

Reversed-phase LCMS analysis was obtained on a SHIMADZU LC20-MS2010 with ESI source. MS parameters were as follows: Mobile Phase: 1.5 mL/4 L TFA in water (solvent A) and 0.75 mL/4 L TFA in acetonitrile (solvent B), using the elution gradient 5%-95% (solvent B) over 0.7 minutes and holding at 95% for 0.4 minutes at a flow rate of 1.5 ml/min; Column: Agilent Pursit 5 C18 20*2.0 mm; Wavelength: UV 220 nm, 254 nm, 215 nm; Column temperature: 50° C.; MS ionization: ESI.

Reversed-Phase LCMS (3)

Reversed-phase LCMS analysis was obtained on a SHIMADZU LCMS-2020 with ESI source. MS parameters were as follows: Mobile Phase: 0.1% TFA in water (solvent A) and 0.1% TFA in acetonitrile (solvent B), using the elution holding at 5% (solvent B) for 0.1 minutes, gradient 5%-95% (solvent B) over 1.1 minutes and holding at 95% for 0.4 minutes at a flow rate of 1.0 ml/min; Column: YMC Triart C18<D2.0 mm*L30 mm; Wavelength: UV 220 nm, 254 nm; Column temperature: 30° C.; detector MS, ELSD; MS ionization: ESI.

Abbreviations which have been used in the descriptions of following examples are: IPA is isopropyl alcohol; AcOH is acetic acid; BOP is (benzotriazol-1-yloxy)tris(dimethylamino)phosphonium hexafluorophosphate; DBU is 1,8-diazabicyclo(5.4.0)undec-7-ene; DCE is 1,2-Dichloroethane; DCM is dichloromethane; DIEA is N,N-diisopropylethylamine; DMF is N,N-dimethylformamide; DMSO is dimethyl sulfoxide; EtOAc or EA is ethyl acetate; HATU is 1-[bis(dimethylamino)methylene]-1H-1,2,3-triazolo[4,5-b]pyridinium 3-oxid hexafluorophosphate; NMP is N-methyl-2-pyrrolidone; MW is microwave (referring to a microwave reactor); PyClU is 1-(chloro-1-pyrrolidinylmethylene)pyrrolidinium hexafluorophosphate; PE is Petroleum Ether; RBF is round-bottomed flask; and RT or rt is room temperature.

Intermediates:

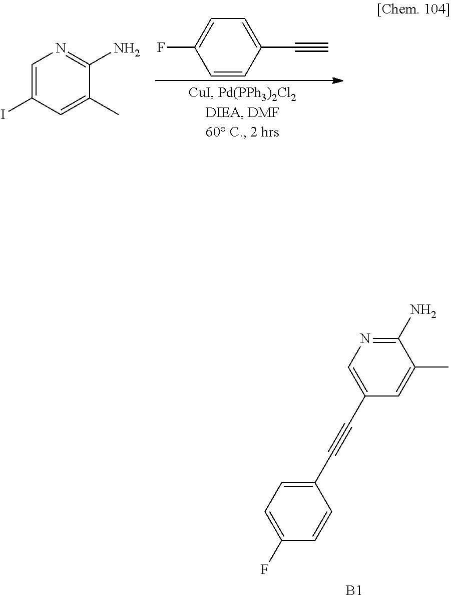

5-((4-fluorophenyl)ethynyl)-3-methylpyridin-2-amine (B1): To a microwave vial was added 5-iodo-2-amino-3-picoline (2000 mg, 8.55 mmol), N,N-diisopropylethylamine (10.42 mL, 59.82 mmol), trans-dichlorobis(triphenylphosphine)palladium(II) (602 mg, 0.85 mmol), copper(I) iodide (163 mg, 0.85 mmol), and 4-fluorophenylacetylene (2.94 mL, 25.64 mmol). The vial was capped, evacuated and purged with N₂ (3×) and then diluted with DMF (2 mL). The mixture was heated to 60° C. for 2 hours in the microwave. After LCMS of the reaction confirmed product, the sample was filtered through a pad of celite and rinsed with (3:1) CHCl₃:IPA. The sample was concentrated and then purified by normal phase column chromatography (gradient: 0-80% EtOAc in Hexanes) to yield (B1) (1564 mg, 81% yield). ES-MS [M+1]+: 227.2, ¹H NMR (400 MHz, CDCl₃) δ 8.15 (d, J=1.3 Hz, 1H), 7.49-7.42 (m, 2H), 7.41 (d, J=0.7 Hz, 1H), 7.03 (m, 2H), 4.58 (s, 2H), 2.13 (s, 3H).

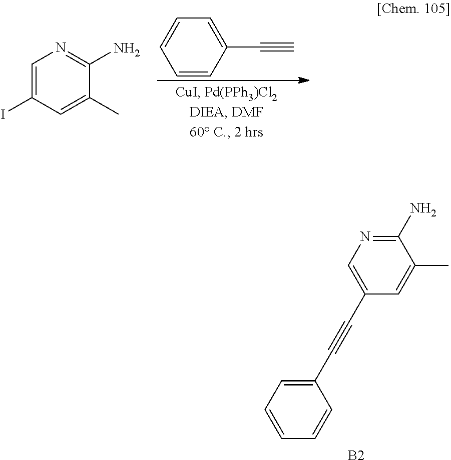

3-methyl-5-(phenylethynyl)pyridin-2-amine (B2): Compound B2 is prepared in a similar manner as Compound B1 to yield the title compound B2 (1807 mg, 68% yield). ES-MS [M+1]+: 209.4, ¹H NMR (400 MHz, CDCl₃) δ 8.15 (s, 1H), 7.51-7.48 (m, 2H), 7.45 (s, 1H), 7.03 (m, 3H), 4.76 (s, 2H), 2.14 (s, 3H).

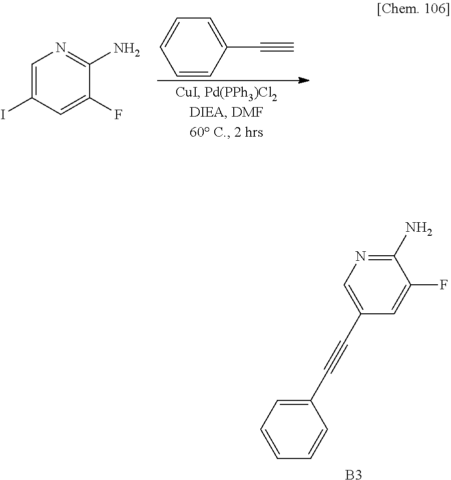

3-fluoro-5-(phenylethynyl)pyridin-2-amine (B3): Compound B3 is prepared in a similar manner as Compound B1 to yield the title compound B3 (26.0 mg, 97% yield). ES-MS [M+1]+: 213.2, ¹H NMR (400 MHz, CDCl₃) δ 8.07 (s, 1H), 7.51-7.49 (m, 2H), 7.36-7.33 (m, 4H), 4.76 (s, 2H).

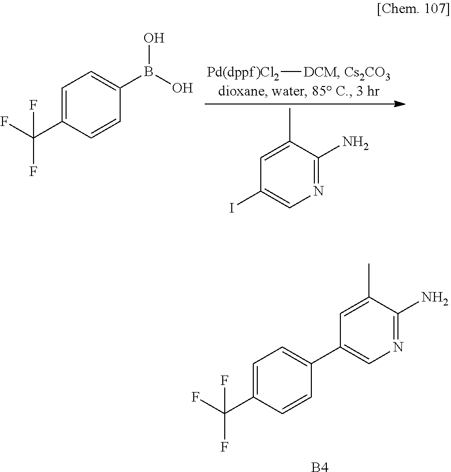

3-methyl-5-(4-(trifluoromethyl)phenyl)pyridin-2-amine (B4): A mixture of 5-iodo-2-amino-3-picoline (100 mg, 0.43 mmol), 4-(trifluoromethyl)phenylboronic acid (162 mg, 0.86 mmol), Pd(dppf)Cl₂-DCM adduct (35 mg, 0.04 mmol), and Cs₂CO₃ (308 mg, 0.94 mmol) in dioxane (4 mL) and water (0.4 mL) was heated to 85° C. for 3 hours. The solution was removed from heat and diluted with water. The dilution was extracted 2×25 mL with EtOAc, and washed with water 2×50 mL. The extraction was concentrated and purified by normal phase flash chromatography (gradient: 0-10% MeOH in DCM). Fractions that contain product were combined and concentrated to yield (Intermediate B4) (108 mg, 99% yield). ES-MS [M+1]+: 253.4.

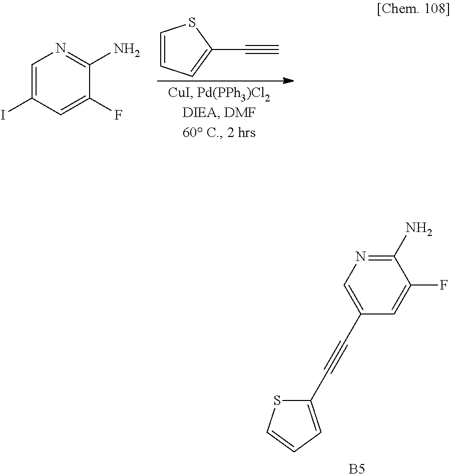

3-fluoro-5-(thiophen-2-ylethynyl)pyridin-2-amine (B5): Compound B5 is prepared in a similar manner as Compound B1 to yield the title compound B5 (1096 mg, 100% yield). ES-MS [M+1]+: 219.2, ¹H NMR (400 MHz, (CD₃)₂SO) S 7.97 (dd, J=1.5, 1.5 Hz, 1H), 7.63 (dd, J=5.1, 1.1 Hz, 1H), 7.54 (dd, J=11.9, 1.8 Hz, 1H), 7.35 (dd, J=3.6, 1.1 Hz, 1H), 7.10 (dd, J=5.1, 3.6 Hz, 1H), 6.76 (s, 2H).

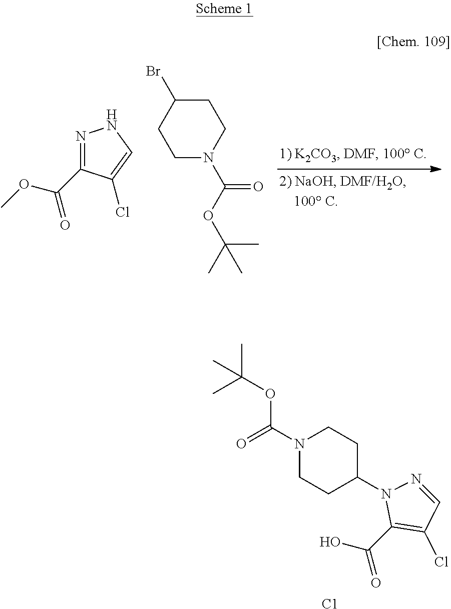

1-(1-(tert-butoxycarbonyl)piperidin-4-yl)-4-chloro-1H-pyrazole-5-carboxylic acid (C1): To a mixture of 1-boc-4-bromopiperidine (2000 mg, 7.57 mmol), methyl 4-chloropyrazole-3-carboxylate (527 mg, 3.3 mmol), and potassium carbonate (885 mg, 6.31 mmol) was added DMF (5 mL) and the mixture was heated to 100° C. After 6 h, sodium hydroxide (667 mg, 16.3 mmol) and water (2.5 mL) were added to the mixture. After an additional 1 h at 100° C., the reaction was removed from the heat source. At rt, the mixture was diluted with water and then 2 mol/L HCl was added dropwise until pH=4-5. After the mixture was extracted with CHCl₃:IPA (3:1, 3×), the collected organic layers were concentrated in vacuo and then purified by reverse phase HPLC (gradient: 25-65% MeCN in water (w/0.1% TFA)). The desired fractions were concentrated to yield C1 as an off-white solid that was carried forward as a TFA salt (904 mg, 36% yield over 2 steps). ES-MS [M+Na]⁺: 352.3, ¹H NMR (400 MHz, CDCl₃) δ 7.54 (s, 1H), 5.26-5.15 (m, 1H), 4.26 (br d, J=12.9 Hz, 2H), 2.97-2.83 (m, 2H), 2.08 (dddd, J=13.1, 12.2, 11.9, 4.2 Hz, 2H), 2.01-1.93 (m, 2H), 1.47 (s, 9H).

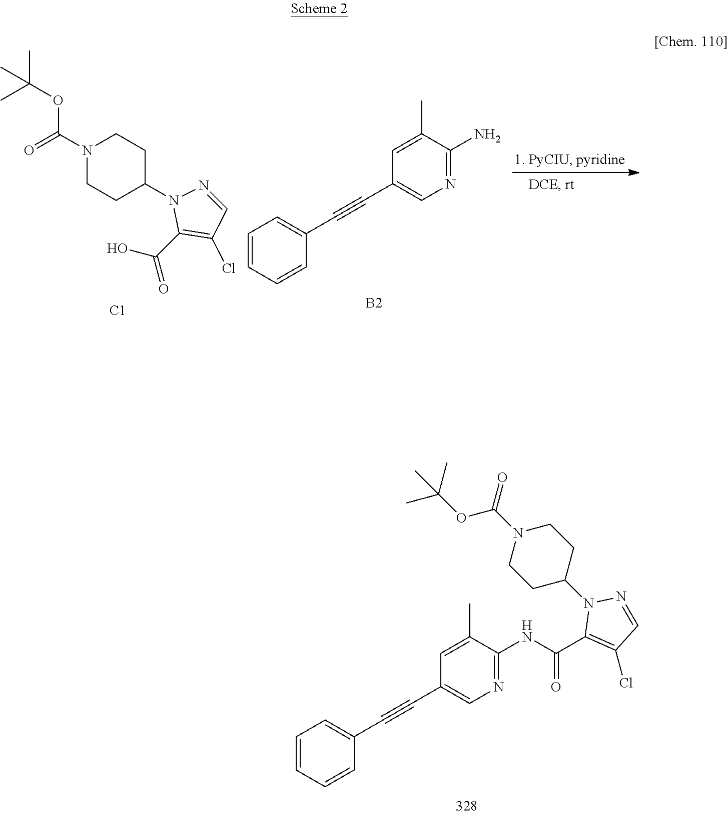

tert-butyl 4-chloro-5-((3-methyl-5-(phenylethynyl)pyridin-2-yl)carbamoyl)-1H-pyrazol-1-yl)piperidine-1-carboxylate (328): To a mixture of C1 (3500 mg, 10.6 mmol), Intermediate B2 (3315 mg, 15.9 mmol), PyClU (7061 mg, 21.2 mmol), and pyridine (8.6 mL, 106.1 mmol) at rt was added DCE (21.3 mL). After 17 h, the reaction mixture was concentrated in vacuo and then purified by reverse phase HPLC (gradient: 60-95% MeCN in water (w/0.1% TFA)) to yield 328 as a tan solid that was carried forward as a TFA salt (2230 mg, 33% yield). ES-MS [M+1]⁺: 520.0, LCMS Retention time: 1.21 min, ¹H NMR (400 MHz, CDCl₃) δ 10.98 (s, 1H), 8.41 (d, J=1.6 Hz, 1H), 7.95 (d, J=1.6 Hz, 1H), 7.68 (s, 1H), 7.60-7.55 (m, 2H), 7.48-7.43 (m, 3H), 4.70-4.59 (m, 1H), 4.04 (d, J=12.0 Hz, 2H), 3.0-2.76 (m, 2H), 2.29 (s, 3H), 2.03-1.80 (m, 4H), 1.41 (s, 9H).

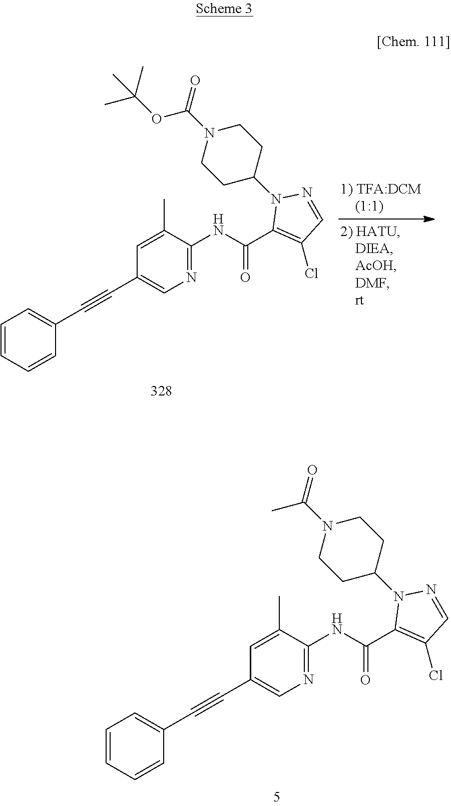 [SMILES: Cc1cc(C#Cc2ccccc2)cnc1NC(=O)c1c(Cl)cnn1C1CCN(C(=O)OC(C)(C)C)CC1 ; CC(=O)N1CCC(n2ncc(Cl)c2C(=O)Nc2ncc(C#Cc3ccccc3)cc2C)CC1]

1-(1-acetylpiperidin-4-yl)-4-chloro-N-(3-methyl-5-(phenylethynyl)pyridin-2-yl)-1H-pyrazole-5-carboxamide (5): To the TFA salt of 328 (4585 mg, 7.23 mmol) was added DCM:TFA (1:1; 10 mL) at rt. After 1 h, the mixture was concentrated in vacuo to give a tan solid. ES-MS [M+1]⁺: 420.4. The resulting crude amine was dissolved in DMF (36 mL) and DIEA (18.9 mL, 108 mmol) was added. To the mixture was added acetic acid (2.1 mL, 36.2 mmol) and HATU (6050 mg, 15.9 mmol). After 30 min, the mixture was concentrated in vacuo and then purified by reverse phase HPLC (gradient: 50-90% MeCN in water (w/0.05% NH₄OH)). The desired fractions were concentrated to give 5 as an off-white solid (2787 mg, 91% over 2 steps). ES-MS [M+1]⁺: 462.0, LCMS Retention time: 1.07 min, ¹H NMR (400 MHz, (CD₃)₂SO) S 10.99 (s, 1H), 8.41 (d, J=1.7 Hz, 1H), 7.95 (dd, J=2.2, 0.7 Hz, 1H), 7.68 (s, 1H), 7.61-7.55 (m, 2H), 7.48-7.43 (m, 3H), 4.77-4.66 (m, 1H), 4.46 (d, J=13.4 Hz, 1H), 3.92 (d, J=13.6 Hz, 1H), 3.24-3.14 (m, 1H), 2.75-2.64 (m, 1H), 2.29 (s, 3H), 2.03 (s, 3H), 2.03-1.94 (m, 3H), 1.82 (dddd, J=12.8, 12.2, 12.1, 4.3 Hz, 1H).

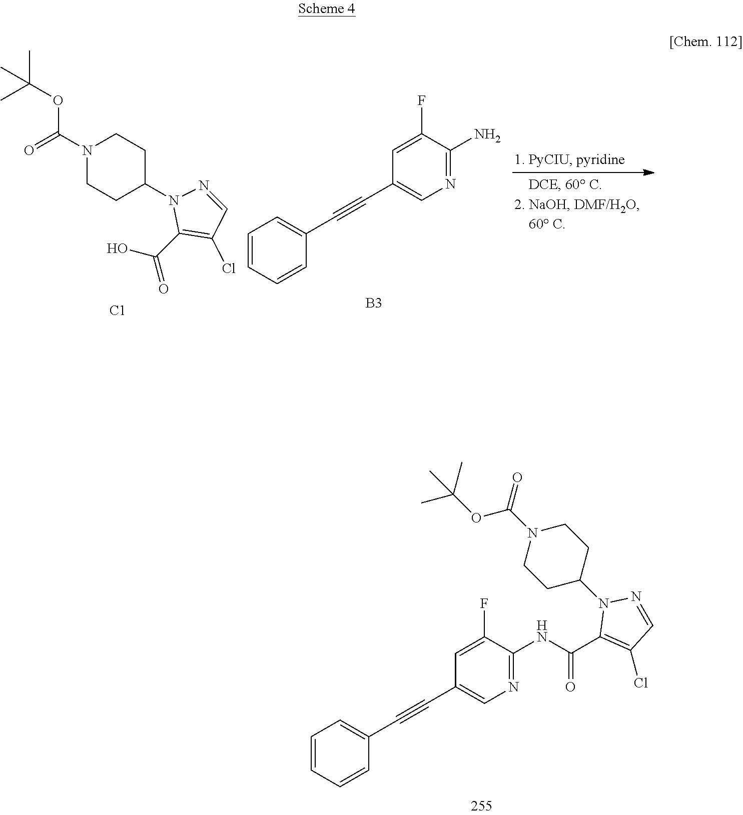

tert-butyl 4-(4-chloro-5-((3-fluoro-5-(phenylethynyl)pyridin-2-yl)carbamoyl)-1H-pyrazol-1-yl)piperidine-1-carboxylate (255): A mixture of C1 (1200 mg, 3.64 mmol), Intermediate B3 (1004 mg, 4.73 mmol), PyClU (2421 mg, 7.28 mmol), and pyridine (3.0 mL, 37.1 mmol) in DCE (12.0 mL) was stirred at 60° C. After 1 h, the mixture was concentrated and to the residue was added DMF (6.0 mL), water (1.0 mL), and NaOH (1000 mg, 24.4 mmol). The mixture was heated to 60° C. for 30 min. After the heat source was removed, the mixture was diluted with water and neutralized with 2 mol/L HCl until pH=4-5. The mixture was extracted with CHCl₃:IPA (3:1; 3×). The collected organic layers were concentrated in vacuo and then purified by reverse phase HPLC (gradient: 60-95% MeCN in water (w/0.1% TFA)) to afford 255 as a tan solid that was carried forward as a TFA salt (2004 mg, 43% yield). ES-MS [M+Na]⁺: 546.4, LCMS Retention time: 1.21 min, ¹H NMR (400 MHz, CDCl₃) δ 10.50 (s, 1H), 8.51 (dd, J=1.7, 0.7 Hz, 1H), 8.28 (s, 1H), 8.08 (dd, J=10.4, 1.8 Hz, 1H), 7.65-7.58 (m, 2H), 7.50-7.45 (m, 3H), 4.49-4.39 (m, 1H), 4.05 (d, J=12.5 Hz, 2H), 3.04-2.82 (br s, 2H), 2.05 (dd, J=12.3, 2.6 Hz, 2H), 1.87 (dddd, 12.9, 12.2, 12.0, 4.1 Hz, 2H), 1.42 (s, 9H).

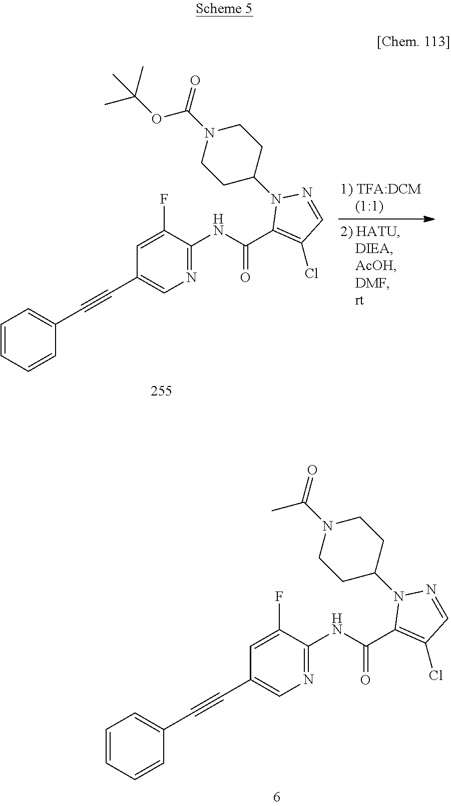 [SMILES: CC(C)(C)OC(=O)N1CCC(n2ncc(Cl)c2C(=O)Nc2ncc(C#Cc3ccccc3)cc2F)CC1 ; CC(=O)N1CCC(n2ncc(Cl)c2C(=O)Nc2ncc(C#Cc3ccccc3)cc2F)CC1]

1-(1-acetylpiperidin-4-yl)-4-chloro-N-(3-fluoro-5-(phenylethynyl)pyridin-2-yl)-1H-pyrazole-5-carboxamide (6): To the TFA salt of 255 (5762 mg, 11.0 mmol) was added DCM:TFA (1:1; 40 mL). After 1 h at rt, the reaction mixture was concentrated in vacuo and washed with diethyl ether to give a tan solid. ES-MS [M+1]+: 424.0. The resulting crude amine (5914.7 mg, 11.0 mmol) was dissolved in DMF (55.0 mL) and DIEA (28.8 mL, 165.3 mmol) was added. The mixture was stirred for 5 min and then acetic acid (3.2 mL, 55.9 mmol) and HATU (9198 mg, 24.2 mmol) were added. The mixture was stirred for 30 min. To the mixture was added NH₄OH (5 mL), and the mixture was stirred for 1 h. The sample was concentrated in vacuo and then purified by reverse phase HPLC (gradient: 50-80% MeCN in water (w/0.05% NH₄OH)). The desired fractions were concentrated to give 6 as a white solid (3300 mg, 82% over 2 steps). ES-MS [M+1]⁺: 466.3, LCMS Retention time: 1.08 min, ¹H NMR (400 MHz, (CD₃)₂SO) δ 11.33 (s, 1H), 8.47 (d, J=1.2 Hz, 1H), 8.12 (dd, J=10.6, 1.7 Hz, 1H), 7.73 (s, 1H), 7.64-7.57 (m, 2H), 7.50-7.45 (m, 3H), 4.78-4.66 (m, 1H), 4.46 (d, J=13.6 Hz, 1H), 3.91 (dd, J=13.7, 2.2 Hz, 1H), 3.22-3.12 (m, 1H), 2.72-2.61 (m, 1H), 2.03 (s, 3H), 2.02-1.92 (m, 3H), 1.80 (dddd, 12.8, 12.7, 12.0, 4.4 Hz, 1H).

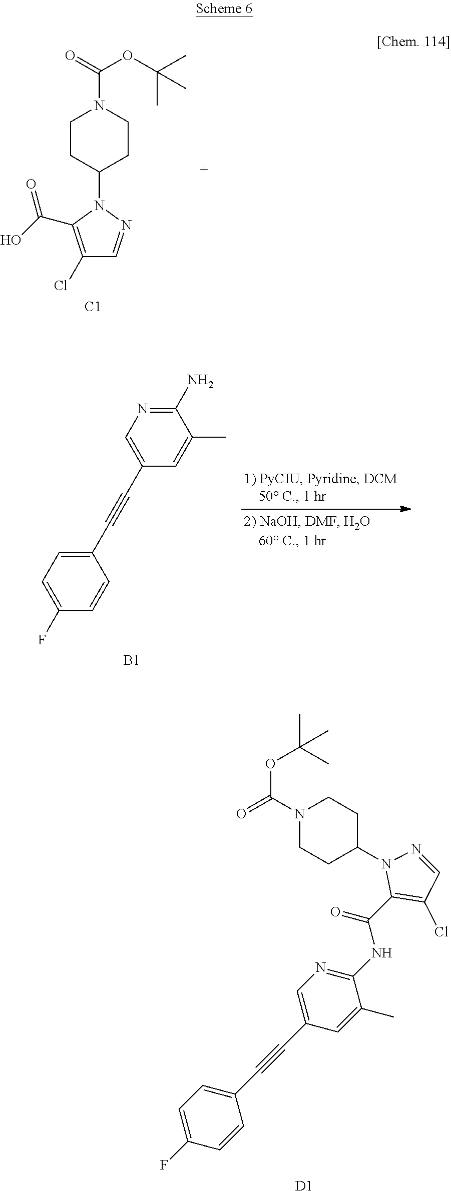

tert-butyl 4-(4-chloro-5-((5-((4-fluorophenyl)ethynyl)-3-methylpyridin-2-yl)carbamoyl)-1H-pyrazol-1-yl)piperidine-1-carboxylate (D1): To a vial was added Intermediate B1 (961 mg, 4.25 mmol), 2-(1-tert-butoxycarbonyl-4-piperidyl)-4-chloro-pyrazole-3-carboxylic acid (C1) (700 mg, 2.12 mmol), pyridine (1.72 mL, 21.2 mmol), PyClU (2118 mg, 6.37 mmol), and DCM (10 mL). The reaction mixture was heated to 50° C. After 1 h, LCMS indicated product formation and an undesired side product. The mixture was concentrated and to the residue was added NaOH (583 mg, 14.2 mmol), DMF (1.5 mL), and H₂O (0.58 mL). The solution was heated to 60° C. After 1 h, LCMS indicated no presence of the undesired side product. The reaction mixture was diluted with H₂O and 1 mol/L HCl was added dropwise until pH=7. After the mixture was extracted with DCM (3×), the collected organic layers were filtered, concentrated and purified by reverse phase HPLC (gradient: 40-100% MeCN in water (0.1% TFA in water)). The desired product was neutralized with a saturated aqueous NaHCO₃ solution and the mixture was extracted with DCM (3×), and the collected organic layers were concentrated to yield (D1) (552 mg, 48% yield). ES-MS [M+1]⁺: 538.2, ¹H NMR (400 MHz, CDCl₃) δ 8.56 (s, 1H), 8.49 (d, J=1.7 Hz, 1H), 7.74 (dd, J=2.0, 0.6 Hz, 1H), 7.56 (s, 1H), 7.54-7.51 (m, 2H), 7.10-7.04 (m, 2H), 5.35 (tt, J=11.2, 4.3 Hz, 1H), 4.24 (br s, 2H), 2.85 (br s, 2H), 2.36 (s, 3H), 2.14-1.99 (m, 4H), 1.46 (s, 9H).

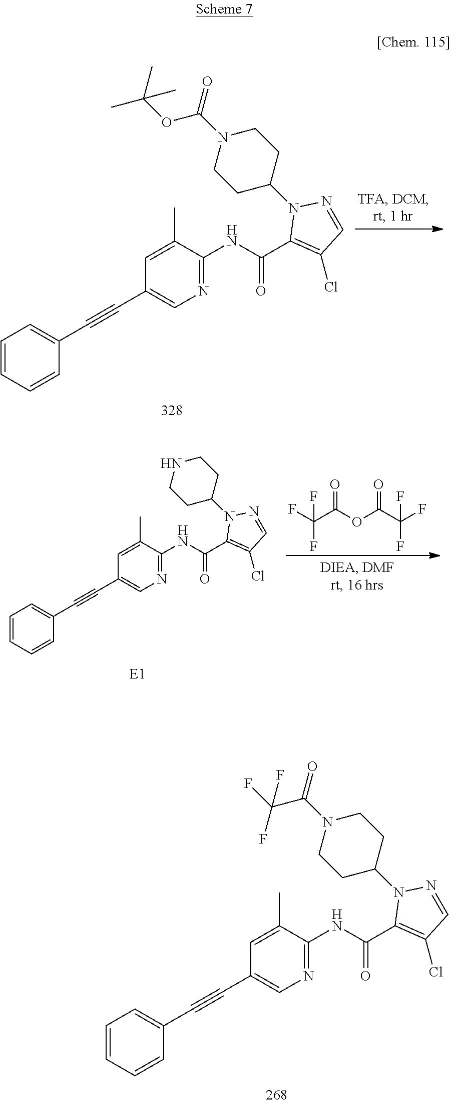

4-chloro-N-[3-methyl-5-(2-phenylethynyl)-2-pyridyl]-2-[1-(2,2,2-trifluoroacetyl)-4-piperidyl]pyrazole-3-carboxamide (El): To a solution of tert-butyl 4-[4-chloro-5-[[3-methyl-5-(2-phenylethynyl)-2-pyridyl]carbamoyl]pyrazol-1-yl]piperidine-1-carboxylate; 2,2,2-trifluoroacetic acid salt (328) (120 mg, 0.19 mmol) in DCM (1.2 mL) was added trifluoroacetic acid (370 mg, 3.26 mmol). After 1 hour at room temperature, the solution was concentrated to yield 4-chloro-N-[3-methyl-5-(2-phenylethynyl)-2-pyridyl]-2-(4-piperidyl)pyrazole-3-carbo xamide; 2,2,2-trifluoroacetic acid salt (E1). To a solution of (E1) (20 mg, 0.04 mmol) and N,N-diisopropylethylamine (24 mg, 0.19 mmol) in THF (0.67 mL) was added trifluoroacetic anhydride (12 mg, 0.06 mmol) at rt. After 16 hours, the solution was purified by reverse phase HPLC (gradient: 30-95% MeCN in water (w/0.05% NH₄OH)) to afford (268) (12 mg, 62% yield). ES-MS [M+1]⁺: 516.3. LCMS Retention time: 1.08 min.

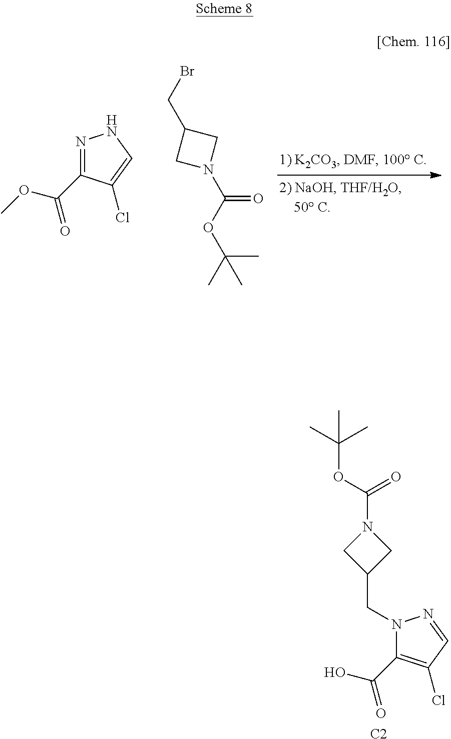

1-((1-(tert-butoxycarbonyl)azetidin-3-yl)methyl)-4-chloro-1H-pyrazole-5-carboxylic acid (C2): A mixture of tert-butyl 3-(bromomethyl)azetidine-1-carboxylate (1402 mg, 5.6 mmol), methyl 4-chloropyrazole-3-carboxylate (750 mg, 4.7 mmol), and potassium carbonate (786 mg, 5.6 mmol) in DMF (10 mL) was heated to 100° C. After 6 h, the reaction mixture was diluted with ethyl acetate, washed with water and brine, then extracted with dichloromethane (3×). The collected organic layers were dried with sodium sulfate, filtered and concentrated in vacuo. The crude product was purified via normal phase column chromatography (gradient: 0-20% EtOAc in Hexanes) to provide the desired ester intermediate, a colorless amorphous solid (912 mg, 59% yield). The intermediate ester was dissolved in THF (2 mL) and 2 mol/L sodium hydroxide (2 mL) was added. The mixture was heated to 50° C. After 3 h, the mixture was diluted with water and the pH of the solution was adjusted to ˜3 via dropwise addition of 2 mol/L HCl. After the reaction mixture was extracted with dichloromethane (3×), the collected organic layers were concentrated in vacuo to afford C2 as a pure, off-white solid (872 mg, 59% yield over 2 steps). ES-MS [M-^{t}Bu]⁺: 260.1, ¹H NMR (400 MHz, CDCl₃) δ 7.50 (s, 1H), 4.75 (m, 2H), 4.03 (dd, J=8.6, 8.6 Hz, 2H), 3.82 (dd, J=9.1, 5.5 Hz, 2H), 3.13 (m, 1H), 1.44 (s, 9H).

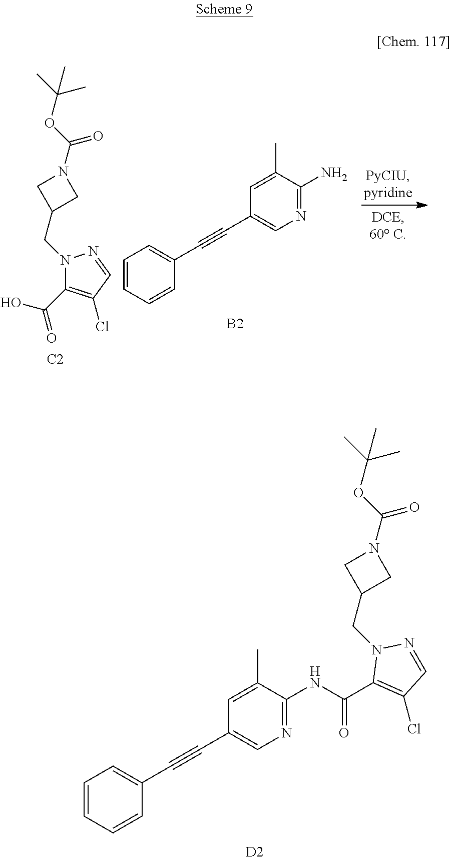

tert-butyl 3-((4-chloro-5-((3-methyl-5-(phenylethynyl)pyridin-2-yl)carbamoyl)-1H-pyrazol-1-yl) methyl)azetidine-1-carboxylate (D2): A mixture of C2 (750 mg, 2.4 mmol), B2 (742 mg, 3.6 mmol), PyClU (1580 mg, 4.8 mmol), and pyridine (1.9 mL, 24.2 mmol) in DCE (21.3 mL) was stirred at 60° C. for 16 h. The sample was concentrated in vacuo and then purified by reverse phase HPLC (gradient: 60-95% MeCN in water (w/0.05% NH₄OH)). The desired fractions were concentrated to yield D2 as a tan solid (456 mg, 38% yield). ES-MS [M+1]⁺: 506.2, ¹H NMR (400 MHz, CDCl₃) δ 8.63 (s, 1H), 8.43 (br. s, 1H), 7.69 (m, 1H), 7.51-7.46 (m, 3H), 7.33-7.28 (m, 3H), 4.74 (d, J=7.4 Hz, 2H), 3.92 (dd, J=8.6, 8.6 Hz, 2H), 3.77-3.71 (dd, J=8.4, 5.3 Hz, 2H), 3.15-3.04 (m, 1H), 2.29 (s, 3H), 1.37 (s, 9H).

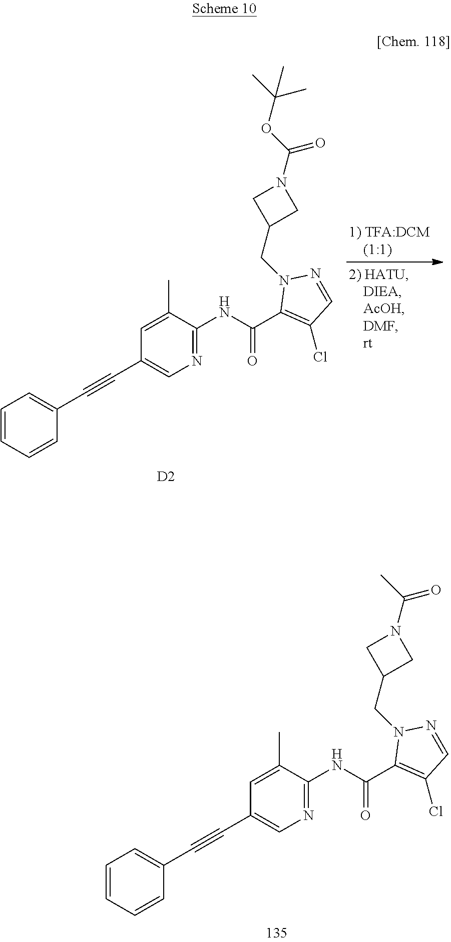 [SMILES: Cc1cc(C#Cc2ccccc2)cnc1NC(=O)c1c(Cl)cnn1CC1CN(C(=O)OC(C)(C)C)C1 ; "D2" ; CC(=O)N1CC(Cn2ncc(Cl)c2C(=O)Nc2ncc(C#Cc3ccccc3)cc2C)C1]

1-((1-acetylazetidin-3-yl)methyl)-4-chloro-N-(3-methyl-5-(phenylethynyl)pyridin-2-yl)-1H-pyrazole-5-carboxamide (135): To D2 (187 mg, 0.37 mmol) was added DCM: TFA (4 mL; 1:1). After 1 h at rt, the mixture was concentrated in vacuo, washed with saturated sodium bicarbonate and extracted with DCM (3×). The collected organic layers were concentrated in vacuo to afford the crude amine. ES-MS [M+1]+: 406.2. The resulting crude amine (150 mg, 0.37 mmol) was dissolved in DMF (3.5 mL) and DIEA (0.14 mL, 0.81 mmol) was added. After 5 min, to the mixture was added acetic acid (2.5 μL, 0.44 mmol) and HATU (168.6 mg, 0.44 mmol). After an additional 30 min, the sample was concentrated in vacuo and then purified by reverse phase HPLC (gradient: 50-90% MeCN in water (w/0.05% NH₄OH)). The desired fractions were concentrated to give 135 as an off-white amorphous solid (100.9 mg, 60% over 2 steps). ES-MS [M+1]⁺: 448.2, LCMS Retention time: 0.97 min, ¹H NMR (400 MHz, (CDCl₃) δ. 8.65 (s, 1H), 8.48 (s, 1H), 7.74 (m, 1H), 7.55-7.51 (m, 3H), 7.37-7.33 (m, 3H), 4.88-4.74 (m, 2H), 4.14 (dd, J=8.1, 8.1 Hz, 1H), 4.09-4.00 (m, 2H), 3.81 (dd, J=10.1, 5.5 Hz, 1H), 3.22 (m, 1H), 2.34 (s, 3H), 1.83 (s, 3H).

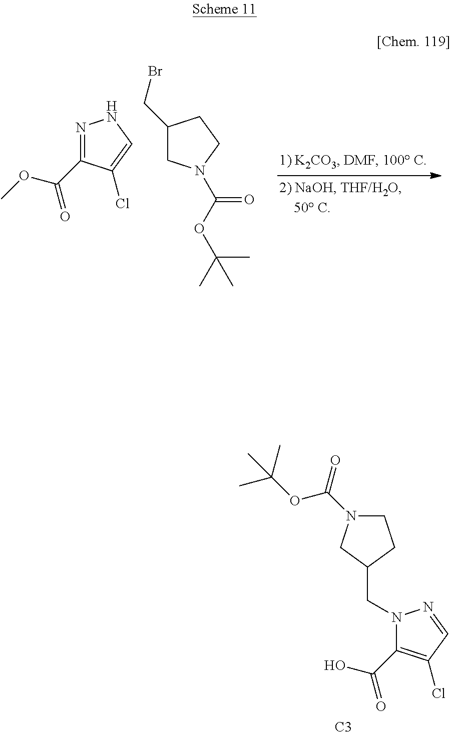

1-((1-(tert-butoxycarbonyl)pyrrolidin-3-yl)methyl)-4-chloro-1H-pyrazole-5-carboxylic acid (C3): A mixture of tert-butyl 3-(bromomethyl)pyrrolidine-1-carboxylate (329 mg, 1.2 mmol), methyl 4-chloropyrazole-3-carboxylate (100 mg, 0.6 mmol), and potassium carbonate (175 mg, 1.2 mmol) in DMF (2 mL) was heated to 100° C. After 6 h, the reaction mixture was diluted with ethyl acetate, washed with water and brine, then extracted with dichloromethane (3×). The collected organic layers were dried with sodium sulfate, filtered and concentrated in vacuo. The crude product was purified via normal phase column chromatography (gradient: 0-20% EtOAc in Hexanes) to provide the methyl ester intermediate, which was obtained as a colorless amorphous solid (84 mg, 39% yield). The intermediate ester was dissolved in THF (2 mL) and 2 mol/L sodium hydroxide (2 mL) was added. After 2 hrs at 50° C., the mixture was diluted with water and 2 mol/L HCl was added dropwise until pH=3. The reaction mixture was extracted with dichloromethane (3×) and the collected organic layers were concentrated in vacuo to afford C3 as a pure, off-white solid (80 mg, 39% yield over 2 steps). ES-MS [M-^{t}Bu]⁺: 274.0, ¹H NMR (400 MHz, CDCl₃) δ 7.88 (br. s, 1H), 7.44 (m, 1H), 4.59 (m, 1H), 4.44 (m, 1H), 3.52-3.32 (m, 2H), 3.25 (m, 1H), 3.08 (m, 1H), 2.70 (m, 1H), 1.83 (m, 1H), 1.62 (m, 1H), 1.39 (s, 9H).

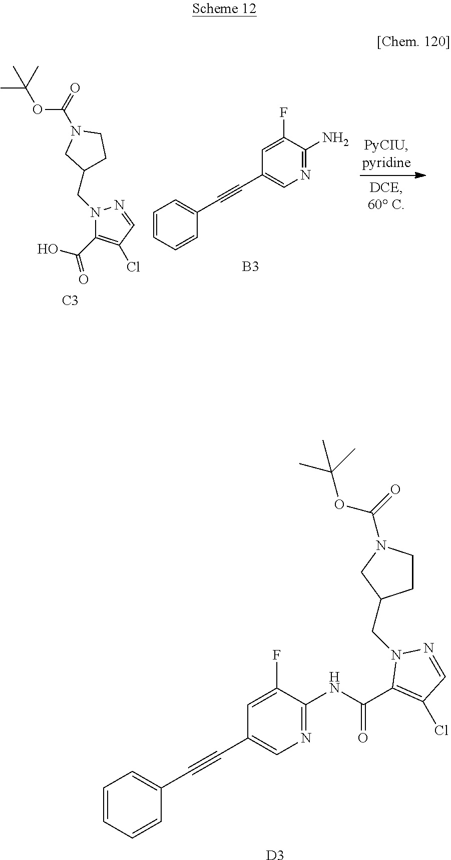

tert-butyl 3-((4-chloro-5-((3-fluoro-5-(phenylethynyl)pyridin-2-yl)carbamoyl)-1H-pyrazol-1-yl) methyl)pyrrolidine-1-carboxylate (D3): A mixture of Intermediate B3 (25 mg, 0.08 mmol), C3 (57 mg, 0.27 mmol), PyClU (50 mg, 0.15 mmol), and pyridine (0.06 mL, 0.15 mmol) in DCE (1.5 mL) was stirred at 60° C. for 2 h. The sample was concentrated in vacuo and then purified by reverse phase HPLC (gradient: 40-95% MeCN in water (w/0.05% NH₄OH)). The desired fractions were concentrated to yield D3 as a pale yellow oil (28 mg, 71% yield). ES-MS [M+1]⁺: 524.0, ¹H NMR (400 MHz, CDCl₃) δ 8.88 (br. s, 1H), 8.41 (d, J=1.6 Hz, 1H), 7.58 (dd, J=10.3, 1.5 Hz, 2H), 7.52-7.47 (m, 3H), 7.36-7.30 (m, 3H), 4.69-4.54 (m, 2H), 3.50-3.36 (m, 2H), 3.24 (m, 1H), 3.09 (m, 1H), 1.86 (m, 1H), 1.63 (m, 1H), 1.39 (s, 9H).

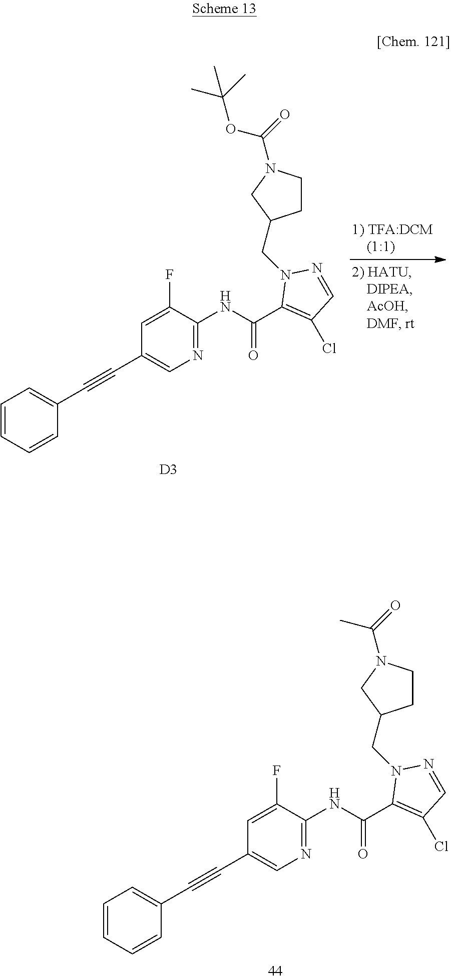 [SMILES: CC(C)(C)OC(=O)N1CCC(Cn2ncc(Cl)c2C(=O)Nc2ncc(C#Cc3ccccc3)cc2F)C1 ; "D3" ; CC(=O)N1CCC(Cn2ncc(Cl)c2C(=O)Nc2ncc(C#Cc3ccccc3)cc2F)C1 ; "44"]

1-((1-acetylpyrrolidin-3-yl)methyl)-4-chloro-N-(3-fluoro-5-(phenylethynyl)pyridin-2-yl)-1H-pyrazole-5-carboxamide (44): To D3 (11 mg, 0.03 mmol) was added DCM: TFA (1:1; 2 mL). After 1 h at rt, the reaction mixture was concentrated in vacuo, washed with saturated aqueous sodium bicarbonate and extracted with DCM (3×). The combined organic layers were concentrated in vacuo to afford the crude amine. ES-MS [M+1]+: 424.0. The resulting crude amine (10 mg, 0.02 mmol) was dissolved in DMF (1.0 mL) and DIEA (8.8 μL, 0.05 mmol) was added. After 5 min, to the mixture was added acetic acid (1.5 μL, 0.03 mmol) and HATU (10.5 mg, 0.03 mmol). After an additional 30 min, the mixture was concentrated in vacuo and then purified by reverse phase HPLC (gradient: 35-90% MeCN in water (w/0.05% NH₄OH)). The desired fractions were concentrated to give 44 as an off-white amorphous solid (7.6 mg, 65% over 2 steps). ES-MS [M+1]⁺: 466.2, LCMS Retention time: 1.18 min, ¹H NMR (400 MHz, (CDCl₃) S 8.89 (br. s, 1H), 8.42 (s, 1H), 7.60 (ddd, J=10.2, 3.3, 1.8 Hz, 1H), 7.54-7.49 (m, 3H), 7.37-7.32 (m, 3H), 4.70 (ddd, J=13.4, 9.8, 6.5 Hz, 1H), 4.57 (dt, J=13.4, 7.5 Hz, 1H), 3.66-3.49 (m, 2H), 3.48-3.30 (m, 2H), 3.29-3.18 (m, 1H), 2.93-2.80 (m, 1H), 1.98 (m, 3H), 1.83-1.70 (m, 1H).

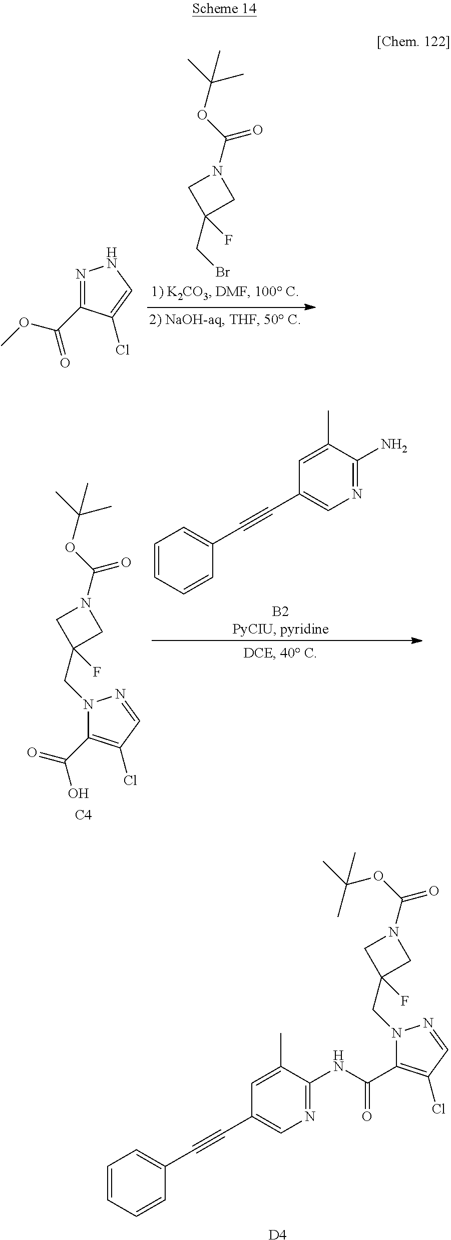

tert-butyl 3-((4-chloro-5-((3-methyl-5-(phenylethynyl)pyridin-2-yl)carbamoyl)-1H-pyrazol-1-yl) methyl)-3-fluoroazetidine-1-carboxylate (D4): A mixture of tert-butyl 3-(bromomethyl)azetidine-1-carboxylate (1402 mg, 5.6 mmol), methyl 4-chloropyrazole-3-carboxylate (750 mg, 4.7 mmol), and potassium carbonate (786 mg, 5.6 mmol) in DMF (10 mL) was stirred at 100° C. for 6 h. The reaction mixture was diluted with ethyl acetate, washed with water and brine, then extracted with dichloromethane (2×). The organic layers were dried with sodium sulfate, filtered, then removed in vacuo. Crude product was purified via silica gel chromatography using 100:0 to 80:20 hexanes:ethyl acetate to elute the pure ester intermediate, which was obtained as a colorless amorphous solid (912 mg). The intermediate ester was dissolved in THF (2 mL) and 2 mol/L sodium hydroxide (2 mL), then stirred for 2 hours at 50° C. Upon completion of the reaction, the solvent was partially removed in vacuo and the mixture was diluted with water. The pH of the solution was adjusted to ˜3 via slow addition of 2 mol/L HCl, then product was extracted with dichloromethane. The organics were concentrated in vacuo to afford C4 (872 mg, 59% yield over 2 steps). ES-MS [M-^{t}Bu]⁺: 278.2, ¹H NMR (400 MHz, d6-DMSO) δ 7.81 (s, 1H), 5.07 (d, J=20.5 Hz, 2H), 4.20 (dd, J=19.7, 10.7 Hz, 2H), 3.89 (dd, J=21.5, 10.4 Hz, 2H), 1.39 (s, 9H).

Compound D4 is prepared in a similar manner as Compound 328 to yield the title compound (D4) (128 mg, 41% yield). ES-MS [M+1]⁺: 524.4.

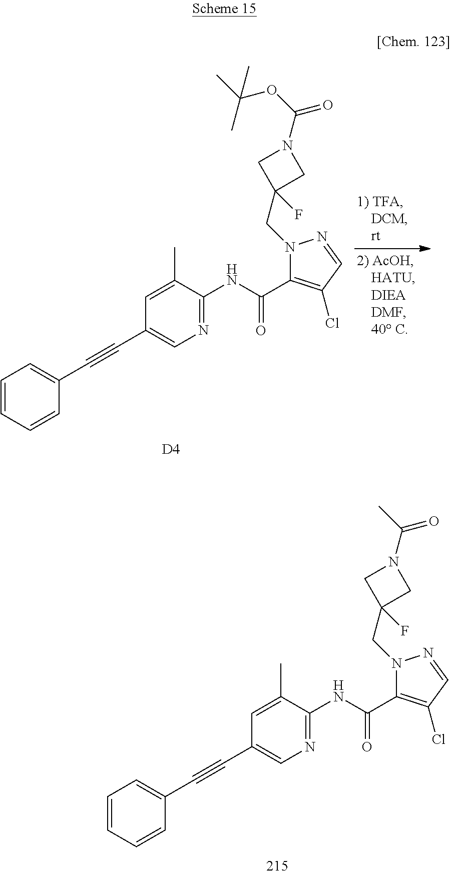 [SMILES: Cc1cc(C#Cc2ccccc2)cnc1NC(=O)c1c(Cl)cnn1CC1(F)CN(C(=O)OC(C)(C)C)C1 ; "D4" ; CC(=O)N1CC(F)(Cn2ncc(Cl)c2C(=O)Nc2ncc(C#Cc3ccccc3)cc2C)C1 ; "215"]

1-((1-acetyl-3-fluoroazetidin-3-yl)methyl)-4-chloro-N-(3-methyl-5-(phenylethynyl)pyridin-2-yl)-1H-pyrazole-5-carboxamide (215): Compound 215 is prepared in a similar manner as Compound 5 to yield the title compound (215) (68 mg, 60% yield). ES-MS [M+H]⁺: 466.3, LCMS Retention time: 1.14 min, ¹H NMR (400 MHz, CDCl₃) δ 8.56 (s, 1H), 8.49 (s, 1H), 7.76 (d, J=1.4 Hz, 1H), 7.61 (s, 1H), 7.56-7.54 (m, 2H), 7.38-7.37 (m, 3H), 5.25 (dd, J=14.9, 14.9 Hz, 1H), 5.06 (dd, J=21.3, 14.6 Hz, 1H), 4.48 (dd, J=17.7, 10.1 Hz, 1H), 4.27-4.17 (m, 2H), 4.12 (dd, J=21.4, 11.5 Hz, 1H), 2.35 (s, 3H), 1.90 (s, 3H).

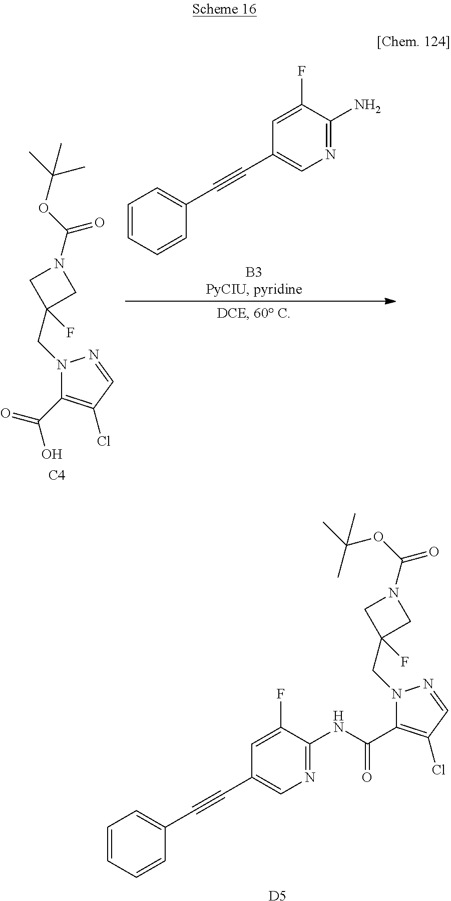

tert-butyl 3-((4-chloro-5-((3-fluoro-5-(phenylethynyl)pyridin-2-yl)carbamoyl)-1H-pyrazol-1-yl) methyl)-3-fluoroazetidine-1-carboxylate (D5): To a mixture of PyClU (223 mg, 0.67 mmol), 3-fluoro-5-(2-phenylethynyl)pyridin-2-Amine B3 (107 mg, 0.50 mmol) and 2-[(1-tert-butoxycarbonyl-3-fluoro-azetidin-3-yl)methyl]-4-chloro-pyrazole-3-carboxylic acid C4 (150 mg, 0.34 mmol) in DCE (2.5 mL) was added pyridine (0.30 mL, 3.43 mmol). After 17 h at 60° C., the mixture was cooled to rt and NH₄OH (0.5 mL, 12.8 mmol) was added to the solution. After 1 h at rt, the solution was concentrated under reduced pressure. The resulting residue was dissolved in DMSO and the desired product was purified by reverse phase HPLC (gradient: 60-95% MeCN in water (w/0.1% TFA)). The fractions with desired product were combined and concentrated to yield the title compound D5 as a tan solid that was carried forward as a TFA salt (113 mg, 53% yield). ES-MS [M+H]⁺: 528.3, ¹H NMR (400 MHz, ((CD₃)₂SO) S 11.3 (br s, 1H), 8.48 (d, J=1.5 Hz, 1H), 8.11 (dd, J=10.5, 1.8 Hz, 1H), 7.82 (s, 1H), 7.64-7.58 (m, 2H), 7.50-7.43 (m, 3H), 4.88 (d, J=21.5 Hz, 2H), 4.20 (dd, J=19.4, 10.3 Hz, 2H), 3.88 (dd, J=21.2, 10.4 Hz, 2H), 1.38 (s, 9H).

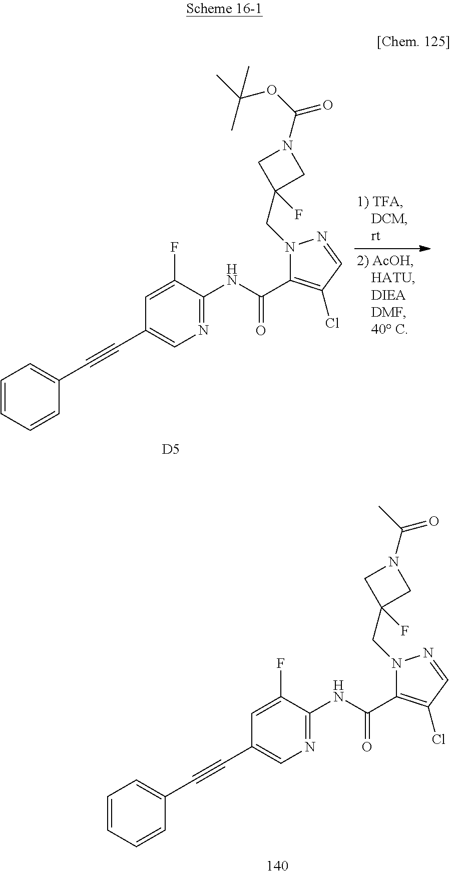 [SMILES: CC(C)(C)OC(=O)N1CC(F)(Cn2ncc(Cl)c2C(=O)Nc2ncc(C#Cc3ccccc3)cc2F)C1 ; "D5" ; CC(=O)N1CC(F)(Cn2ncc(Cl)c2C(=O)Nc2ncc(C#Cc3ccccc3)cc2F)C1 ; "140"]

1-((1-acetyl-3-fluoroazetidin-3-yl)methyl)-4-chloro-N-(3-fluoro-5-(phenylethynyl)pyridin-2-yl)-1H-pyrazole-5-carboxamide (140): To a solution of tert-butyl 3-((4-chloro-5-((3-fluoro-5-(phenylethynyl)pyridin-2-yl)carbamoyl)-1H-pyrazol-1-yl) methyl)-3-fluoroazetidine-1-carboxylate D5; 2,2,2-trifluoroacetic acid salt (83 mg, 0.13 mmol) in DCM (1 mL) was added trifluoroacetic acid (1 mL). After 1 h at rt, the solution was concentrated under reduced pressure. The resulting residue was diluted in DMF (1 mL). To the dilution was added DIEA (0.414 mL, 2.38 mmol), HATU (108 mg, 0.13 mmol), and acetic acid (0.036 mL, 0.63 mmol). After 2 h at rt, the solution was filtered and purified by reverse phase HPLC (gradient: 20-70% MeCN in water (w/0.05% NH₄OH)). The fractions with desired product were concentrated to yield the title compound 140 as an off-white solid (52 mg, 93% yield). ES-MS [M+1]⁺: 470.3, LCMS Retention time: 1.02 min, ¹H NMR (400 MHz, (CD₃)₂SO) S 11.32 (br s, 1H), 8.48 (d, J=1.0 Hz, 1H), 8.10 (br d, J=10.3 Hz, 1H), 7.81 (s, 1H), 7.64-7.58 (m, 2H), 7.51-7.44 (m, 3H), 4.90 (d, J=21.6 Hz, 2H), 4.46 (dd, J=19.3, 10.6 Hz, 1H), 4.26-4.12 (m, 2H), 3.85 (dd, J=21.6, 11.1 Hz, 1H), 1.79 (s, 3H).

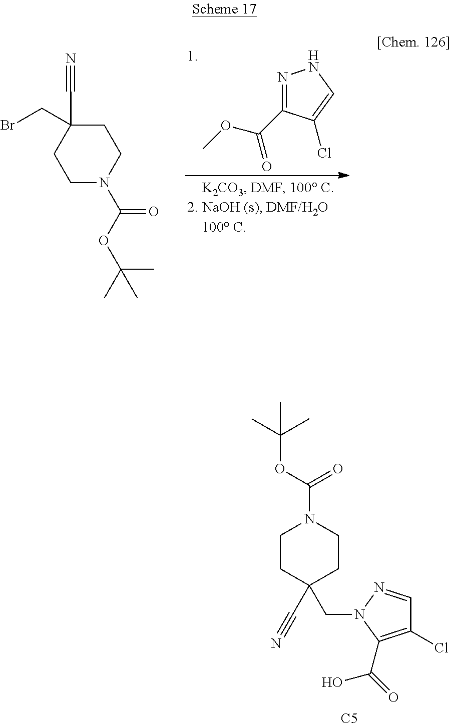

1-((1-(tert-butoxycarbonyl)-4-cyanopiperidin-4-yl)methyl)-4-chloro-1H-pyrazole-5-carboxylic acid (C5): Compound C5 is prepared in a similar manner as Compound C1 to yield the title compound (C5) as an off-white solid that was carried forward as a TFA salt (352 mg, 16% yield over 2 steps). ES-MS [M-tBu+2H]⁺: 313.4, ¹H NMR (400 MHz, ((CD₃)₂SO)) S 7.85 (s, 1H), 4.83 (s, 2H), 3.99 (d, J=13.3 Hz, 2H), 2.82 (br s, 2H), 1.78 (d, J=13.3 Hz, 2H), 1.60 (ddd, J=14.1, 13.2, 4.0 Hz, 2H), 1.40 (s, 9H).

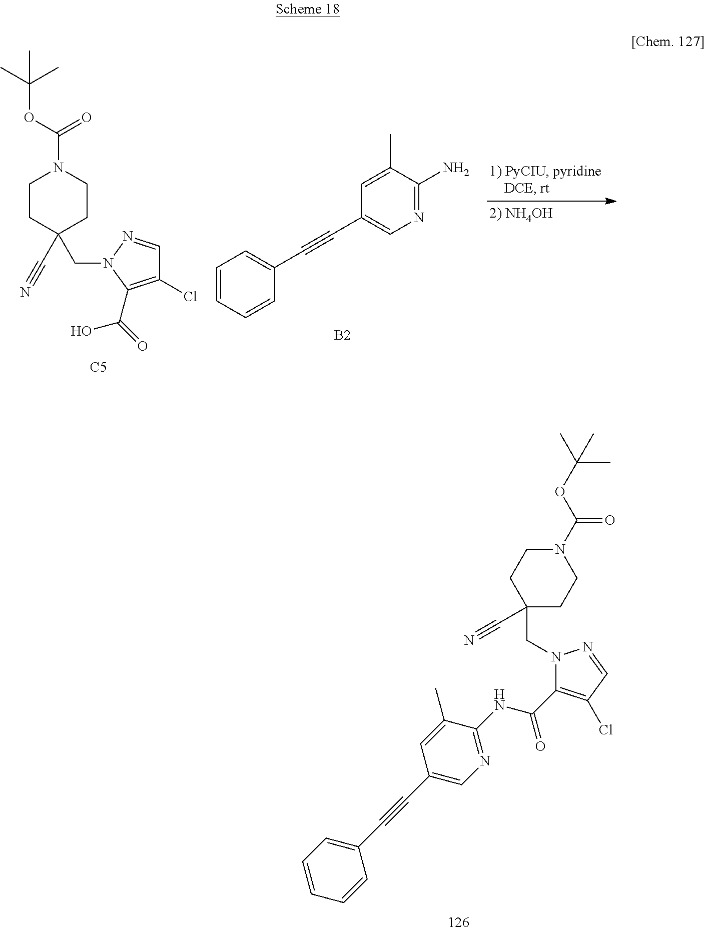

tert-butyl 4-((4-chloro-5-((3-methyl-5-(phenylethynyl)pyridin-2-yl)carbamoyl)-1H-pyrazol-1-yl) methyl)-4-cyanopiperidine-1-carboxylate (126): Compound 126 was prepared using a similar procedure described for 328. Compound 126 was obtained as a tan solid that was carried forward as a TFA salt (185 mg, 64% yield). ES-MS [M+1]⁺: 559.3, LCMS Retention time: 1.27 min, ¹H NMR (400 MHz, ((CD₃)₂SO)) S 10.92 (s, 1H), 8.42 (d, J=1.7 Hz, 1H), 7.96 (dd, J=2.1, 0.6 Hz, 1H), 7.83 (s, 1H), 7.62-7.55 (m, 2H), 7.61-7.55 (m, 2H), 7.48-7.43 (m, 3H), 4.64 (s, 2H), 4.00 (d, J=12.6 Hz, 2H), 2.86 (br s, 2H), 2.28 (s, 3H), 1.86 (ad, J=13.1 Hz, 2H), 1.59 (ddd, J=14.3, 13.4, 4.1 Hz, 2H), 1.39 (s, 9H).

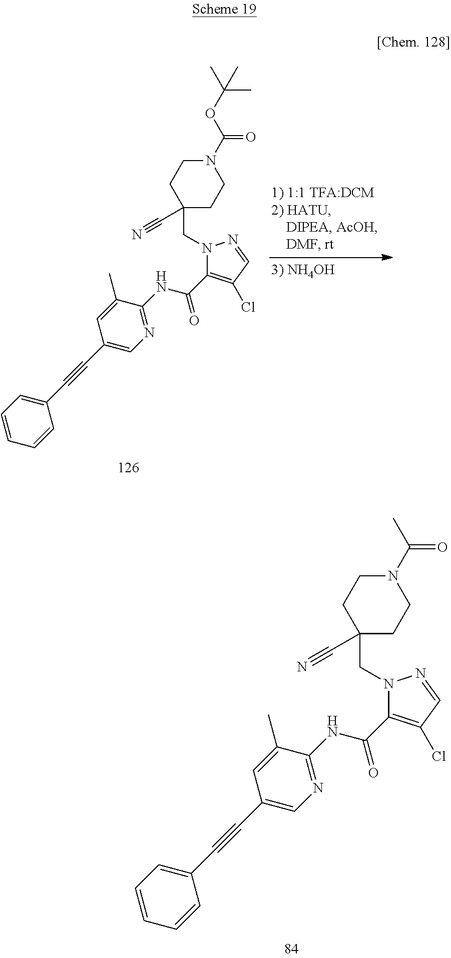 [SMILES: Cc1cc(C#Cc2ccccc2)cnc1NC(=O)c1c(Cl)cnn1CC1(C#N)CCN(C(=O)OC(C)(C)C)CC1 ; CC(=O)N1CCC(C#N)(Cn2ncc(Cl)c2C(=O)Nc2ncc(C#Cc3ccccc3)cc2C)CC1]

1-((1-acetyl-4-cyanopiperidin-4-yl)methyl)-4-chloro-N-(3-methyl-5-(phenylethynyl)pyridin-2-yl)-1H-pyrazole-5-carboxamide (84): Compound 84 is prepared in a similar manner as Compound 5 to yield the title compound (129 mg, 98% yield over 2 steps). ES-MS [M+1]⁺: 501.4, LCMS Retention time: 1.04 min, ¹H NMR (400 MHz, (CD₃)₂SO) S 10.90 (s, 1H), 8.43 (d, J=1.4 Hz, 1H), 7.95 (dd, J=2.1, 0.6, Hz, 1H), 7.83 (s, 1H), 7.63-7.55 (m, 2H), 7.48-7.43 (m, 3H), 4.63 (s, 2H), 4.42 (br d, J=13.7 Hz, 1H), 3.90 (br d, J=14.2 Hz, 1H), 3.21-3.10 (m, 1H), 2.70-2.60 (m, 1H), 2.28 (s, 3H), 2.00 (s, 3H), 1.93 (br d, J=13.5 Hz, 1H), 1.86 (br d, J=14.2 Hz, 1H), 1.71 (ddd, J=14.1, 13.2, 4.0 Hz, 1H), 1.53 (ddd, J=14.1, 13.2, 4.0 Hz, 1H).

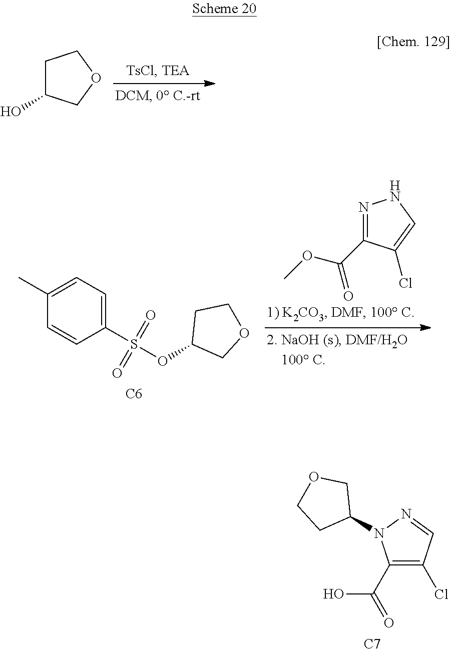

(R)-tetrahydrofuran-3-yl 4-methylbenzenesulfonate (C6): Compounds C6 was prepared using a similar procedure described for C12. Compounds C6 was obtained as a clear, colorless oil (198 mg, 72% yield). ES-MS [M+Na]⁺: 265.4, ¹H NMR (400 MHz, ((CD₃)₂SO)) S 7.81 (d, J=8.2 Hz, 2H), 7.49 (d, J=8.2 Hz, 2H), 5.12-5.09 (m, 1H), 3.79-3.63 (m, 4H), 2.43 (s, 3H), 2.13-2.02 (m, 1H), 1.93-1.84 (m, 1H).

(S)-4-chloro-1-(tetrahydrofuran-3-yl)-1H-pyrazole-5-carboxylic acid (C7): Compounds C7 was prepared using a similar procedure described for C13. Compound C7 was obtained as an off-white solid that was carried forward as a TFA salt (188 mg, 12% yield over 2 steps). ES-MS [M+H]⁺: 217.3, ¹H NMR (400 MHz, ((CD₃)₂SO)) δ 7.74 (s, 1H), 5.76-5.68 (m, 1H), 3.99 (dd, J=9.5, 6.3 Hz, 1H), 3.95 (dd, J=15.3, 7.6 Hz, 1H), 3.85-3.77 (m, 2H), 2.42-2.25 (m, 2H).

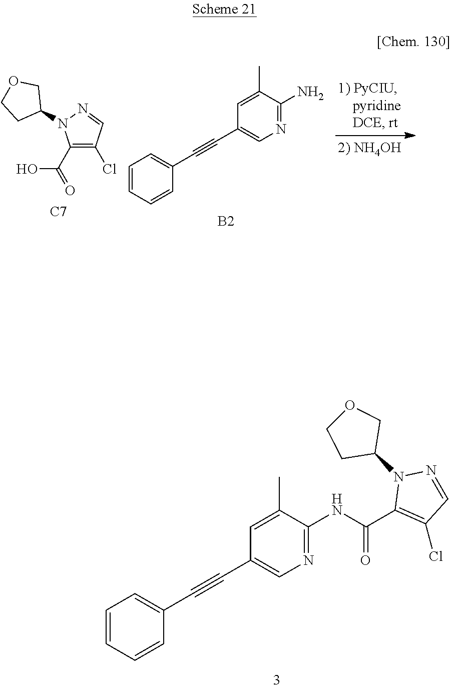

(S)-4-chloro-N-(3-methyl-5-(phenylethynyl)pyridin-2-yl)-1-(tetrahydrofuran-3-yl)-1H-pyrazole-5-carboxamide (3): Compound 3 was prepared using a similar procedure described for C13. Compound 3 was obtained as an off-white solid (17 mg, 62% yield). ES-MS [M+1]⁺: 407.4, LCMS Retention time: 1.09 min, ¹H NMR (400 MHz, CDCl₃) δ 8.50 (s, 1H), 7.87 (s, 1H), 7.58 (s, 1H), 7.57-7.53 (m, 2H), 7.61-7.55 (m, 2H), 7.41-7.36 (m, 3H), 5.93-5.85 (m, 1H), 4.23-4.05 (m, 3H), 3.95 (td, J=8.2, 5.1 Hz, 1H), 2.59-2.50 (m, 1H), 2.46-2.40 (m, 1H), 1.38 (s, 3H).

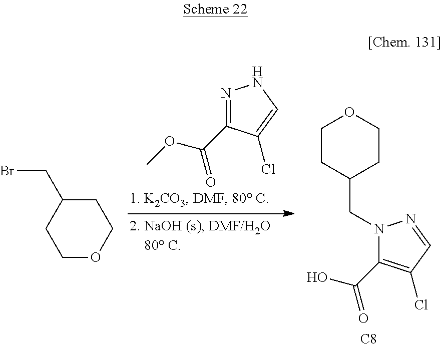

4-chloro-1-((tetrahydro-2H-pyran-4-yl)methyl)-1H-pyrazole-5-carboxylic acid (C8): Compounds C8 was prepared using a similar procedure described for C1 but at 80° C. Compound C8 was obtained as a clear, colorless amorphous solid that was carried forward as a TFA salt (151 mg, 34% yield over 2 steps). ES-MS [M-tBu+2H]⁺: 245.2, ¹H NMR (400 MHz, ((CD₃)₂SO)) δ 7.72 (s, 1H), 4.38 (d, J=7.2 Hz, 2H), 3.80 (ddd, J=11.4, 4.1, 1.7 Hz, 2H), 3.22 (td, J=11.6, 2.2 Hz, 2H), 2.10-1.97 (m, 1H), 1.37-1.29 (m, 2H), 1.22 (qd, 11.6, 4.5 Hz, 2H).

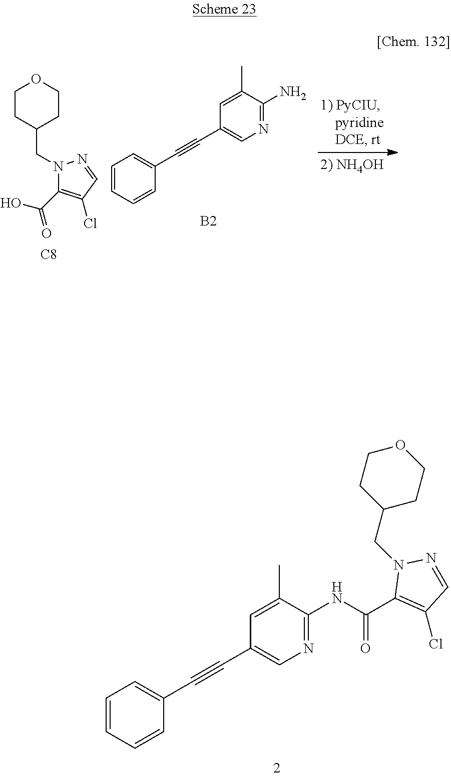

4-chloro-N-(3-methyl-5-(phenylethynyl)pyridin-2-yl)-1-((tetrahydro-2H-pyran-4-yl)methyl)-1H-pyrazole-5-carboxamide (2): Compound 2 was prepared using a similar procedure described for 328. Compound 2 was obtained as a beige solid (5 mg, 20% yield). ES-MS [M+1]⁺: 435.4, LCMS Retention time: 1.13 min, ¹H NMR (400 MHz, CDCl₃) δ 8.66 (br s, 1H), 8.50 (s, 1H), 7.77 (d, J=1.1 Hz, 1H), 7.58-7.51 (m, 3H), 7.41-7.35 (m, 3H), 4.52 (d, J=7.2 Hz, 2H), 3.98-3.90 (m, 2H), 3.33 (ddd, J=11.8, 11.4, 2.9 Hz, 2H), 2.36 (s, 3H), 2.30-2.16 (m, 1H), 1.51-1.37 (m, 4H).

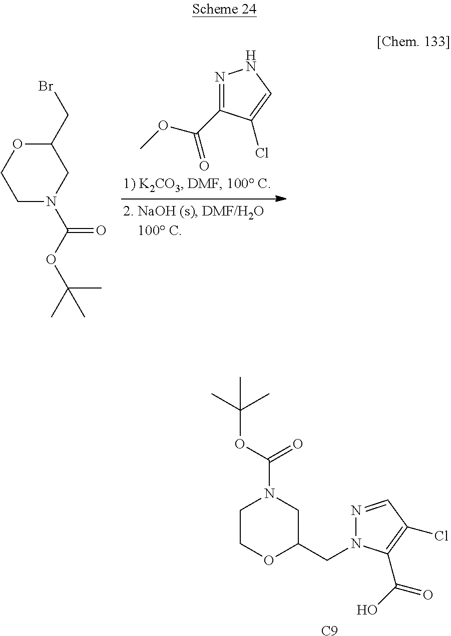

1-((4-(tert-butoxycarbonyl)morpholin-2-yl)methyl)-4-chloro-1H-pyrazole-5-carboxylic acid (C9): Compound C9 was prepared using a similar procedure described for C1. Compound C9 was obtained as a white solid that was carried forward as a TFA salt (640 mg, 39% yield over 2 steps). ES-MS [M+Na]⁺: 369.3, ¹H NMR (400 MHz, ((CD₃)₂SO)) δ 7.68 (s, 1H), 4.62 (dd, J=13.8, 7.0 Hz, 1H), 4.51 (dd, J=13.8, 5.0 Hz, 1H), 3.80-3.60 (m, 4H), 3.30 (dd, J=11.5, 2.8 Hz, 3H), 1.38 (s, 9H).

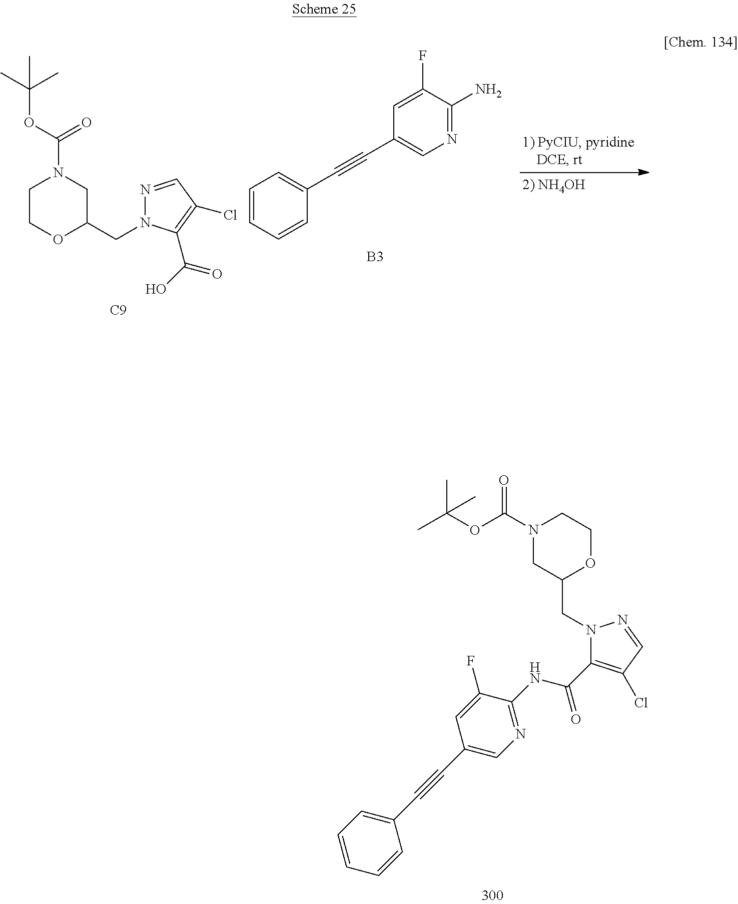

tert-butyl 2-((4-chloro-5-((3-fluoro-5-(phenylethynyl)pyridin-2-yl)carbamoyl)-1H-pyrazol-1-yl) methyl)morpholine-4-carboxylate (300): Compound 300 is prepared in a similar manner as Compound 328 to yield the title compound 300 (73 mg, 36% yield). ES-MS [M+1]⁺: 540.4, LCMS Retention time: 1.12 min, ¹H NMR (400 MHz, (CD₃)₂SO) δ ¹H NMR (400 MHz, DMSO) δ 11.21 (s, 1H), 8.46 (s, 1H), 8.08 (d, J=10.5 Hz, 1H), 7.73 (s, 1H), 7.66-7.55 (m, 2H), 7.51-7.42 (m, 3H), 4.42 (d, J=5.9 Hz, 2H), 3.85-3.73 (m, 2H), 3.72-3.61 (m, 2H), 2.94-2.76 (m, 1H), 2.69-2.41 (m, 2H), 1.38 (s, 9H).

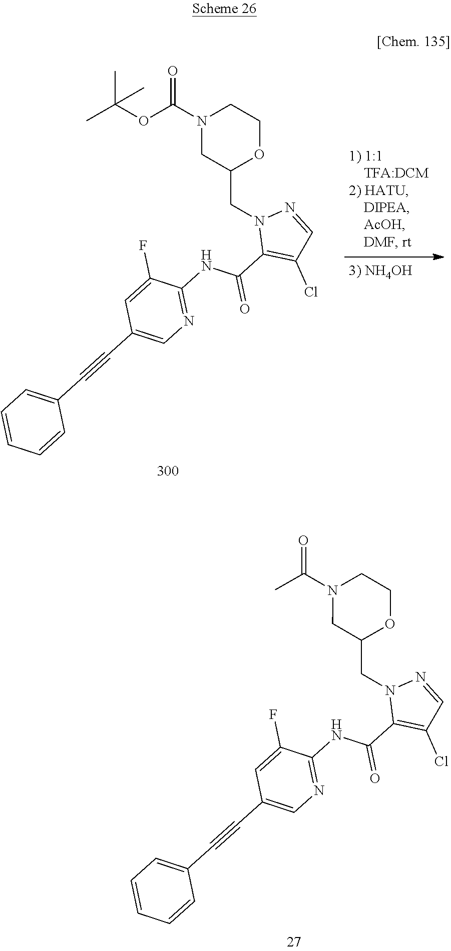 [SMILES: CC(C)(C)OC(=O)N1CCOC(Cn2ncc(Cl)c2C(=O)Nc2ncc(C#Cc3ccccc3)cc2F)C1 ; CC(=O)N1CCOC(Cn2ncc(Cl)c2C(=O)Nc2ncc(C#Cc3ccccc3)cc2F)C1]

1-((4-acetylmorpholin-2-yl)methyl)-4-chloro-N-(3-fluoro-5-(phenylethynyl)pyridin-2-yl)-1H-pyrazole-5-carboxamide (27): Compound 27 is prepared in a similar manner as Compound 5 to yield the title compound 27 (13 mg, 61% yield). ES-MS [M+1]⁺: 482.2, LCMS Retention time: 1.02 min, ¹H NMR (400 MHz, (CD₃)₂SO) δ 11.21 (s, 1H), 8.49 (d, J=5.2 Hz, 1H), 8.10 (d, J=10.5 Hz, 1H), 7.75 (s, 1H), 7.65-7.58 (m, 2H), 7.52-7.44 (m, 3H), 4.50-4.39 (m, 2H), 4.15 (dd, J=58.6, 13.2 Hz, 1H), 3.87-3.60 (m, 3H), 3.44-3.22 (m, 1H), 3.18-2.83 (m, 1H), 2.73-36 (m, 1H), 1.99 (s, 3H).

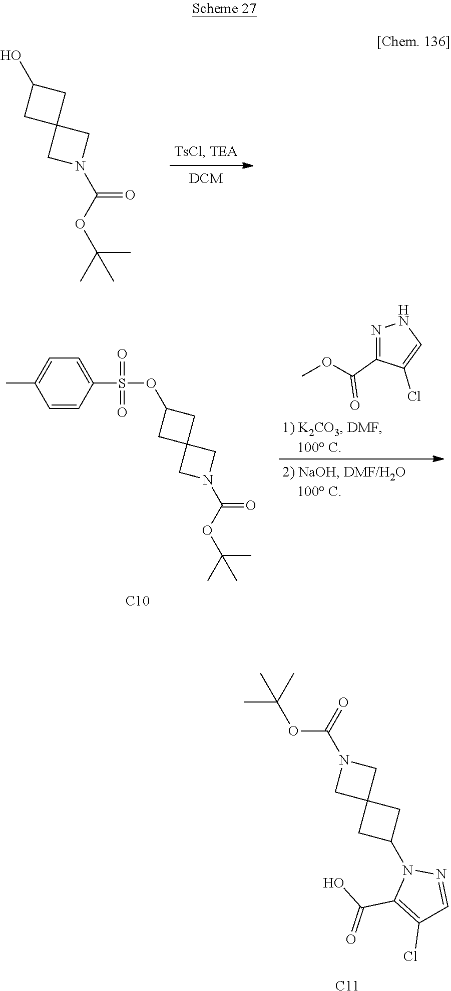

tert-butyl 6-(tosyloxy)-2-azaspiro[3.3]heptane-2-carboxylate (C10): Compounds C10 was prepared using a similar procedure described for C12. Compounds C10 was obtained as a white solid (299 mg, 87% yield). ES-MS [M+Na]⁺: 390.4, ¹H NMR (400 MHz, ((CD₃)₂SO)) δ 7.76 (d, J=8.2 Hz, 2H), 7.47 (d, J=8.2 Hz, 2H), 4.71 (p, J=7.2 Hz, 1H), 3.75 (br d, J=13.3 Hz, 4H), 2.46-2.39 (m, 5H), 2.25-2.17 (m, 2H), 1.33 (s, 9H).

1-(2-(tert-butoxycarbonyl)-2-azaspiro[3.3]heptan-6-yl)-4-chloro-1H-pyrazole-5-carb oxylic acid (C11): Compound C11 was prepared using a similar procedure described for C13. Compound C11 was obtained as a white solid that was carried forward as a TFA salt (418 mg, 26% yield over 2 steps). ES-MS [M-tBu+2H]⁺: 286.4, ¹H NMR (400 MHz, ((CD₃)₂SO)) δ 7.74 (s, 1H), 5.41 (p, J=8.0 Hz, 1H), 3.94 (s, 2H), 3.83 (s, 2H), 2.65 (d, J=8.0 Hz, 4H), 1.36 (s, 9H).

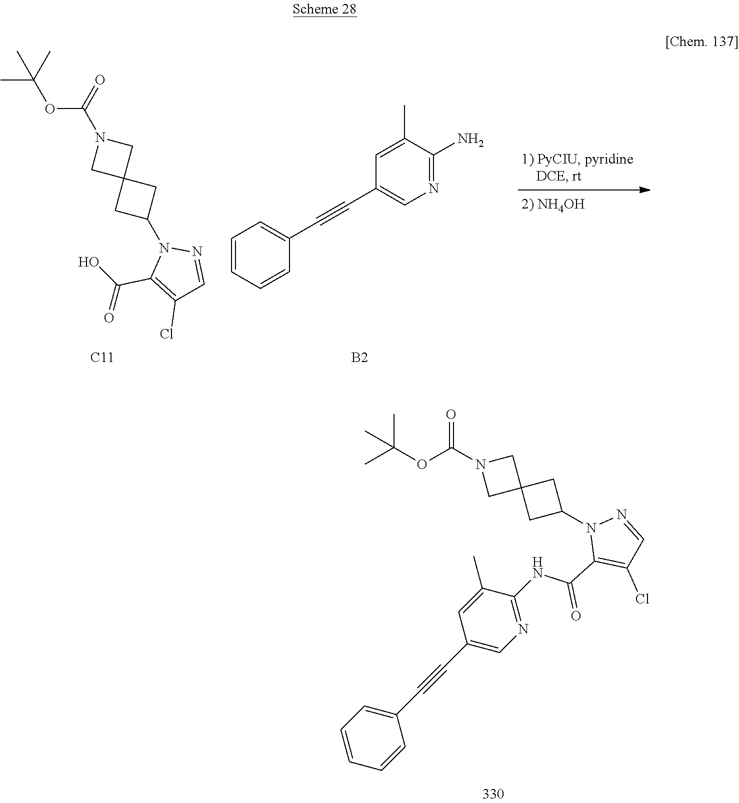

tert-butyl 6-(4-chloro-5-((3-methyl-5-(phenylethynyl)pyridin-2-yl)carbamoyl)-1H-pyrazol-1-yl)-2-azaspiro[3.3]heptane-2-carboxylate (330): Compound 330 is prepared in a similar manner as Compound 328 to yield the title compound 330 (99 mg, 63% yield). ES-MS [M+1]⁺: 532.4, LCMS Retention time: 1.16 min, ¹H NMR (400 MHz, DMSO) δ 10.91 (s, 1H), 8.40 (d, J=2.2 Hz, 1H), 7.94 (d, J=1.7 Hz, 1H), 7.71 (s, 1H), 7.62-7.51 (m, 2H), 7.49-7.40 (m, 3H), 4.98 (p, J=7.8 Hz, 1H), 3.93 (s, 2H), 3.85 (s, 2H), 2.73-2.61 (m, 2H), 2.27 (s, 3H), 1.36 (s, 9H).

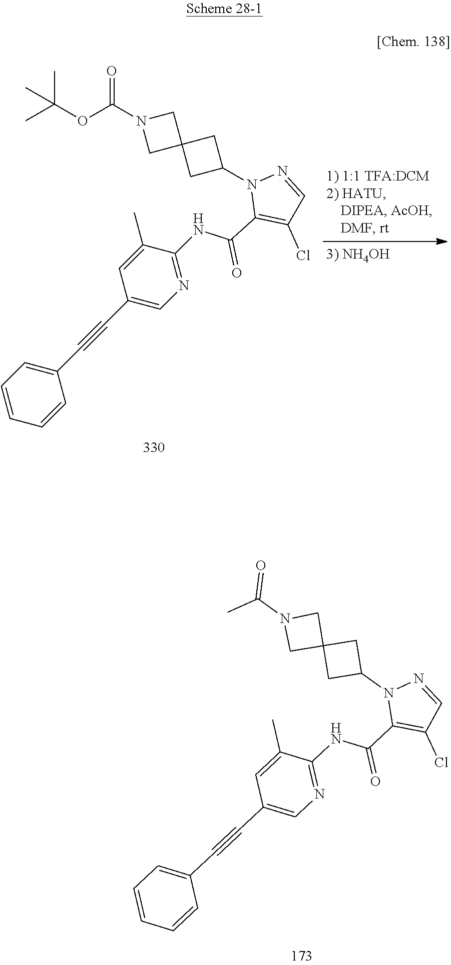 [SMILES: Cc1cc(C#Cc2ccccc2)cnc1NC(=O)c1c(Cl)cnn1C1CC2(C1)CN(C(=O)OC(C)(C)C)C2 ; CC(=O)N1CC2(CC(n3ncc(Cl)c3C(=O)Nc3ncc(C#Cc4ccccc4)cc3C)C2)C1]

1-(2-acetyl-2-azaspiro[3.3]heptan-6-yl)-4-chloro-N-(3-methyl-5-(phenylethynyl)pyridin-2-yl)-1H-pyrazole-5-carboxamide (173): Compound 173 is prepared in a similar manner as Compound 5 to yield the title compound 173 (12 mg, 52% yield). ES-MS [M+1]⁺: 474.3, LCMS Retention time: 0.99 min, ¹H NMR (400 MHz, DMSO) δ 10.92 (s, 1H), 8.40 (s, 1H), 7.94 (s, 1H), 7.71 (s, 1H), 7.63-7.52 (m, 2H), 7.48-7.40 (m, 3H), 5.08-4.96 (m, 1H), 4.16 (d, J=30.0 Hz, 2H), 3.87 (d, J=31.6 Hz, 2H), 2.75-2.64 (m, 4H), 2.27 (s, 3H), 1.72 (d, J=1.8 Hz, 3H).

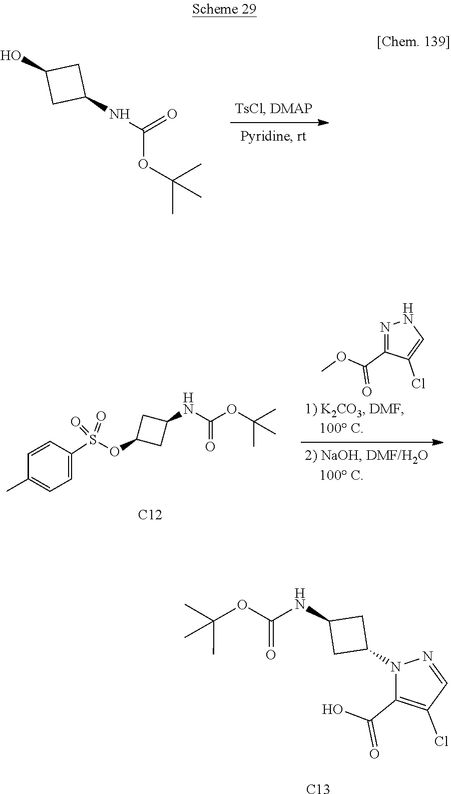

Cis-(3-(tert-butoxycarbonyl)amino)cyclobutyl 4-methylbenzenesulfonate (C12): To a solution of cis-tert-butyl (3-hydroxcyclobutyl)carbamate (2000 mg, 10.68 mmol) in pyridine (32.4 mL) was added p-toluenesulfonyl chloride (2444 mg, 12.82 mmol) and 4-dimethlaminopridine (0.15 mL, 1.07 mmol) at rt. The reaction was stirred for 24 h. The sample was concentrated in vacuo and purified by normal phase chromatography (gradient: 0-40% EtOAc in hexanes) to give C12 as a white solid (2357 mg, 65% yield). ES-MS [M+Na]⁺: 364.4, ¹H NMR (400 MHz, (CD₃)₂SO) δ 7.6 (d, J=8.2 Hz, 2H), 7.48 (d, J=8.2 Hz, 2H), 7.14 (d, J=8.1 Hz, 1H), 4.49 (p, J=7.3 Hz, 1H), 3.54 (dddd, J=16.2, 8.1, 8.1, 8.1 Hz, 1H), 2.47-2.38 (m, 5H), 2.03-1.91 (m, 2H), 1.33 (s, 9H).

1-(trans-3-((tert-butoxycarbonyl)amino)cyclobutyl)-4-chloro-1H-pyrazole-5-carboxylic acid (C13): A mixture of C12 (2357 mg, 6.9 mmol), methyl 4-chloropyrazole-3-carboxylate (2217 mg, 13.81 mmol), and potassium carbonate (2129 mg, 15.19 mmol) in DMF (10 mL) at 100° C. for 1 h. Sodium hydroxide (566 mg, 13.81 mmol) and water (5 mL) were added to the mixture, and the mixture stirred for 3.5 h at 100° C. The mixture was diluted with water and then 2 mol/L HCl was added until pH=4-5. The mixture was further diluted with water and washed with 3:1 CHCl₃:IPA. The organics were concentrated in vacuo and purified by reverse phase HPLC (gradient: 25-70% MeCN in water (w/0.1% TFA)). The desired fractions were concentrated to yield C13 as white solid that was carried forward as a TFA salt (495 mg, 23% yield over 2 steps). ES-MS [M+Na]⁺: 338.3, ¹H NMR (400 MHz, (CD₃)₂SO)) δ 7.78 (s, 1H), 7.36 (d, J=7.0 Hz, 1H), 5.59-5.49 (m, 1H), 4.21-4.09 (m, 1H), 2.72-2.62 (m, 2H), 2.46-2.37 (m, 2H), 1.39 (s, 9H).

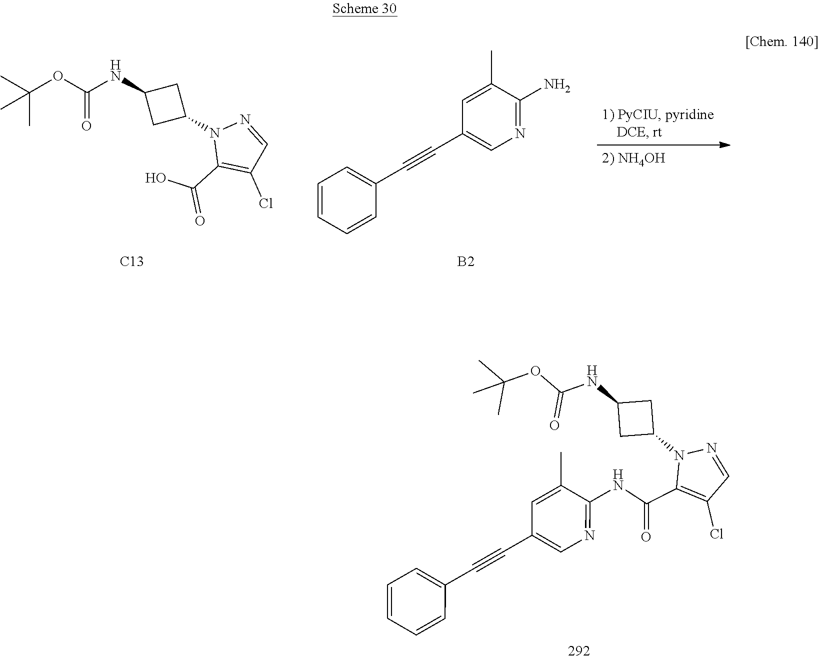 [SMILES: CC(C)(C)OC(=O)N[C@H]1C[C@H](n2ncc(Cl)c2C(=O)O)C1 ; Cc1cc(C#Cc2ccccc2)cnc1N ; "C13" ; "B2" ; Cc1cc(C#Cc2ccccc2)cnc1NC(=O)c1c(Cl)cnn1[C@H]1C[C@H](NC(=O)OC(C)(C)C)C1]

tert-butyl (trans-3-(4-chloro-5-((3-methyl-5-(phenylethynyl)pyridin-2-yl)carbamoyl)-1H-pyrazol-1-yl)cyclobutyl)carbamate (292): A mixture of C13 (200 mg, 0.465 mmol), Intermediate B2 (145 mg, 0.698 mmol), PyClU (310 mg, 0.931 mmol), and pyridine (0.380 mL, 4.65 mmol) in DCE (2.3 mL) was stirred at rt for 1 h. To the mixture was added NH₄OH (0.5 mL), and the mixture was stirred for 1. The sample was concentrated in vacuo and then purified by reverse phase HPLC (gradient: 60-95% MeCN in water (w/0.1% TFA)). The desired fractions were concentrated to yield 292 as a tan solid that was carried forward as a TFA salt (81 mg, 28% yield). ES-MS [M+1]⁺: 506.3, LCMS Retention time: 1.11 min, ¹H NMR (400 MHz, ((CD₃)₂SO)) δ 10.91 (s, 1H), 8.41 (d, J=1.6 Hz, 1H), 7.95 (dd, J=2.1, 0.6 Hz, 1H), 7.74 (s, 1H), 7.62-7.55 (m, 2H), 7.48-7.42 (m, 3H), 7.39 (br d, J=7.1 Hz, 1H), 5.21-5.11 (m, 1H), 4.22-4.11 (m, 1H), 2.78-2.68 (m, 2H), 2.46-2.37 (m, 2H), 2.28 (s, 3H), 1.38 (s, 9H).

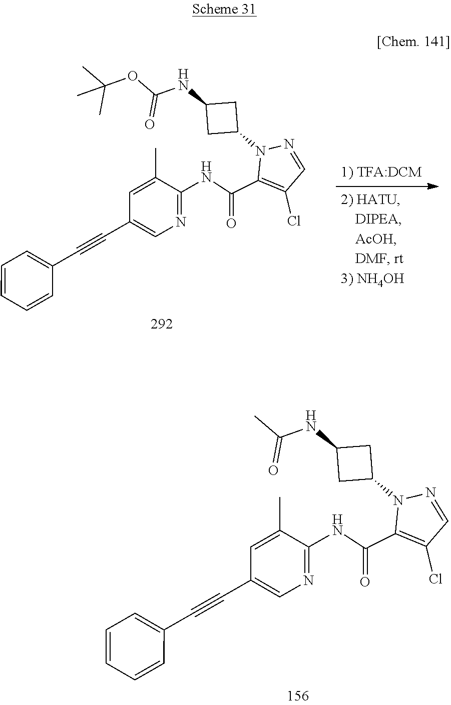 [SMILES: Cc1cc(C#Cc2ccccc2)cnc1NC(=O)c1c(Cl)cnn1[C@H]1C[C@H](NC(=O)OC(C)(C)C)C1 ; CC(=O)N[C@H]1C[C@H](n2ncc(Cl)c2C(=O)Nc2ncc(C#Cc3ccccc3)cc2C)C1]

1-(trans-3-acetamidocyclobutyl)-4-chloro-N-(3-methyl-5-(phenylethynyl)pyridin-2-yl)-1H-pyrazole-5-carboxamide (156): To the TFA salt of 292 (161 mg, 0.259 mmol) was added DCM (1.3 mL) and trifluoroacetic acid (1.3 mL, 16.98 mmol). The mixture was stirred for 1 h. The sample was concentrated in vacuo and washed with diethyl ether to give a tan solid. ES-MS [M+1]⁺: 406.2. The resulting crude amine (164 mg, 0.259 mmol) was dissolved in DMF (2.0 mL) and DIEA (0.83 mL, 4.77 mmol) was added. The mixture was stirred for 5 min and then acetic acid (0.0742 mL, 1.30 mmol) and HATU (217 mg, 0.570 mmol) were added. The mixture was stirred for 17 h. To the mixture was added NH₄OH (0.5 mL), and the mixture was stirred for 1 h. The sample was concentrated in vacuo and purified by reverse phase HPLC (gradient: 35-80% MeCN in water (w/0.05% NH₄OH)). The desired fractions were concentrated to give 156 as a white solid (73 mg, 82% over 2 steps). ES-MS [M+1]⁺: 448.3, LCMS Retention time: 1.02 min, ¹H NMR (400 MHz, (CD₃)₂SO) δ 10.92 (s, 1H), 8.40 (d, J=1.8 Hz, 1H), 8.35 (d, J=7.0 Hz, 1H), 7.93 (d, J=1.4 Hz, 1H), 7.61-7.54 (m, 2H), 7.48-7.42 (m, 3H), 5.26-5.15 (m, 1H), 4.40-4.29 (m, 1H), 2.81-2.71 (m, 2H), 2.44-2.34 (m, 2H), 2.27 (s, 3H), 1.80 (s, 3H).

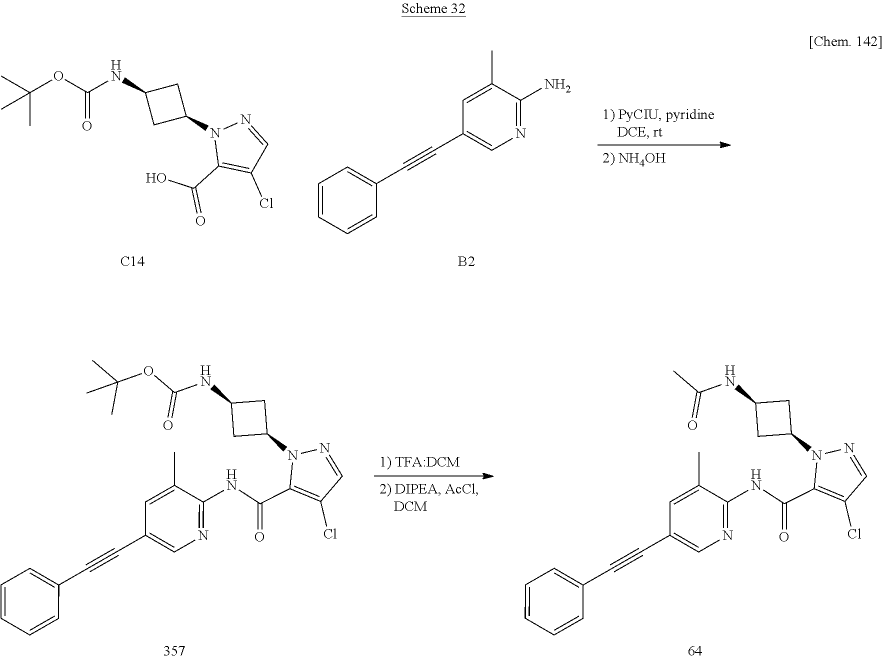 [SMILES: CC(C)(C)OC(=O)N[C@H]1C[C@@H](n2ncc(Cl)c2C(=O)O)C1 ; Cc1cc(C#Cc2ccccc2)cnc1N ; "C14" ; "B2" ; Cc1cc(C#Cc2ccccc2)cnc1NC(=O)c1c(Cl)cnn1[C@H]1C[C@@H](NC(=O)OC(C)(C)C)C1 ; CC(=O)N[C@H]1C[C@@H](n2ncc(Cl)c2C(=O)Nc2ncc(C#Cc3ccccc3)cc2C)C1]

1-(cis-3-acetamidocyclobutyl)-4-chloro-N-(3-methyl-5-(phenylethynyl)pyridin-2-yl)-1H-pyrazole-5-carboxamide (64): Intermediate (C14) was synthesized in a similar method as described in (Scheme 29). To a solution of C14 (300 mg, 0.95 mmol), Intermediate B2 (297 mg, 1.4 mmol), and PyClU (623 mg, 1.9 mmol) in DCE (6 mL) was added pyridine (752 mg, 9.5 mmol). After 3 hours at 60° C., the solution was partially concentrated and diluted with EtOAc. The dilution was washed with water 5×250 mL. The dilution was dried over anhydrous MgSO₄ and concentrated before being purified by reverse phase HPLC (gradient: 40-90% MeCN in water (water modified with 0.1% TFA)). The fractions containing product were combined and neutralized with NaHCO₃ solution and diluted with water (500 mL). The solution was extracted with EtOAc 1×200 mL. The extraction was washed with water 2×200 mL and concentrated in vacuo to yield (357) (197 mg, 41% yield). ES-MS [M+1]⁺: 506.4, LCMS Retention time: 1.10 min.

A solution of (357) (197 mg, 0.39 mmol) and TFA (1489 mg, 13.1 mmol) in DCM (5 mL) was allowed to react for 2 hours at room temperature. The solution was concentrated and the resulting crude residue diluted in DCM (5 mL) and DIEA (0.27 mL, 106 mmol). To the solution was slowly added acetyl chloride (32 mg, 0.41 mmol). After 2 hours at room temperature, the solution was purified by reverse phase HPLC (gradient: 28-80% MeCN in water (water modified with 0.1% TFA)). The fractions that contain product were combined and diluted with water and NaHCO₃ solution. The dilution was extracted with EtOAc 1×250 mL. The extraction was washed with water 3×250 mL and concentrated to yield (64) (68 mg, 39% yield). ES-MS [M+1]⁺: 448.4, LCMS Retention time: 1.01 min, ¹H NMR (400 MHz, CDCl₃) δ 8.54 (br s, 1H), 8.48 (s, 1H), 7.77 (d, J=1.3 Hz, 1H), 7.59 (s, 1H), 7.57-7.52 (m, 2H), 7.39-7.36 (m, 3H), 5.77 (d, J=7.8 Hz, 1H), 5.51 (dddd, J=8.0, 8.0, 8.0, 8.0 Hz, 1H), 4.37-4.27 (m, 1H), 3.01-2.94 (m, 2H), 2.57-2.50 (m, 2H), 2.35 (s, 3H), 1.98 (s, 3H).

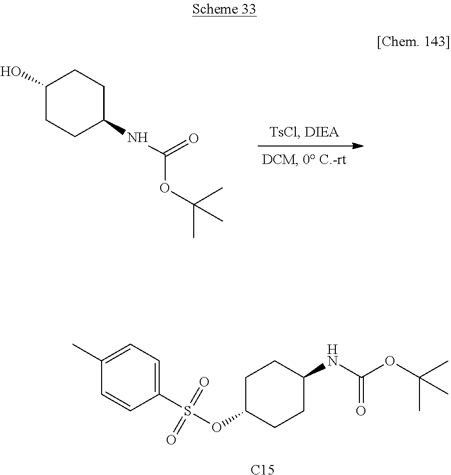 [SMILES: CC(C)(C)OC(=O)N[C@H]1CC[C@H](O)CC1 ; Cc1ccc(S(=O)(=O)O[C@H]2CC[C@H](NC(=O)OC(C)(C)C)CC2)cc1 ; "C15"]

Trans-4-((tert-butoxycarbonyl)amino)cyclohexyl 4-methylbenzenesulfonate (C15): To a solution of trans-tert-butyl (4-hydroxycyclohexyl)carbamate (5000 mg, 23.22 mmol) in DCM (40.3 mL) was added p-toluenesulfonyl chloride (5313 mg, 27.87 mmol) and TEA (6.7 mL, 47.72 mmol) at 0° C. The reaction was warmed to rt and stirred for 17 h. The sample was concentrated in vacuo and purified by normal phase chromatography (gradient: 0-40% EtOAc in hexanes) to give C15 as an off-white solid (3523 mg, 41% yield over 2 steps). ES-MS [M+Na]⁺: 392.4, ¹H NMR (400 MHz, (CD₃)₂SO) δ 7.79 (d, J=8.2 Hz, 2H), 7.47 (d, J=8.2 Hz, 2H), 6.72 (d, J=7.4 Hz, 1H), 4.39-4.29 (m, 1H), 3.3.26-3.14 m, 1H), 2.42 (s, 3H), 1.81-1.66 (m, 4H), 1.53-1.39 (m, 2H), 1.35 (s, 9H), 1.25-1.10 (m, 2H).

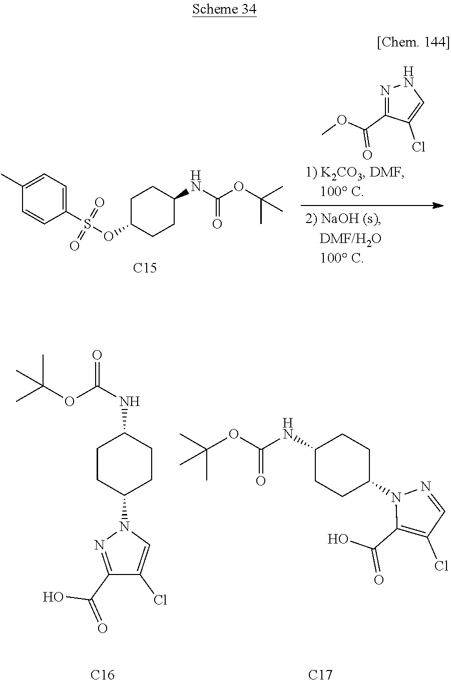

1-((cis-4-((tert-butoxycarbonyl)amino)cyclohexyl)-4-chloro-1H-pyrazole-3-carboxyli c acid (C16) and 1-(cis-4-((tert-butoxycarbonyl)amino)cyclohexyl)-4-chloro-1H-pyrazole-5-carboxylic acid (C17): Compounds C16 and C17 were prepared using a similar procedure described for C13. Compounds C16 and C17 were separated by reverse phase HPLC (gradient: 30-65% MeCN in water (w/0.1% TFA)). Compound C16 was obtained as an off-white solid that was carried forward as a TFA salt salt (26 mg, 8% yield over 2 steps). ES-MS [M+Na]⁺: 366.4, ¹H NMR (400 MHz, ((CD₃)₂SO)) δ 8.19 (s, 1H), 6.90 (d, J=6.9 Hz, 1H), 4.26-4.16 (m, 1H), 3.60 (br s, 1H), 2.15-2.02 (m, 2H), 1.83-1.73 (m, 2H), 1.71-1.54 (m, 4H), 1.39 (s, 9H). Compound C17 was obtained as a white solid that was carried forward as a TFA salt (316 mg, 44% yield over 2 steps). ES-MS [M-tBu+2H]⁺: 288.4, ¹H NMR (400 MHz, ((CD₃)₂SO)) δ 7.71 (s, 1H), 6.90 (br s, 1H), 5.01-4.90 (m, 1H), 3.59-3.51 (m, 1H), 2.15-2.00 (m, 2H), 1.89-1.79 (m, 2H), 1.72-1.63 (m, 2H), 1.60-1.49 (m, 2H), 1.39 (s, 9H).

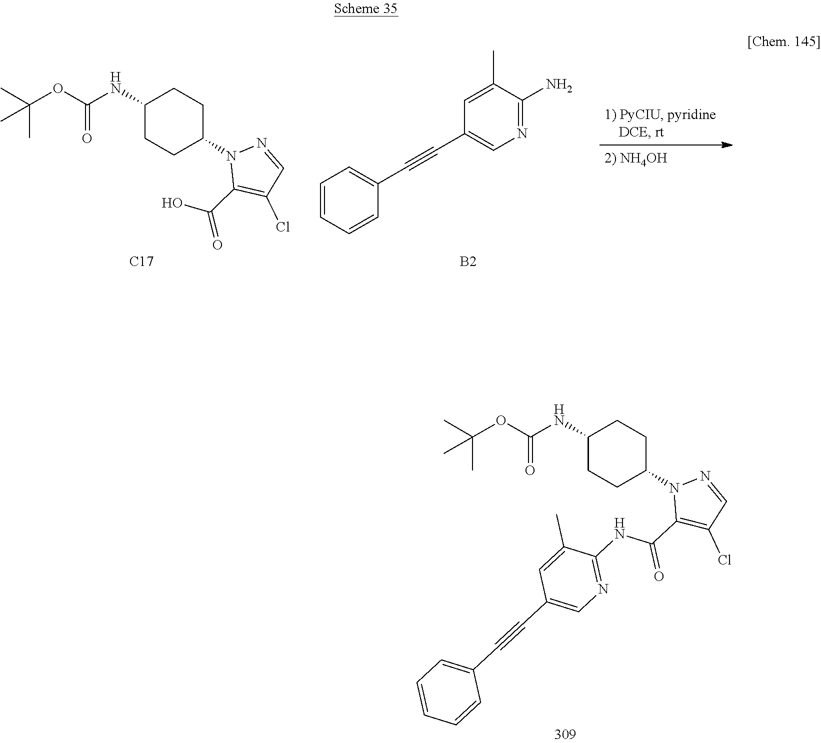

Tert-butyl (cis-4-(4-chloro-5-((3-methyl-5-(phenylethynyl)pyridin-2-yl)carbamoyl)-1H-pyrazol-1-yl)cyclohexyl)carbamate (309): Compound 309 was prepared using a similar procedure described for 328. Compound 309 was obtained as a brown solid that was carried forward as a TFA salt (654 mg, 50% yield). ES-MS [M+1]⁺: 534.2, LCMS Retention time: 1.27 min, ¹H NMR (400 MHz, (CD₃)₂SO)) δ 10.96 (s, 1H), 8.41 (d, J=1.7 Hz, 1H), 7.95 (dd, J=2.0, 0.7 Hz, 1H), 7.65 (s, 1H), 7.65-7.58 (m, 2H), 7.48-7.43 (m, 3H), 6.91 (br s, 1H), 4.54-4.42 (m, 1H), 3.57-3.49 (m, 1H), 2.28 (s, 3H), 2.22-2.09 (m, 2H), 1.90-1.80 (m, 2H), 1.80-1.70 (m, 2H), 1.62-1.50 (m, 2H), 1.40 (s, 9H).

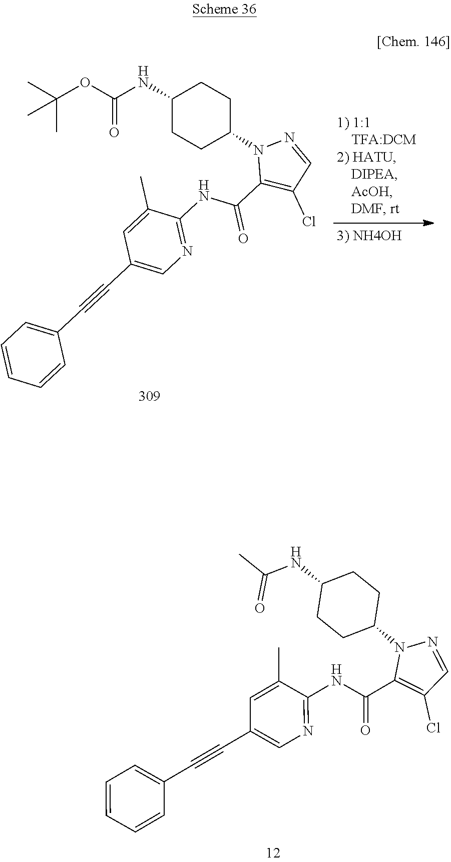 [SMILES: Cc1cc(C#Cc2ccccc2)cnc1NC(=O)c1c(Cl)cnn1[C@H]1CC[C@@H](NC(=O)OC(C)(C)C)CC1 ; CC(=O)N[C@H]1CC[C@@H](n2ncc(Cl)c2C(=O)Nc2ncc(C#Cc3ccccc3)cc2C)CC1]

1-(cis-4-acetamidocyclohexyl)-4-chloro-N-(3-methyl-5-(phenylethynyl)pyridin-2-yl)-1H-pyrazole-5-carboxamide (12): Compound 12 was prepared using a similar procedure described for 5. Compound 12 was obtained as an off-white solid (349 mg, 98% yield over 2 steps). ES-MS [M+1]⁺: 476.3, LCMS Retention time: 1.08 min, ¹H NMR (400 MHz, (CD₃)₂SO) δ 10.96 (s, 1H), 8.40 (d, J=1.5 Hz, 1H), 7.94 (d, J=1.5 Hz, 1H), 7.92 (d, J=6.8 Hz, 1H), 7.66 (s, 1H), 7.62-7.55 (m, 2H), 7.48-7.42 (m, 3H), 4.54-4.44 (m, 1H), 3.86-3.78 (m, 1H), 2.28 (s, 3H), 2.21-2.08 (m, 2H), 1.85 (s, 3H), 1.83-1.76 (m, 4H), 1.65-1.53 (m, 2H).

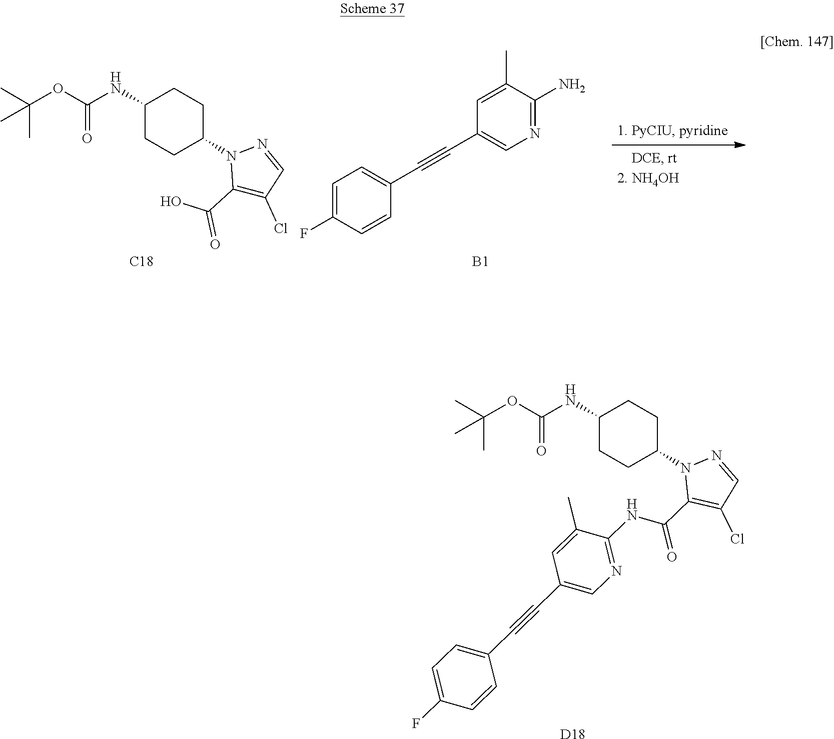

tert-butyl (cis-4-(4-chloro-5-((5-((4-fluorophenyl)ethynyl)-3-methylpyridin-2-yl)carbamoyl)-1H-pyrazol-1-yl)cyclohexyl)carbamate (D18): Compound D18 was prepared using a similar procedure described for 328. Compound D18 was obtained as a brown solid that was carried forward as a TFA salt (43 mg, 44% yield). ES-MS [M+1]⁺: 552.4, ¹H NMR (400 MHz, CDCl₃) δ 8.48 (d, J=1.9 Hz, 1H), 7.77 (dd, J=2.0, 0.7 Hz, 1H), 7.56 (s, 1H), 7.55-7.50 (m, 2H), 7.07 (dd, J=8.7, 8.7 Hz, 2H), 5.24-5.14 (m, 1H), 4.87 (d, J=5.5 Hz, 1H), 3.86 (s, 3H), 2.36 (s, 3H), 2.15-1.99 (m, 2H), 1.94 (br d, J=11.0, 4H), 1.77-1.64 (m, 2H), 1.45 (s, 9H).

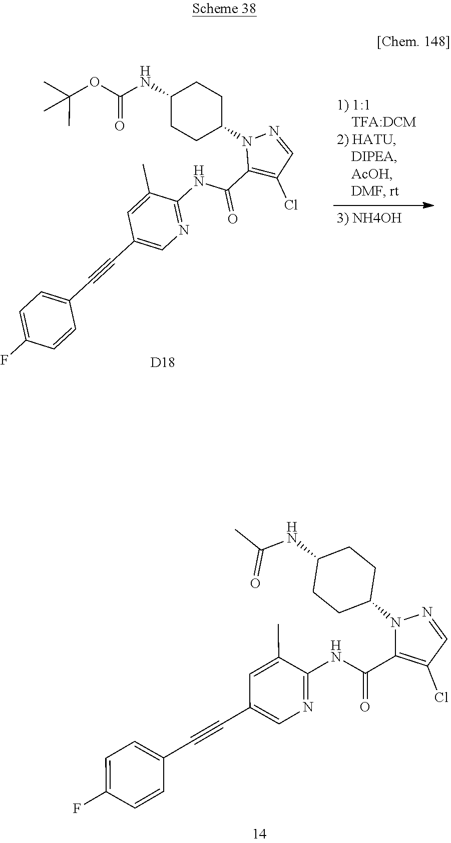 [SMILES: Cc1cc(C#Cc2ccc(F)cc2)cnc1NC(=O)c1c(Cl)cnn1[C@H]1CC[C@@H](NC(=O)OC(C)(C)C)CC1 ; "D18" ; CC(=O)N[C@H]1CC[C@@H](n2ncc(Cl)c2C(=O)Nc2ncc(C#Cc3ccc(F)cc3)cc2C)CC1]

1-(cis-4-acetamidocyclohexyl)-4-chloro-N-(5-((4-fluorophenyl)ethynyl)-3-methylpyridin-2-yl)-1H-pyrazole-5-carboxamide (14): Compound 14 was prepared using a similar procedure described for ESC Sch5 Comp14. Compound 14 was obtained as an off-white solid (107 mg, 98% yield over 2 steps). ES-MS [M+1]⁺: 494.3, LCMS Retention time: 1.11 min, ¹H NMR (400 MHz, (CD₃)₂SO) δ 10.96 (s, 1H), 8.40 (d, J=1.5 Hz, 1H), 7.95-7.88 (m, 2H), 7.68-7.60 (m, 3H), 7.33-7.26 (m, 2H), 4.55-4.43 (m, 1H), 3.86-3.78 (m, 1H), 2.28 (s, 3H), 2.20-2.08 (m, 2H), 1.84 (s, 3H), 1.83-1.74 (m, 4H), 1.64-1.52 (m, 2H).

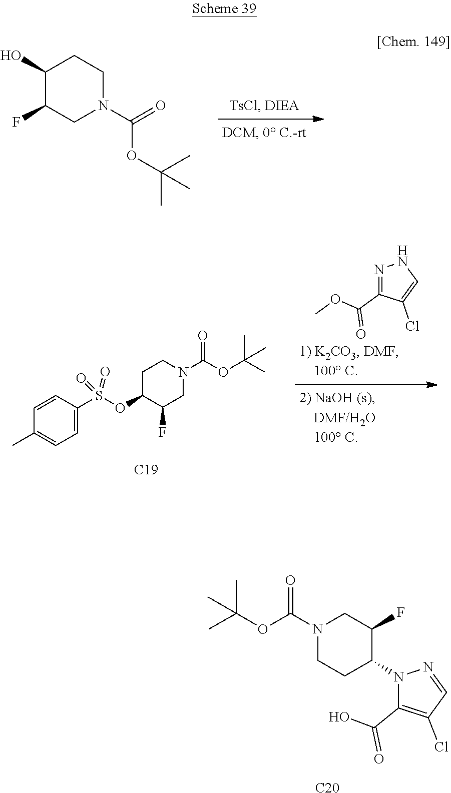

tert-butyl (3R,4S)-3-fluoro-4-(tosyloxy)piperidine-1-carboxylate (C19): Compound C19 was prepared using a similar procedure described for C12. Compounds C19 was obtained as an off-white solid (1577 mg, 93%). ES-MS [M+Na]⁺: 396.3, ¹H NMR (400 MHz, (CD₃)₂SO)) δ 7.83 (d, J=8.2 Hz, 2H), 7.49 (d, J=8.2 Hz, 2H), 4.88-4.78 (m, 1H), 4.78-4.62 (m, 1H), 4.04-3.92 (m, 1H), 3.89-3.64 (br s, 1H), 3.28-3.07 (m, 1H), 3.07-2.80 (m, 1H), 2.43 (s, 3H), 1.80-1.60 (m, 1H), 1.65-1.55 (m, 1H), 1.36 (s, 9H).

1-((3R,4R)-1-(tert-butoxycarbonyl)-3-fluoropiperidin-4-yl)-4-chloro-1H-pyrazole-5-carboxylic acid (C20): Compound C20 was prepared using a similar procedure described for C13. Compound C20 was obtained as an off-white solid that was carried forward as a TFA salt (384 mg, 20% yield over 2 steps). ES-MS [M-tBu+2H]⁺: 292.1, ¹H NMR (400 MHz, (CD₃)₂SO)) δ 7.85 (s, 1H), 5.51-5.38 (m, 1H), 4.91-4.68 (m, 1H), 4.26 (br s, 1H), 3.96 (d, J=12.2 Hz, 1H), 2.98 (br s, 2H), 2.11-2.02 (m, 1H), 1.97-1.81 (m, 1H), 1.42 (s, 9H).

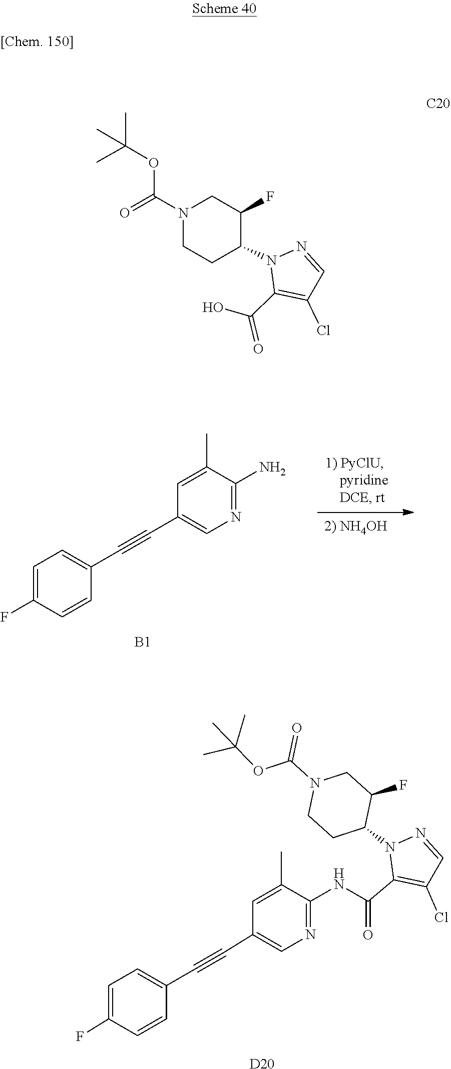 [SMILES: CC(C)(C)OC(=O)N1CC[C@@H](n2ncc(Cl)c2C(=O)O)[C@H](F)C1 ; Cc1cc(C#Cc2ccc(F)cc2)cnc1N ; "B1" ; Cc1cc(C#Cc2ccc(F)cc2)cnc1NC(=O)c1c(Cl)cnn1[C@@H]1CCN(C(=O)OC(C)(C)C)C[C@H]1F ; "D20"]

tert-butyl (3R,4R)-4-(4-chloro-5-((5-((4-fluorophenyl)ethynyl)-3-methylpyridin-2-yl)carbamoyl)-1H-pyrazol-1-yl)-3-fluoropiperidine-1-carboxylate (D20): Compound D20 was prepared using a similar procedure described for 328. Compound D20 was obtained as off-white solid that was carried forward as a TFA salt (71 mg, 74% yield). ES-MS [M+1]⁺: 556.3, ¹H NMR (400 MHz, (CD₃)₂SO)) δ 11.03 (s, 1H), 8.44 (d, J=1.6 Hz, 1H), 7.95 (d, J=1.6 Hz, 1H), 7.81 (s, 1H), 7.69-7.61 (m, 2H), 7.34-7.26 (m, 2H), 4.93-4.69 (m, 2H), 4.28 (br s, 1H), 3.96 (d, J=10.6 Hz, 1H), 2.99 (br s, 2H), 2.27 (s, 3H), 2.18-2.08 (m, 1H), 2.03-1.87 (m, 1H), 1.42 (s, 9H).

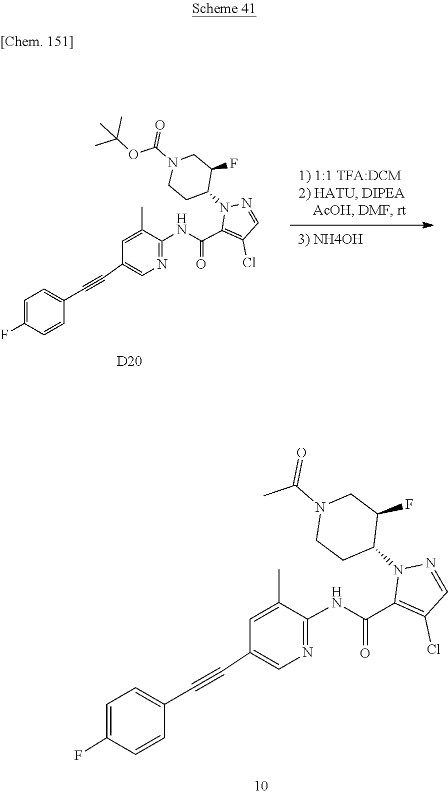 [SMILES: Cc1cc(C#Cc2ccc(F)cc2)cnc1NC(=O)c1c(Cl)cnn1[C@@H]1CCN(C(=O)OC(C)(C)C)C[C@H]1F ; CC(=O)N1CC[C@@H](n2ncc(Cl)c2C(=O)Nc2ncc(C#Cc3ccc(F)cc3)cc2C)[C@H](F)C1 ; "D20"]

1-((3R,4R)-1-acetyl-3-fluoropiperidin-4-yl)-4-chloro-N-(5-((4-fluorophenyl)ethynyl)-3-methylpyridin-2-yl)-1H-pyrazole-5-carboxamide (10): Compound 10 was prepared using a similar procedure described for 5. Compound 10 was obtained as an off-white solid (1063 mg, 95% yield over 3 steps). ES-MS [M+1]⁺: 498.3, LCMS Retention time: 0.99 min, ¹H NMR (400 MHz, (CD₃)₂SO) δ 11.04 (s, 1H), 8.46 (d, J=1.6 Hz, 1H), 7.96 (d, J=1.6 Hz, 1H), 7.82 (s, 1H), 7.70-7.62 (m, 2H), 7.36-7.28 (m, 2H), 5.12-4.77 (m, 2H), 4.76-4.33 (m, 1H), 4.26-3.82 (m, 1H), 3.31-3.18 (m, 1H), 2.94-2.71 (m, 1H), 2.28 (s, 3H), 2.21-1.82 (m, 5H).

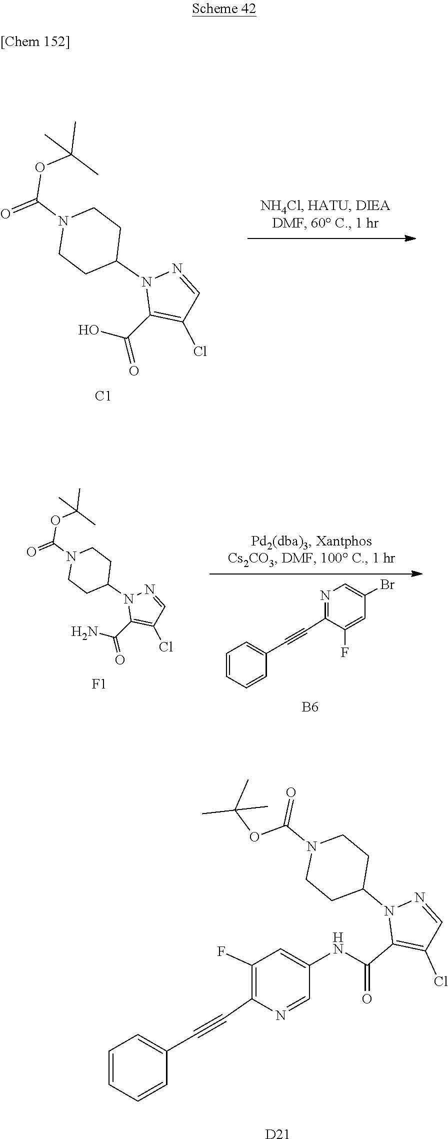

tert-butyl 4-(4-chloro-5-((5-fluoro-6-(phenylethynyl)pyridin-3-yl)carbamoyl)-1H-pyrazol-1-yl)piperidine-1-carboxylate (D21): To a solution of (C1) (150 mg, 0.45 mmol), NH₄Cl (24 mg, 0.45 mmol), and HATU (259 mg, 0.68 mmol) in DMF (2.5 mL) was added DIEA (176 mg, 1.36 mmol). After 1 hour at 60° C., the solution was purified by revers phase HPLC (gradient: 8-95% MeCN/water (w/0.05% NH₄OH)). After removal of solvent, (F1) was obtained (61 mg, 41% yield). ES-MS [M+23]⁺: 351.2.

Intermediate B6 was prepared in a method similar to that described in Intermediate B1. To a dry microwave vial was added (F1) (30 mg, 0.09 mmol), (Intermediate B6) (25 mg, 0.09 mmol), Pd₂(dba)₃ (5 mg, 0.005 mmol), xantphos (3 mg, 0.005 mmol), and Cs₂CO₃ (60 mg, 0.18 mmol). The vial was sealed and evacuated and backfilled with dry nitrogen 3×. To the vial was added DMF (1.2 mL). After 1 hour at 100° C., the solution was purified by reverse phase HPLC (gradient: 30-95% MeCN/water (w/0.05% NH₄OH)). Fractions that contained isolated product were concentrated to yield (D21) (32 mg, 66% yield).

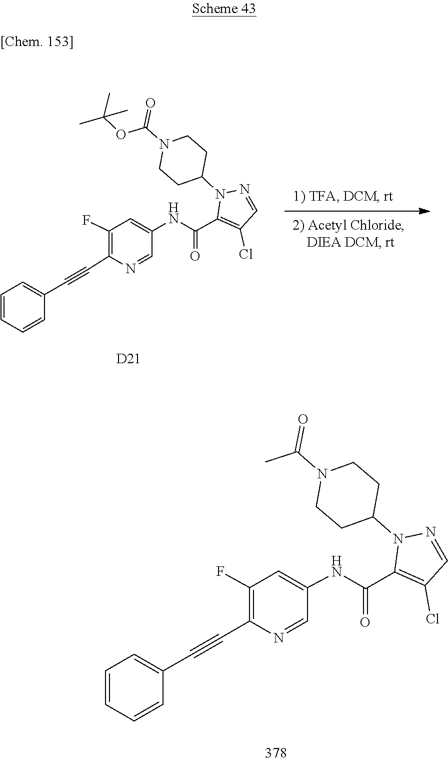 [SMILES: CC(C)(C)OC(=O)N1CCC(n2ncc(Cl)c2C(=O)Nc2cnc(C#Cc3ccccc3)c(F)c2)CC1 ; "D21" ; CC(=O)N1CCC(n2ncc(Cl)c2C(=O)Nc2cnc(C#Cc3ccccc3)c(F)c2)CC1]

1-(1-acetylpiperidin-4-yl)-4-chloro-N-(5-fluoro-6-(phenylethynyl)pyridin-3-yl)-1H-pyrazole-5-carboxamide (Compound 378): A solution of (378) (32.5 mg, 0.06 mmol) and DCM:TFA 1:1 (2 mL) was allowed to reacted for 15 minutes at room temperature. The solution was concentrated and the resulting crude residue was dissolved in DMF (1.1 mL), DIEA (39 mg, 0.30 mmol), and acetyl chloride (7.1 mg, 0.09 mmol) and was allowed to react at room temperature. After 18 hours, the solution was purified by reverse phase HPLC (gradient: 30-95% MeCN/water (w/0.05% NH₄OH)). The fractions that contained isolated product were concentrated to yield (378) (20 mg, 69% yield). ES-MS [M+1]⁺: 466.0, LCMS Retention time: 1.06 min.

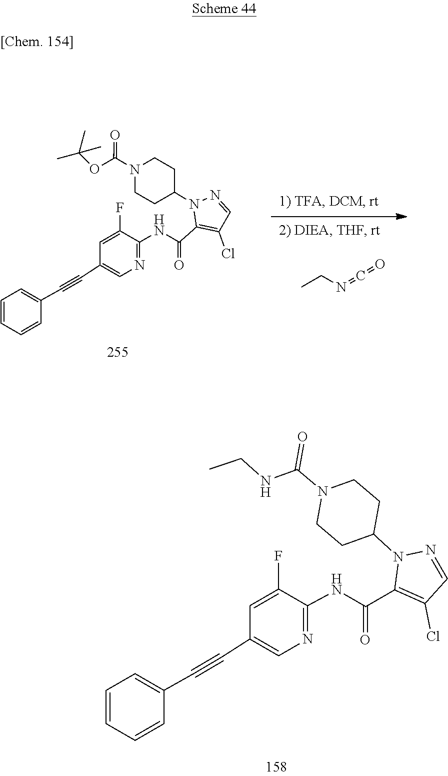

4-(4-chloro-5-((3-fluoro-5-(phenylethynyl)pyridin-2-yl)carbamoyl)-1H-pyrazol-1-yl)-N-ethylpiperidine-1-carboxamide (158): A solution of tert-butyl 4-(4-chloro-5-((3-fluoro-5-(phenylethynyl)pyridin-2-yl)carbamoyl)-1H-pyrazol-1-yl)piperidine-1-carboxylate 255 (15 mg, 0.03 mmol) and DCM:TFA 1:1 (2 mL) was stirred at rt. After 2 h, the solution was concentrated and the resulting crude residue was dissolved in THF (0.5 mL) and DIEA (18 mg, 0.14 mmol) and isocyanatoethane (0.03 mmol) was added. After 2 hours at rt, the solution was purified by reverse phase HPLC (gradient: 40-95% MeCN/water (w/0.05% NH₄OH)) to afford (158) (3 mg, 22% yield). ES-MS [M+1]⁺: 495.3, LCMS Retention time: 0.99 min.

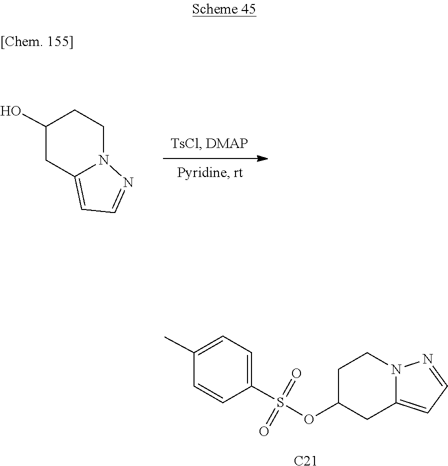 [SMILES: OC1CCn2nccc2C1 ; Cc1ccc(S(=O)(=O)OC2CCn3nccc3C2)cc1 ; "C21"]

4,5,6,7-tetrahydropyrazolo[1,5-a]pyridin-5-yl 4-methylbenzenesulfonate (C21): Compound C21 was prepared using a similar procedure described for C12. Compounds C21 was obtained as an off-white solid (525 mg, 62% yield). ES-MS [M+H]⁺: 293.4, ¹H NMR (400 MHz, ((CD₃)₂SO)) δ 7.84 (d, J=8.2 Hz, 2H), 7.50 (d, J=8.2 Hz, 2H), 7.36 (d, J=1.8 Hz, 1H), 6.01-5.99 (m, 1H), 5.10-5.04 (m, 1H), 4.20-4.10 (m, 1H), 4.05-3.94 (m, 1H), 3.04 (dd, J=17.1, 4.4 Hz, 1H), 2.91 (dd, J=17.1, 4.4 Hz, 1H), 2.43 (s, 3H), 2.22-2.07 (m, 2H), 1.39.

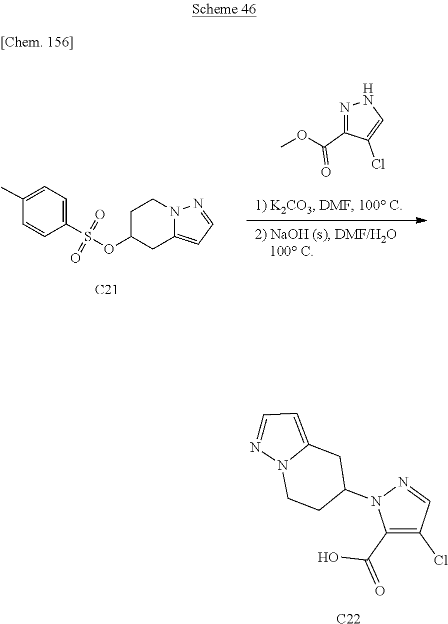

4-chloro-1-(4,5,6,7-tetrahydropyrazolo[1,5-a]pyridin-5-yl)-1H-pyrazole-5-carboxylic acid (C22): Compounds C22 was prepared using a similar procedure described for C13. Compound C22 was obtained as an off-white solid that was carried forward as a TFA salt (81 mg, 17% yield over 2 steps). ES-MS [M+H]⁺: 267.4, ¹H NMR (400 MHz, (CD₃)₂SO)) δ 7.77 (s, 1H), 7.39 (d, J=1.8 Hz, 1H), 6.04 (d, J=1.8 Hz 1H), 5.61-5.52 (m, 1H), 4.18-4.11 (m, 2H), 3.30 (br dd, J=16.1 Hz, 5.3 Hz, 1H), 3.14 (br dd, J=16.1, 8.8 Hz, 1H), 2.57-2.51 (m, 1H), 2.40-2.31 (m, 1H).

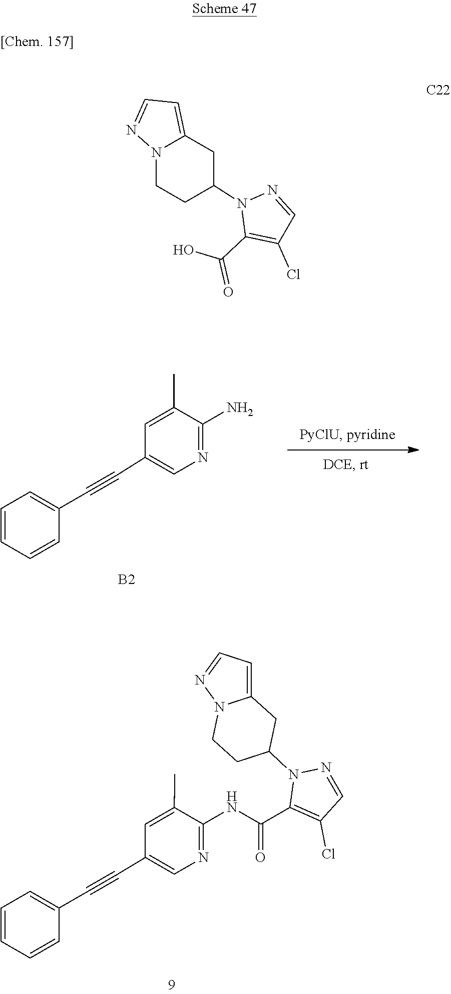 [SMILES: O=C(O)c1c(Cl)cnn1C1CCn2nccc2C1 ; Cc1cc(C#Cc2ccccc2)cnc1N ; "B2" ; Cc1cc(C#Cc2ccccc2)cnc1NC(=O)c1c(Cl)cnn1C1CCn2nccc2C1]

4-chloro-N-(3-methyl-5-(phenylethynyl)pyridin-2-yl)-1-(4,5,6,7-tetrahydropyrazolo [1,5-a]pyridin-5-yl)-1H-pyrazole-5-carboxamide (9): Compound 9 was prepared using a similar procedure described for 328. Compound 9 was obtained as an off-white solid (4 mg, 22% yield). ES-MS [M+1]⁺: 457.4, LCMS Retention time: 1.06 min, ¹H NMR (400 MHz, ((CD₃)₂SO)) δ 11.02 (s, 1H), 8.40 (d, J=1.7 Hz, 1H), 7.94 (dd, J=2.1, 0.7 Hz, 1H), 7.70 (s, 1H), 7.61-7.55 (m, 2H), 7.48-7.43 (m, 3H), 7.39 (d, J=1.7 Hz, 1H), 6.06 (d, J=1.7 Hz, 1H), 5.08-5.07 (m, 1H), 4.23-4.14 (m, 1H), 3.41-3.33 (m, 1H), 3.31-3.20 (m, 2H), 1.90-1.80 (m, 1H), 2.63-2.51 (m, 1H), 2.45-2.36 (m, 1H), 2.30 (s, 3H).

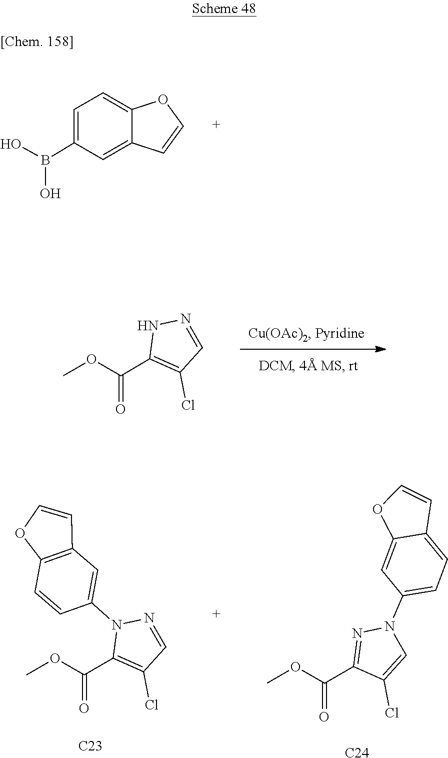

Methyl 1-(benzofuran-5-yl)-4-chloro-1H-pyrazole-5-carboxylate (C23) and methyl 1-(benzofuran-6-yl)-4-chloro-1H-pyrazole-5-carboxylate (C24). To a solution of benzofuran-5-ylboronic acid (100 mg, 0.63 mmol) and methyl 4-chloro-1H-pyrazole-5-carboxylate (50 mg, 0.31 mmol) in DCM (2.5 mL) was added copper (II) acetate (85 mg, 0.47 mmol), pyridine (0.050 mL, 0.62 mmol) and 150 mg 4 Å molecular sieves. The vial was loosely capped to allow air to enter and after 16 h at rt, the reaction was filtered through celite, washed with methanol and concentrated in vacuo. The resulting residue was purified by normal phase chromatography (0-40% ethyl acetate in hexanes). Fractions were concentrated to yield an inseparable mixture of (C23) and (C24) (17 mg, 20% yield). ES-MS [M+1]⁺: 277.2.

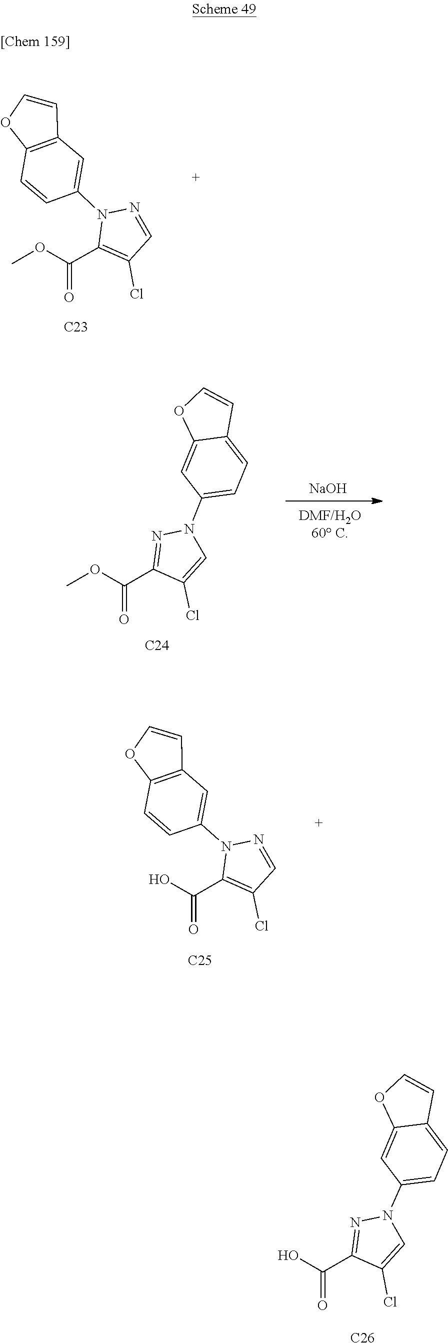

1-(benzofuran-5-yl)-4-chloro-1H-pyrazole-5-carboxylic acid (C25) and 1-(benzofuran-6-yl)-4-chloro-1H-pyrazole-3-carboxylic acid (C26). To a solution of the mixture of (C23) and (C24) (17 mg, 0.061 mmol) in DMF (0.5 mL) and water (0.5 mL) was added NaOH (5 mg, 0.12 mmol). The reaction mixture was heated to 60° C. After 30 min, the reaction was cooled to room temperature and acidified (pH ˜4-5) with 2 mol/L HCl. After the mixture was extracted with DCM (3×), the combined organic layers were passed through a phase separator and concentrated to afford an inseparable mixture of (C25) and (C26) (16 mg, 99% yield). ES-MS [M+1]⁺: 263.2.

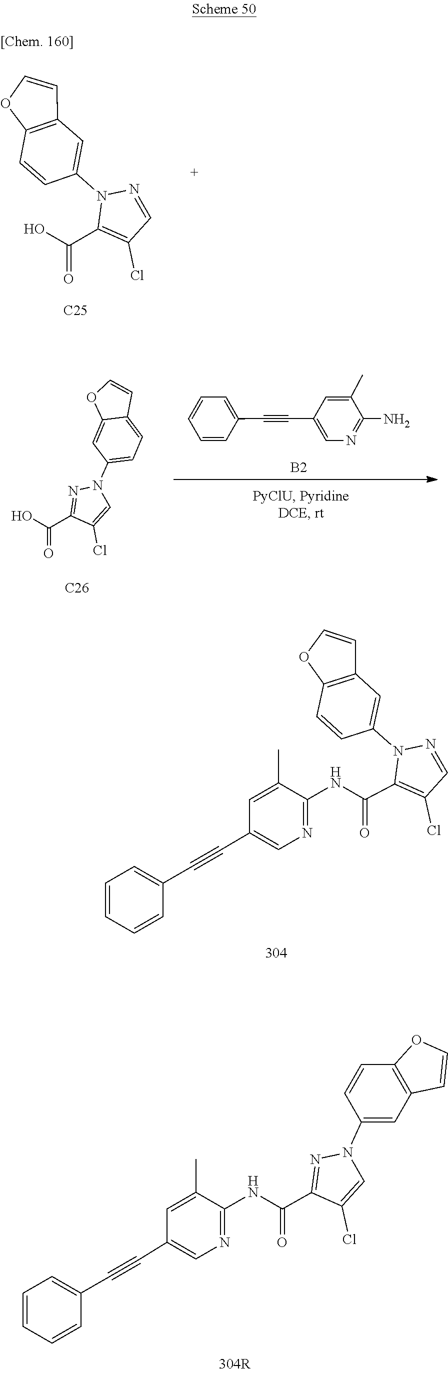

1-(benzofuran-5-yl)-4-chloro-N-(3-methyl-5-(phenylethynyl)pyridin-2-yl)-1H-pyrazole-5-carboxamide (Compound 304). To a solution of the mixture of (C25) and (C26) (16 mg, 0.06 mmol) in DCE (1 mL) was added Intermediate B2 (19 mg, 0.09 mmol), PyClU (41 mg, 0.12 mmol) and pyridine (0.049 mL, 0.06 mmol). The reaction stirred for 16 hours at room temperature. After filtering the reaction, the solution was purified by reverse phase HPLC (gradient: 40-85% MeCN/water (w/0.1% TFA)). The combined fractions containing product were basified with saturated NaHCO₃ and concentrated, followed by dilution with water and extraction with DCM (2×). The combined organic layers were concentrated to yield (304) (2.3 mg, 8% yield) and (304R) (10 mg, 36% yield). NOSEY was used to identify (304) as desired regioisomer (eluted first on reverse phase HPLC). (304): ES-MS [M+1]⁺: 453.3, LCMS Retention time: 1.09 min, ¹H NMR (400 MHz, CD₃OD) δ 8.21 (br, 1H), 7.88 (br, 1H), 7.78 (d, J=2 Hz, 1H), 7.72 (d, J=2.1 Hz, 1H), 7.65 (br, 2H), 7.51 (d, J=8.8 Hz, 1H), 7.44-7.41 (m, 3H), 7.30-7.29 (m, 2H), 6.83 (dd, J=2.2, 0.9 Hz, 1H), 2.05 (s, 3H). (304R): ES-MS [M+1]⁺: 453.2, ¹H NMR (400 MHz, DMSO-d₆) δ 10.62 (br, 1H), 9.02 (br, 1H), 8.58 (d, J=1.8 Hz, 1H), 8.34 (d, J=2.3 Hz, 1H), 8.21 (d, J=2.2 Hz, 1H), 8.04-8.02 (m 2H), 7.87 (d, J=8.9 Hz), 7.67-7.65 (m, 2H), 7.54-7.51 (m, 3H), 7.15 (dd, J=2.2, 0.8 Hz, 1H), 2.35 (s, 3H).

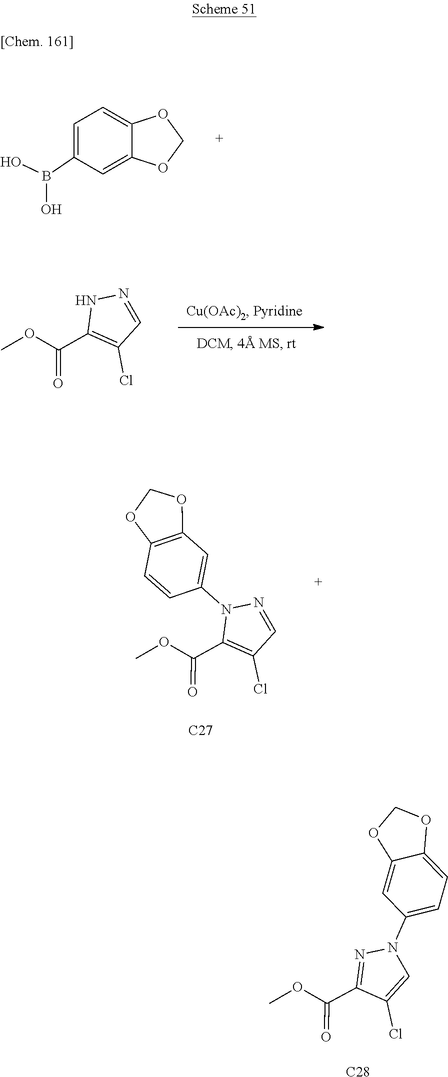

methyl 1-(benzo[d][1,3]dioxol-5-yl)-4-chloro-1H-pyrazole-5-carboxylate (C27) and methyl 1-(benzo[d][1,3]dioxol-5-yl)-4-chloro-1H-pyrazole-3-carboxylate (C28). Intermediates C27 and C28 were prepared in a similar fashion to C23 and C24. The compounds were obtained as an inseparable mixture of (C27) and (C28) (77 mg, 88% yield). ES-MS [M+1]⁺: 281.1.

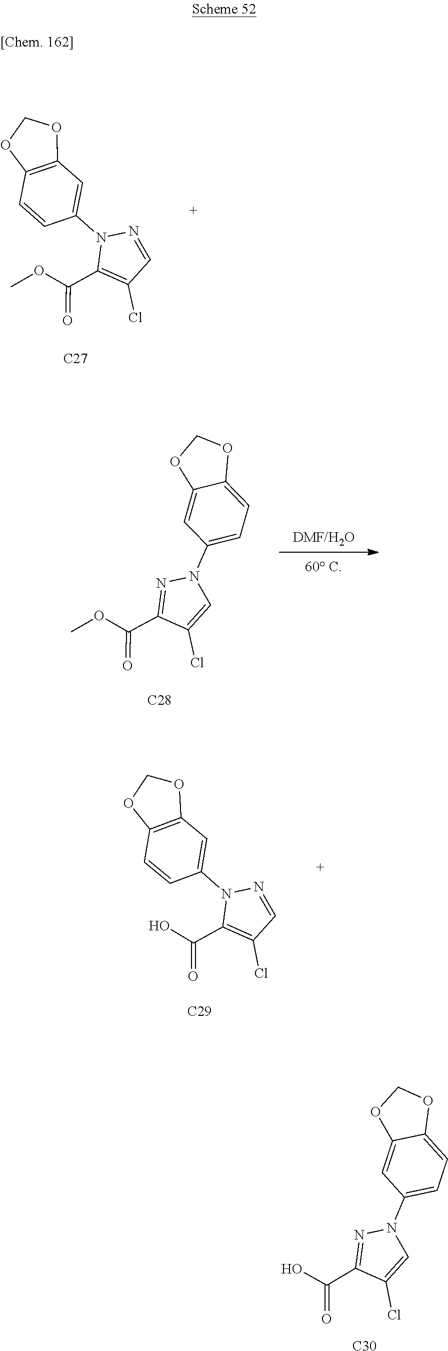

1-(benzo[d][1,3]dioxol-5-yl)-4-chloro-1H-pyrazole-5-carboxylic acid (C29) and 1-(benzo[d][1,3]dioxol-5-yl)-4-chloro-1H-pyrazole-3-carboxylic acid (C30). Intermediates C29 and C30 were prepared in a similar fashion to C25 and C26. The compounds were obtained as an inseparable mixture (77 mg) and the crude product was carried forward without further purification. ES-MS [M+1]⁺: 267.2.

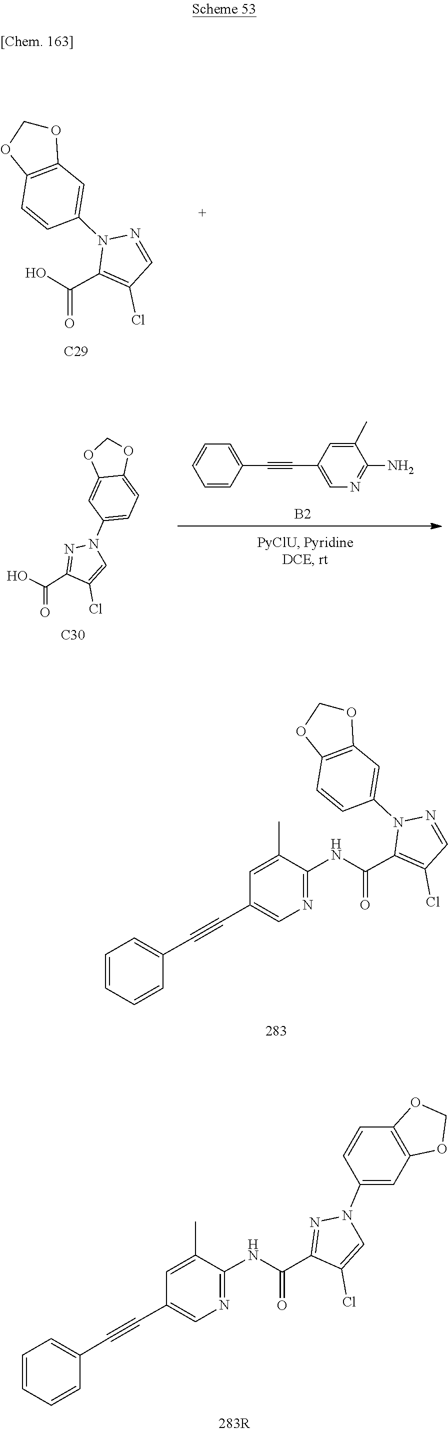

1-(benzol[d][1,3]dioxol-5-yl)-4-chloro-N-(3-methyl-5-(phenylethynyl)pyridin-2-yl)-1H-pyrazole-5-carboxamide (283). To a solution of the mixture of (C29) and (C30) (33 mg, 0.124 mmol) in DCE (1.8 mL) was added Intermediate B2 (39 mg, 0.19 mmol), PyClU (82 mg, 0.25 mmol) and pyridine (0.10 mL, 1.24 mmol). The reaction stirred for 16 hours at room temperature. After filtering the reaction, the solution was purified by reverse phase HPLC (gradient: 45-80% MeCN in water (0.1% TFA in water)). Separately, the combined fractions containing each product were basified with saturated NaHCO₃ (aq) and concentrated, followed by dilution with water and extraction with DCM (2×). The combined organic layers were concentrated to yield (283) (8.4 mg, 15% yield) and (283R) (8.8 mg, 16% yield). (283): ES-MS [M+1]⁺: 457.3, LCMS Retention time: 1.08 min, ¹H NMR (400 MHz, DMSO-d₆) δ 11.07 (br, 1H), 8.30 (br, 1H), 7.81 (br, 1H), 7.80 (s, 1H), 7.57-7.55 (m, 2H), 7.46-7.42 (m, 3H), 7.18 (d, J=2 Hz, 1H), 7.09 (dd, J=8.3, 2.1 Hz, 1H), 7.03 (d, J=8.3 Hz, 1H), 6.12 (s, 2H), 2.18 (s, 3H); (283R): ES-MS [M+1]⁺: 461.4.

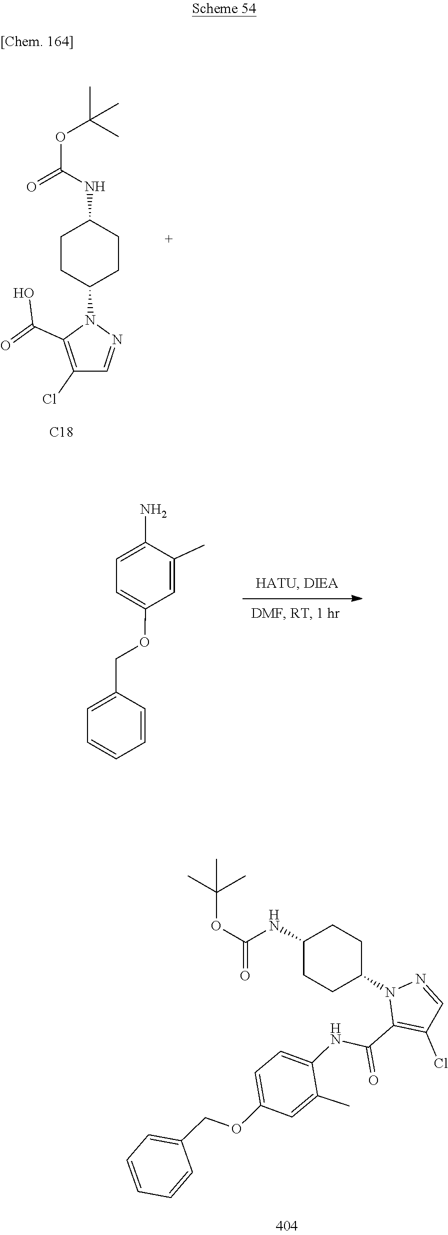 [SMILES: CC(C)(C)OC(=O)N[C@H]1CC[C@@H](n2ncc(Cl)c2C(=O)O)CC1 ; "C18" ; Cc1cc(OCc2ccccc2)ccc1N ; Cc1cc(OCc2ccccc2)ccc1NC(=O)c1c(Cl)cnn1[C@H]1CC[C@@H](NC(=O)OC(C)(C)C)CC1]

tert-butyl (cis-4-(5-((4-(benzyloxy)-2-methylphenyl)carbamoyl)-4-chloro-1H-pyrazol-1-yl)cyclo hexyl)carbamate (404): To a vial was added (C18) (20 mg, 0.06 mmol) and HATU (66.36 mg, 0.175 mmol). Then a solution of 4-benzyloxy-2-methylaniline (12.4 mg, 0.06 mmol) and DMF (0.29 mL) was added to the vial. Finally, N,N-diisopropylethylamine (0.05 mL, 0.29 mmol) was added to the solution, and the vial was capped and left at room temperature for 1 hour on benchtop. Afterwards, LCMS indicated product formation. Therefore, the sample was syringe-filtered, concentrated and re-suspended in DMSO. The sample was purified by reverse phase HPLC (gradient: 60-90% MeCN in water (0.1% TFA in water)). Fractions were neutralized with a saturated NaHCO₃ solution and then MeCN was evaporated. The product was extracted from the fractions with DCM, and then concentrated to yield (404) (20.9 mg, 66.6% yield). ES-MS [M-Boc+1]⁺: 439.2, ¹H NMR (400 MHz, CDCl₃) 58.06 (s, 1H), 7.71 (d, J=8.6 Hz, 1H), 7.54 (s, 1H), 7.44-7.31 (m, 5H), 6.89-6.85 (m, 2H), 5.25 (tt, J=10.9, 3.7 Hz, 1H), 5.07 (s, 2H), 4.87 (d, J=6.4 Hz, 1H), 3.86 (s, 1H), 2.32 (s, 3H), 2.10-2.01 (m, 2H), 1.94 (d, J=10.0 Hz, 4H), 1.75-1.68 (m, 2H), 1.45 (s, 9H).

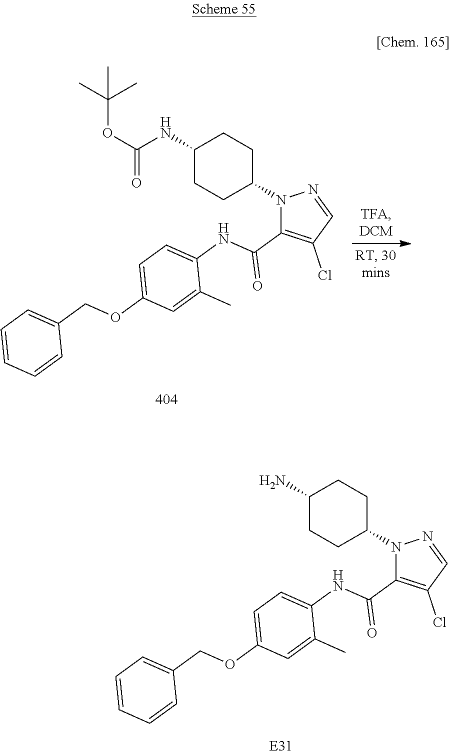 [SMILES: Cc1cc(OCc2ccccc2)ccc1NC(=O)c1c(Cl)cnn1[C@H]1CC[C@@H](NC(=O)OC(C)(C)C)CC1 ; Cc1cc(OCc2ccccc2)ccc1NC(=O)c1c(Cl)cnn1[C@H]1CC[C@@H](N)CC1]

1-(cis-4-aminocyclohexyl)-N-(4-(benzyloxy)-2-methylphenyl)-4-chloro-1H-pyrazole-5-carboxamide (E31): To a vial was added (404) (19.2 mg, 0.04 mmol), trifluoroacetic acid (0.05 mL) and DCM (0.200 mL). After 1 hr at rt, LCMS indicated desired product. The sample was neutralized with saturated NaHCO₃ solution, and the product was extracted in DCM. The organic extract was concentrated, re-suspended in DMSO, and then purified by reverse phase HPLC (gradient: 25-55% MeCN in water (0.1% TFA in water)). Fractions were neutralized with a saturated NaHCO₃ solution and then MeCN was evaporated. The product was extracted from the fractions with DCM, and then concentrated to yield (E31) (13 mg, 84% yield). ES-MS [M+1]⁺: 439.2, ¹H NMR (400 MHz, CDCl₃) δ 8.06 (s, 1H), 7.72 (d, J=8.5 Hz, 1H), 7.53 (s, 1H), 7.44-7.37 (m, 4H), 7.35-7.30 (m, 1H), 6.89-6.85 (m, 2H), 5.22 (tt, J=10.8, 4.0 Hz, 1H), 5.07 (s, 2H), 3.18 (p, J=3.5 Hz, 1H), 2.32 (s, 3H), 2.29-2.21 (m, 2H), 1.87-1.81 (m, 2H), 1.77-1.73 (m, 4H), 1.42 (br s, 2H).

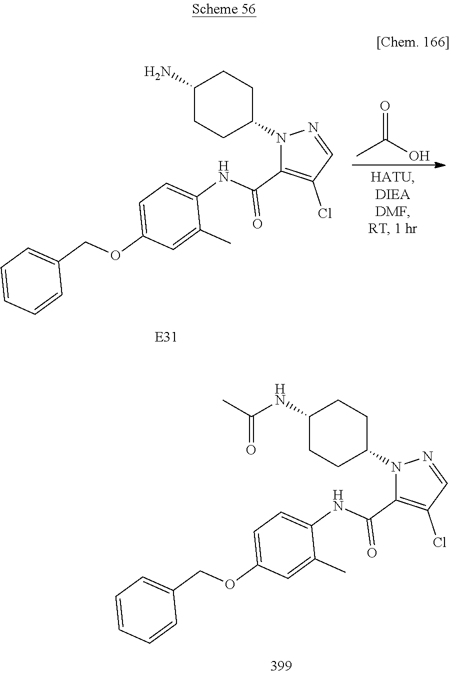

1-(cis-4-acetamidocyclohexyl)-N-(4-(benzyloxy)-2-methylphenyl)-4-chloro-1H-pyrazole-5-carboxamide (399): To a vial was added acetic acid (0.002 mL, 0.03 mmol) and HATU (17.15 mg, 05 mmol). Then a solution of (E31) (6.6 mg, 0.02 mmol) and DMF (0.40 mL) was added to the vial. Finally, N,N-diisopropylethylamine (0.01 mL, 0.08 mmol) was added to the solution. After 1 h at rt, LCMS indicated product formation. Therefore, the sample was syringe-filtered, concentrated and re-suspended in DMSO. The sample was purified by reverse phase HPLC (gradient: 50-80% MeCN in water (0.1% TFA in water)). Fractions were neutralized with a saturated NaHCO₃ solution and then MeCN was evaporated. The product was extracted from the fractions with DCM, and then concentrated to yield (399) (2 mg, 29% yield). ES-MS [M+1]⁺: 481.2, LCMS Retention time: 1.02 min, ¹H NMR (400 MHz, CDCl₃) δ 8.07 (s, 1H), 7.71 (d, J=8.6 Hz, 1H), 7.55 (s, 1H), 7.44-7.37 (m, 4H), 7.35-7.31 (m, 1H), 6.89-6.85 (m, 2H), 5.78 (d, J=7.4 Hz, 1H), 5.30-5.23 (m, 1H), 5.07 (s, 2H), 4.20-4.14 (m, 1H), 2.32 (s, 3H), 2.11-1.93 (m, 6H), 2.01 (s, 3H), 1.79-1.70 (m, 2H).

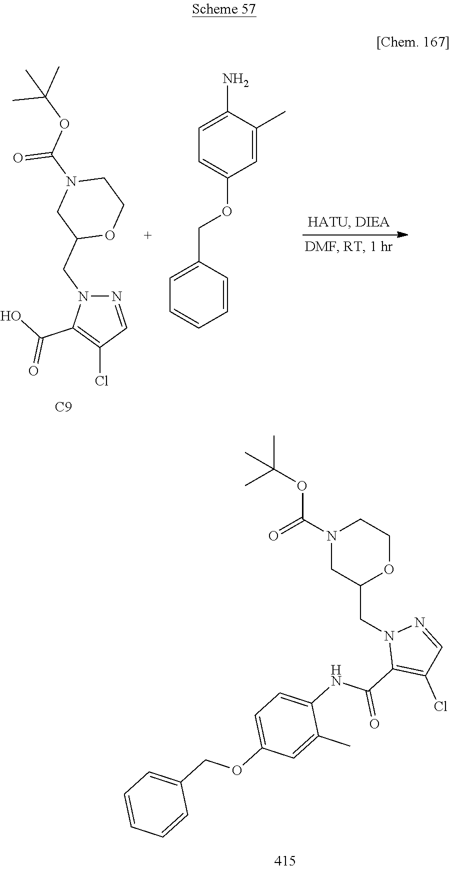

tert-butyl 2-((5-((4-(benzyloxy)-2-methylphenyl)carbamoyl)-4-chloro-1H-pyrazol-1-yl)methyl)morpholine-4-carboxylate (415): To a vial was added (C9) (22 mg, 0.06 mmol) and HATU (73 mg, 0.19 mmol). Then a solution of 4-benzyloxy-2-methylaniline (13.7 mg, 0.06 mmol) and DMF (0.35 mL) was added to the vial. Finally, N,N-diisopropylethylamine (0.06 mL, 0.32 mmol) was added to the solution, and the vial was capped and left at room temperature for 1 hour on benchtop. The sample was syringe-filtered, concentrated and re-suspended in DMSO. The sample was purified by reverse phase HPLC (gradient: 60-90% MeCN in water (0.1% TFA in water)). Fractions were neutralized with a saturated NaHCO₃ solution and then MeCN was evaporated. The product was extracted from the fractions with DCM, and then concentrated to yield (415) (18 mg, 53% yield). ES-MS [M-Boc+1]⁺: 441.2, LCMS Retention time: 1.20 min, ¹H NMR (400 MHz, CDCl₃) δ 8.11 (s, 1H), 7.70 (d, J=8.4 Hz, 1H), 7.55 (s, 1H), 7.44-7.31 (m, 5H), 6.89-6.85 (m, 2H), 5.01 (s, 2H), 4.76 (dd, J=13.5, 7.2 Hz, 1H), 4.67 (dd, J=13.7, 4.4 Hz, 1H) 3.93-3.78 (m, 4H), 3.44 (td, J=11.5, 2.7 Hz, 1H), 2.93 (t, J=11.1 Hz, 1H), 2.73 (t, J=11.2 Hz, 1H) 2.32 (s, 3H), 1.44 (s, 9H).

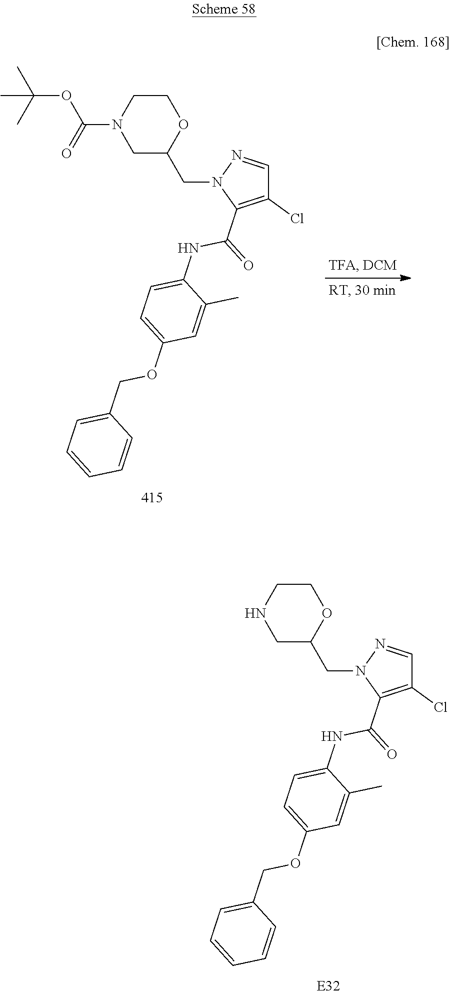 [SMILES: Cc1cc(OCc2ccccc2)ccc1NC(=O)c1c(Cl)cnn1CC1CN(C(=O)OC(C)(C)C)CCO1 ; Cc1cc(OCc2ccccc2)ccc1NC(=O)c1c(Cl)cnn1CC1CNCCO1]

N-(4-(benzyloxy)-2-methylphenyl)-4-chloro-1-(morpholin-2-ylmethyl)-1H-pyrazole-5-carboxamide (E32): To a vial was added (415) (17 mg, 0.03 mmol), trifluoroacetic acid (0.04 mL mg, 0.56 mmol) and DCM (0.15 mL). After 1 h at rt, LCMS indicated desired product. The sample was neutralized with saturated NaHCO₃ solution, and the product was extracted in DCM. The organic extract was concentrated to yield (E32) (15.0 mg, 110.9% yield) and carried forward as is. Higher yield due to trapped solvent. ES-MS [M+1]⁺: 441.2, ¹H NMR (400 MHz, CDCl₃) δ 8.14 (s, 1H), 7.69 (d, J=8.6 Hz, 1H), 7.54 (s, 1H), 7.44-7.37 (m, 4H), 7.35-7.31 (m, 1H), 6.89-6.85 (m, 2H), 5.07 (s, 2H), 4.66 (d, J=1.3 Hz, 1H), 4.65 (s, 1H), 3.93-3.84 (m, 2H), 3.51 (td, J=11.2, 2.8 Hz, 1H), 2.95 (dd, J=12.1, 1.1 Hz, 1H), 2.88-2.82 (m, 1H), 2.79-2.76 (m, 1H), 2.66-2.60 (m, 1H), 2.32 (s, 3H), 1.76 (br s, 1H).

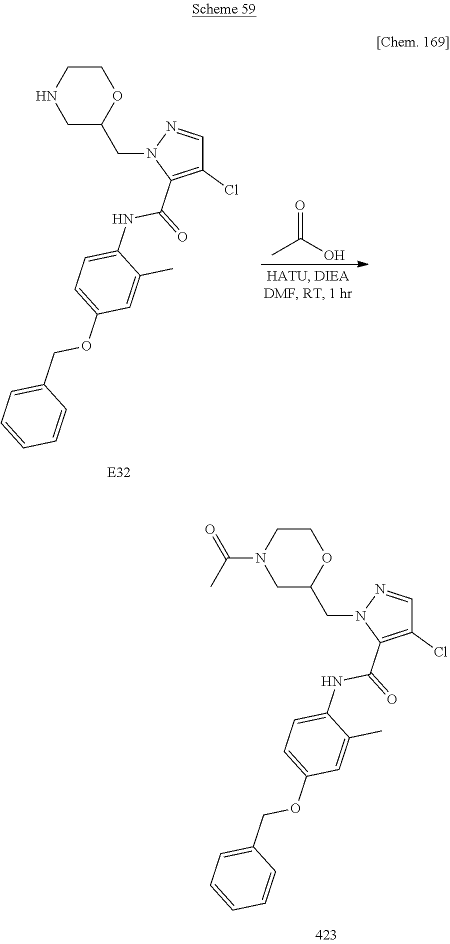

1-((4-acetylmorpholin-2-yl)methyl)-N-(4-(benzyloxy)-2-methylphenyl)-4-chloro-1H-pyrazole-5-carboxamide (423): To a vial was added acetic acid (0.002 mL, 0.03 mmol) and HATU (19.4 mg, 0.05 mmol). Then a solution of (E32) (7.5 mg, 0.02 mmol) and DMF (0.10 mL) was added to the vial. Finally, N,N-diisopropylethylamine (0.015 mL, 0.09 mmol) was added to the solution. After 1 h at rt, LCMS of the reaction indicated product formation. The sample was syringe-filtered, concentrated and re-suspended in DMSO. The sample was purified by reverse phase HPLC (gradient: 45-75% MeCN in water (0.1% TFA in water)). Fractions were neutralized with a saturated NaHCO₃ solution and then MeCN was evaporated. The product was extracted from the fractions with DCM, and then concentrated to yield (423) (6.2 mg, 76% yield). ES-MS [M-Boc+1]⁺: 483.3, LCMS Retention time: 0.96 min, ¹H NMR (400 MHz, CDCl₃) δ 8.12 (d, J=11.1 Hz, 1H), 7.70 (dd, J=16.4, 8.5 Hz, 1H), 7.56 (d, J=9.2 Hz, 1H), 7.44-7.37 (m, 4H), 7.35-7.31 (m, 1H), 6.90-6.85 (m, 2H), 5.07 (s, 2H), 4.84-7.68 (m, 2H), 4.43-4.31 (m, 1H), 3.94-3.85 (m, 2H), 3.75-3.40 (m, 2H), 3.30-3.02 (m, 1H), 2.96-2.60 (m, 1H), 2.32 (s, 3H), 2.07 (d, J=3.7 Hz, 3H).

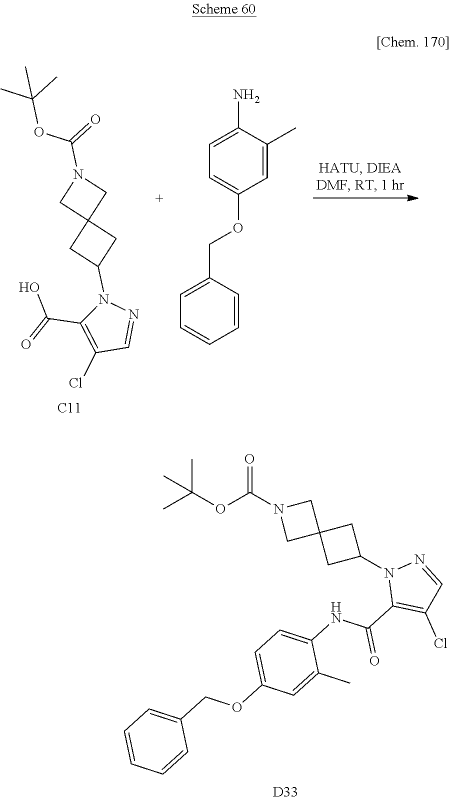 [SMILES: CC(C)(C)OC(=O)N1CC2(CC(n3ncc(Cl)c3C(=O)O)C2)C1 ; Cc1cc(OCc2ccccc2)ccc1N ; "C11" ; Cc1cc(OCc2ccccc2)ccc1NC(=O)c1c(Cl)cnn1C1CC2(C1)CN(C(=O)OC(C)(C)C)C2 ; "D33"]

tert-butyl 6-(5-((4-(benzyloxy)-2-methylphenyl)carbamoyl)-4-chloro-1H-pyrazol-1-yl)-2-azaspiro[3.3]heptane-2-carboxylate (D33): To a vial was added (C11) (19.5 mg, 0.06 mmol) and HATU (65.08 mg, 0.17 mmol). Then a solution of 4-benzyloxy-2-methylaniline (12.2 mg, 0.06 mmol) and DMF (0.30 mL) was added to the vial. Finally, N,N-diisopropylethylamine (0.05 mL, 0.29 mmol) was added to the solution. After 1 h at rt, LCMS of the reaction indicated product formation. The reaction mixture was syringe-filtered, concentrated and re-suspended in DMSO. The sample was purified by reverse phase HPLC (gradient: 60-90% MeCN in water (0.1% TFA in water)). Fractions were neutralized with a saturated NaHCO₃ solution and then MeCN was evaporated. The product was extracted from the fractions with DCM, and then concentrated to yield (D33) (19.7 mg, 64% yield). ES-MS [M+1]⁺: 537.2, ¹H NMR (400 MHz, CDCl₃) δ 8.06 (s, 1H), 7.70 (d, J=8.5 Hz, 1H), 7.55 (s, 1H), 7.44-7.37 (m, 4H), 7.35-7.30 (m, 1H), 6.89-6.85 (m, 2H), 5.65 (p, J=8.16 Hz, 1H), 5.07 (s, 2H), 4.01 (s, 2H), 3.94 (s, 2H), 2.85-2.79 (m, 2H), 2.73-2.68 (m, 2H), 2.31 (s, 3H), 1.43 (s, 9H).

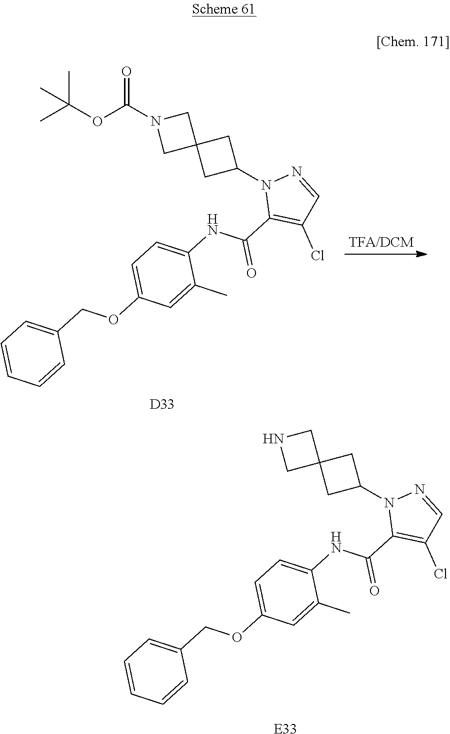 [SMILES: Cc1cc(OCc2ccccc2)ccc1NC(=O)c1c(Cl)cnn1C1CC2(C1)CN(C(=O)OC(C)(C)C)C2 ; Cc1cc(OCc2ccccc2)ccc1NC(=O)c1c(Cl)cnn1C1CC2(CNC2)C1 ; "D33" ; "E33"]

N-(4-(benzyloxy)-2-methylphenyl)-4-chloro-1-(2-azaspiro[3.3]heptan-6-yl)-1H-pyrazole-5-carboxamide (E33): To a vial was added (D33) (44.7 mg, 0.08 mmol), trifluoroacetic acid (0.117 mL mg, 1.53 mmol) and DCM (0.42 mL). After 1.5 h at rt, LCMS of the reaction indicated boc group removal. Therefore, the sample was neutralized with saturated NaHCO₃ solution, and the product was extracted in DCM. The organic extract was concentrated, re-suspended in DMSO, and then purified by reverse phase HPLC (gradient: 25-65% MeCN in water (0.1% TFA in water)). Fractions were neutralized with a saturated NaHCO₃ solution and then MeCN was evaporated. The product was extracted from the fractions with DCM, and then concentrated to yield (E33) (24.3 mg, 67% yield). ES-MS [M+1]⁺: 437.4, ¹H NMR (400 MHz, CDCl₃) δ 8.06 (m, 1H), 7.69 (d, J=8.5 Hz, 1H), 7.54 (m, 1H), 7.44-7.36 (m, 4H), 7.34-7.30 (m, 1H), 6.88-6.84 (m, 2H), 5.61 (p, J=8.2 Hz, 1H), 5.06 (s, 2H), 3.71 (d, J=28.9 Hz, 3H), 3.28 (d, J=26.9 Hz, 1H), 2.79-2.61 (m, 4H), 2.50 (br s, 1H), 2.31 (s, 3H).

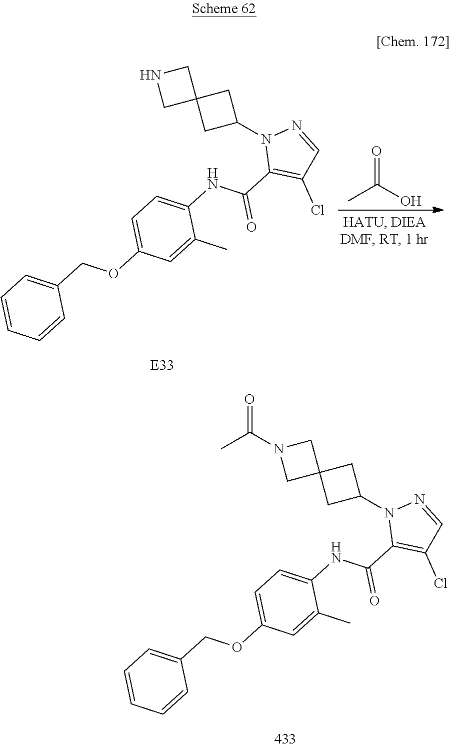

1-(2-acetyl-2-azaspiro[3.3]heptan-6-yl)-N-(4-(benzyloxy)-2-methylphenyl)-4-chloro-1H-pyrazole-5-carboxamide (433): To a vial was added acetic acid (0.002 mL, 0.04 mmol) and HATU (21.1 mg, 0.06 mmol). Then a solution of (E33) (8.1 mg, 0.02 mmol) and DMF (0.452 mL) was added to the vial. N,N-diisopropylethylamine (0.02 mL, 0.09 mmol) was added to the solution. After 1 h at rt, the sample was filtered, concentrated and dissolved in DMSO. The sample was purified by reverse phase HPLC (gradient: 50-80% MeCN in water (0.1% TFA in water)). Fractions were neutralized with a saturated NaHCO₃ solution and then MeCN was evaporated. The product was extracted from the fractions with DCM, and then concentrated to yield (433) (6.0 mg, 68% yield). ES-MS [M+1]⁺: 479.2, LCMS Retention time: 1.04 min, ¹H NMR (400 MHz, CDCl₃) δ 8.07 (d, J=11.4 Hz, 1H), 7.71-7.67 (m, 1H), 7.56 (s, 1H), 7.44-7.37 (m, 4H), 7.35-7.31 (m, 1H), 6.89-6.85 (m, 2H), 5.74-5.65 (m, 1H), 5.07 (s, 2H), 4.20 (s, 1H), 4.14 (s, 1H), 4.07 (s, 1H), 4.02 (s, 1H), 2.90-2.83 (m, 2H), 2.78-2.70 (m, 2H), 2.31 (d, J=1.4 Hz, 3H), 1.86 (d, J=2.9 Hz, 3H).

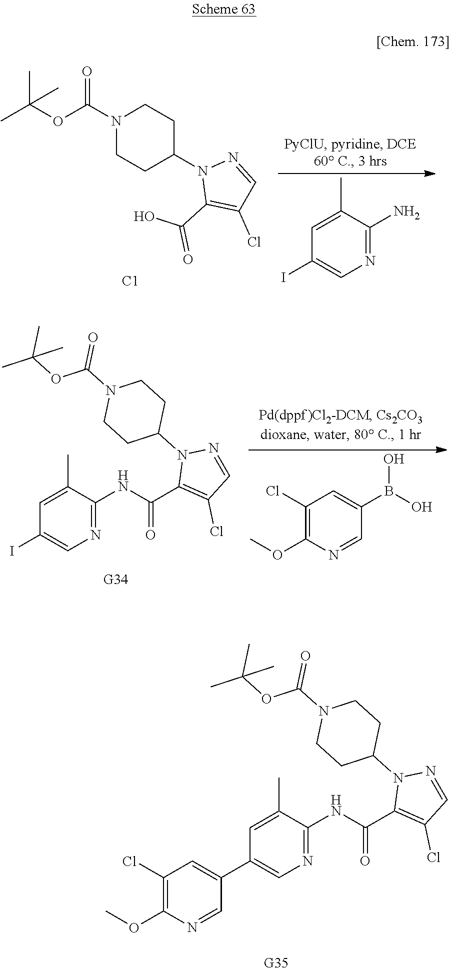

tert-butyl 4-(4-chloro-5-((5′-chloro-6′-methoxy-5-methyl-[3,3′-bipyridin]-6-yl)carbamoyl)-1H-pyrazol-1-yl)piperidine-1-carboxylate (G35): To a solution of (C1) (1000 mg, 3.03 mmol), 5-iodo-2-amino-3-picoline (922 mg, 3.94 mmol), and PyClU (2017 mg, 6.06 mmol) in DCE (10 mL) was added pyridine (2445 mg, 30.9 mmol). After 3 hours at 60° C., the solution was diluted with water (500 mL). The reaction mixture was extracted with EtOAc 1×500 mL. The organic layer was concentrated and purified by normal-phase flash chromatography (gradient: 0-50% EtOAc in hexanes). The fractions that contain product were concentrated to yield (G34) (1203 mg, 73% yield). ES-MS [M+1]⁺: 546.2.

A solution of (G34) (50 mg, 0.09 mmol), 3-chloro-2-methoxypyridine-5-boronic acid (34 mg, 0.18 mmol), Pd(dppf)Cl₂-DCM adduct (7.5 mg, 0.009 mmol), and Cs₂CO₃ (66 mg, 0.20 mmol) in dioxane (0.5 mL) and water (0.1 mL) was heated to 80° C. for 1 hour. The solution was removed from heat and partially concentrated. The solution was purified by reverse phase HPLC (gradient: 35-95% MeCN in water (water modified with 0.1% TFA)). The fractions were concentrated to yield (G35) (38 mg, 62% yield). ES-MS [M+1]⁺: 561.4.

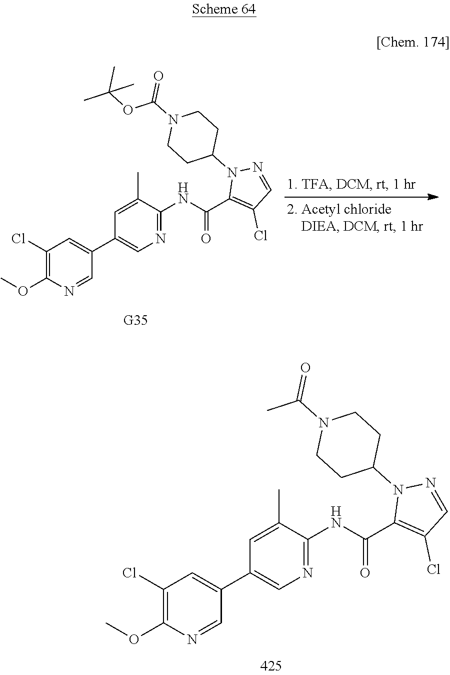 [SMILES: COc1ncc(-c2cnc(NC(=O)c3c(Cl)cnn3C3CCN(C(=O)OC(C)(C)C)CC3)c(C)c2)cc1Cl ; COc1ncc(-c2cnc(NC(=O)c3c(Cl)cnn3C3CCN(C(C)=O)CC3)c(C)c2)cc1Cl]

1-(1-acetylpiperidin-4-yl)-4-chloro-N-(5′-chloro-6′-methoxy-5-methyl-[3,3′-bipyridin]-6-yl)-1H-pyrazole-5-carboxamide (425): To a solution of (G35) (38 mg, 0.07 mmol), in DCM (1 mL), was added TFA (0.25 mL). After 1 hour at room temperature, the solution was concentrated. To the residue was added DCM (1.2 mL), DIEA (26 mg, 0.20 mmol), and acetyl chloride (6.4 mg, 0.08 mmol). After 1 hour at room temperature, the solution was concentrated. The product was purified by reverse phase HPLC (gradient: 25-75% MeCN in water (water modified with 0.1% TFA)). The fractions that contain isolated product were combined and diluted with water and NaHCO₃ solution. The dilution was extracted with EtOAc 2×5 mL. The combined organic layers were washed with water 2×10 mL and concentrated to yield (425) (18 mg, 52% yield). ES-MS [M+1]⁺: 503.2, LCMS Retention time: 0.96 min, ¹H NMR (400 MHz, CDCl₃) δ 8.68-8.54 (br m, 2H), 8.27 (s, 1H), 7.84 (s, 1H), 7.80 (s, 1H), 7.58 (s, 1H), 5.45 (br s, 1H), 4.75 (d, J=12.0 Hz, 1H), 4.09 (s, 3H), 3.94 (d, J=11.5 Hz), 3.21 (br s, 1H), 2.70 (br s, 1H), 2.46 (s, 3H), 2.14-2.08 (m, 4H), 2.12 (s, 3H).

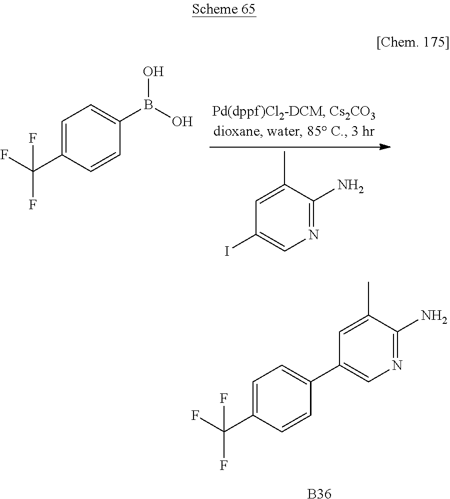

3-methyl-5-(4-(trifluoromethyl)phenyl)pyridin-2-amine (B36): A mixture of 5-iodo-2-amino-3-picoline (100 mg, 0.43 mmol), 4-(trifluoromethyl)phenylboronic acid (162 mg, 0.86 mmol), Pd(dppf)Cl₂-DCM adduct (35 mg, 0.04 mmol), and Cs₂CO₃ (308 mg, 0.94 mmol) in dioxane (4 mL) and water (0.4 mL) was heated to 85° C. for 3 hours. The solution was removed from heat and diluted with water. The reaction mixture was extracted 2×25 mL with EtOAc, and washed with water 2×50 mL. The organic layers were concentrated and purified by normal-phase flash chromatography (gradient: 0-10% MeOH in DCM). Fractions that contain product were combined and concentrated to yield (B36) (108 mg, 99% yield). ES-MS [M+1]⁺: 253.4.

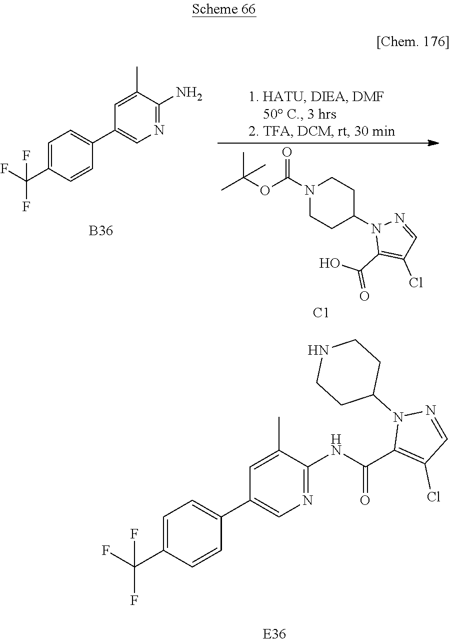 [SMILES: "B36" ; Cc1cc(-c2ccc(C(F)(F)F)cc2)cnc1N ; CC(C)(C)OC(=O)N1CCC(n2ncc(Cl)c2C(=O)O)CC1 ; "C1" ; Cc1cc(-c2ccc(C(F)(F)F)cc2)cnc1NC(=O)c1c(Cl)cnn1C1CCNCC1]

4-chloro-N-(3-methyl-5-(4-(trifluoromethyl)phenyl)pyridin-2-yl)-1-(piperidin-4-yl)-1H-pyrazole-5-carboxamide (E36): To a solution of (C1) (62 mg, 0.19 mmol), (B36) (47 mg, 0.19 mmol), and HATU (142 mg, 0.37 mmol) in DMF (1 mL) was added DIEA (142 mg, 0.47 mmol). After 3 hours at 50° C., the solution was diluted with water and extracted with EtOAc 2×10 mL. The organic layers were combined and washed with water 1×10 mL. The organic layer was concentrated and purified by reverse phase HPLC (gradient: 35-95% MeCN in water (water modified with 0.1% TFA in water)). The fractions that contain isolated product were combined and concentrated in vacuo. The resulting residue was suspended in DCM (10 mL) and TFA (1 mL). After 30 minutes at room temperature, the solution was concentrated to yield (E36) as a TFA salt (31 mg, 29% yield).

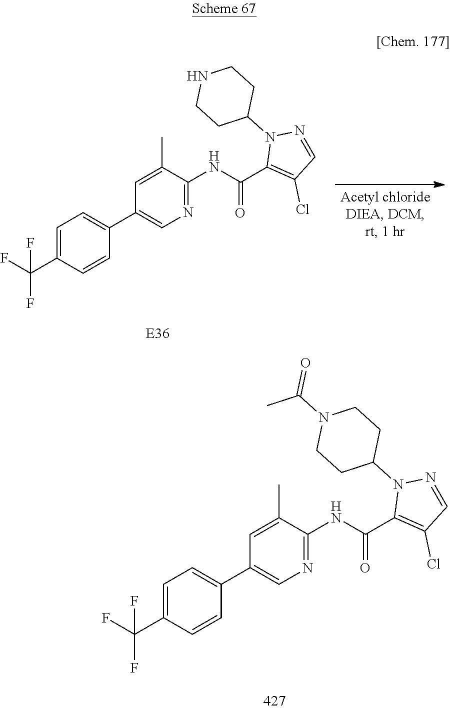 [SMILES: Cc1cc(-c2ccc(C(F)(F)F)cc2)cnc1NC(=O)c1c(Cl)cnn1C1CCNCC1 ; CC(=O)N1CCC(n2ncc(Cl)c2C(=O)Nc2ncc(-c3ccc(C(F)(F)F)cc3)cc2C)CC1]

1-(1-acetylpiperidin-4-yl)-4-chloro-N-(3-methyl-5-(4-(trifluoromethyl)phenyl)pyridin-2-yl)-1H-pyrazole-5-carboxamide (Compound 427): To a solution of (E36) (31 mg, 0.05 mmol) and DIEA (21 mg, 0.16 mmol) in DCM was added acetyl chloride (5 mg, 0.06 mmol). After 1 hour at room temperature the solution was partially concentrated and purified by reverse phase HPLC (gradient: 30-90% MeCN in water (water is modified with 0.1% TFA)). The fractions that contain product were combined and diluted with water and NaHCO₃ solution. The dilution was extracted with EtOAc 2×5 mL. The extractions were combined and washed with water 2×10 mL and concentrated yield (427) (12 mg, 43% yield). ES-MS [M+1]⁺: 506.3, LCMS Retention time: 1.05 min.

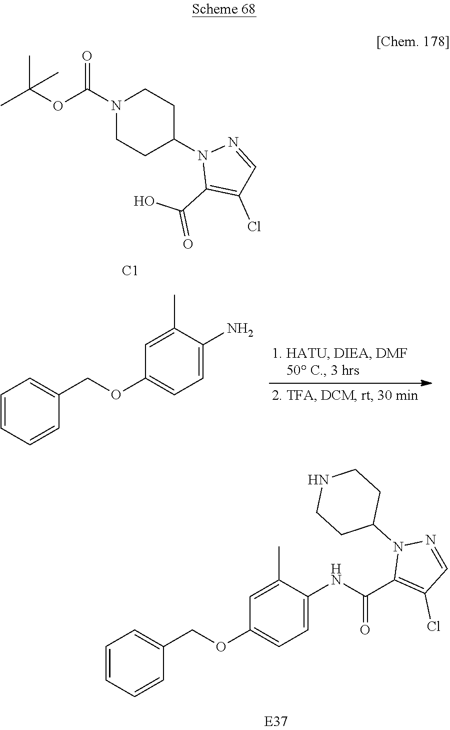 [SMILES: CC(C)(C)OC(=O)N1CCC(n2ncc(Cl)c2C(=O)O)CC1 ; "C1" ; Cc1cc(OCc2ccccc2)ccc1N ; Cc1cc(OCc2ccccc2)ccc1NC(=O)c1c(Cl)cnn1C1CCNCC1]

N-(4-(benzyloxy)-2-methylphenyl)-4-chloro-1-(piperidin-4-yl)-1H-pyrazole-5-carboxamide (E37): To a solution of (C1) (30 mg, 0.09 mmol), 4-benxyloxy-2-methylaniline (19 mg, 0.09 mmol), and HATU (69 mg, 0.18 mmol) in DMF (0.5 mL) was added DIEA (29 mg, 0.23 mmol). After 3 hours at 50° C., the solution was diluted with water and extracted with EtOAc 2×10 mL. The combined organic layers were washed with water (IxI0 mL), concentrated and purified by reverse phase HPLC (gradient: 35-95% MeCN in water (water modified with 0.1% TFA in water)). The fractions that contain isolated product were combined and concentrated in vacuo. The resulting residue was suspended in DCM (5 mL) and TFA (1 mL). After 30 minutes at room temperature, the solution was concentrated to yield (E37) as a TFA salt (12 mg, 24% yield).

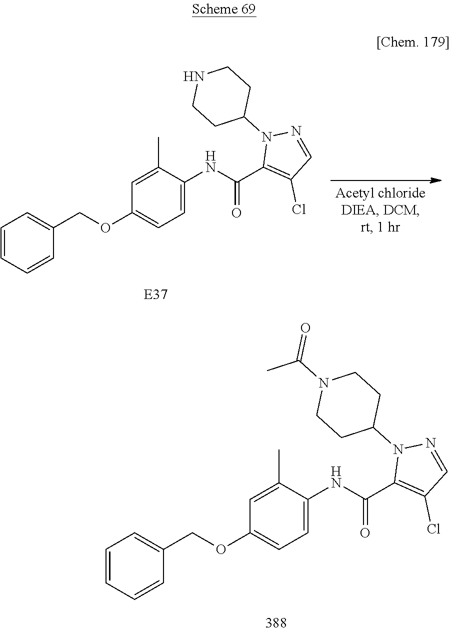 [SMILES: Cc1cc(OCc2ccccc2)ccc1NC(=O)c1c(Cl)cnn1C1CCNCC1 ; CC(=O)N1CCC(n2ncc(Cl)c2C(=O)Nc2ccc(OCc3ccccc3)cc2C)CC1]

1-(1-acetylpiperidin-4-yl)-N-(4-(benzyloxy)-2-methylphenyl)-4-chloro-1H-pyrazole-5-carboxamide (388): To a solution of (E37) (11 mg, 0.02 mmol), and DIEA (8 mg, 0.06 mmol) in DCM (0.5 mL) was added acetyl chloride (2 mg, 0.03 mmol). After 1 hour at room temperature, the solution was concentrated and the product was purified by reverse phase HPLC (gradient: 35-95% MeCN in water (water is modified with 0.5 mL of NH₄OH per 1 L of water)). The fractions that contain product were concentrated to yield (388) (5.2 mg, 55% yield). ES-MS [M+1]⁺: 466.0, LCMS Retention time: 1.07 min, ¹H NMR (400 MHz, CDCl₃) δ 8.13 (s, 1H), 7.70 (d, J=8.5 Hz, 1H), 7.54 (s, 1H), 7.44-7.31 (m, 5H), 6.90-6.86 (m, 2H), 5.52-5.45 (m, 1H), 5.07 (s, 2H), 4.74 (br s, 1H), 3.93 (br s, 1H), 3.22 (br s, 1H), 2.71 (br s, 1H), 2.32 (s, 3H), 2.17-2.03 (m, 4H), 2.13 (s, 3H).

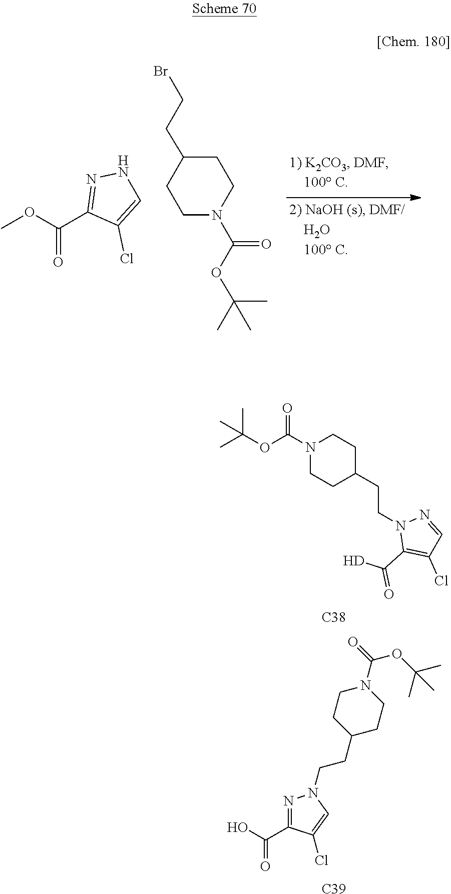

1-(2-(1-(tert-butoxycarbonyl)piperidin-4-yl)ethyl)-4-chloro-1H-pyrazole-5-carboxyli c acid (C38) and 1-(2-(1-(tert-butoxycarbonyl)piperidin-4-yl)ethyl)-4-chloro-1H-pyrazole-3-carboxylic acid (C39): Compound C38 and C39 are prepared in a similar manner as Compound C16 and C17. C38: (124 mg, 34% yield). ES-MS [M+Na]⁺: 380.3, ¹H NMR (400 MHz, DMSO) δ 7.69 (s, 1H), 4.53-4.45 (m, 2H), 3.88 (d, J=13.1 Hz, 2H), 2.76-2.55 (m, 2H), 1.71-1.60 (m, 4H), 1.38 (s, 9H), 1.37-1.27 (m, 1H), 0.99 (dddd, J=12.28, 12.25, 12.25, 4.3 Hz, 2H). C39: (129 mg, 35% yield). ES-MS [M+Na]⁺: 380.4, 1 H NMR (400 MHz, DMSO) δ 8.14 (s, 1H), 4.16 (t, J=7.3 Hz, 2H), 3.89 (d, J=13.1 Hz, 2H), 2.78-2.54 (m, 2H), 1.73 (q, J=7.1 Hz, 2H), 1.64 (d, J=12.9 Hz, 2H), 1.38 (s, 9H), 1.36-1.27 (m, 1H), 1.00 (dddd, J=12.28, 12.08, 12.08, 4.3 Hz, 2H).

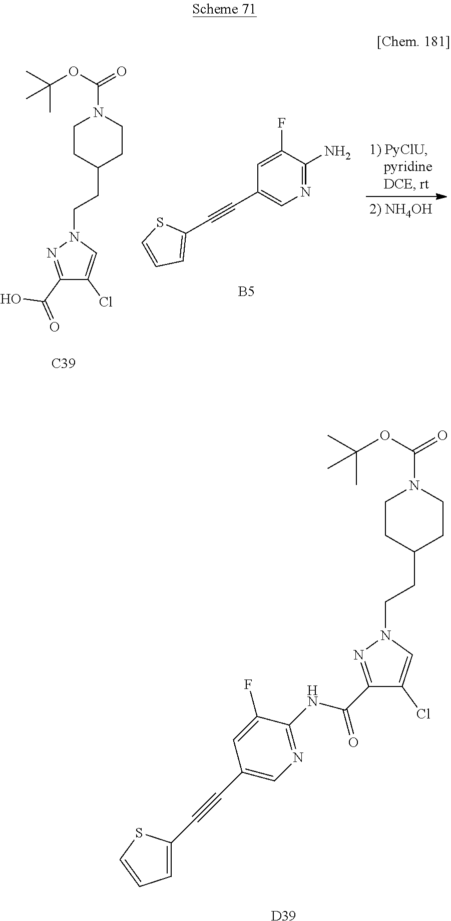

tert-butyl 4-(2-(4-chloro-3-((3-fluoro-5-(thiophen-2-ylethynyl)pyridin-2-yl)carbamoyl)-1H-pyrazol-1-yl)ethyl)piperidine-1-carboxylate (D39): Compound D39 is prepared in a similar manner as Compound 328 to yield the title compound D39 (59 mg, 79% yield). ES-MS [M+1]⁺: 558.2 ¹H NMR (400 MHz, DMSO) δ 10.46 (s, 1H), 8.49-8.47 (m, 1H), 8.22 (s, 1H), 8.08 (dd, J=10.5, 1.8 Hz, 1H), 7.75 (dd, J=5.1, 1.2 Hz, 1H), 7.50 (dd, J=3.6, 1.2 Hz, 1H), 7.17 (dd, J=5.1, 3.6 Hz, 1H), 4.21 (t, J=7.1 Hz, 3H), 3.90 (d, J=13.0 Hz, 2H), 2.77-2.56 (m, 2H), 1.82-1.75 (m, 2H), 1.71-1.63 (m, 2H), 1.38 (s, 9H), 1.02 (dddd, J=12.30, 12.25, 12.25, 4.3 Hz, 2H).

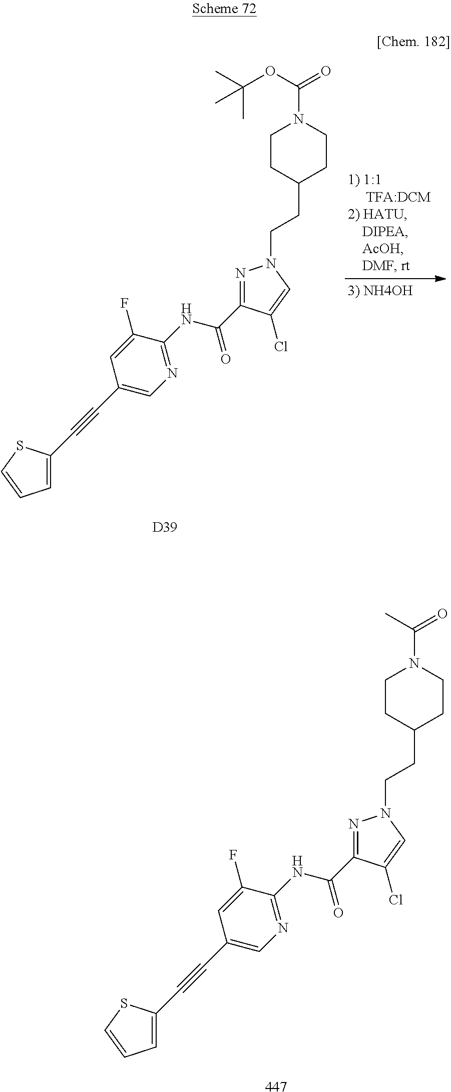 [SMILES: CC(C)(C)OC(=O)N1CCC(CCn2cc(Cl)c(C(=O)Nc3ncc(C#Cc4cccs4)cc3F)n2)CC1 ; CC(=O)N1CCC(CCn2cc(Cl)c(C(=O)Nc3ncc(C#Cc4cccs4)cc3F)n2)CC1 ; "D39" ; "447"]

1-(2-(1-acetylpiperidin-4-yl)ethyl)-4-chloro-N-(3-fluoro-5-(thiophen-2-ylethynyl)pyridin-2-yl)-1H-pyrazole-3-carboxamide (447): Compound 447 is prepared in a similar manner as Compound 5 to yield the title compound 447 (7 mg, 72% yield). ES-MS [M+1]⁺: 500.0, LCMS Retention time: 0.98 min, ¹H NMR (400 MHz, DMSO) δ 10.47 (s, 1H), 8.49 (dd, J=1.9, 0.7 Hz, 1H), 8.23 (s, 1H), 8.08 (dd, J=10.4, 1.8 Hz, 1H), 7.75 (dd, J=5.2, 1.2 Hz, 1H), 7.50 (dd, J=3.6, 1.2 Hz, 1H), 7.17 (dd, J=5.1, 3.6 Hz, 1H), 4.37-4.29 (m, 1H), 4.22 (t, J=7.2 Hz, 2H), 3.80-3.72 (m, 1H), 3.02-2.92 (m, 2H), 1.97 (s, 3H), 1.80 (dddd, J=9.1, 9.1, 6.7, 6.7 Hz, 2H), 1.76-1.65 (m, 1H), 1.50-1.38 (m, 1H), 1.10 (dddd, J=12.2, 12.2, 12.2, 4.1 Hz, 1H), 0.97 (dddd, J=12.3, 12.2, 12.2, 4.2 Hz, 1H).

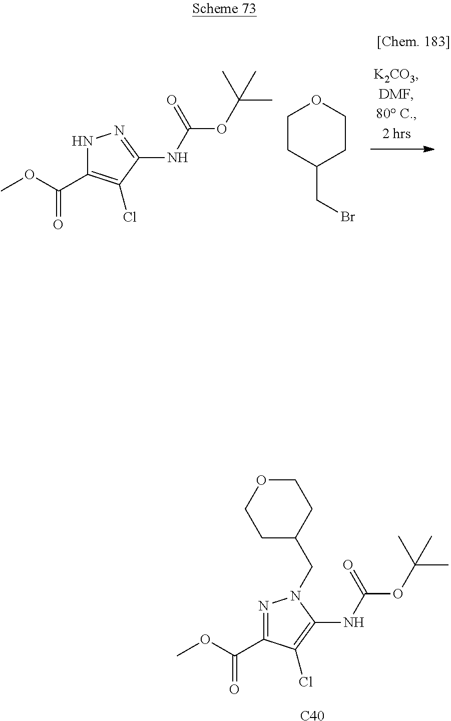

methyl 5-((tert-butoxycarbonyl)amino)-4-chloro-1-((tetrahydro-2H-pyran-4-yl)methyl)-1H-pyrazole-3-carboxylate (C40): A solution of methyl 3-(tert-butoxycarbonylamino)-4-chloro-1H-pyrazole-5-carboxylate (100 mg, 0.36 mmol), 4-(bromomethyl)oxan (97 mg, 0.54 mmol) and potassium carbonate (153 mg, 1.09 mmol) in DMF (2 mL) was heated to 80° C. for 2 hours. The reaction mixture was purified by reverse phase HPLC (gradient: 20-80% MeCN in water (0.1% TFA in water)). Fractions that contained the isolated intermediate were concentrated to yield (C40) (32 mg, 24% yield); ¹H NMR (400 MHz, d6-DMSO) δ 9.31 (br s, 1H), 3.94 (t, J=7.4 Hz, 2H), 3.85-3.79 (m, 2H), 3.81 (s, 3H), 3.22 (m, 2H), 2.05 (m, 1H), 1.43-1.40 (m, 2H), 1.38 (s, 3H), 1.32-1.19 (m, 2H).

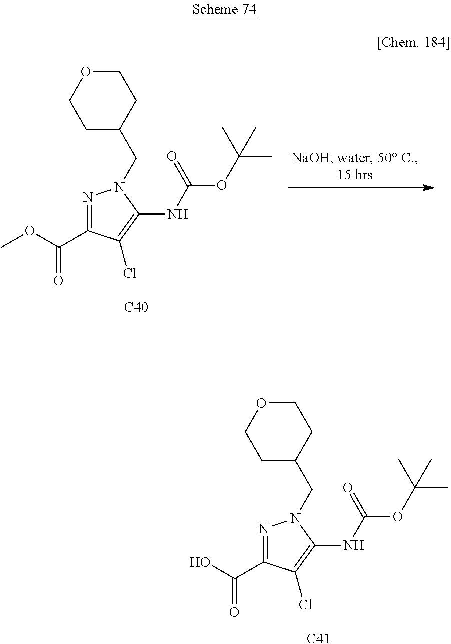 [SMILES: COC(=O)c1nn(CC2CCOCC2)c(NC(=O)OC(C)(C)C)c1Cl ; "C40" ; CC(C)(C)OC(=O)Nc1c(Cl)c(C(=O)O)nn1CC1CCOCC1 ; "C41"]

5-((tert-butoxycarbonyl)amino)-4-chloro-1-((tetrahydro-2H-pyran-4-yl)methyl)-1H-pyrazole-3-carboxylic acid (C41): To a solution of water (0.582 mL) and (C40) (55 mg, 0.15 mmol) was added 2 mol/L NaOH_{(aq)} (0.60 mL, 1.2 mmol). After 15 hours at 50° C., the solution was neutralized by dropwise addition of 6M HCl_{(aq)} solution. The mixture was concentrated and the resulting solids were sonicated with EtOH and the slurry was filtered to yield (C41) (18 mg, 58% yield); ES-MS [M+H]⁺: 360.3.

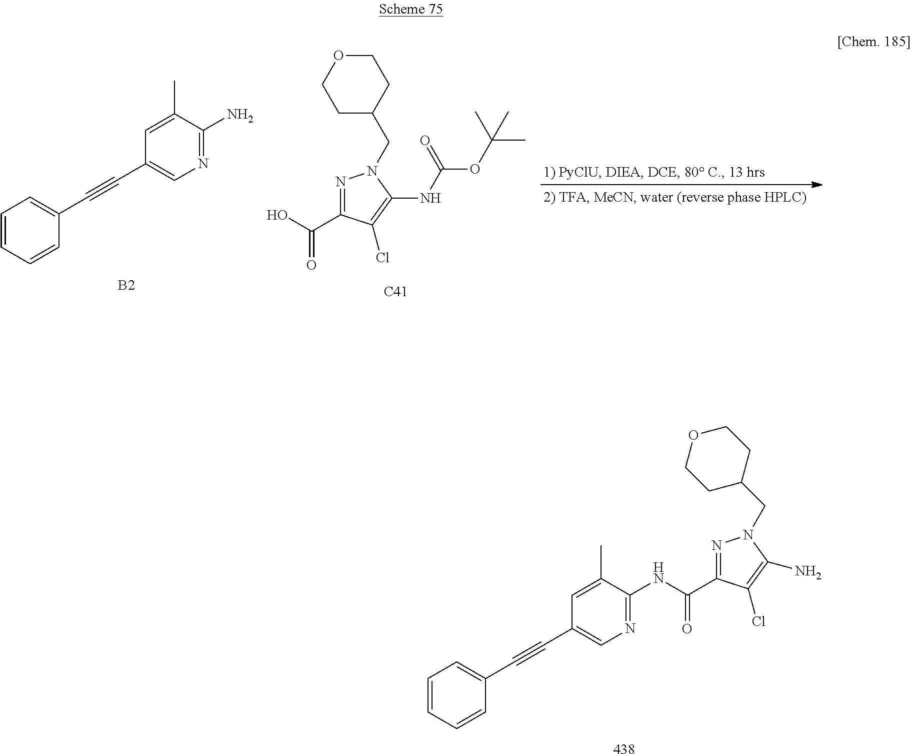 [SMILES: Cc1cc(C#Cc2ccccc2)cnc1N ; CC(C)(C)OC(=O)Nc1c(Cl)c(C(=O)O)nn1CC1CCOCC1 ; "B2" ; "C41" ; Cc1cc(C#Cc2ccccc2)cnc1NC(=O)c1nn(CC2CCOCC2)c(N)c1Cl ; "438"]

5-amino-4-chloro-N-(3-methyl-5-(phenylethynyl)pyridin-2-yl)-1-((tetrahydro-2H-pyran-4-yl)methyl)-1H-pyrazole-3-carboxamide (438): A solution of (C41) (15 mg, 0.04 mmol), (B2) (13 mg, 0.06 mmol), PyClU (28 mg, 0.08 mmol) and DIEA (0.04 mL, 0.21 mmol) in DCE (0.5 mL) was heated to 80° C. After 13 h, the solution was purified by reverse phase HPLC (gradient: 45-95% MeCN in water (0.1% TFA in water)). Fractions that contained isolated product were combined and concentrated to yield the TFA salt of (438). The residue was diluted in MeOH. To the dilution was added MP-carbonate. The solution was filtered and concentrated to yield (438) (8.2 mg, 44% yield), ES-MS [M+H]⁺: 450.2, LCMS Retention time: 1.03 min, ¹H NMR (400 MHz, CDCl₃) δ 8.46 (d, J=1.8 Hz, 1H), 7.92 (d, J=1.1 Hz, 1H), 7.56-7.53 (m, 2H), 7.40-7.37 (m, 3H), 4.01-3.98 (m, 2H), 3.91 (d, J=7.3 Hz, 2H), 3.41 (ddd, J=11.7, 11.7, 1.7 Hz, 2H), 2.42 (s, 3H), 2.30-2.22 (m, 1H), 1.61-1.57 (m, 2H), 1.46-1.36 (m, 2H).

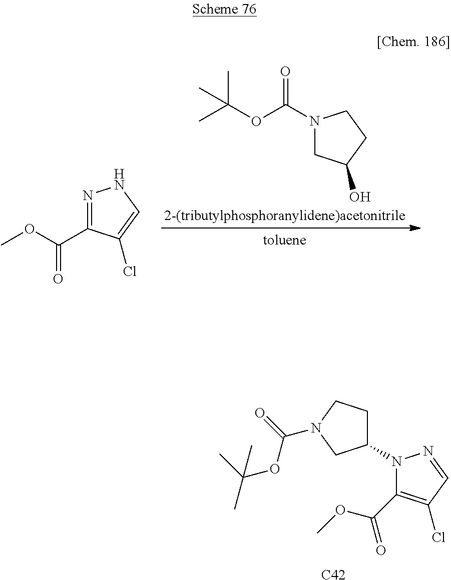

Methyl (S)-1-(1-(tert-butoxycarbonyl)pyrrolidin-3-yl)-4-chloro-1H-pyrazole-5-carboxylate (C42): To a solution of methyl 4-chloro-1H-pyrazole-5-carboxylate (1 g, 6.23 mmol) and (R)-tert-butyl 3-hydroxypyrrolidine-1-carboxylate (1283 mg, 6.85 mmol) in Toluene (15 mL) was added 2-(tributylphosphoranylidene)acetonitrile (2255 mg, 9.34 mmol). The reaction mixture was stirred at 120° C. for 16 hrs. The reaction mixture was concentrated in vacuum. The residue was purified by flash column (0%-25% EA in PE) to afford (C42) (1.3 g, 3.94 mmol, 63% yield) as a yellow oil.*¹ ESI-MS [M-tBu+2H]⁺: 274, ¹H NMR (400 MHz, CDCl₃): δ 7.48 (m, 1H), 5.71 (m, 1H), 3.95 (s, 3H), 3.79 (m, 1H), 3.69-3.61 (m, 2H), 3.50 (m, 1H), 2.45 (m, 1H), 2.30 (m, 1H), 1.45 (s, 9H). *1: The data were measured by Reversed-phase LCMS method (2) as described above.

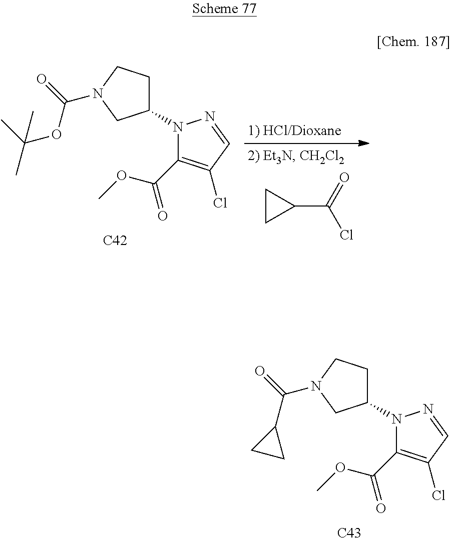 [SMILES: COC(=O)c1c(Cl)cnn1[C@H]1CCN(C(=O)OC(C)(C)C)C1 ; O=C(Cl)C1CC1 ; "C42" ; COC(=O)c1c(Cl)cnn1[C@H]1CCN(C(=O)C2CC2)C1 ; "C43"]

Methyl (S)-4-chloro-1-(1-(cyclopropanecarbonyl)pyrrolidin-3-yl)-1H-pyrazole-5-carboxylate (C43): A solution of (C42) (1.3 g, 3.94 mmol) in HCl/Dioxane (5 mL, 20.0 mmol) was stirred at 20° C. for 2 h. After compound C42 was consumed on TLC, the reaction mixture was concentrated to yield methyl 4-chloro-2-[(3S)-pyrrolidin-3-yl]pyrazole-3-carboxylate hydrochloride (1 g, 3.76 mmol) as a white solid which was subsequently used without further purification. To a solution of methyl 4-chloro-2-[(3S)-pyrrolidin-3-yl]pyrazole-3-carboxylate hydrochloride (1 g, 3.76 mmol) in DCM (5 mL) was added TEA (2.61 mL, 18.79 mmol) and cyclopropanecarbonyl chloride (0.41 mL, 4.51 mmol) at 0° C. The resulting mixture was stirred at 0° C. for 0.5 hours to give a yellow solution. After confirming a new spot on TLC (Rf: 0.4, PE/EtOAc=1/1), the reaction mixture was concentrated, poured into water (10 mL), and extracted with EtOAc (10 mL×4). The combined organic layers were washed with brine (10 mL×2) and dried over Na₂SO₄. The filtrate was concentrated under reduced pressure. The residue was purified by flash column (PE to 50% EtOAc in PE) to afford (C43) (1 g, 3.36 mmol, 89% yield) as a white solid. ¹H NMR (400 MHz, CDCl₃): δ 7.51-7.48 (m, 1H), 5.86-5.74 (m, 1H), 4.18-3.76 (m, 8H), 2.63-2.34 (m, 2H), 1.04-0.99 (m, 2H), 0.80-0.76 (m, 2H).

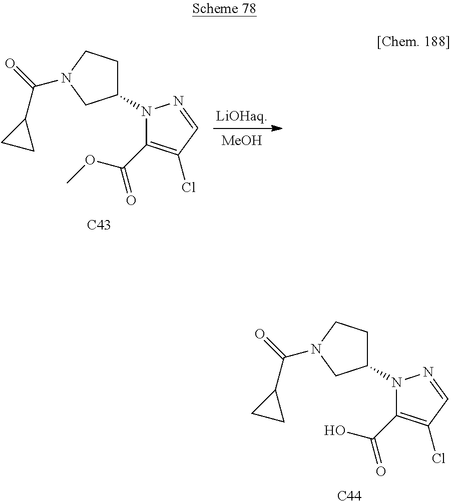 [SMILES: "MeOH" ; COC(=O)c1c(Cl)cnn1[C@H]1CCN(C(=O)C2CC2)C1 ; "C43" ; O=C(O)c1c(Cl)cnn1[C@H]1CCN(C(=O)C2CC2)C1 ; "C44"]

(S)-4-chloro-1-(1-(cyclopropanecarbonyl)pyrrolidin-3-yl)-1H-pyrazole-5-carboxylic acid (C44): To a solution of (C43) (1 g, 3.36 mmol) in Methanol (5 mL) and Water (5 mL) was added LiOH. H₂O (282 mg, 6.72 mmol). The mixture was stirred at 20° C. for 4 h. After compound C43 was consumed on TLC, the reaction mixture was poured into water (50 mL) and acidified with 1 mol/L HCl-aq to pH=2. The aqueous mixture was extracted with DCM (50 mL×4). The combined organic layers were washed with sat. NaHCO₃-aq. (50 mL×2) and dried over Na₂SO₄. The filtrate was concentrated under reduced pressure to afford (C44) (650 mg, 2.29 mmol, 68% yield) as a white solid. ¹H NMR (400 MHz, ((CD₃)₂SO)): δ 7.76-7.74 (m, 1H), 5.83-5.66 (m, 1H), 4.08-3.50 (m, 5H), 2.46-2.28 (m, 2H), 0.73-0.68 (m, 4H).

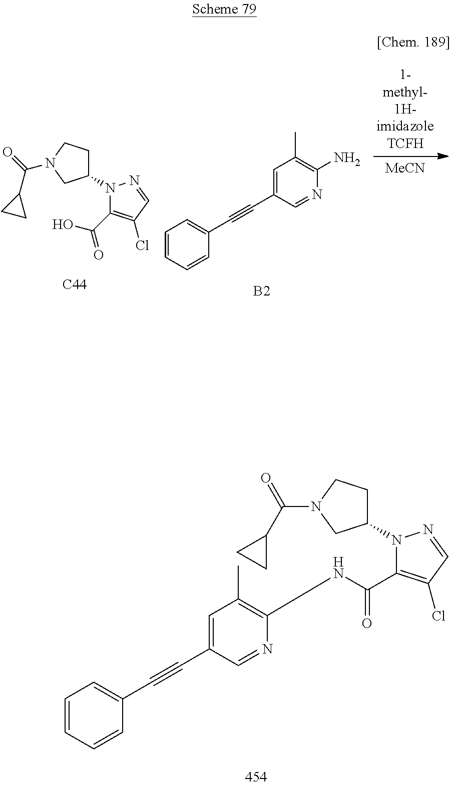

4-chloro-1-[(3S)-1-(cyclopropanecarbonyl)pyrrolidin-3-yl]-N-[3-methyl-5-(phenylethynyl)pyridin-2-yl]-1H-pyrazole-5-carboxamide (454): To a solution of (C44) (200 mg, 0.70 mmol) in MeCN (5 mL) was added (B2) (73.4 mg, 0.35 mmol) and 1-methyl-1H-imidazole (0.2 mL, 2.47 mmol) and N-(chloro(dimethylamino)methylene)-N-methylmethanaminium hexafluorophosphate(V) (395.6 mg, 1.41 mmol) at 20° C. After the resulting mixture was stirred at 20° C. for 4 h, the reaction mixture was concentrated. To the residue was added MeOH (1 mL) and 1 mon/L NaOH-aq (1 mL) was added and the mixture was stirred at 50° C. for 4 h. The reaction mixture was poured into water (5 mL) and extracted with DCM (5 mL×4). The combined organic layers were washed with brine (5 mL×2) and dried over Na₂SO₄. The filtrate was concentrated under reduced pressure. The residue was purified by prep-HPLC (column:Welch Xtimate C18 150*25 mm*5 um) (water (containing 0.2% FA)/MeCN=55/45-15/85). The afforded flows were combined, concentrated to remove most of CH₃CN and lyophilized to afford (454) (52.4 mg, 0.11 mmol, 16% yield) as a yellow solid. *¹ESI-MS[M+1]⁺: 474.2, LCMS Retention time: 1.20 min, ¹H NMR (400 MHz, CDCl₃): δ 8.61 (m, 1H), 8.51 (m, 1H), 7.77 (m, 1H), 7.58-7.54 (m, 3H), 7.39 (m, 3H), 5.95 (m, 1H), 4.18-3.65 (m, 5H), 2.60-2.40 (m, 2H), 2.39-2.36 (m, 3H), 1.02-0.95 (m, 2H), 0.80-0.74 (m, 2H). *1: The data were measured by Reversed-phase LCMS method (3) as described above.

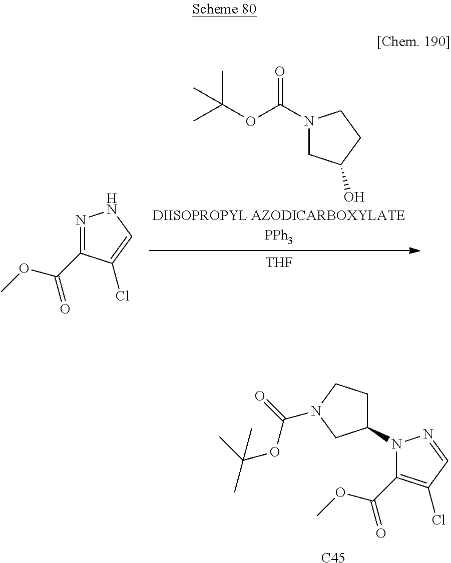

Methyl (R)-1-(1-(tert-butoxycarbonyl)pyrrolidin-3-yl)-4-chloro-1H-pyrazole-5-carboxylate (C45): To a solution of (methyl 4-chloro-1H-pyrazole-5-carboxylate) (1.0 g, 6.23 mmol), ((S)-tert-butyl 3-hydroxypyrrolidine-1-carboxylate) (1220 mg, 6.54 mmol) and PPh₃ (2.12 g, 8.10 mmol) in THF (6 mL) was added DIISOPROPYL AZODICARBOXYLATE (1640 mg, 8.10 mmol) at 10 degrees. The reaction mixture was stirred at room temperature for 1 hr. To the reaction mixture was added EtOAc, H2O, and then brine. The aqueous mixture was extracted with EtOAc, and then organic layer was washed with brine, dried over Na₂SO₄, filtered to remove inorganics, and concentrated. The residue was purified by SiO₂ column chromatography (nHexane/EtOAc=90/10 15/85) to give (C45) (1.73 g, 84% yield) as a colorless oil. *¹ESI-MS[M-tBu+2H]⁺: 274, ¹H NMR (400 MHz, ((CD₃)₂SO): δ 7.48 (m, 1H), 5.71 (m, 1H), 3.95 (s, 3H), 3.79 (m, 1H), 3.69-3.61 (m, 2H), 3.50 (m, 1H), 2.45 (m, 1H), 2.30 (m, 1H), 1.45 (s, 9H).*¹: The data were measured by Reversed-phase LCMS method (3) as described above.

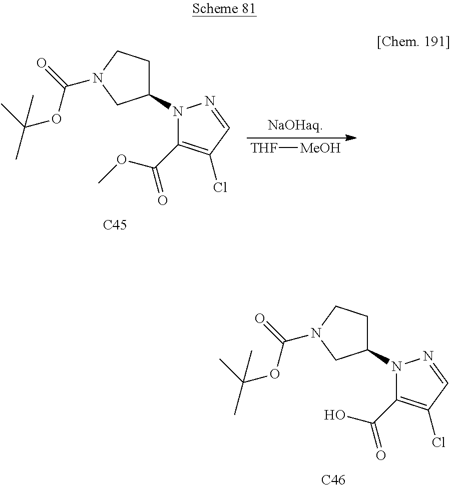

(R)-1-(1-(tert-butoxycarbonyl)pyrrolidin-3-yl)-4-chloro-1H-pyrazole-5-carboxylic acid (C46): To a solution of (C45) (1.66 g, 5.28 mmol) in THF-MeOH (6.68 mL-6.68 mL) was added 2 mon/L NaOH-aq. (6.3 mL, 12.7 mmol) and the reaction mixture was stirred at room temperature for 45 min. To the reaction mixture was added 1 mol/L HCl-aq. (15 mL), and the aqueous mixture was extracted with EtOAc, and then organic layer was washed with brine, dried over Na₂SO₄ and concentrated to give (C46) (1.67 g, quant.) as a colorless oil. *¹ESI-MS [M-tBu+2H]⁺: 260, ¹H NMR (400 MHz, ((CD₃)₂SO)): δ 7.75 (m, 1H), 5.67 (m, 1H), 3.68 (m, 1H), 3.53-3.25 (m, 3H), 2.38-2.20 (m, 2H), 1.40-1.37 (m, 9H). *¹: The data were measured by Reversed-phase LCMS method (3) as described above.

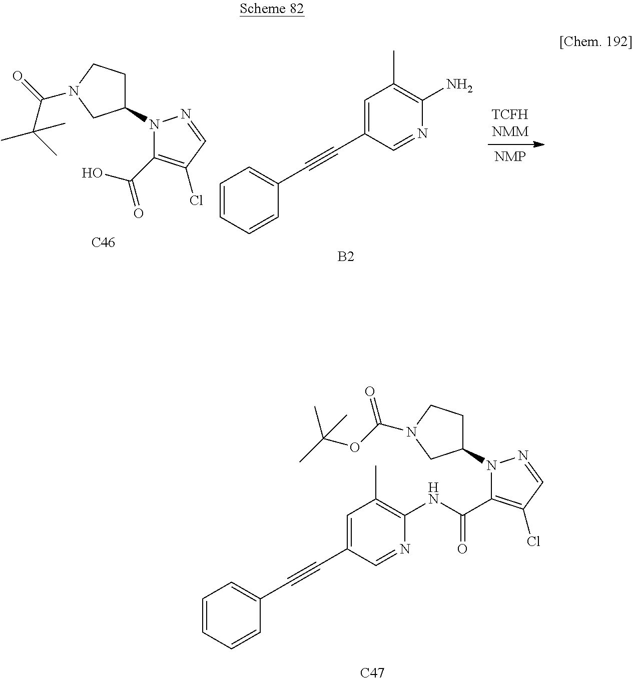

tert-butyl (R)-3-(4-chloro-5-((3-methyl-5-(phenylethynyl)pyridin-2-yl)carbamoyl)-1H-pyrazol-1-yl)pyrrolidine-1-carboxylate (C47): To a solution of (C46) (400 mg, 1.27 mmol) and (B2) (277 mg, 1.33 mmol) in NMP (3.2 mL) was added 4-METHYLMORPHOLINE (0.28 mL, 2.53 mmol). To the reaction mixture was added CHLORO-N,N,N′,N′-TETRAMETHYLFORMAMIDINIUM HEXAFLUOROPHOSPHATE (533 mg, 1.90 mmol) at 0 degrees. The resulting mixture was stirred at room temperature for 2.5 h. To the reaction mixture was added EtOAc, H₂O and then 1 mol/L HCl-aq. The organic layer was separated and washed with sat. NaHCO₃-aq and brine, dried over Na₂SO₄, filtered and concentrated under reduced pressure. The residue was purified by SiO₂ column chromatography (nHexane/EtOAc=92/8-65/35) to give (C47) (174 mg, 27%) as a yellow amorphous powder. *¹ESI-MS[M+1]⁺: 506 (M+H), 1 H NMR (400 MHz, ((CD₃)₂SO)): δ 10.98 (brs, 1H), 8.40 (brs, 1H), 7.95 (m, 1H), 7.70 (s, 1H), 7.60-7.57 (m, 2H), 7.46-7.39 (m, 3H), 5.25 (brs, 1H), 3.71 (m, 1H), 3.60 (m, 1H), 3.52-3.25 (m, 2H), 2.38-2.25 (m, 5H), 1.40 (s, 9H). *1: The data were measured by Reversed-phase LCMS method (3) as described above.

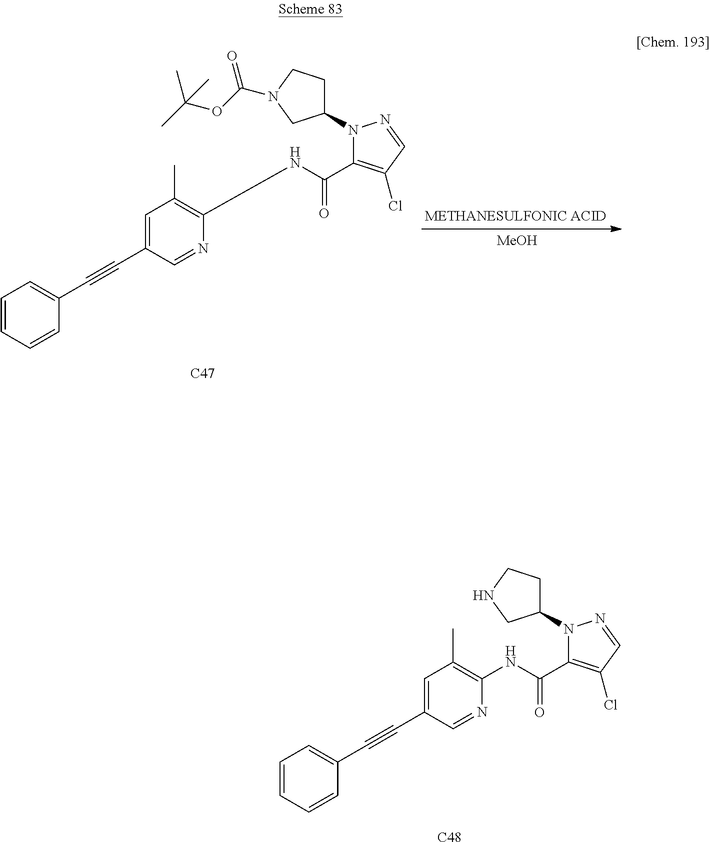 [SMILES: "MeOH" ; Cc1cc(C#Cc2ccccc2)cnc1NC(=O)c1c(Cl)cnn1[C@@H]1CCN(C(=O)OC(C)(C)C)C1 ; "C47" ; Cc1cc(C#Cc2ccccc2)cnc1NC(=O)c1c(Cl)cnn1[C@@H]1CCNC1 ; "C48"]

(R)-4-chloro-N-(3-methyl-5-(phenylethynyl)pyridin-2-yl)-1-(pyrrolidin-3-yl)-1H-pyrazole-5-carboxamide (C48): To a solution of (C47) (163 mg, 0.322 mmol) in MeOH (1.2 mL) was added METHANESULFONIC ACID (0.084 mL, 1.29 mmol). The resulting mixture was stirred at 60° C. for 4 h. The reaction mixture was added to NaHCO₃-aq and extracted with EtOAc. The organic layer was washed with brine, dried over Na₂SO₄, filtered and concentrated under reduced pressure to afford (C48) (129 mg, 99% yield) as a yellow amorphous powder. *¹ ESI-MS [M+1]⁺: 406, 1H NMR (400 MHz, ((CD₃)₂SO)): δ8.41 (m, 1H), 7.96 (m, 1H), 7.65 (s, 1H), 7.60-7.54 (m, 2H), 7.48-7.42 (m, 3H), 5.06 (m, 1H), 3.25-2.80 (m, 4H), 2.29 (s, 3H), 2.24-2.08 (m, 2H). *¹: The data were measured by Reversed-phase LCMS method (3) as described above.

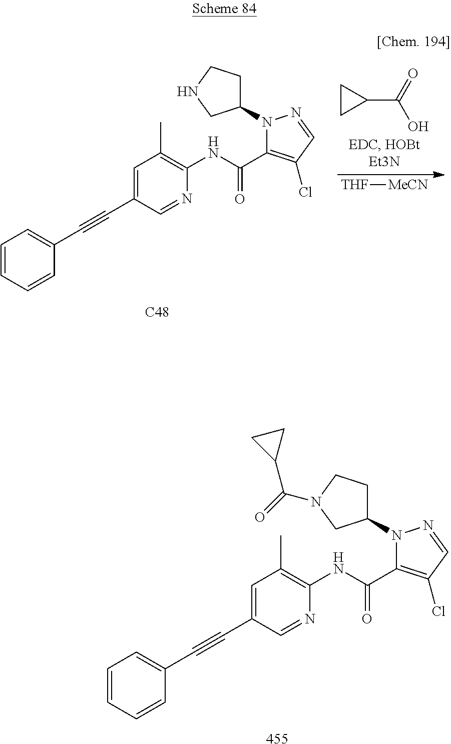

4-chloro-1-[(3R)-1-(cyclopropanecarbonyl)pyrrolidin-3-yl]-N-[3-methyl-5-(phenylethynyl)pyridin-2-yl]-1H-pyrazole-5-carboxamide (455): To a solution of (C48) (60 mg, 0.148 mmol) in THF-MeCN (0.6 mL-0.6 mL) was added cyclopropanecarboxylic acid (12.7 mg, 0.148 mmol), Et₃N (0.0247 mL, 0.177 mmol), 1-HYDROXYBENZOTRIAZOLE MONOHYDRATE (24.9 mg, 0.163 mmol) and EDC-HCl (31.2 mg, 0.163 mmol). The reaction mixture was stirred at room temperature for 3 h. To the reaction mixture was poured into lmol/L HCl-aq., and extracted with EtOAc. The organic layer was washed with sat. NaHCO₃-aq., brine, dried over Na₂SO₄, filtered and concentrated under reduced pressure. The residue was purified by SiO₂ column chromatography (nHexane/EtOAc=50/50-0/100) to give (455) (56 mg, 80% yield) as a pale yellow amorphous powder. *¹ESI-MS[M+1]⁺: 474.2, LCMS Retention time: 1.20 min, ¹H NMR (400 MHz, ((CD₃)₂SO)): δ 11.00 (brs, 1H), 8.40 (brs, 1H), 7.96 (brs, 1H), 7.70 (m, 1H), 7.62-7.52 (m, 2H), 7.50-7.39 (m, 3H), 5.45-5.22 (m, 1H), 4.22-3.20 (m, 5H), 2.38-2.30 (m, 2H), 2.31 (m, 3H), 0.80-0.68 (m, 4H).*1: The data were measured by Reversed-phase LCMS method (3) as described above.

The compounds shown in Table 1 were prepared in an analogous manner with the appropriate starting materials, and evaluated in our TREK-1 Thallium (Tl⁺) flux assay.

TABLE 1

| | | | | Tl⁺ |
|---|---|---|---|---|
| | | | | Assay |
| Cpd. | | | ES-MS | IC₅₀ |
| No. | Name | Structure | [M + 1]⁺ | (μM) |
| 1 | 1-((1,4-dioxan-2-yl)methyl-4-chloro- N-(3-methyl-5- (phenylethynyl)pyridin-2-yl)-1H- pyrazole-5-carboxamide | 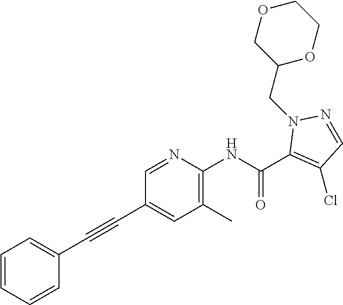 [SMILES: Cc1cc(C#Cc2ccccc2)cnc1NC(=O)c1c(Cl)cnn1CC1COCCO1] | 437 | 0.67 |
| 2 | 4-chloro-N-(3-methyl-5- (phenylethynyl)pyridin-2-yl)-1- ((tetrahydro-2H-pyran-4-yl)methyl)- 1H-pyrazole-5-carboxamide | 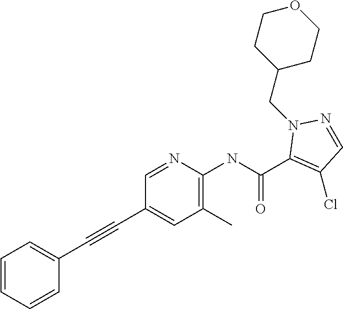 [SMILES: Cc1cc(C#Cc2ccccc2)cnc1NC(=O)c1c(Cl)cnn1CC1CCOCC1] | 435 | 0.20 |
| 3 | (S)-4-chloro-N-(3-methyl-5- (phenylethynyl)pyridin-2-yl)-1- (tetrahydrofuran-3-yl)-1H-pyrazole-5- carboxamide | 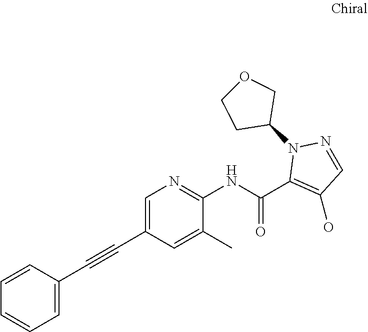 [SMILES: Cc1cc(C#Cc2ccccc2)cnc1NC(=O)c1c(O)cnn1[C@H]1CCOC1] | 407.4 | 0.36 |
| 4 | (S)-1-((1,4-dioxan-2-yl)methyl)-4- chloro-N-(3-fluoro-5- (phenylethynyl)pyridin-2-yl)-1H- pyrazole-5-carboxamide | 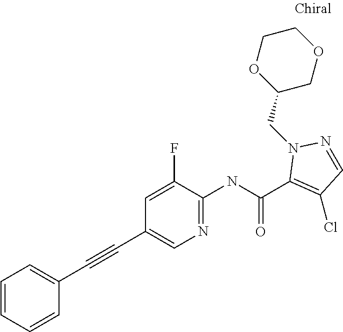 [SMILES: O=C(Nc1ncc(C#Cc2ccccc2)cc1F)c1c(Cl)cnn1C[C@H]1COCCO1] | 441 | 0.22 |
| 5 | 1-(1-acetylpiperidin-4-yl)-4-chloro-N- (3-methyl-5-(phenylethynyl)pyridin-2- yl)-1H-pyrazole-5-carboxamide | 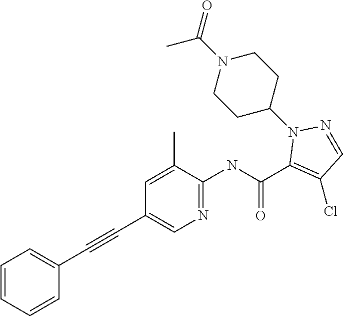 [SMILES: CC(=O)N1CCC(n2ncc(Cl)c2C(=O)Nc2ncc(C#Cc3ccccc3)cc2C)CC1] | 462 | 0.07 |
| 6 | 1-(1-acetylpiperidin-4-yl)-4-chloro-N- (3-fluoro-5-(phenylethynyl)pyridin-2- yl)-1H-pyrazole-5-carboxamide | 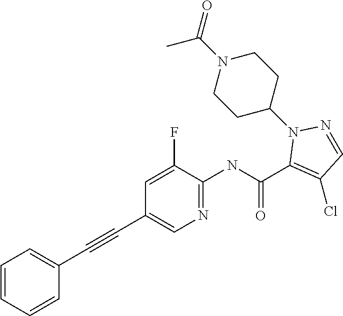 [SMILES: CC(=O)N1CCC(n2ncc(Cl)c2C(=O)Nc2ncc(C#Cc3ccccc3)cc2F)CC1] | 466 | 0.07 |
| 7 | 1-(1-acetylpiperidin-4-yl)-4-chloro-N- (3-fluoro-5-((4- fluorophenyl)ethynyl)pyridin-2-yl)- 1H-pyrazole-5-carboxamide | 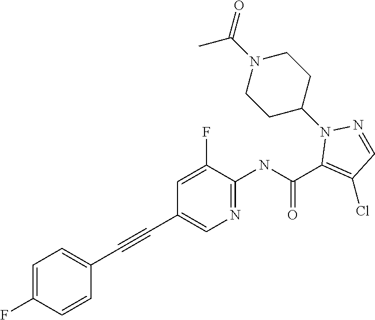 [SMILES: CC(=O)N1CCC(n2ncc(Cl)c2C(=O)Nc2ncc(C#Cc3ccc(F)cc3)cc2F)CC1] | 483.9 | 0.14 |
| 8 | 1-(1-acetylpiperidin-4-yl)-4-chloro-N- (5-((4-fluorophenyl)ethynyl)-3- methylpyridin-2-yl)-1H-pyrazole-5- carboxamide | 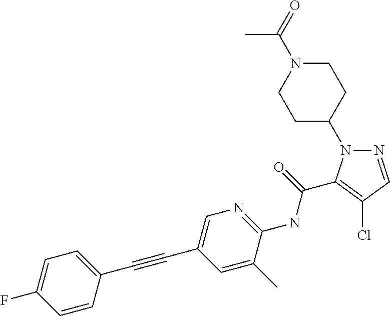 [SMILES: CC(=O)N1CCC(n2ncc(Cl)c2C(=O)Nc2ncc(C#Cc3ccc(F)cc3)cc2C)CC1] | 480.4 | 0.20 |
| 9 | 4-chloro-N-(3-methyl-5- (phenylethynyl)pyridin-2-yl)-1- (4,5,6,7-tetrahydropyrazolo[1,5- a]pyridin-5-yl)-1H-pyrazole-5- carboxamide | 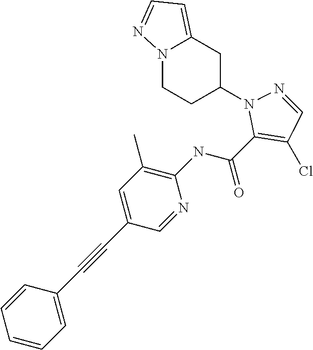 [SMILES: Cc1cc(C#Cc2ccccc2)cnc1NC(=O)c1c(Cl)cnn1C1CCn2nccc2C1] | 457 | 0.14 |
| 10 | 1-((3R,4R)-1-acetyl-3-fluoropiperidin- 4-yl)-4-chloro-N-(5-((4- fluorophenyl)ethynyl)-3- methylpyridin-2-yl)-1H-pyrazole-5- carboxamide | 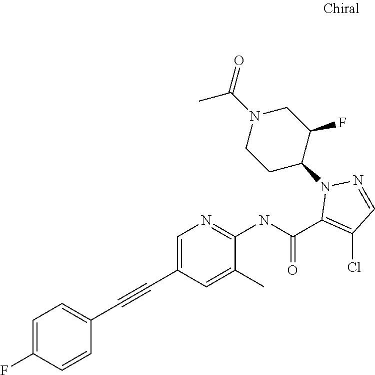 [SMILES: CC(=O)N1CC[C@H](n2ncc(Cl)c2C(=O)Nc2ncc(C#Cc3ccc(F)cc3)cc2C)[C@H](F)C1] | 498.3 | 0.11 |
| 11 | 1-(1-(1,4-dioxane-2- carbonyl)piperidin-4-yl)-4-chloro-N- (3-fluoro-5-(phenylethynyl)pyridin-2- yl)-1H-pyrazole-5-carboxamide | 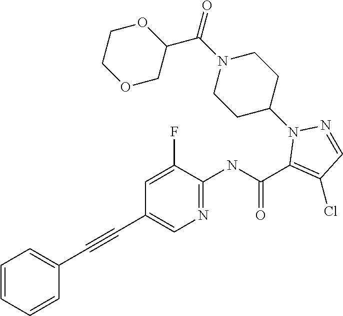 [SMILES: O=C(Nc1ncc(C#Cc2ccccc2)cc1F)c1c(Cl)cnn1C1CCN(C(=O)C2COCCO2)CC1] | 538 | 0.12 |
| 12 | 1-(cis-4-acetamidocyclohexyl)-4- chloro-N-(3-methyl-5- (phenylethynyl)pyridin-2-yl)-1H- pyrazole-5-carboxamide | 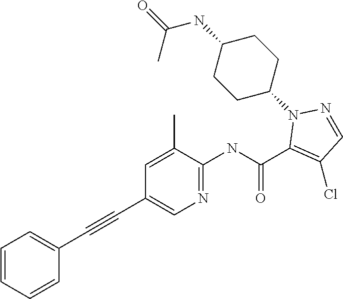 [SMILES: CC(=O)N[C@H]1CC[C@@H](n2ncc(Cl)c2C(=O)Nc2ncc(C#Cc3ccccc3)cc2C)CC1] | 476.3 | 0.13 |
| 13 | 1-(cis-4-acetamidocyclohexyl)-4- chloro-N-(3-fluoro-5- (phenylethynyl)pyridin-2-yl)-1H- pyrazole-5-carboxamide | 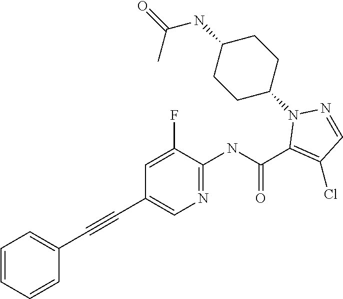 [SMILES: CC(=O)N[C@H]1CC[C@@H](n2ncc(Cl)c2C(=O)Nc2ncc(C#Cc3ccccc3)cc2F)CC1] | 480.3 | 0.12 |
| 14 | 1-(cis-4-acetamidocyclohexyl)-4- chloro-N-(5-((4- fluorophenyl)ethynyl)-3- methylpyridin-2-yl)-1H-pyrazole-5- carboxamide | 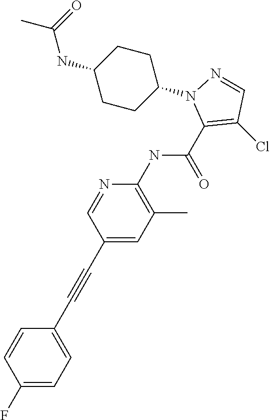 [SMILES: CC(=O)N[C@H]1CC[C@@H](n2ncc(Cl)c2C(=O)Nc2ncc(C#Cc3ccc(F)cc3)cc2C)CC1] | 494.3 | 0.26 |
| 15 | 1-((1-acetyl-4-fluoropiperidin-4- yl)methyl)-4-chloro-N-(3-methyl-5- (phenylethynyl)pyridin-2-yl)-1H- pyrazole-5-carboxamide | 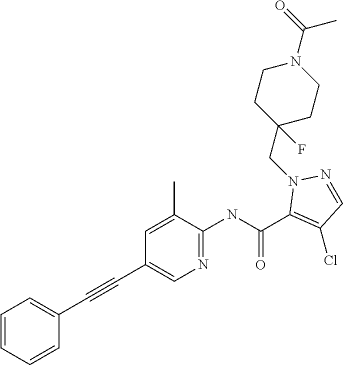 [SMILES: CC(=O)N1CCC(F)(Cn2ncc(Cl)c2C(=O)Nc2ncc(C#Cc3ccccc3)cc2C)CC1] | 494.3 | 0.4 |
| 16*² | 1-[(1R)-1-(1-acetylpiperidin-4- yl)ethyl]-4-chloro-N-[3-methyl-5- (phenylethynyl)pyridin-2-yl]-1H- pyrazole-5-carboxamide or 1-[(1S)-1-(1-acetylpiperidin-4- yl)ethyl]-4-chloro-N-[3-methyl-5- (phenylethynyl)pyridin-2-yl]-1H- pyrazole-5-carboxamide | 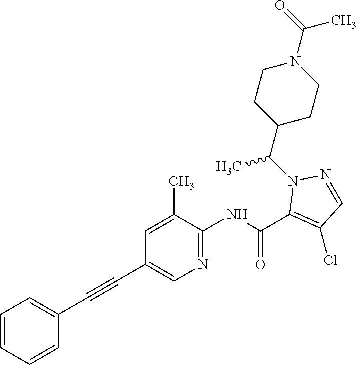 [SMILES: CC(=O)N1CCC(C(C)n2ncc(Cl)c2C(=O)Nc2ncc(C#Cc3ccccc3)cc2C)CC1] | 490.4 | 0.1 |
| 17 | 1-(1-acetylpiperidin-4-yl)-4-bromo-N- (3-methyl-5-(phenylethynyl)pyridin-2- yl)-1H-pyrazole-5-carboxamide | 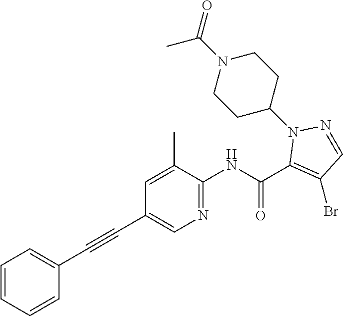 [SMILES: CC(=O)N1CCC(n2ncc(Br)c2C(=O)Nc2ncc(C#Cc3ccccc3)cc2C)CC1] | 508.3 | 0.04 |
| 18 | 1-(1-acetylpiperidin-4-yl)-4-chloro-N- (3-fluoro-5-(thiophen-2- ylethynyl)pyridin-2-yl)-1H-pyrazole- 5-carboxamide | 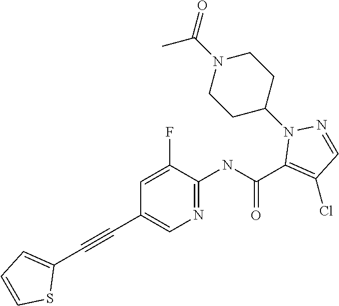 [SMILES: CC(=O)N1CCC(n2ncc(Cl)c2C(=O)Nc2ncc(C#Cc3cccs3)cc2F)CC1] | 471.9 | 0.06 |
| 19 | 1-(1-acetylpiperidin-4-yl)-4-chloro-N- (3-methyl-5-(thiophen-2- ylethynyl)pyridin-2-yl)-1H-pyrazole- 5-carboxamide | 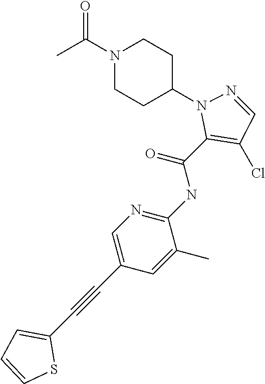 [SMILES: CC(=O)N1CCC(n2ncc(Cl)c2C(=O)Nc2ncc(C#Cc3cccs3)cc2C)CC1] | 468.3 | 0.06 |
| 20 | 1-((3R,4R)-1-acetyl-3-fluoropiperidin- 4-yl)-4-chloro-N-(3-fluoro-5- (phenylethynyl)pyridin-2-yl)-1H- pyrazole-5-carboxamide | 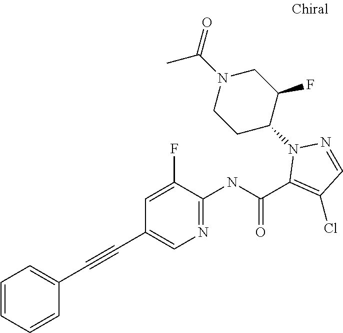 [SMILES: CC(=O)N1CC[C@@H](n2ncc(Cl)c2C(=O)Nc2ncc(C#Cc3ccccc3)cc2F)[C@H](F)C1] | 484.2 | 0.07 |
| 21 | 4-chloro-1-(2,3- dihydrobenzo[b][1,4]dioxin-6-yl)-N- (3-methyl-5-(phenylethynyl)pyridin-2- yl)-1H-pyrazole-5-carboxamide | 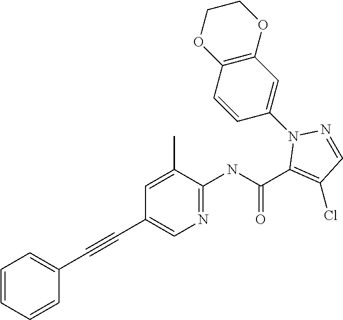 [SMILES: Cc1cc(C#Cc2ccccc2)cnc1NC(=O)c1c(Cl)cnn1-c1ccc2c(c1)OCCO2] | 471 | 0.07 |
| 22 | 4-chloro-1-(1-methyl-1H-indazol-5- yl)-N-(3-methyl-5- (phenylethynyl)pyridin-2-yl)-1H- pyrazole-5-carboxamide | 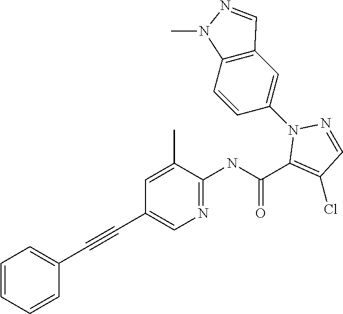 [SMILES: Cc1cc(C#Cc2ccccc2)cnc1NC(=O)c1c(Cl)cnn1-c1ccc2c(cnn2C)c1] | 467.2 | 0.08 |
| 23 | 1-(cis-4-(1,4-dioxane-2- carboxamido)cyclohexyl)-4-chloro-N- (3-fluoro-5-((4- fluorophenyl)ethynyl)pyridin-2-yl)- 1H-pyrazole-5-carboxamide | 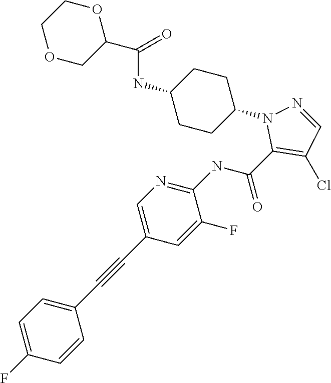 [SMILES: O=C(Nc1ncc(C#Cc2ccc(F)cc2)cc1F)c1c(Cl)cnn1[C@H]1CC[C@@H](NC(=O)C2COCCO2)CC1] | 570.2 | 0.08 |
| 24 | 1-((3S,4R)-1-acetyl-3-fluoropiperidin- 4-yl)-4-chloro-N-(3-methyl-5- (phenylethynyl)pyridin-2-yl)-1H- pyrazole-3-carboxamide | 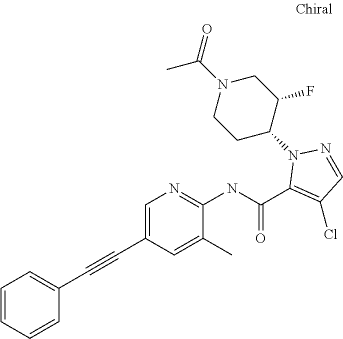 [SMILES: CC(=O)N1CC[C@@H](n2ncc(Cl)c2C(=O)Nc2ncc(C#Cc3ccccc3)cc2C)[C@@H](F)C1] | 480.2 | 0.08 |
| 25 | 1-(1-acetylpiperidin-4-yl)-4-chloro-N- (3-fluoro-5-((3- fluorophenyl)ethynyl)pyridin-2-yl)- 1H-pyrazole-5-carboxamide | 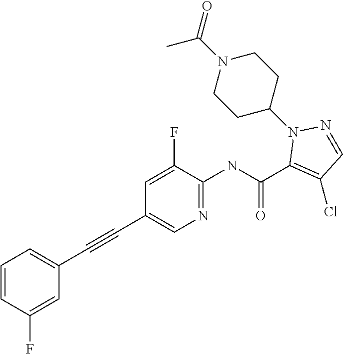 [SMILES: CC(=O)N1CCC(n2ncc(Cl)c2C(=O)Nc2ncc(C#Cc3cccc(F)c3)cc2F)CC1] | 483.8 | 0.09 |
| 26 | 1-(1-acetylpiperidin-4-yl)-4-chloro-N- (3-fluoro-5-(phenylethynyl)pyridin-2- yl)-1H-pyrazole-5-carboxamide | 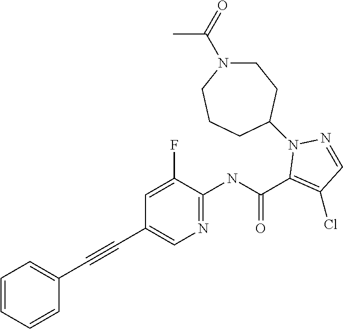 [SMILES: CC(=O)N1CCCC(n2ncc(Cl)c2C(=O)Nc2ncc(C#Cc3ccccc3)cc2F)CC1] | 480.3 | 0.09 |
| 27 | 1-((4-acetylmorpholin-2-yl)methyl)-4- chloro-N-(3-fluoro-5- (phenylethynyl)pyridin-2-yl)-1H- pyrazole-5-carboxamide | 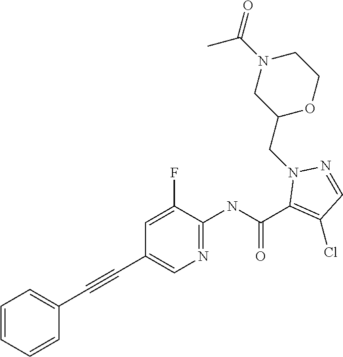 [SMILES: CC(=O)N1CCOC(Cn2ncc(Cl)c2C(=O)Nc2ncc(C#Cc3ccccc3)cc2F)C1] | 482.2 | 0.09 |
| 28 | 4-chloro-1-(1-(1-isobutyrylpiperidin- 4-yl)ethyl)-N-(3-methyl-5- (phenylethynyl)pyridin-2-yl)-1H- pyrazole-5-carboxamide | 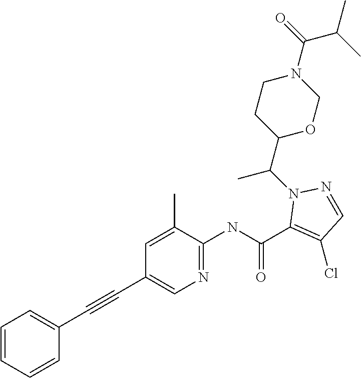 [SMILES: Cc1cc(C#Cc2ccccc2)cnc1NC(=O)c1c(Cl)cnn1C(C)C1CCN(C(=O)C(C)C)CO1] | 518.2 | 0.09 |
| 29 | 1-(1-acetyl-3-fluoropiperidin-4-yl)-4- chloro-N-(3-methyl-5- (phenylethynyl)pyridin-2-yl)-1H- pyrazole-5-carboxamide | 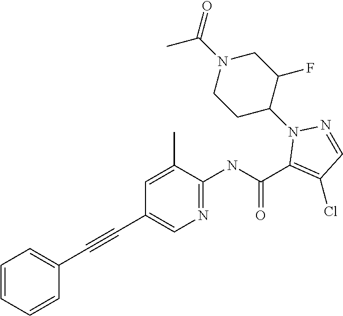 [SMILES: CC(=O)N1CCC(n2ncc(Cl)c2C(=O)Nc2ncc(C#Cc3ccccc3)cc2C)C(F)C1] | 480.3 | 0.1 |
| 30 | 1-((3R,4R)-1-acetyl-5-fluoropiperidin- 4-yl)-4-chloro-N-(3-methyl-5- (phenylethynyl)pyridin-2-yl)-1H- pyrazole-5-carboxamide | 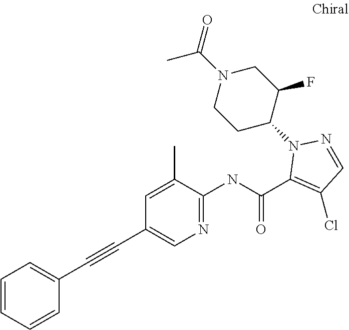 [SMILES: CC(=O)N1CC[C@@H](n2ncc(Cl)c2C(=O)Nc2ncc(C#Cc3ccccc3)cc2C)[C@H](F)C1] | 480.3 | 0.1 |
| 31*⁵ | 1-[(2S,4S)-1-acetyl-2-methylpiperidin- 4-yl]-4-chloro-N-[3-methyl-5- (phenylethynyl)pyridin-2-yl]-1H- pyrazole-5-carboxamide or 1-[(2R,4R)-1-acetyl-2- methylpiperidin-4-yl]-4-chloro-N-[3- methyl-5-(phenylethynyl)pyridin-2- yl]-1H-pyrazole-5-carboxamide | 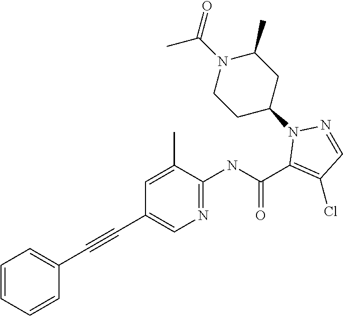 [SMILES: CC(=O)N1CC[C@H](n2ncc(Cl)c2C(=O)Nc2ncc(C#Cc3ccccc3)cc2C)C[C@@H]1C] | 476.3 | 0.1 |
| | | 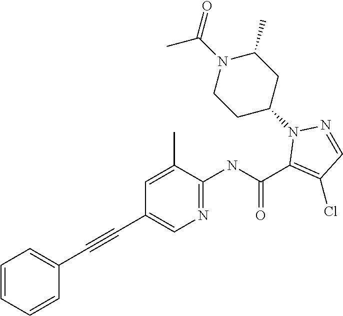 [SMILES: CC(=O)N1CC[C@@H](n2ncc(Cl)c2C(=O)Nc2ncc(C#Cc3ccccc3)cc2C)C[C@H]1C] | | |
| 32 | 1-(1-(1-acetylpiperidin-4-yl)ethyl)-4- chloro-N-(3-methyl-5- (phenylethynyl)pyridin-2-yl)-1H- pyrazole-5-carboxamide | 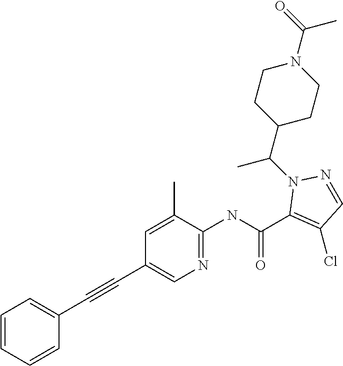 [SMILES: CC(=O)N1CCC(C(C)n2ncc(Cl)c2C(=O)Nc2ncc(C#Cc3ccccc3)cc2C)CC1] | 490.2 | 0.1 |
| 33 | 1-(1-acetylpiperidin-4-yl)-4-chloro-N- (2,6-difluoro-4- (phenylethynyl)phenyl)-1H-pyrazole- 5-carboxamide | 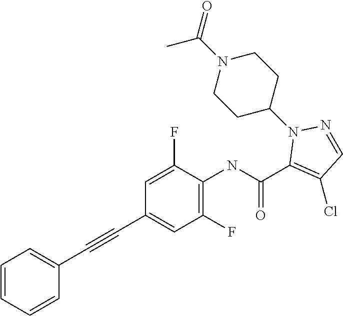 [SMILES: CC(=O)N1CCC(n2ncc(Cl)c2C(=O)Nc2c(F)cc(C#Cc3ccccc3)cc2F)CC1] | 483 | 0.11 |
| 34 | 4-chloro-N-(3-fluoro-5- (phenylethynyl)pyridin-2-yl)-1-(1- propionylpiperidin-4-yl)-1H-pyrazole- 5-carboxamide | 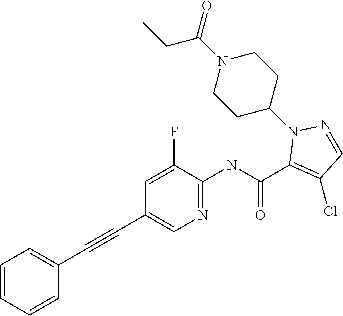 [SMILES: CCC(=O)N1CCC(n2ncc(Cl)c2C(=O)Nc2ncc(C#Cc3ccccc3)cc2F)CC1] | 480.3 | 0.11 |
| 35 | 1-(1-(1-acetylpiperidin-4-yl)ethyl)-4- chloro-N-(3-fluoro-5- (phenylethynyl)pyridin-2-yl)-1H- pyrazole-5-carboxamide | 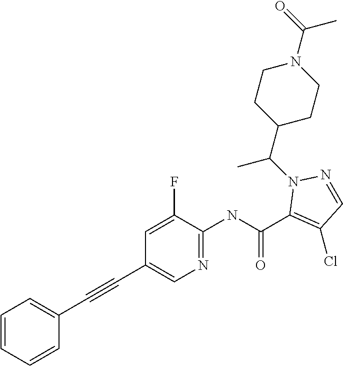 [SMILES: CC(=O)N1CCC(C(C)n2ncc(Cl)c2C(=O)Nc2ncc(C#Cc3ccccc3)cc2F)CC1] | 494.2 | 0.11 |
| 36 | 4-chloro-1-(2-(cyclopropanecarbonyl)- 2-azaspiro[3.3]heptan-6-yl)-N-(3- methyl-5-(thiophen-2- ylethynyl)pyridin-2-yl)-1H-pyrazole 5-carboxamide | 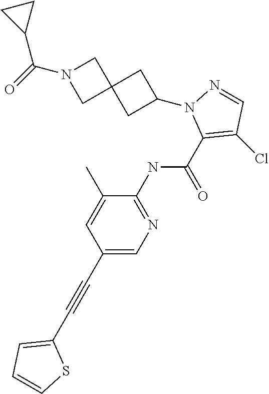 [SMILES: Cc1cc(C#Cc2cccs2)cnc1NC(=O)c1c(Cl)cnn1C1CC2(C1)CN(C(=O)C1CC1)C2] | 506.4 | 0.11 |
| 37 | 1-(1-acetylpiperidin-4-yl)-4-chloro-N- (2-fluoro-6-methyl-4- (phenylethynyl)phenyl)-1H-pyrazole- 5-carboxamide | 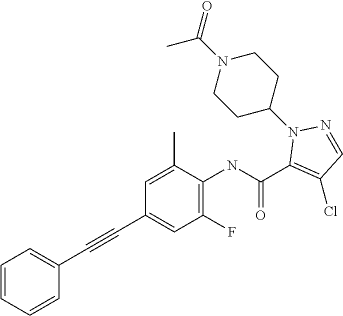 [SMILES: CC(=O)N1CCC(n2ncc(Cl)c2C(=O)Nc2c(C)cc(C#Cc3ccccc3)cc2F)CC1] | 479 | 0.12 |
| 38 | 1-(cis-4-(1,4-dioxane-2- carboxamido)cyclohexyl)-4-chloro-N- (5-((4-fluorophenyl)ethynyl)-3- methylpyridin-2-yl)-1H-pyrazole-5- carboxamide | 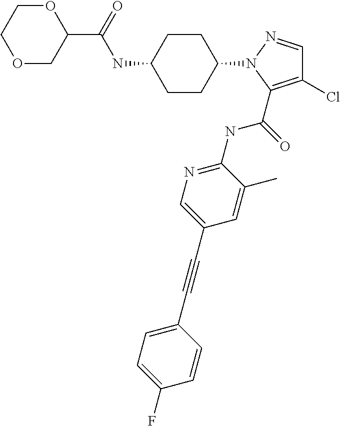 [SMILES: Cc1cc(C#Cc2ccc(F)cc2)cnc1NC(=O)c1c(Cl)cnn1[C@H]1CC[C@@H](NC(=O)C2COCCO2)CC1] | 566.3 | 0.12 |
| 39 | 1-((1-acetyl-4-cyanopiperidin-4- yl)methyl)-4-chloro-N-(3-fluoro-5- (phenylethynyl)pyridin-2-yl)-1H- pyrazole-5-carboxamide | 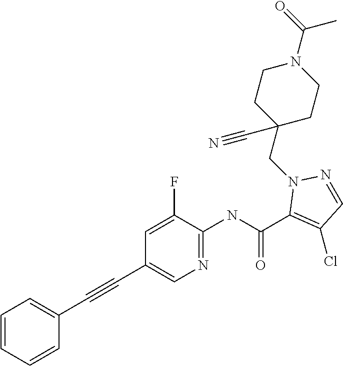 [SMILES: CC(=O)N1CCC(C#N)(Cn2ncc(Cl)c2C(=O)Nc2ncc(C#Cc3ccccc3)cc2F)CC1] | 505.3 | 0.12 |
| 40 | 1-(1-acetylazetidin-3-yl)-4-chloro-N- (3-fluoro-5-(phenylethynyl)pyridin-2- yl)-1H-pyrazole-5-carboxamide | 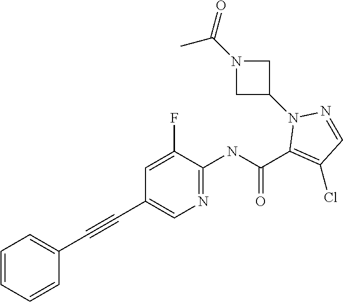 [SMILES: CC(=O)N1CC(n2ncc(Cl)c2C(=O)Nc2ncc(C#Cc3ccccc3)cc2F)C1] | 438 | 0.13 |
| 41 | 4-chloro-N-(3-methyl-5- (phenylethynyl)pyridin-2-yl)-1-(1- propionylpiperidin-4-yl)-1H-pyrazole- 5-carboxamide | 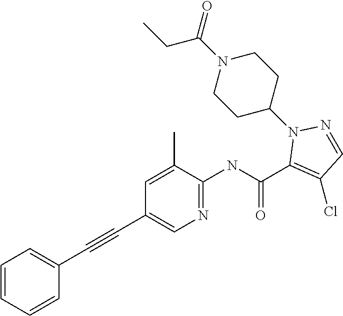 [SMILES: CCC(=O)N1CCC(n2ncc(Cl)c2C(=O)Nc2ncc(C#Cc3ccccc3)cc2C)CC1] | 476.2 | 0.13 |
| 42 | 1-((1-acetylpiperidin-4-yl)methyl)-4- chloro-N-(3-fluoro-5- (phenylethynyl)pyridin-2-yl)-1H- pyrazole-5-carboxamide | 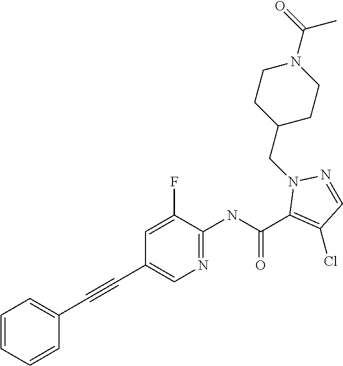 [SMILES: CC(=O)N1CCC(Cn2ncc(Cl)c2C(=O)Nc2ncc(C#Cc3ccccc3)cc2F)CC1] | 480.3 | 0.13 |
| 43 | 4-chloro-1-((1- (cyclopropanecarbonyl)pyrrolidin-3- yl)methyl)-N-(3-fluoro-5- (phenylethynyl)pyridin-2-yl)-1H- pyrazole-5-carboxamide | 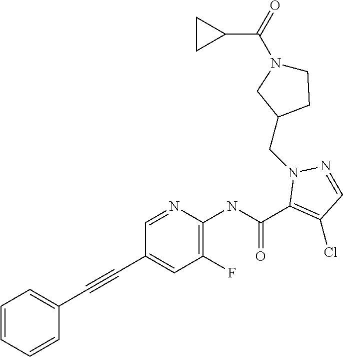 [SMILES: O=C(Nc1ncc(C#Cc2ccccc2)cc1F)c1c(Cl)cnn1CC1CCN(C(=O)C2CC2)C1] | 492.2 | 0.13 |
| 44 | 1-((1-acetylpyrrolidin-3-yl)methyl)-4- chloro-N-(3-fluoro-5- (phenylethynyl)pyridin-2-yl)-1H- pyrazole-5-carboxamide | 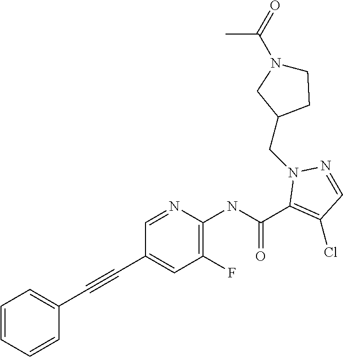 [SMILES: CC(=O)N1CCC(Cn2ncc(Cl)c2C(=O)Nc2ncc(C#Cc3ccccc3)cc2F)C1] | 466.2 | 0.13 |
| 45 | 4-chloro-N-(3-fluoro-5-((4- fluorophenyl)ethynyl)pyridin-2-yl)-1- (cis-4-isobutyramidocyclohexyl)-1H- pyrazole-5-carboxamide | 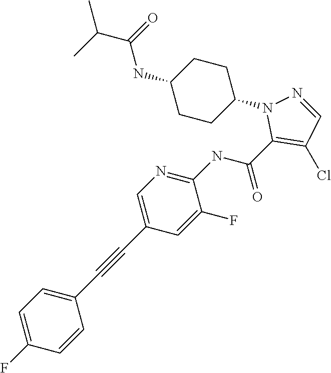 [SMILES: CC(C)C(=O)N[C@H]1CC[C@@H](n2ncc(Cl)c2C(=O)Nc2ncc(C#Cc3ccc(F)cc3)cc2F)CC1] | 526.2 | 0.13 |
| 46 | 4-chloro-1-(2-isobutyryl-2- azaspiro[3.3]heptan-6-yl)-N-(3- methyl-5-(thiophen-2- ylethynyl)pyridin-2-yl)-1H-pyrazole- 5-carboxamide | 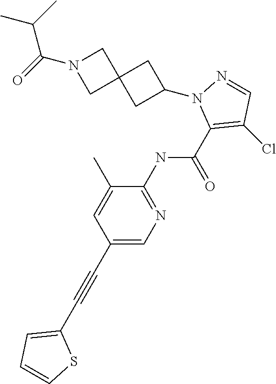 [SMILES: Cc1cc(C#Cc2cccs2)cnc1NC(=O)c1c(Cl)cnn1C1CC2(C1)CN(C(=O)C(C)C)C2] | 508.3 | 0.13 |
| 47 | 1-(trans-4-acetamidocyclohexyl)-4- chloro-N-(3-methyl-5- (phenylethynyl)pyridin-2-yl)-1H- pyrazole-5-carboxamide | 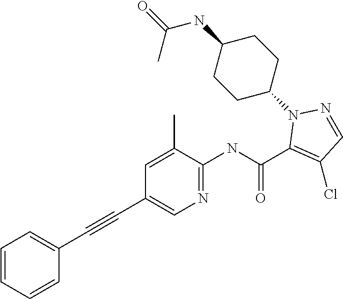 [SMILES: CC(=O)N[C@H]1CC[C@H](n2ncc(Cl)c2C(=O)Nc2ncc(C#Cc3ccccc3)cc2C)CC1] | 476.2 | 0.13 |
| 48 | 1-(((3S,4R)-1-acetyl-3- fluoropiperidin-4-yl)methyl)-4-chloro- N-(3-fluoro-5-(phenylethynyl)pyridin- 2-yl)-1H-pyrazole-5-carboxamide | 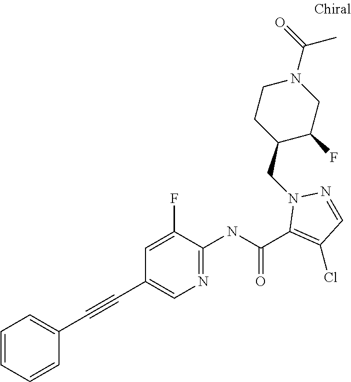 [SMILES: CC(=O)N1CC[C@H](Cn2ncc(Cl)c2C(=O)Nc2ncc(C#Cc3ccccc3)cc2F)[C@H](F)C1] | 498.3 | 0.13 |
| 49 | 4-chloro-1-((4-cyano-1- isobutyrylpiperidin-4-yl)methyl)-N-(3- methyl-5-(phenylethynyl)pyridin-2- yl)-1H-pyrazole-5-carboxamide | 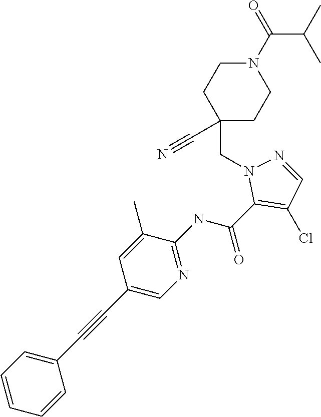 [SMILES: Cc1cc(C#Cc2ccccc2)cnc1NC(=O)c1c(Cl)cnn1CC1(C#N)CCN(C(=O)C(C)C)CC1] | 529.3 | 0.13 |
| 50 | 4-chloro-N-(3-fluoro-5-((3- fluorophenyl)ethynyl)pyridin-2-yl)-1- ((tetrahydro-2H-pyran-4-yl)methyl)- 1H-pyrazole-5-carboxamide | 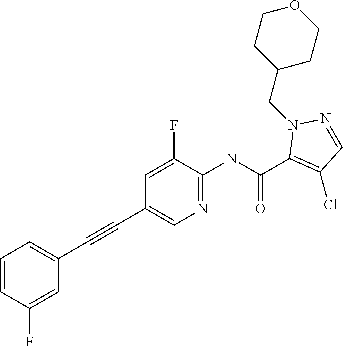 [SMILES: O=C(Nc1ncc(C#Cc2cccc(F)c2)cc1F)c1c(Cl)cnn1CC1CCOCC1] | 457 | 0.14 |
| 51 | 4-chloro-N-(3-fluoro-5- (phenylethynyl)pyridin-2-yl)-1- (tetrahydro-2H-pyran-4-yl)-1H- pyrazole-5-carboxamide | 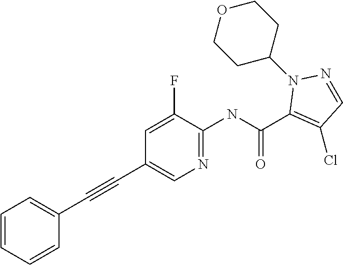 [SMILES: O=C(Nc1ncc(C#Cc2ccccc2)cc1F)c1c(Cl)cnn1C1CCOCC1] | 425 | 0.14 |
| 52 | 4-chloro-1-((1- (cyclopropanecarbonyl)piperidin-4- yl)methyl)-N-(3-methyl-5- (phenylethynyl)pyridin-2-yl)-1H- pyrazole-5-carboxamide | 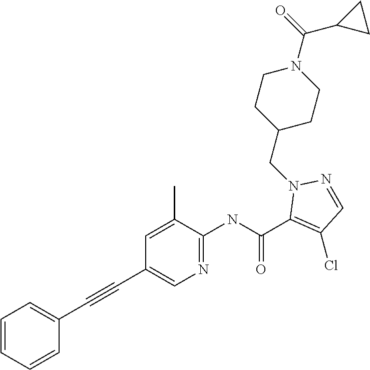 [SMILES: Cc1cc(C#Cc2ccccc2)cnc1NC(=O)c1c(Cl)cnn1CC1CCN(C(=O)C2CC2)CC1] | 502.3 | 0.14 |
| 53 | 4-chloro-1-((1-isobutyrylpiperidin-4- yl)methyl)-N-(3-methyl-5- (phenylethnyl)pyridin-2-yl)-1H- pyrazole-5-carboxamide | 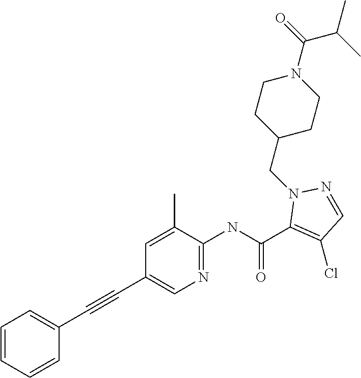 [SMILES: Cc1cc(C#Cc2ccccc2)cnc1NC(=O)c1c(Cl)cnn1CC1CCN(C(=O)C(C)C)CC1] | 504.4 | 0.14 |
| 54 | 4-chloro-N-(3-fluoro-5- (phenylethynyl)pyridin-2-yl)-1-(1- (2,2,2-trifluoroacetyl)piperidin-4-yl)- 1H-pyrazole-5-carboxamide | 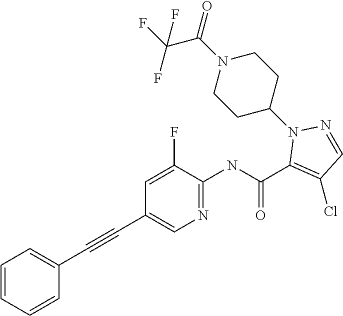 [SMILES: O=C(Nc1ncc(C#Cc2ccccc2)cc1F)c1c(Cl)cnn1C1CCN(C(=O)C(F)(F)F)CC1] | 520.2 | 0.14 |
| 55 | 4-chloro-N-(3-fluoro-5- (phenylethynyl)pyridin-2-yl)-1-(1- (3,3,3-trifluoropropanoyl)piperidin-4- yl)-1H-pyrazole-5-carboxamide | 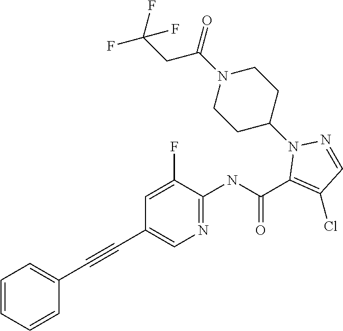 [SMILES: O=C(Nc1ncc(C#Cc2ccccc2)cc1F)c1c(Cl)cnn1C1CCN(C(=O)CC(F)(F)F)CC1] | 534.2 | 0.14 |
| 56 | 4-chloro-1-(2-(1- (cyclopropanecarbonyl)piperidin-4- yl)ethyl)-N-(3-fluoro-5- (phenylethynyl)pyridin-2-yl)-1H- pyrazole-5-carboxamide | 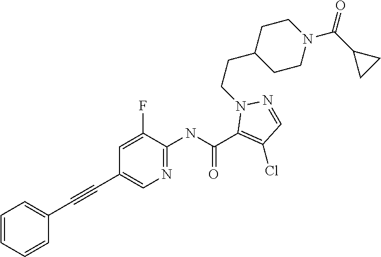 [SMILES: O=C(Nc1ncc(C#Cc2ccccc2)cc1F)c1c(Cl)cnn1CCC1CCN(C(=O)C2CC2)CC1] | 520.4 | 0.14 |
| 57 | 4-chloro-1-((1- (cyclopropanecarbonyl)piperidin-4- yl)methyl)-N-(3-methyl-5-(thiophen- 2-ylethynyl)pyridin-2-yl)-1H- pyrazole-5-carboxamide | 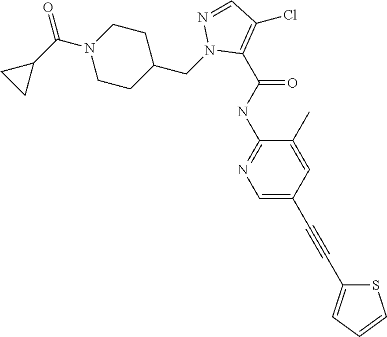 [SMILES: Cc1cc(C#Cc2cccs2)cnc1NC(=O)c1c(Cl)cnn1CC1CCN(C(=O)C2CC2)CC1] | 508.2 | 0.14 |
| 58 | 4-chloro-1-(((3S,4R)-3-fluoro-1- isobutyrylpiperidin-4-yl)methyl)-N-(3- fluoro-5-(phenylethynyl)pyridin-2-yl)- 1H-pyrazole-5-carboxamide | 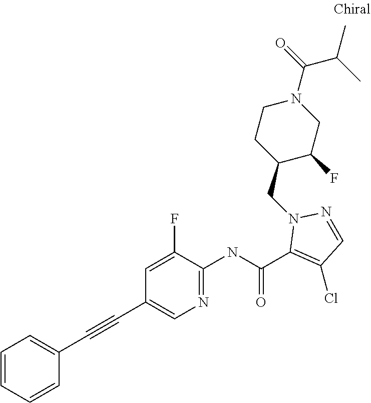 [SMILES: CC(C)C(=O)N1CC[C@H](Cn2ncc(Cl)c2C(=O)Nc2ncc(C#Cc3ccccc3)cc2F)[C@H](F)C1] | 526.3 | 0.14 |
| 59 | 4-chloro-1-(3-cyanopropyl)-N-(3- fluoro-5-(phenylethynyl)pyridin-2-yl)- 1H-pyrazole-5-carboxamide | 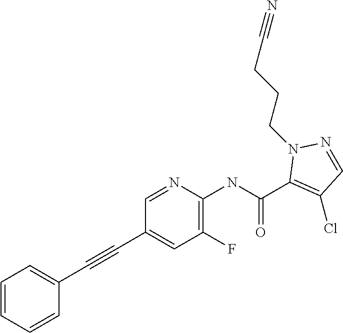 [SMILES: N#CCCCn1ncc(Cl)c1C(=O)Nc1ncc(C#Cc2ccccc2)cc1F] | 408 | 0.15 |
| 60 | (R)-4-chloro-N-(3-fluoro-5- (phenylethynyl)pyridin-2-yl)-1- ((tetrahydrofuran-3-yl)methyl)-1H- pyrazole-5-carboxamide | 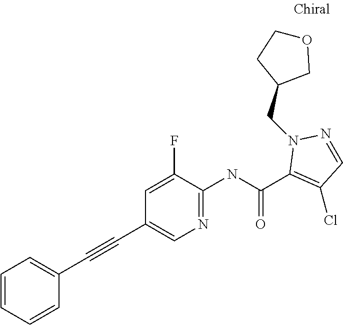 [SMILES: O=C(Nc1ncc(C#Cc2ccccc2)cc1F)c1c(Cl)cnn1C[C@H]1CCOC1] | 425 | 0.15 |
| 61 | 1-(1-acetyl-2-methylpiperidin-4-yl)-4- chloro-N-(3-methyl-5- (phenylethynyl)pyridin-2-yl)-1H- pyrazole-5-carboxamide | 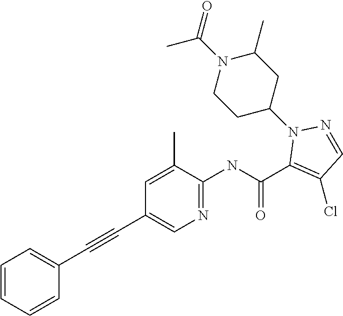 [SMILES: CC(=O)N1CCC(n2ncc(Cl)c2C(=O)Nc2ncc(C#Cc3ccccc3)cc2C)CC1C] | 476.3 | 0.15 |
| 62 | 1-(1-acetyl-2-methylpiperidin-4-yl)-4- chloro-N-(5-((4- fluorophenyl)ethynyl)-3- methylpyridin-2-yl)-1H- pyrazole-5-carboxamide | 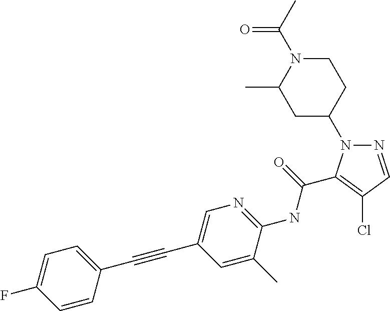 [SMILES: CC(=O)N1CCC(n2ncc(Cl)c2C(=O)Nc2ncc(C#Cc3ccc(F)cc3)cc2C)CC1C] | 494.3 | 0.15 |
| 63 | 4-chloro-1-(1- (cyclopropanecarbonyl)piperidin-4- yl)-N-(3-methyl-5-(thiophen-2- ylethynyl)pyridin-2-yl)-1H-pyrazole- 5-carboxamide | 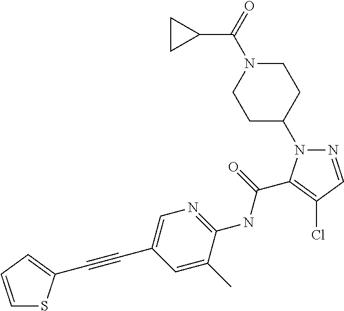 [SMILES: Cc1cc(C#Cc2cccs2)cnc1NC(=O)c1c(Cl)cnn1C1CCN(C(=O)C2CC2)CC1] | 494.3 | 0.15 |
| 64 | 1-(cis-3-acetamidocyclobutyl)-4- chloro-N-(3-methyl-5- (phenylethynyl)pyridin-2-yl)-1H- pyrazole-5-carboxamide | 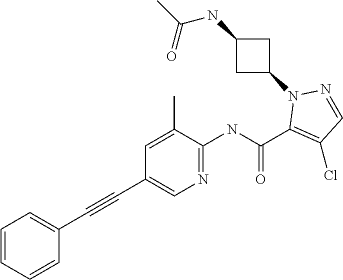 [SMILES: CC(=O)N[C@H]1C[C@@H](n2ncc(Cl)c2C(=O)Nc2ncc(C#Cc3ccccc3)cc2C)C1] | 448.4 | 0.15 |
| 65 | 1-(1-(1,4-dioxane-2- carbonyl)piperidin-4-yl)-4-chloro-N- (5-((4-fluorophenyl)ethynyl)-3- methylpyridin-2-yl)-1H-pyrazole-5- carboxamide | 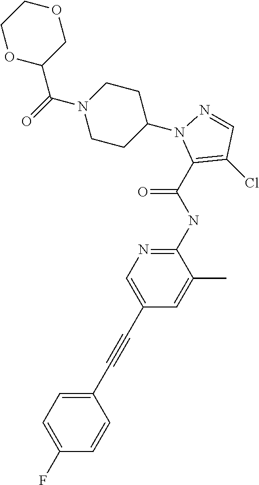 [SMILES: Cc1cc(C#Cc2ccc(F)cc2)cnc1NC(=O)c1c(Cl)cnn1C1CCN(C(=O)C2COCCO2)CC1] | 552.2 | 0.15 |
| 66 | 4-chloro-N-(5-((4- fluorophenyl)ethynyl)-3- methylpyridin-2-yl)-1-(1- propionylpiperidin-4-yl)-1H-pyrazole- 5-carboxamide | 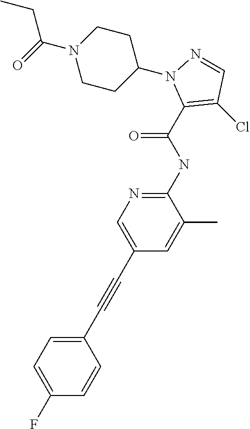 [SMILES: CCC(=O)N1CCC(n2ncc(Cl)c2C(=O)Nc2ncc(C#Cc3ccc(F)cc3)cc2C)CC1] | 494.3 | 0.15 |
| 67 | 4-chloro-N-(3-fluoro-5-(thiophen-2- ylethynyl)pyridin-2-yl)-1-(1- propionylpiperidin-4-yl)-1H-pyrazole- 5-carboxamide | 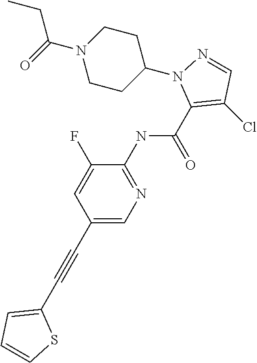 [SMILES: CCC(=O)N1CCC(n2ncc(Cl)c2C(=O)Nc2ncc(C#Cc3cccs3)cc2F)CC1] | 486.3 | 0.15 |
| 68 | 4-chloro-N-(3-fluoro-5- (phenylethynyl)pyridin-2-yl)-1- ((tetrahydro-2H-pyran-4-yl)methyl)- 1H-pyrazole-5-carboxamide | 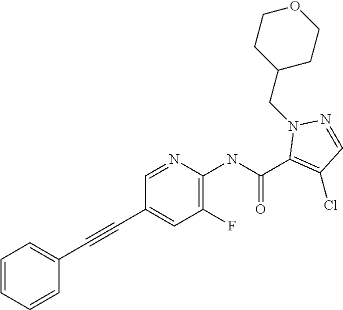 [SMILES: O=C(Nc1ncc(C#Cc2ccccc2)cc1F)c1c(Cl)cnn1CC1CCOCC1] | 439 | 0.16 |
| 69 | 4-chloro-N-(3-fluoro-5-(thiophen-2- ylethynyl)pyridin-2-yl)-1-((tetrahydro- 2H-pyran-4-yl)methyl)-1H-pyrazole- 5-carboxamide | 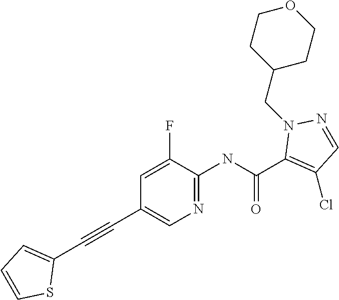 [SMILES: O=C(Nc1ncc(C#Cc2cccs2)cc1F)c1c(Cl)cnn1CC1CCOCC1] | 445 | 0.16 |
| 70 | 4-chloro-N-(3-fluoro-5- (phenylethynyl)pyridin-2-yl)-1-(1- (methylsulfonyl)piperidin-4-yl)-1H- pyrazole-5-carboxamide | 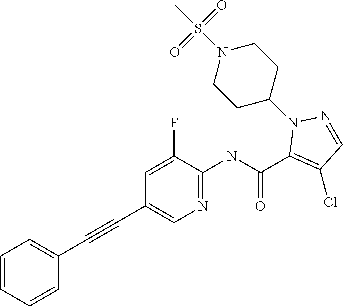 [SMILES: CS(=O)(=O)N1CCC(n2ncc(Cl)c2C(=O)Nc2ncc(C#Cc3ccccc3)cc2F)CC1] | 502 | 0.16 |
| 71 | 1-(1-acetylpiperidin-4-yl)-4-chloro-N- (5-((3-fluorophenyl)ethynyl)-3- methylpyridin-2-yl)-1H-pyrazole-5- carboxamide | 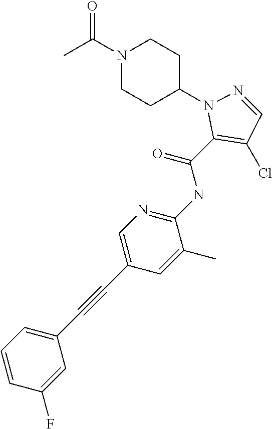 [SMILES: CC(=O)N1CCC(n2ncc(Cl)c2C(=O)Nc2ncc(C#Cc3cccc(F)c3)cc2C)CC1] | 480.3 | 0.16 |
| 72 | 1-(1-acetyl-2-methylpiperidin-4-yl)-4- chloro-N-(5-((3- fluorophenyl)ethynyl)-3- methylpyridin-2-yl)-1H-pyrazole-5- carboxamide | 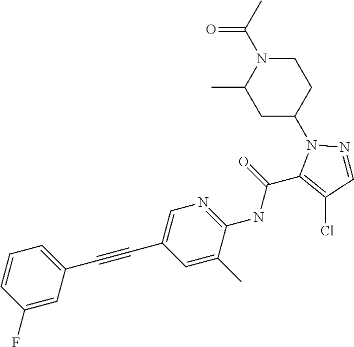 [SMILES: CC(=O)N1CCC(n2ncc(Cl)c2C(=O)Nc2ncc(C#Cc3cccc(F)c3)cc2C)CC1C] | 494.2 | 0.16 |
| 73 | 4-(4-chloro-5-((3-methyl-5-(thiophen- 2-ylethynyl)pyridin-2-yl)carbamoyl)- 1H-pyrazol-1-yl)-N-ethylpiperidine-1- carboxamide | 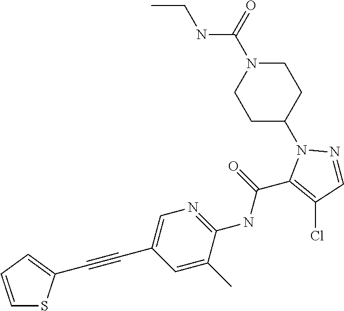 [SMILES: CCNC(=O)N1CCC(n2ncc(Cl)c2C(=O)Nc2ncc(C#Cc3cccs3)cc2C)CC1] | 497.3 | 0.16 |
| 74 | (3aR,5s,6aS)-5-[4-chloro-5-({3- methyl-5-[(thiophen-2- yl)ethynyl]pyridin-2-yl}carbamoyl)- 1H-pyrazol-1-yl]-N- ethylhexahydrocyclopenta[c]pyrrole- 2(1H)-carboxamide | 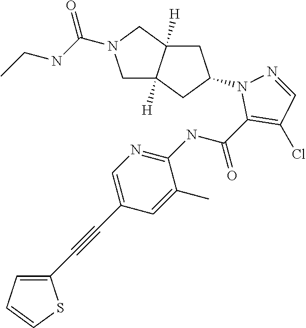 [SMILES: CCNC(=O)N1C[C@H]2C[C@H](n3ncc(Cl)c3C(=O)Nc3ncc(C#Cc4cccs4)cc3C)C[C@H]2C1] | 524.3 | 0.16 |
| 75 | 4-(4-chloro-5-((5-((3- fluorophenyl)ethynyl)-3- methylpyridin-2-yl)carbamoyl)-1H- pyrazol-1-yl)-N-ethylpiperidine-1- carboxamide | 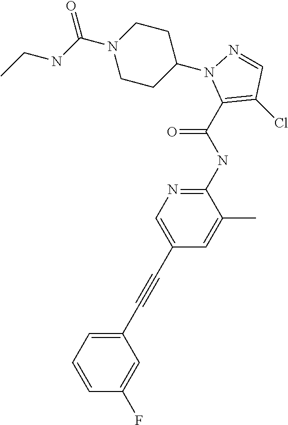 [SMILES: CCNC(=O)N1CCC(n2ncc(Cl)c2C(=O)Nc2ncc(C#Cc3cccc(F)c3)cc2C)CC1] | 509.3 | 0.16 |
| 76 | 4-chloro-1-((1-isobutyrylpiperidin-4- yl)methyl)-N-(3-methyl-5-(thiophen- 2-ylethynyl)pyridin-2-yl)-1H- pyrazole-5-carboxamide | 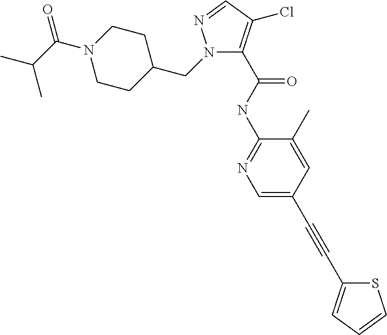 [SMILES: Cc1cc(C#Cc2cccs2)cnc1NC(=O)c1c(Cl)cnn1CC1CCN(C(=O)C(C)C)CC1] | 510.3 | 0.16 |
| 77 | tert-butyl 4-(1-(4-chloro-5-((3-methyl- 5-(phenylethynyl)pyridin-2- yl)carbamoyl)-1H-pyrazol-1- yl)ethyl)piperidine-1-carboxylate | 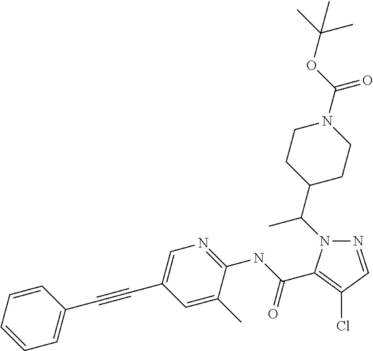 [SMILES: Cc1cc(C#Cc2ccccc2)cnc1NC(=O)c1c(Cl)cnn1C(C)C1CCN(C(=O)OC(C)(C)C)CC1] | 548.4 | 0.16 |
| 78 | tert-butyl 4-(1-(4-chloro-5-((3-fluoro- 5-(phenylethynyl)pyridin-2- yl)carbamoyl)-1H-pyrazol-1- yl)ethyl)piperidine-1-carboxylate | 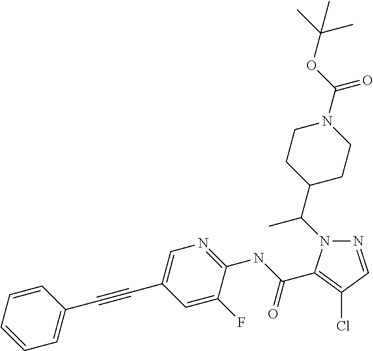 [SMILES: CC(C1CCN(C(=O)OC(C)(C)C)CC1)n1ncc(Cl)c1C(=O)Nc1ncc(C#Cc2ccccc2)cc1F] | 552.3 | 0.16 |
| 79 | 4-chloro-1-(1- (cyclopropanecarbonyl)pyrrolidin-3- yl)-N-(3-methyl-5- (phenylethynyl)pyridin-2-yl)-1H- pyrazole-5-carboxamide | 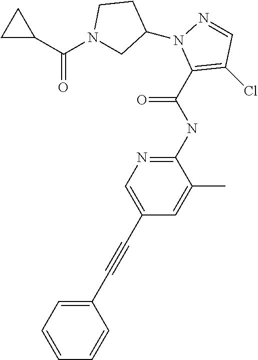 [SMILES: Cc1cc(C#Cc2ccccc2)cnc1NC(=O)c1c(Cl)cnn1C1CCN(C(=O)C2CC2)C1] | 474.4 | 0.16 |
| 80 | 1-(1-(1,4-dioxane-2- carbonyl)pyrrolidin-3-yl)-4-chloro-N- (3-methyl-5-(phenylethynyl)pyridin-2- yl)-1H-pyrazole-5-carboxamide | 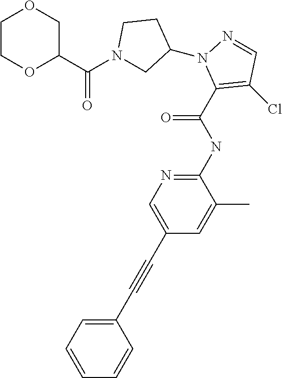 [SMILES: Cc1cc(C#Cc2ccccc2)cnc1NC(=O)c1c(Cl)cnn1C1CCN(C(=O)C2COCCO2)C1] | 520.3 | 0.16 |
| 81 | 1-(1-(1,4-dioxane-2-carbonyl)azetidin- 3-yl)-4-chloro-N-(3-methyl-5- (phenylethynyl)pyridin-2-yl)-1H- pyrazole-5-carboxamide | 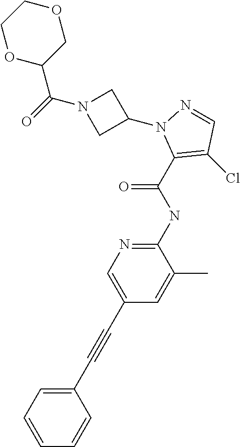 [SMILES: Cc1cc(C#Cc2ccccc2)cnc1NC(=O)c1c(Cl)cnn1C1CN(C(=O)C2COCCO2)C1] | 506.2 | 0.16 |
| 82 | 1-(((3S,4R)-1-acetyl-3- fluoropiperidin-4-yl)methyl)-4-chloro- N-(3-methyl-5- (phenylethynyl)pyridin-2-yl)-1H- pyrazole-5-carboxamide | 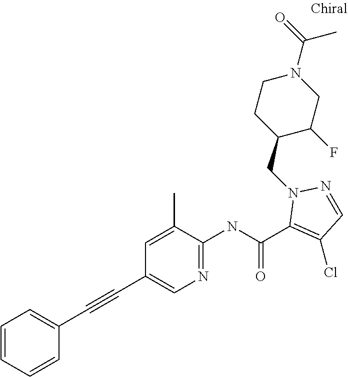 [SMILES: CC(=O)N1CC[C@H](Cn2ncc(Cl)c2C(=O)Nc2ncc(C#Cc3ccccc3)cc2C)C(F)C1] | 494.2 | 0.16 |
| 83 | tert-butyl 4-((4-chloro-5-((3-fluoro-5- (phenylethynyl)pyridin-2- yl)carbamoyl)-1H-pyrazol-1- yl)methyl)-4-cyanopiperidine-1- carboxylate | 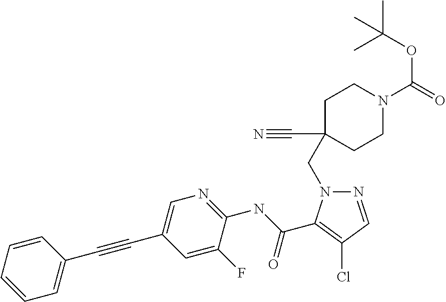 [SMILES: CC(C)(C)OC(=O)N1CCC(C#N)(Cn2ncc(Cl)c2C(=O)Nc2ncc(C#Cc3ccccc3)cc2F)CC1] | 563.2 | 0.16 |
| 84 | 1-((1-acetyl-4-cyanopiperidin-4- yl)methyl)-4-chloro-N-(3-methyl-5- (phenylethynyl)pyridin-2-yl)-1H- pyrazole-5-carboxamide | 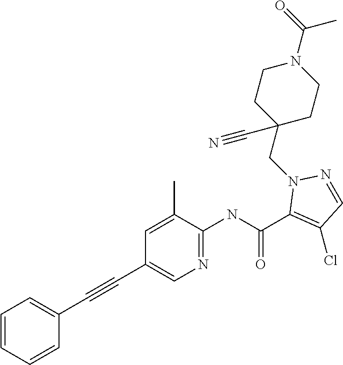 [SMILES: CC(=O)N1CCC(C#N)(Cn2ncc(Cl)c2C(=O)Nc2ncc(C#Cc3ccccc3)cc2C)CC1] | 501.4 | 0.16 |
| 85 | 1-((1-acetylpiperidin-4-yl)methyl)-4- chloro-N-(3-methyl-5- (phenylethynyl)pyridin-2-yl)-1H- pyrazole-5-carboxamide | 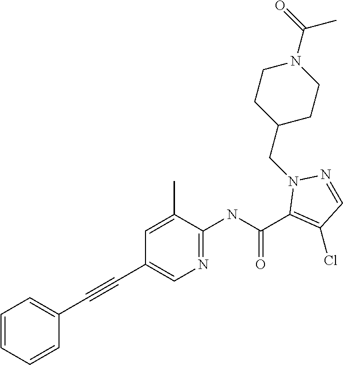 [SMILES: CC(=O)N1CCC(Cn2ncc(Cl)c2C(=O)Nc2ncc(C#Cc3ccccc3)cc2C)CC1] | 476.4 | 0.17 |
| 86*⁷ | 1-[(2S,4R)-1-acetyl-2- methylpiperidin-4-yl]-4-chloro-N-[3- methyl-5-(phenylethynyl)pyridin-2- yl]-1H-pyrazole-5-carboxamide or 1-[(2R,4S)-1-acetyl-2- methylpiperidin-4-yl]-4-chloro-N-[3- methyl-5-(phenylethynyl)pyridin-2- yl]-1H-pyrazole-5-carboxamide | 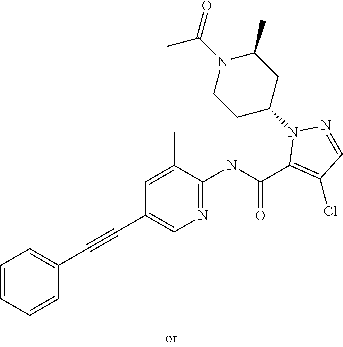 [SMILES: CC(=O)N1CC[C@@H](n2ncc(Cl)c2C(=O)Nc2ncc(C#Cc3ccccc3)cc2C)C[C@@H]1C] | 476.4 | 0.17 |
| | | 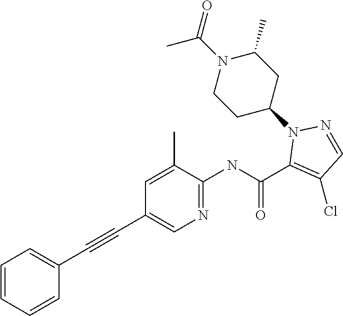 [SMILES: CC(=O)N1CC[C@H](n2ncc(Cl)c2C(=O)Nc2ncc(C#Cc3ccccc3)cc2C)C[C@H]1C] | | |
| 87 | 4-chloro-1-(1- (cyclopropanecarbonyl)azetidin-3-yl)- N-(3-methyl-5- (phenylethynyl)pyridin-2-yl)-1H- pyrazole-5-carboxamide | 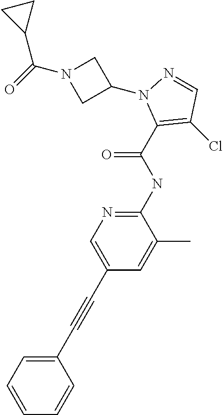 [SMILES: Cc1cc(C#Cc2ccccc2)cnc1NC(=O)c1c(Cl)cnn1C1CN(C(=O)C2CC2)C1] | 460.4 | 0.17 |
| 88 | 1-(((3R,4S)-1-acetyl-3- fluoropiperidin-4-yl)methyl)-4-chloro- N-(3-fluoro-5-(phenylethynyl)pyridin- 2-yl)-1H-pyrazole-5-carboxamide | 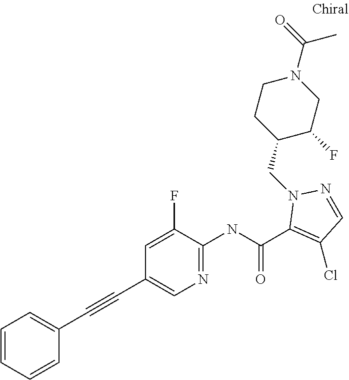 [SMILES: CC(=O)N1CC[C@@H](Cn2ncc(Cl)c2C(=O)Nc2ncc(C#Cc3ccccc3)cc2F)[C@@H](F)C1] | 498.3 | 0.17 |
| 89 | (S)-4-chloro-N-(3-methyl-5-(thiophen- 2-ylethynyl)pyridin-2-yl)-1- (tetrahydrofuran-3-yl)-1H-pyrazole-5- carboxamide | 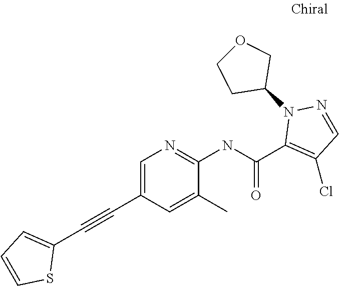 [SMILES: Cc1cc(C#Cc2cccs2)cnc1NC(=O)c1c(Cl)cnn1[C@H]1CCOC1] | 413.3 | 0.18 |
| 90 | 4-(4-chloro-5-((3-methyl-5- (phenylethynyl)pyridin-2- yl)carbamoyl)-1H-pyrazol-1-yl)-N- ethylpiperidine-1-carboxamide | 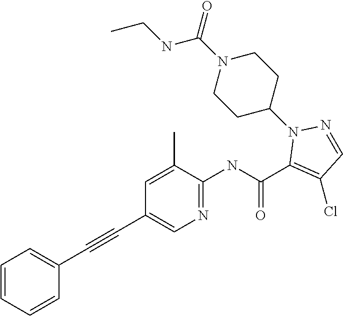 [SMILES: CCNC(=O)N1CCC(n2ncc(Cl)c2C(=O)Nc2ncc(C#Cc3ccccc3)cc2C)CC1] | 491.3 | 0.18 |
| 91 | 4-chloro-N-(3-fluoro-5- (phenylethynyl)pyridin-2-yl)-1-(1-(2- methylcyclopropane-1- carbonyl)piperidin-4-yl)-1H-pyrazole- 5-carboxamide | 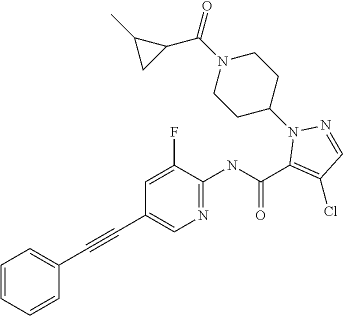 [SMILES: CC1CC1C(=O)N1CCC(n2ncc(Cl)c2C(=O)Nc2ncc(C#Cc3ccccc3)cc2F)CC1] | 506.3 | 0.18 |
| 92 | 4-(4-chloro-5-((5-((4- fluorophenyl)ethynyl)-3- methylpyridin-2-yl)carbamoyl)-1H- pyrazol-1-yl)-N-ethylpiperidine-1- carboxamide | 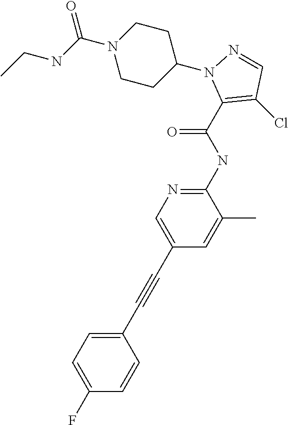 [SMILES: CCNC(=O)N1CCC(n2ncc(Cl)c2C(=O)Nc2ncc(C#Cc3ccc(F)cc3)cc2C)CC1] | 509.4 | 0.18 |
| 93 | 1-((1-acetylazetidin-3-yl)methyl)-4- chloro-N-(3-fluoro-5- (phenylethynyl)pyridin-2-yl)-1H- pyrazole-5-carboxamide | 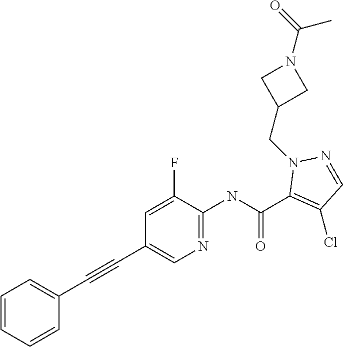 [SMILES: CC(=O)N1CC(Cn2ncc(Cl)c2C(=O)Nc2ncc(C#Cc3ccccc3)cc2F)C1] | 452 | 0.18 |
| 94 | 1-(cis-3-acetamidocyclobutyl)-4- chloro-N-(3-fluoro-5- (phenylethynyl)pyridin-2-yl)-1H- pyrazole-5-carboxamide | 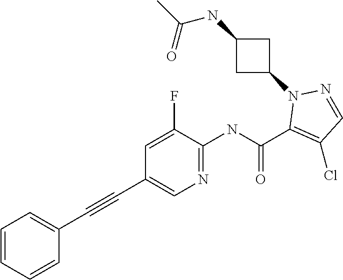 [SMILES: CC(=O)N[C@H]1C[C@@H](n2ncc(Cl)c2C(=O)Nc2ncc(C#Cc3ccccc3)cc2F)C1] | 452.2 | 0.18 |
| 95 | 1-(1-(1,4-dioxane-2- carbonyl)piperidin-4-yl)-4-chloro-N- (3-fluoro-5-(thiophen-2- ylethynyl)pyridin-2-yl)-1H-pyrazole- 5-carboxamide | 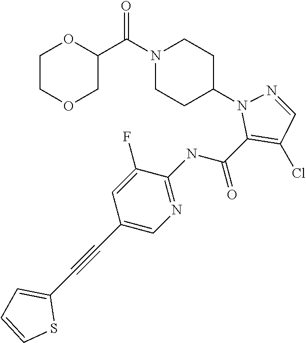 [SMILES: O=C(Nc1ncc(C#Cc2cccs2)cc1F)c1c(Cl)cnn1C1CCN(C(=O)C2COCCO2)CC1] | 544 | 0.18 |
| 96 | 4-chloro-1-(2-(4- (cyclopropanecarbonyl)piperazin-1- yl)ethyl)-N-(5-((4- fluorophenyl)ethynyl)-3- methylpyridin-2-yl)-1H-pyrazole-5- carboxamide | 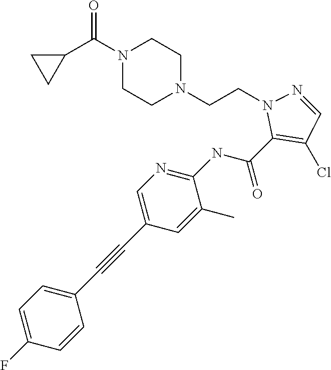 [SMILES: Cc1cc(C#Cc2ccc(F)cc2)cnc1NC(=O)c1c(Cl)cnn1CCN1CCN(C(=O)C2CC2)CC1] | 535.3 | 0.18 |
| 97 | 1-(2-(1,4-dioxane-2-carbonyl)-2- azaspiro[3.3]heptan-6-yl)-4-chloro-N- (3-methyl-5-(thiophen-2- ylethynyl)pyridin-2-yl)-1H-pyrazole- 5-carboxamide | 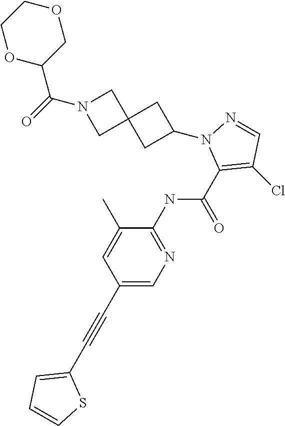 [SMILES: Cc1cc(C#Cc2cccs2)cnc1NC(=O)c1c(Cl)cnn1C1CC2(C1)CN(C(=O)C1COCCO1)C2] | 552.3 | 0.18 |
| 98 | 1-((1-acetyl-3,3-difluoropiperidin-4- yl)methyl)-4-chloro-N-(3-methyl-5- (phenylethynyl)pyridin-2-yl)-1H- pyrazole-5-carboxamide | 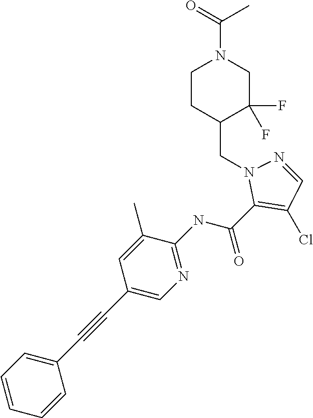 [SMILES: CC(=O)N1CCC(Cn2ncc(Cl)c2C(=O)Nc2ncc(C#Cc3ccccc3)cc2C)C(F)(F)C1] | 512.2 | 0.18 |
| 99 | 1-((3R,4S)-1-acetyl-3-fluoropiperidin- 4-yl)-4-chloro-N-(3-methyl-5- (phenylethynyl)pyridin-2-yl)-1H- pyrazole-5-carboxamide | 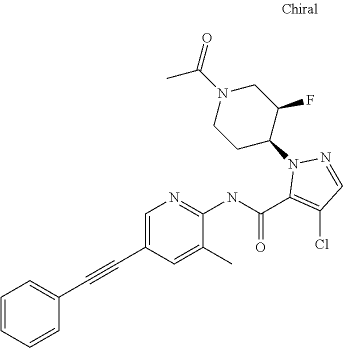 [SMILES: CC(=O)N1CC[C@H](n2ncc(Cl)c2C(=O)Nc2ncc(C#Cc3ccccc3)cc2C)[C@H](F)C1] | 480.3 | 0.18 |
| 100 | 4-chloro-N-(3-fluoro-5- (phenylethynyl)pyridin-2-yl)-1-(1- pivaloylpiperidin-4-yl)-1H-pyrazole-5- carboxamide | 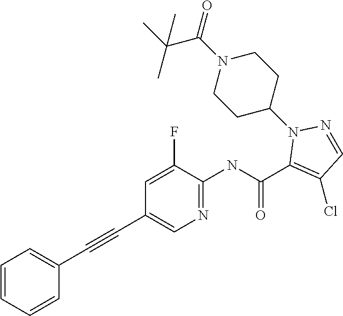 [SMILES: CC(C)(C)C(=O)N1CCC(n2ncc(Cl)c2C(=O)Nc2ncc(C#Cc3ccccc3)cc2F)CC1] | 508.3 | 0.19 |
| 101 | 4-chloro-1-(1- (cyclopropanecarbonyl)piperidin-4- yl)-N-(5-((4-fluorophenyl)ethynyl)-3- methylpyridin-2-yl)-1H-pyrazole-5- carboxamide | 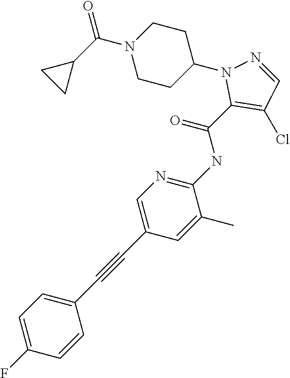 [SMILES: Cc1cc(C#Cc2ccc(F)cc2)cnc1NC(=O)c1c(Cl)cnn1C1CCN(C(=O)C2CC2)CC1] | 506.3 | 0.19 |
| 102 | 1-(2-(1-acetylpiperidin-4-yl)ethyl)-4- chloro-N-(3-fluoro-5- (phenylethynyl)pyridin-2-yl)-1H- pyrazole-5-carboxamide | 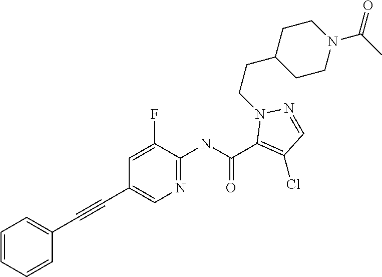 [SMILES: CC(=O)N1CCC(CCn2ncc(Cl)c2C(=O)Nc2ncc(C#Cc3ccccc3)cc2F)CC1] | 494.3 | 0.19 |
| 103 | 1-((1-(1,4-dioxane-2- carbonyl)piperidin-4-yl)methyl)-4- chloro-N-(3-methyl-5-(thiophen-2- ylethynyl)pyridin-2-yl)-1H-pyrazole- 5-carboxamide | 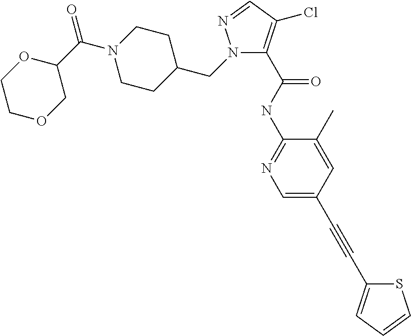 [SMILES: Cc1cc(C#Cc2cccs2)cnc1NC(=O)c1c(Cl)cnn1CC1CCN(C(=O)C2COCCO2)CC1] | 554.3 | 0.19 |
| 104 | 1-((1-(1,4-dioxane-2- carbonyl)piperidin-4-yl)methyl)-4- chloro-N-(5-((3- fluorophenyl)ethynyl)-3- methylpyridin-2-yl)-1H-pyrazole-5- carboxamide | 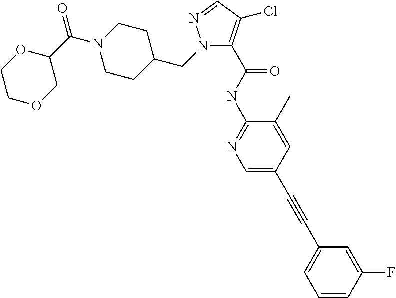 [SMILES: Cc1cc(C#Cc2cccc(F)c2)cnc1NC(=O)c1c(Cl)cnn1CC1CCN(C(=O)C2COCCO2)CC1] | 566.2 | 0.19 |
| 105 | 1-((4-acetylmorpholin-2-yl)methyl)-4- chloro-N-(3-methyl-5- (phenylethynyl)pyridin-2-yl)-1H- pyrazole-5-carboxamide | 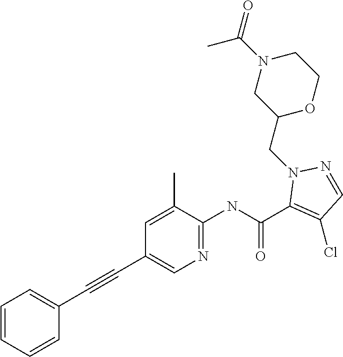 [SMILES: CC(=O)N1CCOC(Cn2ncc(Cl)c2C(=O)Nc2ncc(C#Cc3ccccc3)cc2C)C1] | 478.3 | 0.19 |
| 106 | 4-chloro-N-(3-methyl-5- (phenylethynyl)pyridin-2-yl)-1-(1- propionylpyrrolidin-3-yl)-1H- pyrazole-5-carboxamide | 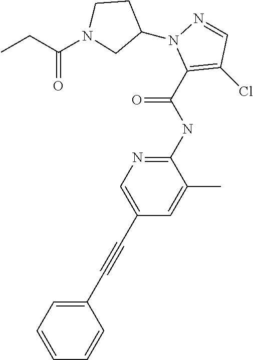 [SMILES: CCC(=O)N1CCC(n2ncc(Cl)c2C(=O)Nc2ncc(C#Cc3ccccc3)cc2C)C1] | 462.4 | 0.19 |
| 107 | 1-(3-acetamidocyclohexyl)-4-chloro- N-(3-methyl-5- (phenylethynyl)pyridin-2-yl)-1H- pyrazole-5-carboxamide hydrofluoride | 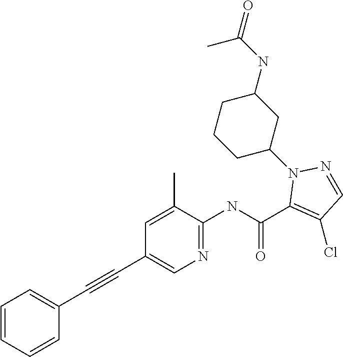 [SMILES: CC(=O)NC1CCCC(n2ncc(Cl)c2C(=O)Nc2ncc(C#Cc3ccccc3)cc2C)C1] | 476.3 | 0.19 |
| 108 | 4-chloro-1-(((3S,4R)-3-fluoro-1- isobutyrylpiperidin-4-yl)methyl)-N-(3- methyl-5-(phenylethynyl)pyridin-2- yl)-1H-pyrazole-5-carboxamide | 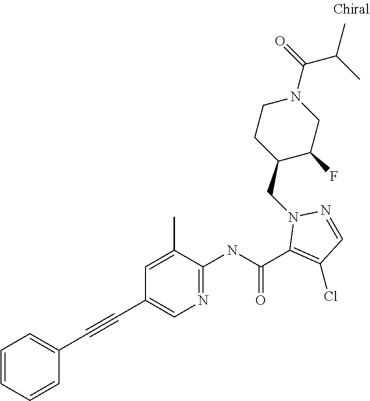 [SMILES: Cc1cc(C#Cc2ccccc2)cnc1NC(=O)c1c(Cl)cnn1C[C@H]1CCN(C(=O)C(C)C)C[C@H]1F] | 522.4 | 0.19 |
| 109 | 1-(((3R,4S)-1-acetyl-3- fluoropiperidin-4-yl)methyl)-4-chloro- N-(3-methyl-5- (phenylethynyl)pyridin-2-yl)-1H- pyrazole-5-carboxamide | 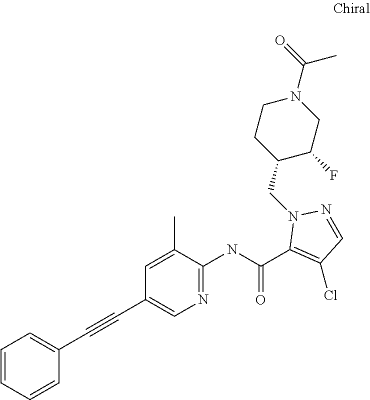 [SMILES: CC(=O)N1CC[C@@H](Cn2ncc(Cl)c2C(=O)Nc2ncc(C#Cc3ccccc3)cc2C)[C@@H](F)C1] | 494.2 | 0.19 |
| 110 | 1-[(3aR,5s,6aS)-2- acetyloctahydrocyclopenta[c]pyrrol-5- yl]-4-chloro-N-{3-methyl-5- [(thiophen-2-yl)ethynyl]pyridin-2-yl}- 1H-pyrazole-5-carboxamide | 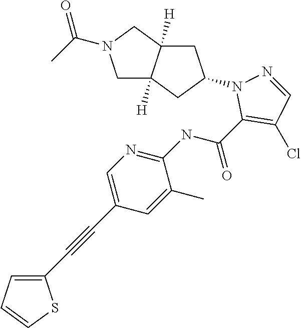 [SMILES: CC(=O)N1C[C@H]2C[C@H](n3ncc(Cl)c3C(=O)Nc3ncc(C#Cc4cccs4)cc3C)C[C@H]2C1] | 495.3 | 0.2 |
| 111 | 4-chloro-1-(1-isobutyrylpiperidin-4- yl)-N-(3-methyl-5-(thiophen-2- ylethynyl)pyridin-2-yl)-1H-pyrazole- 5-carboxamide | 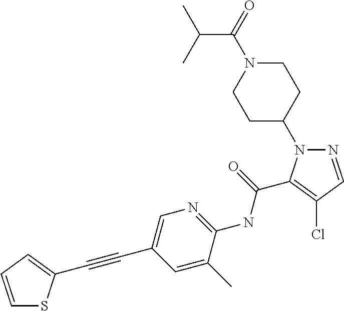 [SMILES: Cc1cc(C#Cc2cccs2)cnc1NC(=O)c1c(Cl)cnn1C1CCN(C(=O)C(C)C)CC1] | 496.4 | 0.2 |
| 112 | 4-chloro-N-(3-fluoro-5- (phenylethynyl)pyridin-2-yl)-1-(1-(1- methylcyclobutane-1- carbonyl)piperidin-4-yl)-1H-pyrazole- 5-carboxamide | 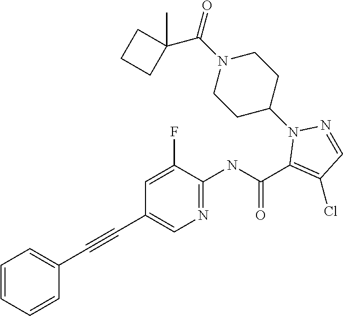 [SMILES: CC1(C(=O)N2CCC(n3ncc(Cl)c3C(=O)Nc3ncc(C#Cc4ccccc4)cc3F)CC2)CCC1] | 520.3 | 0.2 |
| 113 | 1-(2-acetyl-2-azaspiro[3.3]heptan-6- yl)-4-chloro-N-(3-fluoro-5- (phenylethynyl)pyridin-2-yl)-1H- pyrazole-5-carboxamide | 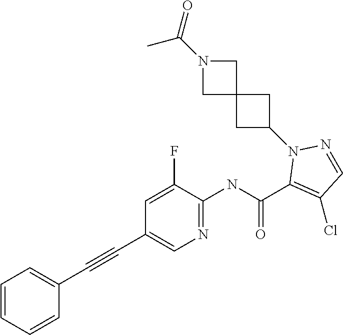 [SMILES: CC(=O)N1CC2(CC(n3ncc(Cl)c3C(=O)Nc3ncc(C#Cc4ccccc4)cc3F)C2)C1] | 478.3 | 0.2 |
| 114*⁴ | 1-[(2S,4S)-1-acetyl-2-methylpiperidin- 4-yl]-4-chloro-N-[3-methyl-5- (phenylethynyl)pyridin-2-yl]-1H- pyrazole-5-carboxamide or 1-[(2R,4R)-1-acetyl-2- methylpiperidin-4-yl]-4-chloro-N-[3- methyl-5-(phenylethynyl)pyridin-2- yl]-1H-pyrazole-5-carboxamide | 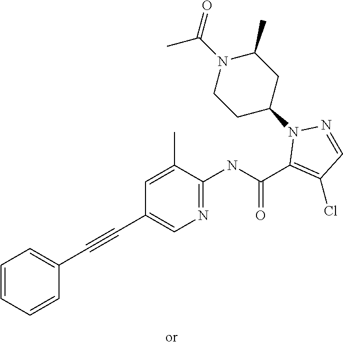 [SMILES: CC(=O)N1CC[C@H](n2ncc(Cl)c2C(=O)Nc2ncc(C#Cc3ccccc3)cc2C)C[C@@H]1C] | 476.3 | 0.2 |
| | | 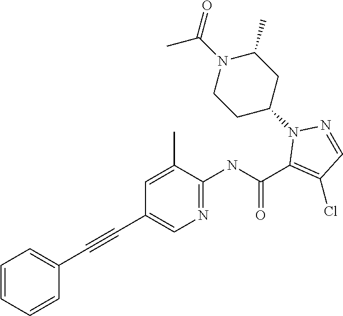 [SMILES: CC(=O)N1CC[C@@H](n2ncc(Cl)c2C(=O)Nc2ncc(C#Cc3ccccc3)cc2C)C[C@H]1C] | | |
| 115 | 4-chloro-1-(2-(cyclopropanecarbonyl)- 2-azaspiro[3.3]heptan-6-yl)-N-(5-((4- fluorophenyl)ethynyl)-3- methylpyridin-2-yl)-1H-pyrazole-5- carboxamide | 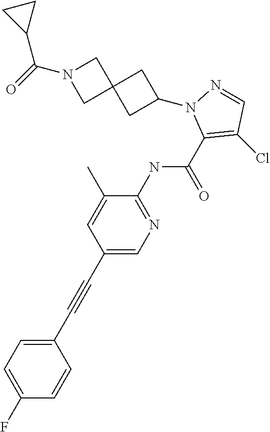 [SMILES: Cc1cc(C#Cc2ccc(F)cc2)cnc1NC(=O)c1c(Cl)cnn1C1CC2(C1)CN(C(=O)C1CC1)C2] | 518.4 | 0.2 |
| 116 | 4-chloro-N-(5-((4- fluorophenyl)ethynyl)-3- methylpyridin-2-yl)-1-(2-isobutyryl-2- azaspiro[3.3]heptan-6-yl)-1H- pyrazole-5-carboxamide | 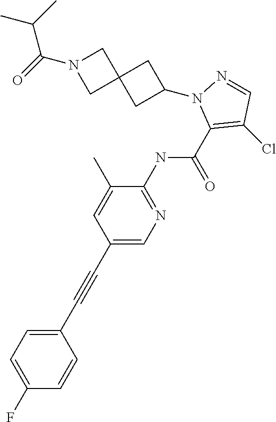 [SMILES: Cc1cc(C#Cc2ccc(F)cc2)cnc1NC(=O)c1c(Cl)cnn1C1CC2(C1)CN(C(=O)C(C)C)C2] | 520.4 | 0.2 |
| 117 | tert-butyl (trans-4-(4-chloro-5-((3- fluoro-5-((4- fluorophenyl)ethynyl)pyridin-2- yl)carbamoyl)-1H-pyrazol-1- yl)cyclohexyl)carbamate | 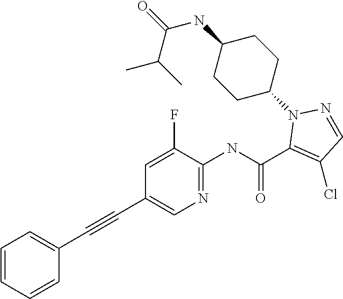 [SMILES: CC(C)C(=O)N[C@H]1CC[C@H](n2ncc(Cl)c2C(=O)Nc2ncc(C#Cc3ccccc3)cc2F)CC1] | 508.3 | 0.2 |
| 118 | 4-chloro-1-(((3R,4S)-3-fluoro-1- isobutyrylpiperidin-4-yl)methyl)-N-(3- methyl-5-(phenylethynyl)pyridin-2- yl)-1H-pyrazole-5-carboxamide | 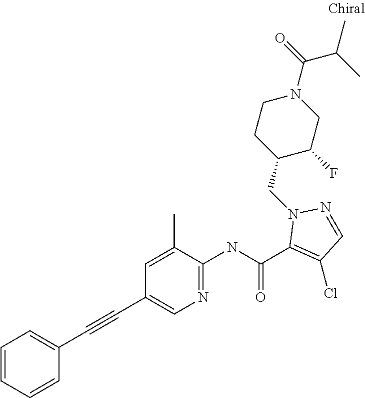 [SMILES: Cc1cc(C#Cc2ccccc2)cnc1NC(=O)c1c(Cl)cnn1C[C@@H]1CCN(C(=O)C(C)C)C[C@@H]1F] | 522.2 | 0.2 |
| 119 | 4-chloro-1-((4-cyano-1- isobutyrylpiperidin-4-yl)methyl)-N-(5- ((4-fluorophenyl)ethynyl)-3- methylpyridin-2-yl)-1H-pyrazole-5- carboxamide | 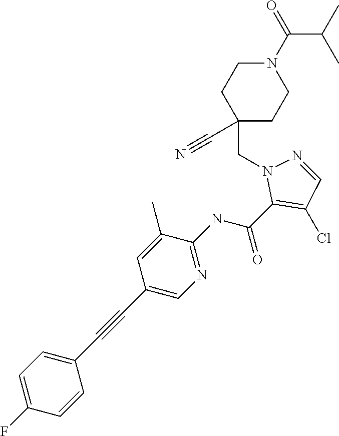 [SMILES: Cc1cc(C#Cc2ccc(F)cc2)cnc1NC(=O)c1c(Cl)cnn1CC1(C#N)CCN(C(=O)C(C)C)CC1] | 547.3 | 0.2 |
| 120 | 4-chloro-1-((1-isobutyryl-3- methylpiperidin-4-yl)methyl)-N-(3- methyl-5-(phenylethynyl)pyridin-2- yl)-1H-pyrazole-5-carboxamide hydrofluoride | 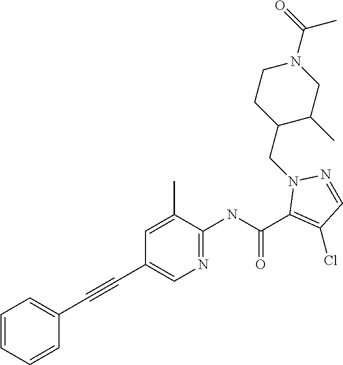 [SMILES: CC(=O)N1CCC(Cn2ncc(Cl)c2C(=O)Nc2ncc(C#Cc3ccccc3)cc2C)C(C)C1] | 490.4 | 0.2 |
| 121 | 1-(1-acetylpyrrolidin-3-yl)-4-chloro- N-(3-fluoro-5-(phenylethynyl)pyridin- 2-yl)-1H-pyrazole-5-carboxamide | 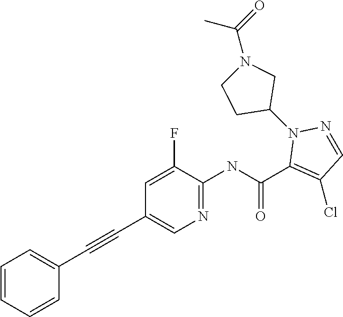 [SMILES: CC(=O)N1CCC(n2ncc(Cl)c2C(=O)Nc2ncc(C#Cc3ccccc3)cc2F)C1] | 452 | 0.21 |
| 122 | 1-(1-acetylazetidin-3-yl)-4-chloro-N- (3-methyl-5-(phenylethynyl)pyridin-2- yl)-1H-pyrazole-5-carboxamide | 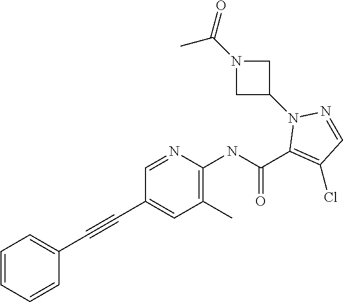 [SMILES: CC(=O)N1CC(n2ncc(Cl)c2C(=O)Nc2ncc(C#Cc3ccccc3)cc2C)C1] | 434 | 0.21 |
| 123 | 1-((1-acetylpiperidin-4-yl)methyl)-4- chloro-N-(3-methyl-5-(thiophen-2- ylethynyl)pyridin-2-yl)-1H-pyrazole-5- carboxamide | 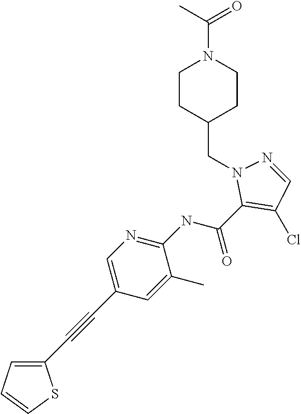 [SMILES: CC(=O)N1CCC(Cn2ncc(Cl)c2C(=O)Nc2ncc(C#Cc3cccs3)cc2C)CC1] | 482.3 | 0.21 |
| 124 | 4-chloro-N-(3-fluoro-5- (phenylethynyl)pyridin-2-yl)-1-(cis-3- isobutyramidocyclobutyl)-1H- pyrazole-5-carboxamide | 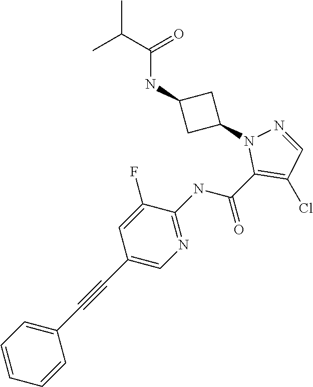 [SMILES: CC(C)C(=O)N[C@H]1C[C@@H](n2ncc(Cl)c2C(=O)Nc2ncc(C#Cc3ccccc3)cc2F)C1] | 480.3 | 0.21 |
| 125 | 4-chloro-N-(5-((4- fluorophenyl)ethynyl)-3- methylpyridin-2-yl)-1-(cis-4- isobutyramidocyclohexyl)-1H- pyrazole-5-carboxamide | 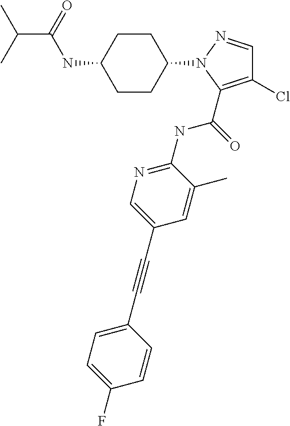 [SMILES: Cc1cc(C#Cc2ccc(F)cc2)cnc1NC(=O)c1c(Cl)cnn1[C@H]1CC[C@@H](NC(=O)C(C)C)CC1] | 522.4 | 0.21 |
| 126 | tert-butyl 4-((4-chloro-5-((3-methyl-5- (phenylethynyl)pyridin-2- yl)carbamoyl)-1H-pyrazol-1- yl)methyl)-4-cyanopiperidine-1- carboxylate | 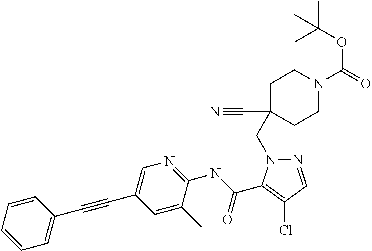 [SMILES: Cc1cc(C#Cc2ccccc2)cnc1NC(=O)c1c(Cl)cnn1CC1(C#N)CCN(C(=O)OC(C)(C)C)CC1] | 559.3 | 0.21 |
| 127 | 4-chloro-1-((1- cyanocyclopropyl)methyl)-N-(2- fluoro-6-methyl-4- (phenylethynyl)phenyl)-1H-pyrazole- 5-carboxamide | 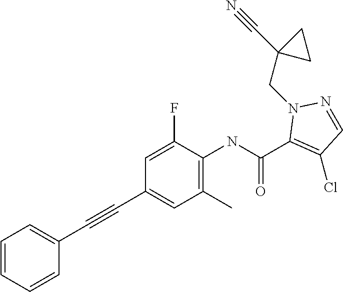 [SMILES: Cc1cc(C#Cc2ccccc2)cc(F)c1NC(=O)c1c(Cl)cnn1CC1(C#N)CC1] | 433 | 0.22 |
| 128 | (S)-1-(1-acetylpyrrolidin-3-yl)-4- chloro-N-(3-methyl-5- (phenylethynyl)pyridin-2-yl)-1H- pyrazole-5-carboxamide | 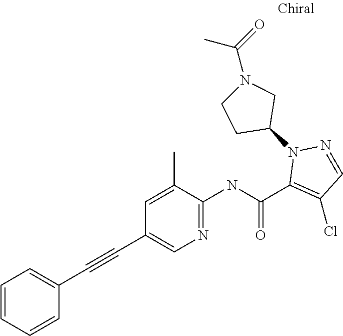 [SMILES: CC(=O)N1CC[C@H](n2ncc(Cl)c2C(=O)Nc2ncc(C#Cc3ccccc3)cc2C)C1] | 448 | 0.22 |
| 129 | (S)-1-(1-acetylpyrrolidin-3-yl)-4- chloro-N-(3-fluoro-5- (phenylethynyl)pyridin-2-yl)-1H- pyrazole-5-carboxamide | 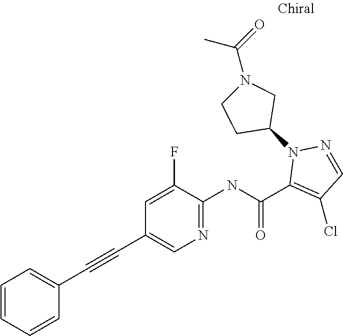 [SMILES: CC(=O)N1CC[C@H](n2ncc(Cl)c2C(=O)Nc2ncc(C#Cc3ccccc3)cc2F)C1] | 452 | 0.22 |
| 130 | 4-chloro-1-(1- (cyclopropanecarbonyl)piperidin-4- yl)-N-(3-fluoro-5- (phenylethynyl)pyridin-2-yl)-1H- pyrazole-5-carboxamide | 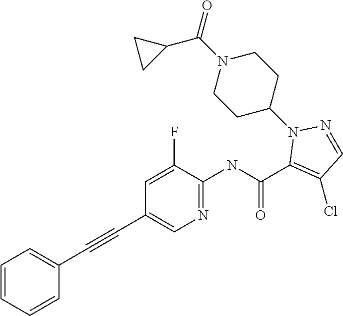 [SMILES: O=C(Nc1ncc(C#Cc2ccccc2)cc1F)c1c(Cl)cnn1C1CCN(C(=O)C2CC2)CC1] | 492.3 | 0.22 |
| 131 | 1-(1-acetylazepan-4-yl)-4-chloro-N- (3-methyl-5-(phenylethynyl)pyridin-2- yl)-1H-pyrazole-5-carboxamide | 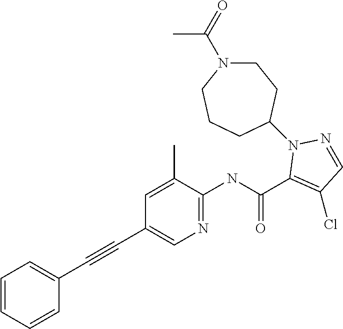 [SMILES: CC(=O)N1CCCC(n2ncc(Cl)c2C(=O)Nc2ncc(C#Cc3ccccc3)cc2C)CC1] | 476.3 | 0.22 |
| 132 | (3aR,5s,6aS)-5-[4-chloro-5-({5-[(3- fluorophenyl)ethynyl]-3- methylpyridin-2-yl}carbamoyl)-1H- pyrazol-1-yl]-N- ethylhexahydrocyclopenta[c]pyrrole- 2(1H)-carboxamide | 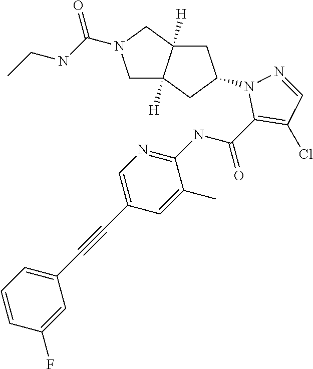 [SMILES: CCNC(=O)N1C[C@H]2C[C@H](n3ncc(Cl)c3C(=O)Nc3ncc(C#Cc4cccc(F)c4)cc3C)C[C@H]2C1] | 535.3 | 0.22 |
| 133 | 1-(2-(1-acetylpiperidin-4-yl)ethyl)-4- chloro-N-(5-((3- fluorophenyl)ethynyl)-3- methylpyridin-2-yl)-1H-pyrazole-5- carboxamide | 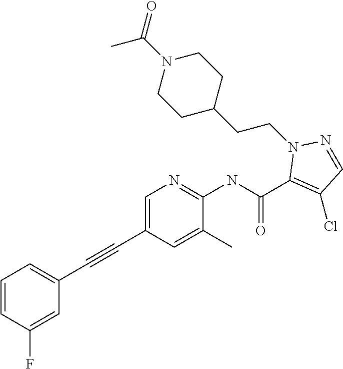 [SMILES: CC(=O)N1CCC(CCn2ncc(Cl)c2C(=O)Nc2ncc(C#Cc3cccc(F)c3)cc2C)CC1] | 508 | 0.22 |
| 134 | tert-butyl 4-((4-chloro-5-((3-fluoro-5- (phenylethynyl)pyridin-2- yl)carbamoyl)-1H-pyrazol-1- yl)methyl)-4-fluoropiperidine-1- carboxylate | 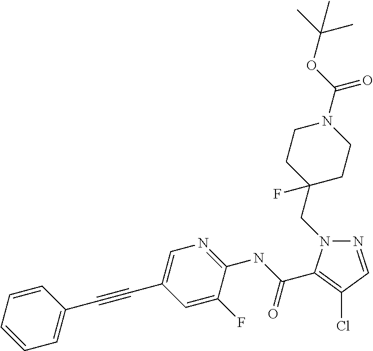 [SMILES: CC(C)(C)OC(=O)N1CCC(F)(Cn2ncc(Cl)c2C(=O)Nc2ncc(C#Cc3ccccc3)cc2F)CC1] | 556.3 | 0.22 |
| 135 | 1-((1-acetylazetidin-3-yl)methyl)-4- chloro-N-(3-methyl-5- (phenylethynyl)pyridin-2-yl)-1H- pyrazole-5-carboxamide | 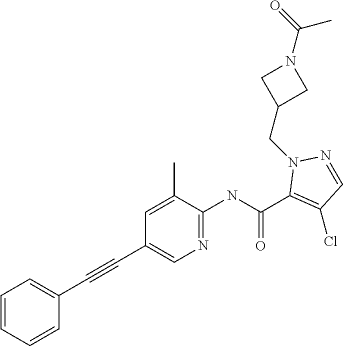 [SMILES: CC(=O)N1CC(Cn2ncc(Cl)c2C(=O)Nc2ncc(C#Cc3ccccc3)cc2C)C1] | 448.2 | 0.22 |
| 136 | 4-chloro-1-(1-isobutyrylpyrrolidin-3- yl)-N-(3-methyl-5- (phenylethynyl)pyridin-2-yl)-1H- pyrazole-5-carboxamide | 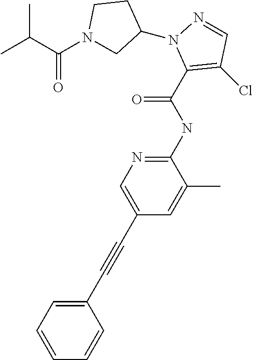 [SMILES: Cc1cc(C#Cc2ccccc2)cnc1NC(=O)c1c(Cl)cnn1C1CCN(C(=O)C(C)C)C1] | 476.4 | 0.22 |
| 137 | 4-chloro-N-(3-methyl-5- (phenylethynyl)pyridin-2-yl)-1-(1- propionylazetidin-3-yl)-1H-pyrazole- 5-carboxamide | 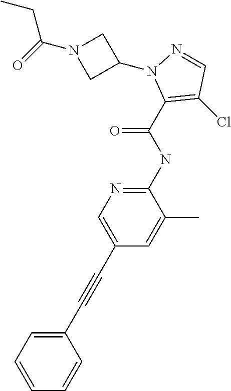 [SMILES: CCC(=O)N1CC(n2ncc(Cl)c2C(=O)Nc2ncc(C#Cc3ccccc3)cc2C)C1] | 448.2 | 0.22 |
| 138 | 1-(2-(1,4-dioxane-2-carbonyl)-2- azaspiro[3.3]heptan-6-yl)-4-chloro-N- (5-((4-fluorophenyl)ethynyl)-3- methylpyridin-2-yl)-1H-pyrazole-5- carboxamide | 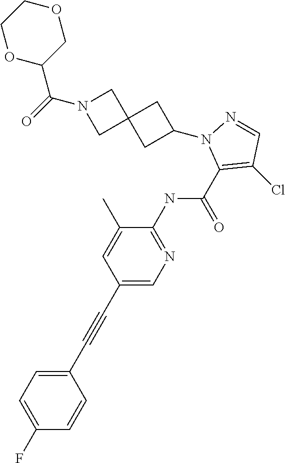 [SMILES: Cc1cc(C#Cc2ccc(F)cc2)cnc1NC(=O)c1c(Cl)cnn1C1CC2(C1)CN(C(=O)C1COCCO1)C2] | 564.4 | 0.22 |
| 139 | 4-chloro-1-(1- (cyclopropanecarbonyl)piperidin-4- yl)-N-(3-fluoro-5-(thiophen-2- ylethynyl)pyridin-2-yl)-1H-pyrazole- 5-carboxamide | 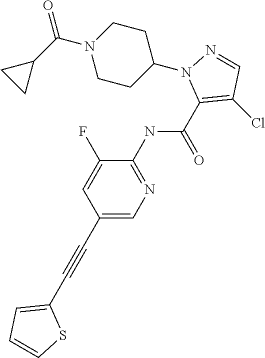 [SMILES: O=C(Nc1ncc(C#Cc2cccs2)cc1F)c1c(Cl)cnn1C1CCN(C(=O)C2CC2)CC1] | 498.2 | 0.22 |
| 140 | 1-((1-acetyl-3-fluoroazetidin-3- yl)methyl)-4-chloro-N-(3-fluoro-5- (phenyethynyl)pyridin-2-yl)-1H- pyrazole-5-carboxamide | 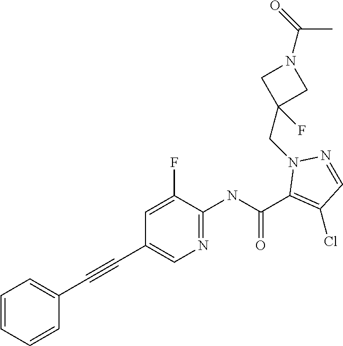 [SMILES: CC(=O)N1CC(F)(Cn2ncc(Cl)c2C(=O)Nc2ncc(C#Cc3ccccc3)cc2F)C1] | 470.3 | 0.22 |
| 141 | 4-chloro-1-((1- cyanocyclopropyl)methyl)-N-(3- fluoro-5-(phenylethynyl)pyridin-2-yl)- 1H-pyrazole-5-carboxamide | 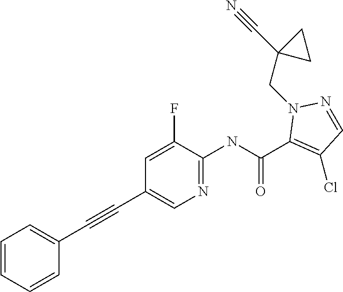 [SMILES: N#CC1(Cn2ncc(Cl)c2C(=O)Nc2ncc(C#Cc3ccccc3)cc2F)CC1] | 420 | 0.23 |
| 142 | tert-butyl 4-((4-chloro-5-((3-fluoro-5- (phenylethynyl)pyridin-2- yl)carbamoyl)-1H-pyrazol-1- yl)methyl)piperidine-1-carboxylate | 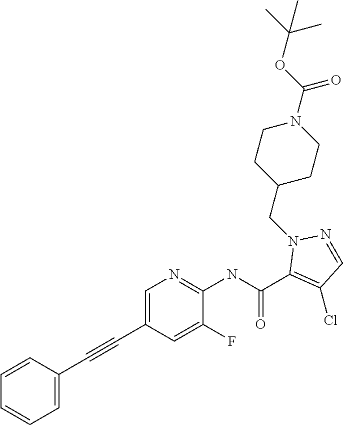 [SMILES: CC(C)(C)OC(=O)N1CCC(Cn2ncc(Cl)c2C(=O)Nc2ncc(C#Cc3ccccc3)cc2F)CC1] | 538.3 | 0.23 |
| 143 | 4-chloro-1-(1- (cyclopropanecarbonyl)piperidin-4- yl)-N-(5-((3-fluorophenyl)ethynyl)-3- methylpyridin-2-yl)-1H-pyrazole-5- carboxamide | 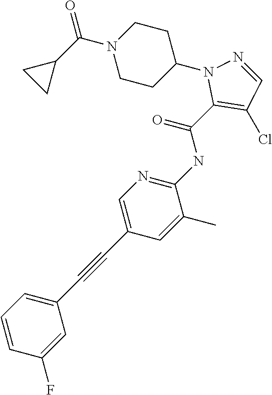 [SMILES: Cc1cc(C#Cc2cccc(F)c2)cnc1NC(=O)c1c(Cl)cnn1C1CCN(C(=O)C2CC2)CC1] | 506.3 | 0.23 |
| 144 | 4-chloro-N-(3-fluoro-5- (phenylethynyl)pyridin-2-yl)-1-(2-(1- isobutyrylpiperidin-4-yl)ethyl)-1H- pyrazole-5-carboxamide | 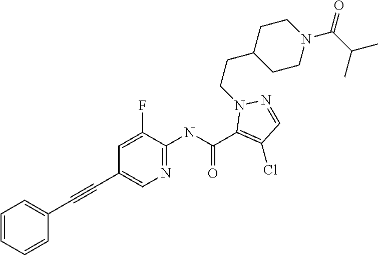 [SMILES: CC(C)C(=O)N1CCC(CCn2ncc(Cl)c2C(=O)Nc2ncc(C#Cc3ccccc3)cc2F)CC1] | 522.4 | 0.23 |
| 145 | 4-chloro-N-(3-fluoro-5- (phenylethynyl)pyridin-2-yl)-1-((4- isobutyrylmorpholin-2-yl)methyl)-1H- pyrazole-5-carboxamide | 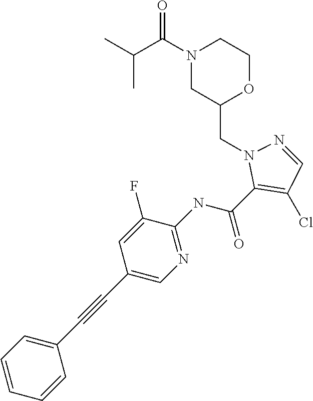 [SMILES: CC(C)C(=O)N1CCOC(Cn2ncc(Cl)c2C(=O)Nc2ncc(C#Cc3ccccc3)cc2F)C1] | 510.2 | 0.23 |
| 146 | 4-chloro-1-(((3R,4S)-3-fluoro-1- isobutyrylpiperidin-4-yl)methyl)-N-(3- fluoro-5-(phenylethynyl)pyridin-2-yl)- 1H-pyrazole-5-carboxamide | 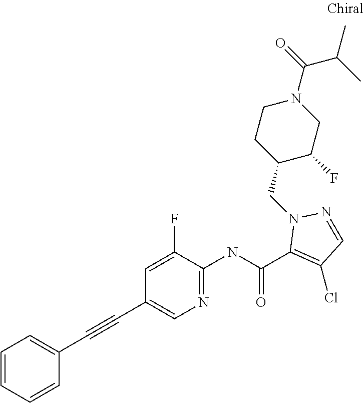 [SMILES: CC(C)C(=O)N1CC[C@@H](Cn2ncc(Cl)c2C(=O)Nc2ncc(C#Cc3ccccc3)cc2F)[C@@H](F)C1] | 526.3 | 0.23 |
| 147 | (R)-4-chloro-N-(3-methyl-5- (phenylethynyl)pyridin-2-yl)-1- ((tetrahydrofuran-3-yl)methyl)-1H- pyrazole-5-carboxamide | 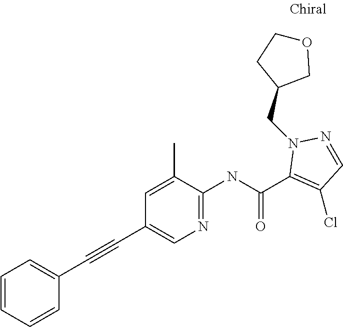 [SMILES: Cc1cc(C#Cc2ccccc2)cnc1NC(=O)c1c(Cl)cnn1C[C@H]1CCOC1] | 421 | 0.24 |
| 148 | (S)-4-chloro-N-(3-fluoro-5- (phenylethynyl)pyridin-2-yl)-1- ((tetrahydrofuran-3-yl)methyl)-1H- pyrazole-5-carboxamide | 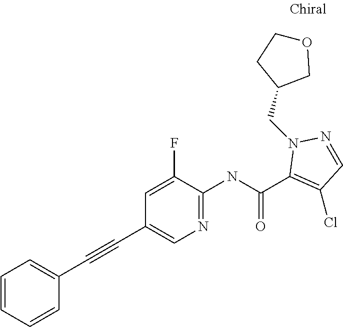 [SMILES: O=C(Nc1ncc(C#Cc2ccccc2)cc1F)c1c(Cl)cnn1C[C@@H]1CCOC1] | 425 | 0.24 |
| 149 | 4-chloro-1-(1- (cyclopropanecarbonyl)piperidin-4- yl)-N-(3-methyl-5- (phenylethynyl)pyridin-2-yl)-1H- pyrazole-5-carboxamide | 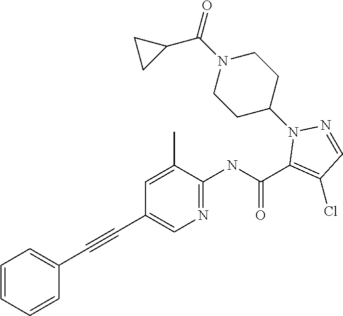 [SMILES: Cc1cc(C#Cc2ccccc2)cnc1NC(=O)c1c(Cl)cnn1C1CCN(C(=O)C2CC2)CC1] | 488.4 | 0.24 |
| 150 | 1-((1-acetylpiperidin-4-yl)methyl)-4- chloro-N-(5-((3- fluorophenyl)ethynyl)-3- methylpyridin-2-yl)-1H-pyrazole-5- carboxamide | 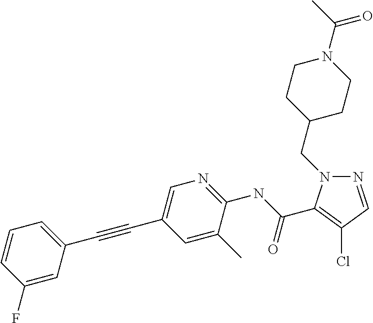 [SMILES: CC(=O)N1CCC(Cn2ncc(Cl)c2C(=O)Nc2ncc(C#Cc3cccc(F)c3)cc2C)CC1] | 494.4 | 0.24 |
| 151 | 4-chloro-N-(5-((4- fluorophenyl)ethynyl)-3- methylpyridin-2-yl)-1-(1- isobutyrylpiperidin-4-yl)-1H-pyrazole- 5-carboxamide | 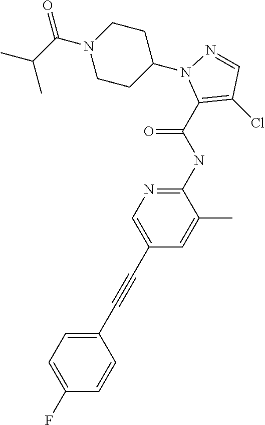 [SMILES: Cc1cc(C#Cc2ccc(F)cc2)cnc1NC(=O)c1c(Cl)cnn1C1CCN(C(=O)C(C)C)CC1] | 508 | 0.24 |
| 152 | 4-chloro-N-(5-((3- fluorophenyl)ethynyl)-3- methylpyridin-2-yl)-1-(1- isobutyrylpiperidin-4-yl)-1H-pyrazole- 5-carboxamide | 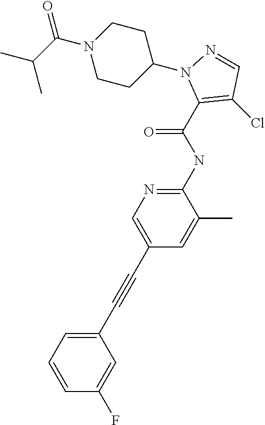 [SMILES: Cc1cc(C#Cc2cccc(F)c2)cnc1NC(=O)c1c(Cl)cnn1C1CCN(C(=O)C(C)C)CC1] | 508 | 0.24 |
| 153 | 1-(2-(1-acetylpiperidin-4-yl)ethyl)-4- chloro-N-(3-methyl-5- (phenylethynyl)pyridin-2-yl)-1H- pyrazole-5-carboxamide | 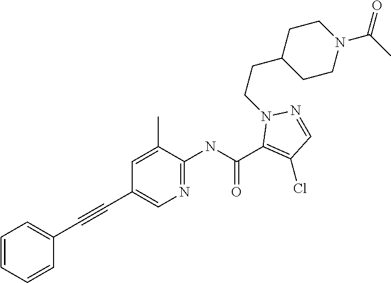 [SMILES: CC(=O)N1CCC(CCn2ncc(Cl)c2C(=O)Nc2ncc(C#Cc3ccccc3)cc2C)CC1] | 490.3 | 0.24 |
| 154 | 1-((3S,4S)-1-acetyl-3-fluoropiperidin- 4-yl)-4-chloro-N-(3-fluoro-5- (phenylethynyl)pyridin-2-yl)-1H- pyrazole-5-carboxamide | 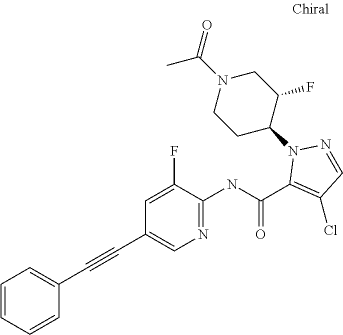 [SMILES: CC(=O)N1CC[C@H](n2ncc(Cl)c2C(=O)Nc2ncc(C#Cc3ccccc3)cc2F)[C@@H](F)C1] | 484.3 | 0.24 |
| 155 | tert-butyl 4-((4-chloro-5-((3-methyl-5- (phenylethynyl)pyridin-2- yl)carbamoyl)-1H-pyrazol-1- yl)methyl)-4-fluoropiperidine-1- carboxylate | 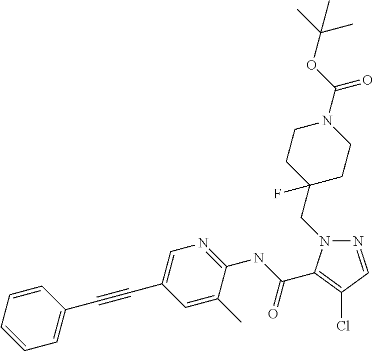 [SMILES: Cc1cc(C#Cc2ccccc2)cnc1NC(=O)c1c(Cl)cnn1CC1(F)CCN(C(=O)OC(C)(C)C)CC1] | 552.3 | 0.24 |
| 156 | 1-(trans-3-acetamidocyclobutyl)-4- chloro-N-(3-methyl-5- (phenylethynyl)pyridin-2-yl)-1H- pyrazole-5-carboxamide | 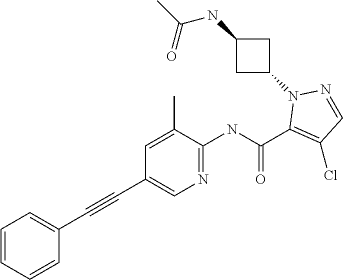 [SMILES: CC(=O)N[C@H]1C[C@H](n2ncc(Cl)c2C(=O)Nc2ncc(C#Cc3ccccc3)cc2C)C1] | 448.3 | 0.24 |
| 157 | (S)-4-chloro-N-(2-fluoro-6-methyl-4- (phenylethynyl)phenyl)-1- (tetrahydrofuran-3-yl)-1H-pyrazole-5- carboxamide | 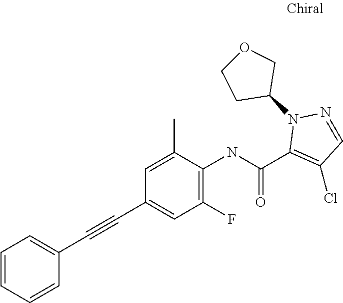 [SMILES: Cc1cc(C#Cc2ccccc2)cc(F)c1NC(=O)c1c(Cl)cnn1[C@H]1CCOC1] | 424 | 0.25 |
| 158 | 4-(4-chloro-5-((3-fluoro-5- (phenylethynyl)pyridin-2- yl)carbamoyl)-1H-pyrazol-1-yl)-N- ethylpiperidine-1-carboxamide | 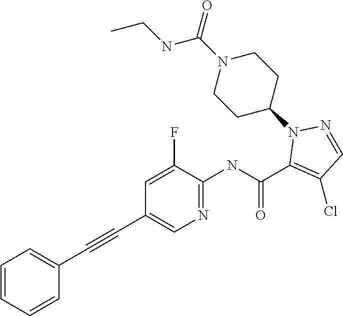 | 495.3 | 0.25 |
| 159 | 1-((3R,4R)-1-acetyl-3-fluoropiperidin- 4-yl)-4-chloro-N-(5-((2- fluorophenyl)ethynyl)-3- methylpyridin-2-yl)-1H-pyrazole-5- carboxamide | 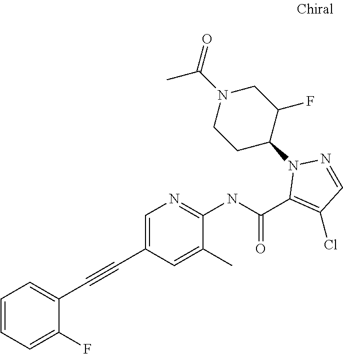 [SMILES: CC(=O)N1CC[C@H](n2ncc(Cl)c2C(=O)Nc2ncc(C#Cc3ccccc3F)cc2C)C(F)C1] | 498.3 | 0.25 |
| 160 | (S)-4-chloro-N-(3-methyl-5- (phenylethynyl)pyridin-2-yl)-1- ((tetrahydrofuran-3-yl)methyl)-1H- pyrazole-5-carboxamide | 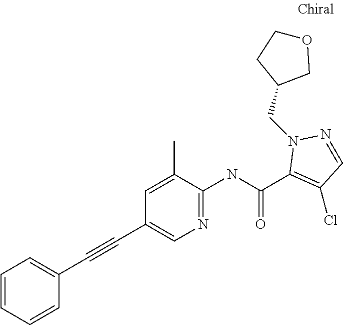 [SMILES: Cc1cc(C#Cc2ccccc2)cnc1NC(=O)c1c(Cl)cnn1C[C@@H]1CCOC1] | 421 | 0.26 |
| 161 | (S)-4-chloro-N-(2,6-difluoro-4- (phenylethynyl)phenyl)-1- (tetrahydrofuran-3-yl)methyl)-1H- pyrazole-5-carboxamide | 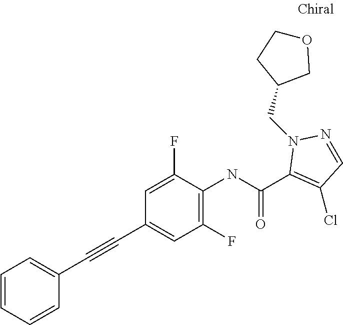 [SMILES: O=C(Nc1c(F)cc(C#Cc2ccccc2)cc1F)c1c(Cl)cnn1C[C@@H]1CCOC1] | 442 | 0.26 |
| 162 | (R)-4-chloro-N-(2,6-difluoro-4- (phenylethynyl)phenyl)-1- ((tetrahydrofuran-3-yl)methyl)-1H- pyrazole-5-carboxamide | 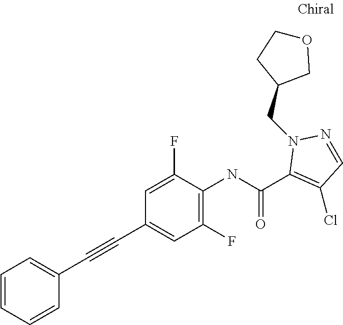 [SMILES: O=C(Nc1c(F)cc(C#Cc2ccccc2)cc1F)c1c(Cl)cnn1C[C@H]1CCOC1] | 442 | 0.26 |
| 163 | 4-chloro-N-(3-methyl-5- (phenylethynyl)pyridin-2-yl)-1-(1- (methylsulfonyl)piperidin-4-yl)-1H- pyrazole-5-carboxamide | 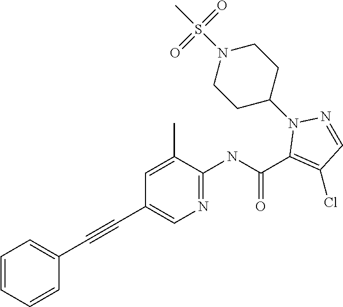 [SMILES: Cc1cc(C#Cc2ccccc2)cnc1NC(=O)c1c(Cl)cnn1C1CCN(S(C)(=O)=O)CC1] | 498 | 0.26 |
| 164 | 1-(1-acetylpiperidin-4-yl)-4-chloro-N- (5-((2-fluorophenyl)ethynyl)-3- methylpyridin-2-yl)-1H-pyrazole-5- carboxamide | 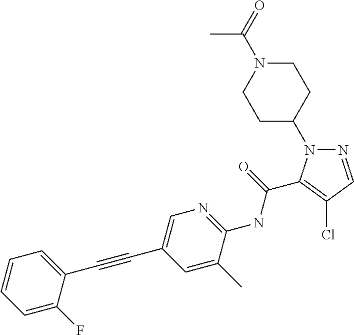 [SMILES: CC(=O)N1CCC(n2ncc(Cl)c2C(=O)Nc2ncc(C#Cc3ccccc3F)cc2C)CC1] | 480.4 | 0.26 |
| 165 | 4-chloro-1-(cis-4- (cyclopropanecarboxamido)cyclohexyl)- N-(5-((4-fluorophenyl)ethynyl)-3- methylpyridin-2-yl)-1H-pyrazole-5- carboxamide | 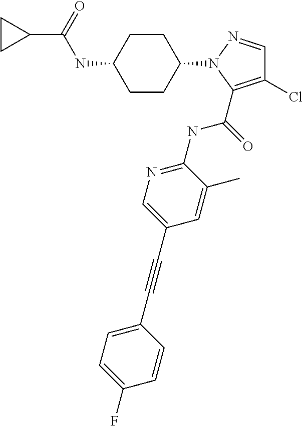 [SMILES: Cc1cc(C#Cc2ccc(F)cc2)cnc1NC(=O)c1c(Cl)cnn1[C@H]1CC[C@@H](NC(=O)C2CC2)CC1] | 520.4 | 0.26 |
| 166 | 1-((1-acetyl-4-methylpiperidin-4- yl)methyl)-4-chloro-N-(3-methyl-5- (phenylethynyl)pyridin-2-yl)-1H- pyrazole-5-carboxamide | 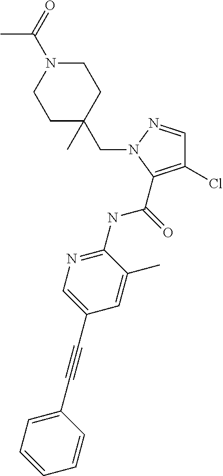 [SMILES: CC(=O)N1CCC(C)(Cn2ncc(Cl)c2C(=O)Nc2ncc(C#Cc3ccccc3)cc2C)CC1] | 490.4 | 0.26 |
| 167 | 4-chloro-1-(3-cyanopropyl)-N-(3- methyl-5-(phenylethynyl)pyridin-2- yl)-1H-pyrazole-5-carboxamide | 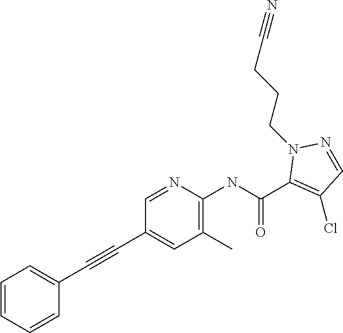 [SMILES: Cc1cc(C#Cc2ccccc2)cnc1NC(=O)c1c(Cl)cnn1CCCC#N] | 404 | 0.27 |
| 168 | 4-chloro-1-((1- cyanocyclopropyl)methyl)-N-(2,6- difluoro-4-(phenylethynyl)phenyl)- 1H-pyrazole-5-carboxamide | 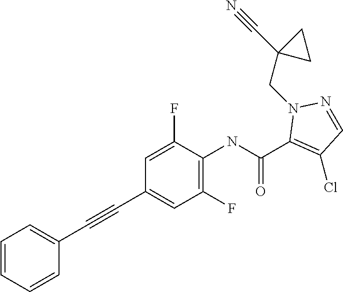 [SMILES: N#CC1(Cn2ncc(Cl)c2C(=O)Nc2c(F)cc(C#Cc3ccccc3)cc2F)CC1] | 437 | 0.27 |
| 169 | 4-chloro-1-(1-isobutyrylpiperidin-4- yl)-N-(3-methyl-5- phenylethynyl)pyridin-2-yl)-1H- pyrazole-5-carboxamide | 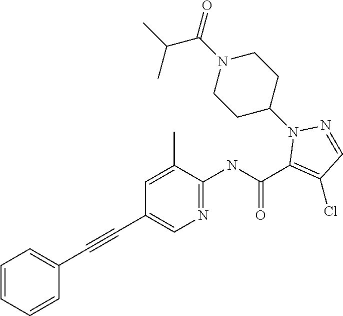 [SMILES: Cc1cc(C#Cc2ccccc2)cnc1NC(=O)c1c(Cl)cnn1C1CCN(C(=O)C(C)C)CC1] | 490.3 | 0.27 |
| 170 | tert-butyl (3aR,5s,6aS)-5-(4-chloro-5- {[3-fluoro-5-(phenylethynyl)pyridin- 2-yl]carbamoyl}-1H-pyrazol-1- yl)hexahydrocyclopenta[c]pyrrole- 2(1H)-carboxylate | 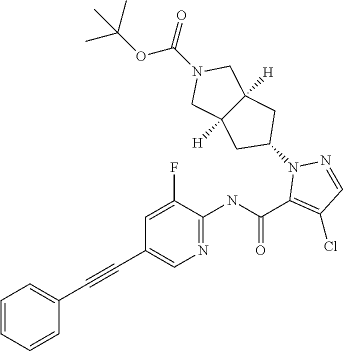 [SMILES: CC(C)(C)OC(=O)N1C[C@H]2C[C@H](n3ncc(Cl)c3C(=O)Nc3ncc(C#Cc4ccccc4)cc3F)C[C@H]2C1] | 550.1 | 0.27 |
| 171 | 4-chloro-N-(3-methyl-5- (phenylethynyl)pyridin-2-yl)-1-(1- (3,3,3-trifluoropropanoyl)piperidin-4- yl)-1H-pyrazole-5-carboxamide | 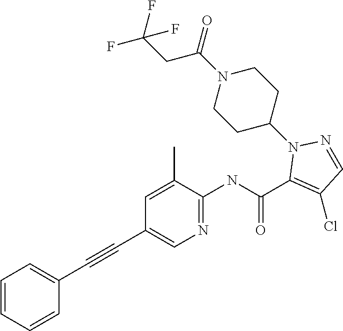 [SMILES: Cc1cc(C#Cc2ccccc2)cnc1NC(=O)c1c(Cl)cnn1C1CCN(C(=O)CC(F)(F)F)CC1] | 530.2 | 0.27 |
| 172 | 4-chloro-1-(2-(1- (cyclopropanecarbonyl)piperidin-4- yl)ethyl)-N-(3-methyl-5- (phenylethynyl)pyridin-2-yl)-1H- pyrazole-5-carboxamide | 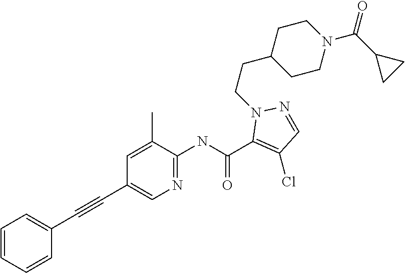 [SMILES: Cc1cc(C#Cc2ccccc2)cnc1NC(=O)c1c(Cl)cnn1CCC1CCN(C(=O)C2CC2)CC1] | 516.4 | 0.27 |
| 173 | 1-(2-acetyl-2-azaspiro[3.3]heptan-6- yl)-4-chloro-N-(3-methyl-5- (phenylethynyl)pyridin-2-yl)-1H- pyrazole-5-carboxamide | 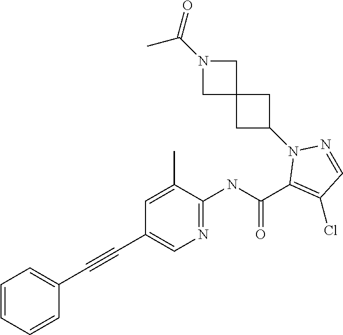 [SMILES: CC(=O)N1CC2(CC(n3ncc(Cl)c3C(=O)Nc3ncc(C#Cc4ccccc4)cc3C)C2)C1] | 474.3 | 0.27 |
| 174 | 4-chloro-1-((1- (cyclopropanecarbonyl)piperidin-4- yl)methyl)-N-(5-((3- fluorophenyl)ethynyl)-3- methylpyridin-2-yl)-1H-pyrazole- 5-carboxamide | 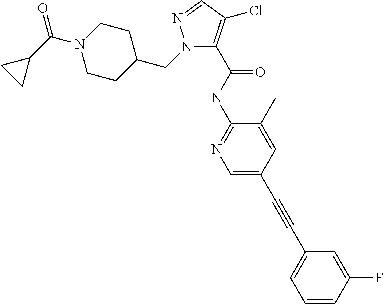 [SMILES: Cc1cc(C#Cc2cccc(F)c2)cnc1NC(=O)c1c(Cl)cnn1CC1CCN(C(=O)C2CC2)CC1] | 520 | 0.27 |
| 175 | 4-chloro-N-(3-fluoro-5-(thiophen-2- ylethynyl)pyridin-2-yl)-1-(1- isobutyrylpiperidin-4-yl)-1H- pyrazole-5-carboxamide | 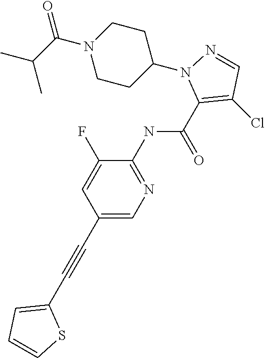 [SMILES: CC(C)C(=O)N1CCC(n2ncc(Cl)c2C(=O)Nc2ncc(C#Cc3cccs3)cc2F)CC1] | 500.3 | 0.27 |
| 176 | 1-((1-acetylpiperdin-3-yl)methyl)-4- chloro-N-(3-methyl-5- (phenylethynyl)pyridin-2-yl)-1H- pyrazole-5-carboxamide | 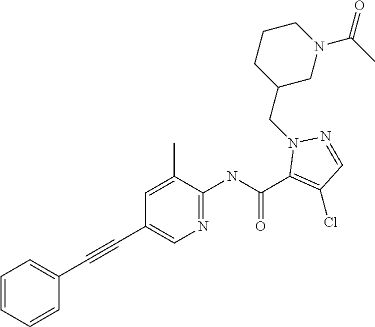 [SMILES: CC(=O)N1CCCC(Cn2ncc(Cl)c2C(=O)Nc2ncc(C#Cc3ccccc3)cc2C)C1] | 476.3 | 0.27 |
| 177 | 1-((1-acetylpiperidin-3-yl)methyl)-4- chloro-N-(3-fluoro-5- (phenylethynyl)pyridin-2-yl)-1H- pyrazole-5-carboxamide | 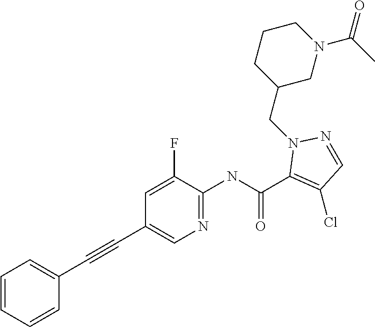 [SMILES: CC(=O)N1CCCC(Cn2ncc(Cl)c2C(=O)Nc2ncc(C#Cc3ccccc3)cc2F)C1] | 480.2 | 0.27 |
| 178 | 1-(((3S,4S)-1-acetyl-3-fluoropiperidin- 4-yl)methyl)-4-chloro-N-(3-fluoro-5- (phenylethynyl)pyridin-2-yl)-1H- pyrazole-5-carboxamide | 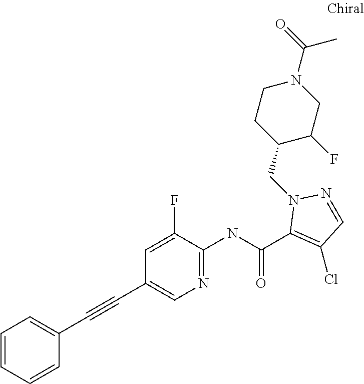 [SMILES: CC(=O)N1CC[C@@H](Cn2ncc(Cl)c2C(=O)Nc2ncc(C#Cc3ccccc3)cc2F)C(F)C1] | 498.3 | 0.27 |
| 179 | 1-((1-acetyl-4-cyanopiperidin-4- yl)methyl)-4-chloro-N-(5-((4- fluorophenyl)ethynyl)-3- methylpyridin-2-yl)-1H-pyrazole-5- carboxamide | 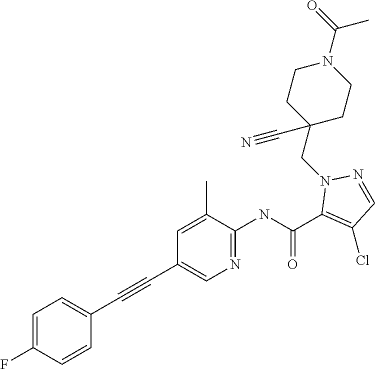 [SMILES: CC(=O)N1CCC(C#N)(Cn2ncc(Cl)c2C(=O)Nc2ncc(C#Cc3ccc(F)cc3)cc2C)CC1] | 519.2 | 0.27 |
| 180 | 4-chloro-N-(2-fluoro-6-methyl-4- (phenylethynyl)phenyl)-1-(tetrahydro- 2H-pyran-4-yl)-1H-pyrazole-5- carboxamide | 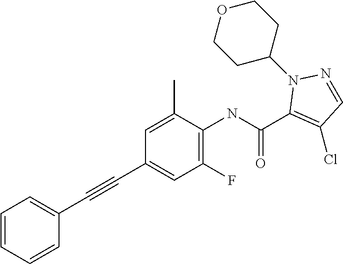 [SMILES: Cc1cc(C#Cc2ccccc2)cc(F)c1NC(=O)c1c(Cl)cnn1C1CCOCC1] | 438 | 0.28 |
| 181 | 4-chloro-N-(5-((4- fluorophenyl)ethynyl)-3- methylpyridin-2-yl)-1-(1- pivaloylpiperidin-4-yl)-1H-pyrazole-5- carboxamide | 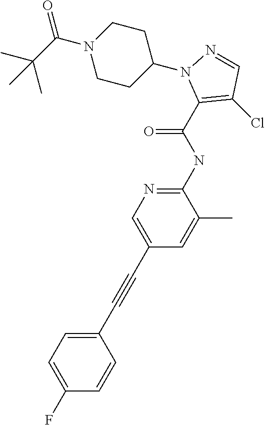 [SMILES: Cc1cc(C#Cc2ccc(F)cc2)cnc1NC(=O)c1c(Cl)cnn1C1CCN(C(=O)C(C)(C)C)CC1] | 522 | 0.28 |
| 182 | 4-chloro-N-(3-fluoro-5- (phenylethynyl)pyridin-2-yl)-1-(2- isobutyryl-2-azaspiro[3.3]heptan-6- yl)-1H-pyrazole-5-carboxamide | 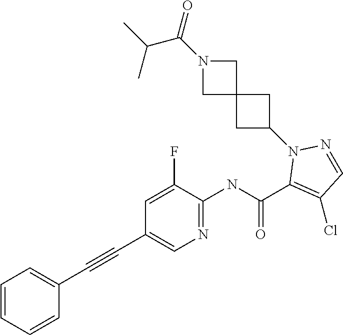 [SMILES: CC(C)C(=O)N1CC2(CC(n3ncc(Cl)c3C(=O)Nc3ncc(C#Cc4ccccc4)cc3F)C2)C1] | 506.4 | 0.28 |
| 183 | 4-chloro-1-(trans-3- isobutyramidocyclobutyl)-N-(3- methyl-5-(phenylethynyl)pyridin-2- yl)-1H-pyrazole-5-carboxamide | 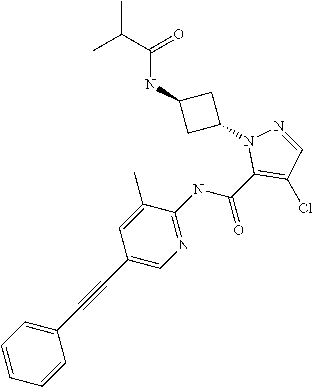 [SMILES: Cc1cc(C#Cc2ccccc2)cnc1NC(=O)c1c(Cl)cnn1[C@H]1C[C@H](NC(=O)C(C)C)C1] | 476.4 | 0.28 |
| 184 | 4-chloro-1-((4-methyl-1-(2- methylcyclopropane-1- carbonyl)piperidin-4-yl)methyl)-N- (3-methyl-5-(phenylethynyl)pyridin-2- yl)-1H-pyrazole-5-carboxamide | 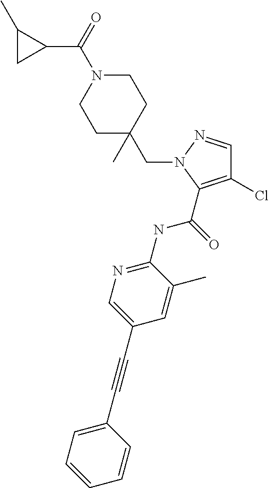 [SMILES: Cc1cc(C#Cc2ccccc2)cnc1NC(=O)c1c(Cl)cnn1CC1(C)CCN(C(=O)C2CC2C)CC1] | 530.4 | 0.28 |
| 185 | 4-chloro-1-(3-cyanopropyl)-N-(2- fluoro-6-methyl-4- (phenylethynyl)phenyl)-1H-pyrazole- 5-carboxamide | 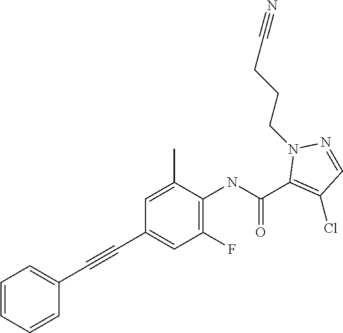 [SMILES: Cc1cc(C#Cc2ccccc2)cc(F)c1NC(=O)c1c(Cl)cnn1CCCC#N] | 421 | 0.29 |
| 186 | tert-butyl 4-((4-chloro-5-((3-methyl-5- (phenylethynyl)pyridin-2- yl)carbamoyl)-1H-pyrazol-1- yl)methyl)piperidine-1-carboxylate | 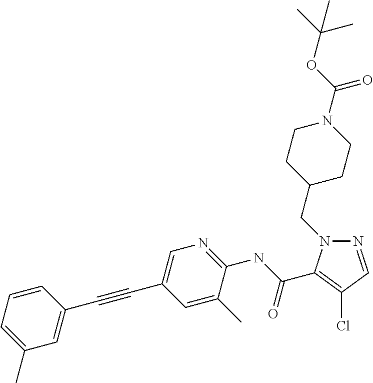 [SMILES: Cc1cccc(C#Cc2cnc(NC(=O)c3c(Cl)cnn3CC3CCN(C(=O)OC(C)(C)C)CC3)c(C)c2)c1] | 534.3 | 0.29 |
| 187 | 4-chloro-1-[(3aR,5s,6aS)-2-(2- methylpropanoyl)octahydrocyclopenta [c]pyrrol-5-yl]-N-{3-methyl-5- [(thiophen-2-yl)ethynyl]pyridin-2-yl}- 1H-pyrazole-5-carboxamide | 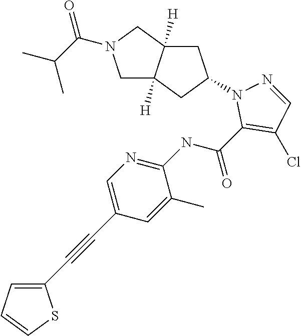 [SMILES: Cc1cc(C#Cc2cccs2)cnc1NC(=O)c1c(Cl)cnn1[C@H]1C[C@@H]2CN(C(=O)C(C)C)C[C@@H]2C1] | 522.4 | 0.29 |
| 188 | 4-chloro-1-(2-(1-isobutyrylpiperidin- 4-yl)ethyl)-N-(3-methyl-5- (phenylethynyl)pyridin-2-yl)-1H- pyrazole-5-carboxamide | 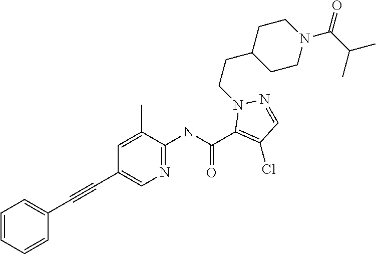 [SMILES: Cc1cc(C#Cc2ccccc2)cnc1NC(=O)c1c(Cl)cnn1CCC1CCN(C(=O)C(C)C)CC1] | 518.4 | 0.29 |
| 189 | 4-chloro-1-(2,3- dihydrobenzo[b][1,4]dioxin-6-yl)-N- (3-fluoro-5-(phenylethynyl)pyridin-2- yl)-1H-pyrazole-5-carboxamide | 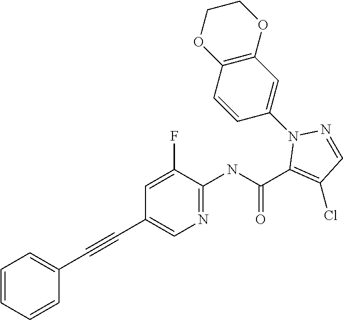 [SMILES: O=C(Nc1ncc(C#Cc2ccccc2)cc1F)c1c(Cl)cnn1-c1ccc2c(c1)OCCO2] | 475 | 0.29 |
| 190 | 4-chloro-1-((4-fluoro-1- isobutyrylpiperidin-4-yl)methyl)-N-(3- fluoro-5-(phenylethynyl)pyridin-2-yl)- 1H-pyrazole-5-carboxamide | 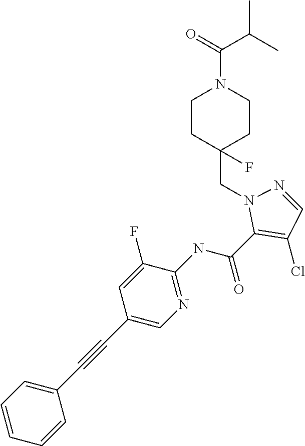 [SMILES: CC(C)C(=O)N1CCC(F)(Cn2ncc(Cl)c2C(=O)Nc2ncc(C#Cc3ccccc3)cc2F)CC1] | 526.2 | 0.29 |
| 191 | 4-chloro-1-(trans-4- isobutyramidocyclohexyl)-N-(3- methyl-5-(phenylethynyl)pyridin-2- yl)-1H-pyrazole-5-carboxamide | 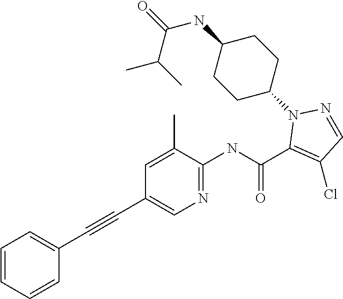 [SMILES: Cc1cc(C#Cc2ccccc2)cnc1NC(=O)c1c(Cl)cnn1[C@H]1CC[C@H](NC(=O)C(C)C)CC1] | 504.3 | 0.29 |
| 192 | 1-((1-acetyl-3,3-difluoropiperidin-4- yl)methyl)-4-chloro-N-(3-fluoro-5- (phenylethynyl)pyridin-2-yl)-1H- pyrazole-5-carboxamide | 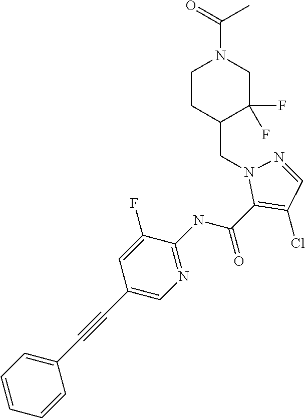 [SMILES: CC(=O)N1CCC(Cn2ncc(Cl)c2C(=O)Nc2ncc(C#Cc3ccccc3)cc2F)C(F)(F)C1] | 516.2 | 0.29 |
| 193 | 1-(trans-4-acetamidocyclohexyl)-4- chloro-N-(5-((4- fluorophenyl)ethynyl)-3- methylpyridin-2-yl)-1H-pyrazole-5- carboxamide | 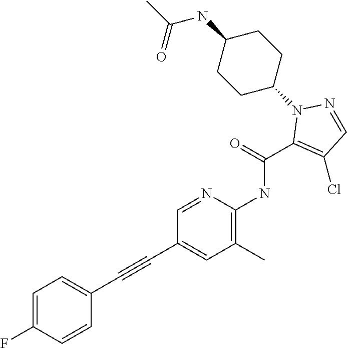 [SMILES: CC(=O)N[C@H]1CC[C@H](n2ncc(Cl)c2C(=O)Nc2ncc(C#Cc3ccc(F)cc3)cc2C)CC1] | 494.2 | 0.29 |
| 194 | 4-chloro-1-(3-cyanocyclobutyl)-N-(2- fluoro-6-methyl-4- (phenylethynyl)phenyl)-1H-pyrazole- 5-carboxamide | 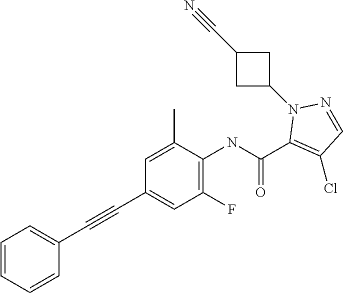 [SMILES: Cc1cc(C#Cc2ccccc2)cc(F)c1NC(=O)c1c(Cl)cnn1C1CC(C#N)C1] | 433 | 0.3 |
| 195 | 1-[(3aR,5s,6aS)-2- acetyloctahydrocyclopenta[c]pyrrol-5- yl]-4-chloro-N-{5-[(3- fluorophenyl)ethynyl]-3- methylpyridin-2-yl}-1H-pyrazole-5- carboxamide | 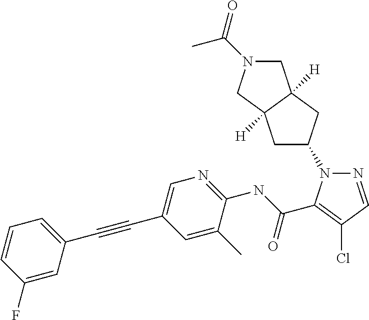 [SMILES: CC(=O)N1C[C@H]2C[C@H](n3ncc(Cl)c3C(=O)Nc3ncc(C#Cc4cccc(F)c4)cc3C)C[C@H]2C1] | 506.4 | 0.3 |
| 196 | 1-(2-(1-acetylpiperidin-4-yl)ethyl)-4- chloro-N-(5-((2- fluorophenyl)ethynyl)-3- methylpyridin-2-yl)-1H-pyrazole-5- carboxamide | 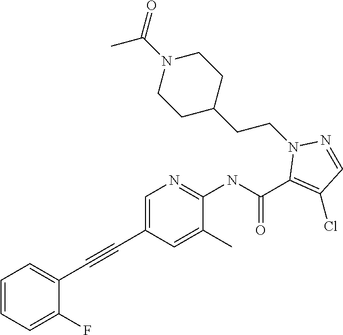 [SMILES: CC(=O)N1CCC(CCn2ncc(Cl)c2C(=O)Nc2ncc(C#Cc3ccccc3F)cc2C)CC1] | 508 | 0.3 |
| 197*³ | 1-[(1R)-1-(1-acetylpiperidin-4- yl)ethyl]-4-chloro-N-[3-methyl-5- (phenylethynyl)pyridin-2-yl]-1H- pyrazole-5-carboxamide or 1-[(1S)-1-(1-acetylpiperidin-4- yl)ethyl]-4-chloro-N-[3-methyl-5- (phenylethynyl)pyridin-2-yl]-1H- pyrazole-5-carboxamide | 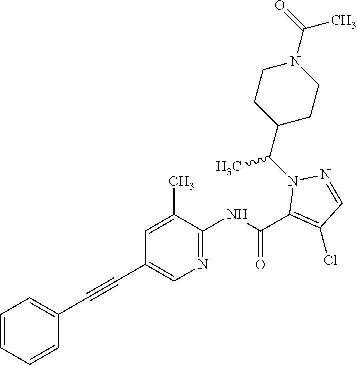 [SMILES: CC(=O)N1CCC(C(C)n2ncc(Cl)c2C(=O)Nc2ncc(C#Cc3ccccc3)cc2C)CC1] | 490.3 | 0.3 |
| 198 | 4-chloro-1-(trans-3- (cyclopropanecarboxamido)cyclobutyl)- N-(5-((4-fluorophenyl)ethynyl)-3- methylpyridin-2-yl)-1H-pyrazole-5- carboxamide | 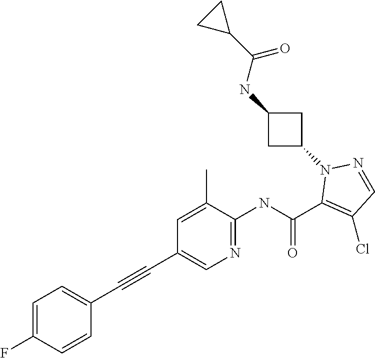 [SMILES: Cc1cc(C#Cc2ccc(F)cc2)cnc1NC(=O)c1c(Cl)cnn1[C@H]1C[C@H](NC(=O)C2CC2)C1] | 492.3 | 0.3 |
| 199 | (S)-4-chloro-N-(2,6-difluoro-4- (phenylethnyl)phenyl)-1- (tetrahydrofuran-3-yl)-1H-pyrazole-5- carboxamide | 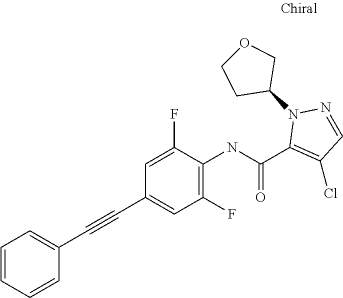 [SMILES: O=C(Nc1c(F)cc(C#Cc2ccccc2)cc1F)c1c(Cl)cnn1[C@H]1CCOC1] | 428 | 0.31 |
| 200 | (S)-4-chloro-N-(2-fluoro-6-methyl-4- (phenylethynyl)phenyl)-1- ((tetrahydrofuran-3-yl)methyl)-1H- pyrazole-5-carboxamide | 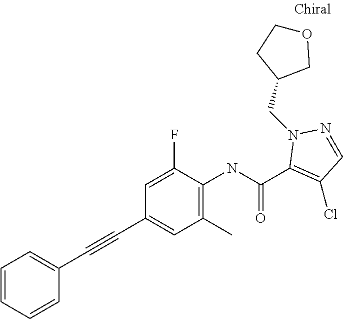 [SMILES: Cc1cc(C#Cc2ccccc2)cc(F)c1NC(=O)c1c(Cl)cnn1C[C@@H]1CCOC1] | 438 | 0.31 |
| 201 | 4-chloro-N-(2,6-difluoro-4- (phenylethynyl)phenyl)-1-(tetrahydro- 2H-pyran-4-yl)-1H- pyrazole-5-carboxamide | 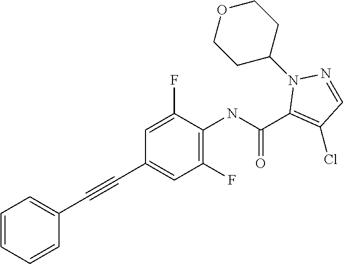 [SMILES: O=C(Nc1c(F)cc(C#Cc2ccccc2)cc1F)c1c(Cl)cnn1C1CCOCC1] | 442 | 0.31 |
| 202 | (R)-1-(1-acetylpyrrolidin-3-yl)-4- chloro-N-(3-methyl-5- (phenylethynyl)pyridin-2-yl)-1H- pyrazole-5-carboxamide | 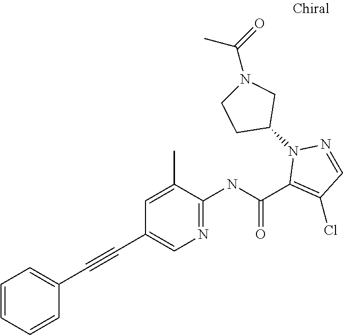 [SMILES: CC(=O)N1CC[C@@H](n2ncc(Cl)c2C(=O)Nc2ncc(C#Cc3ccccc3)cc2C)C1] | 448 | 0.31 |
| 203 | (R)-1-(1-acetylpyrrolidin-3-yl)-4- chloro-N-(3-fluoro-5- (phenylethynyl)pyridin-2-yl)-1H- pyrazole-5-carboxamide | 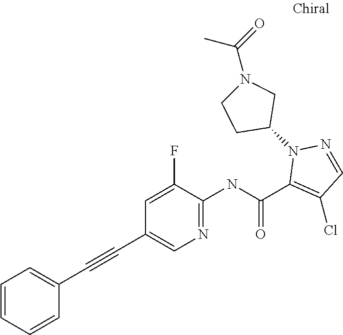 [SMILES: CC(=O)N1CC[C@@H](n2ncc(Cl)c2C(=O)Nc2ncc(C#Cc3ccccc3)cc2F)C1] | 452 | 0.31 |
| 204 | 4-chloro-1-[(3aR,5s,6aS)-2- (cyclopropanecarbonyl)octahydrocyclo- penta[c]pyrrol-5-yl]-N-{3-methyl-5- [(thiophen-2-yl)ethynyl]pyridin-2-yl}- 1H-pyrazole-5-carboxamide | 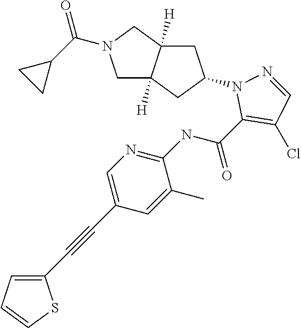 [SMILES: Cc1cc(C#Cc2cccs2)cnc1NC(=O)c1c(Cl)cnn1[C@H]1C[C@@H]2CN(C(=O)C3CC3)C[C@@H]2C1] | 520.2 | 0.31 |
| 205 | 4-chloro-N-(5-((3- fluorophenyl)ethynyl)-3- methylpyridin-2-yl)-1-(1- pivaloylpiperidin-4-yl)- 1H-pyrazole-5- carboxamide | 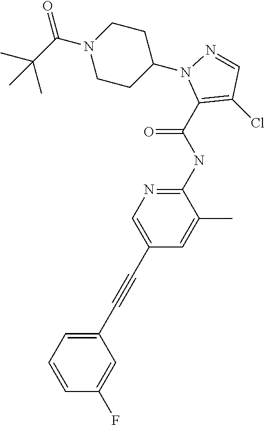 [SMILES: Cc1cc(C#Cc2cccc(F)c2)cnc1NC(=O)c1c(Cl)cnn1C1CCN(C(=O)C(C)(C)C)CC1] | 522 | 0.31 |
| 206 | 1-(2-(1-(1,4-dioxane-2- carbonyl)piperidin-4-yl)ethyl)-4- chloro-N-(3-fluoro-5-(thiophen-2- ylethynyl)pyridin-2-yl)-1H-pyrazole- 5-carboxamide | 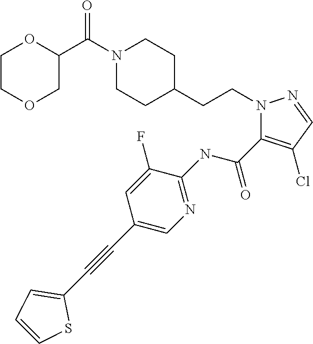 [SMILES: O=C(Nc1ncc(C#Cc2cccs2)cc1F)c1c(Cl)cnn1CCC1CCN(C(=O)C2COCCO2)CC1] | 572.2 | 0.31 |
| 207 | 4-chloro-1-(cis-3- isobutyramidocyclobutyl)-N-(3- methyl-5-(phenylethynyl)pyridin-2- yl)-1H-pyrazole-5-carboxamide | 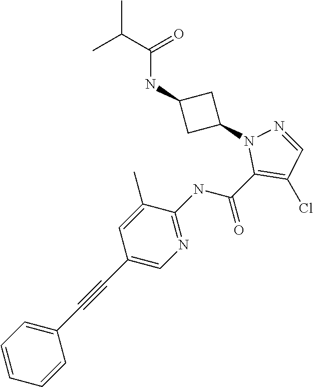 [SMILES: Cc1cc(C#Cc2ccccc2)cnc1NC(=O)c1c(Cl)cnn1[C@H]1C[C@@H](NC(=O)C(C)C)C1] | 476.2 | 0.31 |
| 208 | 1-(((3S,4S)-1-acetyl-3-fluoropiperidin- 4-yl)methyl)-4-chloro-N-(3-methyl-5- (phenylethynyl)pyridin-2-yl)-1H- pyrazole-5-carboxamide | 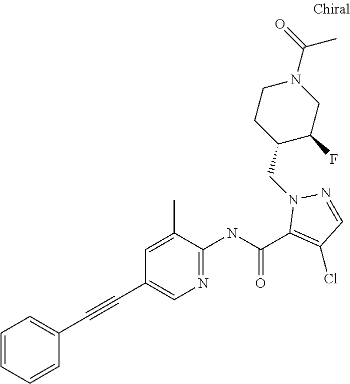 [SMILES: CC(=O)N1CC[C@@H](Cn2ncc(Cl)c2C(=O)Nc2ncc(C#Cc3ccccc3)cc2C)[C@H](F)C1] | 494.3 | 0.31 |
| 209 | 4-chloro-N-(5-((4- fluorophenyl)ethynyl)-3- methylpyridin-2-yl)-1-(trans-3- isobutyramidocyclobutyl)-1H- pyrazole-5-carboxamide | 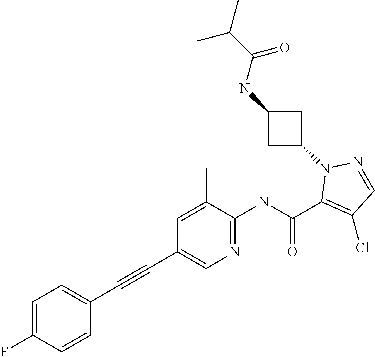 [SMILES: Cc1cc(C#Cc2ccc(F)cc2)cnc1NC(=O)c1c(Cl)cnn1[C@H]1C[C@H](NC(=O)C(C)C)C1] | 494.3 | 0.31 |
| 210 | 4-chloro-N-(3-methyl-5- (phenylethynyl)pyridin-2-yl)-1- (tetrahydro-2H-pyran-4-yl)-1H- pyrazole-5-carboxamide | 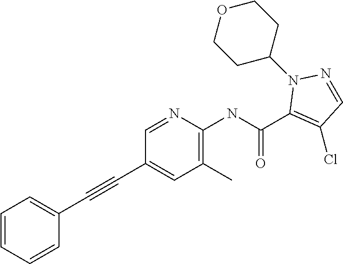 [SMILES: Cc1cc(C#Cc2ccccc2)cnc1NC(=O)c1c(Cl)cnn1C1CCOCC1] | 421 | 0.32 |
| 211 | (S)-1-((1,4-dioxan-2-yl)methyl)-4- chloro-N-(3-methyl-5- (phenylethynyl)pyridin-2-yl)-1H- pyrazole-5-carboxamide | 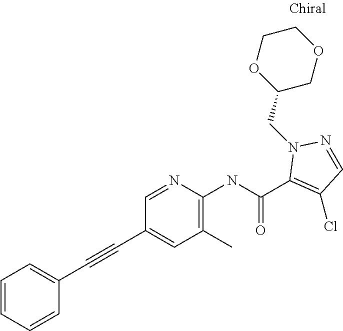 [SMILES: Cc1cc(C#Cc2ccccc2)cnc1NC(=O)c1c(Cl)cnn1C[C@H]1COCCO1] | 437 | 0.32 |
| 212 | 4-chloro-1-(2-isobutyryl-2- azaspiro[3.3]heptan-6-yl)-N-(3- methyl-5-(phenylethynyl)pyridin-2- yl)-1H-pyrazole-5-carboxamide | 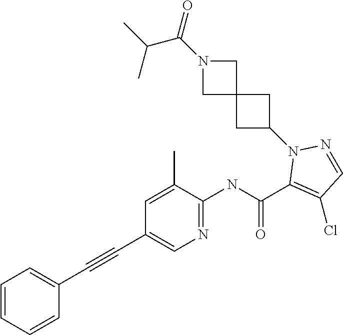 [SMILES: Cc1cc(C#Cc2ccccc2)cnc1NC(=O)c1c(Cl)cnn1C1CC2(C1)CN(C(=O)C(C)C)C2] | 502.3 | 0.33 |
| 213 | 1-((3R,4R)-1-acetyl-3-fluoropiperidin- 4-yl)-4-chloro-N-(3-fluoro-5-((4- fluorophenyl)ethynyl)pyridin-2-yl)- 1H-pyrazole-5-carboxamide | 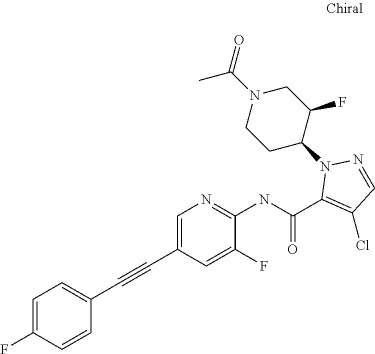 [SMILES: CC(=O)N1CC[C@H](n2ncc(Cl)c2C(=O)Nc2ncc(C#Cc3ccc(F)cc3)cc2F)[C@H](F)C1] | 502.3 | 0.33 |
| 214 | tert-butyl (trans-3-(4-chloro-5-((3- fluoro-5-(phenylethynyl)pyridin-2- yl)carbamoyl)-1H-pyrazol-1- yl)cyclobutyl)carbamate | 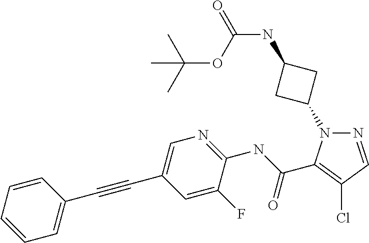 [SMILES: CC(C)(C)OC(=O)N[C@H]1C[C@H](n2ncc(Cl)c2C(=O)Nc2ncc(C#Cc3ccccc3)cc2F)C1] | 510.3 | 0.33 |
| 215 | 1-((1-acetyl-3-fluoroazetidin-3- yl)methyl)-4-chloro-N-(3-methyl-5- (phenylethynyl)pyridin-2-yl)-1H- pyrazole-5-carboxamide | 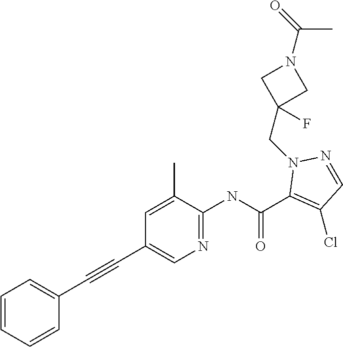 [SMILES: CC(=O)N1CC(F)(Cn2ncc(Cl)c2C(=O)Nc2ncc(C#Cc3ccccc3)cc2C)C1] | 466.3 | 0.33 |
| 216 | 4-chloro-1-((3,3-difluoro-1- isobutyrylpiperidin-4-yl)methyl)-N-(3- fluoro-5-(phenylethynyl)pyridin-2-yl)- 1H-pyrazole-5-carboxamide | 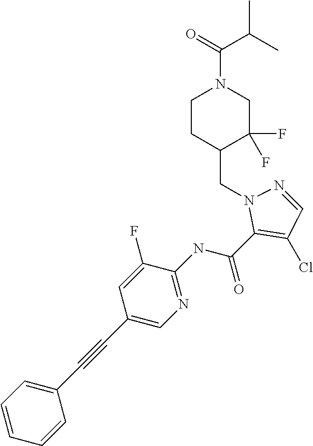 [SMILES: CC(C)C(=O)N1CCC(Cn2ncc(Cl)c2C(=O)Nc2ncc(C#Cc3ccccc3)cc2F)C(F)(F)C1] | 544.2 | 0.33 |
| 217 | 4-chloro-N-(3-fluoro-5- (phenylethynyl)pyridin-2-yl)-1- ((tetrahydrofuran-3-yl)methyl)-1H- pyrazole-5-carboxamide | 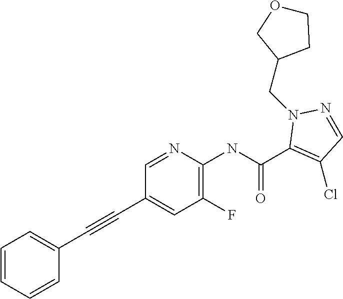 [SMILES: O=C(Nc1ncc(C#Cc2ccccc2)cc1F)c1c(Cl)cnn1CC1CCOC1] | 425.2 | 0.34 |
| 218 | (S)-4-chloro-1-(2-methoxypropyl)-N- (3-methyl-5-(phenylethynyl)pyridin-2- yl)-1H-pyrazole-5-carboxamide | 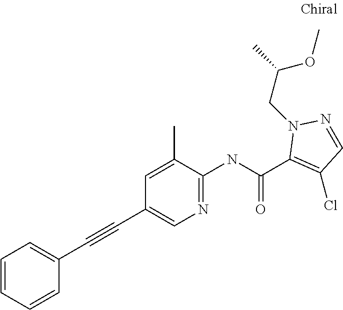 [SMILES: CO[C@@H](C)Cn1ncc(Cl)c1C(=O)Nc1ncc(C#Cc2ccccc2)cc1C] | 409 | 0.34 |
| 219 | (R)-4-chloro-N-(2-fluoro-6-methyl-4- (phenylethynyl)phenyl)-1- ((tetrahydrofuran-3-yl)methyl)-1H- pyrazole-5-carboxamide | 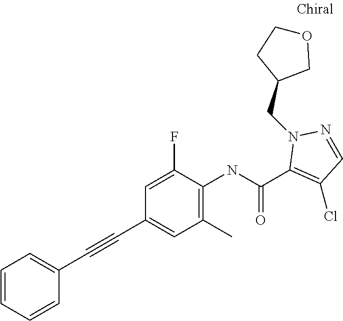 [SMILES: Cc1cc(C#Cc2ccccc2)cc(F)c1NC(=O)c1c(Cl)cnn1C[C@H]1CCOC1] | 438 | 0.34 |
| 220 | 4-chloro-N-(3-methyl-5- (phenylethynyl)pyridin-2-yl)-1-(1- pivaloylpiperidin-4-yl)-1H-pyrazole-5- carboxamide | 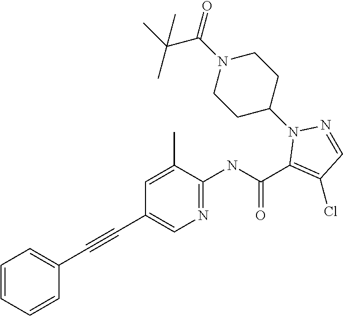 [SMILES: Cc1cc(C#Cc2ccccc2)cnc1NC(=O)c1c(Cl)cnn1C1CCN(C(=O)C(C)(C)C)CC1] | 504.4 | 0.34 |
| 221 | tert-butyl ((1R,3R)-3-(4-chloro-5-((3- fluoro-5-(phenylethynyl)pyridin-2- yl)carbamoyl)-1H-pyrazol-1- yl)cyclopentyl)carbamate | 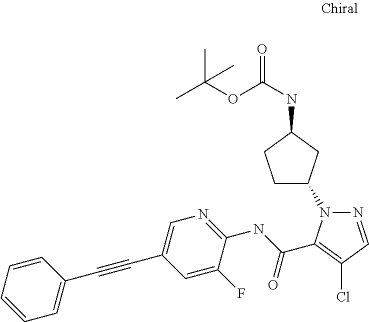 [SMILES: CC(C)(C)OC(=O)N[C@@H]1CC[C@@H](n2ncc(Cl)c2C(=O)Nc2ncc(C#Cc3ccccc3)cc2F)C1] | 524.3 | 0.34 |
| 222 | 4-chloro-1-(((3S,4S)-3-fluoro-1- isobutyrylpiperidin-4-yl)methyl)-N-(3- methyl-5-(phenylethynyl)pyridin-2- yl)-1H-pyrazole-5-carboxamide | 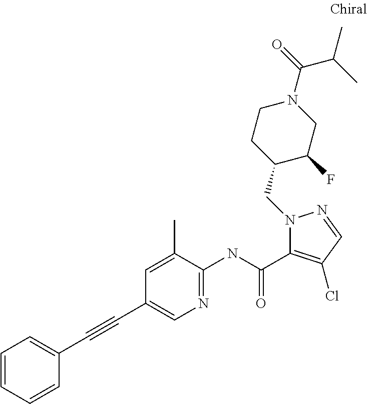 [SMILES: Cc1cc(C#Cc2ccccc2)cnc1NC(=O)c1c(Cl)cnn1C[C@@H]1CCN(C(=O)C(C)C)C[C@H]1F] | 522.2 | 0.34 |
| 223 | 1-((1-(1,4-dioxane-2- carbonyl)piperidin-3-yl)methyl)-4- chloro-N-(5-((4- fluorophenyl)ethynyl)-3- methylpyridin-2-yl)-1H-pyrazole-5- carboxamide | 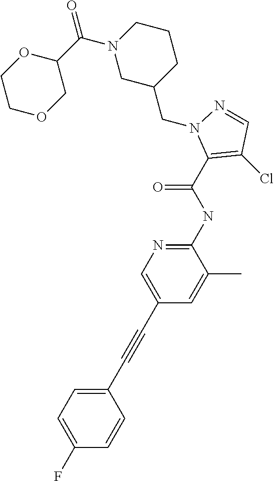 [SMILES: Cc1cc(C#Cc2ccc(F)cc2)cnc1NC(=O)c1c(Cl)cnn1CC1CCCN(C(=O)C2COCCO2)C1] | 566.4 | 0.34 |
| 224 | (R)-4-chloro-N-(3-methyl-5- (phenylethynyl)pyridin-2-yl)-1- (tetrahydrofuran-3-yl)-1H-pyrazole-5- carboxamide | 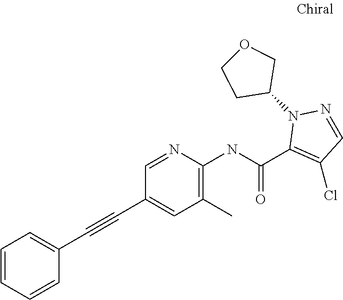 [SMILES: Cc1cc(C#Cc2ccccc2)cnc1NC(=O)c1c(Cl)cnn1[C@@H]1CCOC1] | 407 | 0.75 |
| 225 | tert-butyl 4-(4-chloro-5-((3-fluoro-5- (phenylethynyl)pyridin-2- yl)carbamoyl)-1H-pyrazol-1- yl)azepane-1-carboxylate | 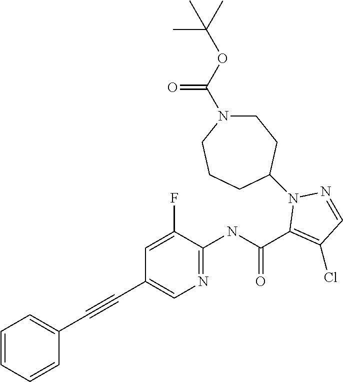 [SMILES: CC(C)(C)OC(=O)N1CCCC(n2ncc(Cl)c2C(=O)Nc2ncc(C#Cc3ccccc3)cc2F)CC1] | 482.3 (M − tBu + 2H) | 0.35 |
| 226 | 4-chloro-1-(2-(1- (cyclopropanecarbonyl)piperidin-4- yl)ethyl)-N-(5-((2- fluorophenyl)ethynyl)-3- methylpyridin-2-yl)-1H- pyrazole-5-carboxamide | 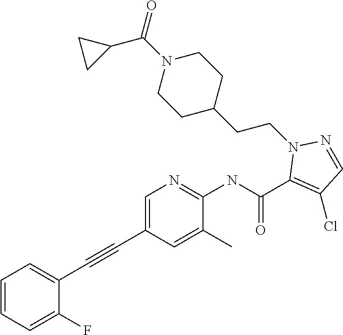 [SMILES: Cc1cc(C#Cc2ccccc2F)cnc1NC(=O)c1c(Cl)cnn1CCC1CCN(C(=O)C2CC2)CC1] | 534 | 0.35 |
| 227 | 1-(benzo[d][1,3]dioxol-5-yl)-4-chloro- N-(3-fluoro-5-(phenylethynyl)pyridin- 2-yl)-1H-pyrazole-5-carboxamide | 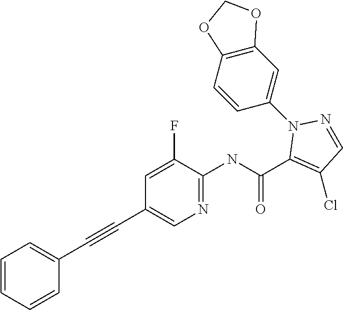 [SMILES: O=C(Nc1ncc(C#Cc2ccccc2)cc1F)c1c(Cl)cnn1-c1ccc2c(c1)OCO2] | 461.2 | 0.35 |
| 228 | 1-((1-acetyl-4-fluoropiperidin-4- yl)methyl)-4-chloro-N-(3-fluoro-5- (phenylethynyl)pyridin-2-yl)-1H- pyrazole-5-carboxamide | 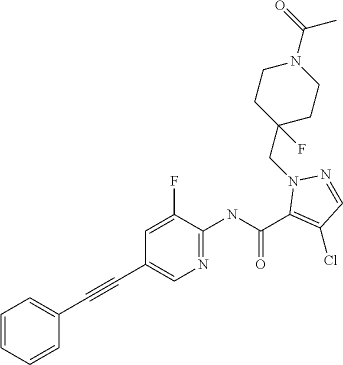 [SMILES: CC(=O)N1CCC(F)(Cn2ncc(Cl)c2C(=O)Nc2ncc(C#Cc3ccccc3)cc2F)CC1] | 498.2 | 0.35 |
| 229 | 4-chloro-1-(1- (cyclopropanecarbonyl)pyrrolidin-3- yl)-N-(5-((4-fluorophenyl)ethynyl)-3- methylpyridin-2-yl)-1H-pyrazole-5- carboxamide | 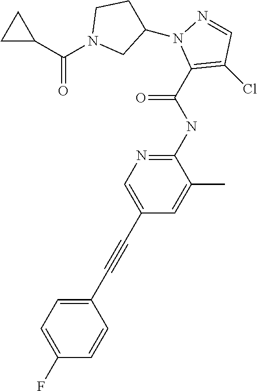 [SMILES: Cc1cc(C#Cc2ccc(F)cc2)cnc1NC(=O)c1c(Cl)cnn1C1CCN(C(=O)C2CC2)C1] | 492.3 | 0.35 |
| 230 | 1-(1-(1,4-dioxane-2- carbonyl)pyrrolidin-3-yl)-4-chloro-N- (5-((4-fluorophenyl)ethynyl)-3- methylpyridin-2-yl)-1H- pyrazole-5-carboxamide | 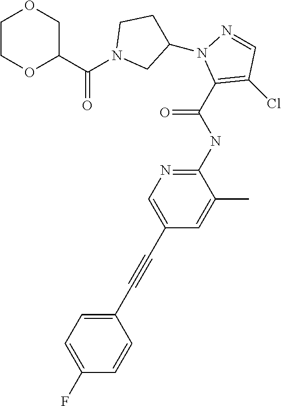 [SMILES: Cc1cc(C#Cc2ccc(F)cc2)cnc1NC(=O)c1c(Cl)cnn1C1CCN(C(=O)C2COCCO2)C1] | 538.2 | 0.35 |
| 231 | 4-chloro-1-((3,3-difluoro-1- isobutyrylpiperidin-4-yl)methyl)-N-(3- methyl-5-(phenylethynyl)pyridin-2- yl)-1H-pyrazole-5-carboxamide | 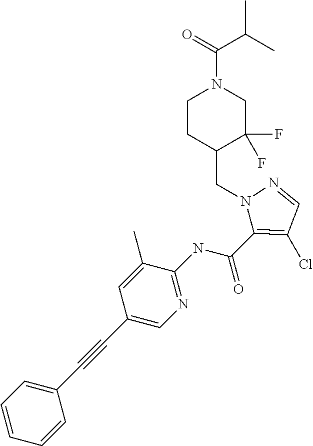 [SMILES: Cc1cc(C#Cc2ccccc2)cnc1NC(=O)c1c(Cl)cnn1CC1CCN(C(=O)C(C)C)CC1(F)F] | 540.2 | 0.35 |
| 232 | 4-chloro-N-(3-methyl-5- (phenylethynyl)pyridin-2-yl)-1- ((tetrahydrofuran-3-yl)methyl)-1H- pyrazole-5-carboxamide | 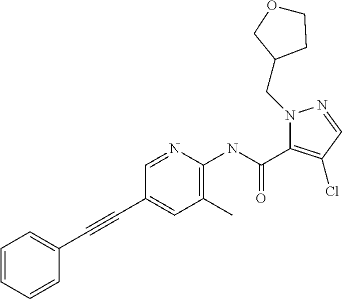 [SMILES: Cc1cc(C#Cc2ccccc2)cnc1NC(=O)c1c(Cl)cnn1CC1CCOC1] | 421 | 0.36 |
| 233 | 4-chloro-N-(3-fluoro-5-((4- fluorophenyl)ethynyl)pyridin-2-yl)-1- ((tetrahydro-2H-pyran-4-yl)methyl)- 1H-pyrazole-5-carboxamide | 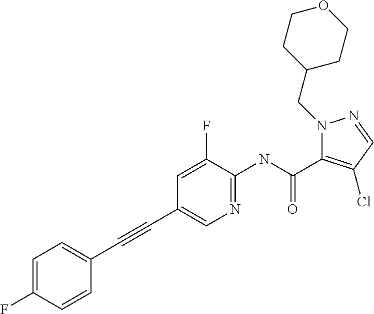 [SMILES: O=C(Nc1ncc(C#Cc2ccc(F)cc2)cc1F)c1c(Cl)cnn1CC1CCOCC1] | 457 | 0.36 |
| 234 | 4-chloro-1-(2-cyano-2-methylpropyl)- N-(3-fluoro-5-(phenylethynyl)pyridin- 2-yl)-1H-pyrazole-5-carboxamide | 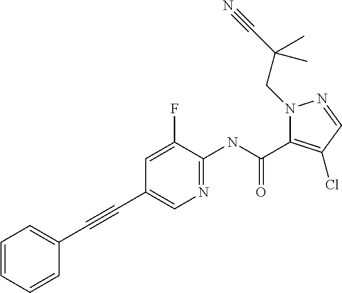 [SMILES: CC(C)(C#N)Cn1ncc(Cl)c1C(=O)Nc1ncc(C#Cc2ccccc2)cc1F] | 422 | 0.36 |
| 235 | 1-((3R,4R)-1-(1,4-dioxane-2- carbonyl)-3-fluoropiperidin-4-yl)-4- chloro-N-(3-fluoro-5-((4- fluorophenyl)ethynyl)pyridin-2-yl)- 1H-pyrazole-5-carboxamide | 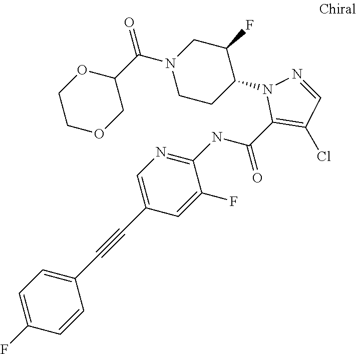 [SMILES: O=C(Nc1ncc(C#Cc2ccc(F)cc2)cc1F)c1c(Cl)cnn1[C@@H]1CCN(C(=O)C2COCCO2)C[C@H]1F] | 574 | 0.36 |
| 236 | 4-chloro-1-((4-fluoro-1- isobutyrylpiperidin-4-yl)methyl)-N-(3- methyl-5-(phenylethynyl)pyridin-2- yl)-1H-pyrazole-5-carboxamide | 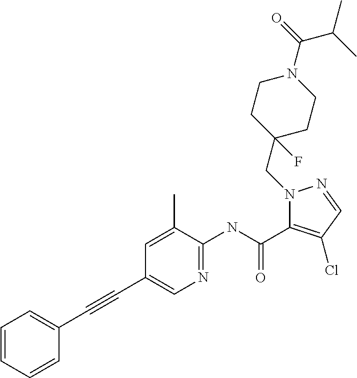 [SMILES: Cc1cc(C#Cc2ccccc2)cnc1NC(=O)c1c(Cl)cnn1CC1(F)CCN(C(=O)C(C)C)CC1] | 522.4 | 0.36 |
| 237 | tert-butyl 3-(4-chloro-5-((3-fluoro-5- (phenylethynyl)pyridin-2- yl)carbamoyl)-1H-pyrazol-1- yl)pyrrolidine-1-carboxylate | 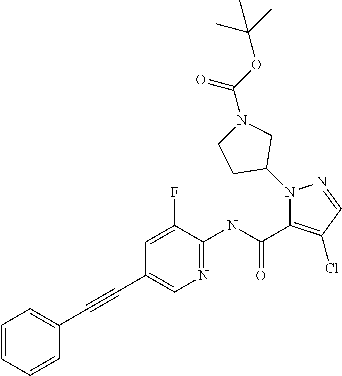 [SMILES: CC(C)(C)OC(=O)N1CCC(n2ncc(Cl)c2C(=O)Nc2ncc(C#Cc3ccccc3)cc2F)C1] | 454 | 0.37 |
| 238 | 1-(1-acetylpyrrolidin-3-yl)-4-chloro- N-(3-methyl-5- (phenylethynyl)pyridin-2-yl)-1H- pyrazole-5-carboxamide | 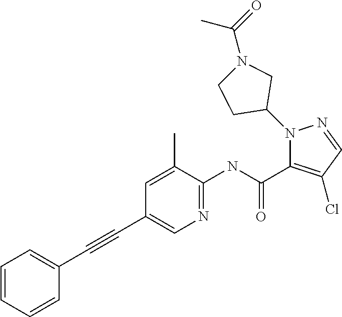 [SMILES: CC(=O)N1CCC(n2ncc(Cl)c2C(=O)Nc2ncc(C#Cc3ccccc3)cc2C)C1] | 448 | 0.37 |
| 239 | tert-butyl (2-(4-chloro-5-((3-methyl-5- (phenylethynyl)pyridin-2- yl)carbamoyl)-1H-pyrazol-1- yl)propyl)carbamate | 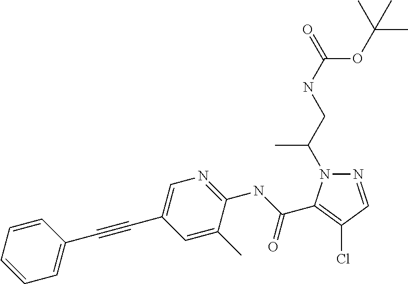 [SMILES: Cc1cc(C#Cc2ccccc2)cnc1NC(=O)c1c(Cl)cnn1C(C)CNC(=O)OC(C)(C)C] | 494.4 | 0.37 |
| 240 | tert-butyl (3S,4S)-4-((4-chloro-5-((3- fluoro-5-(phenylethynyl)pyridin-2- yl)carbamoyl)-1H-pyrazol-1- yl)methyl)-3-fluoropiperidine-1- carboxylate | 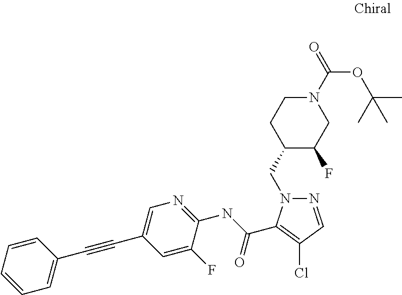 [SMILES: CC(C)(C)OC(=O)N1CC[C@@H](Cn2ncc(Cl)c2C(=O)Nc2ncc(C#Cc3ccccc3)cc2F)[C@H](F)C1] | 556.2 | 0.37 |
| 241 | tert-butyl (3S,4S)-4-((4-chloro-5-((3- methyl-5-(phenylethynyl)pyridin-2- yl)carbamoyl)-1H-pyrazol-1- yl)methyl)-3-fluoropiperidine-1- carboxylate | 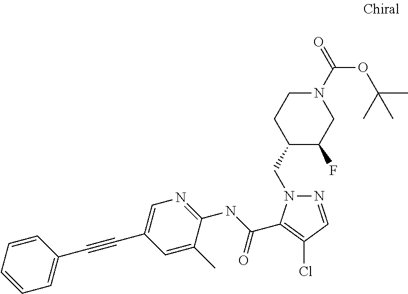 [SMILES: Cc1cc(C#Cc2ccccc2)cnc1NC(=O)c1c(Cl)cnn1C[C@@H]1CCN(C(=O)OC(C)(C)C)C[C@H]1F] | 552.3 | 0.37 |
| 242 | 4-chloro-1-((1- cyanocyclopentyl)methyl)-N-(3- methyl-5-(phenylethynyl)pyridin-2- yl)-1H-pyrazole-5-carboxamide | 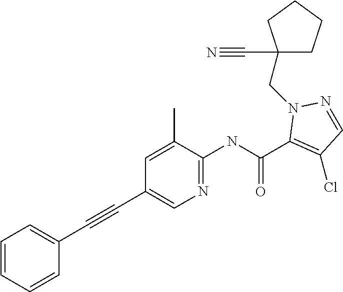 [SMILES: Cc1cc(C#Cc2ccccc2)cnc1NC(=O)c1c(Cl)cnn1CC1(C#N)CCCC1] | 444 | 0.38 |
| 243 | tert-butyl 3-(4-chloro-5-((3-fluoro-5- (phenylethynyl)pyridin-2- yl)carbamoyl)-1H-pyrazol-1- yl)azetidine-1-carboxylate | 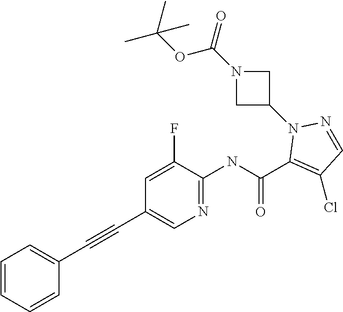 [SMILES: CC(C)(C)OC(=O)N1CC(n2ncc(Cl)c2C(=O)Nc2ncc(C#Cc3ccccc3)cc2F)C1] | 440 | 0.38 |
| 244 | 1-((1-acetyl-3-methylpiperidin-4- yl)methyl)-4-chloro-N-(5-((4- fluorophenyl)ethynyl)-3- methylpyridin-2-yl)-1H-pyrazole-5- carboxamide | 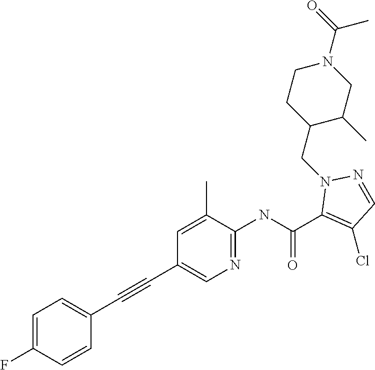 [SMILES: CC(=O)N1CCC(Cn2ncc(Cl)c2C(=O)Nc2ncc(C#Cc3ccc(F)cc3)cc2C)C(C)C1] | 508.3 | 0.38 |
| 245 | 4-chloro-1-(3-cyanocyclobutyl)-N- (2,6-difluoro-4- (phenylethynyl)phenyl)-1H-pyrazole- 5-carboxamide | 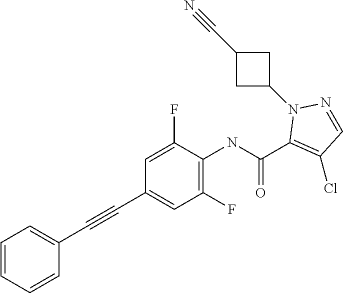 [SMILES: N#CC1CC(n2ncc(Cl)c2C(=O)Nc2c(F)cc(C#Cc3ccccc3)cc2F)C1] | 437 | 0.39 |
| 246 | 1-(2-(4-(1,4-dioxane-2- carbonyl)piperazin-1-yl)ethyl)-4- chloro-N-(5-((4- fluorophenyl)ethynyl)-3- methylpyridin-2-yl)-1H-pyrazole-5- carboxamide | 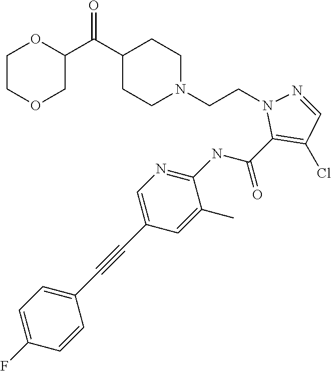 [SMILES: Cc1cc(C#Cc2ccc(F)cc2)cnc1NC(=O)c1c(Cl)cnn1CCN1CCC(C(=O)C2COCCO2)CC1] | 581.4 | 0.39 |
| 247 | 4-chloro-N-(3-fluoro-5-(thiophen-2- ylethynyl)pyridin-2-yl)-1-(1- pivaloylpiperidin-4-yl)-1H-pyrazole-5- carboxamide | 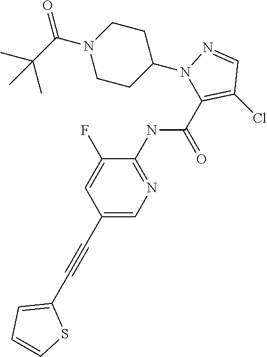 [SMILES: CC(C)(C)C(=O)N1CCC(n2ncc(Cl)c2C(=O)Nc2ncc(C#Cc3cccs3)cc2F)CC1] | 514.3 | 0.39 |
| 248 | 1-((1-acetylpiperidin-3-yl)methyl)-4- chloro-N-(5-((4- fluorophenyl)ethynyl)-3- methylpyridin-2-yl)-1H-pyrazole-5- carboxamide | 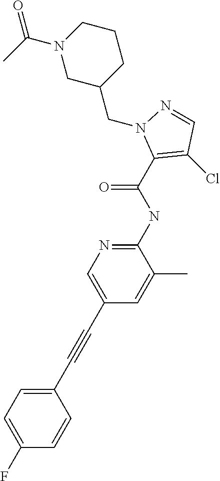 [SMILES: CC(=O)N1CCCC(Cn2ncc(Cl)c2C(=O)Nc2ncc(C#Cc3ccc(F)cc3)cc2C)C1] | 494.4 | 0.39 |
| 249 | 4-chloro-N-(5-((3- fluorophenyl)ethynyl)-3- methylpyridin-2-yl)-1-((1- isobutyrylpiperidin-4-yl)methyl)-1H- pyrazole-5-carboxamide | 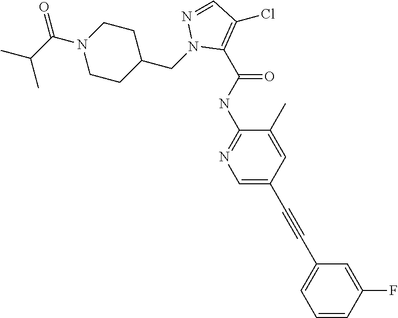 [SMILES: Cc1cc(C#Cc2cccc(F)c2)cnc1NC(=O)c1c(Cl)cnn1CC1CCN(C(=O)C(C)C)CC1] | 522 | 0.4 |
| 250 | 4-chloro-1-(2-(1- (cyclopropanecarbonyl)piperidin-4- yl)ethyl)-N-(3-fluoro-5-(thiophen-2- ylethynyl)pyridin-2-yl)-1H-pyrazole-5- carboxamide | 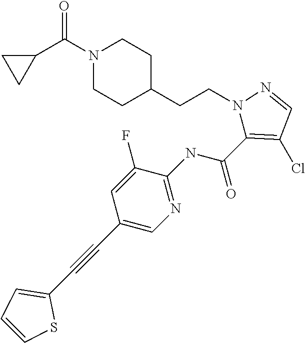 [SMILES: O=C(Nc1ncc(C#Cc2cccs2)cc1F)c1c(Cl)cnn1CCC1CCN(C(=O)C2CC2)CC1] | 526.2 | 0.4 |
| 251 | 1-((1-(1,4-dioxane-2-carbonyl)-4- methylpiperidin-4-yl)methyl)-4- chloro-N-(3-methyl-5- (phenylethynyl)pyridin-2-yl)-1H- pyrazole-5-carboxamide | 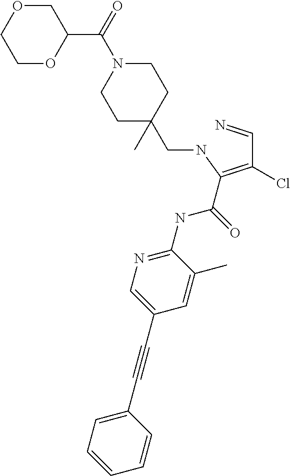 [SMILES: Cc1cc(C#Cc2ccccc2)cnc1NC(=O)/C(NCC1(C)CCN(C(=O)C2COCCO2)CC1)=C(\Cl)C=N] | 562.4 | 0.4 |
| 252 | (S)-1-((1,4-dioxan-2-yl)methyl)-4- chloro-N-(2,6-difluoro-4- (phenylethynyl)phenyl)-1H-pyrazole- 5-carboxamide | 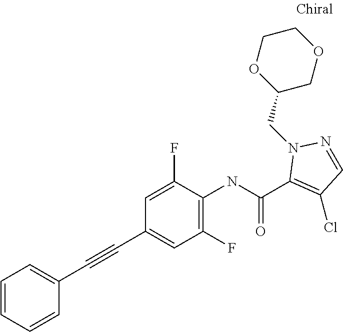 [SMILES: O=C(Nc1c(F)cc(C#Cc2ccccc2)cc1F)c1c(Cl)cnn1C[C@H]1COCCO1] | 458 | 0.41 |
| 253 | 4-chloro-1-(3-cyanopropyl)-N-(2,6- difluoro-4-(phenylethynyl)phenyl)- 1H-pyrazole-5-carboxamide | 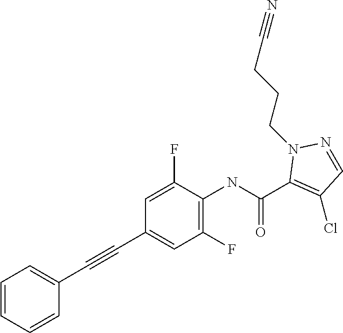 [SMILES: N#CCCCn1ncc(Cl)c1C(=O)Nc1c(F)cc(C#Cc2ccccc2)cc1F] | 425 | 0.41 |
| 254 | 4-chloro-1-(3-cyanocyclobutyl)-N-(3- methyl-5-(phenylethynyl)pyridin-2- yl)-1H-pyrazole-5-carboxamide | 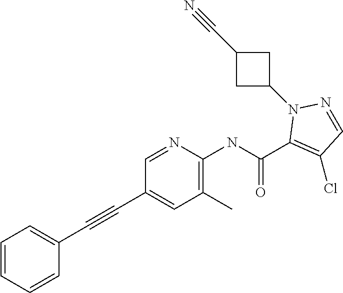 [SMILES: Cc1cc(C#Cc2ccccc2)cnc1NC(=O)c1c(Cl)cnn1C1CC(C#N)C1] | 416 | 0.42 |
| 255 | tert-butyl 4-(4-chloro-5-((3-fluoro-5- (phenylethynyl)pyridin-2- yl)carbamoyl)-1H-pyrazol-1- yl)piperidine-1-carboxylate | 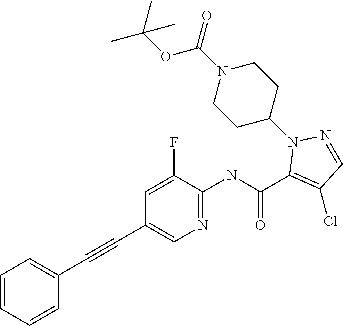 [SMILES: CC(C)(C)OC(=O)N1CCC(n2ncc(Cl)c2C(=O)Nc2ncc(C#Cc3ccccc3)cc2F)CC1] | 546 (M + Na) | 0.42 |
| 256 | 1-((1-acetylpiperidin-4-yl)methyl)-4- chloro-N-(5-((4- fluorophenyl)ethynyl)-3- methylpyridin-2-yl)-1H-pyrazole-5- carboxamide | 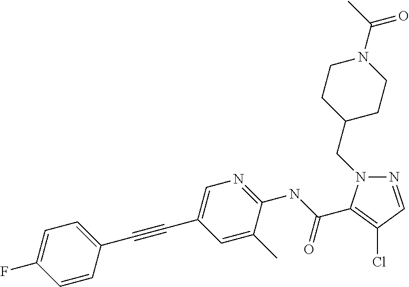 [SMILES: CC(=O)N1CCC(Cn2ncc(Cl)c2C(=O)Nc2ncc(C#Cc3ccc(F)cc3)cc2C)CC1] | 494.4 | 0.42 |
| 257 | tert-butyl 4-(2-(4-chloro-5-((3-fluoro- 5-(phenylethynyl)pyridin-2- yl)carbamoyl)-1H-pyrazol-1- yl)ethyl)piperazine-1-carboxylate | 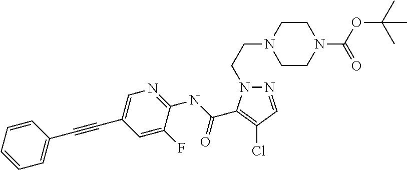 [SMILES: CC(C)(C)OC(=O)N1CCN(CCn2ncc(Cl)c2C(=O)Nc2ncc(C#Cc3ccccc3)cc2F)CC1] | 553.4 | 0.42 |
| 258 | 4-chloro-N-(5-((4- fluorophenyl)ethynyl)-3- methylpyridin-2-yl)-1-(1-(3- methylbutanoyl)pyrrolidin-3-yl)-1H- pyrazole-5-carboxamide | 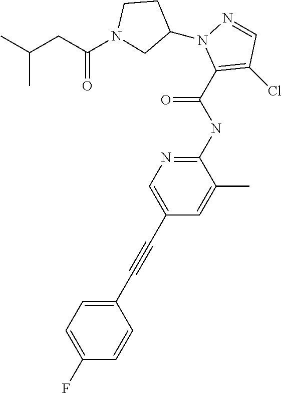 [SMILES: Cc1cc(C#Cc2ccc(F)cc2)cnc1NC(=O)c1c(Cl)cnn1C1CCN(C(=O)CC(C)C)C1] | 508.2 | 0.42 |
| 259 | 4-chloro-N-[3-methyl-5- (phenylethynyl)pyridin-2-yl]-1- {[(1S,2S,4R)-7- oxabicyclo[2.2.1]heptan-2-yl]methyl}- 1H-pyrazole-5-carboxamide and 4-chloro-N-[3-methyl-5- (phenylethynyl)pyridin-2-yl]-1- {[(1R,2R,4S)-7- oxabicyclo[2.2.1]heptan-2-yl]methyl}- 1H-pyrazole-5-carboxamide | 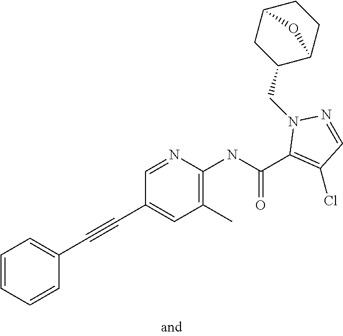 [SMILES: Cc1cc(C#Cc2ccccc2)cnc1NC(=O)c1c(Cl)cnn1C[C@@H]1C[C@H]2CC[C@@H]1O2] | 447 | 0.43 |
| | | 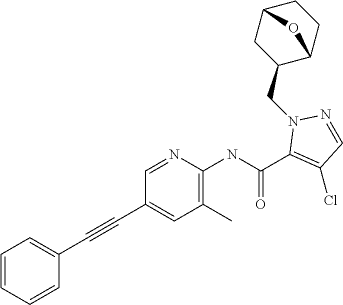 [SMILES: Cc1cc(C#Cc2ccccc2)cnc1NC(=O)c1c(Cl)cnn1C[C@H]1C[C@@H]2CC[C@H]1O2] | | |
| 260 | 1-[(3aR,5s,6aS)-2- acetyloctahydrocyclopenta[c]pyrrol-5- yl]-4-chloro-N-[3-methyl-5- (phenylethynyl)pyridin-2-yl]-1H- pyrazole-5-carboxamide | 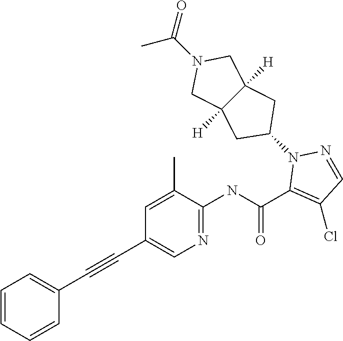 [SMILES: CC(=O)N1C[C@H]2C[C@H](n3ncc(Cl)c3C(=O)Nc3ncc(C#Cc4ccccc4)cc3C)C[C@H]2C1] | 488 | 0.26 |
| 261*⁶ | 1-[(2S,4R)-1-acetyl-2- methylpiperidin-4-yl]-4-chloro-N-[3- methyl-5-(phenylethynyl)pyridin-2- yl]-1H-pyrazole-5-carboxamide or 1-[(2R,4S)-1-acetyl-2- methylpiperidin-4-yl]-4-chloro-N-[3- methyl-5-(phenylethynyl)pyridin-2- yl]-1H-pyrazole-5-carboxamide | 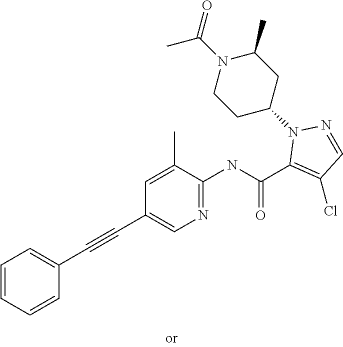 [SMILES: CC(=O)N1CC[C@@H](n2ncc(Cl)c2C(=O)Nc2ncc(C#Cc3ccccc3)cc2C)C[C@@H]1C] | 476.3 | 0.43 |
| | | 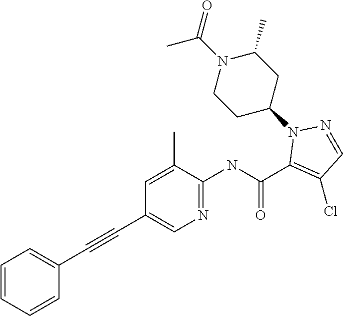 [SMILES: CC(=O)N1CC[C@H](n2ncc(Cl)c2C(=O)Nc2ncc(C#Cc3ccccc3)cc2C)C[C@H]1C] | | |
| 262 | (S)-1-((1-acetylpiperidin-3-yl)methyl)- 4-chloro-N-(3-fluoro-5- (phenylethynyl)pyridin-2-yl)-1H- pyrazole-5-carboxamide | 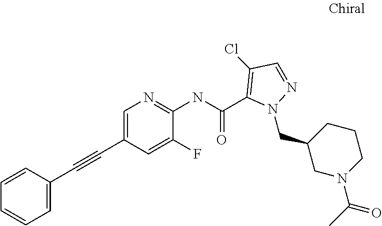 [SMILES: CC(=O)N1CCC[C@H](Cn2ncc(Cl)c2C(=O)Nc2ncc(C#Cc3ccccc3)cc2F)C1] | 480.3 | 0.43 |
| 263 | (S)-4-chloro-1-((1-isobutyrylpiperidin- 3-yl)methyl)-N-(3-methyl-5- (phenylmethyl)pyridin-2-yl)-1H- pyrazole-5-carboxamide | 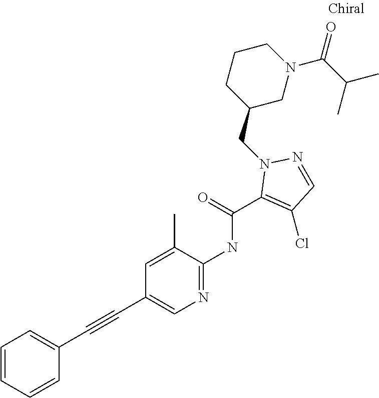 [SMILES: Cc1cc(C#Cc2ccccc2)cnc1NC(=O)c1c(Cl)cnn1C[C@H]1CCCN(C(=O)C(C)C)C1] | 504.3 | 0.43 |
| 264 | 4-chloro-N-(5-((4- fluorophenyl)ethynyl)-3- methylpyridin-2-yl)-1-((1-(2- methylcyclopropane-1- carbonyl)piperidin-3-yl)methyl)-1H- pyrazole-5-carboxamide | 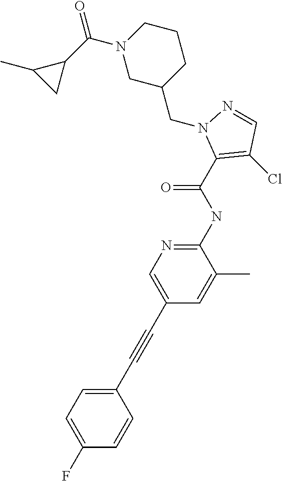 [SMILES: Cc1cc(C#Cc2ccc(F)cc2)cnc1NC(=O)c1c(Cl)cnn1CC1CCCN(C(=O)C2CC2C)C1] | 534.4 | 0.43 |
| 265 | tert-butyl 4-((4-chloro-5-((3-methyl-5- (phenylethynyl)pyridin-2- yl)carbamoyl)-1H-pyrazol-1- yl)methyl)-3-methylpiperidine-1- carboxylate | 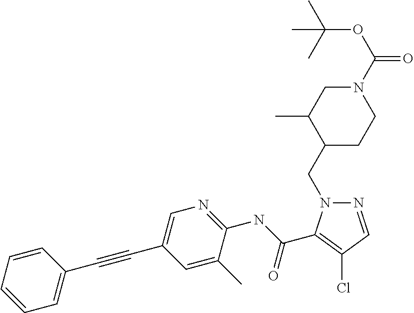 [SMILES: Cc1cc(C#Cc2ccccc2)cnc1NC(=O)c1c(Cl)cnn1CC1CCN(C(=O)OC(C)(C)C)CC1C] | 548.2 | 0.43 |
| 266 | 4-chloro-1-(3-cyanocyclobutyl)-N-(3- fluoro-3-(phenylethynyl)pyridin-2-yl)- 1H-pyrazole-5-carboxamide | 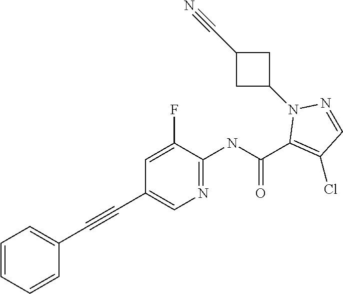 [SMILES: N#CC1CC(n2ncc(Cl)c2C(=O)Nc2ncc(C#Cc3ccccc3)cc2F)C1] | 420 | 0.44 |
| 267 | 4-chloro-1-((1- cyanocyclopropyl)methyl)-N-(3- methyl-5-(phenylethynyl)pyridin-2- yl)-1H-pyrazole-5-carboxamide | 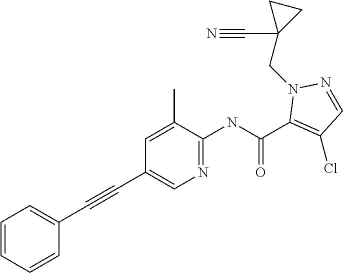 [SMILES: Cc1cc(C#Cc2ccccc2)cnc1NC(=O)c1c(Cl)cnn1CC1(C#N)CC1] | 416 | 0.45 |
| 268 | 4-chloro-N-(3-methyl-5- (phenylethynyl)pyridin-2-yl)-1-(1- (2,2,2-trifluoroacetyl)piperidin-4-yl)- 1H-pyrazole-5-carboxamide | 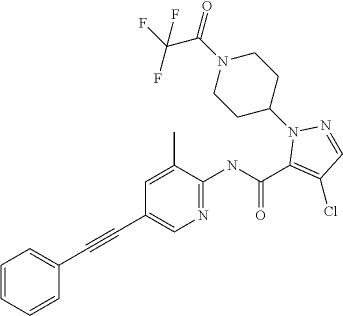 [SMILES: Cc1cc(C#Cc2ccccc2)cnc1NC(=O)c1c(Cl)cnn1C1CCN(C(=O)C(F)(F)F)CC1] | 516.3 | 0.45 |
| 269 | tert-butyl 4-((4-chloro-5-((5-((4- fluorophenyl)ethynyl)-3- methylpyridin-2-yl)carbamoyl)-1H- pyrazol-1-yl)methyl)-4- cyanopiperidine-1-carboxylate | 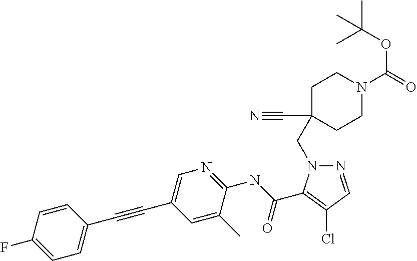 [SMILES: Cc1cc(C#Cc2ccc(F)cc2)cnc1NC(=O)c1c(Cl)cnn1CC1(C#N)CCN(C(=O)OC(C)(C)C)CC1] | 577.3 | 0.45 |
| 270 | 4-chloro-N-(3-fluoro-5- (phenylethynyl)pyridin-2-yl)-1-(1- methyl-1H-indazole-5-yl)-1H-pyrazole- 5-carboxamide | 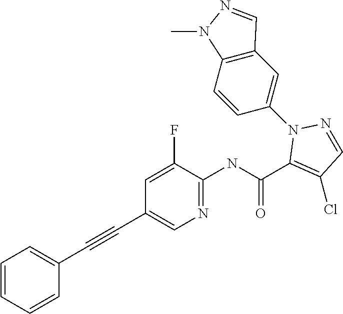 [SMILES: Cn1ncc2cc(-n3ncc(Cl)c3C(=O)Nc3ncc(C#Cc4ccccc4)cc3F)ccc21] | 471 | 0.46 |
| 271 | 4-chloro-1-((1- (cyclopropanecarbonyl)piperidin-3- yl)methyl)-N-(5-((4- fluorophenyl)ethynyl)-3- methylpyridin-2-yl)-1H-pyrazole-5- carboxamide | 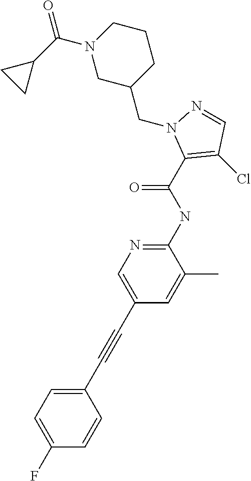 [SMILES: Cc1cc(C#Cc2ccc(F)cc2)cnc1NC(=O)c1c(Cl)cnn1CC1CCCN(C(=O)C2CC2)C1] | 520.4 | 0.46 |
| 272 | 4-chloro-N-(5-((4- fluorophenyl)ethynyl)-3- methylpyridin-2-yl)-1-((1- isobutyrylpiperidin-3-yl) methyl)-1H- pyrazole-5-carboxamide | 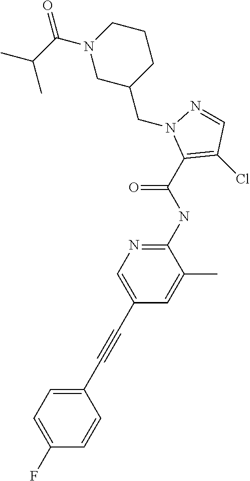 [SMILES: Cc1cc(C#Cc2ccc(F)cc2)cnc1NC(=O)c1c(Cl)cnn1CC1CCCN(C(=O)C(C)C)C1] | 522.4 | 0.46 |
| 273 | 4-chloro-1-[(3aR,5s,6aS)-2- (cyclopropanecarbonyl)octahydro- cyclopenta[c]pyrrol-5-yl]-N-{5-[(3- fluorophenyl)ethynyl]-3- methylpyridin-2-yl}-1H-pyrazole-3- carboxamide | 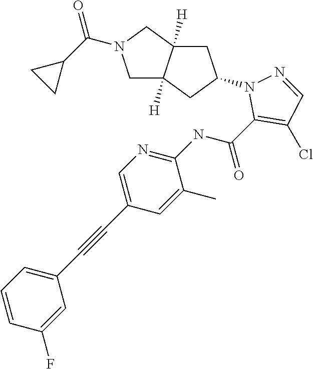 [SMILES: Cc1cc(C#Cc2cccc(F)c2)cnc1NC(=O)c1c(Cl)cnn1[C@H]1C[C@@H]2CN(C(=O)C3CC3)C[C@@H]2C1] | 532.3 | 0.47 |
| 274 | 4-chloro-1-((1- (cyclopropanecarbonyl)azetidin-3- yl)methyl)-N-(3-methyl-5- (phenylethynyl)pyridin-2-yl)-1H- pyrazole-5-carboxamide | 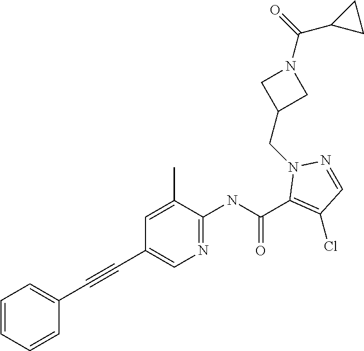 [SMILES: Cc1cc(C#Cc2ccccc2)cnc1NC(=O)c1c(Cl)cnn1CC1CN(C(=O)C2CC2)C1] | 474.2 | 0.47 |
| 275 | (S)-4-chloro-N-(3-fluoro-5- (phenylethynyl)pyridin-2-yl)-1- (tetrahydrofuran-3-yl)-1H-pyrazole-5- carboxamide | 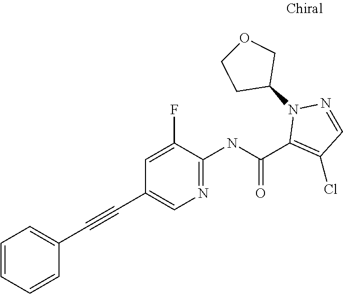 [SMILES: O=C(Nc1ncc(C#Cc2ccccc2)cc1F)c1c(Cl)cnn1[C@H]1CCOC1] | 411 | 0.48 |
| 276 | 4-chloro-1-(2-cyano-2-methylpropyl)- N-(3-methyl-5- (phenylethynyl)pyridin-2-yl)-1H- pyrazole-5-carboxamide | 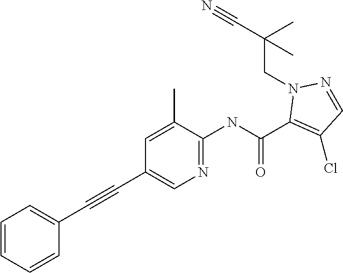 [SMILES: Cc1cc(C#Cc2ccccc2)cnc1NC(=O)c1c(Cl)cnn1CC(C)(C)C#N] | 418 | 0.48 |
| 277 | tert-butyl 4-(4-chloro-5-((3-methyl-5- (phenylethynyl)pyridin-2- yl)carbamoyl)-1H-pyrazol-1- yl)azepane-1-carboxylate | 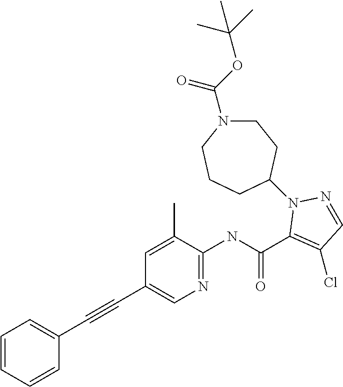 [SMILES: Cc1cc(C#Cc2ccccc2)cnc1NC(=O)c1c(Cl)cnn1C1CCCN(C(=O)OC(C)(C)C)CC1] | 534.4 | 0.48 |
| 278 | 4-chloro-1-(cyclopropylmethyl)-N-(3- methyl-5-(phenylethynyl)pyridin-2- yl)-1H-pyrazole-5-carboxamide | 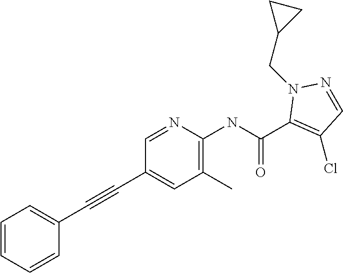 [SMILES: Cc1cc(C#Cc2ccccc2)cnc1NC(=O)c1c(Cl)cnn1CC1CC1] | 391 | 0.49 |
| 279 | 4-chloro-N-(3-fluoro-5- (phenylethynyl)pyridin-2-yl)-1-((2- methyltetrahydrofuran-2-yl)methyl)- 1H-pyrazole-5-carboxamide | 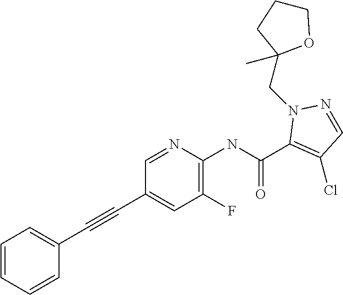 [SMILES: CC1(Cn2ncc(Cl)c2C(=O)Nc2ncc(C#Cc3ccccc3)cc2F)CCCO1] | 439 | 0.49 |
| 280 | 4-chloro-N-(3-methyl-5- (phenylethynyl)pyridin-2-yl)-1- ((tetrahydro-2H-pyran-3-yl)methyl)- 1H-pyrazole-5-carboxamide | 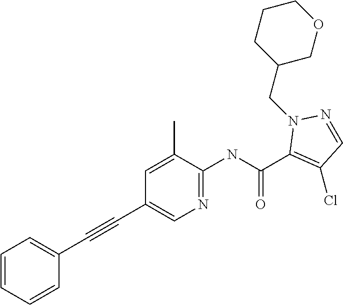 [SMILES: Cc1cc(C#Cc2ccccc2)cnc1NC(=O)c1c(Cl)cnn1CC1CCCOC1] | 435 | 0.49 |
| 281 | 4-chloro-N-(3-methyl-5- (phenylethynyl)pyridin-2-yl)-1-((4- methyltetrahydrofuran-3-yl)methyl)- 1H-pyrazole-5-carboxamide | 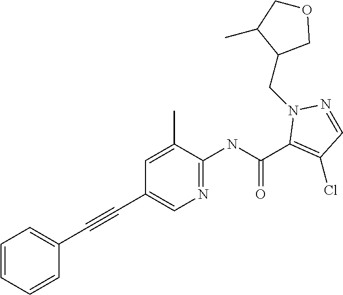 [SMILES: Cc1cc(C#Cc2ccccc2)cnc1NC(=O)c1c(Cl)cnn1CC1COCC1C] | 435 | 0.49 |
| 282 | 4-chloro-1-[(3aR,5s,6aS)-2- (cyclopropanecarbonyl)octahydro- cyclopenta[c]pyrrol-5-yl]-N-[3-methyl-5- (phenylethynyl)pyridin-2-yl]-1H- pyrazole-5-carboxamide | 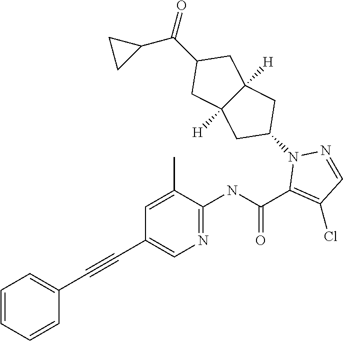 [SMILES: Cc1cc(C#Cc2ccccc2)cnc1NC(=O)c1c(Cl)cnn1[C@@H]1C[C@H]2CC(C(=O)C3CC3)C[C@H]2C1] | 514 | 0.49 |
| 283 | 1-(benzo[d][1,3]dioxol-5-yl)-4-chloro- N-(3-methyl-5- (phenylethynyl)pyridin-2-yl)-1H- pyrazole-5-carboxamide | 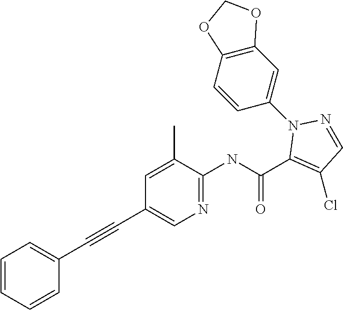 [SMILES: Cc1cc(C#Cc2ccccc2)cnc1NC(=O)c1c(Cl)cnn1-c1ccc2c(c1)OCO2] | 457 | 0.49 |
| 284 | 4-chloro-N-(5-((4- fluorophenyl)ethynyl)-3- methylpyridin-2-yl)-1-((1-isobutyryl- 3-methylpiperidin-4-yl)methyl)-1H- pyrazole-5-carboxamide | 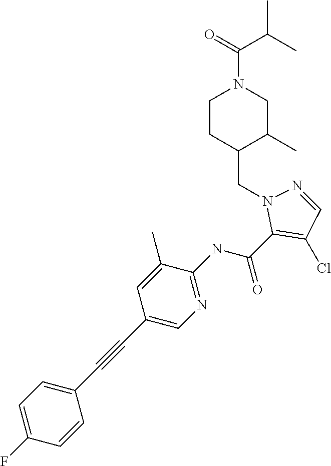 [SMILES: Cc1cc(C#Cc2ccc(F)cc2)cnc1NC(=O)c1c(Cl)cnn1CC1CCN(C(=O)C(C)C)CC1C] | 536.2 | 0.49 |
| 285 | (S)-1-((1,4-dioxan-2-yl)methyl)-4- chloro-N-(2-fluoro-6-methyl-4- (phenylethynyl)phenyl)-1H- pyrazole-5-carboxamide | 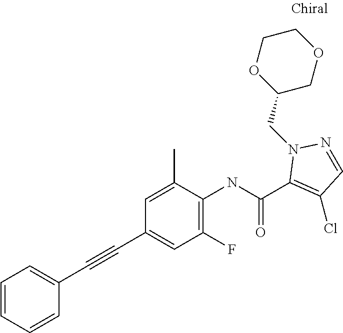 [SMILES: Cc1cc(C#Cc2ccccc2)cc(F)c1NC(=O)c1c(Cl)cnn1C[C@H]1COCCO1] | 454 | 0.5 |
| 286 | (S)-1-((1,4-dioxan-2-yl)methyl)-4- chloro-N-(5-((3- fluorophenyl)ethynyl)-3- methylpyridin-2-yl)-1H-pyrazole-5- carboxamide | 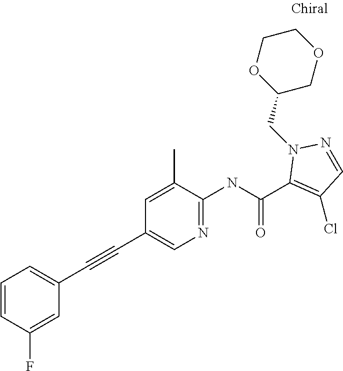 [SMILES: Cc1cc(C#Cc2cccc(F)c2)cnc1NC(=O)c1c(Cl)cnn1C[C@H]1COCCO1] | 455 | 0.5 |
| 287 | tert-butyl 3-(4-chloro-5-((3-methyl-5- (phenylethynyl)pyridin-2- yl)carbamoyl)-1H-pyrazol-1- yl)azetidine-1-carboxylate | 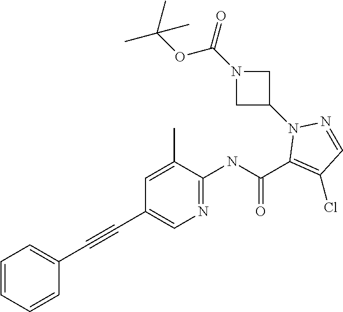 [SMILES: Cc1cc(C#Cc2ccccc2)cnc1NC(=O)c1c(Cl)cnn1C1CN(C(=O)OC(C)(C)C)C1] | 492 | 0.5 |
| 288 | (S)-4-chloro-N-(3-fluoro-5- (phenylethynyl)pyridin-2-yl)-1-((1- isobutyrylpiperidin-3-yl)methyl)-1H- pyrazole-5-carboxamide | 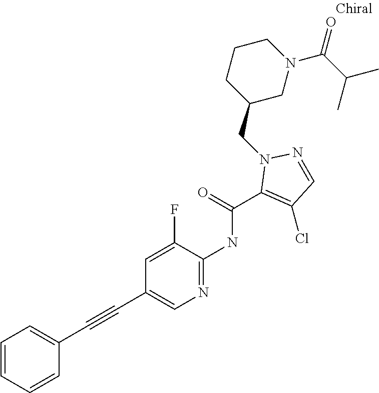 [SMILES: CC(C)C(=O)N1CCC[C@H](Cn2ncc(Cl)c2C(=O)Nc2ncc(C#Cc3ccccc3)cc2F)C1] | 508.2 | 0.5 |
| 289 | 4-chloro-1-(3-cyanocyclopentyl)-N-(3- methyl-5-(phenylethynyl)pyridin-2- yl)-1H-pyrazole-5-carboxamide | 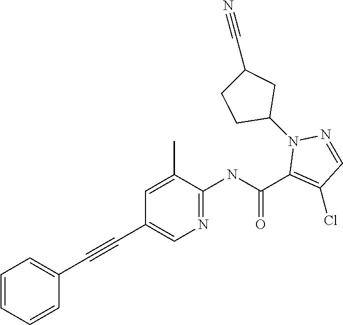 [SMILES: Cc1cc(C#Cc2ccccc2)cnc1NC(=O)c1c(Cl)cnn1C1CCC(C#N)C1] | 430 | 0.51 |
| 290 | tert-butyl 4-(2-(4-chloro-5-((3-fluoro- 5-(phenylethynyl)pyridin-2- yl)carbamoyl)-1H-pyrazol-1- yl)ethyl)piperidine-1-carboxylate | 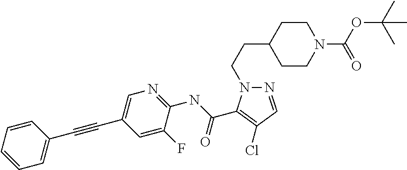 [SMILES: CC(C)(C)OC(=O)N1CCC(CCn2ncc(Cl)c2C(=O)Nc2ncc(C#Cc3ccccc3)cc2F)CC1] | 552.3 | 0.51 |
| 291 | tert-butyl ((1R,3R)-3-(4-chloro-5-((3- methyl-5-(phenylethynyl)pyridin-2- yl)carbamoyl)-1H-pyrazol-1- yl)cyclopentyl)carbamate | 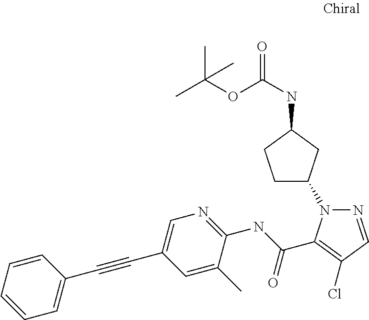 [SMILES: Cc1cc(C#Cc2ccccc2)cnc1NC(=O)c1c(Cl)cnn1[C@@H]1CC[C@@H](NC(=O)OC(C)(C)C)C1] | 520.3 | 0.52 |
| 292 | tert-butyl (trans-3-(4-chloro-5-((3- methyl-5-(phenylethynyl)pyridin-2- yl)carbamoyl)-1H-pyrazol-1- yl)cyclobutyl)carbamate | 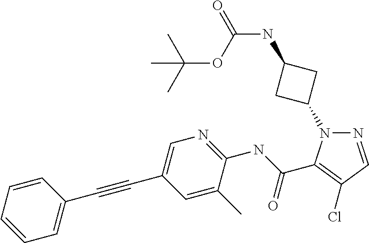 [SMILES: Cc1cc(C#Cc2ccccc2)cnc1NC(=O)c1c(Cl)cnn1[C@H]1C[C@H](NC(=O)OC(C)(C)C)C1] | 506.4 | 0.52 |
| 293 | 4-chloro-1-(((3S,4S)-3-fluoro-1- isobutyrylpiperidin-4-yl)methyl)-N-(5- ((4-fluorophenyl)ethynyl)-3- methylpyridin-2-yl)-1H- pyrazole-5-carboxamide | 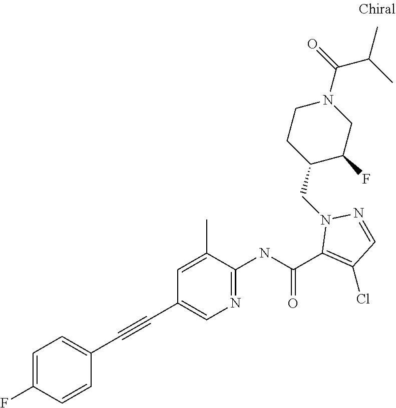 [SMILES: Cc1cc(C#Cc2ccc(F)cc2)cnc1NC(=O)c1c(Cl)cnn1C[C@@H]1CCN(C(=O)C(C)C)C[C@H]1F] | 540.2 | 0.52 |
| 294 | tert-butyl (cis-4-(4-chloro-5-((3- fluoro-5-(phenylethynyl)pyridin-2- yl)carbamoyl)-1H-pyrazol-1- yl)cyclohexyl)carbamate | 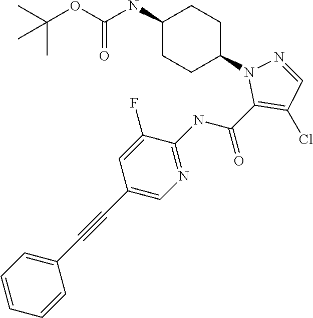 [SMILES: CC(C)(C)OC(=O)N[C@H]1CC[C@@H](n2ncc(Cl)c2C(=O)Nc2ncc(C#Cc3ccccc3)cc2F)CC1] | 538 | 0.53 |
| 295 | 4-chloro-1-((1- (cyclopropanecarbonyl)-4- methylpyridin-4-yl)methyl)-N-(3- methyl-5-(phenylethynyl)pyridin-2- yl)-1H-pyrazole-5-carboxamide | 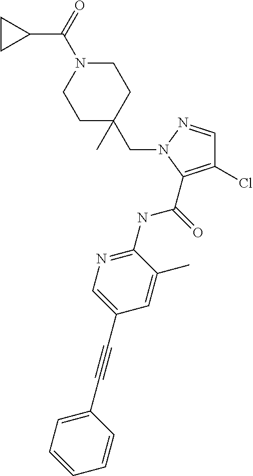 [SMILES: Cc1cc(C#Cc2ccccc2)cnc1NC(=O)c1c(Cl)cnn1CC1(C)CCN(C(=O)C2CC2)CC1] | 516.4 | 0.53 |
| 296 | (S)-1-((1-acetylpiperidin-3-yl)methyl)- 4-chloro-N-(3-methyl-5- (phenylethynyl)pyridin-2-yl)-1H- pyrazole-5-carboxamide | 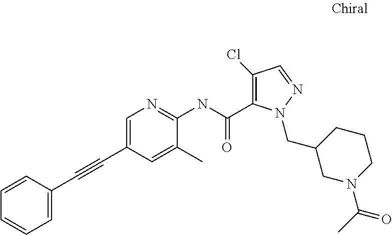 [SMILES: CC(=O)N1CCCC(Cn2ncc(Cl)c2C(=O)Nc2ncc(C#Cc3ccccc3)cc2C)C1] | 476.3 | 0.53 |
| 297 | tert-butyl 3-(4-chloro-5-((2,6-difluoro- 4-(phenyethynyl)phenyl)carbamoyl)- 1H-pyrazol-1-yl)pyrroldine-1- carboxylate | 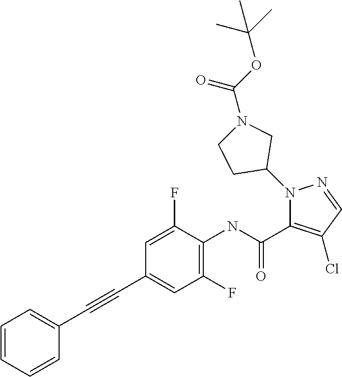 [SMILES: CC(C)(C)OC(=O)N1CCC(n2ncc(Cl)c2C(=O)Nc2c(F)cc(C#Cc3ccccc3)cc2F)C1] | 549 | 0.54 |
| 298 | tert-butyl (S)-3-(4-chloro-5-((3- methyl-5-(phenylethynyl)pyridin-2- yl)carbamoyl)-1H-pyrazol-1- yl)pyrrolidine-1-carboxylate | 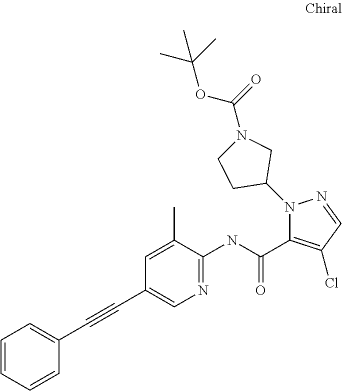 [SMILES: Cc1cc(C#Cc2ccccc2)cnc1NC(=O)c1c(Cl)cnn1C1CCN(C(=O)OC(C)(C)C)C1] | 506 | 0.54 |
| 299 | 4-chloro-1-(1- (cyclopentanecarbonyl)piperidin-4-yl)- N-(3-fluoro-5-(phenylethynyl)pyridin- 2-yl)-1H-pyrazole-5-carboxamide | 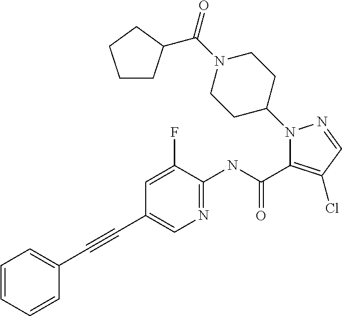 [SMILES: O=C(Nc1ncc(C#Cc2ccccc2)cc1F)c1c(Cl)cnn1C1CCN(C(=O)C2CCCC2)CC1] | 520.3 | 0.54 |
| 300 | tert-butyl 2-((4-chloro-5-((3-fluoro-5- (phenylethynyl)pyridin-2- yl)carbamoyl)-1H-pyrazol-1- yl)methyl)morpholine-4-carboxylate | 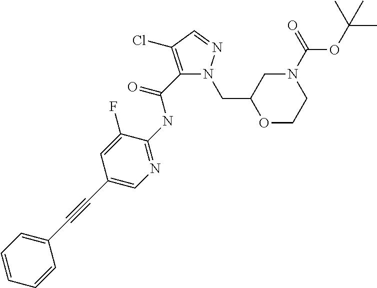 [SMILES: CC(C)(C)OC(=O)N1CCOC(Cn2ncc(Cl)c2C(=O)Nc2ncc(C#Cc3ccccc3)cc2F)C1] | 540.4 | 0.54 |
| 301 | tert-butyl 4-((4-chloro-5-((3-methyl-5- (phenylethynyl)pyridin-2- yl)carbamoyl)-1H-pyrazol-1- yl)methyl)-4-methylpiperidine-1- carboxylate | 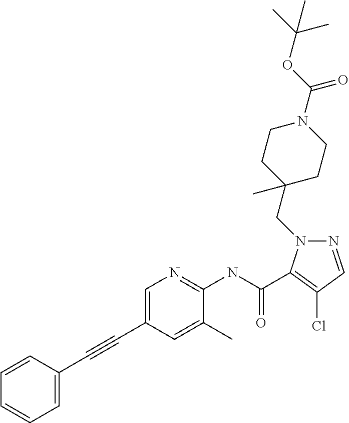 [SMILES: Cc1cc(C#Cc2ccccc2)cnc1NC(=O)c1c(Cl)cnn1CC1(C)CCN(C(=O)OC(C)(C)C)CC1] | 548.3 | 0.54 |
| 302 | 4-chloro-1-((1- (cyclopropanecarbonyl)-4- fluoropiperidin-4-yl)methyl)-N-(5-((4- fluorophenyl)ethynyl)-3- methylpyridin-2-yl)-1H-pyrazole-5- carboxamide | 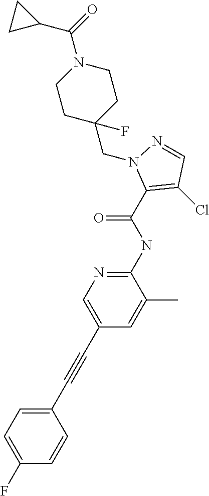 [SMILES: Cc1cc(C#Cc2ccc(F)cc2)cnc1NC(=O)c1c(Cl)cnn1CC1(F)CCN(C(=O)C2CC2)CC1] | 538.2 | 0.55 |
| 303 | 1-(2-(4-acetylpiperazin-1-yl)ethyl)-4- chloro-N-(3-methyl-5- (phenylethynyl)pyridin-2-yl)-1H- pyrazole-5-carboxamide | 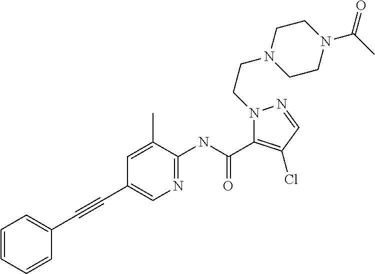 [SMILES: CC(=O)N1CCN(CCn2ncc(Cl)c2C(=O)Nc2ncc(C#Cc3ccccc3)cc2C)CC1] | 491.3 | 0.56 |
| 304 | 1-(benzofuran-5-yl)-4-chloro-N-(3- methyl-5-(phenylethynyl)pyridin-2- yl)-1H-pyrazole-5-carboxamide | 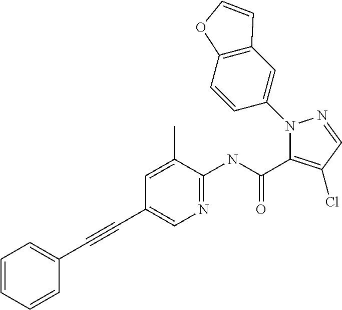 [SMILES: Cc1cc(C#Cc2ccccc2)cnc1NC(=O)c1c(Cl)cnn1-c1ccc2occc2c1] | 453.3 | 0.57 |
| 305 | 4-chloro-N-(5-((4- fluorophenyl)ethynyl)-3- methylpyridin-2-yl)-1-(1- isobutyrylpyrrolidin-3-yl)-1H- pyrazole-5-carboxamide | 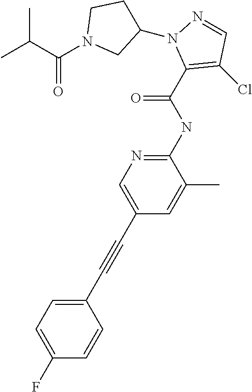 [SMILES: Cc1cc(C#Cc2ccc(F)cc2)cnc1NC(=O)c1c(Cl)cnn1C1CCN(C(=O)C(C)C)C1] | 494.4 | 0.57 |
| 306 | 4-chloro-N-(3-methyl-5- (phenylethynyl)pyridin-2-yl)-1- (tetrahydrofuran-3-yl)-1H-pyrazole-5- carboxamide | 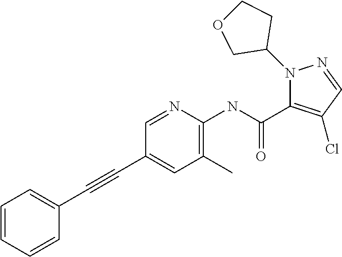 [SMILES: Cc1cc(C#Cc2ccccc2)cnc1NC(=O)c1c(Cl)cnn1C1CCOC1] | 407 | 0.58 |
| 307 | tert-butyl 3-(4-chloro-5-((3-methyl-5- (phenylethynyl)pyridin-2- yl)carbamoyl)-1H-pyrazol-1- yl)pyrrolidine-1-carboxylate | 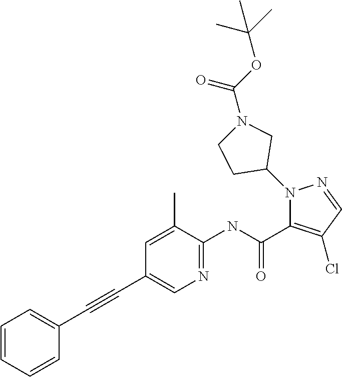 [SMILES: Cc1cc(C#Cc2ccccc2)cnc1NC(=O)c1c(Cl)cnn1C1CCN(C(=O)OC(C)(C)C)C1] | 506 | 0.58 |
| 308 | tert-butyl 4-(2-(4-chloro-5-((3-methyl- 5-(phenylethynyl)pyridin-2- yl)carbamoyl)-1H-pyrazol-1- yl)ethyl)piperidine-1-carboxylate | 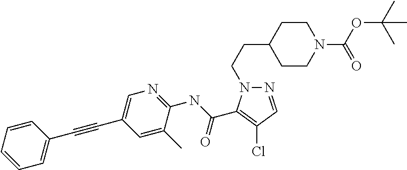 [SMILES: Cc1cc(C#Cc2ccccc2)cnc1NC(=O)c1c(Cl)cnn1CCC1CCN(C(=O)OC(C)(C)C)CC1] | 548 | 0.58 |
| 309 | tert-butyl (cis-4-(4-chloro-5-((3- methyl-5-(phenylethynyl)pyridin-2- yl)carbamoyl)-1H-pyrazol-1- yl)cyclohexyl)carbamate hydrofluoride | 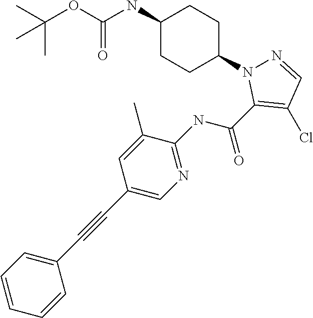 [SMILES: Cc1cc(C#Cc2ccccc2)cnc1NC(=O)c1c(Cl)cnn1[C@H]1CC[C@@H](NC(=O)OC(C)(C)C)CC1] | 534 | 0.58 |
| 310 | 4-chloro-1-[6-(1,4-dioxane-2- carbonyl)-6-azaspiro[3.4]octan-2-yl]- N-{5-[(4-fluorophenyl)ethynyl]-3- methylpyridin-2-yl}-1H-pyrazole-5- carboxamide | 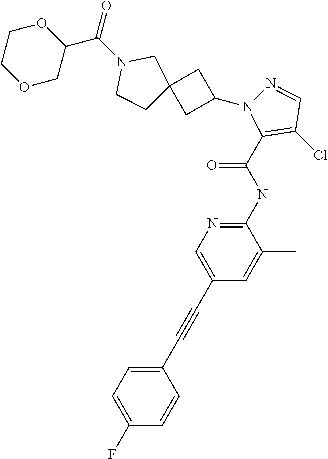 [SMILES: Cc1cc(C#Cc2ccc(F)cc2)cnc1NC(=O)c1c(Cl)cnn1C1CC2(CCN(C(=O)C3COCCO3)C2)C1] | 578.4 | 0.58 |
| 311 | 1-(2-(1-acetylpiperidin-4-yl)ethyl)-4- chloro-N-(3-fluoro-5-(thiophen-2- ylethynyl)pyridin-2-yl)-1H- pyrazole-5-carboxamide | 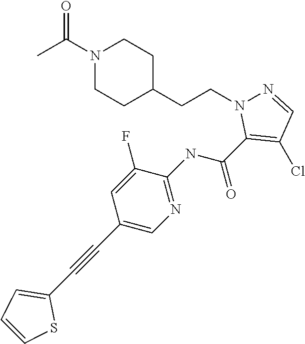 [SMILES: CC(=O)N1CCC(CCn2ncc(Cl)c2C(=O)Nc2ncc(C#Cc3cccs3)cc2F)CC1] | 500.3 | 0.59 |
| 312 | 1-(6-acetyl-6-azaspiro[3.4]octan-2-yl)- 4-chloro-N-{5-[(4- fluorophenyl)ethynyl]-3- methylpyridin-2-yl}-1H-pyrazole-5- carboxamide | 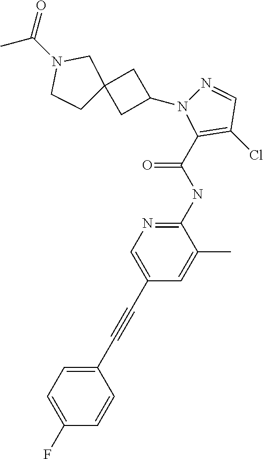 [SMILES: CC(=O)N1CCC2(CC(n3ncc(Cl)c3C(=O)Nc3ncc(C#Cc4ccc(F)cc4)cc3C)C2)C1] | 506.3 | 0.59 |
| 313 | 1-((1-acetyl-3,3-difluoropiperidin-4- yl)methyl)-4-chloro-N-(5-((4- fluorophenyl)ethynyl)-3- methylpyridin-2-yl)-1H- pyrazole-5-carboxamide | 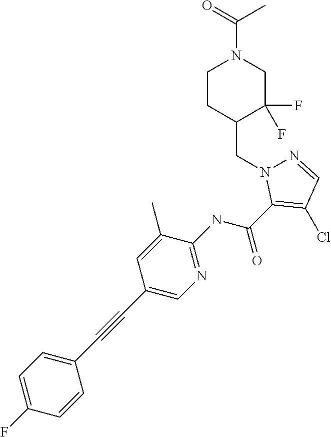 [SMILES: CC(=O)N1CCC(Cn2ncc(Cl)c2C(=O)Nc2ncc(C#Cc3ccc(F)cc3)cc2C)C(F)(F)C1] | 530.2 | 0.59 |
| 314 | 4-chloro-N-(5-((4- fluorophenyl)ethynyl)-3- methylpyridin-2-yl)-1-((3-isobutyryl- 3-azabicyclo[3.1.1]heptan-6- yl)methyl)-1H- pyrazole-5-carboxamide | 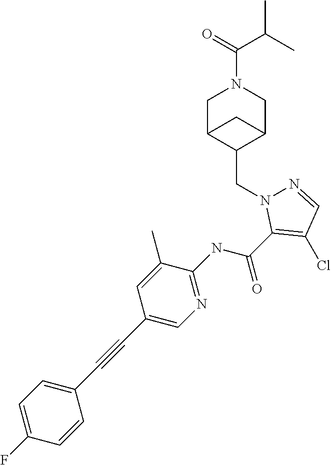 [SMILES: Cc1cc(C#Cc2ccc(F)cc2)cnc1NC(=O)c1c(Cl)cnn1CC1C2CC1CN(C(=O)C(C)C)C2] | 534.4 | 0.59 |
| 315 | tert-butyl (S)-3-(4-chloro-5-((3-fluoro- 5-(phenylethynyl)pyridin-2- yl)carbamoyl)-1H-pyrazol-1- yl)pyrrolidine-1-carboxylate | 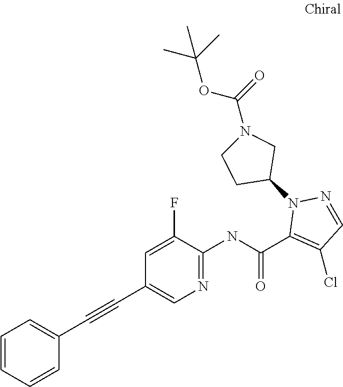 [SMILES: CC(C)(C)OC(=O)N1CC[C@H](n2ncc(Cl)c2C(=O)Nc2ncc(C#Cc3ccccc3)cc2F)C1] | 454 (M − tBu + 2H) | 0.6 |
| 316 | (S)-4-chloro-N-(3-methyl-5-(p- tolylethynyl)pyridin-2-yl)-1- (tetrahydrofuran-3-yl)-1H-pyrazole-5- carboxamide | 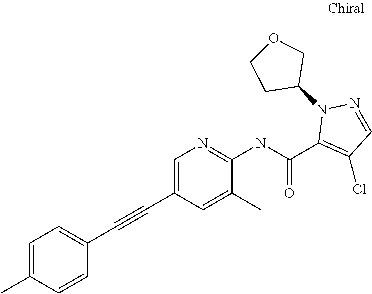 [SMILES: Cc1ccc(C#Cc2cnc(NC(=O)c3c(Cl)cnn3[C@H]3CCOC3)c(C)c2)cc1] | 421.4 | 0.61 |
| 317 | 4-chloro-N-[3-methyl-5- (phenylethynyl)pyridin-2-yl]-1- [(3aR,5s,6aS)-2-(2- methylpropanoyl)octahydrocyclopenta [c]pyrrol-5-yl]-1H- pyrazole-5-carboxamide | 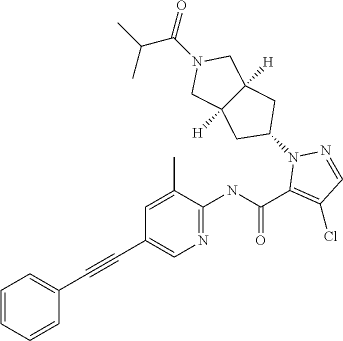 [SMILES: Cc1cc(C#Cc2ccccc2)cnc1NC(=O)c1c(Cl)cnn1[C@H]1C[C@@H]2CN(C(=O)C(C)C)C[C@@H]2C1] | 516 | 0.61 |
| 318 | tert-butyl 4-((4-chloro-5-((3-fluoro-5- (phenylethynyl)pyridin-2- yl)carbamoyl)-1H-pyrazol-1- yl)methyl)-3-methylpiperidine-1- carboxylate | 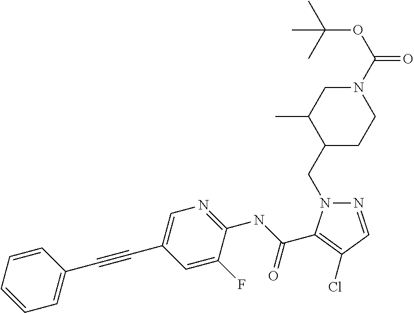 [SMILES: CC1CN(C(=O)OC(C)(C)C)CCC1Cn1ncc(Cl)c1C(=O)Nc1ncc(C#Cc2ccccc2)cc1F] | 552.4 | 0.61 |
| 319 | 4-chloro-N-[3-fluoro-5- (phenylethynyl)pyridin-2-yl]-1- {[(1S,2S,4R)-7- oxabicyclo[2.2.1]heptan-2-yl]methyl}- 1H-pyrazole-5-carboxamide and 4-chloro-N-[3-fluoro-5- {[(1R,2R,4S)-7- oxabicyclo[2.2.1]heptan-2-yl)methyl}- 1H-pyrazole-5-carboxamide | 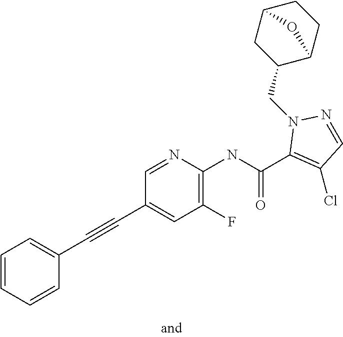 [SMILES: O=C(Nc1ncc(C#Cc2ccccc2)cc1F)c1c(Cl)cnn1C[C@@H]1C[C@H]2CC[C@@H]1O2] | 451 | 0.62 |
| | | 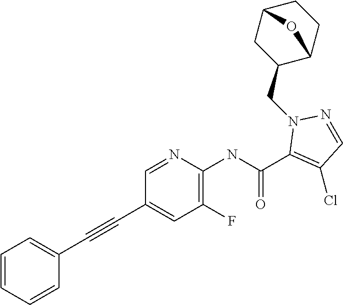 [SMILES: O=C(Nc1ncc(C#Cc2ccccc2)cc1F)c1c(Cl)cnn1C[C@H]1C[C@@H]2CC[C@H]1O2] | | |
| 320 | (S)-4-chloro-N-(5-((4- fluorophenyl)ethynyl)-3- methylpyridin-2-yl)-1-((1- isobutyrylpiperidin-3-yl)methyl)-1H- pyrazole-5-carboxamide | 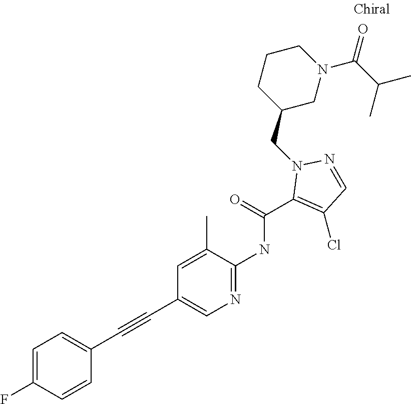 [SMILES: Cc1cc(C#Cc2ccc(F)cc2)cnc1NC(=O)c1c(Cl)cnn1C[C@H]1CCCN(C(=O)C(C)C)C1] | 522.3 | 0.62 |
| 321 | 4-chloro-N-(3-methyl-5- (phenylethynyl)pyridin-2-yl)-1-(2- (tetrahydrofuran-2-yl)ethyl)-1H- pyrazole-5-carboxamide | 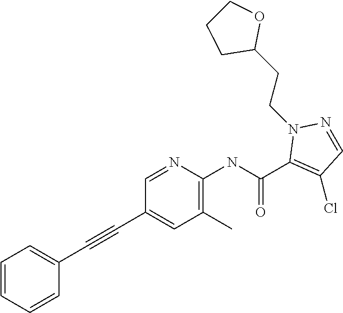 [SMILES: Cc1cc(C#Cc2ccccc2)cnc1NC(=O)c1c(Cl)cnn1CCC1CCCO1] | 435 | 0.63 |
| 322 | (S)-4-chloro-N-(5-((4- fluorophenyl)ethynyl)-3- methylpyridin-2-yl)-1- (tetrahydrofuran-3-yl)-1H- pyrazole-5-carboxamide | 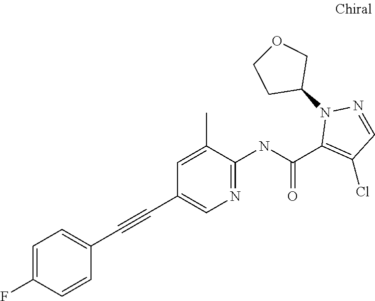 [SMILES: Cc1cc(C#Cc2ccc(F)cc2)cnc1NC(=O)c1c(Cl)cnn1[C@H]1CCOC1] | 425 | 0.63 |
| 323 | tert-butyl 4-(2-(4-chloro-5-((3-methyl- 5-(phenylethynyl)pyridin-2- yl)carbamoyl)-1H-pyrazol-1- yl)ethyl)piperazine-1-carboxylate | 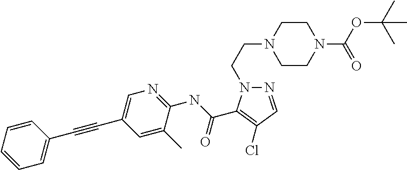 [SMILES: Cc1cc(C#Cc2ccccc2)cnc1NC(=O)c1c(Cl)cnn1CCN1CCN(C(=O)OC(C)(C)C)CC1] | 549.3 | 0.63 |
| 324 | tert-butyl 4-(4-chloro-5-((3-fluoro-5- (phenylethynyl)pyridin-2- yl)carbamoyl)-1H-pyrazol-1-yl)-2- methylpiperidine-1-caboxylate | 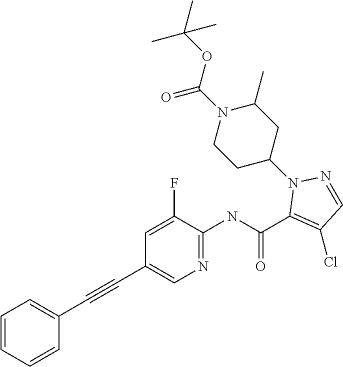 [SMILES: CC1CC(n2ncc(Cl)c2C(=O)Nc2ncc(C#Cc3ccccc3)cc2F)CCN1C(=O)OC(C)(C)C] | 534 | 0.64 |
| 325 | tert-butyl (cis-3-(4-chloro-5-((3- fluoro-5-(phenylethynyl)pyridin-2- yl)carbamoyl)-1H-pyrazol-1- yl)cyclobutyl)carbamate | 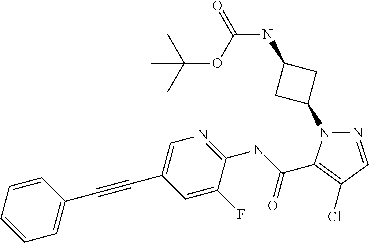 [SMILES: CC(C)(C)OC(=O)N[C@H]1C[C@@H](n2ncc(Cl)c2C(=O)Nc2ncc(C#Cc3ccccc3)cc2F)C1] | 510.3 | 0.64 |
| 326 | 1-((3-acetyl-3- azabicyclo[3.1.1]heptan-6-yl)methyl)- 4-chloro-N-(3-fluoro-5- (phenylethynyl)pyridin-2-yl)-1H- pyrazole-5-carboxamide | 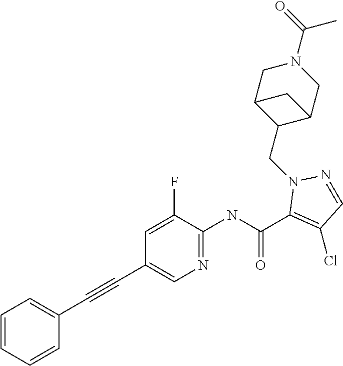 [SMILES: CC(=O)N1CC2CC(C1)C2Cn1ncc(Cl)c1C(=O)Nc1ncc(C#Cc2ccccc2)cc1F] | 492.2 | 0.64 |
| 327 | 4-chloro-N-(3-methyl-5- (phenylethynyl)pyridin-2-yl)-1- (tetrahydro-2H-pyran-3-yl)-1H- pyrazole-5-carboxamide | 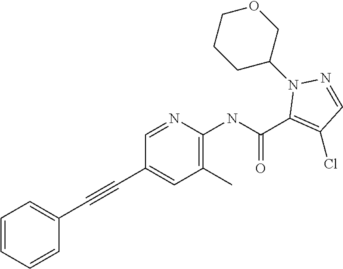 [SMILES: Cc1cc(C#Cc2ccccc2)cnc1NC(=O)c1c(Cl)cnn1C1CCCOC1] | 421 | 0.65 |
| 328 | tert-butyl 4-(4-chloro-5-((3-methyl-5- (phenylethynyl)pyridin-2- yl)carbamoyl)-1H-pyrazol-1- yl)piperidine-1-carboxylate | 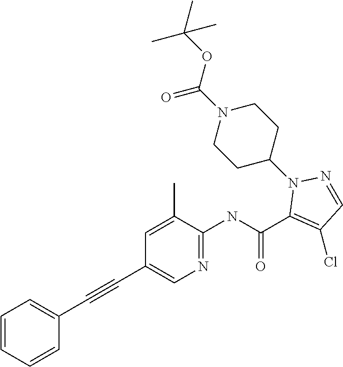 [SMILES: Cc1cc(C#Cc2ccccc2)cnc1NC(=O)c1c(Cl)cnn1C1CCN(C(=O)OC(C)(C)C)CC1] | 520 | 0.65 |
| 329 | tert-butyl (3aR,5s,6aS)-5-(4-chloro-5- {[3-methyl-5-(phenylethynyl)pyridin- 2-yl]carbamoyl}-1H-pyrazol-1- yl)hexahydrocyclopenta[c]pyrrole- 2(1H)-carboxylate | 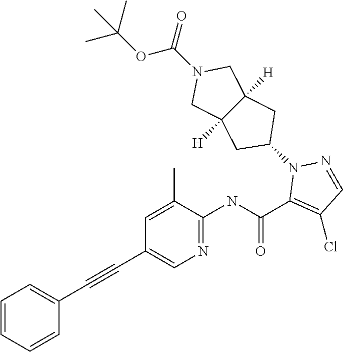 [SMILES: Cc1cc(C#Cc2ccccc2)cnc1NC(=O)c1c(Cl)cnn1[C@H]1C[C@@H]2CN(C(=O)OC(C)(C)C)C[C@@H]2C1] | 546.4 | 0.65 |
| 330 | tert-butyl 6-(4-chloro-5-((3-methyl-5- (phenylethynyl)pyridin-2- yl)carbamoyl)-1H-pyrazol-1-yl)-2- azaspiro[3.3]heptane-2-carboxylate | 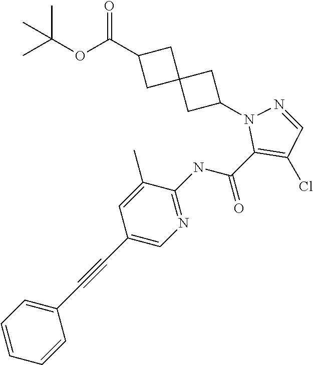 [SMILES: Cc1cc(C#Cc2ccccc2)cnc1NC(=O)c1c(Cl)cnn1C1CC2(CC(C(=O)OC(C)(C)C)C2)C1] | 532.4 | 0.65 |
| 331 | 1-[(3aR,5s,6aS)-2- acetyloctahydrocyclopenta[c]pyrrol-5- yl]-4-chloro-N-{5-[(4- fluorophenyl)ethynyl]-3- methylpyridin-2-yl}-1H-pyrazole-5- carboxamide | 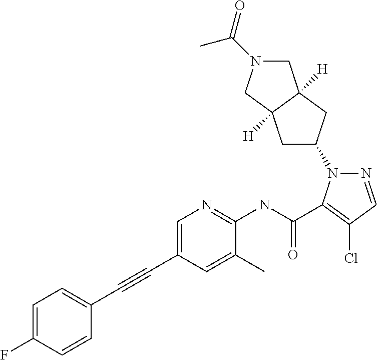 [SMILES: CC(=O)N1C[C@H]2C[C@H](n3ncc(Cl)c3C(=O)Nc3ncc(C#Cc4ccc(F)cc4)cc3C)C[C@H]2C1] | 506.4 | 0.66 |
| 332 | 4-chloro-N-{5-[(4- fluorophenyl)ethynyl]-3- methylpyridin-2-yl}-1-[6-(2- methylpropanoyl)-6- azaspiro[3.4]octan-2-yl]-1H-pyrazole- 5-carboxamide | 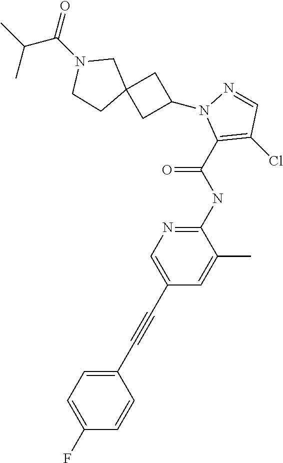 [SMILES: Cc1cc(C#Cc2ccc(F)cc2)cnc1NC(=O)c1c(Cl)cnn1C1CC2(CCN(C(=O)C(C)C)C2)C1] | 534.2 | 0.66 |
| 333 | (S)-4-chloro-N-(5-((3- fluorophenyl)ethynyl)-3- methylpyridin-2-yl)-1- (tetrahydrofuran-3-yl)-1H- pyrazole-5-carboxamide | 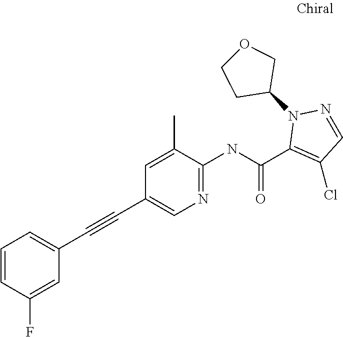 [SMILES: Cc1cc(C#Cc2cccc(F)c2)cnc1NC(=O)c1c(Cl)cnn1[C@H]1CCOC1] | 425 | 0.67 |
| 334 | tert-butyl 6-(4-chloro-5-((3-fluoro-5- (phenylethynyl)pyridin-2- yl)carbamoyl)-1H-pyrazol-1-yl)-2- azaspiro[3.3]heptane-2-carboxylate | 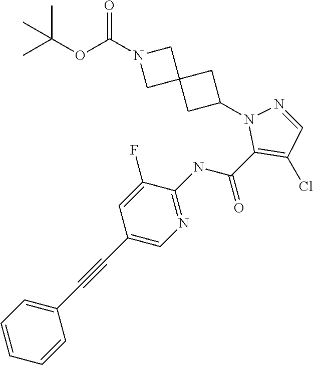 [SMILES: CC(C)(C)OC(=O)N1CC2(CC(n3ncc(Cl)c3C(=O)Nc3ncc(C#Cc4ccccc4)cc3F)C2)C1] | 536.3 | 0.67 |
| 335 | tert-butyl 4-(4-chloro-5-((3-fluoro-5- (thiophen-2-ylethynyl)pyridin-2- yl)carbamoyl)-1H-pyrazol-1- yl)piperidine-1-carboxylate | 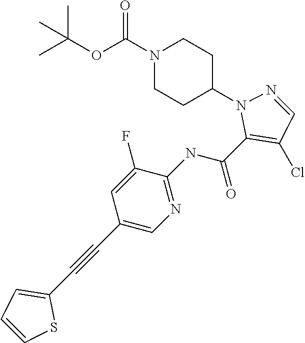 [SMILES: CC(C)(C)OC(=O)N1CCC(n2ncc(Cl)c2C(=O)Nc2ncc(C#Cc3cccs3)cc2F)CC1] | 474.3 (M − tBu + 2H) | 0.7 |
| 336 | tert-butyl (3R,4R)-4-(4-chloro-5-((3- methyl-5-(phenylethynyl)pyridin-2- yl)carbamoyl)-1H-pyrazol-1-yl)-3- fluoropiperidine-1-carboxylate | 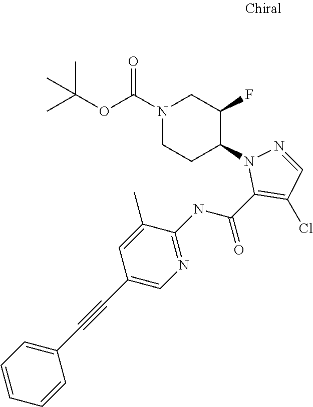 [SMILES: Cc1cc(C#Cc2ccccc2)cnc1NC(=O)c1c(Cl)cnn1[C@H]1CCN(C(=O)OC(C)(C)C)C[C@H]1F] | 538 | 0.71 |
| 337 | tert-butyl (3R,4R)-4-(4-chloro-5-((3- fluoro-5-(phenylethynyl)pyridin-2- yl)carbamoyl)-1H-pyrazol-1-yl)-3- fluoropiperidine-1-carboxylate | 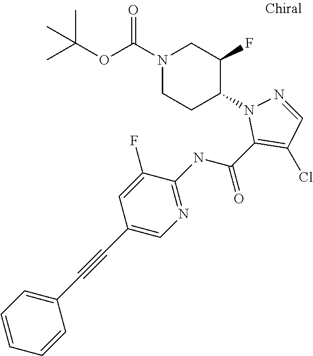 [SMILES: CC(C)(C)OC(=O)N1CC[C@@H](n2ncc(Cl)c2C(=O)Nc2ncc(C#Cc3ccccc3)cc2F)[C@H](F)C1] | 485.9 (M − tBu + 2H) | 0.72 |
| 338 | (R)-1-((1,4-dioxan-2-yl)methyl)-4- chloro-N-(3-methyl-5- (phenylethynyl)pyridin-2-yl)-1H- pyrazole-5-carboxamide | 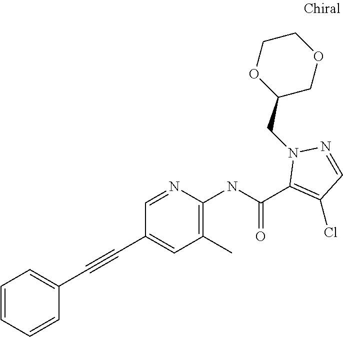 [SMILES: Cc1cc(C#Cc2ccccc2)cnc1NC(=O)c1c(Cl)cnn1C[C@@H]1COCCO1] | 437 | 0.73 |
| 339 | 4-chloro-1-((4-fluoro-1- isobutyrylpiperidin-4-yl)methyl)-N-(5- ((4-fluorophenyl)ethynyl)-3- methylpyridin-2-yl)-1H- pyrazole-5-carboxamide | 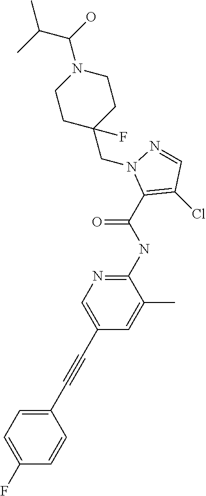 [SMILES: Cc1cc(C#Cc2ccc(F)cc2)cnc1NC(=O)c1c(Cl)cnn1CC1(F)CCN(C(O)C(C)C)CC1] | 540.2 | 0.73 |
| 340 | 4-chloro-1-((2,6-dimethyltetrahydro- 2H-pyran-4-yl)methyl)-N-(3-methyl- 5-(phenylethynyl)pyridin-2-yl)-1H- pyrazole-5-carboxamide | 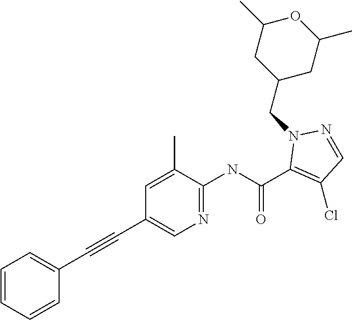 | 463 | 0.74 |
| 341 | 1-(((3S,4S)-1-acetyl-3-fluoropiperidin- 4-yl)methyl)-4-chloro-N-(5-((4- fluorophenyl)ethynyl)-3- methylpyridin-2-yl)-1H- pyrazole-5-carboxamide | 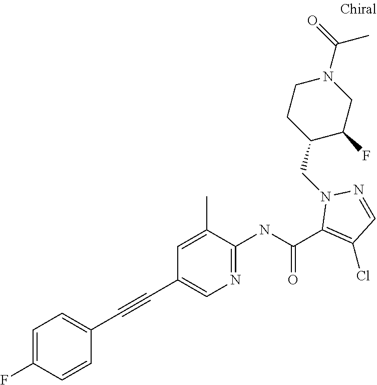 [SMILES: CC(=O)N1CC[C@@H](Cn2ncc(Cl)c2C(=O)Nc2ncc(C#Cc3ccc(F)cc3)cc2C)[C@H](F)C1] | 512.2 | 0.74 |
| 342 | 3-chloro-N-(5-((4- fluorophenyl)ethynyl)-3- methylpyridin-2-yl)-1-methyl-5-(1- methyl-1H-indazol-5-yl)-1H- pyrazole-4-carboxamide | 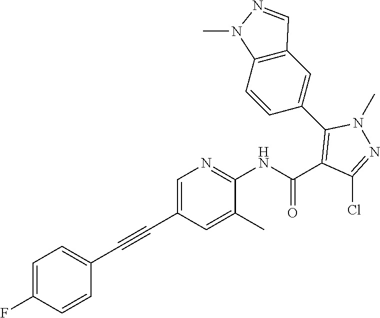 [SMILES: Cc1cc(C#Cc2ccc(F)cc2)cnc1NC(=O)c1c(Cl)nn(C)c1-c1ccc2c(cnn2C)c1] | 499.2 | 0.75 |
| 343 | (S)-1-((1,4-dioxan-2-yl)methyl)-4- chloro-N-(3-methyl-5-(thiophen-2- ylethynyl)pyridin-2-yl)-1H- pyrazole-5-carboxamide | 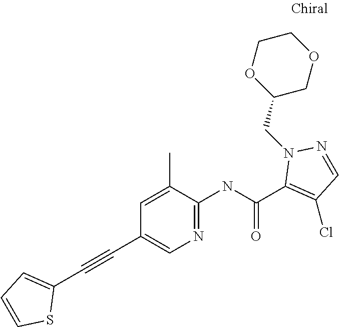 [SMILES: Cc1cc(C#Cc2cccs2)cnc1NC(=O)c1c(Cl)cnn1C[C@H]1COCCO1] | 443 | 0.76 |
| 344 | tert-butyl (R)-3-((4-chloro-5-((5-((4- fluorophenyl)ethynyl)-3- methylpyridin-2-yl)carbamoyl)-1H- pyrazol-1-yl)methyl)piperidine-1- carboxylate | 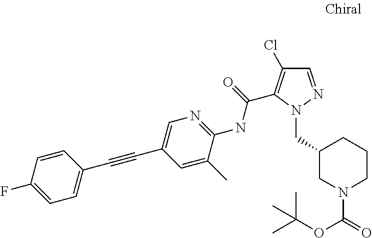 [SMILES: Cc1cc(C#Cc2ccc(F)cc2)cnc1NC(=O)c1c(Cl)cnn1C[C@@H]1CCCN(C(=O)OC(C)(C)C)C1] | 552.3 | 0.76 |
| 345 | 4-chloro-N-(3-methyl-5- (phenylethynyl)pyridin-2-yl)-1- ((tetrahydrofuran-2-yl)methyl)-1H- pyrazole-5-carboxamide | 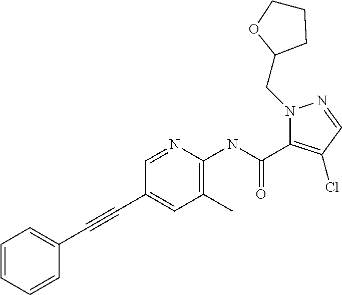 [SMILES: Cc1cc(C#Cc2ccccc2)cnc1NC(=O)c1c(Cl)cnn1CC1CCCO1] | 421 | 0.77 |
| 346 | tert-butyl 2-(4-chloro-5-((3-fluoro-5- (phenylethynyl)pyridin-2- yl)carbamoyl)-1H-pyrazol-1-yl)-7- azaspiro[3.5]nonane-7-carboxylate | 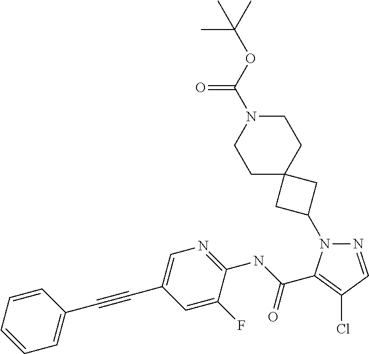 [SMILES: CC(C)(C)OC(=O)N1CCC2(CC1)CC(n1ncc(Cl)c1C(=O)Nc1ncc(C#Cc3ccccc3)cc1F)C2] | 564.3 | 0.77 |
| 347 | 4-chloro-N-(5-((4- fluorophenyl)ethynyl)-3- methylpyridin-2-yl)-1-((4- propionylmorpholin-2-yl)methyl)-1H- pyrazole-5-carboxamide | 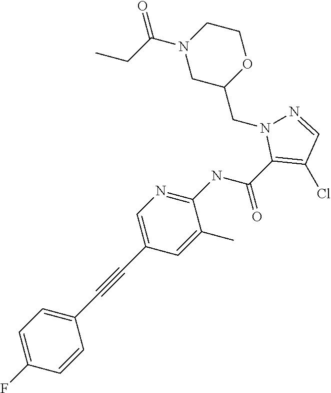 [SMILES: CCC(=O)N1CCOC(Cn2ncc(Cl)c2C(=O)Nc2ncc(C#Cc3ccc(F)cc3)cc2C)C1] | 510.2 | 0.77 |
| 348 | 4-chloro-1-((4-fluoro-1-(2- methylcyclopropane-1- carbonyl)piperidin-4-yl)methyl)-N-(5- ((4-fluorophenyl)ethynyl)-3- methylpyridin-2-yl)-1H- pyrazole-5-carboxamide | 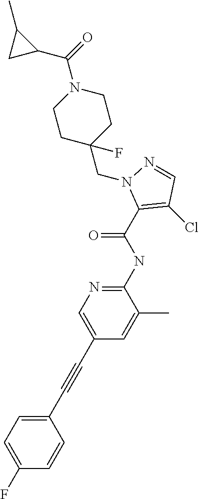 [SMILES: Cc1cc(C#Cc2ccc(F)cc2)cnc1NC(=O)c1c(Cl)cnn1CC1(F)CCN(C(=O)C2CC2C)CC1] | 552.2 | 0.78 |
| 349 | 4-chloro-N-{5-[(3- fluorophenyl)ethynyl]-3- methylpyridin-2-yl}-1-[(3aR,5s,6aS)- 2-(2- methylpropanoyl)octahydrocyclopenta [c]pyrrol-5-yl]-1H- pyrazole-5-carboxamide | 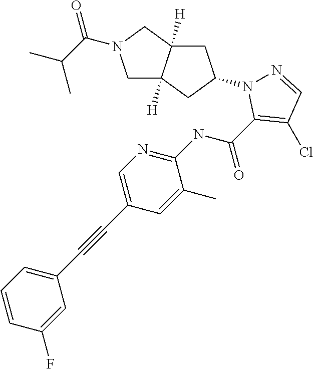 [SMILES: Cc1cc(C#Cc2cccc(F)c2)cnc1NC(=O)c1c(Cl)cnn1[C@H]1C[C@@H]2CN(C(=O)C(C)C)C[C@@H]2C1] | 534.3 | 0.81 |
| 350 | 4-chloro-1-((3,3-difluoro-1- isobutyrylpiperidin-4-yl)methyl)-N-(5- ((4-fluorophenyl)ethynyl)-3- methylpyridin-2-yl)-1H- pyrazole-5-carboxamide | 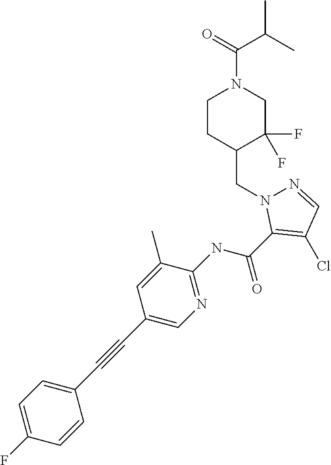 [SMILES: Cc1cc(C#Cc2ccc(F)cc2)cnc1NC(=O)c1c(Cl)cnn1CC1CCN(C(=O)C(C)C)CC1(F)F] | 558.2 | 0.82 |
| 351 | 1-(trans-3-acetamidocyclobutyl)-4- chloro-N-(5-((4- fluorophenyl)ethynyl)-3- methylpyridin-2-yl)-1H- pyrazole-5-carboxamide | 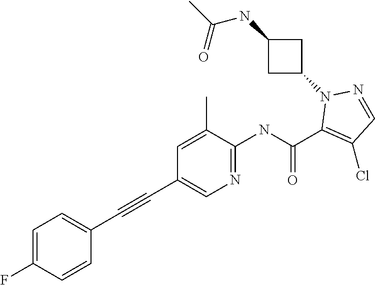 [SMILES: CC(=O)N[C@H]1C[C@H](n2ncc(Cl)c2C(=O)Nc2ncc(C#Cc3ccc(F)cc3)cc2C)C1] | 466.2 | 0.82 |
| 352 | 4-chloro-1-[(3aR,5s,6aS)-2-(2,2- dimethylpropanoyl)octahydrocyclopenta [c]pyrrol-5-yl]-N-[3-methyl-5- (phenylethynyl)pyridin-2-yl]-1H- pyrazole-5-carboxamide | 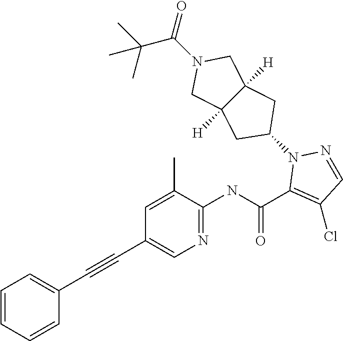 [SMILES: Cc1cc(C#Cc2ccccc2)cnc1NC(=O)c1c(Cl)cnn1[C@H]1C[C@@H]2CN(C(=O)C(C)(C)C)C[C@@H]2C1] | 530.4 | 0.84 |
| 353 | tert-butyl (R)-3-(4-chloro-5-((3- methyl-5-(phenylethynyl)pyridin-2- yl)carbamoyl)-1H-pyrazol-1- yl)pyrrolidine-1-carboxylate | 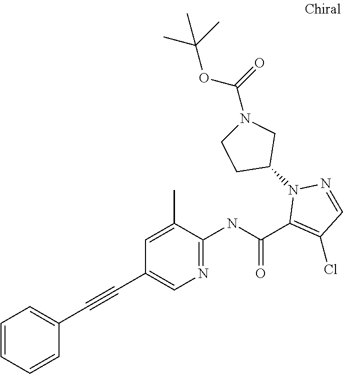 [SMILES: Cc1cc(C#Cc2ccccc2)cnc1NC(=O)c1c(Cl)cnn1[C@@H]1CCN(C(=O)OC(C)(C)C)C1] | 506 | 0.85 |
| 354 | 4-chloro-1-[(3aR,5s,6aS)-2-(2,2- dimethylpropanoyl)octahydrocyclopenta [c]pyrrol-5-yl]-N-{3-methyl-5- [(thiophen-2-yl)ethynyl]pyridin-2-yl}- 1H-pyrazole-5-carboxamide | 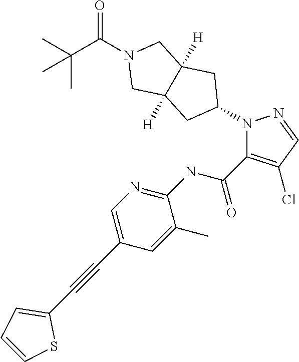 [SMILES: Cc1cc(C#Cc2cccs2)cnc1NC(=O)c1c(Cl)cnn1[C@H]1C[C@@H]2CN(C(=O)C(C)(C)C)C[C@@H]2C1] | 536.4 | 0.85 |
| 355 | 4-chloro-N-(5-((4- fluorophenyl)ethynyl)-3- methylpyridin-2-yl)-1-((1- propionylpiperidin-3-yl)methyl)-1H- pyrazole-5-carboxamide | 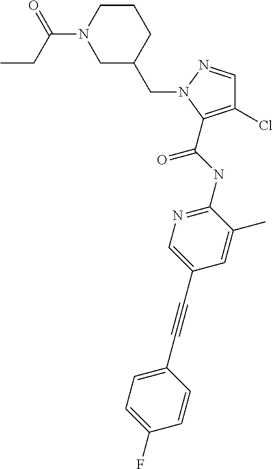 [SMILES: CCC(=O)N1CCCC(Cn2ncc(Cl)c2C(=O)Nc2ncc(C#Cc3ccc(F)cc3)cc2C)C1] | 508.3 | 0.85 |
| 356 | 1-((1-acetylpiperidin-4-yl)methyl)-4- chloro-N-(5-((2- fluorophenyl)ethynyl)-3- methylpyridin-2-yl)-1H- pyrazole-5-carboxamide | 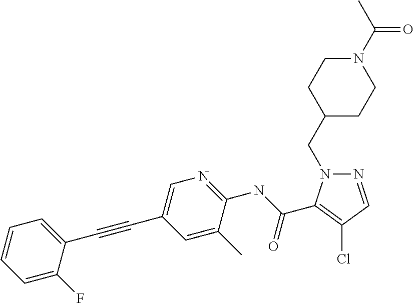 [SMILES: CC(=O)N1CCC(Cn2ncc(Cl)c2C(=O)Nc2ncc(C#Cc3ccccc3F)cc2C)CC1] | 494.4 | 0.86 |
| 357 | tert-butyl (cis-3-(4-chloro-5-((3- methyl-5-(phenylethynyl)pyridin-2- yl)carbamoyl)-1H-pyrazol-1- yl)cyclobutyl)carbamate | 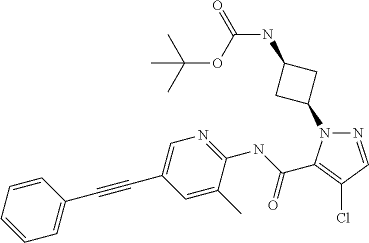 [SMILES: Cc1cc(C#Cc2ccccc2)cnc1NC(=O)c1c(Cl)cnn1[C@H]1C[C@@H](NC(=O)OC(C)(C)C)C1] | 506.4 | 0.87 |
| 358 | tert-butyl (3-(4-chloro-5-((3-methyl-5- (phenylethynyl)pyridin-2- yl)carbamoyl)-1H-pyrazol-1- yl)cyclohexyl)carbamate | 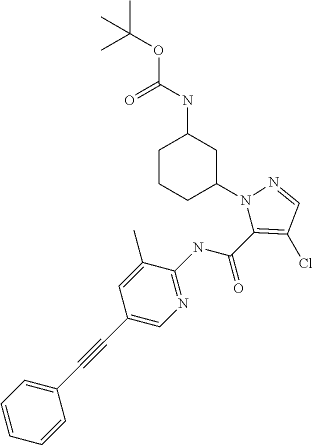 [SMILES: Cc1cc(C#Cc2ccccc2)cnc1NC(=O)c1c(Cl)cnn1C1CCCC(NC(=O)OC(C)(C)C)C1] | 534.2 | 0.88 |
| 359 | 1-[(3aR,5s,6aS)-2- acetyloctahydrocyclopenta[c]pyrrol-5- yl]-4-chloro-N-{5-[(2- fluorophenyl)ethynyl]-3- methylpyridin-2-yl}-1H- pyrazole-5-carboxamide | 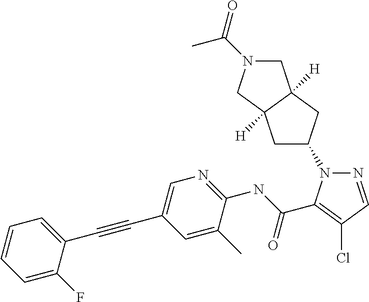 [SMILES: CC(=O)N1C[C@H]2C[C@H](n3ncc(Cl)c3C(=O)Nc3ncc(C#Cc4ccccc4F)cc3C)C[C@H]2C1] | 506.3 | 0.9 |
| 360 | tert-butyl (trans-4-(4-chloro-5-((3- fluoro-5-(phenylethynyl)pyridin-2- yl)carbamoyl)-1H-pyrazol-1- yl)cyclohexyl)carbamate | 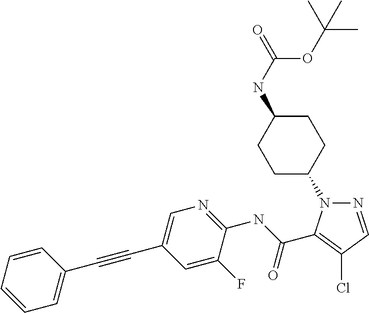 [SMILES: CC(C)(C)OC(=O)N[C@H]1CC[C@H](n2ncc(Cl)c2C(=O)Nc2ncc(C#Cc3ccccc3)cc2F)CC1] | 538.3 | 0.9 |
| 361 | (S)-1-((1,4-dioxan-2-yl)methyl)-4- chloro-N-(3-chloro-5- (phenylethynyl)pyridin-2-yl)-1H- pyrazole-5-carboxamide | 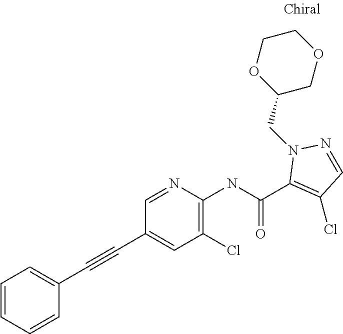 [SMILES: O=C(Nc1ncc(C#Cc2ccccc2)cc1Cl)c1c(Cl)cnn1C[C@H]1COCCO1] | 457 | 0.91 |
| 362*⁸ | 4-chloro-1-[(2s,4r)-6- (cyclopropanecarbonyl)-6- azaspiro[3.4]octan-2-yl]-N-{5-[(4- fluorophenyl)ethynyl]-3- methylpyridin-2-yl}-1H-pyrazole-5- carboxamide or 4-chloro-1-[(2r,4s)-6- (cyclopropanecarbonyl)-6- azaspiro[3.4]octan-2-yl]-N-{5-[(4- fluorophenyl)ethynyl]-3- methylpyridin-2-yl}-1H- pyrazole-5-carboxamide | 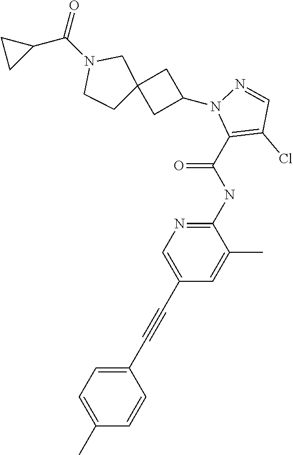 [SMILES: Cc1ccc(C#Cc2cnc(NC(=O)c3c(Cl)cnn3C3CC4(CCN(C(=O)C5CC5)C4)C3)c(C)c2)cc1] | 532.2 | 0.91 |
| 363*⁹ | 4-chloro-1-[(2s,4r)-6- (cyclopropanecarbonyl)-6- azaspiro[3.4]octan-2-yl]-N-{5-[(4- fluorophenyl)ethynyl]-3- methylpyridin-2-yl}-1H- pyrazole-5-carboxamide or 4-chloro-1-[(2r,4s)-6- (cyclopropanecarbonyl)-6- azaspiro[3.4]octan-2-yl]-N-{5-[(4- fluorophenyl)ethynyl]-3- methylpyridin-2-yl}-1H- pyrazole-5-carboxamide | 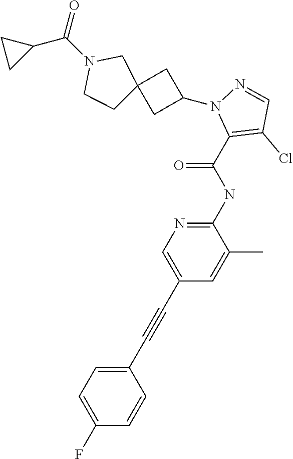 [SMILES: Cc1cc(C#Cc2ccc(F)cc2)cnc1NC(=O)c1c(Cl)cnn1C1CC2(CCN(C(=O)C3CC3)C2)C1] | 532.2 | 0.91 |
| 364 | 3-chloro-1-methyl-5-(1-methyl-1H- indazol-5-yl)-N-(3-methyl-5- (phenylethynyl)pyridin-2-yl)-1H- pyrazole-4-carboxamide | 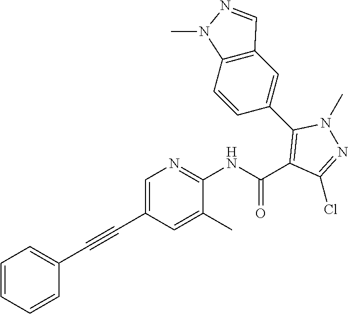 [SMILES: Cc1cc(C#Cc2ccccc2)cnc1NC(=O)c1c(Cl)nn(C)c1-c1ccc2c(cnn2C)c1] | 481.3 | 0.91 |
| 365 | tert-butyl 3-((4-chloro-5-((3-fluoro-5- (phenylethynyl)pyridin-2- yl)carbamoyl)-1H-pyrazol-1- yl)methyl)piperidine-1-carboxylate | 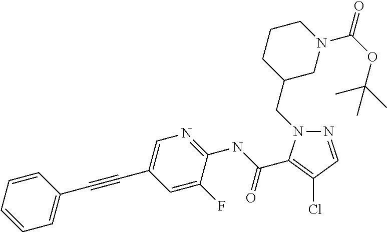 [SMILES: CC(C)(C)OC(=O)N1CCCC(Cn2ncc(Cl)c2C(=O)Nc2ncc(C#Cc3ccccc3)cc2F)C1] | 538.3 | 0.92 |
| 366 | 4-chloro-N-(3-fluoro-5- (phenylethynyl)pyridin-2-yl)-1- ((tetrahydrofuran-2-yl)methyl)-1H- pyrazole-5-carboxamide | 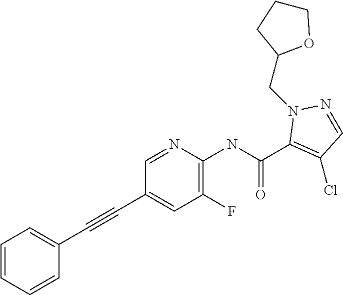 [SMILES: O=C(Nc1ncc(C#Cc2ccccc2)cc1F)c1c(Cl)cnn1CC1CCCO1] | 425 | 0.93 |
| 367 | 4-chloro-1-((4- (cyclobutanecarbonyl)morpholin-2- yl)methyl)-N-(5-((4- fluorophenyl)ethynyl)-3- methylpyridin-2-yl)-1H- pyrazole-5-carboxamide | 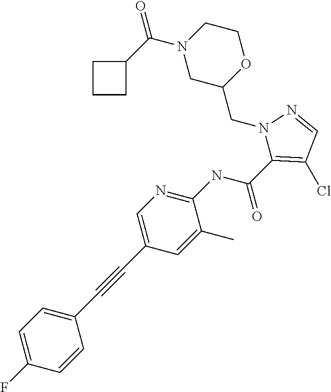 [SMILES: Cc1cc(C#Cc2ccc(F)cc2)cnc1NC(=O)c1c(Cl)cnn1CC1CN(C(=O)C2CCC2)CCO1] | 536.3 | 0.94 |
| 368 | tert-butyl (S)-3-((4-chloro-5-((5-((4- fluorophenyl)ethynyl)-3- methylpyridin-2-yl)carbamoyl)-1H- pyrazol-1-yl)methyl)piperidine-1- carboxylate | 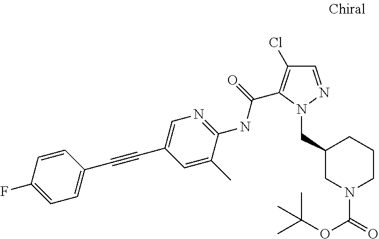 [SMILES: Cc1cc(C#Cc2ccc(F)cc2)cnc1NC(=O)c1c(Cl)cnn1C[C@H]1CCCN(C(=O)OC(C)(C)C)C1] | 552.3 | 0.94 |
| 369 | 4-chloro-1-(2-methoxyethyl)-N-(3- methyl-5-(phenylethynyl)pyridin-2- yl)-1H-pyrazole-5-carboxamide | 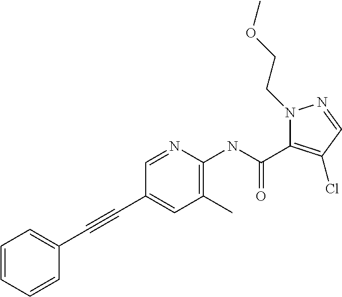 [SMILES: COCCn1ncc(Cl)c1C(=O)Nc1ncc(C#Cc2ccccc2)cc1C] | 395 | 0.95 |
| 370 | 1-((3-acetyl-3- azabicyclo[3.1.1]heptan-6-yl)methyl)- 4-chloro-N-(3-methyl-5- (phenylethynyl)pyridin-2-yl)-1H- pyrazole-5-carboxamide | 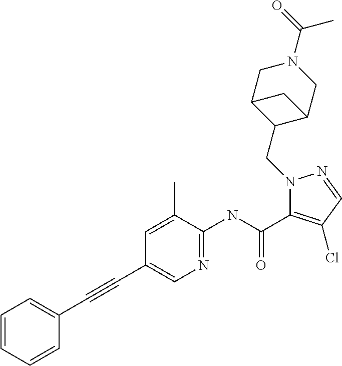 [SMILES: CC(=O)N1CC2CC(C1)C2Cn1ncc(Cl)c1C(=O)Nc1ncc(C#Cc2ccccc2)cc1C] | 488.2 | 0.98 |
| 371 | 4-chloro-1-((5,5- dimethyltetrahydrofuran-2-yl)methyl)- N-(3-methyl-5- (phenylethynyl)pyridin-2-yl)-1H- pyrazole-5-carboxamide | 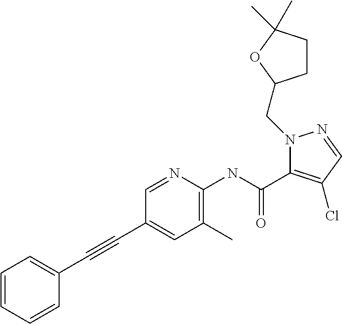 [SMILES: Cc1cc(C#Cc2ccccc2)cnc1NC(=O)c1c(Cl)cnn1CC1CCC(C)(C)O1] | 449 | 1 |
| 372 | 3-chloro-N-(3-fluoro-5- (phenylethynyl)pyridin-2-yl)-1- methyl-5-(1-methyl-1H-indazol-5-yl)- 1H-pyrazole-4-carboxamide | 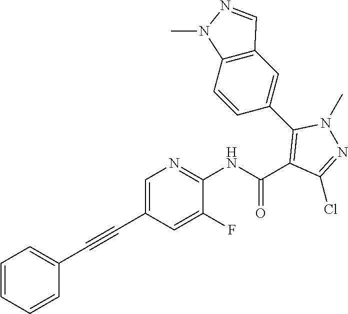 [SMILES: Cn1nc(Cl)c(C(=O)Nc2ncc(C#Cc3ccccc3)cc2F)c1-c1ccc2c(cnn2C)c1] | 485.3 | 1.08 |
| 373 | 4-chloro-1-(1-ethylazetidin-3-yl)-N- (3-fluoro-5-(phenylethynyl)pyridin-2- yl)-1H-pyrazole-5-carboxamide hydrate | 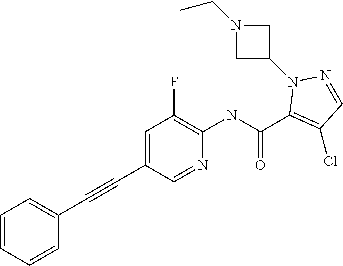 [SMILES: CCN1CC(n2ncc(Cl)c2C(=O)Nc2ncc(C#Cc3ccccc3)cc2F)C1] | 424 | 1.1 |
| 374 | 4-chloro-N-(3-methyl-5- (phenylethynyl)pyridin-2-yl)-1-((2- methylthiazol-5-yl)methyl)-1H- pyrazole-5-carboxamide | 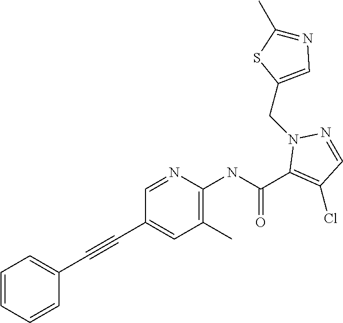 [SMILES: Cc1ncc(Cn2ncc(Cl)c2C(=O)Nc2ncc(C#Cc3ccccc3)cc2C)s1] | 448 | 1.13 |
| 375 | 3-chloro-N-(3-methyl-5- (phenylethynyl)pyridin-2-yl)-1- ((tetrahydro-2H-pyran-4-yl)methyl)- 1H-pyrrole-2-carboxamide | 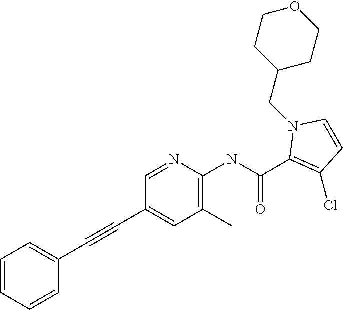 [SMILES: Cc1cc(C#Cc2ccccc2)cnc1NC(=O)c1c(Cl)ccn1CC1CCOCC1] | 434 | 1.15 |
| 376 | (R)-4-chloro-N-(3-chloro-5- (phenylethynyl)pyridin-2-yl)-1- (tetrahydrofuran-3-yl)-1H- pyrazole-5-carboxamide | 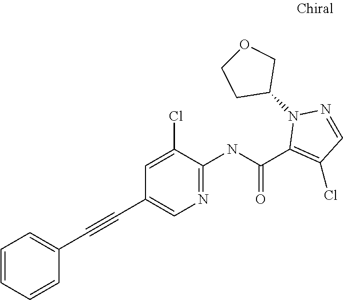 [SMILES: O=C(Nc1ncc(C#Cc2ccccc2)cc1Cl)c1c(Cl)cnn1[C@@H]1CCOC1] | 427 | 1.54 |
| 377 | 4-chloro-1-(1-ethylazetidin-3-yl)-N- (3-methyl-5-(phenylethynyl)pyridin-2- yl)-1H-pyrazole-5-carboxamide | 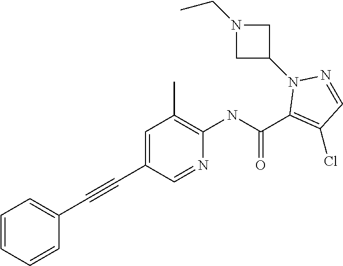 [SMILES: CCN1CC(n2ncc(Cl)c2C(=O)Nc2ncc(C#Cc3ccccc3)cc2C)C1] | 420 | 1.71 |
| 378 | 1-(1-acetylpiperidin-4-yl)-4-chloro-N- (5-fluoro-6-(phenylethynyl)pyridin-3- yl)-1H-pyrazole-5-carboxamide | 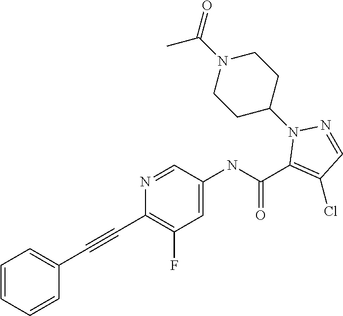 [SMILES: CC(=O)N1CCC(n2ncc(Cl)c2C(=O)Nc2cnc(C#Cc3ccccc3)c(F)c2)CC1] | 466 | 5.68 |
| 379 | (S)-4-chloro-N-(5-(1-(4-fluorophenyl)- 1H-pyrazol-4-yl)-3-methylpyridin-2- yl)-1-((tetrahydrofuran-3-yl)methyl)- 1H-pyrazole-5-carboxamide | 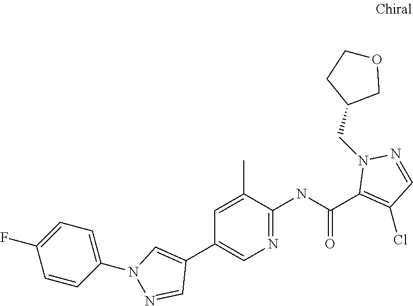 [SMILES: Cc1cc(-c2cnn(-c3ccc(F)cc3)c2)cnc1NC(=O)c1c(Cl)cnn1C[C@@H]1CCOC1] | 481 | 0.32 |
| 380 | 1-(cis-4-(1,4-dioxane-2- carboxamido)cyclohexyl)-N-(4- (benzyloxy)-2-methylphenyl)-4- chloro-1H-pyrazole-5-carboxamide | 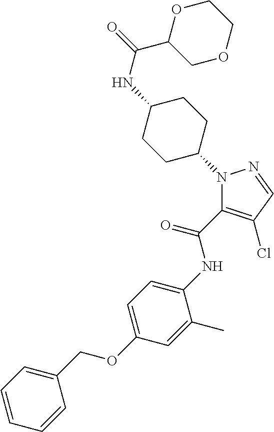 [SMILES: Cc1cc(OCc2ccccc2)ccc1NC(=O)c1c(Cl)cnn1[C@H]1CC[C@@H](NC(=O)C2COCCO2)CC1] | 553.4 | 0.38 |
| 381 | 4-chloro-N-(5-(1-(4-fluorophenyl)-1H- pyrazol-4-yl)-3-methylpyridin-2-yl)-1- (tetrahydro-2H-pyran-4-yl)-1H- pyrazole-5-carboxamide | 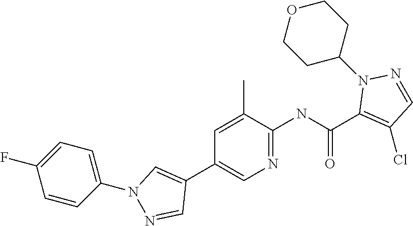 [SMILES: Cc1cc(-c2cnn(-c3ccc(F)cc3)c2)cnc1NC(=O)c1c(Cl)cnn1C1CCOCC1] | 481 | 0.60 |
| 382 | N-(4-(benzyloxy)-2-methylphenyl)-4- chloro-1-(cis-4-(3- methylbutanamido)cyclohexyl)-1H- pyrazole-5-carboxamide | 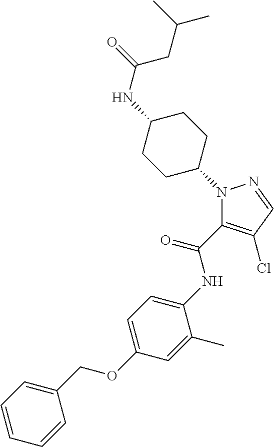 [SMILES: Cc1cc(OCc2ccccc2)ccc1NC(=O)c1c(Cl)cnn1[C@H]1CC[C@@H](NC(=O)CC(C)C)CC1] | 523.2 | 0.61 |
| 383 | tert-butyl 4-(4-chloro-5-((5′-fluoro-6′- methoxy-5-methyl-[3,3′-bipyridin]-6- yl)carbamoyl)-1H-pyrazol-1- yl)piperidine-1-carboxylate | 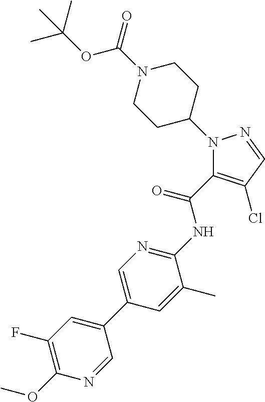 [SMILES: COc1ncc(-c2cnc(NC(=O)c3c(Cl)cnn3C3CCN(C(=O)OC(C)(C)C)CC3)c(C)c2)cc1F] | 545.4 | 0.68 |
| 384 | tert-butyl 4-(4-chloro-5-((5-(3,4- dimethoxyphenyl)-3-methylpyridin-2- yl)carbamoyl)-1H-pyrazol-1- yl)piperidine-1-carboxylate | 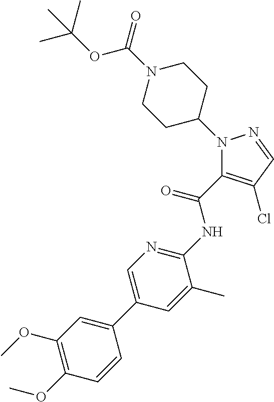 [SMILES: COc1ccc(-c2cnc(NC(=O)c3c(Cl)cnn3C3CCN(C(=O)OC(C)(C)C)CC3)c(C)c2)cc1OC] | 556.4 | 0.74 |
| 385 | N-(4-(benzyloxy)-2-methylphenyl)-4- chloro-1-(cis-4- (cyclopropanecarboxamido)cyclohexyl)- 1H-pyrazole-5-carboxamide | 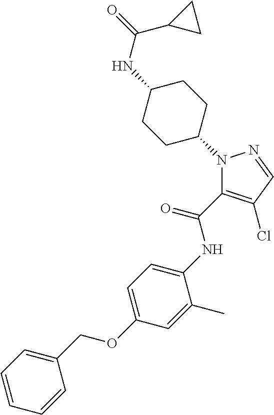 [SMILES: Cc1cc(OCc2ccccc2)ccc1NC(=O)c1c(Cl)cnn1[C@H]1CC[C@@H](NC(=O)C2CC2)CC1] | 507.3 | 0.83 |
| 386 | tert-butyl 4-(4-chloro-5-((5-(3,5- difluoro-4-methoxyphenyl)-3- methylpyridin-2-yl)carbamoyl)-1H- pyrazol-1-yl)piperidine-1-carboxylate | 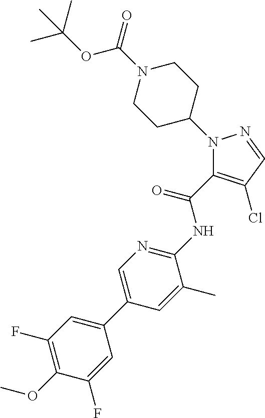 [SMILES: COc1c(F)cc(-c2cnc(NC(=O)c3c(Cl)cnn3C3CCN(C(=O)OC(C)(C)C)CC3)c(C)c2)cc1F] | 562.4 | 0.89 |
| 387 | (R)-4-chloro-N-(5-(1-(4- fluorophenyl)-1H-pyrazol-4-yl)-3- methylpyridin-2-yl)-1- ((tetrahydrofuran-3-yl)methyl)-1H- pyrazole-5-carboxamide | 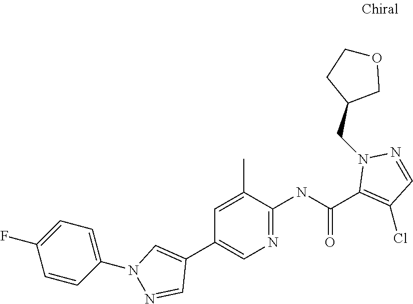 [SMILES: Cc1cc(-c2cnn(-c3ccc(F)cc3)c2)cnc1NC(=O)c1c(Cl)cnn1C[C@H]1CCOC1] | 481 | 0.90 |
| 388 | 1-(1-acetylpiperidin-4-yl)-N-(4- (benzyloxy)-2-methylphenyl)-4- chloro-1H-pyrazole-5-carboxamide | 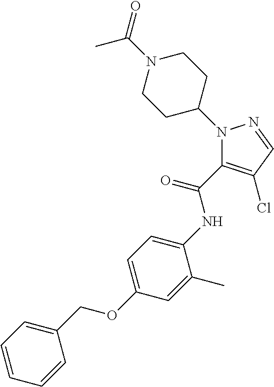 [SMILES: CC(=O)N1CCC(n2ncc(Cl)c2C(=O)Nc2ccc(OCc3ccccc3)cc2C)CC1] | 467.3 | 1.05 |
| 389 | 4-chloro-1-((1- cyanocyclopropyl)methyl)-N-(5-(1-(4- fluorophenyl)-1H-pyrazol-4-yl)-3- methylpyridin-2-yl)-1H-pyrazole-5- carboxamide | 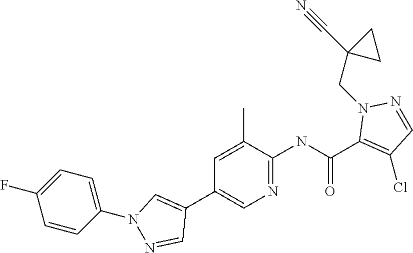 [SMILES: Cc1cc(-c2cnn(-c3ccc(F)cc3)c2)cnc1NC(=O)c1c(Cl)cnn1CC1(C#N)CC1] | 476 | 1.21 |
| 390 | tert-butyl 4-(4-chloro-5-((5-(3- isopropyl-4-methoxyphenyl)-3- methylpyridin-2-yl)carbamoyl)-1H- pyrazol-1-yl)piperidine-1-carboxylate | 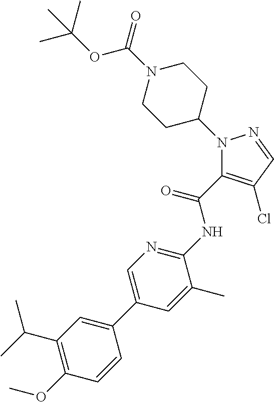 [SMILES: COc1ccc(-c2cnc(NC(=O)c3c(Cl)cnn3C3CCN(C(=O)OC(C)(C)C)CC3)c(C)c2)cc1C(C)C] | 568.4 | 1.21 |
| 391 | 1-(1-(1,4-dioxane-2- carbonyl)piperidin-4-yl)-N-(4- (benzyloxy)-2-methylphenyl)-4- chloro-1H-pyrazole-5-carboxamide | 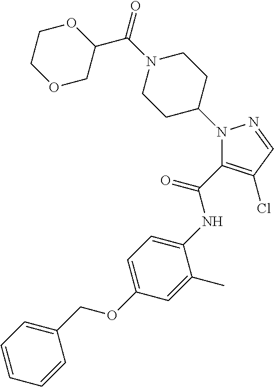 [SMILES: Cc1cc(OCc2ccccc2)ccc1NC(=O)c1c(Cl)cnn1C1CCN(C(=O)C2COCCO2)CC1] | 539.4 | 1.25 |
| 392 | N-(4-(benzyloxy)-2-methylphenyl)-4- chloro-1-(cis-4- propionamidocyclohexyl)-1H- pyrazole-5-carboxamide | 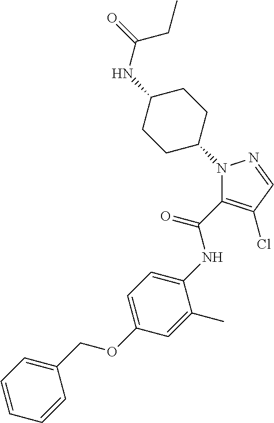 [SMILES: CCC(=O)N[C@H]1CC[C@@H](n2ncc(Cl)c2C(=O)Nc2ccc(OCc3ccccc3)cc2C)CC1] | 495.2 | 1.26 |
| 393 | 1-(1-acetylpiperidin-4-yl)-N-(5- (benzyloxy)-3-methylpyridin-2-yl)-4- chloro-1H-pyrazole-5-carboxamide | 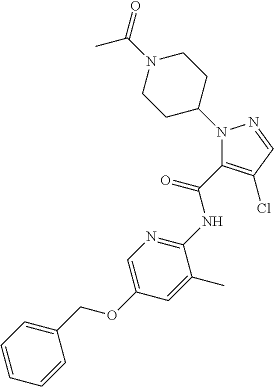 [SMILES: CC(=O)N1CCC(n2ncc(Cl)c2C(=O)Nc2ncc(OCc3ccccc3)cc2C)CC1] | 468.4 | 1.27 |
| 394 | 4-chloro-1-(3-cyanocyclobutyl)-N-(5- (1-(4-fluorophenyl)-1H-pyrazol-4-yl)- 3-methylpyridin-2-yl)-1H- pyrazole-5-carboxamide | 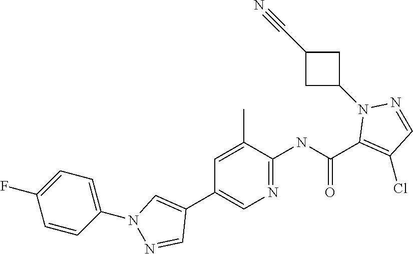 [SMILES: Cc1cc(-c2cnn(-c3ccc(F)cc3)c2)cnc1NC(=O)c1c(Cl)cnn1C1CC(C#N)C1] | 476 | 1.34 |
| 395 | (S)-4-chloro-N-(5-(1-(4-fluorophenyl)- 1H-pyrazol-4-yl)-3-methylpyridin-2- yl)-1-(tetrahydrofuran-3-yl)-1H- pyrazole-5-carboxamide | 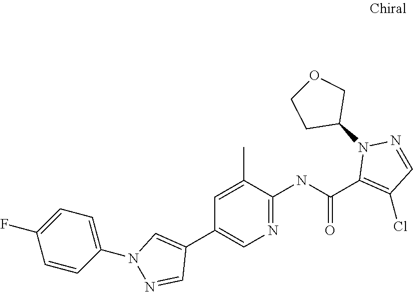 [SMILES: Cc1cc(-c2cnn(-c3ccc(F)cc3)c2)cnc1NC(=O)c1c(Cl)cnn1[C@H]1CCOC1] | 467 | 1.42 |
| 396 | tert-butyl 4-(4-chloro-5-((5-methyl-6′- (trifluoromethyl)-[3,3′-bipyridin]-6- yl)carbamoyl)-1H-pyrazol-1- yl)piperidine-1-carboxylate | 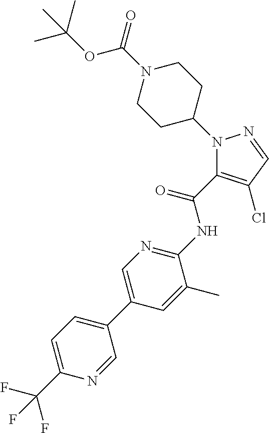 [SMILES: Cc1cc(-c2ccc(C(F)(F)F)nc2)cnc1NC(=O)c1c(Cl)cnn1C1CCN(C(=O)OC(C)(C)C)CC1] | 565.3 | 1.6 |
| 397 | N-(4-(benzyloxy)-2-methylphenyl)-4- chloro-1-(1-propionylpiperidin-4-yl)- 1H-pyrazole-5-carboxamide | 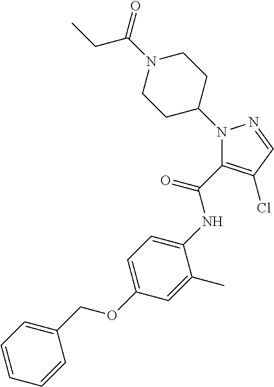 [SMILES: CCC(=O)N1CCC(n2ncc(Cl)c2C(=O)Nc2ccc(OCc3ccccc3)cc2C)CC1] | 481.5 | 1.68 |
| 398 | N-(4-(benzyloxy)-2-methylphenyl)-4- chloro-1-(2-isobutyryl-2- azaspiro[3.3]heptan-6-yl)-1H- pyrazole-5-carboxamide | 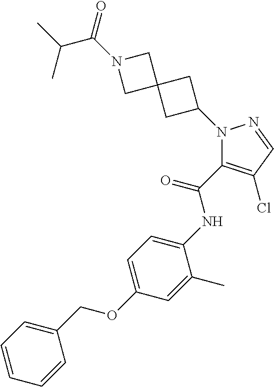 [SMILES: Cc1cc(OCc2ccccc2)ccc1NC(=O)c1c(Cl)cnn1C1CC2(C1)CN(C(=O)C(C)C)C2] | 507.2 | 1.74 |
| 399 | 1-(cis-4-acetamidocyclohexyl)-N-(4- (benzyloxy)-2-methylphenyl)-4- chloro-1H-pyrazole-5-carboxamide | 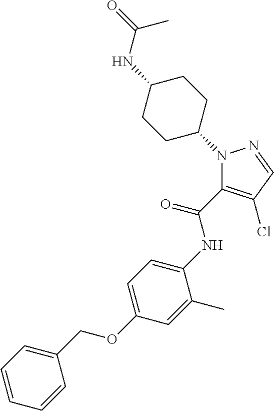 [SMILES: CC(=O)N[C@H]1CC[C@@H](n2ncc(Cl)c2C(=O)Nc2ccc(OCc3ccccc3)cc2C)CC1] | 481.5 | 1.76 |
| 400 | N-(4-(benzyloxy)-2-methylphenyl)-4- chloro-1-(1- (cyclopropanecarbonyl)piperidin-4- yl)-1H-pyrazole-5-carboxamide | 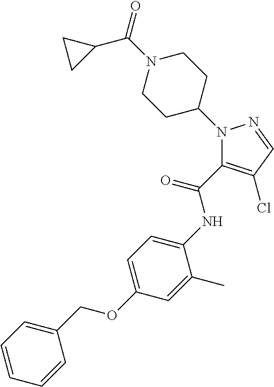 [SMILES: Cc1cc(OCc2ccccc2)ccc1NC(=O)c1c(Cl)cnn1C1CCN(C(=O)C2CC2)CC1] | 493.4 | 1.78 |
| 401 | N-(4-(benzyloxy)-2-methylphenyl)-4- chloro-1-(1-(3- methylbutanoyl)piperidin-4-yl)-1H- pyrazole-5-carboxamide | 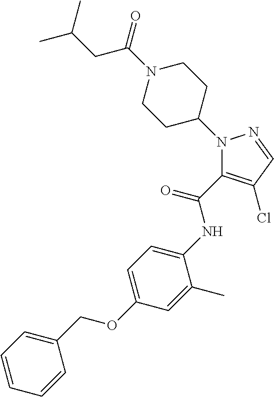 [SMILES: Cc1cc(OCc2ccccc2)ccc1NC(=O)c1c(Cl)cnn1C1CCN(C(=O)CC(C)C)CC1] | 509.2 | 1.78 |
| 402 | N-(4-(benzyloxy)-2-methylphenyl)-4- chloro-1-(1- (cyclobutanecarbonyl)piperidin-4-yl)- 1H-pyrazole-5-carboxamide | 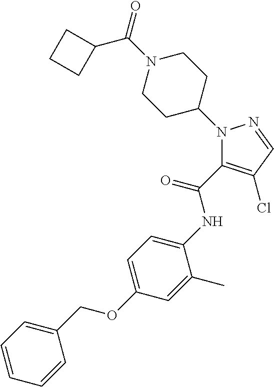 [SMILES: Cc1cc(OCc2ccccc2)ccc1NC(=O)c1c(Cl)cnn1C1CCN(C(=O)C2CCC2)CC1] | 507.4 | 2.16 |
| 403 | tert-butyl 4-(4-chloro-5-((5-(4- methoxy-2-methylphenyl)-3- methylpyridin-2-yl)carbamoyl)-1H- pyrazol-1-yl)piperidine-1-carboxylate | 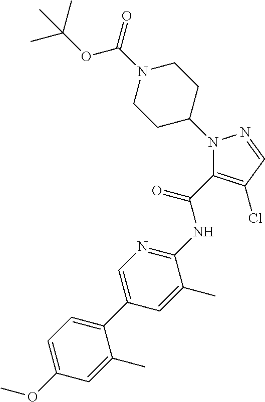 [SMILES: COc1ccc(-c2cnc(NC(=O)c3c(Cl)cnn3C3CCN(C(=O)OC(C)(C)C)CC3)c(C)c2)c(C)c1] | 540.4 | 2.29 |
| 404 | tert-butyl (cis-4-(5-((4-(benzyloxy)-2- methylphenyl)carbamoyl)-4-chloro- 1H-pyrazol-1-yl)cyclohexyl)carbamate | 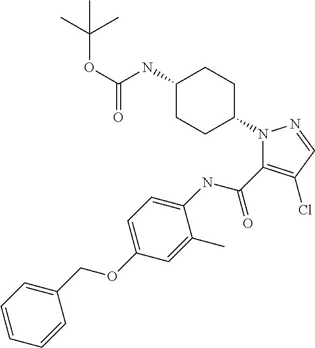 [SMILES: Cc1cc(OCc2ccccc2)ccc1NC(=O)c1c(Cl)cnn1[C@H]1CC[C@@H](NC(=O)OC(C)(C)C)CC1] | 439.2 (M − Boc + 2) | 2.30 |
| 405 | N-(4-(benzyloxy)-2-methylphenyl)-4- chloro-1-(1-pivaloylpiperidin-4-yl)- 1H-pyrazole-5-carboxamide | 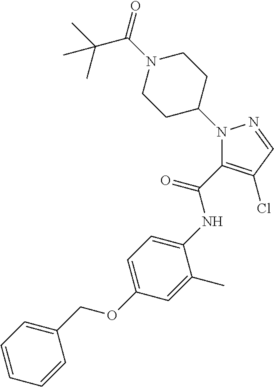 [SMILES: Cc1cc(OCc2ccccc2)ccc1NC(=O)c1c(Cl)cnn1C1CCN(C(=O)C(C)(C)C)CC1] | 509.4 | 2.31 |
| 406 | N-(4-(benzyloxy)-2-methylphenyl)-4- chloro-1-(2-(cyclopropanecarbonyl)-2- azaspiro[3.3]heptan-6-yl)-1H- pyrazole-5-carboxamide | 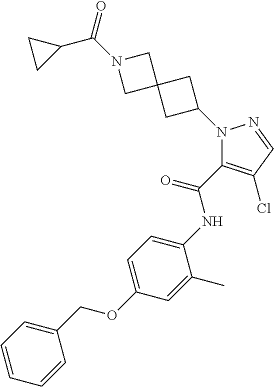 [SMILES: Cc1cc(OCc2ccccc2)ccc1NC(=O)c1c(Cl)cnn1C1CC2(C1)CN(C(=O)C1CC1)C2] | 505.2 | 2.54 |
| 407 | tert-butyl 4-(4-chloro-5-((6′-methoxy- 5-methyl-[3,3′-bipyridin]-6- yl)carbamoyl)-1H-pyrazol-1- yl)piperidine-1-carboxylate | 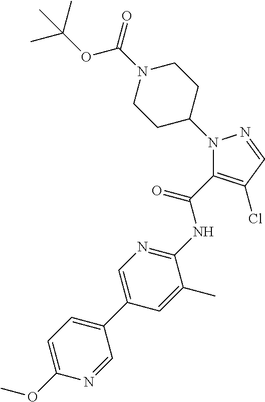 [SMILES: COc1ccc(-c2cnc(NC(=O)c3c(Cl)cnn3C3CCN(C(=O)OC(C)(C)C)CC3)c(C)c2)cn1] | 527.04 | 2.57 |
| 408 | tert-butyl 4-(4-chloro-5-((5-(3-ethyl-4- methoxyphenyl)-3-methylpyridin-2- yl)carbamoyl)-1H-pyrazol-1- yl)piperidine-1-carboxylate | 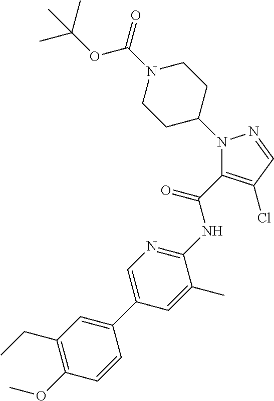 [SMILES: CCc1cc(-c2cnc(NC(=O)c3c(Cl)cnn3C3CCN(C(=O)OC(C)(C)C)CC3)c(C)c2)ccc1OC] | 554.4 | 2.67 |
| 409 | tert-butyl 4-(4-chloro-5-((3-methyl-5- (1-(p-tolyl)-1H-pyrazol-4-yl)pyridin- 2-yl)carbamoyl)-1H-pyrazol-1- yl)piperidine-1-carboxylate | 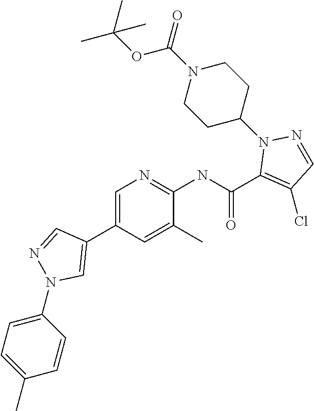 [SMILES: Cc1ccc(-n2cc(-c3cnc(NC(=O)c4c(Cl)cnn4C4CCN(C(=O)OC(C)(C)C)CC4)c(C)c3)cn2)cc1] | 576.4 | 2.77 |
| 410 | tert-butyl 4-(4-chloro-5-((3-methyl-5- (4-(thiophen-2-yl)phenyl)pyridin-2- yl)carbamoyl)-1H-pyrazol-1- yl)piperidine-1-carboxylate | 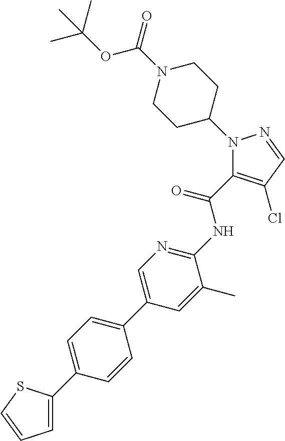 [SMILES: Cc1cc(-c2ccc(-c3cccs3)cc2)cnc1NC(=O)c1c(Cl)cnn1C1CCN(C(=O)OC(C)(C)C)CC1] | 578.2 | 2.78 |
| 411 | N-(4-(benzyloxy)-2-methylphenyl)-4- chloro-1-(1-isobutyrylpiperidin-4-yl)- 1H-pyrazole-5-carboxamide | 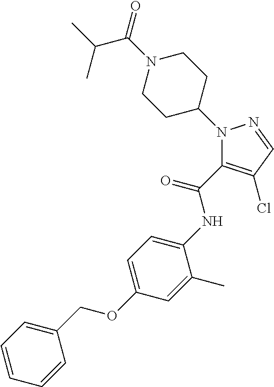 [SMILES: Cc1cc(OCc2ccccc2)ccc1NC(=O)c1c(Cl)cnn1C1CCN(C(=O)C(C)C)CC1] | 495.4 | 2.84 |
| 412 | 1-((4-(1,4-dioxane-2- carbonyl)morpholin-2-yl)methyl)-N- (4-(benzyloxy)-2-methylphenyl)-4- chloro-1H-pyrazole-5-carboxamide | 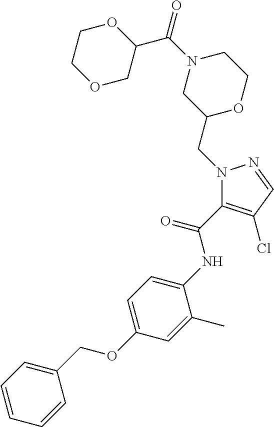 [SMILES: Cc1cc(OCc2ccccc2)ccc1NC(=O)c1c(Cl)cnn1CC1CN(C(=O)C2COCCO2)CCO1] | 555.2 | 3 |
| 413 | tert-butyl 4-(4-chloro-5-((3-methyl-5- (1-phenyl-1H-pyrazol-4-yl)pyridin-2- yl)carbamoyl)-1H-pyrazol-1- yl)piperidine-1-carboxylate | 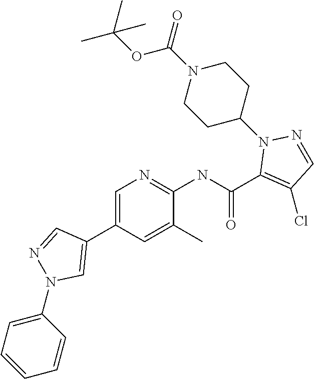 [SMILES: Cc1cc(-c2cnn(-c3ccccc3)c2)cnc1NC(=O)c1c(Cl)cnn1C1CCN(C(=O)OC(C)(C)C)CC1] | 562.4 | 3.53 |
| 414 | 1-(1-acetylpiperidin-4-yl)-4-chloro-N- (3-methyl-5-(4- (trifluoromethoxy)phenyl)pyridin-2- yl)-1H-pyrazole-5-carboxamide | 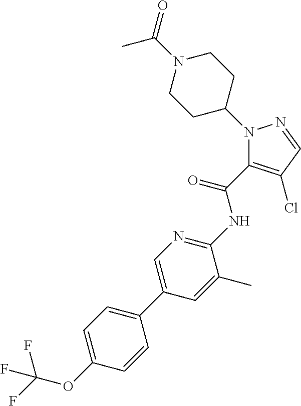 [SMILES: CC(=O)N1CCC(n2ncc(Cl)c2C(=O)Nc2ncc(-c3ccc(OC(F)(F)F)cc3)cc2C)CC1] | 522.2 | 3.62 |
| 415 | N-(4-(benzyloxy)-2-methylphenyl)-4- chloro-1-((4-pivaloylmorpholin-2- yl)methyl)-1H-pyrazole-5- carboxamide | 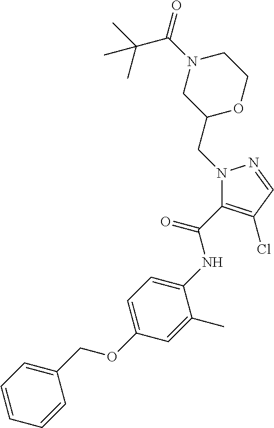 [SMILES: Cc1cc(OCc2ccccc2)ccc1NC(=O)c1c(Cl)cnn1CC1CN(C(=O)C(C)(C)C)CCO1] | 525.2 | 3.98 |
| 416 | 1-(1-acetylpiperidin-4-yl)-N-(5-(4- butoxyphenyl)-3-methylpyridin-2-yl)- 4-chloro-1H-pyrazole-5-carboxamide | 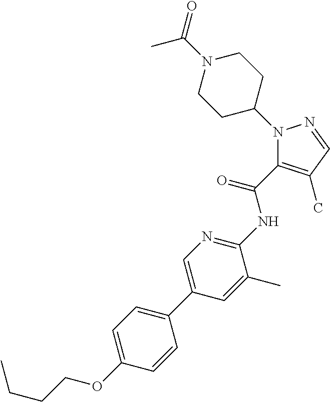 [SMILES: CCCCOc1ccc(-c2cnc(NC(=O)c3c(C)cnn3C3CCN(C(C)=O)CC3)c(C)c2)cc1] | 510.4 | 4.07 |
| 417 | N-(4-(benzyloxy)-2-methylphenyl)-4- chloro-1-((4-(2-methylcyclopropane- 1-carbonyl)morpholin-2-yl)methyl)- 1H-pyrazole-5-carboxamide | 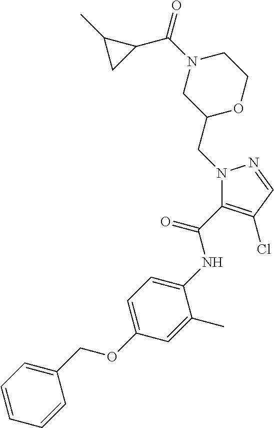 [SMILES: Cc1cc(OCc2ccccc2)ccc1NC(=O)c1c(Cl)cnn1CC1CN(C(=O)C2CC2C)CCO1] | 523.2 | 4.16 |
| 418 | 1-(1-acetylpiperidin-4-yl)-4-chloro-N- (5-(4-isobutylphenyl)-3- methylpyridin-2-yl)-1H-pyrazole-5- carboxamide | 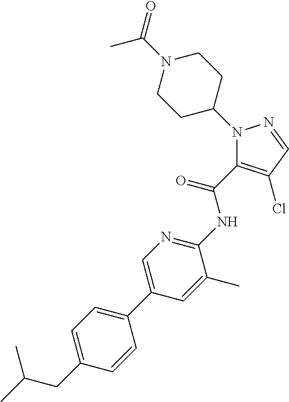 [SMILES: CC(=O)N1CCC(n2ncc(Cl)c2C(=O)Nc2ncc(-c3ccc(CC(C)C)cc3)cc2C)CC1] | 494.4 | 4.17 |
| 419 | tert-butyl 4-(4-chloro-5-((5-(2- methoxypyrimidin-5-yl)-3- methylpyridin-2-yl)carbamoyl)-1H- pyrazol-1-yl)piperidine-1-carboxylate | 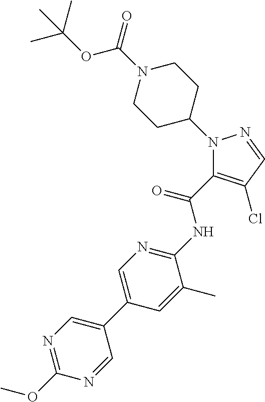 [SMILES: COc1ncc(-c2cnc(NC(=O)c3c(Cl)cnn3C3CCN(C(=O)OC(C)(C)C)CC3)c(C)c2)cn1] | 528.2 | 4.19 |
| 420 | N-(4-(benzyloxy)-2-methylphenyl)-4- chloro-1-((4-isobutyrylmorpholin-2- yl)methyl)-1H-pyrazole-5- carboxamide | 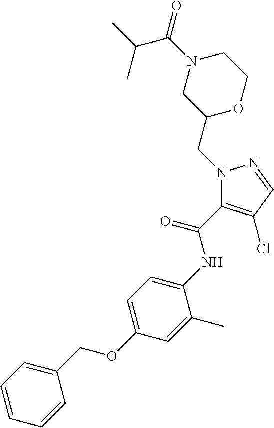 [SMILES: Cc1cc(OCc2ccccc2)ccc1NC(=O)c1c(Cl)cnn1CC1CN(C(=O)C(C)C)CCO1] | 511.2 | 4.38 |
| 421 | 1-(1-acetylpiperidin-4-yl)-4-chloro-N- (5-(4-(difluoromethoxy)phenyl)-3- methylpyridin-2-yl)-1H- pyrazole-5-carboxamide | 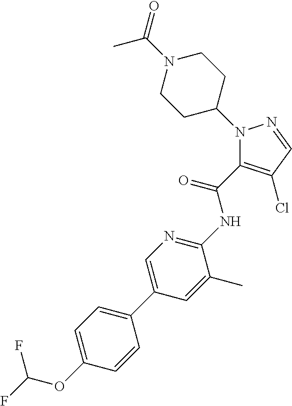 [SMILES: CC(=O)N1CCC(n2ncc(Cl)c2C(=O)Nc2ncc(-c3ccc(OC(F)F)cc3)cc2C)CC1] | 504.2 | 4.4 |
| 422 | 1-(1-acetylpiperidin-4-yl)-4-chloro-N- (3-methyl-5-(quinolin-3-yl)pyridin-2- yl)-1H-pyrazole-5-carboxamide | 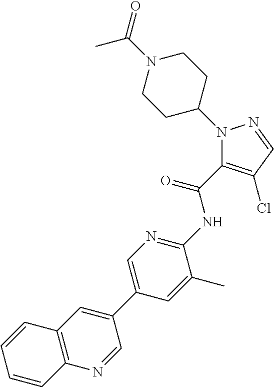 [SMILES: CC(=O)N1CCC(n2ncc(Cl)c2C(=O)Nc2ncc(-c3cnc4ccccc4c3)cc2C)CC1] | 489.3 | 4.46 |
| 423 | 1-((4-acetylmorpholin-2-yl)methyl)-N- (4-(benzyloxy)-2-methylphenyl)-4- chloro-1H-pyrazole-5-carboxamide | 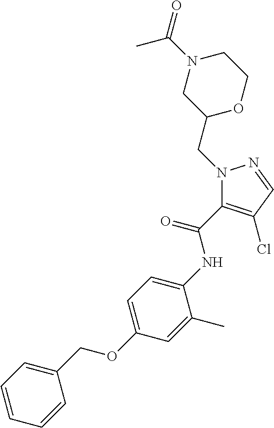 [SMILES: CC(=O)N1CCOC(Cn2ncc(Cl)c2C(=O)Nc2ccc(OCc3ccccc3)cc2C)C1] | 483.2 | 4.82 |
| 424 | N-(4-(benzyloxy)-2-methylphenyl)-4- chloro-1-((4- (cyclobutanecarbonyl)morpholin-2- yl)methyl)-1H- pyrazole-5-carboxamide | 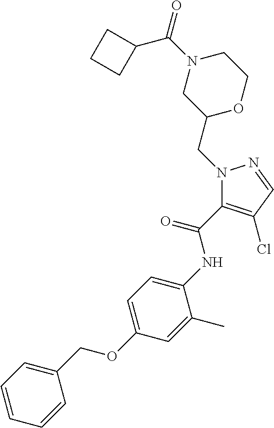 [SMILES: Cc1cc(OCc2ccccc2)ccc1NC(=O)c1c(Cl)cnn1CC1CN(C(=O)C2CCC2)CCO1] | 523.3 | 4.85 |
| 425 | 1-(1-acetylpiperidin-4-yl)-4-chloro-N- (5′-chloro-6′-methoxy-5-methyl-[3,3′- bipyridin]-6-yl)-1H- pyrazole-5-carboxamide | 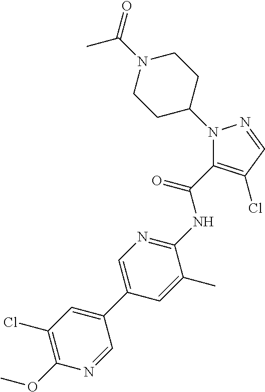 [SMILES: COc1ncc(-c2cnc(NC(=O)c3c(Cl)cnn3C3CCN(C(C)=O)CC3)c(C)c2)cc1Cl] | 503.2 | 5.05 |
| 426 | 1-(1-acetylpiperidin-4-yl)-4-chloro-N- (5-(4-chloro-3,5-difluorophenyl)-3- methylpyridin-2-yl)-1H- pyrazole-5-carboxamide | 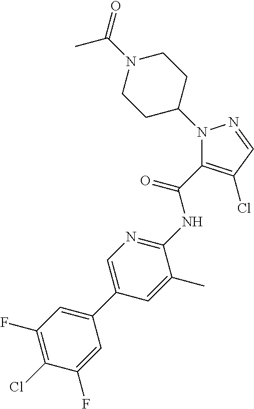 [SMILES: CC(=O)N1CCC(n2ncc(Cl)c2C(=O)Nc2ncc(-c3cc(F)c(Cl)c(F)c3)cc2C)CC1] | 508.2 | 5.12 |
| 427 | 1-(1-acetylpiperidin-4-yl)-4-chloro-N- (3-methyl-5-(4- (trifluoromethyl)phenyl)pyridin-2-yl)- 1H-pyrazole-5-carboxamide | 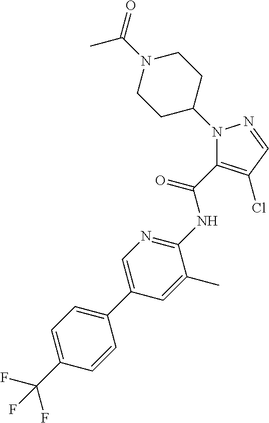 [SMILES: CC(=O)N1CCC(n2ncc(Cl)c2C(=O)Nc2ncc(-c3ccc(C(F)(F)F)cc3)cc2C)CC1] | 506.3 | 5.21 |
| 428 | 1-(1-acetylpiperidin-4-yl)-4-chloro-N- (5-(3,5-difluoro-4-methoxyphenyl)-3- methylpyridin-2-yl)-1H- pyrazole-5-carboxamide | 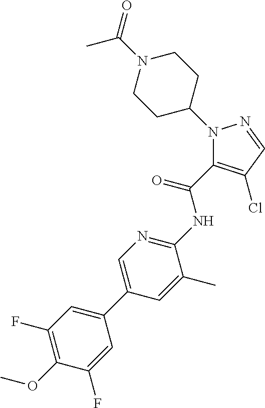 [SMILES: COc1c(F)cc(-c2cnc(NC(=O)c3c(Cl)cnn3C3CCN(C(C)=O)CC3)c(C)c2)cc1F] | 504.3 | 5.31 |
| 429 | N-(4-(benzyloxy)-2-methylphenyl)-4- chloro-1-((4-(3- methylbutanoyl)morpholin-2- yl)methyl)-1H- pyrazole-5-carboxamide | 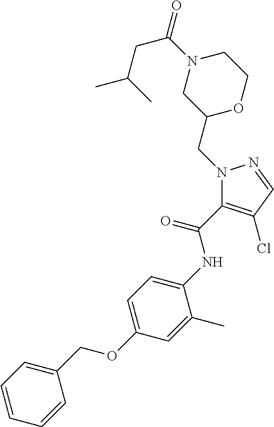 [SMILES: Cc1cc(OCc2ccccc2)ccc1NC(=O)c1c(Cl)cnn1CC1CN(C(=O)CC(C)C)CCO1] | 525.4 | 5.52 |
| 430 | tert-butyl 4-(5-((4-(benzyloxy)-2- methylphenyl)carbamoyl)-4-chloro- 1H-pyrazol-1-yl)piperidine-1- carboxylate | 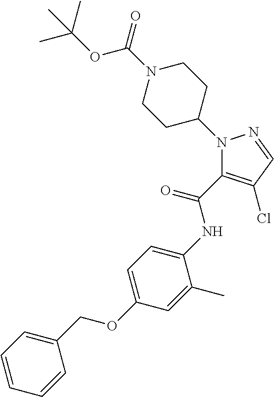 [SMILES: Cc1cc(OCc2ccccc2)ccc1NC(=O)c1c(Cl)cnn1C1CCN(C(=O)OC(C)(C)C)CC1] | 425.3 (M − Boc + 2) | 6.35 |
| 431 | tert-butyl 4-(4-chloro-5-((5-(4- ethoxyphenyl)-3-methylpyridin-2- yl)carbamoyl)-1H-pyrazol-1- yl)piperidine-1-carboxylate | 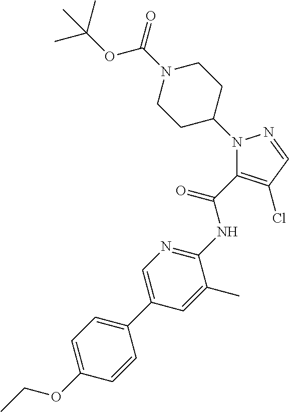 [SMILES: CCOc1ccc(-c2cnc(NC(=O)c3c(Cl)cnn3C3CCN(C(=O)OC(C)(C)C)CC3)c(C)c2)cc1] | 540.4 | 6.4 |
| 432 | N-(4-(benzyloxy)-2-methylphenyl)-4- chloro-1-((4- (cyclopropanecarbonyl)morpholin-2- yl)methyl)-1H- pyrazole-5-carboxamide | 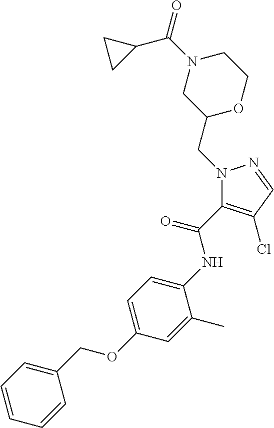 [SMILES: Cc1cc(OCc2ccccc2)ccc1NC(=O)c1c(Cl)cnn1CC1CN(C(=O)C2CC2)CCO1] | 509.2 | 6.45 |
| 433 | 1-(2-acetyl-2-azaspiro[3.3]heptan-6- yl)-N-(4-(benzyloxy)-2- methylphenyl)-4-chloro-1H-pyrazole- 5-carboxamide | 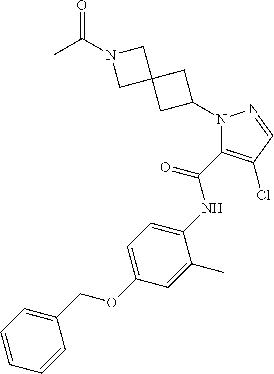 [SMILES: CC(=O)N1CC2(CC(n3ncc(Cl)c3C(=O)Nc3ccc(OCc4ccccc4)cc3C)C2)C1] | 479.2 | 6.89 |
| 434 | 1-(1-acetylpiperidin-4-yl)-4-chloro-N- (5-(3-chloro-4-methoxyphenyl)-3- methylpyridin-2-yl)-1H-pyrazole-5- carboxamide | 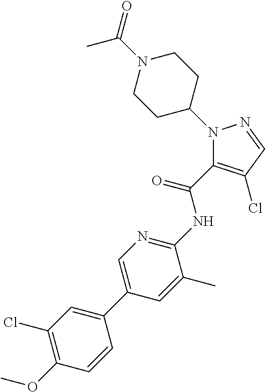 [SMILES: COc1ccc(-c2cnc(NC(=O)c3c(Cl)cnn3C3CCN(C(C)=O)CC3)c(C)c2)cc1Cl] | 502.2 | 7.43 |
| 435 | N-(4-benzyloxy)-2-methylphenyl)-4- chloro-1-((4-propionylmorpholin-2- yl)methyl)-1H- pyrazole-5-carboxamide | 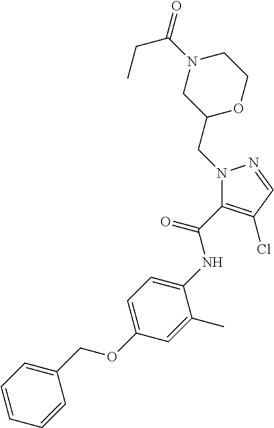 [SMILES: CCC(=O)N1CCOC(Cn2ncc(Cl)c2C(=O)Nc2ccc(OCc3ccccc3)cc2C)C1] | 497.3 | 8.11 |
| 436 | tert-butyl 4-(4-chloro-5-((5-(1-(2- fluorophenyl)-1H-pyrazol-4-yl)-3- methylpyridin-2-yl)carbamoyl)-1H- pyrazol-1-yl)piperidine-1-carboxylate | 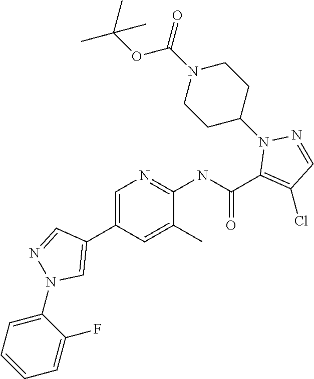 [SMILES: Cc1cc(-c2cnn(-c3ccccc3F)c2)cnc1NC(=O)c1c(Cl)cnn1C1CCN(C(=O)OC(C)(C)C)CC1] | 580.4 | 9.30 |
| 437 | tert-butyl 4-(4-chloro-5-((2′-methoxy- 5-methyl-[3,4′-bipyridin]-6- yl)carbamoyl)-1H-pyrazol-1- yl)piperidine-1-carboxylate | 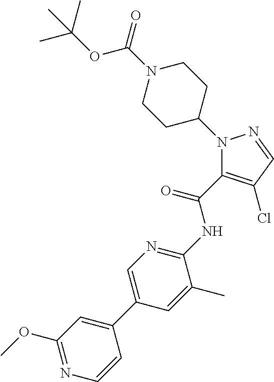 [SMILES: COc1cc(-c2cnc(NC(=O)c3c(Cl)cnn3C3CCN(C(=O)OC(C)(C)C)CC3)c(C)c2)ccn1] | 527.2 | 9.96 |
| 454 | 4-chloro-1-[(3S)-1- (cyclopropanecarbonyl)pyrrolidin-3- yl]-N-[3-methyl-5- (phenylethynyl)pyridin-2-yl]-1H- pyrazole-5-carboxamide | 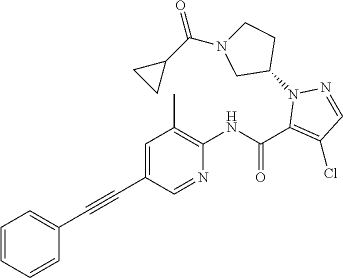 [SMILES: Cc1cc(C#Cc2ccccc2)cnc1NC(=O)c1c(Cl)cnn1[C@H]1CCN(C(=O)C2CC2)C1] | 474.2*¹ | 0.13*¹² |
| 455 | 4-chloro-1-[(3R)-1- (cyclopropanecarbonyl)pyrrolidin-3- yl]-N-[3-methyl-5- (phenylethynyl)pyridin-2-yl]-1H- pyrazole-5-carboxamide | 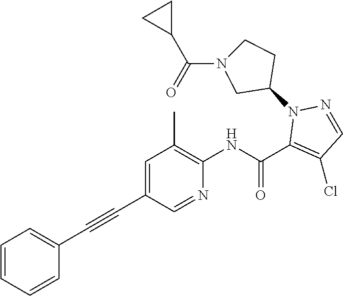 [SMILES: Cc1cc(C#Cc2ccccc2)cnc1NC(=O)c1c(Cl)cnn1[C@@H]1CCN(C(=O)C2CC2)C1] | 474.2*¹ | 0.14*¹² |
| 456*¹⁰ | 4-chloro-N-[3-methyl-5- (phenylethynyl)pyridin-2-yl]-1-[(5S)- 4,5,6,7-tetrahydropyrazolo[1,5- a]pyridin-5-yl]-1H-pyrazole-5- carboxamide or 4-chloro-N-[3-methyl-5- (phenylethynyl)pyridin-2-yl]-1-[(5R)- 4,5,6,7-tetrahydropyrazolo[1,5- a]pyridin-5-yl]-1H-pyrazole-5- carboxamide | 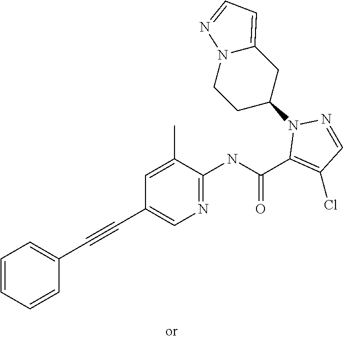 [SMILES: Cc1cc(C#Cc2ccccc2)cnc1NC(=O)c1c(Cl)cnn1[C@H]1CCn2nccc2C1] | 457.0*¹³ | 0.12*¹² |
| | | 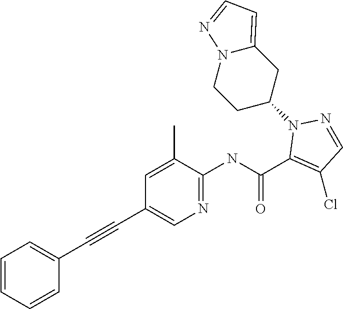 [SMILES: Cc1cc(C#Cc2ccccc2)cnc1NC(=O)c1c(Cl)cnn1[C@@H]1CCn2nccc2C1] | | |
| 457*¹¹ | 4-chloro-N-[3-methyl-5- (phenylethynyl)pyridin-2-yl]-1-[(5S)- 4,5,6,7-tetrahydropyrazolo[1,5- a]pyridin-5-yl]-1H-pyrazole-5- carboxamide or 4-chloro-N-[3-methyl-5- (phenylethynyl)pyridin-2-yl]-1-[(5R)- 4,5,6,7-tetrahydropyrazolo[1,5- a]pyridin-5-yl]-1H-pyrazole-5- carboxamide | 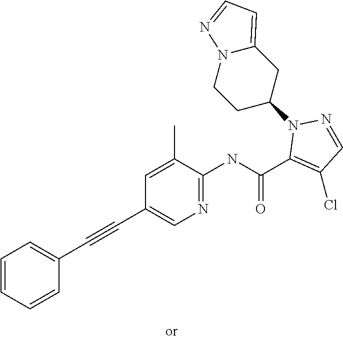 [SMILES: Cc1cc(C#Cc2ccccc2)cnc1NC(=O)c1c(Cl)cnn1[C@H]1CCn2nccc2C1] | 457.0*¹³ | 0.037*¹² |
| | | 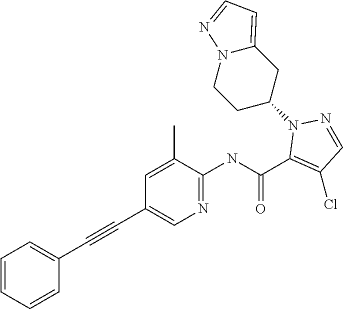 [SMILES: Cc1cc(C#Cc2ccccc2)cnc1NC(=O)c1c(Cl)cnn1[C@@H]1CCn2nccc2C1] | | |

*¹The data were measured by Reversed-phase LCMS method (3) as described above.
*²Peak to elute (analyzed by SFC) Retention Time 3.35 min, Column: Lux Cellulose; Column dimensions: 20 × 250 mm; Co-solvent: MeOH; Gradient Profile: 30%
isocratic; Flow rate: 80 mL/min
*³Peak to elute (analyzed by SFC) Retention Time 8.54 min Column: Lux Cellulose; Column dimensions: 20 × 250 mm; Co-solvent: MeOH; Gradient Profile: 30%
isocratic; Flow rate: 80 mL/min
*⁴Peak of Boc intermediate (the intermediate corresponding to D21) in synthesizing of 114: to elute (analyzed by SFC) Retention Time 5.6 min, Column: Chiralpak IA;
Column dimensions: 20 × 250 mm; Co-solvent: MeOH; Gradient Profile: 20% isocratic; Flow rate: 80 mL/min
*⁵Peak of Boc intermediate (the intermediate corresponding to D21) in synthesizing of 31: to elute (analyzed by SFC) Retention Time 6.56 min, Column: Chiralpak IA;
Column dimensions: 20 × 250 mm, Co-solvent: MeOH; Gradient Profile: 20% isocratic; Flow rate: 80 mL/min
*⁶Peak of Boc intermediate (the intermediate corresponding to D21) in synthesizing of 261: peak to elute (anlayzed by SFC) Retention Time 8.09 min, Column:
Chiralpak IA; Column dimensions: 20 × 250 mm; Co-solvent: MeOH; Gradient Profile: 20% isocratic; Flow rate: 80 mL/min
*⁷Peak of Boc intermediate (the intermediate corresponding to D21) in synthesizing of 86: peak to elute (analyzed by SFC) Retention Time 18.43 min, Column:
Chiralpak IA; Column dimensions: 20 × 250 mm; Co-solvent: MeOH; Gradient Profile: 20% isocratic; Flow rate: 80 mL/min
*⁸Peak of carboxylic acid intermediate (the intermediate corresponding to carboxylic acid of C11) in synthesizing of 362: peak to elute (anlayzed by Reversed-
phase LCMS method (1)) Retention Time 0.902 min
*⁹Peak of carboxylic acid intermediate (the intermediate corresponding to carboxylic acid of C11) in synthesizing of 363: peak to elute (analyzed by Reversed-
phase LCMS method (1)) Retention Time 0.909 min
*¹⁰Peak to elute (analyzed by SFC) Retention Time 3.14 min, Column: Chiralpak AS-3 100 × 4 mm I.D., 3 um; Mobile phase: A: CO2, B: ethanol (0.05% DEA);
Gradient: from 5% to 40% of B in 4 min and hold 40% for 2.5 min, then 5% of B for 1.5 min; Flow rate: 2.8 mL/min, Column temp.: 35 degrees, ABPR: 1500psi
*¹¹Peak to elute (analyzed by SFC) Retention Time 3.49 min, Column: Chiralpak AS-3 100 × 4.6 mm I.D., 3 um; Mobile phase: A: CO2, B: ethanol (0.05%
DEA); Gradient from 5% to 40% of B in 4 min and hold 40% for 2.5 min, then 5% of B for 1.5 min; Flow rate: 2.8 mL/min, Column temp.: 35 degrees, ABPR:
1500psi
*¹²The data were measured by Method 2 of TREK1 Thalium flux assay as described above.
*¹³The data were measured by Reversed-phase LCMS method (2) as described above.

The compounds shown in Table 2 were prepared in an analogous manner with the appropriate starting materials, and evaluated in our TREK-1 Manual Patch Clamp (hMPC) assay.

TABLE 2

| | | | ES-MS | hMPC |
|---|---|---|---|---|
| Cpd. No. | Name | Structure | [M + 1]⁺ | Assay IC₅₀ (μM) |
| 1 | 1-((1,4-dioxan-2-yl)methyl)-4-chloro- N-(3-methyl-5-(phenylethynyl)pyridin- 2-yl)-1H-pyrazole-5-carboxamide | 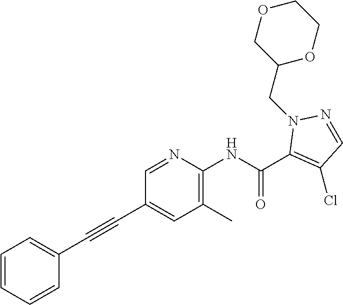 [SMILES: Cc1cc(C#Cc2ccccc2)cnc1NC(=O)c1c(Cl)cnn1CC1COCCO1] | 437 | 0.17 |
| 2 | 4-chloro-N-(3-methyl-5- (phenylethynyl)pyridin-2-yl)-1- ((tetrahydro-2H-pyran-4-yl)methyl)- 1H-pyrazole-5-carboxamide | 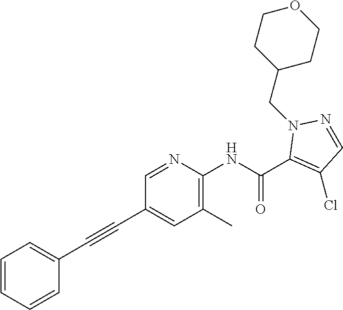 [SMILES: Cc1cc(C#Cc2ccccc2)cnc1NC(=O)c1c(Cl)cnn1CC1CCOCC1] | 435 | 0.06 |
| 3 | (S)-4-chloro-N-(3-methyl-5- (phenylethynyl)pyridin-2-yl)-1- (tetrahydrofuran-3-yl)-1H-pyrazole-5- carboxamide | 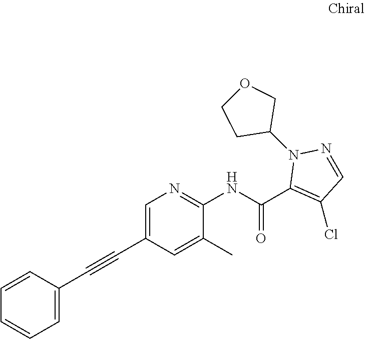 [SMILES: Cc1cc(C#Cc2ccccc2)cnc1NC(=O)c1c(Cl)cnn1C1CCOC1] | 407 | 0.53 |
| 4 | (S)-1-((1,4-dioxan-2-yl)methyl)-4- chloro-N-(3-fluoro-5- (phenylethynyl)pyridin-2-yl)-1H- pyrazole-5-carboxamide | 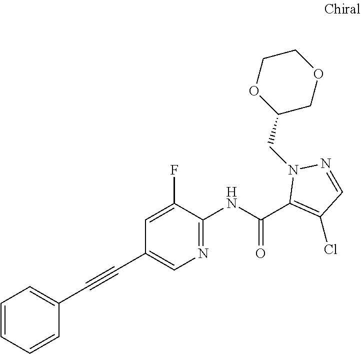 [SMILES: O=C(Nc1ncc(C#Cc2ccccc2)cc1F)c1c(Cl)cnn1C[C@H]1COCCO1] | 441 | 0.07 |
| 5 | 1-(1-acetylpiperidin-4-yl)-4-chloro-N- (3-methyl-5-(phenylethynyl)pyridin-2- yl)-1H-pyrazole-5-carboxamide | 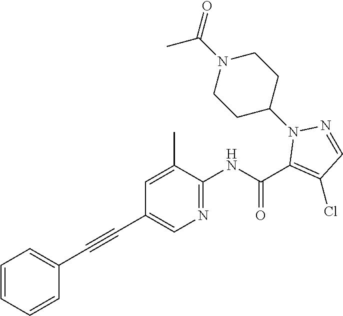 [SMILES: CC(=O)N1CCC(n2ncc(Cl)c2C(=O)Nc2ncc(C#Cc3ccccc3)cc2C)CC1] | 462 | 0.06 |
| 6 | 1-(1-acetylpiperidin-4-yl)-4-chloro-N- (3-fluoro-5-(phenylethynyl)pyridin-2- yl)-1H-pyrazole-5-carboxamide | 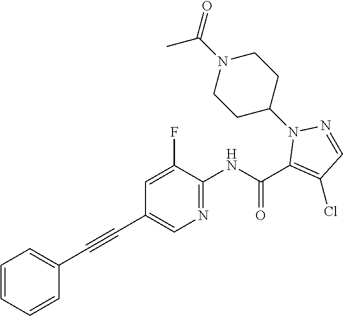 [SMILES: CC(=O)N1CCC(n2ncc(Cl)c2C(=O)Nc2ncc(C#Cc3ccccc3)cc2F)CC1] | 466 | 0.07 |
| 7 | 1-(1-acetylpiperidin-4-yl)-4-chloro-N- (3-fluoro-5-((4- fluorophenyl)ethynyl)pyridin-2-yl)-1H- pyrazole-5-carboxamide | 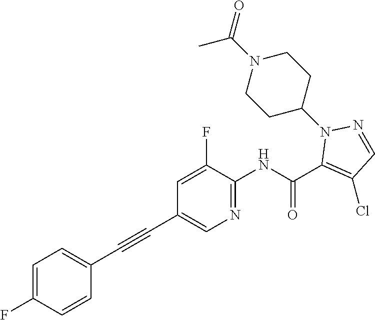 [SMILES: CC(=O)N1CCC(n2ncc(Cl)c2C(=O)Nc2ncc(C#Cc3ccc(F)cc3)cc2F)CC1] | 483.9 | 0.03 |
| 8 | 1-(1-acetylpiperidin-4-yl)-4-chloro-N- (5-((4-fluorophenyl)ethynyl)-3- methylpyridin-2-yl)-1H-pyrazole-5- carboxamide | 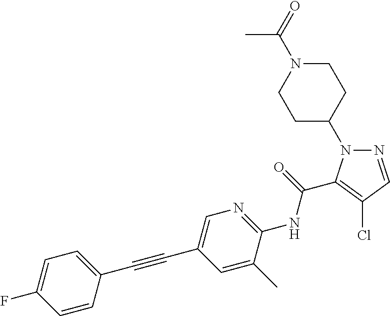 [SMILES: CC(=O)N1CCC(n2ncc(Cl)c2C(=O)Nc2ncc(C#Cc3ccc(F)cc3)cc2C)CC1] | 480.4 | 0.09 |
| 9 | 4-chloro-N-(3-methyl-5- (phenylethynyl)pyridin-2-yl)-1-(4,5,6,7- tetrahydropyrazolo[1,5-a]pyridin-5-yl)- 1H-pyrazole-5-carboxamide | 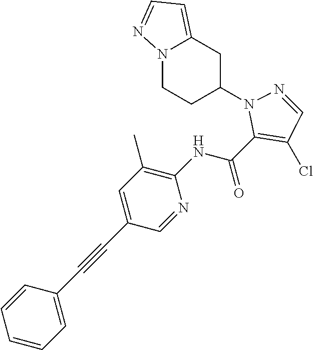 [SMILES: Cc1cc(C#Cc2ccccc2)cnc1NC(=O)c1c(Cl)cnn1C1CCn2nccc2C1] | 457 | 0.02 |
| 10 | 1-((3R,4R)-1-acetyl-3-fluoropiperidin- 4-yl)-4-chloro-N-(5-((4- fluorophenyl)ethynyl)-3-methylpyridin- 2-yl)-1H-pyrazole-5-carboxamide | 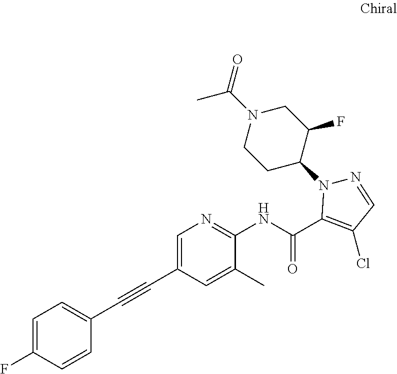 [SMILES: CC(=O)N1CC[C@H](n2ncc(Cl)c2C(=O)Nc2ncc(C#Cc3ccc(F)cc3)cc2C)[C@H](F)C1] | 498.3 | 0.06 |
| 11 | 1-(1-(1,4-dioxane-2-carbonyl)piperidin- 4-yl)-4-chloro-N-(3-fluoro-5- (phenylethynyl)pyridin-2-yl)-1H- pyrazole-5-carboxamide | 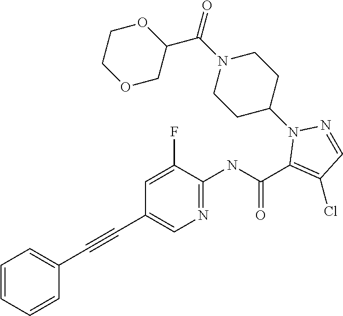 [SMILES: O=C(Nc1ncc(C#Cc2ccccc2)cc1F)c1c(Cl)cnn1C1CCN(C(=O)C2COCCO2)CC1] | 538 | 0.1 |
| 12 | 1-(cis-4-acetamidocyclohexyl)-4- chloro-N-(3-methyl-5- (phenylethynyl)pyridin-2-yl)-1H- pyrazole-5-carboxamide | 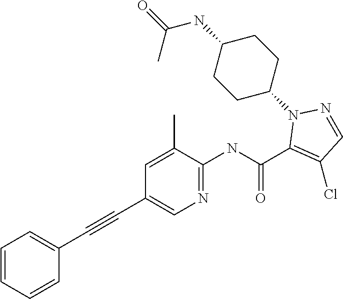 [SMILES: CC(=O)N[C@H]1CC[C@@H](n2ncc(Cl)c2C(=O)Nc2ncc(C#Cc3ccccc3)cc2C)CC1] | 476.3 | 0.06 |
| 13 | 1-(cis-4-acetamidocyclohexyl)-4- chloro-N-(3-fluoro-5- (phenylethynyl)pyridin-2-yl)-1H- pyrazole-5-carboxamide | 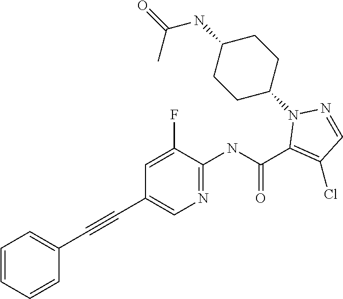 [SMILES: CC(=O)N[C@H]1CC[C@@H](n2ncc(Cl)c2C(=O)Nc2ncc(C#Cc3ccccc3)cc2F)CC1] | 480.3 | 0.06 |
| 14 | 1-(cis-4-acetamidocyclohexyl)-4- chloro-N-(5-((4-fluorophenyl)ethynyl)- 3-methylpyridin-2-yl)-1H-pyrazole-5- carboxamide | 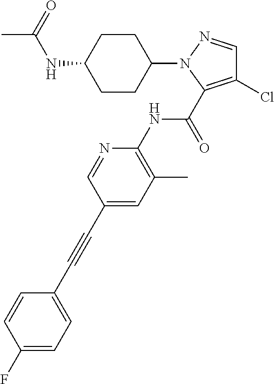 | 494.3 | 0.002 |
| 15 | 1-((1-acetyl-4-fluoropiperidin-4- yl)methyl)-4-chloro-N-(3-methyl-5- (phenylethynyl)pyridin-2-yl)-1H- pyrazole-5-carboxamide | 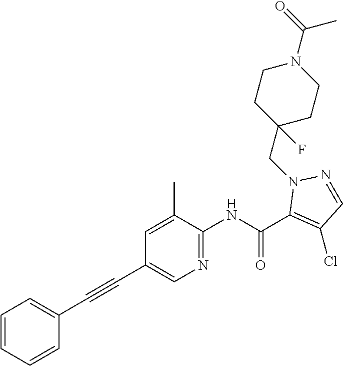 [SMILES: CC(=O)N1CCC(F)(Cn2ncc(Cl)c2C(=O)Nc2ncc(C#Cc3ccccc3)cc2C)CC1] | 494.3 | 0.29 |
| 16*² | 1-[(1R)-1-(1-acetylpiperidin-4- yl)ethyl]-4-chloro-N-[3-methyl-5- (phenylethynyl)pyridin-2-yl]-1H- pyrazole-5-carboxamide or 1-[(1S)-1-(1-acetylpiperidin-4-yl)ethyl]- 4-chloro-N-[3-methyl-5- (phenylethynyl)pyridin-2-yl]-1H- pyrazole-5-carboxamide | 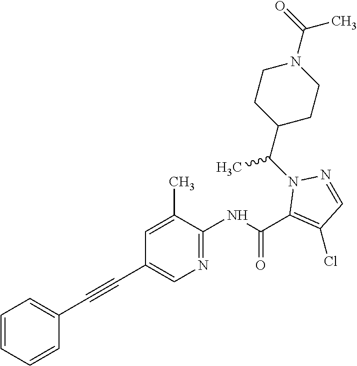 [SMILES: CC(=O)N1CCC(C(C)n2ncc(Cl)c2C(=O)Nc2ncc(C#Cc3ccccc3)cc2C)CC1] | 490.4 | 0.16 |
| 140 | 1-((1-acetyl-3-fluoroazetidin-3- yl)methyl)-4-chloro-N-(3-fluoro-5- (phenylethynyl)pyridin-2-yl)-1H- pyrazole-5-carboxamide | 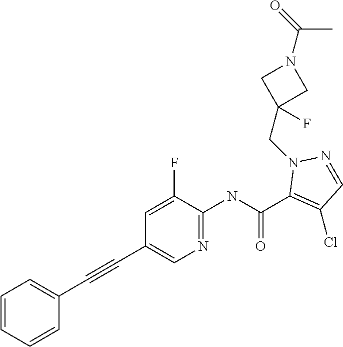 [SMILES: CC(=O)N1CC(F)(Cn2ncc(Cl)c2C(=O)Nc2ncc(C#Cc3ccccc3)cc2F)C1] | 470.3 | 0.13 |
| 215 | 1-((1-acetyl-3-fluoroazetidin-3- yl)methyl)-4-chloro-N-(3-methyl-5- (phenylethynyl)pyridin-2-yl)-1H- pyrazole-5-carboxamide | 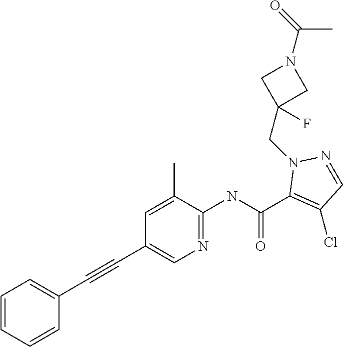 [SMILES: CC(=O)N1CC(F)(Cn2ncc(Cl)c2C(=O)Nc2ncc(C#Cc3ccccc3)cc2C)C1] | 466.3 | 0.13 |
| 454 | 4-chloro-1-[(3S)-1- (cyclopropanecarbonyl)pyrrolidin-3-yl]- N-[3-methyl-5-(phenylethynyl)pyridin- 2-yl]-1H-pyrazole-5-carboxamide | 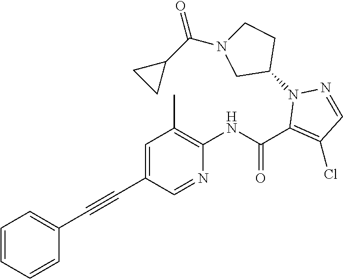 [SMILES: Cc1cc(C#Cc2ccccc2)cnc1NC(=O)c1c(Cl)cnn1[C@H]1CCN(C(=O)C2CC2)C1] | 474*¹ | 0.10 |

*¹The data were measured by Reversed-phase LCMS method (3) as described above.
*²Peak to elute (analyzed by SFC) Retention Time 3.35 min, Column: Lux Cellulose; Column demensions: 20 × 250 mm; Co-solvent: MeOH; Gradient
Profile: 30% isocratic; Flow rate: 80 mL/min

The compounds shown in Table 3 were prepared in an analogous manner with the appropriate starting materials, and evaluated in our TREK-1 Thallium (Tl⁺) flux assay.

TABLE 3

| | | | | Tl⁺ Assay |
|---|---|---|---|---|
| | | | | % inhibition |
| | | | | at 30 uM |
| | | | | (Minus value |
| | | | | indicates |
| Cpd. | | | ES-MS | activation of |
| No. | Name | Structure | [M + 1]⁺ | TREK-1) |
| 438 | 5-amino-4-chloro-N-(3-methyl-5- (phenylethnyl)pyridin-2-yl)-1- ((tetrahydro-2H-pyran-4-yl)methyl)- 1H-pyrazole-3-carboxamide | 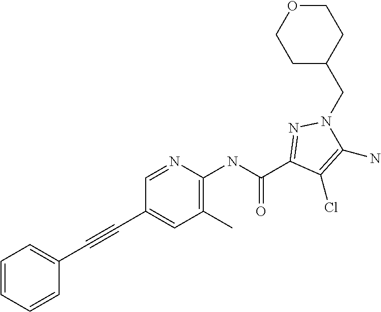 [SMILES: Cc1cc(C#Cc2ccccc2)cnc1NC(=O)c1nn(CC2CCOCC2)c(N)c1Cl] | 450 | −332 |
| 439 | 1-(2-(1-acetylpiperidin-4-yl)ethyl)- 4-chloro-N-(5-((3- fluorophenyl)ethynyl)-3- methylpyridin-2-yl)-1H-pyrazol-3- carboxamide | 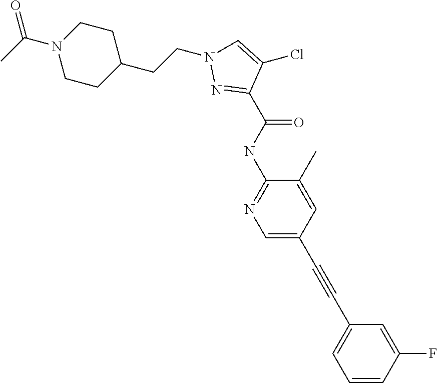 [SMILES: CC(=O)N1CCC(CCn2cc(Cl)c(C(=O)Nc3ncc(C#Cc4cccc(F)c4)cc3C)n2)CC1] | 508.4 | −163 |
| 440 | 1-(2-(1-actetylpiperidin-4-yl)ethyl)- 4-chloro-N-(3-methyl-5-(thiophen- 2-ylethynyl)pyridin-2-yl)-1H- pyrazole-3-carboxamide | 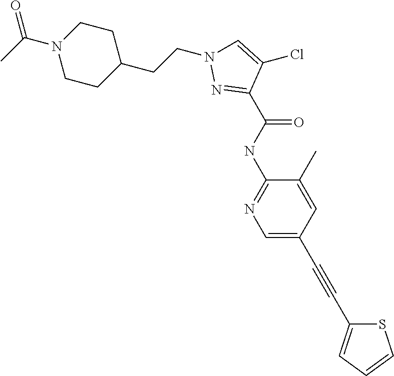 [SMILES: CC(=O)N1CCC(CCn2cc(Cl)c(C(=O)Nc3ncc(C#Cc4cccs4)cc3C)n2)CC1] | 496.3 | −135 |
| 441 | 4-chloro-1-(2-(1- (cyclopropanecarbonyl)piperidin-4- yl)ethyl)-N-(5-((2- fluorophenyl)ethynyl)-3- methylpyridin-2-yl)-1H-pyrazole-3- carboxamide | 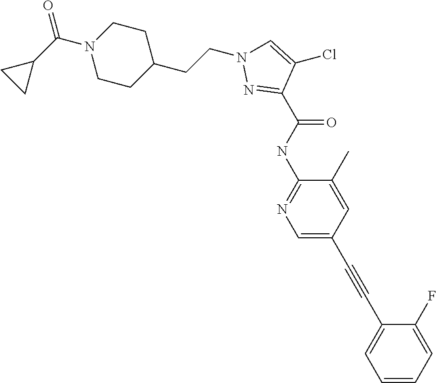 [SMILES: Cc1cc(C#Cc2ccccc2F)cnc1NC(=O)c1nn(CCC2CCN(C(=O)C3CC3)CC2)cc1Cl] | 534.4 | −123 |
| 442 | 1-(2-(1-acetylpiperidin-4-yl)ethyl)- 4-chloro-N-(5-((4- fluorophenyl)ethynyl)-3- methylpyridin-2-yl)-1H-pyrazole-3- carboxamide | 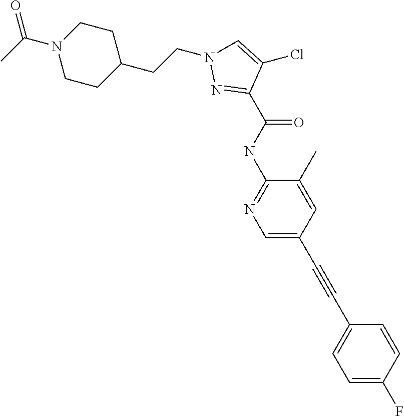 [SMILES: CC(=O)N1CCC(CCn2cc(Cl)c(C(=O)Nc3ncc(C#Cc4ccc(F)cc4)cc3C)n2)CC1] | 508.3 | −118 |
| 443 | 4-chloro-N-(3-methyl-5- (phenylethynyl)pyridin-2-yl)-1- (tetrahydro-2H-pyran-4-yl)-1H- pyrazole-3-carboxamide | 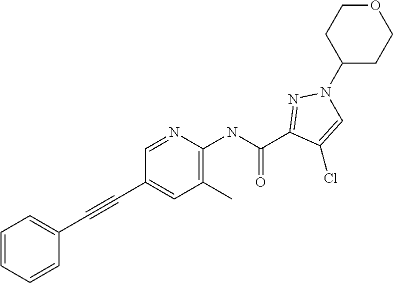 [SMILES: Cc1cc(C#Cc2ccccc2)cnc1NC(=O)c1nn(C2CCOCC2)cc1Cl] | 421 | −116 |
| 444 | 4-chloro-N-(3-methyl-5- (phenylethynyl)pyridin-2-yl)-1- ((tetrahydro-2H-pyran-4-yl)methyl)- 1H-pyrazole-3-carboxamide | 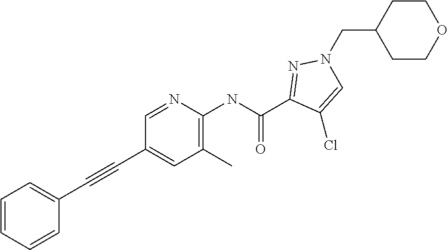 [SMILES: Cc1cc(C#Cc2ccccc2)cnc1NC(=O)c1nn(CC2CCOCC2)cc1Cl] | 435 | −116 |
| 445 | 1-(2-(1-acetylpiperidin-4-yl)ethyl)- 4-chloro-N-(5-((2- fluorophenyl)ethynyl)-3- methylpyridin-2-yl)-1H-pyrazole-3- carboxamide | 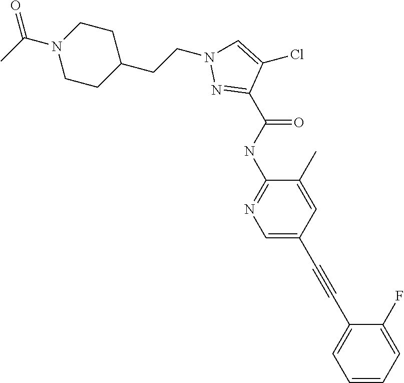 [SMILES: CC(=O)N1CCC(CCn2cc(Cl)c(C(=O)Nc3ncc(C#Cc4ccccc4F)cc3C)n2)CC1] | 508.4 | −115 |
| 446 | 4-chloro-N-(5-((4- fluorophenyl)ethynyl)-3- methylpyridin-2-yl)-1-(2-(1- isobutyrylpiperidin-4-yl)ethyl)-1H- pyrazole-3-carboxamide | 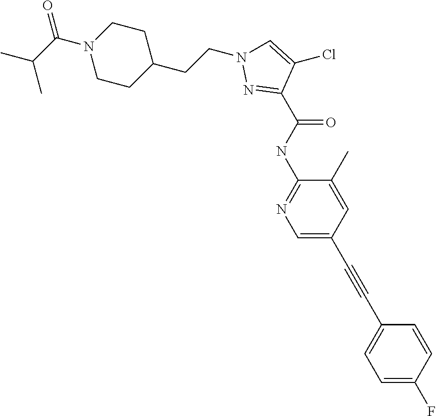 [SMILES: Cc1cc(C#Cc2ccc(F)cc2)cnc1NC(=O)c1nn(CCC2CCN(C(=O)C(C)C)CC2)cc1Cl] | 536.4 | −108 |
| 447 | 1-(2-(1-acetylpiperidin-4-yl)ethyl)- 4-chloro-N-(3-fluoro-5-(thiophen-2- ylethynyl)pyridin-2-yl)-1H- pyrazole-3-carboxamide | 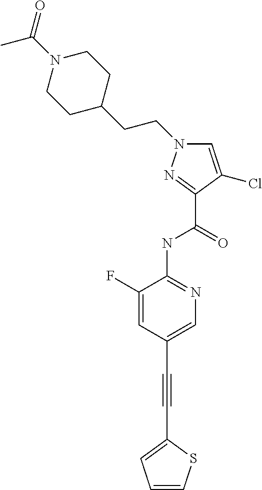 [SMILES: CC(=O)N1CCC(CCn2cc(Cl)c(C(=O)Nc3ncc(C#Cc4cccs4)cc3F)n2)CC1] | 500.3 | −107 |
| 448 | 1-(2-(1-(1,4-dioxane-2- carbonyl)piperidin-4-yl)ethyl)-4- chloro-N-(3-fluoro-5-(thiophen-2- ylethynyl)pyridin-2-yl)-1H- pyrazole-3-carboxamide | 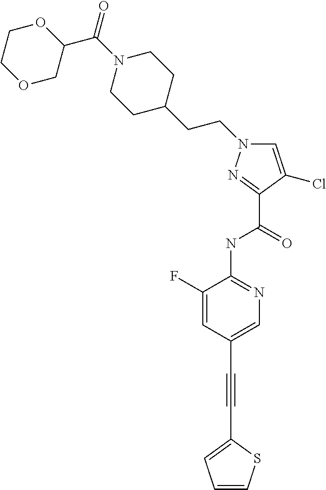 [SMILES: O=C(Nc1ncc(C#Cc2cccs2)cc1F)c1nn(CCC2CCN(C(=O)C3COCCO3)CC2)cc1Cl] | 572.1 | −106 |
| 449 | 1-((1,4-dioxan-2-yl)methyl)-4- chloro-N-(3-methyl-5- (phenylethynyl)pyridin-2-yl)-1H- pyrazole-3-carboxamide | 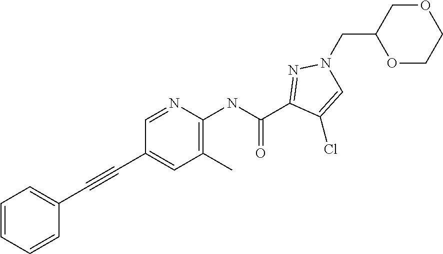 [SMILES: Cc1cc(C#Cc2ccccc2)cnc1NC(=O)c1nn(CC2COCCO2)cc1Cl] | 437 | −99 |
| 450 | 4-chloro-1-(2-(1- (cyclopropanecarbonyl)piperidin-4- yl)ethyl)-N-(5-((3- fluorophenyl)ethynyl)-3- methylpyridin-2-yl)-1H-pyrazole-3- carboxamide | 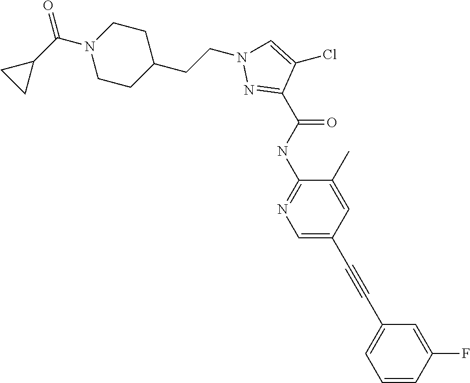 [SMILES: Cc1cc(C#Cc2cccc(F)c2)cnc1NC(=O)c1nn(CCC2CCN(C(=O)C3CC3)CC2)cc1Cl] | 534.4 | −94 |
| 451 | 4-chloro-1-(2-(1- (cyclopropanecarbonyl)piperidin-4- yl)ethyl)-N-(5-((4- fluorophenyl)ethynyl)-3- methylpyridin-2-yl)-1H-pyrazole-3- carboxamide | 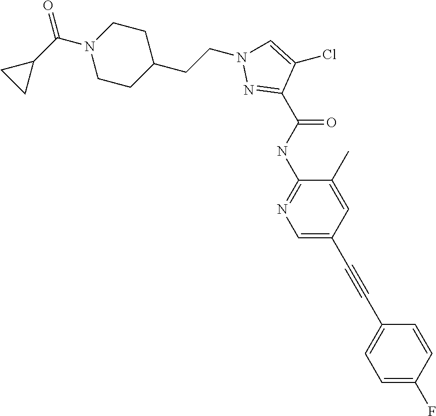 [SMILES: Cc1cc(C#Cc2ccc(F)cc2)cnc1NC(=O)c1nn(CCC2CCN(C(=O)C3CC3)CC2)cc1Cl] | 534.4 | −79 |
| 452 | 4-chloro-1-(2-(1- (cyclopropanecarbonyl)piperidin-4- yl)ethyl)-N-(3-fluoro-5-(thiophen-2- ylethynyl)pyridin-2-yl)-1H- pyrazole-3-carboxamide | 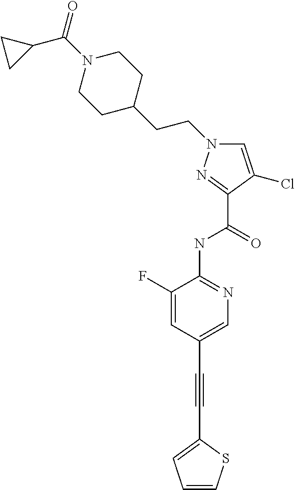 [SMILES: O=C(Nc1ncc(C#Cc2cccs2)cc1F)c1nn(CCC2CCN(C(=O)C3CC3)CC2)cc1Cl] | 526.2 | −62 |
| 453 | 4-chloro-N-(3-fluoro-5- (phenylethynyl)pyridin-2-yl)-1-(1- methyl-4-oxo-4,5,6,7-tetrahydro- 1H-indazol-6-yl)-1H-pyrazole-3- carboxamide | 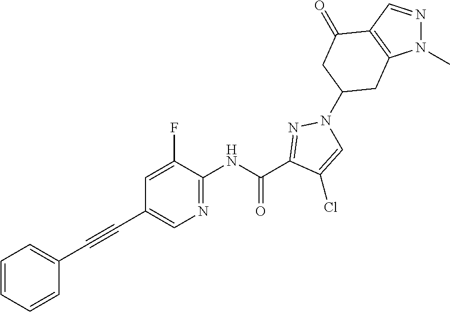 [SMILES: Cn1ncc2c1CC(n1cc(Cl)c(C(=O)Nc3ncc(C#Cc4ccccc4)cc3F)n1)CC2=O] | 458.3 | −56 |

### Example 1. In Vivo Pharmacology Test Results

MK-801 induced disruptions of Novel Object Recognition

Drugs: Test compounds were formulated in 10% Tween 80/90% sterile water vehicle. Following vigorous vortexing and sonicating with a hand-held homogenizer, the formulated compound was placed in an ultrasonic water bath for 1 hour at 39° C. Test compounds were formulated at a concentration that allowed for an oral (p.o.) administration of 10 mL dosing solution/kg body weight or intraperitoneal (i.p.) administration of a volume of 1 mL/kg body weight. MK-801 hydrogen maleate was obtained from Sigma (St. Louis, MO), dissolved in sterile saline, and dosed subcutaneously in a volume of 1 mL/kg body weight.

Animals: Adult male Sprague-Dawley rats (Envigo, Indianapolis, IN) weighing 175-300 g were used. They were housed in an animal care facility certified by the American Association for the Accreditation of Laboratory Animal Care (AALAC) under a 12-hour light/dark cycle (lights on: 6 a.m.; lights off: 6 p.m.) and had free access to food and water. Animals were acclimated to the housing facility for a minimum of five days before being tested and the behavioral testing was performed during the light phase. All experiments were approved by the Institutional Animals Care and Use Committee of Vanderbilt University and conformed to the guidelines established by the National Research Council Guide for the Care and Use of Laboratory Animals.

Apparatus: For the novel object recognition test an opaque Plastic chamber (41.2 cm×65.4 cm×34.9 cm) was used. At opposite ends of the chamber test objects (20-cm tall Gatorade bottles, filled with sterile water) or 22-cm tall metal boxes) could be placed). A video camera was mounted above the apparatus for recording the animal's behavior.

Procedure:

Habituation. At least one day prior to behavioral testing, animals were habituated to the empty testing chamber, i.e. in the absence of any objects, for ten minutes.

Training. Thirty minutes after administration of vehicle or MK-801, animals were placed into the testing chamber that contained two identical objects, either two traffic cones or two Magneto rods for a session duration of 10 minutes. Afterwards, animals were returned to their home cage.

Recognition. Ninety minutes after the end of the training session, animals were reintroduced to the test chamber where one of the two identical objects had been replaced by a novel object. (Example: training session with two traffic cones—recognition session with one traffic cone and one Magneto rod) for a total of five minutes.

Behavioral Analysis:

An observer blinded to treatment condition and novel object location used the video recordings to score the interaction of the animal with the two objects offline. The duration an animal explored each object was determined as the total time an animal was facing the object with its nose being≤2 cm away from the object and some discernible whisker movement being present. From these data, a Discrimination Index was calculated as follows:

Discrimination Index=100×(time exploring Novel object−time exploring Familiar object)/time exploring Novel object+time exploring Familiar object).

Data Analysis: Data were analyzed by one-way ANOVA followed by Dunnett's test if indicated using the GraphPad Prism 7 software package. A p-value; 0.05 was considered to represent a significant difference.

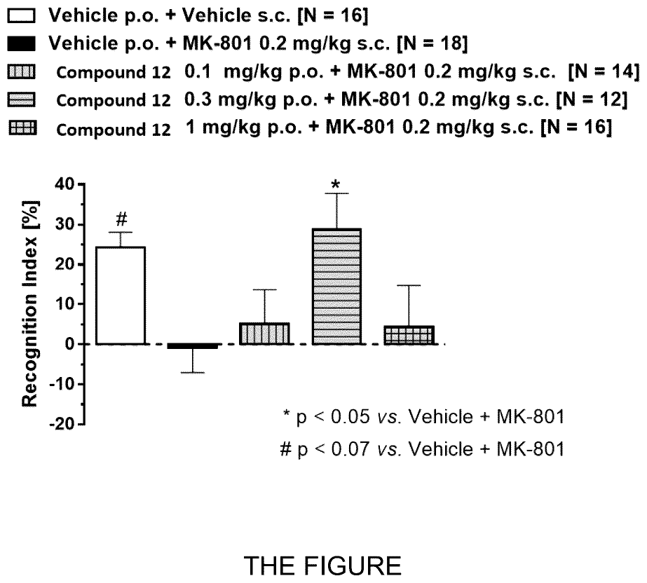

Results: Compound 12 dosed at 0.1 mg/kg, 0.3 mg/kg, and 1 mg/kg, and MK-801 dosed at 0.2 mg/kg, dose-dependently reversed of MK-801-induced deficits in the Novel Object Recognition Task, a preclinical rodent model of memory functions. Data are illustrated in FIG. 1.

Conclusions: Systemic administration of compound 12 caused reversal of MK801-induced deficits in the Novel Object Recognition Task, a preclinical rodent model of memory functions.

### Example 2. In Vitro Drug Metabolism and Pharmacokinetics

Compound 12 was tested in several in vitro assays to investigate plasma protein and brain homogenate binding, hepatic microsomal instrinsic clearance, and P450 inhibition. These assays were performed according to known methods as generally described in the following references: Conde-Ceide et al. ACS Med. Chem. Lett. 2015, 6, 716-720; Morris et al. J. Med. Chem. 2014, 57, 10192-10197; and Bubser et al. ACS Chem. Neurosci. 2014, 5, 920-942. Results are presented in Table 4. Abbreviations which have been used in the descriptions of the following tables are; PPB is plasma protein binding; BHB is brain homogenate binding; and CLᵢₙₜ is intrinsic clearance in hepatic microsomes.

TABLE 4
In vitro DPMK for Compound 12

| Assay | Result |
|---|---|
| Rat PPB (% bound) | 98.4 |
| Mouse PPB (% bound) | 99.2 |
| Human PPB (% bound) | 98.8 |
| Rat BHB (% bound) | 99.7 |
| Mouse BHB (% bound) | 99.7 |
| Rat CLᵢₙₜ (mL/min/kg) | 54 |
| Mouse CLᵢₙₜ (mL/min/kg) | 103 |
| Human CLᵢₙₜ (mL/min/kg) | 16 |
| P450 3A4 IC₅₀ (μM) | >30 |
| P450 2D6 IC₅₀ (μM) | 4.5 |
| P450 2C9 IC₅₀ (μM) | 5.7 |
| P450 1A2 IC₅₀ (μM) | 8.6 |

### Example 3. In Vivo Drug Metabolism and Pharmacokinetics

Compound 12 was tested in several in vivo assays to investigate pharmacokinetics and brain distribution. Results from rat i.v. cassette injection are presented in Table 5. Abbreviations which have been used in the descriptions of following tables are; CLₚ is plasma clearance; Vₛₛ is volume of distribution at steady state; Elim. t_{1/2} is half-life for elimination; Kₚ is tissue distribution partition coefficient; K_{p,uu} is unbound tissue distribution partition coefficient.

TABLE 5
In vivo DMPK for Compound 12

| Assay | Result |
|---|---|
| Rat CLₚ (mL/min/kg) | 28 |
| Rat Vₛₛ (L/kg) | 4.7 |
| Rat Elim. t_{1/2} (hr) | 2.0 |
| Rat brain: plasma Kₚ | 0.97 |
| Rat brain: plasma K_{p, uu} | 0.19 |

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

The present compound has strong TREK inhibition, and thus is useful as a therapeutic and/or prophylactic agent for various the neurological and/or psychiatric disorders associated with TREK-1, TREK-2 or both TREK-1 and TREK-2 channel activity, particularly, depression, schizophrenia, anxiety disorders, bipolar disorder, Alzheimer's disease, Parkinson's disease, Huntington's disease, Amyotrophic lateral sclerosis, 22q11.2 deletion syndrome, neuropathic pain, cerebral infarction.

The present compound has strong TREK activation, and thus is useful as a prophylactic and/or therapeutic agent for various disorders associated with TREK-1, TREK-2 or both TREK-1 and TREK-2 channel dysfunction, particularly, pain, migraine, nasal inflammation, atrial fibrillation, acute respiratory distress syndrome, acute lung injury, overactive bladder, cerebral ischemia, epilepsy, amyotrophic lateral sclerosis, neuronal degenerative diseases (e.g. Alzheimer's disease), sepsis, pancreatic cancer, Cushing's syndrome, autosomal dominant polycystic kidney disease, bone fracture, osteoporosis, temporal lobe epilepsy, schizophrenia, colitis, or addiction.

1. A compound of formula (I): 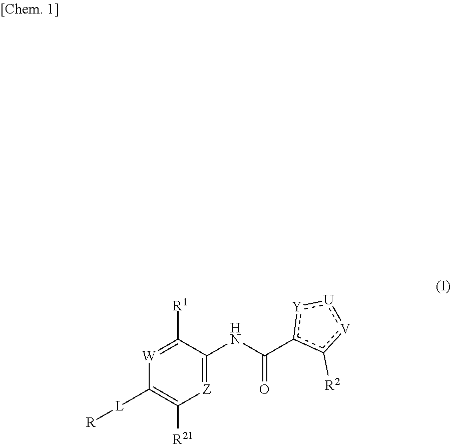
or a pharmaceutically acceptable salt thereof,
wherein:
L is selected from (1) bond, (2) C2-C4-alkynylene, (3) C2-C4-alkenylene, (4) —(C1-C10-alkylene)-O—, (5) —O—(C1-C10-alkylene)-, (6) -(6 to 15 membered aryl)-, (7) -(5 to 15 membered heteroaryl)-, (8) -(3 to 15 membered heterocycle)-, and (9) —(C3-C10-cycloalkane)-;
W is selected from (1) CH, (2) CR⁴, and (3) N
Z is selected from (1) CH, (2) CR⁵, and (3) N
R⁴ and R⁵ is each independently selected from (1) cyano, (2) halogen, (3) pentahalosulfanyl, (4) C1-C10-thioalkyl, (5) C1-C10-alkoxy, (6) C1-C10-alkyl, (7) C2-C10-alkenyl, (8) C2-C10-alkynyl, (9) —OR^{b}, (10) 6 to 15 membered aryl, (11) 5 to 15 membered heteroaryl, (12) C3-C10-cycloalkyl, (13) C2-C10-heteroalkyl, (14) 3 to 15 membered heterocycle, and (15) —(CR^{c}R^{d})ₙ-Q,
wherein each of (4)-(8) in R⁴ or R⁵ may be optionally substituted with 1 to 10 halogen, and each of (10)-(14) in R⁴ or R⁵ may be optionally substituted with 1 to 10 substituents selected from (1) halogen, (2) C1-C10-alkyl, and (3) C1-C10-haloalkyl;
R^{b} is selected from (1) hydrogen, (2) C1-C10-alkyl, and (3) C1-C10-haloalkyl;
R^{c} is selected from (1) hydrogen, (2) halogen, (3) C1-C10-alkyl, and (4) C1-C10-haloalkyl;
R^{d} is selected from (1) hydrogen, (2) halogen, (3) C1-C10-alkyl and (4) C1-C10-haloalkyl;
or when R^{c} and R^{d} are C1-C10-alkyl, R^{c} and R^{d} may optionally form a C3-C10-cycloalkyl together with the carbon atom to which they are attached;
n is 0, 1, 2, 3 or 4;
Q is selected from (1) halogen, (2) cyano, (3) —OR²⁰¹, (4) —SR²⁰², (5) —C(═O)R²⁰³, (6) —C(═O)OR²⁰⁴, (7) —S(═O)R²⁰⁵, (8) —SO₂R²⁰⁶, (9) —N(R²⁰⁷)₂, (10) —C(═O)N(R^{20′})₂, (11) —SO₂N(R²⁰⁹)₂, (12) 6 to 15 membered aryl, (13) 5 to 15 membered heteroaryl, (14) C3-C10-cycloalkyl, and (15) 3 to 15 membered heterocycle, wherein each of (12)-(15) in Q may be optionally substituted with 1 to 10 substituents selected from (1) halogen, (2) C1-C10-alkyl, and (3) C1-C10-haloalkyl;
R²⁰¹, R²⁰², R²⁰³, R²⁰⁴, R²⁰⁵, R²⁰⁶, R²⁰⁷, R²⁰⁸, and R²⁰⁹ are each independently selected from (1) hydrogen, (2) C1-C10-alkyl, and (3) C1-C10-haloalkyl, and multiple R²⁰⁷, R²⁰⁸, and R²⁰⁹ may be the same as or different from each other; 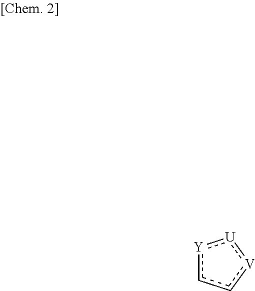
is selected from (1) 1,2,3-triazole substituted with R³, (2) pyrrole substituted with R³, (3) pyrazole substituted with R³, (4) isoxazole substituted with R³, (5) isothiazole substituted with R³, (6) imidazole substituted with R³, (7) furan substituted with R³, (8) thiophene substituted with R³;
wherein Y is selected from (1) CH, (2) CR³, (3) N, (4) NH, (5) NR³, (6) O and (7) S,
U is selected from (1) CH, (2) CR³, (3) CR^{x}, (4) N, (5) NH, (6) NR³, (7) NR^{x}, (8) O and (9) S,
V is selected from (1) CH, (2) CR^{x}, (3) N, (4) NH, (5) NR^{x}, (6) O, and (7) S,
one of Y and U is NR³ or CR³, and the other is not NR3 or CR³,
[Chem. 3] [symbol]
is single bond or double bond,
R^{x} is selected from (1) NH₂, (2) halogen, (3) C1-C4-alkyl, and (4) C1-C4-haloalkyl;
R is selected from (1) 6 to 15 membered aryl and (2) 5 to 15 membered heteroaryl, each of which may be optionally substituted with 1 to 5 R⁶;
wherein multiple R⁶ may be the same as or different from each other;
R⁶ is selected from (1) halogen, (2) cyano, (3) pentahalosulfanyl, (4) C1-C10-alkyl, (5) C1-C10-thioalkyl, (6) C1-C10-alkoxy, (7) C2-C10-alkenyl, (8) C2-C10-alkynyl, and (9) —OR^{b}, wherein each of (4)-(6) in R⁶ is may be optionally substituted with 1 to 10 halogen;
R¹ is selected from (1) C1-C10-alkyl, (2) halogen, (3) C1-C10-alkoxy, (4) C1-C10-haloalkyl, (5) C1-C10-haloalkoxy and (6) cyano;
R²¹ is selected from (1) hydrogen, (2) C1-C10-alkyl, (3) halogen, (4) C1-C10-alkoxy, (5) C1-C10-haloalkyl, (6) C1-C10-haloalkoxy, and (7) cyano;
R² is selected from (1) halogen, (2) C1-C10-alkyl, (3) C1-C10-haloalkyl, (4) cyano, and (5) C3-C10-cycloalkyl which may be optionally substituted with 1 to 10 substituents selected from (1) halogen (2) C1-C10-alkyl, and (3) C1-C10-haloalkyl;
R³ is selected from (1) R⁷ or 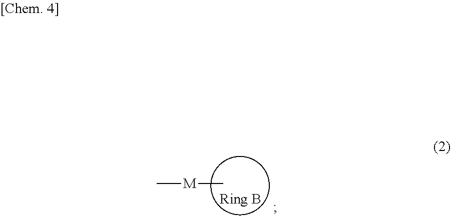
wherein R⁷ is selected from (1) C3-C10-alkyl, (2) —(C2-C10-alkylene)-cyano, (3) —(C1-C10-alkylene)-NH—C(═O)—O—(C1-C10-alkyl), (4) —(C1-C10-alkylene)-O—(C1-C10-alkyl), and (5) —(C1-C10-alkylene)-(CR⁷¹R⁷²)ₚ—R⁷³—, each of (1)-(5) in R⁷ is may be optionally substituted with 1 to 10 halogen;
wherein R⁷¹ is selected from (1) hydrogen, (2) halogen, (3) C1-C10-alkyl and (4) C1-C10-haloalkyl;
R⁷² is selected from (1) hydrogen, (2) halogen, (3) C1-C10-alkyl, and (4) C1-C10-haloalkyl;
or when R⁷¹ and R⁷² are C1-C10-alkyl, R⁷¹ and R⁷² may optionally form a C3-C10-cycloalkyl together with the carbon atom to which they are attached, wherein C3-C10-cycloalkyl may be optionally substituted with 1-10 substituents selected from (1) halogen and (2) cyano;
p is 0, 1, 2, 3 or 4;
R⁷³ is selected from (1) —OR¹⁰¹, (2) —SR¹⁰², (3) —C(═O)R¹⁰³, (4) —C(═O)OR¹⁰⁴, (5) S(═O)R¹⁰⁵, (6) —SO₂R¹⁰⁶, (7) —N(R¹⁰⁷)₂, (8) —C(═O)N(R¹⁰⁸)₂, (9) —SO₂N(R¹⁰⁹)₂, (10) 6 to 15 membered aryl, (11) 5 to 15 membered heteroaryl, (12) C3-C10-cycloalkyl, and (13) 3 to 15 membered heterocycle, wherein each of (10)-(13) in R⁷³ may be optionally substituted with 1-10 substituents selected from (1) halogen, (2) C1-C10-alkyl, and (3) C1-C10-haloalkyl;
R¹⁰¹, R¹⁰², R¹⁰³, R¹⁰⁴, R¹⁰⁵, R¹⁰⁶, R¹⁰⁷, R¹⁰⁸, and R¹⁰⁹ are each independently selected from (1) hydrogen, (2) C1-C10-alkyl, and (3) C1-C10-haloalkyl, and multiple R¹⁰⁷, R¹⁰⁸, and R¹⁰⁹ may be the same as or different from each other;
M is selected from (1) bond or (2) C1-C10-alkylene which may be optionally substituted with 1 to 3 halogens;
Ring B is selected from (1) C3-C10-cycloalkyl which may be optionally substituted with 1 to 5 R⁸, (2) 3 to 15 membered heterocycle which may be optionally substituted with 1 to 5 R⁹, (3) 6 to 15 membered aryl which may be optionally substituted with 1 to 5 R¹⁰, and (4) 5 to 15 membered heteroaryl which may be optionally substituted with 1 to 5 R¹¹;
wherein multiple R⁸, R⁹, R¹⁰ or R¹¹ may be the same as or different from each other;
R⁸, R⁹, R¹⁰, and R¹¹ are each independently selected from (1) halogen, (2) C1-C10-alkyl, (3) cyano, (4) —C(═O)—(C1-C10-alkyl), (5) —C(═O)—(C3-C10-cycloalkyl), (6) —C(═O)—(3 to 15 membered heterocycle), (7) —C(═O)—O—(C1-C10-alkyl), (8) —C(═O)—O—(C3-C10-cycloalkyl), (9) —C(═O)—O-(3 to 15 membered heterocycle), (10) —C(═O)—NR⁸¹—(C1-C10-alkyl), (11) —C(═O)NR⁸²—(C3-C10-cycloalkyl), (12) —C(═O)—NR⁸³-(3 to 15 membered heterocycle), (13) —NR⁸⁴—C(═O)—(C1-C10-alkyl), (14) —NR⁸⁵—C(═O)—(C3-C10-cycloalkyl), (15) —NR⁸⁶—C(═O)—(3 to 15 membered heterocycle), (16) —NR⁸⁷—C(═O)—O—(C1-C10-alkyl), (17) —NR⁸⁸—C(═O)—O—(C3-C10-cycloalkyl), (18) —NR⁸⁹—C(═O)—O-(3 to 15 membered heterocycle), (19) —SO₂—(C1-C10-alkyl), and (20) oxo;
wherein each of (2), (4), (7), (10), (13), (16) and (19) in R⁸, R⁹ R¹⁰ or R¹¹ may be optionally substituted with 1 to 10 substituents selected from (1) halogen, (2) —OH, and (3) C1-C10-alkoxy, and each of (5), (6), (8), (9), (11), (12), (14), (15), (17), and (18) in R⁸, R⁹ R¹⁰ or R¹¹ may be optionally substituted with 1 to 10 substituents selected from (1) halogen, (2) OH, (3) C1-C10-alkyl, (4) C1-C10-alkoxy, and (5) C1-C10-haloalkyl; and
R⁸¹, R⁸², R⁸³, R⁸⁴, R⁸⁵, R⁸⁶, R⁸⁷, R⁸⁸, and R⁸⁹ are independently selected from (1) hydrogen and (2) C1-C4-alkyl.

2. The compound or a pharmaceutically acceptable salt thereof according to claim 1, which is a compound of formula (Ia): 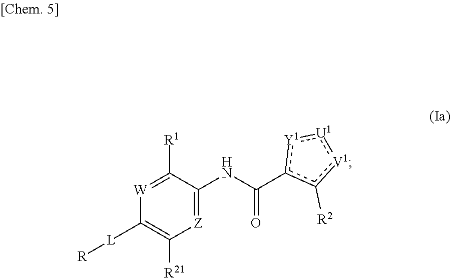
wherein 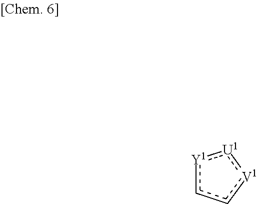
is selected from (1) 1,2,3-triazole substituted with R³, (2) pyrrole substituted with R³, (3)pyrazole substituted with R³, and (4) imidazole substituted with R³; wherein Y¹ is independently selected from CR³ or NR³;
U¹ is selected from (1) CH, (2) NR^{x}, and (3) N; and
V¹ is selected from (1) CH and (2) N.

3. The compound or a pharmaceutically acceptable salt thereof according to claim 2, which is a compound of formula (Ia-1):

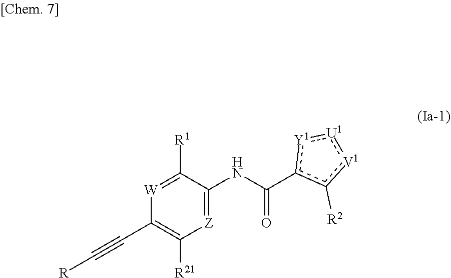

4. The compound or a pharmaceutically acceptable salt thereof according to claim 3, wherein R² is halogen.

5. The compound or a pharmaceutically acceptable salt thereof according to claim 4, which is a compound of formula (Ia-2): 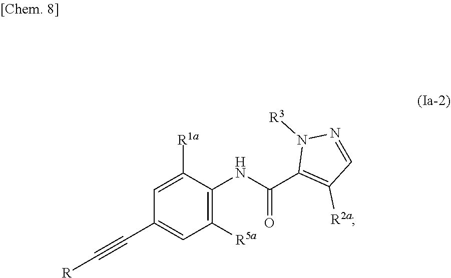
wherein
R^{1a} is selected from (1) halogen and (2) C1-C10-alkyl;
R^{5a} is selected from (1) hydrogen, (2) halogen and (3) C1-C10-alkyl; and
R^{2a} is halogen.

6. The compound or a pharmaceutically acceptable salt thereof according to claim 5, which is a compound of formula (Ia-3):

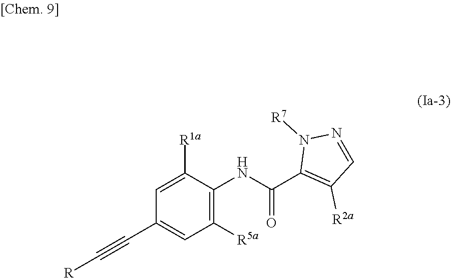

7. The compound or a pharmaceutically acceptable salt thereof according to claim 5, which is a compound of formula (Ia-4):

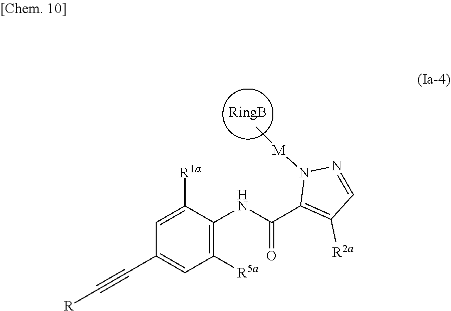

8. The compound or a pharmaceutically acceptable salt thereof according to claim 4, which is a compound of formula (Ia-5): 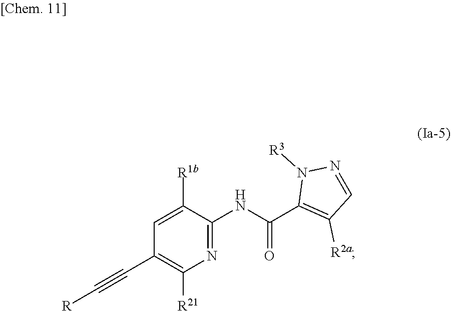
wherein R^{1b} is selected from (1) halogen and (2) C1-C10-alkyl.

9. The compound or a pharmaceutically acceptable salt thereof according to claim 8, which is a compound of formula (Ia-6):

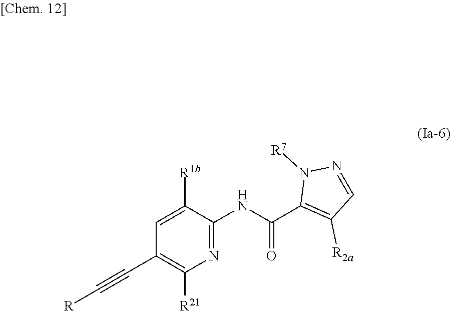

10. The compound or a pharmaceutically acceptable salt thereof according to claim 8, which is a compound of formula (Ia-7):

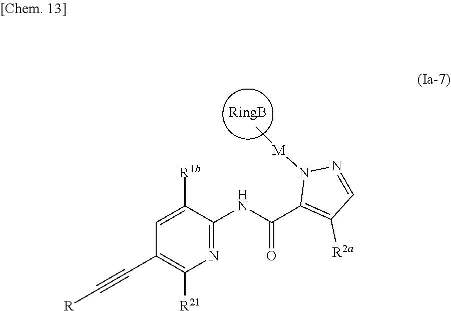

11. The compound or a pharmaceutically acceptable salt thereof according to claim 1, which is a compound of formula (Ib): 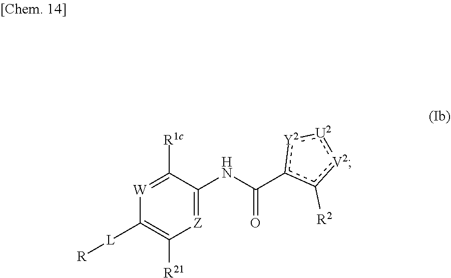
wherein 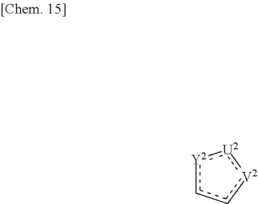
is selected from (1) 1,2,3-triazole substituted with R³, (2) pyrrole substituted with R³, (3) pyrazole substituted with R³, and (4) imidazole substituted with R³;
wherein Y² is selected from (i) CH and (2) N;
U² is selected from (1) CR³ and (2) NR³;
V² is selected from (1) CH, (2) CR, and (3) N; and
R^{1c} is selected from (1) C1-C10-alkyl, (2) halogen, and (3) C1-C10-haloalkyl.

12. The compound or a pharmaceutically acceptable salt thereof according to claim 11, which is a compound of formula (Ib-1):

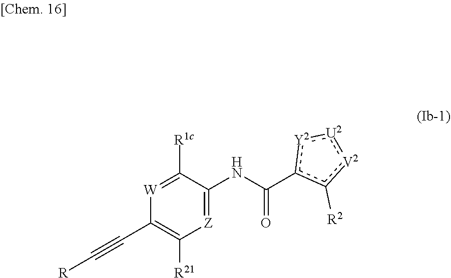

13. The compound or a pharmaceutically acceptable salt thereof according to claim 12, which is a compound of formula (Ib-2):

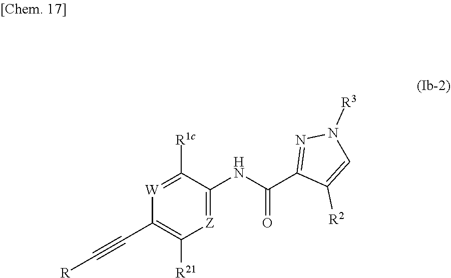

14. The compound or a pharmaceutically acceptable salt thereof according to claim 13, which R² is halogen.

15. The compound or a pharmaceutically acceptable salt thereof according to claim 14, which is a compound of formula (Ib-3): 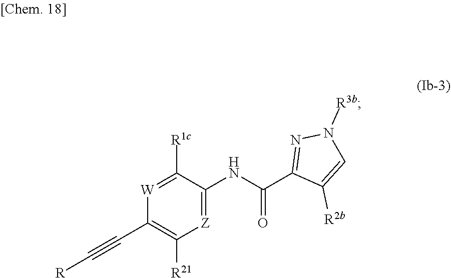
wherein R^{2b} is halogen;
R^{3b} is 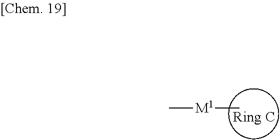
wherein M¹ is selected from (1) bond and (2) C1-C10-alkylene which may be optionally substituted with 1 to 3 halogen,
Ring C is selected from (1) C3-C10-cycloalkyl which may be optionally substituted with 1 to 5 R¹³, (2) 3 to 15 membered heterocycle which may be optionally substituted with 1 to 5 R¹⁴, and (3) 5 to 15 membered heteroaryl which may be optionally substituted with 1 to 5 R¹⁵;
wherein multiple R¹³, R¹⁴, or R¹⁵ may be the same as or different from each other; and
R¹³, R¹⁴, and R¹⁵ are each independently selected from (1) halogen, (2) C1-C10-alkyl, (3) —C(═O)—(C1-C10-haloalkyl), (4) cyano, (5) —C(═O)—(C1-C10-alkyl), (6) —C(═O)—(C3-C10-cycloalkyl), (7) —C(═O)—(3 to 15 membered heterocycle), and (8) oxo.

16. The compound or a pharmaceutically acceptable salt thereof according to claim 1, wherein the compound is
(1) 1-((1,4-dioxan-2-yl)methyl)-4-chloro-N-(3-methyl-5-(phenylethynyl)pyridin-2-yl)-1H-pyrazole-5-carboxamide,
(2) 4-chloro-N-(3-methyl-5-(phenylethynyl)pyridin-2-yl)-1-((tetrahydro-2H-pyran-4-yl)methyl)-1H-pyrazole-5-carboxamide,
(3) (R)-4-chloro-N-(3-methyl-5-(phenylethynyl)pyridin-2-yl)-1-(tetrahydrofuran-3-yl)-1H-pyrazole-5-carboxamide,
(4) (S)-1-((1,4-dioxan-2-yl)methyl)-4-chloro-N-(3-fluoro-5-(phenylethynyl)pyridin-2-yl)-1H-pyrazole-5-carboxamide,
(5) 1-(1-acetylpiperidin-4-yl)-4-chloro-N-(3-methyl-5-(phenylethynyl)pyridin-2-yl)-1H-pyrazole-5-carboxamide,
(6) 1-(1-acetylpiperidin-4-yl)-4-chloro-N-(3-fluoro-5-(phenylethynyl)pyridin-2-yl)-1H-pyrazole-5-carboxamide,
(7) 1-(1-acetylpiperidin-4-yl)-4-chloro-N-(3-fluoro-5-((4-fluorophenyl)ethynyl)pyridin-2-yl)-1H-pyrazole-5-carboxamide,
(8) 1-(1-acetylpiperidin-4-yl)-4-chloro-N-(5-((4-fluorophenyl)ethynyl)-3-methylpyridin-2-yl)-1H-pyrazole-5-carboxamide,
(9) 4-chloro-N-(3-methyl-5-(phenylethynyl)pyridin-2-yl)-1-(4,5,6,7-tetrahydropyrazolo[1,5-a]pyridin-5-yl)-1H-pyrazole-5-carboxamide,
(10) 1-((3R,4R)-1-acetyl-3-fluoropiperidin-4-yl)-4-chloro-N-(5-((4-fluorophenyl)ethynyl)-3-methylpyridin-2-yl)-1H-pyrazole-5-carboxamide,
(11) 1-(1-(1,4-dioxane-2-carbonyl)piperidin-4-yl)-4-chloro-N-(3-fluoro-5-(phenylethynyl)pyridin-2-yl)-1H-pyrazole-5-carboxamide,
(12) 1-(cis-4-acetamidocyclohexyl)-4-chloro-N-(3-methyl-5-(phenylethynyl)pyridin-2-yl)-1H-pyrazole-5-carboxamide,
(13) 1-(cis-4-acetamidocyclohexyl)-4-chloro-N-(3-fluoro-5-(phenylethynyl)pyridin-2-yl)-1H-pyrazole-5-carboxamide,
(14) 1-(cis-4-acetamidocyclohexyl)-4-chloro-N-(5-((4-fluorophenyl)ethynyl)-3-methylpyridin-2-yl)-1H-pyrazole-5-carboxamide,
(15) 1-((1-acetyl-4-fluoropiperidin-4-yl)methyl)-4-chloro-N-(3-methyl-5-(phenylethynyl)pyridin-2-yl)-1H-pyrazole-5-carboxamide,
(16) (S)-1-(1-(1-acetylpiperidin-4-yl)ethyl)-4-chloro-N-(3-methyl-5-(phenylethynyl)pyridin-2-yl)-1H-pyrazole-5-carboxamide,
(17) 1-((1-acetyl-3-fluoroazetidin-3-yl)methyl)-4-chloro-N-(3-fluoro-5-(phenylethynyl)pyridin-2-yl)-1H-pyrazole-5-carboxamide,
(18) 1-((1-acetyl-3-fluoroazetidin-3-yl)methyl)-4-chloro-N-(3-methyl-5-(phenylethynyl)pyridin-2-yl)-1H-pyrazole-5-carboxamide,
(19) 4-chloro-1-[(3S)-1-(cyclopropanecarbonyl)pyrrolidin-3-yl]-N-[3-methyl-5-(phenylethynyl)pyridin-2-yl]-1H-pyrazole-5-carboxamide, or
(20) 4-chloro-1-[(3R)-1-(cyclopropanecarbonyl)pyrrolidin-3-yl]-N-[3-methyl-5-(phenylethynyl)pyridin-2-yl]-1H-pyrazole-5-carboxamide.

17. A pharmaceutical composition comprising the compound or a pharmaceutically acceptable salt thereof according to claim 1, and a pharmaceutically acceptable carrier.

18. A pharmaceutical comprising the compound or a pharmaceutically acceptable salt thereof according to claim 2, and a pharmaceutically acceptable carrier.

19. The pharmaceutical composition according to claim 18, wherein the compound or pharmaceutically acceptable salt thereof is a TREK1, TREK2, or both TREK1 and TREK2 inhibitor.

20. A pharmaceutical composition comprising the compound or a pharmaceutically acceptable salt thereof according to claim 11, and a pharmaceutically acceptable carrier.

21. The pharmaceutical composition according to claim 20, wherein the compound or pharmaceutically acceptable salt thereof is a TREK1, TREK2, or both TREK1 and TREK2 activator.

22. A method for treating a disorder associated with TREK1, TREK2 or dual TREK1/TREK2 dysfunction in which inhibitors of TREK1, TREK2, or both TREK1 and TREK2 would offer therapeutic benefit in a mammal, comprising administering to the mammal in need thereof a therapeutically effective amount a compound or pharmaceutically acceptable salt thereof according to claim 2.

23. The method according to claim 22, wherein a disorder associated with TREK1, TREK2 or dual TREK1/TREK2 dysfunction in which inhibitors of TREK1, TREK2, or both TREK1 and TREK2 would offer therapeutic benefit in a mammal is a neurological and/or psychiatric disorder.

24. The method according to claim 23, wherein the neurological and/or psychiatric disorder is selected from depression, schizophrenia, anxiety disorders, bipolar disorder, Alzheimer's disease, Parkinson's disease, Huntington's disease, Amyotrophic lateral sclerosis, 22q11.2 deletion syndrome, neuropathic pain or cerebral infarction.

25. A method for treating a disorder associated with TREK1, TREK2 or dual TREK1/TREK2 dysfunction in which activators of TREK1, TREK2, or both TREK1 and TREK2 would offer therapeutic benefit, comprising administering to a mammal in need thereof a therapeutically effective amount a compound or pharmaceutically acceptable salt thereof according to claim 11.

26. The method according to claim 25, wherein the disorder associated with TREK1, TREK2, or dual TREK1/TREK2 dysfunction in which activators of TREK1, TREK2, or both TREK1 and TREK2 would offer therapeutic benefit is selected from pain, migraine, nasal inflammation, atrial fibrillation, acute respiratory distress syndrome, acute lung injury, overactive bladder, cerebral ischemia, epilepsy, amyotrophic lateral sclerosis, neuronal degenerative diseases (e.g. Alzheimer's disease), sepsis, pancreatic cancer, Cushing's syndrome, autosomal dominant polycystic kidney disease, bone fracture, osteoporosis, temporal lobe epilepsy, schizophrenia, colitis, or addiction.

27. A kit comprising a compound or a pharmaceutically acceptable salt thereof according to claim 2, and one or more of: (a) at least one agent known to decrease TREK1 channel activity; (b) at least one agent known to decrease TREK2 channel activity; (c) at least one agent known to prevent and/or treat a disorder associated with TREK channel dysfunction in which inhibitors of TREK1, TREK2, or both TREK1/TREK2 would offer therapeutic benefit in a mammal; (d) instructions for preventing and/or treating a disorder associated with TREK channel dysfunction in which inhibitors of TREK1, TREK2, or both TREK1/TREK2 would offer therapeutic benefit in a mammal; and (e) instructions for administering the compound in connection with cognitive behavioral therapy.

28. A kit comprising a compound or a pharmaceutically acceptable salt thereof according to claim 11, and one or more of: (a) at least one agent known to increase TREK1 channel activity; (b) at least one agent known to increase TREK2 channel activity; (c) at least one agent known to prevent and/or treat a disorder associated with TREK channel activity in which activators of TREK1, TREK2, or both TREK1/TREK2 would offer therapeutic benefit in a mammal; and (d) instructions for preventing and/or treating a disorder associated with TREK activity in which activators of TREK1, TREK2, or both TREK1/TREK2 would offer therapeutic benefit in a mammal.
